(19) United States
(12) Patent Application Publication
(10) Pub. No.: US 2004/0229848 A1
(43) Pub. Date: Nov. 18, 2004

(54) Glutaminyl based DP IV-inhibitors

(76) Inventors: Hans-Ulrich Demuth, Halle/Saale (DE); Matthias Hoffmann, Wengelsdorf (DE); Torsten Hoffmann, Halle/Saale (DE); Andre J. Niestroj, Sennewitz (DE); Stephan Schilling, Halle/Saale (DE); Ulrich Heiser, Halle/Saale (DE)

Correspondence Address: Brown Rudnick Berlack Israels LLP, One Financial Center, Box IP, Boston, MA, 02111, US

(21) Appl. No.: 10/839,122

(22) Filed: May 5, 2004

Related U.S. Application Data
(60) Provisional application No. 60/467,914, filed on May 5, 2003.
(60) Provisional application No. 60/468,014, filed on May 5, 2003.

(51) Int. Cl. C07F 9/22; A61K 31/69; A61K 31/66

(52) U.S. Cl. 514/114; 514/563; 514/357; 514/408; 514/616; 546/334; 548/567; 562/450; 562/15; 514/64

(57) ABSTRACT

The present invention relates dipeptidyl peptidase IV inhibition and, more particularly, relates to glutaminyl derivatives, wherein the glutamin residue is bound in a peptide manner to a moiety which imitates the amino acid residue prolin, especially to a nitrogen containing moiety, pharmaceutical compositions containing said compounds, and the use of said compounds in inhibiting dipeptidyl peptidase IV and dipeptidyl peptidase IV-like enzyme activity.

### FIELD OF THE INVENTION

The present invention relates to the area of dipeptidyl peptidase IV inhibition and, more particularly, relates to glutaminyl derivatives, wherein the glutamin residue is bound in a peptide manner to a moiety which imitates the amino acid residue prolin, especially to a nitrogen containing moiety, pharmaceutical compositions containing said compounds, and the use of said compounds in inhibiting dipeptidyl peptidase IV and dipeptidyl peptidase IV—like enzyme activity.

Further, the present invention concerns the metabolism of the glutamin residue of these glutamin based DP IV inhibitors, which are metabolized and inactivated enzymatically to cyclic compounds by the enzyme glutaminyl cyclase (QC).

It is an aspect of the present invention to provide new DPIV inhibitors, optionally in combination with glutaminyl cyclase (QC) inhibitors, which are effective e.g. in treating conditions mediated by inhibition of DPIV and DPIV-iike enzymes, pharmaceutical compositions e.g. useful in inhibiting DPIV and DPIV-like enzymes and/or inhibiting QC and QC-like enzymes, and a method of inhibiting said enzyme activities.

Another aspect of the invention relates to a method of treatment, in particular to a method for the treatment of diabetes mellitus, especially non-insulin dependent diabetes (NIDDM) or Type 2 diabetes and conditions associated with diabetes mellitus and to compositions for use in such method.

### BACKGROUND ART

Dipeptidyl peptidase IV (DPIV) is a serine protease which cleaves N-terminal dipeptides from a peptide chain containing, preferably, a proline residue in the penultimate position. Although the biological role of DPIV in mammalian systems has not been completely established, it is believed to play an important role in neuropeptide metabolism, T-cell activation, attachment of cancer cells to the endothelium and the entry of HIV into lymphoid cells.

Likewise, it was discovered that DPIV is responsible for inactivating glucagon-like peptide-1 (GLP-1) and glucose-dependent insulinotropic peptide also known as gastric-inhibitory peptide (GIP). Since GLP-1 is a major stimulator of pancreatic insulin secretion and has direct beneficial effects on glucose disposal, in WO 97/40832 and U.S. Pat. No. 6,303,661 inhibition of DPIV and DPIV-like enzyme activity was shown to represent an attractive approach e.g. for treating non-insulin-dependent diabetes mellitus (NIDDM).

Dipeptidyl peptidase IV (DPIV) is a post-proline (to a lesser extent post-alanine, post-serine or post-glycine) cleaving serine protease found in various tissues of the body including kidney, liver, and intestine.

It is known that DPIV inhibitors may be useful for the treatment of impaired glucose tolerance and diabetes mellitus (International Patent Application, Publication Number WO 99/61431, Pederson R A et al, Diabetes. 1998 August; 47(8):1253-8 and Pauly RP et al, Metabolism 1999 March;

48(3):385-9). In particular WO 99/61431 discloses DPIV inhibitors comprising an amino acid residue and a thiazolidine or pyrrolidine group, and salts thereof, especially L-threo-isoleucyl thiazolidine, L-allo-isoleucyl thiazolidine, L-threo-isoleucyl pyrrolidine, L-allo-isoleucyl thiazolidine, L-allo-isoleucyl pyrrolidine, and salts thereof. In particular PCT/EP 02/07124 discloses DPIV inhibitors comprising an glutaminyl residue and a thiazolidine or pyrrolidine group, and salts thereof, especially glutaminyl thiazolidine and glutaminyl pyrrolidine, and salts thereof.

Further examples for low molecular weight dipeptidyl peptidase IV inhibitors are agents such as tetrahydroisoquinolin-3-carboxamide derivatives, N-substituted 2-cyanopyroles and -pyrrolidines, N—(N′-substituted glycylY2-cyanopyrrolidines, N-(substituted glycyl)-thiazolidines, N-(substituted glycyl)-4-cyanothiazolidines, boronyl inhibitors and cyclopropyl-fused pyrrolidines. Inhibitors of dipeptidyl peptidase IV are described in U.S. Pat. No. 6,011,155; U.S. Pat. No. 6,107,317; U.S. Pat. No. 6,110,949; U.S. Pat. No. 6,124,305; U.S. Pat. No. 6,172,081; WO 99/61431, WO 99/67278, WO 99/67279; DE 198 34 591, WO 97/40832, DE 196 16 486 C₂, WO 95/15309, WO 98/19998, WO 00/07617, WO 99/38501, WO 99/46272, WO 99/38501, WO 01/68603, WO 01/40180, WO 01/81337, WO 01/81304, WO 01/55105, WO 02/02560, WO 01/34594, WO 02/38541 (Japanese), WO 02/083128, WO 03/072556, WO 03/002593, WO 03/000250, WO 03/000180, WO 03/000181, EP 1 258 476, WO 03/002553, WO 03/002531, WO 03/002530, WO 03/004496, WO 03/004498, WO 03/024942, WO 03/024965, WO 03/033524, WO 03/035057, WO 03/035067, WO 03/037327, WO 03/040174, WO 03/045977, WO 03/055881, WO 03/057144, WO 03/057666, WO 03/068748, WO 03/068757, WO 03/082817, WO 03/101449, WO 03/101958, WO 03/104229, WO 03/74500, WO 04/007446, WO 04/007468, WO 04/018467, WO 04/018468, WO 04/018469, WO 04/026822, the teachings of which are herein incorporated by reference in their entirety concerning the inhibitors, their production and their use.

Moreover, WO 03/030946 discloses a gene-therapy for type-2-diabetes by in in vivo expression of glucagon-like peptide (GLP-1) and/or glucose dependent insulinotropic peptide (GIP), optionally in combination with concurrent administration of dipeptidyl peptidase IV (DPP-IV) inhibitors.

All these documents and applications mentioned in this application shall be deemed to be incorporated herein by reference.

Definitions:

The following definitions refer to the whole description and especially to the claims.

The term “alkyl” refers to a saturated, linear or branched, substituted or unsubstituted hydrocarbon group having 1 to 30 carbons atoms, preferably 1 to 20 carbon atoms, more preferably 1, 2, 3, 4, 5, 6, 7, 8, 9, 10, 11, or 12 carbon atoms. Concrete examples for an alkyl group comprise methyl (—CH₃), ethyl (—C₂H₅), n-propyl (n-C₃H₇), iso-propyl (—CH(CH₃)₂), n-butyl (—n-C₄H₉), iso-butyl (—CH₂CH(CH₃)₂), sek-butyl (—CH(CH₃)(C₂H₅)), tert-butyl (—C(CH₃)₃), n-amyl (n-C₅H₁₁), iso-amyl (—(CH₂)₂CH(CH₃)₂), neo-amyl (—CH₂C(CH₃)₃), tert-amyl (—C(CH₃)₂(C₂H₅)), n-hexyl (n-C₆H₁₃), 2,2-dimethyl-butyl (—CH₂C(CH₃)₂(C₂H₅)), iso-hexyl (—(CH₂)₃CH(CH₃)₂), neo-hexyl (—(CH₂)₂C(CH₃)₃), tert-hexyl (—C(CH₃)₂(n-C₃H₇)), n-heptyl (n-C₇H₁₅), iso-heptyl (—(CH₂)₄CH(CH₃)₂), neo-heptyl (—(CH₂)₃C(CH₃)₃), tert-heptyl (—C(CH₃)₂(n-C₄H₉)), n-octyl (n-C₈H₁₇), iso-octyl (—(CH₂)SCH(CH₃)₂)), tert-octyl (—C(CH₃)₂(n-C₅H₁₁)), neo-octyl (—(CH₂)₄C(CH₃)₃) or 2,2,4-trimethyl-pentyl (—CH₂—CH(CH₃)CH₂C(CH₃)₃) group.

The term “alkenyl” refers to an unsaturated, linear or branched, substituted or unsubstituted hydrocarbon group having at least one double bond having 2 to 30 carbons atoms, preferably 2 to 20 carbon atoms, more preferably 2, 3, 4, 5, 6, 7, 8, 9, 10, 11, or 12 carbon atoms. The alkenyl group has one or two or three double bonds, preferably one or two double bonds, and more preferably one double bond. Concrete examples for an alkenyl group comprise vinyl (—CH═CH₂), allyl (—CH₂CH═CH₂), prop-1-enyl (—CH═CHCH₃), but-1-enyl (—CH═CH(C₂H₅)), but-2-en-1-yl (—CH₂CH═CH(CH₃)), but-3-en-1-yl (—(CH₂)₂CH═CH₂), 2-methyl-prop-2-enyl (—CH₂C(═CH₂)(CH₃)), buta-1,3-dien-1-yl (—CH═CH—CH═CH₂), 3-methyl-buta-1,3-dienyl (—CH═CH—C(═CH₂)(CH₃)), isoprenyl (—CH₂—CH═C(CH₃)₂), or hex-2-enyl (—CH₂—CH═CH—C₃H₇) group.

If the formation of an E configuration or, respectively, a Z configuration of a double bond in an “alkenyl group” is possible, both the E and Z configuration are comprised in this application.

The term “alkinyl” refers to a unsaturated, linear or branched, substituted or unsubstituted hydrocarbon group having at least one triple bond having 2 to 30 carbons atoms, preferably 2 to 20 carbon atoms, more preferably 2, 3, 4, 5, 6, 7, 8, 9, 10, 11, or 12 carbon atoms. The alkinyl group has one or two or three triple bonds, preferably one triple bond. Concrete examples for an alkinyl group comprise acetylenyl (—C≡CH), propargyl (—C≡C—CH₃), but-1-in-1-yl (—C≡C—C₂H₅), but-2-in-1-yl (—CH₂—C≡C—CH₃), but-3-in-1-yl (—(CH₂)₂—C≡CH) group.

Generally, the term “alkenyl group” and “alkinyl group” comprises also compounds having double bonds and, additionally, triple bonds, i.e. “alkeninyl groups”, having preferably one double bond and, additionally, one triple bond. As an example therefore, the group 4,7-dimethyl-oct-6-en-2-in-1-yl (—CH₂—C≡C—CH(CH₃)—CH₂—CH═C(CH₃)₂) may be given.

Number of rings: Generally, all the cyclic groups have one, two, three or more rings in the group, preferably one or two rings, more preferably one ring. Two or more rings can be connected by ring annelation, by a single bond or by a spiro atom. This fact also relates, independently of each other, to cycloalkyl, cycloalkenyl, cycloalkinyl, heterocycloalkyl, heterocycloalkenyl, aryl, heteroaryl, as well as to other cyclic groups.

Generally, the terms “alkyl, alkenyl, and alkinyl” refer also to groups, in which one, two, three, four, five or more, preferably three, most preferably one of the hydrogen atoms, independently of each other, are substituted by a halogen atom. The term “halogen atom” comprises a fluorine (—F), chlorine (—Cl), bromine (—Br), iodine (—I), respectively. The preferred halogen atoms for substitution are fluorine and chlorine, especially fluorine. Therefore, the terms alkyl, alkenyl and alkinyl groups refer also, for example, to 2,2,2-trichloro-eth-1-yl (—CH₂CCl₃), trifluoromethyl (—CF₃), 2,2,2-trifluoro-eth-1-yl (—CH₂CF₃) or pentafluoro-ethyl (—CF₂CF₃) group. This kind of substitution also relates to cycloalkyl, cycloalkenyl, cycloalkinyl, heteroalkyl, heteroalkenyl, heterocycloalkyl, heterocycloalkenyl, aryl, heteroaryl, aryl-alkyl, heteroaryl-alkyl, aryl-heteroalkyl, heteroaryl-heteroalkyl groups mentioned below and correspondingly to all other groups mentioned in this application. Further examples therefore are given at the corresponding paragraphes for the definition.

Furthermore, the hydrogen atoms of the alkyl, alkenyl, and alkinyl groups may be further substituted, independently of each other, by hydroxy (—OH), oxo (═O), thiol (—SH), thio (═S), amino (—NH₂), imino (═NH), oder nitro (—NO₂). This kind of substitution also relates to cycloalkyl, cycloalkenyl, cycloalkinyl, heteroalkyl, heteroalkenyl, heterocycloalkyl, heterocycloalkenyl, aryl, heteroaryl, aryl-alkyl, heteroaryl-alkyl, aryl-heteroalkyl, heteroaryl-heteroalkyl groups mentioned below and correspondingly to all other groups mentioned in this application. Examples therefore are given at the corresponding paragraphes for the definition.

Under a “linear alkyl group” an alkyl group with a single straight carbon chain—without a branching point—is understood, which is derived, for example, from “normal-alkanes” or “n-alkanes”.

Examples therefore are n-propyl (—CH₂—CH₂—CH₃), n-butyl (—CH₂—CH₂—CH₂—CH₃) or n-amyl (—CH₂—CH₂—CH₂—CH₂—CH₃).

Under a “branched alkyl group” an alkyl group is understood which has one, two, three or more branching points, preferably one branching point, in the carbon chain of the alkane Branched alkyl groups are derived, for example, from iso-alkanes or neo-alkanes.

Examples therefore are iso-propyl (—CH(CH₃)₂), iso-butyl (—CH₂CH(CH₃)₂), sec-butyl (—CH(CH₃)(CH₂CH₃), tert-butyl (—C(CH₃)₃), or neo-amyl (—CH₂C(CH₃)₃); the branching point is marked in bold type.

These definitions shall be deemed to be valid for all other groups mentioned correspondingly.

The term “cycloalkyl” refers to a saturated, substituted or unsubstituted, cyclic hydrocarbon group having 3 to 30 carbons atoms, preferably 3 to 20 carbon atoms, more preferably 3, 4, 5, 6, 7, 8, 9, 10, 11, or 12 carbon atoms. Preferably, the cycloalkyl group contains 3, 4, 5, 6, 7, 8, 9 or 10 carbon atoms in the ring.

Concrete examples for a substituted or unsubstituted cycloalkyl group comprise cyclopropyl, cyclobutyl, cyclopentyl, 2-methyl-cyclopent-1-yl, 3-methyl-cyclopent-1-yl, cyclohexyl, 2-methyl-cyclohex-1-yl, 3-methyl-cyclohex-1-yl, 4-methyl-cyclohex-1-yl, 4-ethyl-cyclohex-1-yl; 4-isopropyl-cyclohex-1-yl, 3,5-dimethyl-cyclohex-1-yl, cycloheptyl, cyclooctyl; 4-isopropyl-cyclooct-1-yl, (4-cyclopentyl)-cyclohexyl, spiro[4,5]-decanyl, norbornyl, decalinyl, cubanyl, bicyclo[4.3.0.]-nonyl, tetralinyl, or fluoro-cyclohexyl group.

Further examples for a substituted cycloalkyl group are cyclopentan-1-on-2-yl, cyclopentan-1-on-3-yl, cyclohexan-1-on-2-yl, cyclohexan-1-on-3-yl, cyclohexan-1-on-4-yl group.

The term “cycloalkenyl” refers to a partially unsaturated, substituted or unsubstituted, cyclic hydrocarbon group having 3 to 30 carbons atoms, preferably 3 to 20 carbon atoms, more preferably 3, 4, 5, 6, 7, 8, 9, 10, 11, or 12 carbon atoms. Preferably, the cycloalkenyl group contains 3, 4, 5, 6, 7, 8, 9 or 10 carbon atoms in the ring. The cycloalkenyl group has one or two or three double bonds, preferably one or two double bonds, more preferably one double bond; the double may be exocyclic or endocyclic, preferably endocyclic.

Concrete examples for a substituted or unsubstituted cycloalkenyl group comprise cyclopent-1-en-1-yl, 2-methyl-cyclopent-1-en-1-yl, 3-methyl-cyclopent-1-en-1-yl, 4-methyl-cyclopent-1-en-1-yl, 5-methyl-cyclopent-1-en-1-yl, cyclopent-1-en-3-yl, cyclopent-1-en-4-yl, cyclopenta-1,3-dien-5-yl, cyclohex-1-en-1-yl, cyclohex-1-en-3-yl, cyclohex-1-en-4-yl, 2-methyl-cyclohex-1-en-1-yl, 3-methyl-cyclohex-1-en-1-yl, 4-methyl-cyclohex-1-en-1-yl, 5-methyl-cyclohex-1-en-1-yl, 6-methyl-cyclohex-1-en-1-yl, 1-methyl-cyclohex-1-en-3-yl, 2-methyl-cyclohex-1-en-3-yl, 3-methyl-cyclohex-1-en-3-yl, 4-methyl-cyclohex-1-en-3-yl, 5-methyl-cyclohex-1-en-3-yl, 6-methyl-cyclohex-1-en-3-yl, cyclohexa-1,3-dien-1-yl, cyclohexa-1,3-dien-2-yl, cyclohexa-1,3-dien-5-yl, 4-methylen-cyclohex-1-yl, or 4-(propyl-2-en)-cyclohex-1-yl group.

Further examples for a cycloalkenyl group are cyclopent-2-en-1-on-2-yl, cyclopent-2-en-1-on-3-yl, cyclohex-2-en-1-on-2-yl, cyclohex-2-en-1-on-3-yl, cyclohex-2-en-1-on-4-yl.

The term “cycloalkinyl” refers to a partially unsaturated, substituted or unsubstituted, cyclic hydrocarbon group having 6 to 30 carbons atoms, preferably 6 to 20 carbon 7 atoms, more preferably 6, 7, 8, 9, 10, 11, or 12 carbon atoms. Preferably, the cycloalkinyl group contains 6, 7, 8, 9 or 10 carbon atoms in the ring. The cycloalkinyl group has one or two triple bonds, preferably one triple bond. The triple bond may be exocyclic or endocyclic, preferably endocyclic.

Concrete examples are the cyclooct-1-in-3-yl, cyclooct-1-in-4-yl and the cyclooct-1-in-5-yl group.

The terms “heteroalkyl” refers to a saturated, linear or branched, substituted or unsubstituted hydrocarbon group having 1 to 30 carbons atoms, preferably 1 to 20 carbon atoms, more preferably 1, 2, 3, 4, 5, 6, 7, 8, 9, 10, 11, or 12 carbon atoms, wherein one or more carbon atoms, independently of each other, are substituted by nitrogen, oxygen, or sulfur. Generally, one, two or three carbon atoms are substituted by nitrogen, oxygen, or sulfur, preferably one or two, more preferably one.

Furthermore, the term “heteroalkyl” also refers to a carboxylic acid group or a group derived from a carboxylic acid group, as for example acyl, acyl-alkyl, alkoxycarbonyl, acyloxy, acyloxyalkyl, carboxyalkylamid, alkoxycarbonyloxy. Further examples for heteroalkyl groups are nitrile, isonitrile, cyanat, isocyanat, thiocyanat, isothiocyanat, carbonyl in combination with alkyl groups.

Concrete examples for an “heteroalkyl” group comprise methoxy (—OCH₃), hydroxymethyl (—CH₂—OH), carboxy-methyl (—CH₂—COOH), carboxamide-methyl (—CH₂—CO—NH₂), trifluoromethoxy (—OCF₃), ethoxy (—OC₂H₅), hydroxy-ethyl (—CH₂—CH₂—OH), hydroxy-ethoxyl (—O—CH₂—CH₂—OH), amino-ethoxyl (—O—CH₂—CH₂—NH₂), di-N,N-(hydroxy-ethyl)-amino (—N(CH₂—CH₂—OH)₂), n-propoxy (—O-n-C₃H₇), iso-propoxy (—O—CH(CH₃)₂), 2-hydroxy-prop-1-yl (—CH₂—CH(OH)—CH₃), n-butoxy (—O-n-C₄H₁₀), tert-butoxy (—OC(CH₃)₃), methoxy-methyl (—CH₂—O—CH₃), ethoxy-methyl (—CH₂—O—C₂H₅), 2-methoxy-ethyl (—(CH₂)₂—O—CH₃), 2-ethoxy-ethyl (—(CH₂)₂—O—C₂H₅), 2′-hydroxy-2-ethoxy-ethyl (—(CH₂)₂—O—(CH₂)₂—OH), 2′-hydroxy-2-ethoxy-ethoxy (—O—(CH₂)₂—O—(CH₂)₂—OH), enol ethers; or N-methyl-amino (—NH(CH₃)), N,N-dimethylamino (—N(CH₃)₂), N-ethyl-amino (—NH(C₂H₅)), N,N-diethyl-amino (—N(C₂H₅)₂), N-isopropyl-amino (—NH(CH(CH₃)₂)), N-ethyl-N-isopropyl-amino (—N(C₂H₅)(CH(CH₃)₂)), N,N-diisopropyl-amino (—N(CH(CH₃)₂)₂), N-methyl-amino-methyl (—CH₂—NH(CH₃)), N-ethyl-amino-methyl (—CH₂—NH(C₂H₅)), N,N-dimethylamino-methyl (—CH₂—N(CH₃)₂), N,N-diisopropyl-amino-ethyl (—(CH₂)₂—N(CH(CH₃)₂)₂), 2-(N,N-dimethyl-amino)-ethyl (—(CH₂)₂—N(CH₃)₂), 2-(N,N-diethyl-amino)-ethoxy (—O—(CH₂)₂—N(C₂H₅)₂); or methyl-mercapto (—SCH₃), ethyl-mercapto (—SC₂H₅), n-propyl-mercapto (—S-n-C₃H₇), n-butyl-mercapto (—S-n-C₄H₁₀) group; or acetyl (—CO—CH₃), propionyl (—CO—C₂H₅), butyryl (—CO-n-C₃H₇), acetyloxy (—O—CO—CH₃), propionyloxy (—O—CO—C₂H₅), butyryloxy (—O—CO—C₃H₇), methoxy-carbonyl (—CO—OCH₃), ethoxy-carbonyl (—CO—OC₂H₅), 2′-hydroxy-ethoxy-carbonyl (—CO—O—(CH₂)₂—OH), methoxy-carbonyloxy (—O—CO—OCH₃), ethoxy-carbonyloxy (—O—CO—OC₂H₅), dimethylamino-carbonyl (—CO—N(CH₃)₂), N-methyl-N-ethyl-amino-carbonyl (—CO—N(CH₃)(C₂H₅)), di-N,N-(2′-hydroxy-ethyl) amino-carbonyl (—CO—N(CH₂—CH₂—OH)₂), N-methyl-N-ethyl-amino-carbonyloxy (—O—CO—N(CH₃)(C₂H₅)), dimethylamino-carbonyloxy (—O—CO—N(CH₃)₂), ureyl (—NH—CO—NH₂), N,N-dimethyl-ureyl (—NH—CO—N(CH₃)₂) group; or nitrile (—C═N), nitrilo-methyl (—CH₂—C≡N), 2-nitrilo-ethyl (—(CH₂)₂—C≡N), isonitrile (—N═C), isonitrilo-methyl (—CH₂—N═C), cyanat (—O—C≡N), isocyanat (—N═C═O), thiocyanat (—S—C≡N), isothiocyanat (—N═C=S), formyl (—CHO), formyl-methyl (—CH₂—CHO), 2-formyl-ethyl (—(CH₂)₂—CHO) group.

The terms “heteroalkenyl” refers to an unsaturated, linear or branched, substituted or unsubstituted hydrocarbon group having 2 to 30 carbons atoms, preferably 2 to 20 carbon atoms, more preferably 2, 3, 4, 5, 6, 7, 8, 9, 10, 11, or 12 carbon atoms, wherein one or more carbon atoms, independently of each other, are substituted by nitrogen, oxygen, or sulfur. Generally, one, two or three carbon atoms are substituted by nitrogen, oxygen, or sulfur, preferably one or two, more preferably one. The heteroalkenyl group has one or two or three double bonds, preferably one or two double bond, more preferably one double bond. Concrete examples for an heteroalkenyl group comprise allyloxy (—O—CH₂CH═CH₂), 2-methyl-prop-2-enyl-1-oxy (—O—CH₂C(CH₃)═CH₂), allylamino (—NH(CH₂CH═CH₂)), N,N-diallylamino (—N(CH₂CH═CH₂)₂) group.

The term “heterocycloalkyl” refers to a saturated, substituted or unsubstituted, cyclic hydrocarbon group having 1 to 30 carbons atoms, preferably 1 to 20 carbon atoms, more preferably 1, 2, 3, 4, 5, 6, 7, 8, 9, 10, 11, or 12 carbon atoms. Preferably, the heterocycloalkyl group contains 1, 2, 3, 4, 5, 6, 7, 8, 9 or 10 carbon atoms in the ring, wherein one, two, three or more ring carbon atoms, independently of each other, are substituted by nitrogen, oxygen, or sulfur. Generally, one, two or three ring carbon atoms are substituted by nitrogen, oxygen, or sulfur, preferably one or two, more preferably one. The hetero atoms may be a part of the ring or substituents attached to the ring, preferably they are a part of the ring.

Concrete examples of a heterocycloalkyl group comprise a substituted or unsubstituted oxirano, aziridino, oxacyclopropyl, azacyclopropyl, thiirano, oxetano, thietano, pyrrolidino, tetrahydrofurano, thiolano, 1,1-dioxo-thiolano, 1,3-dioxolano, thiazolidino, imidazolidino, oxazolidino, pyrazolidino, tetrahydropyrano, piperidino, urotropino, piperazino, N-methyl-piperazino, (2-(N-methyl)-N′-piperazinyl)-ethyl, (4N-(2′-hydroxyethyl)-1N-piperazinyl), (2-(4N-(2′-hydroxyethyl)-1N-piperazinyl)-ethyloxy), morpholino, 2-(N-morpholino)-ethyl group, as well as lactames, lactones, cyclic imides and cyclic anhydrides.

The term “heterocycloalkenyl” refers to an unsaturated, substituted or unsubstituted, cyclic hydrocarbon group having 2 to 30 carbons atoms, preferably 2 to 20 carbon atoms, more 30 preferably 2, 3, 4, 5, 6, 7, 8, 9, 10, 11, or 12 carbon atoms. Preferably, the heterocycloalkenyl group contains 2, 3, 4, 5, 6, 7, 8, 9 or 10 carbon atoms in the ring, wherein one or more ring carbon atoms, independently of each other, are substituted by nitrogen, oxygen, or sulfur.

Generally, one, two or three ring carbon atoms are substituted by nitrogen, oxygen, or sulfur, preferably one or two, more preferably one. The hetero atoms may be a part of the ring or substituents attached to the ring, preferably they are a part of the ring. The heterocycloalkenyl group has one or two or three double bonds, preferably one or two double bonds, more preferably one double bond; the double may be exocyclic or endocyclic, preferably endocyclic.

Concrete examples of a heterocycloalkyl group comprise substituted or unsubstituted pyrrolinyl, 2,3-dihydrofuranyl, 2,5-dihydrofuranyl, 2,3-dihydrothiophenyl, 1,1-dioxo-2,5-dihydro-thiophenyl, 2,5-dihydrothiophenyl, thiazolinyl, imidazolinyl, oxazolinyl, pyrazolinyl group.

The term “aryl” refers to a carbocyclic, aromatic, substituted or unsubstituted hydrocarbon group having 5 to 30 carbons atoms, preferably 5 to 20 carbon atoms, more preferably 5, 6, 7, 8, 9, 10, 11, or 12 carbon atoms. The aryl group has generally one, two, three or more rings, preferably one or two rings, more preferably one ring, wherein the rings may be connected by annellation or by a single bond. Generally, the aryl group has 5, 6, 7, 8, 9, 10, 11, 12, 13, or 14 ring carbon atoms, preferably 6, 7, 8, 9, or 10 ring carbon atoms, more preferably 6 ring carbon atoms.

Concrete examples for a substituted or unsubstituted aryl group comprise substituted or unsubstituted phenyl, 4-fluoro-phenyl, 3-fluoro-phenyl, pentafluoro-phenyl, 4-hydroxyphenyl, 3-nitro-phenyl, 4-(trifluoromethyl)-phenyl, 4-anilinyl, 2-biphenylyl, 3-biphenylyl, 4-biphenylyl, indenyl, 1-naphthyl, or 2-naphthyl, 1-anthracenyl, 2-anthracenyl, 3-anthracenyl, group.

The term “heteroaryl” refers to a aromatic, substituted or unsubstituted hydrocarbon group having 1 to 30 carbons atoms, preferably 1 to 20 carbon atoms, more preferably 1, 2, 3, 4, 5, 6, 7, 8, 9, 10, 11, or 12 carbon atoms, and, furthermore, the heteroaryl group has 1, 2, 3, 4, 5, or 6 hetero atoms, preferably 1, 2, 3, or 4, more preferably 1, 2 or 3 hetero atoms, further more preferably 1 or 2 hetero atoms and, most preferably 1 hetero atom, which are independently of each other selected from oxygen, nitrogen and sulfur. The hetero atoms may be a part of the ring or a part of the substituent, preferably, they are a part of the ring. The aryl group has generally one, two, three or more rings, preferably one or two rings, more preferably one ring, wherein the rings may be connected by annellation or by a single bond. Generally, the heteroaryl group has 1, 2, 3, 4, 5, 6, 7, 8, 9, 10, 11, 12, 13, or 14 ring carbon atoms, preferably 1, 2, 3, 4, 5, 6, 7, 8, 9, or 10 ring carbon atoms, as well as 1, 2, 3, 4, or 5 ring heteroatoms, preferably 1, 2, or 3 ring heteroatoms, further more preferably 1 or 2 ring heteroatoms, most preferably 1 ring heteroatom.

Concrete examples for a substituted or unsubstituted heteroaryl group comprise substituted or unsubstituted furanyl, thiophenyl, pyrrolyl, oxazolyl, thiazolyl, 1-imidazolyl, 2-imidazolyl, 4-imidazolyl, 3-phenyl-1-pyrrolyl, isoxazolyl, isothiazolyl, 3-pyrazolyl, 1,2,3-triazolyl, 1,2,4-triazolyl, tetrazolyl, 4-pyridinyl, 3-pyridinyl, 2-pyridinyl, pyridazinyl, pyrimidinyl, pyrazinyl, indazolyl, 6-indolyl, benzimidazolyl, chinolinyl, isochinolinyl, purinyl, carbazolinyl, acridinyl, and 2,3′-bifuryl group.

The term “aryl-alkyl” refers to an aryl group as defined above and an alkyl group as defined above. Therefore, an aryl-alkyl group has at least one, two or more substituted or unsubstituted aryl groups, preferable one or two aryl groups, more preferably one aryl group, as defined above, and further, one, two or more substituted or unsubstituted alkyl groups, preferable one or two alkyl groups, more preferably one alkyl group, as defined above.

Concrete examples for a substituted or unsubstituted aryl-alkyl group comprise substituted or unsubstituted benzyl, 2-phenyleth-1-yl, p-tolyl-methyl, p-tolyl-ethyl, 2-(4-ethyl-phenyl)-eth-1-yl group p-tolyl, m-tolyl, o-tolyl, 2,3-dimethyl-phenyl, 2,4-dimethyl-phenyl, 2,5-dimethyl-phenyl, 2,6-dimethyl-phenyl, 3,4-dimethyl-phenyl, 3,5-dimethyl-phenyl, 2,4,6-trimethyl-phenyl, benzhydryl (=diphenyl-methyl), trityl (=triphenyl-methyl), α-styryl, β-styryl, cumyl, 2-ethyl-phenyl, 3-ethyl-phenyl, 4-ethyl-phenyl, 2-fluoro-benzyl, 1-methyl-2-fluoro-phen-6-yl, 1-methyl-2-fluoro-phen-4-yl, 1H-indenyl, indanyl, indanl-on-2-yl, tetralinyl, fluorenyl, (3-phenyl)-cyclopent-1-yl, dihydronaphthalinyl, or (4-cyclohexyl)-phenyl, group.

The term “heteroaryl-alkyl” refers to an heteroaryl group as defined above, and an alkyl group as defined above. Therefore, an aryl-alkyl group has at least one, two or more substituted or unsubstituted heteroaryl groups, preferably one or two heteroaryl groups, more preferably one heteroaryl group, as defined above, and further, one, two or more substituted or unsubstituted alkyl groups, preferable one or two alkyl groups, more preferably one alkyl group, as defined above.

Concrete examples for a substituted or unsubstituted heteroaryl-alkyl group comprise substituted or unsubstituted N-methyl-pyrrol-2-yl, N-methyl-pyrrol-3-yl, 2-methyl-pyrrol-1-yl, (2-methyl-pyrrol-1-yl)-methyl, 3-methyl-pyrrol-1-yl, 4-pyridino-methyl, 4-pyridino-ethyl, 2-(thiazol-2-yl)-ethyl, tetrahydroisochinolinyl, 2-ethyl-indol-1-yl, 3-ethyl-indol-1-yl, 4-methyl-pyridin-2-yl, 4-methyl-pyridin-3-yl, group.

The term “aryl-heteroalkyl” refers to an aryl group as defined above and a heteroalkyl group as defined above. Therefore, an aryl-heteroalkyl group has at least one, two or more substituted or unsubstituted aryl groups, preferable one or two aryl groups, more preferably one aryl group, as defined above, and further, one, two or more substituted or unsubstituted heteroalkyl groups, preferable one or two heteroalkyl groups, more preferably one heteroalkyl group, as defined above.

Concrete examples for a substituted or unsubstituted aryl-heteroalkyl group comprise phenoxy, phenylamino, diphenylamino, benzyloxy, dibenzylamino, 2-methoxy-phenyl, 3-methoxy-phenyl, 4-methoxy-phenyl, 4-ethoxy-phenyl, 2-phenylethyloxy, 2-phenylethylamino or (2-(4-dimethylamino)-phenyl)-eth-1-oxy, (4-carboxyphenyl) alkyl group, benzoyl (—CO—C₆H₅), phenylacetyl (—CO—CH₂—C₆H₅), phenacyl (—CH₂—CO—C₆H₅) group.

The term “heteroaryl-heteroalkyl” refers to a heteroaryl group as defined above and a heteroalkyl group as defined above. Therefore, a heteroaryl-heteroalkyl group has at least one, two or more substituted or unsubstituted heteroaryl groups, preferably one or two heteroaryl groups, more preferably one heteroaryl group, as defined above, and further, one, two or more substituted or unsubstituted heteroalkyl groups, preferably one or two heteroalkyl groups, more preferably one heteroalkyl group, as defined above.

Concrete examples for a substituted or unsubstituted heteroaryl-heteroalkyl group comprise substituted or unsubstituted 2-(4-pyridino-ethyl)-amino, 2-(4-pyridino-methyl)oxy, 2-(2-thiazolo-ethyl)-amino group.

Combinations: Also within the scope of the present invention are combinations of two, three or more groups, preferably two groups listed above, which are not mentioned explicitely, for example aryl-heteroaryl, heterocycloalkyl-aryl, cycloalkyl-aryl, heterocycloalkyl-heteroaryl, cycloalkenyl-heteroaryl, heterocycloalkenyl-aryl, etc.

Concrete examples therefore are 4-phenyl-cyclohex-1-yl, 4-phenyl-cyclohex-1-en-1-yl, 4-(2-pyridinyl)-cyclohex-1-yl, 4-(2-pyridinyl)-cyclohex-1-en-1-yl, 4N-phenyl-piperazin-1N-yl, 5-phenyl-1H-tetrazol-1-yl, 4N-(2-(5-phenyl)-thiazolyl)-piperazin-1N-yl group.

The term “halogen” comprises fluorine (—F), chlorine (—Cl), bromine (—Br), and iodine (—I), respectively.

The term “electron withdrawing group” refers to a atom with a high electronegativity on the Pauling scale or a comparable group capable of withdrawing electrons, like groups having a double or triple bound and having hetero atoms like nitrogen, oxygen and sulfur; The term “electron withdrawing group” comprises further two single bound atoms or one double bound atom. Examples for an “electron withdrawing group” are the halogen atoms fluorine (—F), chlorine (—Cl), bromine (—Br), iodine (—I), and the double bound oxygen atom (═O). As an example for the “electron withdrawing group”, the cyano group (—C≡N) may be given. Preferred as an “electron withdrawing group” are two single bound fluorine atoms (—F)₂ and the double bound oxygen atom (═O), especially preferred is the the double bound oxygen atom (═O).

Glutamine: Throughout the description the expression “glutamine” or “glutaminyl”, respectively, should be considered in that “homoglutamine” or “homoglutaminyl”, respectively, is also comprised within this wording, i.e., the amino acids mentioned above may have L and D configuration in the Fischer projection, as well as an amino group in α or β position of the carbon chain. Preferably the wording “glutamine” or “glutaminyl” comprises the group L-α-glutamine (—CO—CH(NH₂)—(CH₂)₂—CO—NH₂), L-α-homoglutamine (—CO—CH(NH₂)—(CH₂)₃—CO—NH₂), and L-β-homoglutamine (—CO—CH₂—CH(NH₂)—(CH₂)₂—CO—NH₂), most preferably L-α-glutamine.

Stereoisomers:

All possible stereoisomers of the claimed compounds are included in the present invention. Especially preferred for the glutamine group are the L-α-glutamine (—CO—CH(NH₂)—(CH₂)₂—CO—NH₂), L-α-homoglutamine (—CO—CH(NH₂)—(CH₂)₃—CO—NH₂), and L-β-homoglutamine (—CO—CH₂—CH(NH₂)—(CH₂)₂—CO—NH₂) group, most preferred is the L-α-glutamine group.

Concerning the stereoisomers of the prolin mimetica, all possible stereoisomers of the compounds having proline mimetica of the structural formulas (II) to (IX) of the present invention are included in this application. Especially, that configuration at the “a carbon atom” of the prolin-mimetica of the structural formulas (II) to (IX) of the present invention is preferred, which imitates the stereochemical configuration of the naturally occuring amino acid L-α-proline at its a carbon atom. Therefore, prolin mimetica of the structural formulas (II) to (IX) of the present invention have preferably that sterochemical configuration at the “a carbon atom”, which corresponds to the stereochemical configuration of L-α-proline at its a carbon atom.

Naturally occurring L-α-proline has an absolute S-configuration at its α-carbon atom in the sense of the Cahn-Ingold-Prelog nomenclature. If the carboxylic acid group of L-α-proline is imitated by the cyano, 2H-tetrazol-5-yl, or phosphonic acid diphenyl ester group, the preferred configuration will be the S configuration at the α carbon atom of the prolin mimeticum of the structural formulas (II) to (IX) of the present invention; in the case that the —COOH group of prolin is imitated by a boronic acid group, the absolute configuration at the α carbon atom of the prolin mimeticum of the structural formulas ([1) to (IX) of the present invention will change to R due to the lower molecular mass of a boron atom compared with a carbon atom. Despite the fact that the absolute configuration of the α carbon atom of the proline mimetica of the structural formulas (II) to (IX) of the present invention may change due to the change of the substituents of the α carbon atom, the absolute configuration at the α carbon atom corresponding to that of the naturally occurring amino acid L-α-proline is always preferred.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Also comprised within the present invention are all possible stereoisomers of compounds with proline mimetica having stereochemical centers other than that which corresponds to the α carbon atom of the L-α-proline.

Preparation and Isolation of Stereoisomers:

Where the processes for the preparation of the compounds according to the invention give rise to a mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their components enantiomers by standard techniques, such as the formation of diastereomeric pairs by salt formation with an optically active acid, such as (−)-di-p-toluoyl-d-tartaric acid and/or (+)-di-p-toluoyl-1-tartaric acid followed by fractional crystallization and regeneration of the free base. The compounds may also resolved by formation of diastereomeric esters or amides, followed by chromatographic separation and removal of the chiral auxiliary. Alternatively, the compounds may be resolved using a chiral HPLC column.

Pharmaceutically Acceptable Salts:

In view of the close relationship between the free compounds and the compounds in the form of their salts, whenever a compound is referred to in this context, a corresponding salt is also intended, provided such is possible or appropriate under the circumstances.

The pharmaceutically acceptable salt generally takes a form in which an amino acids basic side chain is protonated with an inorganic or organic acid. Representative organic or inorganic acids include hydrochloric, hydrobromic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toulenesulfonic, cyclohexanesulfamic, salicylic, saccharinic or trifluoroacetic acid. All pharmaceutically acceptable acid addition salt forms of the compounds of the present invention are intended to be embraced by the scope of this invention.

Polymorph Crystal Forms:

Furthermore, some of the crystalline forms of the compounds may exist as polymorphs and as such are included in the present invention. In addition, some of the compounds may form solvates with water (i.e. hydrates) or common organic solvents, and such solvates are also encompassed within the scope of this invention. The compounds, including their salts, can also be obtained in the form of their hydrates, or include other solvents used for their crystallization, which are also encompassed within the scope of this invention.

Prodrugs:

The present invention further includes within its scope prodrugs of the compounds of this invention. In general, such prodrugs will be functional derivatives of the compounds which are readily convertible in vivo into the desired therapeutically active compound. Thus, in these cases, the methods of treatment of the present invention, the term “administering” shall encompass the treatment of the various disorders described with prodrug versions of one or more of the claimed compounds, but which converts to the above specified compound in vivo after administration to the subject. Conventional procedures for the selection and preparation of suitable prodrug derivatives are described, for example, in “Design of Prodrugs”, ed. H. Bundgaard, Elsevier, 1985 and the patent applications DE 198 28 113, DE 198 28 114, WO 99/67228 and WO 99/67279 which are fully incorporated herein by reference.

Protective Groups:

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991, fully incorporated herein by reference. The protecting groups may be removed at a convenient subsequent stage using methods known from the art.

Amino Acids

Examples of amino acids which can be used in the present invention are L and D-amino acids, N-methyl-amino acids, aza-amino acids; allo- and threo-forms of Ile and Thr, which can, e.g. be α-, β- or o)amino acids, whereof α-amino acids are preferred.

Examples of Amino Acids are:

aspartic acid (Asp), glutamic acid (Glu), arginine (Arg), lysine (Lys), histidine (His), glycine (Gly), serine (Ser), cysteine (Cys), threonine (Thr), asparagine (Asn), glutamine (Gln), tyrosine (Tyr), alanine (Ala), proline (Pro), valine (Val), isoleucine (Ile), leucine (Leu), methionine (Met), phenylalanine (Phe), tryptophan (Trp), hydroxyproline (Hyp), beta-alanine (beta-Ala), 2-aminooctanoic acid (Aoa), acetidine-(2)-carboxylic acid (Ace), pipecolic acid (Pip), 3-aminopropionic acid, 4-aminobutyric acid and so forth, alphα-aminoisobutyric acid (Aib), sarcosine (Sar), ornithine (Orn), citrulline (Cit), homoarginine (Har), t-butylalanine (t-butyl-Ala), t-butylglycine (t-butyl-Gly), N-methylisoleucine (N-MeIle), phenylglycine (Phg), cyclohexylalanine (Cha), norleucine (Nle), cysteic acid (Cya) and methionine sulfoxide (MSO), acetyl-Lys, modified amino acids such as phosphoryl-serine (Ser(P)), benzyl-serine (Ser(Bzl)) and phosphoryl-tyrosine (Tyr(P)), 2-aminobutyric acid (Abu), aminoethylcysteine (AECys), carboxymethylcysteine (Cmc), dehydroalanine (Dha), dehydroamino-2-butyric acid (Dhb), carboxyglutaminic acid (Gla), homoserine (Hse), hydroxylysine (Hyl), cis-hydroxyproline (cis Hyp), trans-hydroxyproline (transHyp), isovaline (Iva), pyroglutamic acid (Pyr), norvaline (Nva), 2-aminobenzoic acid (2-Abz), 3-aminobenzoic acid (3-Abz), 4-aminobenzoic acid (4-Abz), 4-(aminomethyl)benzoic acid (Amb), 4-(aminomethyl)cyclohexanecarboxylic acid (4-Amc), Penicillamine (Pen), 2-amino-4-cyanobutyric acid (Cba), cycloalkane-carboxylic aicds.

Examples of m-amino acids are e.g.: 5-Ara (aminoraleric acid), 6-Ahx (aminohexanoic acid), 8-Aoc (aminooctanoic aicd), 9-Anc (aminovanoic aicd), 10-Adc (aminodecanoic acid), 11-Aun (aminoundecanoic acid), 12-Ado (aminododecanoic acid). Further amino acids are: indanylglycine (Igl), indoline-2-carboxylic acid (Idc), octahydroindole-2-carboxylic acid (Oic), diaminopropionic acid (Dpr), diaminobutyric acid (Dbu), naphtylalanine (1-Nal) and (2-NaI), 4-aminophenylalanine (Phe(4-NH₂)), 4-benzoylphenylalanine (Bpa), diphenylalanine (Dip), 4-bromophenylalanine (Phe(4-Br)), 2-chlorophenylalanine (Phe(2-Cl)), 3-chlorophenylalanine (Phe(3-Cl)), 4-chlorophenylalanine (Phe(4-Cl)), 3,4-chlorophenylalanine (Phe (3,4-C₁₂)), 3-fluorophenylalanine (Phe(3-F)), 4-fluorophenylalanine (Phe(4-F)), 3,4-fluorophenylalanine (Phe(3,4-F₂)), pentafluorophenylalanine (Phe(F₅)), 4-guanidinophenylalanine (Phe(4-guanidino)), homophenylalanine (hPhe), 3-jodophenylalanine (Phe(3-J)), 4-jodophenylalanine (Phe(4-J)), 4-methylphenylalanine (Phe(4-Me)), 4-nitrophenylalanine (Phe-4-NO₂)), biphenylalanine (Bip), 4-phosphonomethylphenylalanine (Pmp), cyclohexylglycine (Ghg), 3-pyridinylalanine (3-Pal), 4-pyridinylalanine (4-Pal), 3,4-dehydroproline (A-Pro), 4-ketoproline (Pro(4-keto)), thioproline (Thz), isonipecotic acid (Inp), 1,2,3,4,-tetrahydroisoquinolin-3-carboxylic acid (Tic), propargylglycine (Pra), 6-hydroxynorleucine (NU(6-OH)), homotyrosine (hTyr), 3-jodotyrosine (Tyr(3-J)), 3,5-dijodotyrosine (Tyr(3,5-J₂)), methyltyrosine (Tyr(Me)), 2′,6′-dimethyltyrosine (Dmt), 3-NO₂-tyrosine (Tyr(3-NO₂)), phosphotyrosine (Tyr(PO₃H₂)), alkylglycine, 1-aminoindane-1-carboxylic acid, 2-aminoindane-2-carboxylic acid (Aic), 4-amino-methylpyrrol-2-carboxylic acid (Py), 4-amino-pyrrolidine-2-carboxylic acid (Abpc), 2-aminotetraline-2-carboxylic acid (Atc), diaminoacetic acid (Gly(NH₂)), diaminobutyric acid (Dab), 1,3-dihydro-2H-isoinole-carboxylic acid (Disc), homocylcohexylalanine (hCha), homophenylalanine (hPhe or Hof), trans-3-phenyl-azetidine-2-carboxylic acid, 4-phenyl-pyrrolidine-2-carboxylic acid, 5-phenyl-pyrrolidine-2-carboxylic acid, 3-pyridylalanine (3-Pya), 4-pyridylalanine (4-Pya), styrylalanine, tetrahydroisoquinoline-1-carboxylic acid (Tiq), 1,2,3,4-tetrahydronorharmane-3-carboxylic acid (Tpi), B-(2-thienryl)-alanine (Tha).

“Peptides” are selected from dipeptides to decapeptides, preferred are dipeptides, tripeptides, tetrapeptides and pentapeptides. The amino acids for the formation of the “peptides” can be selected from those listed above.

An “aza-amino acid” is defined as an amino acid where the chiral α-CH group is replaced by a nitrogen atom, whereas an “aza-peptide” is defined as a peptide, in which the chiral □-CH group of one or more amino acid residues in the peptide chain is replaced by a nitrogen atom.

Other amino acid substitutions for those encoded in the genetic code can also be included in peptide compounds within the scope of the invention and can be classified within this general scheme. Proteinogenic amino acids are defined as natural protein-derived α-amino acids. Non-proteinogenic amino acids are defined as all other amino acids, which are not building blocks of common natural proteins. “Peptide mimetics” per se are known to a person skilled in the art. They are preferably defined as compounds which have a secondary structure like a peptide and optionally further structural characteristics; their mode of action is largely similar or identical to the mode of action of the native peptide; however, their activity (e.g. as an antagonist or inhibitor) can be modified as compared with the native peptide, especially vis àvis receptors or enzymes. Moreover, they can imitate the effect of the native peptide (agonist). Examples of peptide mimetics are scaffold mimetics, non-peptidic mimetics, peptoides, peptide nucleic acids, oligopyrrolinones, vinylogpeptides and oligocarbamates. For the definitions of these peptide mimetics see Lexikon der Chemie, Spektrum Akademischer Verlag Heidelberg, Berlin, 1999.

The aim for using these mimetic structures is increasing the activity, increasing the selectivity to decrease side effects, protect the compound against enzymatic degradation for prolongation of the effect.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human, being sought by a researcher, veterinarian, medical doctor or other clinician, which includes alleviation of the symptoms of the disease or disorder being treated.

As used herein, the term “composition” is intended to encompass a product comprising the claimed compounds in the therapeutically effective amounts, as well as any product which results, directly or indirectly, from combinations of the claimed compounds.

Carriers and Additives for Galenic Formulations: Thus, for liquid oral preparations, such as for example, suspensions, elixirs and solutions, suitable carriers and additives may advantageously include water, glycols, oils, alcohols, flavoring agents, preservatives, coloring agents and the like; for solid oral preparations such as, for example, powders, capsules, gelcaps and tablets, suitable carriers and additives include starches, sugars, diluents, granulating agents, lubricants, binders, disintegrating agents and the like.

Carriers, which can be added to the mixture, include necessary and inert pharmaceutical excipients, including, but not limited to, suitable binders, suspending agents, lubricants, flavorants, sweeteners, preservatives, coatings, disintegrating agents, dyes and coloring agents.

Soluble polymers as targetable drug carriers can include polyvinylpyrrolidone, pyran copolymer, polyhydroxypropylmethacrylamidephenol, polyhydroxyethylaspartamide-phenol, or polyethyleneoxidepolyllysine substituted with palmitoyl residue. Furthermore, the compounds of the present invention may be coupled to a class of biodegradable polymers useful in achieving controlled release of a drug, for example, polyactic acid, polyepsilon caprolactone, polyhydroxy butyeric acid, polyorthoesters, polyacetals, polydihydropyrans, polycyanoacrylates and cross-linked or amphipathic block copolymers of hydrogels.

Suitable binders include, without limitation, starch, gelatin, natural sugars such as glucose or betalactose, corn sweeteners, natural and synthetic gums such as acacia, tragacanth or sodium oleate, sodium stearate, magnesium stearate, sodium benzoate, sodium acetate, sodium chloride and the like.

Disintegrators include, without limitation, starch, methyl cellulose, agar, bentonite, xanthan gum and the like.

Indications:

The term “indications” comprises the following diseases, respectively, the following diseases in mammals, preferably humans, can be treated by the compounds of the present invention:

metabolic diseases like impaired glucose tolerance, glucosuria, hyperlipidemia, metabolic acidosis, diabetes mellitus, non-insulin dependent diabetes mellitus, diabetic neuropathy and nephropathy and of sequelae caused by diabetes mellitus;

neurodegenerative diseases; high blood pressure and disturbance of signal action at the cells of the islets of Langerhans and insulin sensitivity in the peripheral tissue in the postprandial phase; the metabolism-related hypertension and cardiovascular sequelae caused by hypertension;

dermal diseases like skin diseases and diseases of the mucosae;

immune and autoimmune disorders, multiple sclerosis, and inflammatory conditions; arthritis; obesity; allograft transplantation; cancer;

neuronal disorders as well as psychosomatic, neuropsychiatric and depressive illnesses, such as anxiety, depression, sleep disorders, chronic fatigue, schizophrenia, epilepsy, nutritional disorders, spasm and chronic pain.

The indications above refer each to both acute and chronic form of the disease.

Further, the following diseases can be treated by the compounds of the present invention:

hyperlipidemia, metabolic acidosis, diabetic neuropathy and nephropathy and of sequelae caused by diabetes mellitus in mammals; metabolism-related hypertension and cardiovascular sequelae caused by hypertension in mammals; for the prohylaxis or treatment of skin diseases and diseases of the mucosae, autoimmune diseases and inflammatory conditions, and for the prophylaxis or treatment of psychosomatic, neuropsychiatric and depressive illness, and neurodegenerative diseases such as anxiety, depression, sleep disorders, chronic fatigue, schizophrenia, epilepsy, nutritional disorders, spasm, and chronic pain, and a simple method for the treatment of those disorders.

Most preferably, the following diseases can be treated by the compounds of the present invention: prediabetes, characterized by IGT, IFG or IGM, diabetes mellitus, preferably non-insulin-dependent diabetes mellitus (type 2 diabetes mellitus) and obesity.

Classification of Diabetes

The newly revised classification of diabetes mellitus is summarized in Table 1. Clinical diabetes may be divided into four general subclasses, including (1) type 1 (caused by beta cell destruction and characterized by absolute insulin deficiency) (2) type 2 (characterized by insulin resistance and relative insulin deficiency (3) other specific types of diabetes (associated with various identifiable clinical conditions or syndromes) and (4) gestational diabetes mellitus. In addition to these clinical categories, two conditions—impaired glucose tolerance and impaired fasting glucose—refer to a metabolic state intermediate between normal glucose homeostasis and overt diabetes. These conditions significantly increase the later risk of diabetes mellitus and may in some instances be part of its natural history. It should be noted that patients with any form of diabetes might require insulin treatment at some point. For this reason the previously used terms insulin-dependent diabetes (for type 1 diabetes mellitus) and non-insulin-dependent diabetes (for type 2) have been eliminated.

Table 1. Classification of Diabetes

Clinical Diabetes

1. Type 1 diabetes, formerly called insulin-dependent diabetes mellitus (IDDM) or “juvenile-onset diabetes”

2. Type 2 diabetes, formerly called non-insulin-dependent diabetes (NIDDM) or “adult-onset diabetes”

3. Other specific types

a) Genetic defects of β-cell function (e.g., maturity-onset diabetes of the young [MODY] types 1-3 and point mutations in mitochondrial DNA)

b) Genetic defects in insulin action

c) Disease of the exocrine pancreas (e.g., pancreatitis, trauma, pancreatectomy, neoplasia, cystic fibrosis, hemochromatosis, fibrocalculous pancreatopathy).

d) Endocrinopathies (e.g. acromegaly, Cusing's syndrome, hyperthyroidism, pheochromocytoma, glucagonoma, somatostinoma, aldosteronoma)

e) Drug or chemical induced (e.g., glucocorticosteroids, thiazides, diazoxide, pentamidine, vacor, thyroid hormone, phenyloin [Dilantin], β-agonists, oral contraceptives)

f) Infections (e.g., congenital rubella, cytomegalovirus)

g) Uncommon forms of immune-mediated diabetes (e.g., “stiff-man”, syndrome, anti-insulin receptor antibodies)

h) Other genetic syndromes (e.g., Down, Klinefelter's, Turner's syndrome, Huntington's disease, myotonic dystrophy, lipodystrophy, ataxia-telangiectasia)

4. Gestational diabetes mellitus

Risk categories

1. Impaired fasting glucose

2. Impaired glucose tolerance

Type 1 Diabetes

Patients with this disorder have little or no insulin secretory capacity and depend on exogenous insulin to prevent metabolic decompensation (e.g., ketoacidosis) and death.

Commonly but not always, diabetes appears abrubtly (i.e., over days and weeks) in previously healthy non-obese children or young adults; in older age groups it may have a more gradual onset. At the time of initial evaluation the typical patient often appears ill, has marked symptoms (e.g., polyuria, polydipsia, polyhagia, and weight loss), and may demonstrate ketoacidosis. Type 1 diabetes is believed to have a long asymptomatic preclinical stage often lasting years, during which pancreatic beta cells are gradually destroyed by an autoimmune attack that is influenced by HLA and other genetic factors, as well as the environment. Initially, insulin therapy is essential to restore metabolism toward normal. However, a so-called honeymoon period may follow and last weeks or moths, during which time smaller doses of insulin are required because of partial recovery of beta cell function and reversal of insulin resistance caused by acute illness.

Thereafter, insulin secretory capacity is gradually lost (over several years). The association of type I diabetes with specific immune response (HLA) genes and the presence of antibodies to islet cells and their constituents provides strong support for the theory that type I diabetes is an autoimmune disease. This syndrome accounts for lese than 10% of diabetes in United States.

Type 2 Diabetes Mellitus

Type 2, by far the most common form of the disease, is found in over 90% of the diabetic patient population. These patients retain a significant level of endogenous insulin secretory capacity. However, insulin levels are low relative to the magnitude of insulin resistance and ambient glucose levels. Type 2 patients are not dependent on insulin for immediate survival and ketosis rarely develops, except under conditions of great physical stress. Nevertheless, these patients may require insulin therapy to control hyperlgycemia. Type 2 diabetes typically appears after the age of 40 years, has a high rate of genetic penetrance unrelated to HLA genes, and is associated with obesity. The clinical features of type 2 diabetes may be mild (fatigue, weakness, dizziness, blurred vision, or other non-specific complaints may dominate the picture) or may be tolerated for many years before the patient seeks medical attention. Moreover, if the level of hyperglycemia is insufficient to produce symptoms, the disease may become evident only after complications develop.

Other Specific Types of Diabetes

This category encompasses a variety of diabetic syndromes attributed to a specific disease, drug, or condition. Genetic research has provided new insights into pathogenesis of MODY, which was formerly included as a form of type 2 diabetes. MODY encompasses several genetic defects of beta cell function, among which mutations at several genetic loci on different chromosomes have been identified. The most common forms—MODY type 3—is associated with a mutation for a transcription factor encoded on chromosome 12 named hepatocyte nuclear 1α (HNF 1, also known as TCF1) and—MODY type 2 is associated with mutations of the glucokinase gene (on chromosome 7) Mutations of the HNF-4α gene (on chromosome 20) are responsible for type 1 of MODY. Each of these conditions is inherited in an autosomal dominant pattern. Two new rare forms of MODY are associated with mutations of the HNF-1β (on chromosome 17) and an insulin gene transcription factor termed PDX-1 or 1DX-1 (on chromosome 13).

The distinction between the various subclasses of diabetes mellitus is usually made on clinical grounds. However, a small subgroup of patients are difficult to classify, that is, they display features common to both type 1 and 2 diabetes. Such patients are commonly non-obese and have reduced insulin secretory capacity that is not sufficient to make them ketosis prone. Many initially respond to oral agents but, with time, require insulin. Some appear to have a slowly evolving form of type 1 diabetes, whereas others defy easy categorization.

Gestational Diabetes

The term gestational diabetes describes women with impaired glucose tolereance that appears or is first detected during pregnancy. Gestational diabetes usually appears in the 2^{nd} or 3^{rd} trimester, a time when pregnancy-associated insulin antagonistic hormones peak. After delivery, glucose tolerance generally (but not always) reverts to normal.

Diagnosis

The diagnosis of diabetes is usually straightforward when the classic symptoms of polyuria, polydipsia, and weight loss are present. All that is required is a random plasma glucose measurement from venous blood that is 200 mg/dL or greater. If diabetes is suspected but not confirmed by a random glucose determination, the screening test of choice is overnight fasting plasma glucose level. The diagnosis is established if fasting is equal to or greater than 126 mg/dL on at least two separate occasions.

Related Conditions

Impaired Glucose Tolerance and Impaired Fasting Glucose

Impaired glucose tolerance (IGT) and impaired fasting glucose (IFG) are terms applied to individuals who have glucose levels that are higher than normal, (under fed or fasting conditions, respectively) but lower than those accepted as diagnostic for diabetes mellitus. Both conditions are associated with an increased risk for cardiovascular disease, but do not produce the classic symptoms or the microvascular and neuropathic complications associated with diabetes mellitus.

In a subgroup of patients (about 25 to 30%), however, type 2 diabetes eventually develops.

Impaired Glucose Metabolism

Impaired Glucose Metabolism (IGM) is defined by blood glucose levels that are above the normal range but are high enough to meet the diagnostic criteria for type 2 diabetes mellitus. The incidence of IGM varies from country to country, but usually occurs 2-3 time more frequently than overt diabetes. Until recently, individuals with IGM were felt to be pre-diabetics, but data from several epidemiological studies argue that subjects with IGM are heterogeneous with respect to their risk of diabetes and their risk of cardiovascular morbidity and mortality. The data suggest that subjects with IGM, in particular, those with impaired glucose tolerance (IGT), do not always develop diabetes, but whether they are diabetic or not, they are, nonetheless, at high risk for cardiovascular morbidity and mortality. Among subjects with IGM, about 58% have Impaired Glucose tolerance (IGT), another 29% have impaired fasting glucose (IFG), and 13% have both abnormalities (IFG/IGT). As discussed above, IGT is characterized by elevated post-prandial (post-meal) hyperglycemia while IFG has been defined by the ADA (American Diabetes Association) on the basis of fasting glycemic values.

The categories of (a) normal glucose tolerance (NGT), (b) impaired glucose metabolism (IGM) and (c) overt type 2 diabetes mellitus are periodically revised and adopted by the Expert Committee of the American Diabetes Association (ADA). The actual values as defined in “Report of the Expert Committee on the Diagnosis and Classification of Diabetes Mellitus. Diabetes Care (26) 1, 2003, 5-20” and “The Diabetes Ready-Reference Guide for Health Care Professionals, 2000, published by the American Diabetes Association” are:

a) Normal Glucose Tolerance (NGT)=fasting glucose level <6.1 mmol/L or less than 110 mg/dl and a 2 h post-prandial glucose level of <7.8 mmol/L or <140 mg/dl.

b) Impaired Glucose Metabolism (IGM) is impaired fasting glucose (IFG) defined as IFG=fasting glucose level of 6.1-7.0 mmol/L or 110-126 mg/dl and/or impaired glucose tolerance (IGT)=a 2 h post-prandial glucose level (75 g OGTT) of 7.8-11.1 mmol/L or 140-200 mg/dl).

c) Type 2 diabetes=fasting glucose of greater than 7 mmol/L or 126 mg/dl or a 2 h post-prandial glucose level (75 g OGTT) of greater than 11.1 mmolIL or 200 mg/dl.

These criteria were defined using the WHO recommended conditions for administration of an oral glucose tolerance test (75 g OGTT) i.e., the oral administration of a glucose load containing the equivalent of 75 g of anhydrous glucose dissolved in water with a blood sample taken 2 hours later to analyze to post-prandial glucose. Other OGTT test conditions have confirmed the associated risks of the IGT and IFG categories including: 1) using 50 g glucose instead of 75 g, 2) using a casual (non-fasting) glucose sample as the analyte, and 3) analysing the post-prandial glucose at 1 hour rather than 2 hours post-glucose load. Under all of these conditions, the glycemic categories defined above have been linked to the increased risks described below, but the standardized OGTT is preferred in order to minimize variations in test results.

Insulin resistance is not primarily due to a diminished number of insulin receptors but to a post-insulin receptor binding defect that is not yet understood. This resistance to insulin responsiveness results in insufficient insulin activation of glucose uptake, oxidation and storage in muscle and inadequate insulin repression of lipolysis in adipose tissue and of glucose production and secretion in the liver.

Accordingly, the compounds and combinations of the present invention are eespecially useful for the treatment of pathological states, selected from the group consisting of IGT, IFG and IGM.

### SUMMARY OF THE INVENTION

The present invention provides a compound of the formula

NR¹R²—C(═EWG1)—(CR³R⁴)ₙ—CR⁵R⁶—CR⁷R⁸-CR⁹(NR¹⁰R¹¹)—C(═EWG2)—PM (I)

wherein n is 0 or 1;

wherein R¹, R², R³, R⁴, R⁵, R⁶, R⁷, R⁸, R⁹, R¹⁰, and R¹¹, independently of each other, are

a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R²⁰), a boronic acid group (—B(OH)₂), a cyano group (—C≡N), a carboxylic acid group (—COOH), a carboxylic acid ester group (—COOR²¹), a carboxylic acid anhydride group (—CO—O—CO—R²²), a hydroxamic acid group (—CO—NH(OH)), a N-substituted hydroxamic acid group (—CO—NR²³ (OH)), a O-substituted hydroxamic acid group (—CO—NH(OR²⁴)), a carboxamide group (—CO—NH₂), a N-substituted or N,N-disubstituted carboxylic acid amide group, (—CO—NHR²⁵; —CO—NR²⁶, R²⁷), an amido group (—HN—CO—R²⁸), a sulfonic acid group (—SO₃H), a sulfonamide group (—SO₂—NH₂), a N-substituted or N,N-disubstituted sulfonamide group (—SO₂—NHR²⁹; —SO₂—NR³⁰R³¹), an amidosulfone group (—NH—SO₂—R³²), a sulfone group (—SO₂—R³³), a phosphoric acid group (—OP(═O)(OH)₂), a phosphoric acid ester group (—OP(═O)(OR³⁴)(OR³⁵)), a phosphonic acid group (—P(═O)(OH)₂), an phosphonic acid ester group (—P(═O)(OR³⁶)(OR³⁷)), a halogen atom, a trifluormethyl group (—CF₃), a thiol group (—SH); a thioether group (—S—R³⁸), a hydroxy group (—OH); an alkoxy group (—O—R³⁹), a tetrazole group, an amino group (—NH₂), or a N-substituted or N,N-disubstituted amino group (—NHR⁴⁰; —NR⁴R⁴²);

which each independently can be substituted with one or more substituents, which can be the same or different; and,

wherein optionally, any two of the groups R¹, R², R³, R⁴, R⁵, R⁶, R⁷, R⁸, R⁹, R¹⁰, and R¹, as well the pairs R²⁶/R²⁷, R³⁰/R³¹, R³⁴/R³⁵, R³⁶/R³⁷ and R⁴¹/R⁴², independenly of each other, may form a part of a ring; and

wherein the substituents R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, and R⁴² independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group; and

wherein EWG1 and EWG2 are each independently an electron withdrawing group and;

wherein the group PM

has the formula (II)

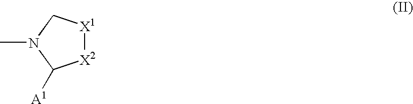

wherein X¹ is CR⁵¹R¹², O, S, SO, SO₂ or NR⁵³; and

wherein X² is CR⁵⁴R⁵⁵, O, S, SO, SO₂, or NR⁵⁶; and

wherein R⁵¹, R⁵², R⁵³, R⁵⁴, R⁵⁵, and R⁵⁵, independently of each other, are

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁶⁰), a boronic acid group (—B(OH)₂), a cyano group (—C≡N), a carboxylic acid group (—COOH), a carboxylic acid ester group (—COOR⁶), a carboxylic acid anhydride group (—CO—O—CO—R⁶²), a hydroxaniic acid group (—CO—NH(OH)), a N-substituted hydroxamic acid group (—CO—NR⁶³ (OH)), a O-substituted hydroxamnic acid group (—CO—NH(OR⁶⁴)), a carboxaiide group (—CO—NH₂), a N-substituted or N,N-disubstituted carboxylic acid amide group, (—CO—NHR⁶⁵; —CO—NR⁶⁶R⁶), an amido group (—HN—CO—R⁶⁸), a sulfonic acid group (—SO₃H), a sulfonamide group (—SO₂—NH₂), a N-substituted or N,N-disubstituted sulfonamide group (—SO₂—NHR⁶⁹; —SO₂—NR⁷⁰R⁷¹), an amidosulfone group (—NH—SO₂—R⁷²), a sulfone group (—SO₂—R⁷³), a phosphoric acid group (—OP(═O)(OH)₂), a phosphoric acid ester group (—OP(═O)(OR⁷⁴)(OR⁷⁵)), a phosphonic acid group (—P(═O)(OH)₂), an phosphonic acid ester group (—P(═O)(OR⁷⁶)(OR⁷⁷)), a halogen atom, a trifluormethyl group (—CF₃), a thiol group (—SH); a thioether group (—S—R⁷⁸), a hydroxy group (—OH); an alkoxy group (—O—R⁷⁹), a tetrazole group, an amino group (—NH₂), or a N-substituted or N,N-disubstituted amino group (—NHR⁸⁰; —NR⁸R⁸²); and

which, independently of each other, can be substituted with one or more substituents, which can be the same or different; and,

wherein optionally, any two of the groups R⁵¹, R⁵², R⁵³, R⁵⁴, R⁵⁵, and R⁵⁶, if present, as well as the pairs R⁶⁶/R⁶⁷, R⁷⁰/R⁷¹, R⁷⁴/R⁷⁵, R⁷⁶/R⁷⁷ and R⁸¹/R⁸², independently of each other, may form a part of a ring; and

wherein the substituents R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³R⁷⁴, R⁷⁵, R⁷⁶, R⁷⁷, R⁷, R⁷⁹, R⁸⁰, R⁸¹, and R⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group; and

wherein A¹ is

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R¹⁰⁰), a boronic acid group (—B(OH)₂), a cyano group (—C≡N), a carboxylic acid group (—COOH), a carboxylic acid ester group (—COOR¹⁰¹), a carboxylic acid anhydride group (—CO—O—CO—R¹⁰²), a hydroxamic acid group (—CO—NH(OH)), a N-substituted hydroxamic acid group (—CO—NR¹⁰³(OH)), a O-substituted hydroxamic acid group (—CO—NH(OR¹⁰⁴)), a carboxamide group (—CO—NH₂), a N-substituted or N,N-disubstituted carboxylic acid amide group, (—CO—NHR¹⁰⁵; —CO—NR¹⁰⁶R¹⁰⁷), an amido group (—HN—CO—R¹⁰⁸), a sulfonic acid group (—SO₃H), a sulfonamide group (—SO₂—NH₂), a N-substituted or N,N-disubstituted sulfonamide group (—SO₂—NHR¹⁰⁹; —SO₂—NR¹¹⁰R¹¹¹), an amidosulfone group (—NH—SO₂—R¹¹²), a sulfone group (—SO₂—R¹¹³), a phosphoric acid group (—OP(═O)(OH)₂), a phosphoric acid ester group (—OP(═O)(OR¹¹⁴)(OR¹¹⁵)), a phosphonic acid group (—P(═O)(OH)₂), an phosphonic acid ester group (—P(═O)(OR¹¹⁶)(OR¹¹⁷)), a halogen atom, a trifluormethyl group (—CF₃), a thiol group (—SH); a thioether group (—S—R¹¹⁸), a hydroxy group (—OH); an alkoxy group (—O—R¹¹⁹), a tetrazole group, an amino group (—NH₂), or a N-substituted or N,N-disubstituted amino group (—NHR¹²⁰; —NR¹²¹R¹²²); and

which, independently of each other, can be substituted with one or more substituents, which can be the same or different; and,

wherein optionally, the pairs R¹⁰⁶/R¹⁰⁷, R¹¹⁰/R¹¹, R¹¹⁴/R¹⁵⁵, R¹¹⁶/R¹¹⁷ and R¹²¹/R¹²², independently of each other, may form a part of a ring; and

wherein the substituents R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R^{111, R}¹¹², R¹¹³, R¹¹⁴, R¹¹⁵, R¹¹⁶, R¹¹⁷, R¹¹⁸, R¹¹⁹, R¹²⁰, R¹²¹, and R¹²², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (III)

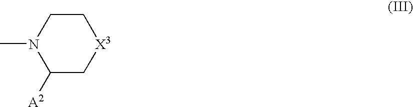

wherein X³ is CR¹³¹, R¹³², O, S, SO, SO₂, or NR¹³³; and

wherein R¹³¹, R¹³², and R¹³³, independently of each other, are

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R¹⁴⁰), a boronic acid group (—B(OH)₂), a cyano group (—C≡N), a carboxylic acid group (—COOH), a carboxylic acid ester group (—COOR¹⁴¹), a carboxylic acid anhydride group (—CO—O—CO—R¹⁴²), a hydroxamic acid group (—CO—NH(OH)), a N-substituted hydroxamic acid group (—CO—NR¹⁴³(OH)), a O-substituted hydroxamic acid group (—CO—NH(OR¹⁴⁴)), a carboxamide group (—CO—NH₂), a N-substituted or N,N-disubstituted carboxylic acid amide group, (—CO—NHR¹⁴⁵; —CO—NR¹⁴⁶R¹⁴⁷), an amido group (—HN—CO—R¹⁴⁸), a sulfonic acid group (—SO₃H), a sulfonamide group (—SO₂—NH₂), a N-substituted or N,N-disubstituted sulfonamide group (—SO₂—NHR¹⁴⁹; —SO₂—NR¹⁵⁰R¹⁵¹), an amidosulfone group (—NH—SO₂—R¹⁵²), a sulfone group (—SO₂—R¹⁵³), a phosphoric acid group (—OP(═O)(OH)₂), a phosphoric acid ester group (—OP(═O)(OR¹⁵⁴)(OR¹⁵⁵)), a phosphonic acid group (—P(═O)(OH)₂), an phosphonic acid ester group (—P(═O)(OR¹⁵⁶)(OR¹⁵⁷)), a halogen atom, a trifluormethyl group (—CF₃), a thiol group (—SH); a thioether group (—S—R¹⁵⁸), a hydroxy group (—OH); an alkoxy group (—O—R¹⁵⁹), a tetrazole group, an amino group (—NH₂), or a N-substituted or N,N-disubstituted amino group (—NHR¹⁶⁰; —NR¹⁶¹R¹⁶²); and

which, independently of each other, can be substituted with one or more substituents, which can be the same or different; and,

wherein optionally, the the pair R¹³¹/R¹³², if present, as well the pairs R¹⁴⁶/R¹⁴⁷, R¹⁵⁰/R¹⁵¹, R¹⁵⁴/R¹⁵⁵, R¹⁵⁶/R¹⁵⁷ and R¹⁶¹/R¹⁶², independenly of each other, may form a part of a ring; and

wherein the substituents R¹⁴⁰, R¹⁴¹, R¹⁴², R¹⁴³, R¹⁴⁴, R¹⁴⁵, R¹⁴⁶, R¹⁴⁷, R¹⁴⁸, R¹⁴⁹, R¹⁵⁰, R¹⁵¹, R¹⁵², R¹⁵³, R¹⁵⁴, R¹⁵⁵, R¹⁵⁶, R¹⁵⁷, R¹⁵⁸, R¹⁵⁹, R¹⁶⁰, R¹⁶¹, and R¹⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

wherein A² is

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R¹⁸⁰), a boronic acid group (—B(OH)₂), a cyano group (—C≡N), a carboxylic acid group (—COOH), a carboxylic acid ester group (—COOR¹⁸¹), a carboxylic acid anhydride group (—CO—O—CO—R⁸²), a hydroxamic acid group (—CO—NH(OH)), a N-substituted hydroxamic acid group (—CO—NR¹⁸³(OH)), a O-substituted hydroxamic acid group (—CO—NH(OR¹⁸⁴)), a carboxamide group (—CO—NH₂), a N-substituted or N,N-disubstituted carboxylic acid amide group, (—CO—NHR¹⁸⁵; —CO—NR¹⁸⁶R¹⁸⁷), an amido group (—HN—CO—R¹⁸⁸), a sulfonic acid group (—SO₃H), a sulfonamide group (—SO₂—NH₂), a N-substituted or N,N-disubstituted sulfonamide group (—SO₂—NHR¹⁸⁹; —SO₂—NR¹⁹⁰R¹⁹¹), an amidosulfone group (—NH—SO₂—R¹⁹²), a sulfone group (—SO₂—R¹⁹³), a phosphoric acid group (—OP(═O)(OH)₂), a phosphoric acid ester group (—OP(═O)(OR¹⁹⁴)(OR¹⁹⁵)), a phosphonic acid group (—P(═O)(OH)₂), an phosphonic acid ester group (—P(═O)(OR¹⁹⁶)(OR¹⁹⁷)), a halogen atom, a trifluormethyl group (—CF₃), a thiol group (—SH); a thioether group (—S—R¹⁹⁸), a hydroxy group (—OH); an alkoxy group (—O—R¹⁹⁹), a tetrazole group, an amino group (—NH₂), or a N-substituted or N,N-disubstituted amino group (—NHR²⁰⁰; —NR²⁰¹R²⁰²); and

which, independently of each other, can be substituted with one or more substituents, which can be the same or different; and,

wherein optionally, the pairs R¹⁸⁶/R¹⁸⁷, R¹⁹⁰/R¹⁹¹, R¹⁹⁴/R¹⁹⁵, R¹⁹⁶/R¹⁹⁷ and R²⁰¹/R²⁰² independenly of each other, may form a part of a ring; and

wherein the substituents R¹⁸⁰, R¹⁸¹, R¹⁸², R¹⁸³, R¹⁸⁴, R¹⁸⁵, R¹⁸⁶, R¹⁸⁷, R¹⁸⁸, R¹⁸⁹, R¹⁹⁰, R¹⁹¹, R¹⁹², R¹⁹³, R¹⁹⁴, R¹⁹⁵, R¹⁹⁶, R¹⁹⁷, R¹⁹⁸, R¹⁹⁹, R²⁰⁰, R²⁰¹, and R²⁰², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (IV)

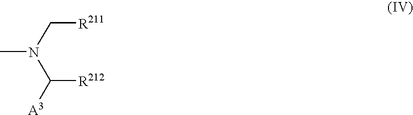

wherein R²¹¹ and R²¹², independently of each other, are

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R²²⁰), a boronic acid group (—B(OH)₂), a cyano group (—C≡N), a carboxylic acid group (—COOH), a carboxylic acid ester group (—COOR²²), a carboxylic acid anhydride group (—CO—O—CO—R²²²), a hydroxamic acid group (—CO—NH(OH)), a N-substituted hydroxamic acid group (—CO—NR²²³(OH)), a O-substituted hydroxamnic acid group (—CO—NH(OR²²⁴)), a carboxaniide group (—CO—NH₂), a N-substituted or N,N-disubstituted carboxylic acid amide group, (—CO—NHR²²⁵; —CO—NR²²⁶, R²²⁷), an amido group (—HN—CO—R²²⁸), a sulfonic acid group (—SO₃H), a sulfonamide group (—SO₂—NH₂), a N-substituted or N,N-disubstituted sulfonamide group (—SO₂—NHR²²⁹-SO₂—NR²³⁰R²³¹), an amidosulfone group (—NH—SO₂—R²³²), a sulfone group (—SO₂—R²³³), a phosphoric acid group (—OP(═O)(OH)₂), a phosphoric acid ester group (—OP(═O)(OR²³⁴)(OR²³⁵)), a phosphonic acid group (—P(═O)(OH)₂), an phosphonic acid ester group (—P(═O)(OR²³⁶)(OR²³⁷)), a halogen atom, a trifluormethyl group (—CF₃), a thiol group (—SH); a thioether group (—S—R²³⁸), a hydroxy group (—OH); an alkoxy group (—O—R²³⁹), a tetrazole group, an amino group (—NH₂), or a N-substituted or N,N-disubstituted amino group (—NHR²⁴⁰; —NR²⁴¹R²⁴²); and

which, independently of each other, can be substituted with one or more substituents, which can be the same or different; and,

wherein optionally, the pairs R²²⁶/R²²⁷, R²³⁰/R²¹³, R²³⁴/R²³⁵, R²³⁶/R²³⁷ and R²⁴¹/R²⁴² independenly of each other, may form a part of a ring; and

wherein the substituents R²²⁰, R²²¹, R²²², R²²³, R²²⁴, R²²⁵, R²²⁶, R²²⁷, R²²⁸, R²²⁹, R²³⁰ R²³¹, R²³², R²³³, R²³⁴, R²³⁵, R²³⁶, R²³⁷, R²³⁸, R²³⁹, R²⁴⁰, R²⁴¹, and R²⁴², independently of 20 each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

wherein A³ is

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R²⁶⁰), a boronic acid group (—B(OH)₂), a cyano group (—C≡N), a carboxylic acid group (—COOH), a carboxylic acid ester group (—COOR²⁶¹), a carboxylic acid anhydride group (—CO—O—CO—R²⁶²), a hydroxamic acid group (—CO—NH(OH)), a N-substituted hydroxamic acid group (—CO—NR²⁶³ (OH)), a O-substituted hydroxamic acid group (—CO—NH(OR²⁶⁴)), a carboxamide group (—CO—NH₂), a N-substituted or N,N-disubstituted carboxylic acid amide group, (—CO—NHR²⁶⁵; —CO—NR²⁶⁶R²⁶⁷), an amido group (—HN—CO—R²⁶⁸), a sulfonic acid group (—SO₃H), a sulfonamide group (—SO₂—NH₂), a N-substituted or N,N-disubstituted sulfonamide group (—SO₂—NHR²⁶⁹-SO₂—NR²¹⁰R²⁷¹), an amidosulfone group (—NH—SO₂—R²⁷²), a sulfone group (—SO₂—R²⁷³), a phosphoric acid group (—OP(═O)(OH)₂), a phosphoric acid ester group (—OP(═O)(OR²⁷⁴)(OR²⁷⁵)), a phosphonic acid group (—P(═O)(OH)₂), an phosphonic acid ester group (—P(═O)(OR²⁷⁶)(OR²⁷⁷)), a halogen atom, a trifluormethyl group (—CF₃), a thiol group (—SH); a thioether group (—S—R²⁷⁸), a hydroxy group (—OH); an alkoxy group (—O—R²⁷⁹), a tetrazole group, an amino group (—NH₂), or a N-substituted or N,N-disubstituted amino group (—NHR²⁸⁰; —NR²⁸¹R²⁸²); and

which, independently of each other, can be substituted with one or more substituents, which can be the same or different; and,

wherein optionally, the pairs R²⁶⁶/R²⁶⁷, R²⁷⁰/R²⁷¹, R²⁷⁴/R²⁷⁵, R²⁷⁶/R²⁷⁷ and R²⁸¹/R²⁸², independenly of each other, may form a part of a ring; and

wherein the substituents R²⁶⁰, R²⁶¹, R²⁶², R²⁶³, R²⁶⁴, R²⁶⁵, R²⁶⁶, R²⁶⁷, R²⁶⁸, R²⁶⁹, R²⁷⁰, R²⁷¹, R²⁷², R²⁷³, R²⁷⁴, R²⁷⁵, R²⁷⁶, R²⁷⁷, R²⁷⁸, R²⁷⁹, R²⁸⁰, R²⁸¹, and R²⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (V)

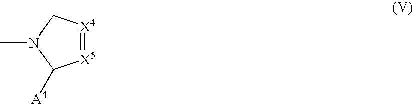

wherein X⁴ iS CR²⁹¹ or N; and

wherein X⁵ is CR²⁹² or N; and

wherein R²⁹¹ and R²⁹², independently of each other, are

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R³⁰⁰), a boronic acid group (—B(OH)₂), a cyano group (—C≡N), a carboxylic acid group (—COOH), a carboxylic acid ester group (—COOR³⁰¹), a carboxylic acid anhydride group (—CO—O—CO—R³⁰²), a hydroxamic acid group (—CO—NH(OH)), a N-substituted hydroxamic acid group (—CO—NR³⁰³ (OH)), a O-substituted hydroxamic acid group (—CO—NH(OR³⁰⁴)), a carboxamide group (—C O—NH₂), a N-substituted or N,N-di substituted carboxylic acid amide group, (—CO—NHR³⁰⁵; —CO—NR³⁰⁶R³⁰⁷), an amido group (—HN—CO—R³⁰⁸), a sulfonic acid group (—SO₃H), a sulfonamide group (—SO₂—NH₂), a N-substituted or N,N-disubstituted sulfonamide group (—SO₂—NHR³⁰⁹; —SO₂—NR³¹OR³¹¹), an amidosulfone group (—NH—SO₂—R³¹²), a sulfone group (—SO₂—R³¹³), a phosphoric acid group (—OP(═O)(OH)₂), a phosphoric acid ester group (—OP(═O)(OR³¹⁴)(OR³¹⁵)), a phosphonic acid group (—P(═O)(OH)₂), an phosphonic acid ester group (—P(═O)(OR³¹⁶)(OR³¹⁷)), a halogen atom, a trifluormethyl group (—CF₃), a thiol group (—SH); a thioether group (—S—R³¹⁸), a hydroxy group (—OH); an alkoxy group (—O—R³¹⁹), a tetrazole group, an amino group (—NH₂), or a N-substituted or N,N-disubstituted amino group (—NHR³²⁰; —NR³²¹R³²²); and

which, independently of each other, can be substituted with one or more substituents, which can be the same or different; and,

wherein optionally, the the pair R²⁹¹/R²⁹², if present, as well the pairs R³⁰⁶/R³⁰⁷, R³¹⁰/R³¹¹, R³¹⁴/R³¹⁵, R³¹⁶/R³¹⁷ and R³²¹/R³²², independenly of each other, may form a part of a ring; and

wherein the substituents R³⁰⁰, R³⁰¹, R³⁰², R³⁰³, R³⁰⁴, R³⁰⁵, R³⁰⁶, R³⁰⁷, R³⁰⁸, R³⁰⁹, R³¹⁰, R³¹¹, R³¹², R³¹³, R³¹⁴, R³¹⁵, R³¹⁶, R³¹⁷, R³¹⁸, R³¹⁹, R³²⁰, R³²¹, and R³²², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

wherein A⁴ is

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R³⁴⁰), a boronic acid group (—B(OH)₂), a cyano group (—C≡N), a carboxylic acid group (—COOH), a carboxylic acid ester group (—COOR³⁴¹), a carboxylic acid anhydride group (—CO—O—CO—R³⁴²), a hydroxamic acid group (—CO—NH(OH)), a N-substituted hydroxamic acid group (—CO—NR³⁴³(OH)), a O-substituted hydroxamic acid group (—CO—NH(OR³⁴⁴)), a carboxamide group (—CO—NH₂), a N-substituted or N,N-disubstituted carboxylic acid amide group, (—CO—NHR³⁴⁵; —CO—NR³⁴⁶R³⁴⁷), an amido group (—HN—CO—R³⁴⁸), a sulfonic acid group (—SO₃H), a sulfonamide group (—SO₂—NH₂), a N-substituted or N,N-disubstituted sulfonamide group (—SO₂—NHR³⁴⁹; —SO₂—NR³⁵⁰R³⁵¹), an amidosulfone group (—NH—SO₂—R³⁵²), a sulfone group (—SO₂—R³⁵³), a phosphoric acid group (—OP(═O)(OH)₂), a phosphoric acid ester group (—OP(═O)(OR³⁵⁴)(OR³⁵⁵)), a phosphonic acid group (—P(═O)(OH)₂), an phosphonic acid ester group (—P(═O)(OR³⁵⁶)(OR³⁵⁷)), a halogen atom, a trifluormethyl group (—CF₃), a thiol group (—SH); a thioether group (—S—R³⁵⁸), a hydroxy group (—OH); an alkoxy group (—O—R³⁵⁹), a tetrazole group, an amino group (—NH₂), or a N-substituted or N,N-disubstituted amino group (—NHR³⁶⁰; —NR³⁶¹R³⁶²); and

which, independently of each other, can be substituted with one or more substituents, which can be the same or different; and,

wherein optionally, the pairs R³⁴⁶/R³⁴⁷, R³⁵⁰/R³⁵P, R³⁵⁴/R³⁵⁵, R³⁵⁶/R³⁵⁷ and R³⁶¹/R³⁶², independenly of each other, may form a part of a ring; and

wherein the substituents R³⁴⁰, R³⁴¹, R³⁴², R³⁴³, R³⁴⁴, R³⁴⁵, R³⁴⁶, R³⁴⁷, R³⁴⁸, R³⁴⁹, R³⁵⁰, R³⁵¹R³⁵², R³⁵³, R³⁵⁴, R³⁵⁵, R³⁵⁶, R³⁵⁷, R³⁵⁸, R³⁵⁹, R³⁶⁰, R³⁶¹, and R³⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (VI)

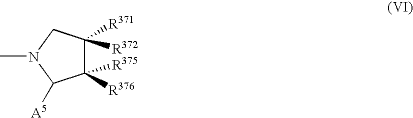

wherein R³⁷¹, R³⁷², R³⁷⁵ and R³⁷⁶, independently of each other, a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R³⁸⁰), a boronic acid group (—B(OH)₂), a cyano group (—C≡N), a carboxylic acid group (—COOH), a carboxylic acid ester group (—COOR³⁸¹), a carboxylic acid anhydride group (—CO—O—CO—R³⁸²), a hydroxamic acid group (—CO—NH(OH)), a N-substituted hydroxamic acid group (—CO—NR³⁸³ (OH)), a O-substituted hydroxamic acid group (—CO—NH(OR³⁸⁴)), a carboxamide group (—CO—NH₂), a N-substituted or N,N-disubstituted carboxylic acid amide group, (—CO—NHR³⁸⁵; —CO—NR³⁸⁶R³⁸⁷), an amido group (—HN—CO—R³⁸⁸), a sulfonic acid group (—SO₃H), a sulfonamide group (—SO₂—NH₂), a N-substituted or N,N-disubstituted sulfonamide group (—SO₂—NHR³⁸⁹; —SO₂—NR³⁹⁰R³⁹¹), an amidosulfone group (—NH—SO₂—R³⁹²), a sulfone group (—SO₂—R³⁹³), a phosphoric acid group (—OP(═O)(OH)₂), a phosphoric acid ester group (—OP(═O)(OR³⁹⁴)(OR³⁹⁵)), a phosphonic acid group (—P(═O)(OH)₂), an phosphonic acid ester group (—P(═O)(OR³⁹⁶)(OR³⁹⁷)), a halogen atom, a trifluormethyl group (—CF₃), a thiol group (—SH); a thioether group (—S—R³⁹⁸), a hydroxy group (—OH); an alkoxy group (—O—R³⁹⁹), a tetrazole group, an amino group (—NH₂), or a N-substituted or N,N-disubstituted amino group (—NHR⁴⁰⁰; —NR⁴⁰¹R⁴⁰²); and

which, independently of each other, can be substituted with one or more substituents, which can be the same or different; and,

wherein optionally, any two of the groups R³⁷¹, R³⁷², R³⁷⁵, and R³⁷⁶, as well as the pairs R³⁸⁶/R³⁸⁷, R³⁹⁰/R³⁹¹, R³⁹⁴/R³⁹⁵, R³⁹⁶/R³⁹⁷ and R⁴⁰¹/R⁴⁰², independenly of each other, may form a part of a ring; and

wherein the substituents R³⁸⁰, R³⁸¹, R³⁸², R³⁸³, R³⁸⁴, R³⁸⁵, R³⁸⁶, R³⁸⁷, R³⁸⁸, R³⁸⁹, R³⁹⁰, R³⁹¹, R³⁹², R³⁹³, R³⁹⁴, R³⁹⁵, R³⁹⁶, R³⁹⁷, R³⁹⁸, R³⁹⁹, R⁴⁰⁰, R⁴⁰¹, and R⁴⁰², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group; or

alternatively; the two groups R³⁷¹ and R³⁷² can be together an oxo (═O) or hydroxyimino (═N—OH) group; and

alternatively; the two groups R³⁷⁵ and R³⁷⁶ can be together an oxo (═O) or hydroxyimino (═N—OH) group; and

wherein A⁵ is

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁴²⁰), a boronic acid group (—B(OH)₂), a cyano group (—C≡N), a carboxylic acid group (—COOH), a carboxylic acid ester group (—COOR⁴²¹), a carboxylic acid anhydride group (—CO—O—CO—R⁴²²), a hydroxamic acid group (—CO—NH(OH)), a N-substituted hydroxamic acid group (—CO—NR⁴²³(OH)), a O-substituted hydroxamic acid group (—CO—NH(OR⁴²⁴)), a carboxamide group (—CO—NH₂), a N-substituted or N,N-disubstituted carboxylic acid amide group, (—CO—NHR⁴²⁵; —CO—NR⁴²⁶R⁴²), an amido group (—HN—CO—R⁴²⁸), a sulfonic acid group (—SO₃H), a sulfonamide group (—SO₂—NH₂), a N-substituted or N,N-disubstituted sulfonamide group (—SO₂—NHR⁴²⁹; —SO₂—NR⁴³⁰R⁴³¹), an amidosulfone group (—NH—SO₂—R⁴³²), a sulfone group (—SO₂—R⁴³³), a phosphoric acid group (—OP(═O)(OH)₂), a phosphoric acid ester group (—OP(═O)(OR⁴³⁴)(OR⁴³⁵)), a phosphonic acid group (—P(═O)(OH)₂), an phosphonic acid ester group (—P(═O)(OR⁴⁶)(OR⁴³¹)), a halogen atom, a trifluormethyl group (—CF₃), a thiol group (—SH); a thioether group (—S—R⁴³⁸), a hydroxy group (—OH); an alkoxy group (—O—R⁴³⁹), a tetrazole group, an amino group (—NH₂), or a N-substituted or N,N-disubstituted amino group (—NHR⁴⁴⁰; —NR⁴⁴R⁴⁴²); and

which, independently of each other, can be substituted with one or more substituents, which can be the same or different; and,

wherein optionally, the pairs R⁴²⁶/R⁴²⁷, R⁴³⁰/R⁴³¹, R⁴³⁴/R⁴³⁵, R⁴³⁶/R⁴³⁷ and R⁴⁴¹/R⁴⁴², independenly of each other, may form a part of a ring; and

wherein the substituents R⁴²⁰, R⁴²¹, R⁴²², R⁴²³, R⁴²⁴, R⁴²⁵, R⁴²⁶, R⁴²⁷, R⁴²⁸, R⁴²⁹, R⁴³⁰, R⁴³¹, R⁴²³, R⁴³³, R⁴³⁴, R⁴³⁵, R⁴³⁶, R⁴³⁷, R⁴³⁸, R⁴³⁹, R⁴⁴⁰, R⁴⁴¹, and R⁴⁴², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (VII)

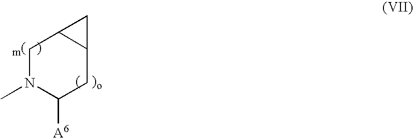

wherein m is equal to 1 or 2, and o is equal to 1 or 2, and m or o can be 0;

wherein A⁶ is a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁴⁶⁰), a boronic acid group (—B(OH)₂), a cyano group (—C≡N), a carboxylic acid group (—COOH), a carboxylic acid ester group (—COOR⁴⁶¹), a carboxylic acid anhydride group (—CO—O—CO—R⁴⁶²), a hydroxamic acid group (—CO—NH(OH)), a N-substituted hydroxamic acid group (—CO—NR⁴⁶³(OH)), a O-substituted hydroxamic acid group (—CO—NH(OR⁴⁶⁴)), a carboxamide group (—CO—NH₂), a N-substituted or N,N-disubstituted carboxylic acid amide group, (—CO—NHR⁴⁶⁵; —CO—NR⁴⁶⁶R⁴⁶⁷), an amido group (—HN—CO—R⁴⁶⁸), a sulfonic acid group (—SO₃H), a sulfonamide group (—SO₂—NH₂), a N-substituted or N,N-disubstituted sulfonamide group (—SO₂—NHR⁴⁶⁹; —SO₂—NR⁴⁷⁰R⁴⁷¹), an amidosulfone group (—NH—SO₂—R⁴⁷²), a sulfone group (—SO₂—R⁴⁷³), a phosphoric acid group (—OP(═O)(OH)₂), a phosphoric acid ester group (—OP(═O)(OR⁴⁷⁴)(OR⁴⁷⁵)), a phosphonic acid group (—P(═O)(OH)₂), an phosphonic acid ester group (—P(═O)(OR⁴⁷⁶)(OR⁴⁷⁷)), a halogen atom, a trifluormethyl group (—CF₃), a thiol group (—SH); a thioether group (—S—R⁴⁷⁸), a hydroxy group (—OH); an alkoxy group (—O—R⁴⁷⁹), a tetrazole group, an amino group (—NH₂), or a N-substituted or N,N-disubstituted amino group (—NHR⁴⁸⁰; —NR⁴⁸¹R⁴⁸²);

which, independently of each other, can be substituted with one or more substituents, which can be the same or different; and,

wherein optionally, the pairs R⁴⁶⁶/R⁴⁶⁷, R⁴⁷⁰/R⁴⁷¹, R⁴⁷⁴/R⁴⁷⁵, R⁴⁷⁶/R⁴⁷⁷ and R⁴⁸¹/R⁴⁸², independenly of each other, may form a part of a ring; and

wherein the substituents R⁴⁶⁰, R⁴⁶¹, R⁴⁶², R⁴⁶³, R⁴⁶⁴, R⁴⁶⁵, R⁴⁶⁶, R⁴⁶⁷, R⁴⁶⁸, R⁴⁶⁹, R⁴⁷⁰, R⁴⁷¹, R⁴⁷², R⁴⁷³, R⁴⁷⁴, R⁴⁷⁵, R⁴⁷⁶, R⁴⁷⁷, R⁴⁷⁸, R⁴⁷⁹, R⁴⁸⁰, R⁴⁸¹, and R⁴⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (VIII)

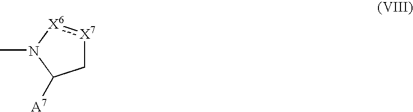

wherein X⁶ is selected from CR⁴⁹⁰R⁴⁹¹, O, S or NR⁴⁹², when the bond between X⁶ and X⁷ is a single bond; and

wherein X⁷ is selected from CR⁴⁹³R⁴⁹⁴, O, S, or NR⁴⁹⁵, when the bond between x and X is a single bond;

or alternatively,

wherein X⁶ is selected from CR⁴⁹⁶ or N, when the bond between X⁶ and X⁷ is a double bond; and

wherein X⁷ is selected from CR⁴⁹⁷ or N, when the bond between X⁶ and X⁷ is a double bond; and

wherein R⁴⁹⁰, R⁴⁹¹, R⁴⁹², R⁴⁹³, R⁴⁹⁴, R⁴⁹⁵, R⁴⁹⁶, and R⁴⁹⁷, independently of each other, are a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁵⁰⁰), a boronic acid group (—B(OH)₂), a cyano group (—C≡N), a carboxylic acid group (—COOH), a carboxylic acid ester group (—COOR⁵⁰¹), a carboxylic acid anhydride group (—CO—O—CO—R⁵⁰²), a hydroxaniic acid group (—CO—NH(OH)), a N-substituted hydroxamic acid group (—CO—NR⁵⁰³(OH)), a O-substituted hydroxamic acid group (—CO—NH(OR⁵⁰⁴)), a carboxamide group (—CO—NH₂), a N-substituted or N,N-disubstituted carboxylic acid amide group, (—CO—NHR⁵⁰⁵; —CO—NR⁵⁰⁶R⁵⁰⁷), an amido group (—HN—CO—R⁵⁰⁸), a sulfonic acid group (—SO₃H), a sulfonamide group (—SO₂—NH₂), a N-substituted or N,N-disubstituted sulfonamide group (—SO₂—NHR⁵⁰⁹; —SO₂—NR⁵¹⁰R⁵¹¹), an amidosulfone group (—NH—SO₂—R⁵¹²), a sulfone group (—SO₂—R⁵¹³), a phosphoric acid group (—OP(═O)(OH)₂), a phosphoric acid ester group (—OP(═O)(OR⁵¹⁴)(OR⁵¹⁵)) a phosphonic acid group (—P(═O)(OH)₂), an phosphonic acid ester group (—P(═O)(OR⁵¹⁶)(OR⁵¹⁷)), a halogen atom, a trifluormethyl group (—CF₃), a thiol group (—SH); a thioether group (—S—R⁵¹⁸), a hydroxy group (—OH); an alkoxy group (—O—R⁵¹⁹), a tetrazole group, an amino group (—NH₂), or a N-substituted or N,N-disubstituted amino group (—NHR⁵²⁰; —NR⁵²¹R⁵²²); and

which, independently of each other, can be substituted with one or more substituents, which can be the same or different; and,

wherein optionally, any two the groups R⁴⁰⁹, R⁴⁹¹, R⁴⁹², R⁴⁹³, R⁴⁹⁴, R⁴⁹⁵, R⁴⁹⁶, and R⁴⁹⁷, if present, as well as the pairs R⁵⁰⁶/R⁵⁰⁷, R⁵¹⁰/R⁵¹¹, R⁵¹⁴/R⁵¹⁵, R⁵¹⁶/R⁵¹⁷ and R⁵²¹/R⁵²², independenly of each other, may form a part of a ring; and

wherein the substituents R⁵⁰⁰, R⁵⁰¹, R⁵⁰², R⁵⁰³, R⁵⁰⁴, R⁵⁰⁵, R⁵⁰⁶, R⁵⁰⁷, R⁵⁰⁸, R⁵⁰⁹, R⁵¹⁰, R⁵¹¹R⁵¹², R⁵¹³, R⁵¹⁴, R⁵¹⁵, R⁵¹⁶, R⁵¹⁷, R⁵¹⁸, R⁵¹⁹, R⁵²⁰, R⁵²¹, and R²²², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group; and

wherein A⁷ is

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁵⁴⁰), a boronic acid group (—B(OH)₂), a cyano group (—C≡N), a carboxylic acid group (—COOH), a carboxylic acid ester group (—COOR⁵⁴¹), a carboxylic acid anhydride group (—CO—O—CO—R⁵⁴²), a hydroxamic acid group (—CO—NH(OH)), a N-substituted hydroxamic acid group (—CO—NR⁵⁴³(OH)), a O-substituted hydroxamic acid group (—CO—NH(OR⁵⁴⁴)), a carboxamide group (—CO—NH₂), a N-substituted or N,N-disubstituted carboxylic acid amide group, (—CO—NHR⁵⁴⁵; —CO—NR⁵⁴⁶R⁵⁴⁷), an amido group (—HN—CO—R⁵⁴⁸), a sulfonic acid group (—SO₃H), a sulfonamide group (—SO₂—NH₂), a N-substituted or N,N-disubstituted sulfonamide group (—SO₂—NHR⁵⁴⁹; —SO₂—NR⁵⁵OR⁵⁵¹), an amidosulfone group (—NH—SO₂—R⁵⁵²), a sulfone group (—SO₂—R⁵⁵³), a phosphoric acid group (—OP(═O)(OH)₂), a phosphoric acid ester group (—OP(═O)(OR⁵⁵⁴)(OR⁵⁵⁵)), a phosphonic acid group (—P(═O)(OH)₂), an phosphonic acid ester group (—P(═O)(OR⁵⁵⁶)(OR⁵⁵⁷)), a halogen atom, a trifluormethyl group (—CF₃), a thiol group (—SH); a thioether group (—S—R⁵⁵⁸), a hydroxy group (—OH); an alkoxy group (—O—R⁵⁵⁹), a tetrazole group, an amino group (—NH₂), or a N-substituted or N,N-disubstituted amino group (—NHR⁵⁶⁰; —NR⁵⁶R⁵⁶²); and

which, independently of each other, can be substituted with one or more substituents, which can be the same or different; and,

wherein optionally, the pairs R⁵⁴⁶R⁵⁴⁷, R⁵⁵⁰/R⁵⁵¹, R⁵⁵⁴/R⁵⁵⁵, R⁵⁵⁶/R⁵⁵⁷ and R⁵⁶¹/R⁵⁶², independenly of each other, may form a part of a ring; and

wherein the substituents R⁵⁴⁰, R⁵⁴¹, R⁵⁴², R⁵⁴³, R⁵⁴⁴, R⁵⁴⁵, R⁵⁴⁶, R⁵⁴⁷, R⁵⁴⁸, R⁵⁴⁹, R⁵⁵⁰, R⁵⁵¹, R⁵⁵², R⁵⁵³, R⁵⁵⁴, R⁵⁵⁵, R⁵⁵⁶, R⁵⁵⁷, R⁵⁵⁸, R⁵⁵⁹, R⁵⁶⁰, R⁵⁶¹, and R⁵⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (IX) or (IXa)

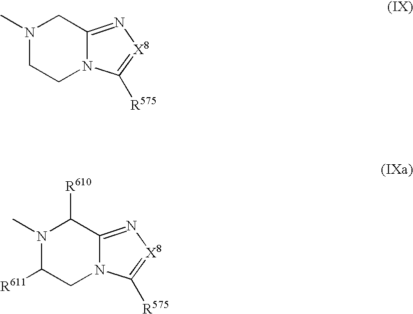

wherein X⁸ is N or CR⁵⁷⁰; and

wherein R⁵⁷⁰, R⁵⁷, R⁶¹⁰ and R⁶¹¹ independently of each other, are a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁵⁸⁰), a boronic acid group (—B(OH)₂), a cyano group (—C≡N), a carboxylic acid group (—COOH), a carboxylic acid ester group (—COOR⁵⁸¹), a carboxylic acid anhydride group (—CO—O—CO—R⁵⁸²), a hydroxamic acid group (—CO—NH(OH)), a N-substituted hydroxamic acid group (—CO—NR⁵⁸³(OH)), a O-substituted hydroxamic acid group (—CO—NH(OR⁵⁸⁴)), a carboxamide group (—CO—NH₂), a N-substituted or N,N-disubstituted carboxylic acid amide group, (—CO—NHR⁵⁸⁵; —CO—NR⁵⁸⁶, R⁵⁸⁷), an amido group (—HN—CO—R⁵⁸⁸), a suffonic acid group (—SO₃H), a sulfonamide group (—SO₂—NH₂), a N-substituted or N,N-disubstituted sulfonamide group (—SO₂—NHR⁵⁸⁹; —SO₂—NR⁵⁹⁰R⁵⁹¹), an amidosulfone group (—NH—SO₂—R⁵⁹²), a sulfone group (—SO₂—R⁵⁹³), a phosphoric acid group (—OP(═O)(OH)₂), a phosphoric acid ester group (—OP(═O)(OR⁵⁹⁴)(OR⁵⁹⁵)), a phosphonic acid group (—P(═O)(OH)₂), an phosphonic acid ester group (—P(═O)(OR⁵⁹⁶)(OR⁵⁹⁷)), a halogen atom, a trifluormethyl group (—CF₃), a thiol group (—SH); a thioether group (—S—R⁵⁹⁸), a hydroxy group (—OH); an alkoxy group (—O—R⁵⁹⁹), a tetrazole group, an amino group (—NH₂), or a N-substituted or N,N-disubstituted amino group (—NHR⁶⁰⁰; —NR⁶⁰¹R⁶⁰²);

which, independently of each other, can be substituted with one or more substituents, which can be the same or different; and,

wherein optionally, the pairs R⁵⁷⁰/R⁵⁷⁵, if present, as well as the pairs R⁵⁸⁶/R⁵⁸⁷, R⁵⁹⁰/R⁵⁹¹, R⁵⁹⁴/R⁵⁹⁵, R⁵⁹⁶/R⁵⁹⁷ and R⁶⁰¹/R⁶⁰², independenly of each other, may form a part of a ring; and

wherein the substituents R⁵⁸⁰, R⁵⁸¹, R⁵⁸², R⁵⁸³, R⁵⁸⁴, R⁵⁸⁵, R⁵⁸⁶, R⁵⁸⁷, R⁵⁸⁸, R⁵⁸⁹, R⁵⁹⁰, R⁵⁹¹, R⁵⁹², R⁵⁹³, R⁵⁹⁴, R⁵⁹⁵, R⁵⁹⁶, R⁵⁹⁷, R⁵⁹⁸, R⁵⁹⁹, R⁶⁰⁰, R⁶⁰¹, and R⁶⁰², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (X)

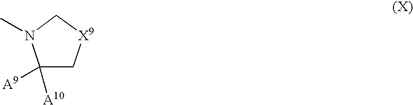

wherein the groups X⁹ is CR⁹⁰⁰R⁹⁰¹, S, SO, SO_{2 or NR}⁹⁰²

wherein R⁹⁰⁰, R⁹⁰¹ and R⁹⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR⁹¹⁰R⁹¹¹.

wherein A⁹ and A¹⁰ are, independently of each other, selected from hydrogen, cyano, —C(═O)NR⁹¹²R⁹¹³, or C₁, C₂, C₃, C₄, C₅or C₆alkyl, which is linear or branched and is optionally substituted with 1, 2, 3, 4, or 5 halogens;

wherein

R⁹¹⁰ and R⁹¹², are, independently of each other, selected from hydrogen, or C₁, C₂, C₃, C₄, C₅or C₆alkyl, which is linear or branched and is optionally substituted with 1, 2, 3, 4, or 5 halogens; and

R⁹¹¹ and R⁹¹³, are, independently of each other, selected from the group consisting of

(1) phenyl, which is optionally substituted with 1, 2, 3, 4, or 5, substituents independently selected from halogen and R⁹²⁰;

(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of

(a) hydroxy,

(b) —COOH,

(c) —COO(C₁, C₂, C₃, C₄, C₅or C₆alkyl), i.e. ester,

(d) phenyl,

(e) naphthyl,

(f) C₃, C₄, C₅or C₆cycloalkyl,

(g) a 5- or 6 membered heterocycle which may be saturated or unsaturated comprising 1, 2, 3, or 4 heteroatoms independently selected from nitrogen, oxygen or sulfur;

(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;

wherein said C₃, C₄, C₅or C₆cycloalkyl, phenyl, naphthyl, are optionally substituted with 1, 2, 3, 4, or 5 substituents independently selected from halogen and R⁹²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R⁹²⁰; and

(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OCs or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;

wherein R⁹²⁰ is selected from the group consisting of:

(1) hydroxy;

(2) cyano;

(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;

(4) C₁, C₂, C₃, C₄, C₅, C₆, C₇, C₈, Cg or C₁₀alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from

(a) hydroxy;

(b) —COOH;

(c) —COO(C₁, C₂, C₃, C₄, C₅or C₆alkyl) i.e. ester, which may linear or branched and is optionally substituted with 1, 2, 3, 4, or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR⁹²⁵R⁹²⁵;

(g) —SO₂NR⁹²⁵R⁹²⁵;

(h) —NR⁹²⁵—C(═O)R⁹²⁵

(i) —NR⁹²⁵—C(═O)NR⁹²⁵R⁹²⁵;

(j) —NR⁹²⁵ COOR⁹³⁰

(k) —O—CO—R⁹³⁰

(l) —O—CO—NR⁹²⁵R⁹²⁵;

(m) —R⁹²⁵SO₂R⁹³⁰;

(n) NR⁹²⁵R⁹²⁵;

(o) phenyl which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(5) OC₁, OC₂, 6C₃, oC₄, oC₅, oC₆, oC₇, OC₈, OC₉or OC₁₀alkyl, which is linear or branched and is optionally substituted with 0, 1, 2, 3, 4, or 5 halogen atoms and 0, 1, or 2 substitutents selected from

(a) hydroxy;

(b) —COOH;

(c) —COO(C₁, C₂, C₃, C₄, C₅or C₆alkyl) i.e. ester, which may be linear or branched and is optionally substituted with 1, 2, 3, 4 or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR⁹²⁵R⁹²⁵;

(g) —SO₂NR⁹²⁵R⁹²⁵;

(h) —NR⁹²⁵—C(═O)R⁹²⁵

(i) —NR⁹²⁵—C(═O)NR⁹²⁵R⁹²⁵;

(k) —O—CO—R⁹³⁰

(l) —O—CO—NR⁹²⁵, R⁹²⁵;

(m) NR⁹²⁵SO₂R⁹³⁰;

(n) —NR⁹²⁵R⁹²⁵;

(o) phenyl, which is optionally substituted with 1, 2, 3, 4, or 5 groups independently selected from halogen, hydroxy, C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(6) —COOH;

(7) —COO(C₁, C₂, C₃, C₄, C₅or C₆alkyl) i.e. ester, which may be linear or branched and is optionally substituted with 1, 2, 3, 4, 5 halogens;

(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1,2,3,4, or 5 halogens.

(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(10)—CONR⁹²⁵R⁹²⁵;

(11) —SO₂NR⁹²⁵R⁹²⁵;

(12) —NR⁹²⁵—C(═O)R⁹²⁵ (13) NR⁹²⁵C(═O)NR⁹²⁵R⁹²⁵;

(14) —NRC₉₂₅COOR⁹³

(15)—O—CO—R⁹³⁰

(16)—O—CO—NR⁹²⁵R⁹²⁵;

(17) —NR⁹²⁵SO₂R⁹³⁰;

(18) —NR⁹²⁵R⁹²⁵;

(19) phenyl, which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;

wherein R⁹³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.

wherein R⁹²⁵ is selected from R⁹³⁰ and hydrogen.

wherein the group PM

has the formula (XI)

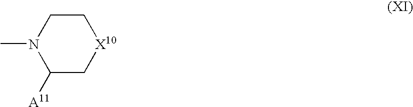

wherein the groups X¹⁰ is CR¹⁰⁰⁰R¹⁰⁰¹, S, SO, SO₂ or NR¹⁰⁰²

wherein R¹⁰⁰⁰, R¹⁰⁰¹ and R¹⁰⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR⁹¹⁰, R⁹¹¹.

and A¹¹ is selected from

hydrogen, cyano, —C(═O)NR¹⁰¹²R¹⁰¹³, or C₁, C₂, C₃, C₄, C₅or C₆ alkyl, which is linear or branched and is optionally substituted with 1, 2, 3, 4, or 5 halogens;

wherein

R¹⁰¹⁰ and R¹⁰¹², are, independently of each other, selected from hydrogen, or C₁, C₂, C₃, C₄, C₅or C₆alkyl, which is linear or branched and is optionally substituted with 1, 2, 3, 4, or 5 halogens; and

R¹⁰¹¹ and R¹⁰¹³, are, independently of each other, selected from the group consisting of

(1) phenyl, which is optionally substituted with 1, 2, 3, 4, or 5, substituents independently selected from halogen and R¹⁰²⁰;

(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of

(a) hydroxy,

(b) —COOH,

(c) —COO(C₁, C₂, C₃, C₄, C₅or C₆alkyl), i.e. ester,

(d) phenyl,

(e) naphthyl,

(f) C₃, C₄, C₅or C₆cycloalkyl,

(g) a 5- or 6 membered htereocycle which may be saturated or unsaturated comprising 1, 2, 3, or 4 heteroatoms independently selected from nitrogen, oxygen or sulfur;

(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;

wherein said C₃, C₄, C₅or C₆cycloalkyl, phenyl, naphthyl, are optionally substituted with 1, 2, 3, 4, or 5 substituents independently selected from halogen and R¹⁰²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R¹⁰²⁰; and

(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;

wherein R¹⁰²⁰ is selected from the group consisting of:

(1) hydroxy;

(2) cyano;

(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;

(4) C₁, C₂, C₃, C₄, C₅, C₆, C₇, C₈, C₉ or C₁₀ alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from

(a) hydroxy;

(b) —COOH;

(c) —COO(C₁, C₂, C₃, C₄, C₅or C₆alkyl) i.e. ester, which may linear or branched and is optionally substituted with 1, 2, 3, 4, or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC I, —OC₂, —OC₃, —OC₄, —OC₅or —OC₆alkyl being linear or branched and optionally substituted with 1, 2, 3, 4, or 5 halogens;

(f) —CONR¹⁰²⁵R¹⁰²⁵;

(g) —SO₂NR¹⁰²⁵R¹⁰²⁵;

(h) —NR¹⁰²⁵—C(═O)R¹⁰²⁵

(i) —NR¹⁰²⁵—C(═O)NR¹⁰²⁵R¹⁰²⁵;

(j) —NR¹⁰²⁵COOR¹⁰³⁰

(k) —O—CO—R¹⁰³⁰

(l) —O—CO—NR¹⁰²⁵R²⁵;

(m) —NR¹⁰²⁵ SR¹⁰³⁰;

(n) —NR¹⁰²⁵R¹⁰²⁵;

(O) phenyl which is optionally substituted with 1, 2, 3, 4, or 5 group independently selected from halogen, hydroxy, C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅ or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(5) OC₁, OC₂, OC₃, OC₄, OC₅, OC₆, OC₇, OC₈, OC₉or OC₁₀alkyl, which is linear or branched and is optionally substituted with 0, 1, 2, 3, 4, or 5 halogen atoms and 0, 1, or 2 substitutents selected from

(a) hydroxy;

(b) —COOH;

(c) —COO(C₁, C₂, C₃, C₄, C₅or C₆alkyl) i.e. ester, which may be linear or branched and is optionally substituted with 1, 2, 3, 4 or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, Cs or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR¹⁰²⁵, R¹⁰²⁵;

(g) —SO₂NR¹⁰²⁵R¹⁰²⁵;

(h) —NR¹⁰²⁵—C(═O)R¹⁰²⁵

(i) —NR¹⁰²⁵—C(═O)NR¹⁰²⁵, R¹⁰²⁵

(j) —NR¹⁰²⁵COOR¹⁰³⁰

(k) —O—CO—R¹⁰³⁰

(l) —O—CO—NR¹⁰²⁵R¹⁰²⁵;

(m) —NR¹⁰²⁵SO₂R¹⁰³⁰;

(n) _NR¹⁰²⁵R¹⁰²⁵;

(o) phenyl, which is optionally substituted with 1, 2, 3, 4, or 5 groups independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(6) —COOH;

(7) —COO(C₁, C₂, C₃, C₄, C₅or C₆alkyl) i.e. ester, which may be linear or branched and is optionally substituted with 1, 2, 3, 4, 5 halogens;

(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.

(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(10) —CONR¹⁰²⁵R¹⁰²⁵;

(11) —SO₂NR¹⁰²⁵R¹⁰²⁵;

(12) —NR¹⁰²⁵—C(═O)R¹⁰²⁵

(13)—NR¹⁰²⁵—C(═O)NR¹⁰²⁵R¹⁰²⁵

(14)—NR⁹²⁵COOR¹⁰³⁰

(15)—O—CO—R¹⁰³⁰

(16)—O—CO—NR¹⁰²⁵, R¹²⁵;

(17) —NR¹⁰²⁵ SO₂R¹⁰³⁰;

(18)—NR¹⁰²⁵R¹⁰²⁵;

(19) phenyl, which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;

wherein R¹⁰³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.

wherein R¹⁰²⁵ is selected from R¹⁰³⁰ and hydrogen.

or wherein the group PM

has the formula (XII)

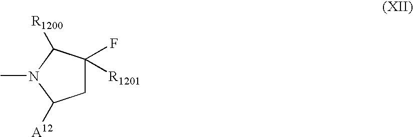

wherein the groups R¹²⁰¹ is hydrogen or fluoro.

wherein R¹²⁰⁰ und A¹² is selected from hydrogen and cyano, and the other is hydrogen.

or wherein the group PM

has the formula XIII:

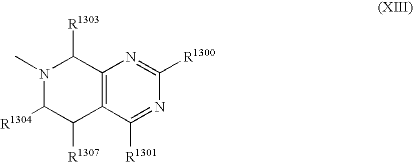

wherein:

R¹³⁰⁰ and R¹³⁰¹ are independently selected from the group consisting of:

(1) hydrogen,

(2) CN,

(3) C₁₋₁₀alkyl, which is linear or branched which is unsubstituted or substituted with:

a) halogen, or

b) phenyl, which is unsubstituted or substituted with 1-5 substitutents independently selected from halogen, CN, OH, R¹³⁰², OR¹³⁰², NHSO₂R¹³⁰² N(C₁₋₆alkyl)SO₂R¹³⁰², SO₂R¹³⁰², SO₂NR¹³⁰⁵R¹³⁰⁶, NR¹³⁰⁵R¹³⁰⁶, CONR¹³⁰⁵R¹³⁰⁶, CO₂H, and CO₂C₁₋₆alkyl, wherein the C₁₋₆alkyl is linear or branched,

(4) phenyl which is unsubstituted or substituted with 1-5 substitutents independently selected from halogen, CN, OH, R¹³⁰², OR¹³⁰², NHSO₂R¹³⁰², N(C₁₋₆alkyl)SO₂R¹³⁰², SO₂R¹³⁰², SO₂NR¹³⁰⁵R¹³⁰⁶, NR¹³⁰⁵R¹³⁰⁶, CONR¹³⁰⁵R¹³⁰⁶, CO₂H, and CO₂C₁₋₆alky wherein the C₁₋₆alkyl is linear or branched,

(5) a 5- or 6-membered heterocyclic which may be saturated or unsaturated comprising 1-4 heteroatoms independently selected from N, S and O, the heterocycle being unsubstituted or substituted with 1-3 substituents independently selected from oxo, halogen, NO₂, CN, OH, R¹³⁰², OR¹³⁰², NHSO₂R¹³⁰², N(C₁₋₆alkyl)SO₂R¹³⁰², SO₂R¹³⁰², SO₂NR¹³⁰⁵R¹³⁰⁶ NR¹³⁰⁵R¹³⁰⁶, CONR¹³⁰⁵R¹³⁰⁶, CO₂H, and CO₂C₁₋₆alkyl, wherein the C₁₋₆alkyl is linear or branched,

(6) C₃₋₆cycloalkyl, which is optionally substituted with 1-5 substituents independently selected from halogen, OH, C₁₋₆alkyl, and OC₁₋₆alkyl, wherein the C₁₋₆alkyl and OC₁₋₆ alkyl are linear or branched and optionally substituted with 1-5 halogens,

(7) OH,

(8) OR¹³⁰², and

(9) NR¹³⁰⁵R¹³⁰⁶;

R¹³⁰² is C₁₋₆alkyl, which is linear or branched and which is unsubstituted or substituted with 1-5 groups independently selected from halogen, CO₂H, and CO₂C₁₋₆alkyl, wherein the C₁₋₆alkyl is linear or branched;

R¹³⁰³, R¹³⁰⁴ and R¹³⁰⁷ are independently selected from the group consisting of:

(1) hydrogen,

(2) C₁₋₁₀alkyl, which is linear or branched and which is unsubstituted or substituted with one or more substituted selected from:

a) halogen,

b) hydroxy,

c) phenyl, which is unsubstituted or substituted with 1-5 substituted independently selected from halogen, OH, C₁₋₆alkyl, and OC₁₋₆alkyl, wherein the C₁₋₆alkyl is linear or branched and optionally substituted with 1-5 halogens,

d) naphthyl, wherein the naphthyl is optionally substituted with 1-5 substituents independently selected from halogen, OH, C₁₋₆alkyl, and OC₁₋₆alkyl, wherein the C₁₋₆alkyl is linear or branched and optionally substituted with 1-5 halogens,

e) CO₂H,

f) CO₂C₁₋₆alkyl,

g) coNR¹³⁰⁵R¹³⁰⁶;

(3) CN,

(4) phenyl which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens,

(5) naphthyl which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens,

(6) CO₂H,

(7) CO₂C₁₋₆alkyl,

(8) CONR¹³⁰⁵R¹³⁰⁶, and

(9) C₃₋₆cycloalkyl, which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens;

R¹³⁰⁵ and R¹³⁰⁶ are independently selelcted from the group consisting of:

(1) hydrogen,

(2) phenyl, which is unsubstituted or substituted with substituents independently selected from halogen, OH, C₁₋₆alkyl, and OC₁₋₆alkyl, wherein the C₁₋₆alkyl is linear or branched and optionally substituted with 1-5 halogens,

(3) C₃₋₆cycloalkyl, which is unsubstituted or substituted with 1-5 substituents independently selected from C₁₋₆alkyl, and OC₁₋₆alkyl, wherein the C₁₋₆alkyl is linear or branched and optionally substituted with 1-5 halogens

(4) C₁₋₆alkyl, which is linear or branched and which is unsubstituted or substituted with:

a) halogen, or

b) phenyl, which is unsubstituted or substituted with 1-5 substituents independently selected from halogen, OH, C₁₋₆alkyl, and OC₁₋₆alkyl, wherein the C₁₋₆alkyl is linear or branched and optionally substituted with 1-5 halogens,

or wherein R¹³⁰⁵ and R¹³⁰⁶ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆alkyl, and C₁₋₆alkoxy, wherein alkyl and alkoxy are unsubstituted with one to five halogens;

or wherein the group PM

has the formula XIV:

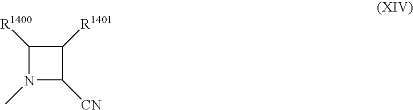

wherein R¹⁴⁰⁰ and R¹⁴⁰¹, independently of each other, are

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R¹⁴⁰²), a boronic acid group (—B(OH)₂), a cyano group (—C≡N), a carboxylic acid group (—COOH), a carboxylic acid ester group (—COOR¹⁴⁰³), a carboxylic acid anhydride group (—CO—O—CO—R¹⁴⁰⁴), a hydroxamic acid group (—CO—NH(OH)), a N-substituted hydroxamic acid group (—CO—NR¹⁴⁰⁵ (OH)), a O-substituted hydroxamic acid group (—CO—NH(OR¹⁴⁰)), a carboxamide group (—CO—NH₂), a N-substituted or N,N-disubstituted carboxylic acid amide group, (—CO—NHR¹⁴⁰⁷; —CO—NR¹⁴⁰⁸R¹⁴⁰⁹), an amido group (—HN—CO—R¹⁰), a sulfonic acid group (—SO₃H), a sulfonamide group (—SO₂—NH₂), a N-substituted or N,N-disubstituted sulfonamide group (—SO₂—NHR¹⁴¹¹; —SO₂—NR¹⁴¹²R¹⁴¹³), an amidosulfone group (—NH—SO₂—R¹⁴¹⁴), a sulfone group (—SO₂—R¹⁴¹⁵), a phosphoric acid group (—OP(═O)(OH)₂), a phosphoric acid ester group (—OP(═O)(OR¹⁴¹⁶)(OR¹⁴¹⁷)), a phosphonic acid group (—P(═O)(OH)₂), an phosphonic acid ester group (—P(═O)(OR¹⁴¹⁸)(OR¹⁴¹⁹)), a halogen atom, a trifluormethyl group (—CF₃), a thiol group (—SH); a thioether group (—S—R¹⁴²⁰), a hydroxy group (—OH); an alkoxy group (—O—R¹⁴²¹), a tetrazole group, an amino group (—NH₂), or a N-substituted or N,N-disubstituted amino group (—NHR¹⁴²²; —NR¹⁴²³R¹⁴²⁴); and

which, independently of each other, can be substituted with one or more substituents, which can be the same or different; and,

wherein optionally, the pairs R¹⁴⁰⁸/R¹⁴⁰³, R¹⁴¹²/R¹⁴¹³, R¹⁴¹⁶/R¹⁴¹⁷, R¹⁴¹⁸/R¹⁴¹⁹ and R¹⁴²³/R¹⁴²⁴, independenly of each other, may form a part of a ring; and

wherein the substituents R¹⁴⁰², R¹⁴⁰³, R¹⁴⁰⁴, R¹⁴⁰⁵, R¹⁴⁰⁶, R¹⁴⁰⁷, R¹⁴⁰⁸, R¹⁴⁰⁹, R¹⁴¹⁰, R¹⁴¹¹ R^{1412, R}¹⁴¹³, R¹⁴¹⁴, R¹⁴¹⁵, R¹⁴¹⁶, R¹⁴¹⁷, R¹⁴¹⁸, R¹⁴¹⁹, R¹⁴²⁰, R¹⁴²¹, R¹⁴²², R¹⁴²³, and R¹⁴²⁴, independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula XV:

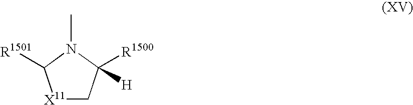

wherein X¹¹ is CH₂, CHF or CF²;

wherein R¹⁵⁰⁰ is selected from the group consisting of alkylcarbonyl, arylcarbonyl, cyano, heterocyclecarbonyl, R¹⁵⁰²R¹⁵⁰³NC(O)—, B(OR¹⁵⁰⁴)₂, (1,2,3)-dioxoborolane and 4,4,5,5-tetramethyl(1,2,3)-dioxoborolane;

wherein R¹⁵⁰¹ is selected from the group consisting of alkoxyalkyl, alkyl, alkylcarbonyl, alkenyl, alkynyl, allenyl, arylalkyl, cycloalkyl, cycloalkylalkyl, cyano, haloalkyl, haloalkenyl, heterocyclealkyl, and hydroxyalkyl;

wherein R¹⁵⁰², R¹⁵⁰³ and R¹⁵⁰⁴ are each independently selected from the group consisting of hydrogen, alkyl, and arylalkyl;

with the proviso that the following compounds are excluded:

glutaminyl thiazolidine (=Gln-Thia), glutaminyl pyrrolidine (=Gln-Pyrr) (from WO 03/072556), glutamin-pyrrolidin-2-carboxylic acid (=Gln-Pro), glutamin-pyrrolidin-2-carboxamid (=Gln-Pro amid), and (S,S) 4-Amino-5-(2-cyano-2,5-dihydro-pyrrol-1-yl)-6-oxo-pentanoic acid amide (Gln-2-cyano-2,5-dihydro-pyrrolidin) (from WO 01/55105).

### OBJECT OF THE PRESENT INVENTION

It is an object of the present invention to provide DP IV inhibitor molecules with improved bioavailability resulting in a higher transport rate from intestine into blood circulation, compared with ordinary DP IV inhibitors.

A further object of the present invention is to provide inhibitor molecules for DP IV and DP IV like enzymes, which exhibit a decreased profile of side effects in comparison with ordinary DP IV inhibitors.

Furthermore, it is an object of the present invention to provide inhibitor molecules for DP IV and DP IV like enzymes with a definite half life period in the organism, wherein the half life period can be definitely controlled by administration of a further substance in combination with DP IV inhibitors. Alternatively, the problem can be understood as an additional option which allows to control, to shorten or to prolongate the time period, during which the DP IV inhibitor is acting as an active molecule.

It is an object of the present invention to provide new DP IV inhibitors, and optionally to provide DP IV inhibitors in combination with QC inhibitors, for the manufacture of a medicament for the treatment of diseases of mammals that can be treated by modulation of DPIV- and optionally QC activity in said mammal, especially for the treatment of metabolic diseases in humans. In detail, it is the object of this invention to provide new compounds for the preparation of a medicament for the treatment of non-insulin dependent diabetes mellitus (type 2), impaired glucose tolerance, glucosuria, and disturbances of signal action at the cells of the islets of Langerhans and insulin sensitivity in the peripheral tissue in the postprandial phase of mammals, especially in humans.

Further, it is the object of this invention to provide new compounds for the preparation of a medicament for the treatment of hyperlipidemia, metabolic acidosis, diabetic neuropathy and nephropathy and of sequelae caused by diabetes mellitus in mammals; metabolism-related hypertension and cardiovascular sequelae caused by hypertension in mammals; for the prohylaxis or treatment of skin diseases and diseases of the mucosae, autoimmune diseases and inflammatory conditions,

and for the prophylaxis or treatment of psychosomatic, neuropsychiatric and depressive illness, and neurodegenerative diseases such as anxiety, depression, sleep disorders, chronic fatigue, schizophrenia, epilepsy, nutritional disorders, spasm, and chronic pain, and a simple method for the treatment of those disorders.

Solution of the Problem:

According to the invention, the first and second object is solved by use of a compound of the general formula (I), preferably having a glutaminyl or, respectively, a homoglutaminyl residue, each having both an N-unsubstituted α-amino group and an unsubstituted y-amido group, and more preferably by use of a L-α-glutaminyl or, respectively, a L-α-homoglutaminyl residue according to the formulas (NH₂—CO—CH₂—CH₂—CH(NH₂)—CO—) or, respectively, (NH₂—CO—CH₂—CH₂—CH₂—CH(NH₂)—CO—) as a part of the inhibitor molecules of the general formula (I).

The glutaminyl or the homoglutaminyl residue, respectively, renders the inhibitor molecule of the general formula (I) more hydrophilic than ordinary DP IV inhibitors and causes an increase of the transport rate from intestine into blood circulation by the PEPT transporter system. Thus the DP IV inhibitors according to the present invention bear the advantage to exhibit an improved bioavailability after oral uptake compared with ordinary DP IV inhibitors.

A further effect of the introduction of the glutaminyl residue into the DP IV inhibitor molecule concerning the second object of lowered side effects consists of the diminished passage through the blood brain barrier from the circulation into the central nervous system. This leads to a significantly reduced spectrum of undesired side effects of the DP IV inhibitors according to the invention.

Furthermore, it has surprisingly been found that the glutaminyl residue of the DP IV inhibitors of the general formula (I) is metabolized to a cyclic pyroglutaminyl derivative of the general formula (I), which is inactive as DP IV inhibitor in vivo. (see schemes 1 and 2)

The inventors found out that this cyclisation reaction from a glutamin derivative to a pyro-glutamine derivative is accomplished enzymatically, and the responsible enzyme is glutaminyl cyclase. The enzyme glutaminyl cyclase (E.C. 2.3.2.5, abbreviated as QC) is known per se and, furthermore, as being involved in the formation of thyrotropin-releasing hormone and gonadotropin releasing hormone.

A further unexpected result was the finding that substrat specificity of glutaminyl cyclase extends also to homoglutamine. N-terminal homoglutamine as a part of a DP IV inhibitor is metabolisized analogously to glutamin by glutaminyl cyclase to a cyclic pyro-homoglutamine derivative (see reaction schemes 1 and 2 for glutamine and homoglutamine, respectively).

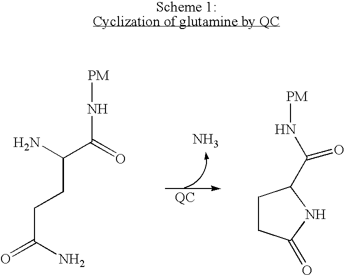

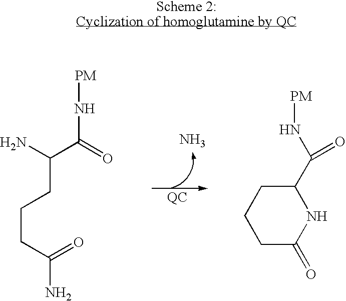

An action of glutaminyl cyclase on low-molecular substances, such as DP IV inhibitors according to the present invention, was not known up to the present invention, which has surprisingly detected the action of QC on DP IV inhibitors containing a glutaminyl residue, especially a L-α-glutaminyl residue at the N-terminus of the DP IV inhibitor according to the present invention. Furthermore, the action of glutaminyl cyclase on DP IV inhibitors containing a homoglutaminyl residue, especially a L-α-homoglutaminyl residue at the N-terminus was unknown up to the present invention.

The ring closure reaction from the open chain glutaminyl derivative being active as a DP IV inhibitor to the cyclic pyroglutaminyl derivative (see scheme 1), which is inactive as a DP IV inhibitor in vivo, is accomplished by the enzyme glutaminyl cyclase (hereinafter abbreviated as QC; E.C. 2.3.2.5) according to the reaction equation mentioned above.

Thus, the third object of the invention is solved by administration of an inhibitor for glutaminyl cyclase (hereinafter abbreviated as QC inhibitor), which prevents the inactivation of the DP IV inhibitor molecule according to the present invention by cyclisation of their glutaminyl or homoglutaminyl residue, respectively. The administration of a glutaminyl cyclase inhibitor in combination with a DP IV inhibitor according to the present invention containing a N-terminal glutaminyl or homoglutaminyl residue, respectively, therefore opens an additional option to control or to prolongate the half life period of the simultaneously administrated DP IV inhibitor, respectively. Therefore a definite and precise adjustment of the half life period of the DP IV inhibitors is possible according to the present invention by a simultaneous administration of both a QC and a DP IV inhibitor.

The DP IV inhibitor according to the present invention, optionally combined with a QC inhibitor, may than act within a definite time period as a medicament for the treatment of conditions mediated by DP IV or DP IV-like enzymes, such as arthritis, obesity, immune and autoimmune disorders, allograft transplantation, cancer, neuronal disorders and dermal diseases. Especially, the DP IV inhibitor according to the present invention, optionally combined with a QC inhibitor, may be used as a medicament for the treatment to improve glucose tolerance by lowering elevated blood glucose levels in response to an oral glucose challenge and, therefore, are useful in treating non-insulin dependent diabetes mellitus (NIDDM; DM Type 2).

Additionally, a synergistic action of DP IV inhibitors togethter with other proteins, which are cleaved and inactivated by DP IV, can be achieved by providing these proteins by a gene therapeutic expression systems in combination with the administration of DP IV inhibitors according to the present invention. These proteins or peptides, respectively, are the glucagon like peptide 1 (GLP-1) and the glucose dependent insulinotropic peptide (GIP) (see WO 03/030946).

Glucagon like peptide 1 (GLP-1) is a peptide synthsized in intestinal L cells in response to nutrient ingestion and promotes nutreint assimiliation via potentiation of glucose dependent insulin secretion. Glucagon like peptide 1 (GLP-1) is produced by proteolytic cleavage of the preproglucagon molecule. Functions of GLP-1 include the enhancement of regulated secretion of insulin from pancreatic β-cells in response to increased blood glucose levels and suppression of glucagon secretion, which together results in a decrease in blood glucose levels without causing hypoglycemia.

Glucagon like peptide 1 (GLP-1) has a extremely short half-life in vivo (<2 minutes). In man, glucagon like peptide 1 (GLP-1), which has an alanine residue at position 2 is quickly inactivated by DP IV, which cleaves specifically dipeptides from peptides and proteins having an alanine or proline residue at position 2. Therefore, it is a further possibility for the treatment of type-2-diabetes and other DP IV related disorders, to provide glucagon like peptide 1 (GLP-1) by a gene therapeutic expression system on one hand, and to prevent the degradation of glucagon like peptide 1 (GLP-1) by DP IV on the other hand by simultaneous administration of DP IV inhibitors according to the present invention. By administrating both GLP-1 and DP IV inhibitors, the half-life of GLP-1 is increased resulting in normalization of glood glucose levels in diabetic patients.

Further, glucose dependent insulinotropic peptide (GIP), a peptide synthesized by duodenum K cells, functions to stimulate insulin release in response to increased blood glucose levels and may also have the advantage of lowering blood lipid levels. Glucose dependent insulinotropic peptide (GIP) directly enhances insulin secretion through a specific GIP receptor expressed on islet β-cells. Unlike GLP-1, GIP has not been demonstrated to improve the phenotype of diabetic patients, although GIP has been shown to enhance insulin-mediated glucose disposal in sheep, rats and mice.

A recent study has demonstrated that, in a similar way to GLP-1, GIP is also inactivated through cleavage at position 2 alanine by DP IV. It has been found, that inhibition of DP IV reduces GIP degradation and potentiates its insulinotropic and antihyperglycemic effects in pigs. Therefore, the expression of GIP in the human body by a gene therapeutic expression system on one side, and the simultaneous administration of a DP IV inhibitor according to the present invention on the other side, is a further possiblity to treat diabetes type 2 and DP IV related disorders.

Additionally, the coexpression of both GIP and GLP-1 by a gene therapeutic expression system on one side, and the simultaneous administration of DP IV inhibitors according to the present invention on the other side, represents a further option for an improved therapy for diabetes type 2 and DP IV related disorders, based on the fact, that the half-life of both GIP and GLP-1 is prolongated by simultaneous administration of DP IV inhibitors according to the present invention. Moreover, all therapies involving a gene therapeutic step may additionally be combined with the administration of a glutaminyl cyclase inhibitor.

### DETAILED DESCRIPTION OF THE INVENTION

The present invention relates to the area of dipeptidyl peptidase IV (DPIV) inhibition and, more particularly, relates to glutaminyl and homoglutaminyl derivatives, wherein a glutaminyl or homoglutaminyl residue, respectively, is bound in a peptid manner to a nitrogen containing residue, pharmaceutical compositions containing said compounds, and the use of said compounds in inhibiting DPIV and DPIV-like enzyme activity.

The present invention provides new DPIV inhibitors, which are effective e.g. in treating conditions mediated by DPIV inhibition, pharmaceutical compositions e.g. useful in inhibiting DPIV and DPIV-like enzyme activity and a method of inhibiting DPIV and DPIV-like enzyme activity.

### BEST EMBODIMENTS FOR CARRYING OUT THE INVENTION

As a first embodiment, the present invention provides a compound of the formula

NR¹R²—C(=EWG1)—(CR³R⁴)—CR⁵R⁶—CRR⁷—CR⁸(NR¹⁰R¹¹)—C(=EWG2)-PM (I)

wherein n is 0 or 1;

wherein R¹, R², R³, R⁴, R⁵, R⁶, R⁷, R⁸, R⁹, R¹⁰, and R¹¹, independently of each other, are

a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R²⁰), a boronic acid group (—B(OH)₂), a cyano group (—C≡N), a carboxylic acid group (—COOH), a carboxylic acid ester group (—COOR²¹), a carboxylic acid anhydride group (—CO—O—CO—R²²), a hydroxamic acid group (—CO—NH(OH)), a N-substituted hydroxamic acid group (—CO—NR²³(OH)), a O-substituted hydroxamic acid group (—CO—NH(OR²⁴)), a carboxamide group (—CO—NH₂), a N-substituted or N,N-disubstituted carboxylic acid amide group, (—CO—NHR²⁵; —CO—NR²⁶R²⁷), an amido group (—HN—CO—R²⁸), a sulfonic acid group (—SO₃H), a sulfonamide group (—SO₂—NH₂), a N-substituted or N,N-disubstituted sulfonamide group (—SO₂—NHR²; —SO₂—NR³R³¹), an amidosulfone group (—NH—SO₂—R³²), a sulfone group (—SO₂—R³³), a phosphoric acid group (—OP(═O)(OH)₂), a phosphoric acid ester group (—OP(═O)(OR³⁴)(OR³⁵)), a phosphonic acid group (—P(═O)(OH)₂), an phosphonic acid ester group (—P(═O)(OR³⁶)(OR³⁷)), a halogen atom, a trifluormethyl group (—CF₃), a thiol group (—SH); a thioether group (—S—R³⁸), a hydroxy group (—OH); an alkoxy group (—O—R³⁹), a tetrazole group, an amino group (—NH₂), or a N-substituted or N,N-disubstituted amino group (—NHR⁴⁰; —N⁴¹R⁴²);

which each independently can be substituted with one or more substituents, which can be the same or different; and,

wherein optionally, any two of the groups R¹, R², R³, R⁴, R⁵, R⁶, R⁷, R⁸, R⁹, R¹⁰, and R¹¹, as well the pairs R²⁶/R²⁷, R³⁰/R³¹, R³⁴/R³⁵, R³⁶/R³⁷ and R⁴¹/R⁴², independenly of each other, may form a part of a ring; and

wherein the substituents R²⁰, R²¹, R²², R²³, R²⁴, R²⁵, R²⁶, R²⁷, R²⁸, R²⁹, R³⁰, R³¹, R³², R³³, R³⁴ R³⁵, R³⁶, R³⁷, R³⁸, R³⁹, R⁴⁰, R⁴¹, and R⁴² independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group; and

wherein EWG1 and EWG2 are each independently an electron withdrawing group and;

wherein the group PM

has the formula (II)

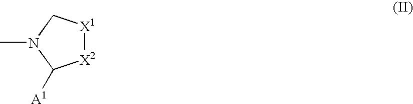

wherein X¹ is CR⁵¹R⁵², O, S, SO, SO₂or NR⁵³; and

wherein X² is CR⁵R⁵, O, S, SO, SO₂, or NR⁵⁶; and

wherein R⁵¹, R⁵², R⁵³, R⁵⁴, R⁵⁵, and R⁵⁶, independently of each other, are

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁶⁰), a boronic acid group (—B(OH)₂), a cyano group (—C≡N), a carboxylic acid group (—COOH), a carboxylic acid ester group (—COOR⁶¹), a carboxylic acid anhydride group (—CO—O—CO—R⁶²), a hydroxamic acid group (—CO—NH(OH)), a N-substituted hydroxamnic acid group (—CO—NR⁶³(OH)), a O-substituted hydroxamic acid group (—CO—NH(OR⁶⁴)), a carboxamide group (—CO—NH₂), a N-substituted or N,N-disubstituted carboxylic acid amide group, (—CO—NHR⁶⁵; —CO—NR⁶⁶, R⁶⁷), an amido group (—HN—CO—R⁶⁸), a sulfonic acid group (—SO₃H), a sulfonamide group (—SO₂—NH₂), a N-substituted or N,N-disubstituted sulfonamide group (—SO₂—NHR⁶⁹; —SO₂ —NR⁷⁰R⁷¹), an amidosulfone group (—NH—SO₂—R⁷²), a sulfone group (—SO₂—R⁷³), a phosphoric acid group (—OP(═O)(OH)₂), a phosphoric acid ester group (—OP(═O)(OR⁷⁴)(OR⁷⁵)), a phosphonic acid group (—P(═O)(OH)₂), an phosphonic acid ester group (—P(═O)(OR⁶)(OR⁷⁷)), a halogen atom, a trifluormethyl group (—CF₃), a thiol group (—SH); a thioether group (—S—R⁷⁸), a hydroxy group (—OH); an alkoxy group (—O—R⁷⁹), a tetrazole group, an amino group (—NH₂), or a N-substituted or N,N-disubstituted amino group (—NHR⁸⁰; —NR⁸¹R⁸²); and

which, independently of each other, can be substituted with one or more substituents, which can be the same or different; and,

wherein optionally, any two of the groups R⁵, R⁵², R⁵³, R⁵⁴, R⁵⁵, and R⁵⁶, if present, as well as the pairs R⁶⁶/R⁶⁷, R⁷⁰/R⁷¹, R⁷⁴/R⁷⁵, R⁷⁶/R⁷⁷ and R⁸¹/R⁸², independently of each other, may form a part of a ring; and

wherein the substituents R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³ R⁷⁴R⁷⁵, R⁷⁶, R⁷⁷, R⁷⁸, R⁷⁹, R⁸⁰, R⁸¹, and R⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group; and

wherein A¹ is

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO-R¹⁰⁰), a boronic acid group (—B(OH)₂), a cyano group (—C≡N), a carboxylic acid group (—COOH), a carboxylic acid ester group (—COOR¹⁰¹), a carboxylic acid anhydride group (—CO—O—CO—R¹⁰²), a hydroxamic acid group (—CO—NH(OH)), a N-substituted hydroxamic acid group (—CO—NR¹⁰³(OH)), a O-substituted hydroxamic acid group (—CO—NH(OR¹⁰⁴)), a carboxamide group (—CO—NH₂), a N-substituted or N,N-disubstituted carboxylic acid amide group, (—CO—NHR¹⁰⁵; —CO—NR¹⁰⁶R¹⁰⁷), an amido group (—HN—CO—R¹⁰⁸), a sulfonic acid group (—SO₃H), a sulfonamide group (—SO₂—NH₂), a N-substituted or N,N-disubstituted sulfonamide group (—SO₂—NHR¹⁰⁹; —SO₂—NR¹¹⁰OR¹¹¹), an amidosulfone group (—NH—SO₂—R¹¹²), a sulfone group (—SO₂—R¹¹³), a phosphoric acid group (—OP(═O)(OH)₂), a phosphoric acid ester group (—OP(═O)(OR¹¹⁴)(OR115)), a phosphonic acid group (—P(═O)(OH)₂), an phosphonic acid ester group (—P(═O)(OR¹⁰⁶)(OR¹⁰⁷)), a halogen atom, a trifluormethyl group (—CF₃), a thiol group (—SH); a thioether group (—S—R¹¹⁸), a hydroxy group (—OH); an alkoxy group (—O—R¹¹⁹), a tetrazole group, an amino group (—NH₂), or a N-substituted or N,N-disubstituted amino group (—NHR¹²⁰; —NR¹²¹R¹²²); and

which, independently of each other, can be substituted with one or more substituents, which can be the same or different; and,

wherein optionally, the pairs R¹⁰⁶/R¹⁰⁷, R¹¹⁰/R¹¹¹, R¹¹⁴/R¹¹⁵, R¹¹⁶/R¹¹⁷ and R¹²¹/R¹²², independently of each other, may form a part of a ring; and

wherein the substituents R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹, R¹¹², R¹¹³, R¹¹⁴, R¹¹⁵, R¹¹⁶, R¹¹⁷, R¹¹⁸, R¹¹⁹, R¹²⁰, R¹²¹, and R¹²², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (III)

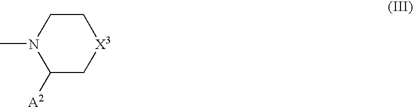

wherein X³ is CR¹³¹R¹³², O, S, SO, SO₂, or NR¹³³; and

wherein R¹³¹, R¹³², and R¹³³, independently of each other, are

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R¹⁴⁰), a boronic acid group (—B(OH)₂), a cyano group (—C≡N), a carboxylic acid group (—COOH), a carboxylic acid ester group (—COOR¹⁴¹), a carboxylic acid anhydride group (—CO—O—CO—R¹⁴²), a hydroxamic acid group (—CO—NH(OH)), a N-substituted hydroxamic acid group (—CO—NR¹⁴³(OH)), a O-substituted hydroxamic acid group (—CO—NH(OR¹⁴⁴)), a carboxamide group (—CO—NH₂), a N-substituted or N,N-disubstituted carboxylic acid amide group, (—CO—NHR¹⁴⁵; —CO—NR¹⁴⁶R¹⁴⁷), an amido group (—HN—CO—R¹⁴⁸), a sulfonic acid group (—SO₃H), a sulfonamide group (—SO₂—NH₂), a N-substituted or N,N-disubstituted sulfonamide group (—SO₂—NHR¹⁴⁹; —SO₂—NR¹⁵⁰R¹⁵¹), an amidosulfone group (—NH—SO₂—R¹⁵²), a sulfone group (—SO₂—R¹⁵³), a phosphoric acid group (—OP(═O)(OH)₂), a phosphoric acid ester group (—OP(═O)(OR¹⁵⁴)(OR¹⁵⁵)), a phosphonic acid group (—P(═O)(OH)₂), an phosphonic acid ester group (—P(═O)(OR¹⁵⁶)(OR⁵⁷)), a halogen atom, a trifluormethyl group (—CF₃), a thiol group (—SH); a thioether group (—S—R¹⁵⁸), a hydroxy group (—OH); an alkoxy group (—O—R¹⁵⁹), a tetrazole group, an amino group (—NH₂), or a N-substituted or N,N-disubstituted amino group (—NHR⁶⁰; —NR⁶¹R¹⁶²); and

which, independently of each other, can be substituted with one or more substituents, which can be the same or different; and,

wherein optionally, the the pair R¹³¹/R¹³², if present, as well the pairs R¹⁴⁶/R¹⁴⁷, R¹⁵⁰/R¹⁵¹, R¹⁵⁴/R¹⁵⁵, R¹⁵⁶/R¹⁵⁷ and R¹⁶¹/R¹⁶², independenly of each other, may form a part of a ring; and

wherein the substituents R¹⁴⁰, R¹⁴¹, R¹⁴², R¹⁴³, R¹⁴⁴, R¹⁴⁵, R¹⁴⁶, R¹⁴⁷, R¹⁴⁸, R¹⁴⁹, R¹⁵⁰, R^{151, R}¹⁵², R¹⁵³, R¹⁵⁴, R¹⁵⁵, R¹⁵⁶, R¹⁵⁷, R¹⁵⁸, R¹⁵⁹, R¹⁶⁰, R¹⁶¹, and R¹⁶², independently of each other are a hydrogen atom (—H), or an alkyl, alkenyl, alkinyl, cycloalkyl, 30 cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

wherein A² is

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R¹⁸⁰), a boronic acid group (—B(OH)₂), a cyano group (—C≡N), a carboxylic acid group (—COOH), a carboxylic acid ester group (—COOR¹⁸¹), a carboxylic acid anhydride group (—CO—O—CO—R¹⁸²), a hydroxamic acid group (—CO—NH(OH)), a N-substituted hydroxamic acid group (—CO—NR¹⁸³(OH)), a O-substituted hydroxamic acid group (—CO—NH(OR¹⁸⁴)), a carboxamide group (—CO—NH₂), a N-substituted or N,N-disubstituted carboxylic acid amide group, (—CO—NHR¹⁸⁵; —CO—NR¹⁸⁶R¹⁸⁷), an amido group (—HN—CO—R¹⁸⁸), a sulfonic acid group (—SO₃H), a sulfonamide group (—SO₂—NH₂), a N-substituted or N,N-disubstituted sulfonamide group (—SO₂—NHR¹⁸⁹; —SO₂—NR¹⁹⁰R¹⁹¹), an amidosulfone group (—NH—SO₂—R¹⁹²), a sulfone group (—SO₂—R⁹³), a phosphoric acid group (—OP(═O)(OH)₂), a phosphoric acid ester group (—OP(═O)(OR¹⁹⁴)(OR¹⁹⁵)), a phosphonic acid group (—P(═O)(OH)₂), an phosphonic acid ester group (—P(═O)(OR¹⁹⁶)(OR¹⁹⁷)), a halogen atom, a trifluormethyl group (—CF₃), a thiol group (—SH); a thioether group (—S—R⁹⁸), a hydroxy group (—OH); an alkoxy group (—O—R¹⁹⁹), a tetrazole group, an amino group (—NH₂), or a N-substituted or N,N-disubstituted amino group (—NHR²⁰⁰; —NR²⁰¹R²⁰²); and

which, independently of each other, can be substituted with one or more substituents, which can be the same or different; and,

wherein optionally, the pairs R¹⁸⁶R¹⁸⁷, R¹⁹⁰/R¹⁹¹, R¹⁹⁴/R¹⁹⁵, R¹⁹⁶/R¹⁹⁷ and R²⁰¹, R²⁰² independenly of each other, may form a part of a ring; and

wherein the substituents R¹⁸⁰, R¹⁸¹, R¹⁸², R¹⁸³, R¹⁸⁴, R¹⁸⁵, R¹⁸⁶, R¹⁸⁷, R¹⁸⁸, R¹⁸⁹, R¹⁹⁰, R¹⁹¹, R¹⁹², R¹⁹³, R¹⁹⁴, R¹⁹⁵, R¹⁹⁶, R¹⁹⁷, R¹⁹⁸, R¹⁹⁹, R²⁰⁰, R²⁰¹, and R²⁰² independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (IV)

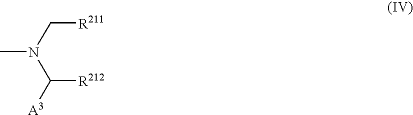

wherein R²¹¹ and R²¹², independently of each other, are

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R²²⁰), a boronic acid group (—B(OH)₂), a cyano group (—C≡N), a carboxylic acid group (—COOH), a carboxylic acid ester group (—COOR²²¹), a carboxylic acid anhydride group (—CO—O—CO—R²²²), a hydroxamnic acid group (—CO—NH(OH)), a N-substituted hydroxamic acid group (—CO—NR²²³ (OH)), a O-substituted hydroxamic acid group (—CO—NH(OR²²⁴)), a carboxamide group (—CO—NH₂), a N-substituted or N,N-disubstituted carboxylic acid amide group, (—CO—NHR²²⁵; —CO—NR²²⁶R²²⁷), an amido group (—HN—CO—R²²⁸), a sulfonic acid group (—SO₃H), a sulfonamide group (—SO₂—NH₂), a N-substituted or N,N-disubstituted sulfonamide group (—SO₂—NHR²²⁹; —SO₂—NR²³⁰R²³¹), an amidosulfone group (—NH—SO₂—R²³²), a sulfone group (—SO₂—R²³³), a phosphoric acid group (—OP(═O)(OH)₂), a phosphoric acid ester group (—OP(═O)(OR²³⁴)(OR²³⁵)), a phosphonic acid group (—P(═O)(OH)₂), an phosphonic acid ester group (—P(═O)(OR²³⁶)(OR²³⁷)), a halogen atom, a trifluormethyl group (—CF₃), a thiol group (—SH); a thioether group (—S—R²³⁸), a hydroxy group (—OH); an alkoxy group (—O—R²³⁹), a tetrazole group, an amino group (—NH₂), or a N-substituted or N,N-disubstituted amino group (—NHR²⁴⁰; —NR²⁴¹R²⁴²); and

which, independently of each other, can be substituted with one or more substituents, which can be the same or different; and,

wherein optionally, the pairs R²²⁶/R²²⁷, R²³⁰/R²³¹, R²³⁴/R²³⁵, R²³⁶/R²³⁷ and R²⁴¹/R²⁴², independenly of each other, may form a part of a ring; and

wherein the substituents R²²⁰, R²²¹, R²²², R²²³, R²²⁴, R²²⁵, R²²⁶, R²²⁷, R²²⁸, R²²⁹, R²³⁰, R²³¹, R²³², R²³³, R²³⁴, R²³⁵, R²³⁶, R²³⁷, R²³⁸, R²³⁹, R²⁴⁰, R²⁴¹, and R²⁴², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

wherein A³ is

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R²⁶⁰), a boronic acid group (—B(OH)₂), a cyano group (—C≡N), a carboxylic acid group (—COOH), a carboxylic acid ester group (—COOR²⁶¹), a carboxylic acid anhydride group (—CO—O—CO—R²⁶²), a hydroxamic acid group (—CO—NH(OH)), a N-substituted hydroxamic acid group (—CO—NR²⁶³(OH)), a O-substituted hydroxamic acid group (—CO—NH(OR²⁶⁴)), a carboxamide group (—CO—NH₂), a N-substituted or N,N-disubstituted carboxylic acid amide group, (—CO—NHR²⁶⁵; —CO-NR²⁶⁶, R²⁶⁷), an amido group (—HN—CO—R²⁶⁸), a sulfonic acid group (—SO₃H), a sulfonamide group (—SO₂—NH₂), a N-substituted or N,N-disubstituted sulfonamide group (—SO₂—NHR²⁶⁹-SO₂—NR²¹⁰R²⁷¹), an amidosulfone group (—NH—SO₂—R²⁷²), a sulfone group (—SO₂—R²⁷³), a phosphoric acid group (—OP(═O)(OH)₂), a phosphoric acid ester group (—OP(═O)(OR²⁷⁴)(OR²⁷⁵)), a phosphonic acid group (—P(═O)(OH)₂), an phosphonic acid ester group (—P(═O)(OR²⁷⁶)(OR²⁷⁷)), a halogen atom, a trifluormethyl group (—CF₃), a thiol group (—SH); a thioether group (—S—R²⁷⁸), a hydroxy group (—OH); an alkoxy group (—O—R²⁷⁹), a tetrazole group, an amino group (—NH₂), or a N-substituted or N,N-disubstituted amino group (—NHR²⁸⁰; —NR²⁸R²⁸²); and

which, independently of each other, can be substituted with one or more substituents, which can be the same or different; and,

wherein optionally, the pairs R²⁶⁶/R²⁶⁷, R²⁷⁰/R²⁷¹, R²⁷⁴/R²⁷⁵, R²⁷⁶/R²⁷⁷ and R²⁸¹/R²⁸², independenly of each other, may form a part of a ring; and

wherein the substituents R²⁶⁰, R²⁶¹, R²⁶², R²⁶³, R²⁶⁴, R²⁶⁵, R²⁶⁶, R²⁶⁷, R²⁶⁸, R²⁶⁹, R²⁷⁰, R²⁷¹, R²⁷², R²⁷³, R²⁷⁴, R²⁷⁵, R²⁷⁶, R²⁷⁷, R²⁷⁸, R²⁷⁹, R²⁸⁰, R²⁸¹, and R²⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (V)

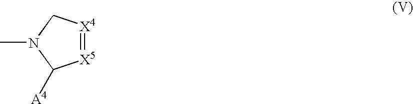

wherein X⁴ iS CR²⁹¹ or N; and

wherein X⁵ is CR²⁹² or N; and

wherein R²⁹¹ and R²⁹², independently of each other, are

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R³⁰⁰), a boronic acid group (—B(OH)₂), a cyano group (—C≡N), a carboxylic acid group (—COOH), a carboxylic acid ester group (—COOR³⁰¹), a carboxylic acid anhydride group (—CO—O—CO—R³⁰²), a hydroxamic acid group (—CO—NH(OH)), a N-substituted hydroxamic acid group (—CO—NR³⁰³(OH)), a O-substituted hydroxamic acid group (—CO—NH(OR³⁰⁴)), a carboxamide group (—CO—NH₂), a N-substituted or N,N-disubstituted carboxylic acid amide group, (—CO—NHR³⁰⁵; —CO—NR³⁰⁶R³⁰⁷), an amido group (—HN—CO—R³⁰⁸), a sulfonic acid group (—SO₃H), a sulfonamide group (—SO₂—NH₂), a N-substituted or N,N-disubstituted sulfonamide group (—SO₂—NHR³⁰⁹; —SO₂—NR³¹⁰R³¹¹), an amidosulfone group (—NH—SO₂—R³¹²), a sulfone group (—SO₂—R³¹³), a phosphoric acid group (—OP(═O)(OH)₂), a phosphoric acid ester group (—OP(═O)(OR³¹⁴)(OR³¹⁵)), a phosphonic acid group (—P(═O)(OH)₂), an phosphonic acid ester group (—P(═O)(OR³¹⁶)(OR³¹⁷)), a halogen atom, a trifluormethyl group (—CF₃), a thiol group (—SH); a thioether group (—S—R³¹⁸), a hydroxy group (—OH); an alkoxy group (—O—R³¹⁹), a tetrazole group, an amino group (—NH₂), or a N-substituted or N,N-disubstituted amino group (—NHR³²⁰; —NR³²¹R³²²); and

which, independently of each other, can be substituted with one or more substituents, which can be the same or different; and,

wherein optionally, the the pair R²⁹¹/R²⁹², if present, as well the pairs R³⁰⁶/R³⁰⁷, R³¹⁰/R³¹¹, R³¹⁴/R³¹⁵, R³¹⁶/R³¹⁷ and R³²¹/R³²², independenly of each other, may form a part of a ring; and

wherein the substituents R³⁰⁰, R³⁰¹, R³⁰², R³⁰³, R³⁰⁴, R³⁰⁵, R³⁰⁶, R³⁰⁷, R³⁰⁸, R³⁰⁹, R³¹⁰, R³¹¹, R³¹², R³¹³, R³¹⁴, R³¹⁵, R³¹⁶, R³¹⁷, R³¹⁸, R³¹⁹, R³²⁰, R³²¹, and R³²², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

wherein A⁴ is

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁴⁰), a boronic acid group (—B(OH)₂), a cyano group (—C≡N), a carboxylic acid group (—COOH), a carboxylic acid ester group (—COOR³⁴¹), a carboxylic acid anhydride group (—CO—O—CO—R³⁴²), a hydroxamic acid group (—CO—NH(OH)), a N-substituted hydroxamic acid group (—CO—NR³⁴³(OH)), a O-substituted hydroxamic acid group (—CO—NH(OR³⁴⁴)), a carboxamide group (—CO—NH₂), a N-substituted or N,N-disubstituted carboxylic acid amide group, (—CO—NHR³⁴⁵; —CO—NR³⁴⁶R³⁴⁷), an amido group (—HN—CO—R³⁴⁸), a sulfonic acid group (—SO₃H), a sulfonamide group (—SO₂—NH₂), a N-substituted or N,N-disubstituted sulfonamide group (—SO₂—NHR³⁴⁹; —SO₂—NR³⁵⁰R³⁵¹), an amidosulfone group (—NH—SO₂—R⁵²), a sulfone group (—SO₂—R³⁵³), a phosphoric acid group (—OP(═O)(OH)₂), a phosphoric acid ester group (—OP(═O)(OR³⁵⁴)(OR³⁵⁵)) a phosphonic acid group (—P(═O)(OH)₂), an phosphonic acid ester group (—P(═O)(OR³⁵⁶)(OR³⁵⁷)), a halogen atom, a trifluormethyl group (—CF₃), a thiol group (—SH); a thioether group (—S—R³⁵⁸), a hydroxy group (—OH); an alkoxy group (—O—R³⁵⁹), a tetrazole group, an amino group (—NH₂), or a N-substituted or N,N-disubstituted amino group (—NHR³⁶⁰; —NR³⁶¹R³⁶²); and

which, independently of each other, can be substituted with one or more substituents, which can be the same or different; and,

wherein optionally, the pairs R³⁴⁶/R³⁴⁷, R³⁵⁰/R³⁵¹, R³⁵⁴/R³⁵⁵/R³⁵⁶/R³⁵⁷ and R³⁶¹/R³⁶², independenly of each other, may form a part of a ring; and

wherein the substituents R³⁴⁰, R³⁴¹, R³⁴², R³⁴³, R³⁴⁵, R³⁴⁶, R³⁴⁷, R³⁴⁸, R³⁴⁹, R³⁵⁰, R³⁵¹, R³⁵², R³⁵³, R³⁵⁴, R³⁵⁵, R³⁵⁶, R³⁵⁷, R³⁵⁸, R³⁵⁹, R³⁶⁰, R³⁶¹, and R³⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (VI)

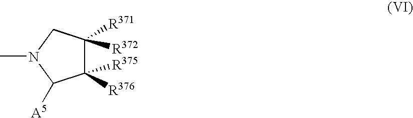

wherein R³⁷¹, R³⁷², R³⁷⁵ and R³⁷⁶, independently of each other, a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R³⁸⁰), a boronic acid group (—B(OH)₂), a cyano group (—C≡N), a carboxylic acid group (—COOH), a carboxylic acid ester group (—COOR³⁸¹), a carboxylic acid anhydride group (—CO—O—CO—R³⁸²), a hydroxamic acid group (—CO—NH(OH)), a N-substituted hydroxamic acid group (—CO—NR³⁸³(OH)), a O-substituted hydroxamic acid group (—CO—NH(OR³⁸⁴)), a carboxamide group (—CO—NH₂), a N-substituted or N,N-disubstituted carboxylic acid amide group, (—CO—NHR³⁸⁵; —CO—NR³⁸⁶R³⁸⁷), an amido group (—HN—CO—R³⁸⁸), a sulfonic acid group (—SO₃H), a sulfonamide group (—SO₂—NH₂), a N-substituted or N,N-disubstituted sulfonamide group (—SO₂—NHR³⁸⁹; —SO₂—N³⁹⁰R³⁹¹), an amidosulfone group (—NH—SO₂—R¹⁹²), a sulfone group (—SO₂—R³⁹³), a phosphoric acid group (—OP(═O)(OH)₂), a phosphoric acid ester group (—OP(═O)(OR³⁹⁴)(OR³⁹⁵)), a phosphonic acid group (—P(═O)(OH)₂), an phosphonic acid ester group (—P(═O)(OR³⁹⁶)(OR³⁹⁷)), a halogen atom, a trifluormethyl group (—CF₃), a thiol group (—SH); a thioether group (—S—R³⁹⁸), a hydroxy group (—OH); an alkoxy group (—O—R³⁹⁹), a tetrazole group, an amino group (—NH₂), or a N-substituted or N,N-disubstituted amino group (—NHR⁴o; —NR⁴⁰¹R⁴⁰²); and

which, independently of each other, can be substituted with one or more substituents, which can be the same or different; and,

wherein optionally, any two of the groups R³⁷¹, R³⁷², R³⁷⁵, and R³⁷⁶, as well as the pairs R³⁸⁶/R³⁸⁷, R³⁹⁰/R³⁹¹, R³⁹⁴/R³⁹⁵, R³⁹⁶/R³⁹⁷ and R⁴⁰¹/R⁴⁰², independenly of each other, may form a part of a ring; and

wherein the substituents R³⁸⁰, R³⁸¹, R³⁸², R³⁸³, R³⁸⁴, R³⁸⁵, R³⁸⁶, R³⁸⁷, R³⁸⁸, R³⁸⁹, R³⁹⁰, R³⁹¹, R³⁹², R³⁹³, R³⁹⁴, R³⁹⁵, R³⁹⁶, R³⁹⁷, R³⁹⁸, R³⁹⁹, R⁴⁰⁰, R⁴⁰¹, and R⁴⁰², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group; or

alternatively; the two groups R³⁷¹ and R³⁷² can be together an oxo (═O) or hydroxyimino (═N—OH) group; and

alternatively; the two groups R³⁷⁵ and R³⁷⁶ can be together an oxo (═O) or hydroxyimino (═N—OH) group; and

wherein A⁵ is

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁴²⁰), a boronic acid group (—B(OH)₂), a cyano group (—C≡N), a carboxylic acid group (—COOH), a carboxylic acid ester group (—COOR⁴²¹), a carboxylic acid anhydride group (—CO—O—CO—R⁴²²), a hydroxamic acid group (—CO—NH(OH)), a N-substituted hydroxamic acid group (—CO—NR⁴²¹ (OH)), a O-substituted hydroxamnic acid group (—CO—NH(OR⁴²⁴)), a carboxamide group (—CO—NH₂), a N-substituted or N,N-disubstituted carboxylic acid amide group, (—CO—NHR⁴²⁵; —CO—NR⁴²⁰R⁴²¹), an amido group (—HN—CO—R⁴²⁸), a sulfonic acid group (—SO₃H), a sulfonamide group (—SO₂—NH₂), a N-substituted or N,N-disubstituted sulfonamide group (—SO₂—NHR⁴²⁹;

SO₂—NR⁴³⁰R⁴³¹), an amidosulfone group (—NH—SO₂—R⁴³²), a sulfone group (—SO₂—R⁴³³), a phosphoric acid group (—OP(═O)(OH)₂), a phosphoric acid ester group (—OP(═O)(OR⁴³⁴)(OR⁴³⁵)), a phosphonic acid group (—P(═O)(OH)₂), an phosphonic acid ester group (—P(═O)(OR⁴³⁶)(OR⁴³⁷)), a halogen atom, a trifluormethyl group (—CF₃), a thiol group (—SH); a thioether group (—S—R⁴³⁸), a hydroxy group (—OH); an alkoxy group (—O—R⁴³⁹), a tetrazole group, an amino group (—NH₂), or a N-substituted or N,N-disubstituted amino group (—NHR⁴⁴⁰; —NR⁴⁴¹R⁴⁴²); and

which, independently of each other, can be substituted with one or more substituents, which can be the same or different; and,

wherein optionally, the pairs R⁴²⁶/R⁴²⁷, R⁴³⁰/R⁴³¹, R⁴³⁴/R⁴³⁵, R⁴³⁶/R⁴³⁷ and R⁴⁴¹/R⁴⁴², independenly of each other, may form a part of a ring; and

wherein the substituents R⁴²⁰, R⁴²¹, R⁴²², R⁴²³, R⁴²⁴, R⁴²⁵, R⁴²⁶, R⁴²⁷, R⁴²⁸, R⁴²⁹, R⁴³⁰R⁴³¹, R⁴³², R⁴³³, R⁴³⁴, R⁴³⁵, R⁴³⁶, R⁴³⁷, R⁴³⁸, R⁴³⁹, R⁴⁴⁰, R⁴⁴¹ and R⁴⁴², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (VII)

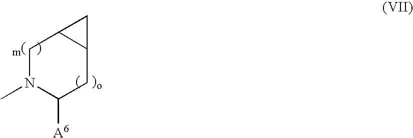

wherein m is equal to 1 or 2, and o is equal to 1 or 2, and m or o can be 0;

wherein A⁶ is a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁴⁶⁰), a boronic acid group (—B(OH)₂), a cyano group (—C≡N), a carboxylic acid group (—COOH), a carboxylic acid ester group (—COOR⁴⁶¹), a carboxylic acid anhydride group (—CO—O—CO—R⁴⁶²), a hydroxamic acid group (—CO—NH(OH)), a N-substituted hydroxamic acid group (—CO—NR⁴⁶³(OH)), a O-substituted hydroxamic acid group (—CO—NH(OR⁴⁶⁴)), a carboxamide group (—CO—NH₂), a N-substituted or N,N-disubstituted carboxylic acid amide group, (—CO—NHR⁴⁶⁵; —CO—NR⁴⁶⁶R⁴⁶⁷), an amido group (—HN—CO—R⁴⁶⁸), a sulfonic acid group (—SO₃H), a sulfonamide group (—SO₂—NH₂), a N-substituted or N,N-disubstituted sulfonamide group (—SO₂—NHR⁴⁶; —SO₂—NR⁴⁷⁰⁴⁷¹), an amidosulfone group (—NH—SO₂—R⁴⁷²), a sulfone group (—SO₂—R⁴⁷³), a phosphoric acid group (—OP(═O)(OH)₂), a phosphoric acid ester group (—OP(═O)(OR⁴⁷⁴)(OR⁴⁷⁵)), a phosphonic acid group (—P(═O)(OH)₂), an phosphonic acid ester group (—P(═O)(OR⁴⁷⁶)(OR⁴⁷⁷)), a halogen atom, a trifluormethyl group (—CF₃), a thiol group (—SH); a thioether group (—S—R⁴⁷⁸), a hydroxy group (—OH); an alkoxy group (—O—R⁴⁷⁹), a tetrazole group, an amino group (—NH₂), or a N-substituted or N,N-disubstituted amino group (—NHR⁴⁸⁰; —NR⁴⁸¹R⁴⁸²);

which, independently of each other, can be substituted with one or more substituents, which can be the same or different; and,

wherein optionally, the pairs R^{466/467}, R⁴⁴⁰/R⁴⁷¹, R⁴⁷⁴/R⁴⁷⁵, R⁴⁷⁶/R⁴⁷⁷ and R⁴⁸¹/R⁴⁸² independenly of each other, may form a part of a ring; and

wherein the substituents R⁴⁶⁰, R⁴⁶¹, R⁴⁶², R⁴⁶³, R⁴⁶⁴, R⁴⁶⁵, R⁴⁶⁶, R⁴⁶⁷, R⁴⁶⁸, R⁴⁶⁹., R⁴⁷⁰, R⁴⁷¹, R⁴⁷², R⁴⁷³, R⁴⁷⁴, R⁴⁷⁵, R⁴⁷⁶, R⁴⁷⁷, R⁴⁷⁸, R⁴⁷⁹, R⁴⁸⁰, R⁴⁸¹ and R⁴⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (VM)

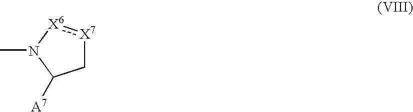

wherein X⁶ is selected from CR⁴⁹⁰R⁴⁹¹, O, S or NR⁴⁹², when the bond between x6 and X⁷ is a single bond; and

wherein X⁷ is selected from CR⁴⁹³R^{494, 0}, S, or NR⁴⁹⁵, when the bond between X⁶ and X⁷ is a single bond;

or alternatively,

wherein X is selected from CR⁴⁹⁶ or N, when the bond between X⁶ and X⁷ is a double bond; and

wherein X⁷ is selected from CR⁴⁹⁷ or N, when the bond between X⁶ and X⁷ is a double bond; and

wherein R⁴⁹⁰, R⁴⁹¹, R⁴⁹², R⁴⁹³, R⁴⁹⁴, R⁴⁹⁵, R⁴⁹⁶, and R⁴⁹⁷, independently of each other, are a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁵⁰⁰), a boronic acid group (—B(OH)₂), a cyano group (—C≡N), a carboxylic acid group (—COOH), a carboxylic acid ester group (—COOR⁵⁰¹), a carboxylic acid anhydride group (—CO—O—CO—R⁵⁰²), a hydroxamic acid group (—CO—NH(OH)), a N-substituted hydroxamic acid group (—CO—NR⁵⁰³ (OH)), a O-substituted hydroxamic acid group (—CO—NH(OR⁵⁰⁴)), a carboxamide group (—CO—NH₂), a N-substituted or N,N-disubstituted carboxylic acid amide group, (—CO—NHR⁵⁰⁵; —CO—NR⁵⁰⁶R⁵⁰⁷), an amido group (—HN—CO—R⁵⁰⁸), a sulfonic acid group (—SO₃H), a sulfonamide group (—SO₂—NH₂), a N-substituted or N,N-disubstituted sulfonamide group (—SO₂—NHR⁵¹³; —SO₂—NR⁵¹⁰R⁵¹¹), an amidosulfone group (—NH—SO₂—R⁵¹²), a sulfone group (—SO₂—R⁵¹³), a phosphoric acid group (—OP(═O)(OH)₂), a phosphoric acid ester group (—OP(═O)(OR⁵¹⁴)(OR⁵¹⁵)), a phosphonic acid group (—P(═O)(OH)₂), an phosphonic acid ester group (—P(═O)(OR⁵¹⁶)(OR⁵¹⁷)), a halogen atom, a trifluormethyl group (—CF₃), a thiol group (—SH); a thioether group (—S—R⁵¹⁸), a hydroxy group (—OH); an alkoxy group (—O—R⁵¹⁹), a tetrazole group, an amino group (—NH₂), or a N-substituted or N,N-disubstituted amino group (—NHR⁵²⁰; —NR⁵²¹R⁵²²); and

which, independently of each other, can be substituted with one or more substituents, which can be the same or different; and, wherein optionally, any two the groups R⁴⁹⁰, R⁴⁹¹, R⁴⁹², R⁴⁹³, R⁴⁹⁴, R⁴⁹⁵, R⁴⁹⁶, and R⁴⁹⁷, if present, as well as the pairs R⁵⁰⁶/R⁵⁰⁷, R⁵¹⁰/R⁵¹¹, R⁵¹⁴/R⁵¹⁵, R⁵¹⁶/R⁵¹⁷ and R⁵²¹/R⁵²², independenly of each other, may form a part of a ring; and

wherein the substituents R⁵⁰⁰, R⁵⁰¹, R⁵⁰², R⁵⁰³, R⁵⁰⁴, R⁵⁰⁵, R⁵⁰⁶, R⁵⁰⁷, R⁵⁰⁸, R⁵⁰⁹, R⁵¹⁰, R⁵¹¹, R⁵¹², R⁵¹³, R⁵¹⁴, R⁵¹⁵, R⁵¹⁶, R⁵¹⁷, R⁵¹⁸, R⁵¹⁹, R⁵²⁰, R⁵²¹, and R⁵²², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, 10 heterocycloalkenyl, aryl, heteroaryl, aryl-alkyl, heteroaryl-alkyl, aryl-heteroalkyl, heteroaryl-heteroalkyl group; and

wherein A⁷ is

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁵⁴⁰), a boronic acid group (—B(OH)₂), a cyano group (—C≡N), a carboxylic acid group (—COOH), a carboxylic acid ester group (—COOR⁵⁴¹), a carboxylic acid anhydride group (—CO—O—CO—R⁵⁴²), a hydroxamic acid group (—CO—NH(OH)), a N-substituted hydroxamic acid group (—CO—NR⁵⁴³ (OH)), a O-substituted hydroxamic acid group (—CO—NH(OR⁵⁴⁴)), a carboxamide group (—CO—NH₂), a N-substituted or N,N-disubstituted carboxylic acid amide group, (—CO—NHR⁵⁴⁵; —CO—NR⁵⁴⁶R⁵⁴⁷), an amido group (—HN—CO—R⁵⁴⁸), a sulfonic acid group (—SO₃H), a sulfonamide group (—SO₂—NH₂), a N-substituted or N,N-disubstituted sulfonamide group (—SO₂—NHR^{549; 25}-SO₂—NR⁵⁵⁰R⁵⁵¹), an amidosulfone group (—NH—SO₂—R⁵⁵²), a sulfone group (—SO₂—R⁵⁵³), a phosphoric acid group (—OP(═O)(OH)₂), a phosphoric acid ester group (—OP(═O)(OR⁵⁵⁴)(OR⁵⁵⁵)), a phosphonic acid group (—P(═O)(OH)₂), an phosphonic acid ester group (—P(═O)(OR⁵⁵⁶)(OR⁵⁵⁷)), a halogen atom, a trifluormethyl group (—CF₃), a thiol group (—SH); a thioether group (—S—R⁵⁵⁸), a hydroxy group (—OH); an alkoxy group (—O-30, R⁵⁵⁹), a tetrazole group, an amino group (—NH₂), or a N-substituted or N,N-disubstituted amino group (—NHR⁵⁶⁰; —NR⁵⁶¹R⁵⁶²); and

which, independently of each other, can be substituted with one or more substituents, which can be the same or different; and,

wherein optionally, the pairs R⁵⁴⁶/R⁵⁴⁷, R⁵⁵⁰/R⁵, R⁵⁵⁴¹, R⁵⁵⁵, R⁵⁵⁶/R⁵⁵⁷ and R⁵⁶¹/R⁵⁶², independenly of each other, may form a part of a ring; and

wherein the substituents R⁵⁴⁰, R⁵⁴¹, R⁵⁴², R⁵⁴³, R⁵⁴⁴, R⁵⁴⁵, R⁵⁴⁶, R⁵⁴⁷, R⁵⁴⁸, R⁵⁴⁹, R⁵⁵⁰, R⁵⁵¹, R⁵⁵², R⁵⁵³, R⁵⁵⁴, R⁵⁵⁵, R⁵⁵⁶, R⁵⁵⁷, R⁵⁵⁸, R⁵⁵⁹, R⁵⁶⁰, R⁵⁶¹, and R⁵⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (IX) or (IXa)

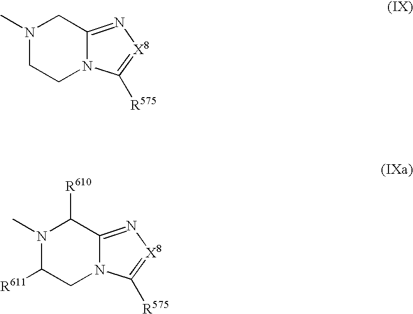

wherein X⁸ is N or CR⁵⁷⁰; and

wherein R⁵⁷⁰, R⁵⁷⁵, R⁶¹⁰ and R⁶¹¹ independently of each other, are a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁵⁸⁰), a boronic acid group (—B(OH)₂), a cyano group (—C≡N), a carboxylic acid group (—COOH), a carboxylic acid ester group (—COOR⁵⁸¹), a carboxylic acid anhydride group (—CO—O—CO—R⁵⁸²), a hydroxamic acid group (—CO—NH(OH)), a N-substituted hydroxamic acid group (—CO—NR⁵⁸³(OH)), a O-substituted hydroxamic acid group (—CO—NH(OR⁵⁸⁴)), a carboxamide group (—CO—NH₂), a N-substituted or N,N-disubstituted carboxylic acid amide group, (—CO—NHR⁵⁸⁵; —CO—NR⁵⁸⁶R⁵⁸⁷), an amido group (—HN—CO—R⁵⁸⁸), a sulfonic acid group (—SO₃H), a sulfonamide group (—SO₂—NH₂), a N-substituted or N,N-disubstituted sulfonamide group (—SO₂—NHR⁵⁸⁹; —SO₂—NR⁵⁹⁰R⁵⁹¹), an amidosulfone group (—NH—SO₂—R⁵⁹²), a sulfone group (—SO₂—R⁵⁹³), a phosphoric acid group (—OP(═O)(OH)₂), a phosphoric acid ester group (—OP(═O)(OR⁵⁹⁴)(OR⁵⁹⁵)), a phosphonic acid group (—P(═O)(OH)₂), an phosphonic acid ester group (—P(═O)(OR⁵⁹⁶)(OR⁵⁹⁷)), a halogen atom, a trifluormethyl group (—CF₃), a thiol group (—SH); a thioether group (—S—R⁵⁹⁸), a hydroxy group (—OH); an alkoxy group (—O—R⁵⁹⁹), a tetrazole group, an amino group (—NH₂), or a N-substituted or N,N-disubstituted amino group (—NHR⁶⁰⁰; —NR⁶⁰¹R⁶⁰²);

which, independently of each other, can be substituted with one or more substituents, which can be the same or different; and,

wherein optionally, the pairs R⁵⁷⁰/R⁵⁷⁵, if present, as well as the pairs R⁵⁸⁶/R⁵⁸⁷, R⁵⁹⁰/R⁵⁹¹, R⁵⁹⁴/R⁵⁹⁵, R⁵⁹⁶/R⁵⁹⁷ and R⁶⁰¹/R⁶⁰², independenly of each other, may form a part of a ring; and

wherein the substituents R⁵⁸⁰, R⁵⁸¹, R⁵⁸², R⁵⁸³, R⁵⁸⁴, R⁵⁸⁵, R⁵⁸⁶, R⁵⁸⁷, R⁵⁸⁸, R⁵⁸⁹, R⁵⁹⁰, R⁵⁹¹, R⁵⁹², R⁵⁹³, R⁵⁹⁴, R⁵⁹⁵, R⁵⁹⁶, R⁵⁹⁷, R⁵⁹⁸, R⁶⁰⁰, R⁶⁰¹, and R⁶⁰², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (X)

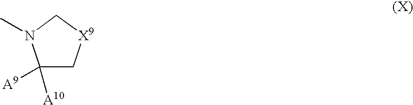

wherein the groups X⁹ is CR⁹⁰⁰R⁹⁰¹, S, SO, SO₂or NR⁹⁰²

wherein R⁹⁰⁰, R⁹⁰¹ and R⁹⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR⁹¹⁰R⁹¹¹.

wherein A⁹ and A¹⁰ are, independently of each other, selected from hydrogen, cyano, —C(═O)NR⁹¹²R⁹¹³, or C₁, C₂, C₃, C₄, C₅or C₆alkyl, which is linear or branched and is optionally substituted with 1, 2, 3, 4, or 5 halogens;

wherein

R⁹¹⁰ and R⁹¹², are, independently of each other, selected from hydrogen, or C₁, C₂, C₃, C₄, C₅or C₆alkyl, which is linear or branched and is optionally substituted with 1, 2, 3, 4, or 5 halogens; and

R⁹¹¹ and R⁹¹³, are, independently of each other, selected from the group consisting of

(1) phenyl, which is optionally substituted with 1, 2, 3, 4, or 5, substituents independently selected from halogen and R⁹²⁰;

(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of

(a) hydroxy,

(b) —COOH,

(c) —COO(C₁, C₂, C₃, C₄, C₅or C₆alkyl), i.e. ester,

(d) phenyl,

(e) naphthyl,

(f) C₃, C₄, C₅or C₆cycloalkyl,

(g) a 5- or 6 membered heterocycle which may be saturated or unsaturated comprising 1, 2, 3, or 4 heteroatoms independently selected from nitrogen, oxygen or sulfur;

(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;

wherein said C₃, C₄, C₅or C₆cycloalkyl, phenyl, naphthyl, are optionally substituted with 1, 2, 3, 4, or 5 substituents independently selected from halogen and R⁹²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R⁹²⁰; and

(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, Cn, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;

wherein R⁹²⁰ is selected from the group consisting of:

(1) hydroxy;

(2) cyano;

(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;

(4) C₁, C₂, C₃, C₄, Cs, C₆, C₇, C₈, Cg or C₁₀alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from

(a) hydroxy;

(b) —COOH;

(c) —COO(C₁, C₂, C₃, C₄, C₅or C₆alkyl) i.e. ester, which may linear or branched and is optionally substituted with 1, 2, 3, 4, or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR⁹²⁵R⁹²⁵;

(g) —SO₂NR⁹²⁵R⁹²⁵;

(h) —NR⁹²⁵—C(═O)R⁹²⁵

(i) —NR⁹²⁵C(═O)NR⁹²⁵, R⁹²⁵

(j) —NR⁹²⁵COOR⁹³⁰

(k) —O—CO—R⁹³⁰

(l) —O—CO—NR⁹²⁵R⁹²⁵;

(m) —NR⁹²SO₂R⁹³⁰;

(n) NR⁹²⁵R⁹²⁵;

(o) phenyl which is optionally substituted with 1, 2, 3, 4, or 5 group independently selected from halogen, hydroxy, C₁, C₂, C₃, C₄, Cs or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(5) OC₁, OC₂, OC₃, OC₄, OC₅, OC₆, OC₇, OC₈, OC₉or OC₁₀alkyl, which is linear or branched and is optionally substituted with 0, 1, 2, 3, 4, or 5 halogen atoms and 0, 1, or 2 substitutents selected from

(a) hydroxy;

(b) —COOH;

(c) —COO(C₁, C₂, C₃, C₄, C₅or C₆alkyl) i.e. ester, which may be linear or branched and is optionally substituted with 1, 2, 3, 4 or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, Cs or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR⁹²⁵, R⁹²⁵;

(g) —SO₂NR⁹²⁵R⁹²⁵;

(h) —NR⁹²⁵—C(═O)R⁹²⁵

(i) —NR⁹²¹—C(═O)NR⁹²⁵R⁹²⁵

(o) —NR⁹²⁵ COOR⁹³⁰

(k) —O—CO—R⁹³⁰

(l) —O—CO—NR⁹²⁵R¹⁰²⁵

(m) —NR⁹²⁵SO₂R⁹³⁰;

(n) NR⁹²⁵R⁹²⁵;

(0) phenyl, which is optionally substituted with 1, 2, 3, 4, or 5 groups independently selected from halogen, hydroxy, C₁, C₂, C₃, C₄, Cs or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(6) —COOH;

(7) —COO(C₁, C₂, C₃, C₄, C₅ or C₆alkyl) i.e. ester, which may be linear or branched and is optionally substituted with 1, 2, 3, 4, 5 halogens;

(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.

(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 0.1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(10) —CONR⁹²⁵R⁹²⁵;

(11) —SO₂NR⁹²⁵R⁹²⁵;

(12) —NR⁹²⁵—C(═O)R⁹²⁵

(13)—NR⁹²⁵—C(═O)NR⁹²⁵R⁹²⁵;

(14) —NR⁹²⁵COOR⁹³⁰

(15)—O—CO—R⁹³⁰ (16)—O—CO—NR⁹²⁵, R⁹²⁵;

(17) —NR⁹²⁵SO₂R⁹³⁰;

(18)—NR⁹²⁵R⁹²⁵;

(19) phenyl, which is optionally substituted with 1, 2, 3, 4, or 5 group independently selected from halogen, hydroxy, C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;

wherein R⁹³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.

wherein R²⁵ is selected from R⁹³⁰ and hydrogen.

wherein the group PM

has the formula (XI)

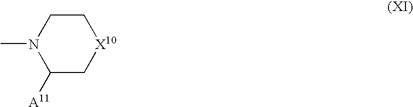

wherein the groups X¹⁰ is CR¹⁰⁰⁰R¹⁰⁰¹, S. SO, SO₂or NR¹⁰⁰²

wherein R¹⁰⁰⁰, R¹⁰⁰¹ and R¹⁰⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR⁹¹⁰R⁹¹¹.

and A¹¹ is selected from

hydrogen, cyano, —C(═O)NR¹⁰¹²R¹⁰¹³ or C₁, C₂, C₃, C₄, C₅or C₆alkyl, which is linear or branched and is optionally substituted with 1, 2, 3, 4, or 5 halogens;

wherein

R¹⁰¹⁰ and R¹⁰¹², are, independently of each other, selected from hydrogen, or C₁, C₂, C₃, C₄, C₅or C₆alkyl, which is linear or branched and is optionally substituted with 1, 2, 3, 4, or 5 halogens; and

R¹⁰¹¹ and R¹⁰¹³, are, independently of each other, selected from the group consisting of

(1) phenyl, which is optionally substituted with 1, 2, 3, 4, or 5, substituents independently selected from halogen and R¹⁰²⁰

(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of

(a) hydroxy,

(b) —COOH,

(c) —COO(C₁, C₂, C₃, C₄, C₅or C₆alkyl), i.e. ester,

(d) phenyl,

(e) naphthyl,

(f) C₃, C₄, C₅or C₆cycloalkyl,

(g) a 5- or 6 membered htereocycle which may be saturated or unsaturated comprising 1, 2, 3, or 4 heteroatoms independently selected from nitrogen, oxygen or sulfur;

(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;

wherein said C₃, C₄, C₅or C₆cycloalkyl, phenyl, naphthyl, are optionally substituted with 1, 2, 3, 4, or 5 substituents independently selected from halogen and R¹⁰²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R¹⁰²⁰; and

(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;

wherein R¹⁰²⁰ is selected from the group consisting of:

(1) hydroxy;

(2) cyano;

(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;

(4) C₁, C₂, C₃, C₄, C₅, C₆, C₇, C₈, C₉ or C₁₀ alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from

(a) hydroxy;

(b) —COOH;

(c) —COO(C₁, C₂, C₃, C₄, C₅or C₆alkyl) i.e. ester, which may linear or branched and is optionally substituted with 1, 2, 3, 4, or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR¹⁰²⁵R¹⁰²⁵R¹⁰²⁵;

(g) —SO₂NR¹⁰²⁵R¹⁰²⁵;

(h) —NR¹⁰²⁵—C(═O)R¹⁰²⁵

(i) —NR¹⁰²⁵—C(═O)NR¹⁰²⁵R¹⁰²⁵;

(j) —NR¹⁰²⁵COOR¹⁰³⁰

(k) —O—CO—R¹⁰³⁰

(l) —O—CO—NR¹⁰²⁵R¹⁰²⁵;

(m) —NR¹⁰²⁵SO₂R¹⁰³⁰;

(n) _NR¹⁰²⁵, R¹⁰²⁵;

(o) phenyl which is optionally substituted with 1, 2, 3, 4, or 5 group independently selected from halogen, hydroxy, C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅ or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(5) OC₁, OC₂, OC₃, OC₄, OC₅, OC₆, OC₇, OC₈, OC₉or OC₁₀alkyl, which is linear or branched and is optionally substituted with 0, 1, 2, 3, 4, or 5 halogen atoms and 0, 1, or 2 substitutents selected from

(a) hydroxy;

(b) —COOH;

(c) —COO(C₁, C₂, C₃, C₄, C₅or C₆alkyl) i.e. ester, which may be linear or branched and is optionally substituted with 1, 2, 3, 4 or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR¹⁰²⁵R¹⁰²⁵;

(g) —SO₂NR¹⁰²⁵R¹⁰²⁵;

(h) —NR¹⁰²⁵—C(═O)R¹⁰²⁵

(i) —NR¹⁰²⁵—C(═O)NR¹⁰²⁵, R¹⁰²⁵

(j) —NR¹⁰²⁵ COOR¹⁰³⁰

(k) —O—CO—R¹⁰³⁰

(l) —O—CO—NR¹⁰²⁵R¹⁰²⁵;

(m) —NR¹⁰²⁵, R⁰³⁰;

(n) _NR¹⁰²⁵R¹⁰²⁵;

(o) phenyl, which is optionally substituted with 1, 2, 3, 4, or 5 groups independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(6) —COOH;

(7) —COO(C₁, C₂, C₃, C₄, C₅or C₆alkyl) i.e. ester, which may be linear or branched and is optionally substituted with 1, 2, 3, 4, 5 halogens;

(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.

(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(10)—CONR¹⁰²⁵R¹⁰²⁵R¹⁰²⁵;

(11) —SO₂NR¹⁰²⁵R¹⁰²⁵;

(12) —NR¹⁰²⁵—C(═O)R¹⁰²⁵

(13) —NR¹⁰²⁵—C(═O)NR¹⁰²⁵R¹⁰²⁵

(14) —NR⁹²⁵COOR¹⁰³⁰

(15) —O—CO—R¹⁰³⁰

(16) —O—CO—NR¹⁰²⁵R¹⁰²⁵;

(17) —NR¹⁰²⁵ SO₂R¹⁰³⁰;

(18)—NR¹⁰²⁵R¹⁰²⁵.

(19) phenyl, which is optionally substituted with 1, 2, 3, 4, or 5 group independently selected from halogen, hydroxy, C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, Cs or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;

wherein R¹⁰³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄Cs or C₆ cycloalkyl, are optionally substituted with 1, 2, 3, 4, or 5 substituents, independently selected from halogen, OH, C₁, C₂, C₃, C₄, or Cs alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.

wherein R¹⁰²⁵ is selected from R¹⁰³⁰ and hydrogen.

or wherein the group PM

has the formula (XII)

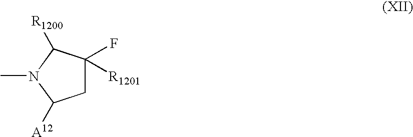

wherein the groups R¹²⁰¹ is hydrogen or fluoro.

wherein R¹²⁰⁰ und A¹² is selected from hydrogen and cyano, and the other is hydrogen.

or wherein the group PM

has the formula XIII:

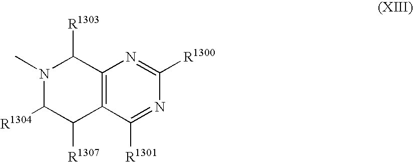

wherein:

R¹³⁰⁰ and R¹³⁰¹ are independently selected from the group consisting of:

(1) hydrogen,

(2) CN,

(3) C₁₋₁₀alkyl, which is linear or branched which is unsubstituted or substituted with:

a) halogen, or

b) phenyl, which is unsubstituted or substituted with 1-5 substitutents independently selected from halogen, CN, OH, R¹³⁰², OR¹³⁰², NHSO₂R¹³⁰², N(C₁₋₆alkyl)SO₂R¹³⁰², SO₂R¹³⁰², SO₂NR¹³⁰⁵R¹³⁰⁶, NR¹³⁰⁵R¹³⁰⁶, CONR¹³⁰⁵R¹³⁰⁶, CO₂H, and CO₂C₁₋₆alkyl, wherein the C₁₋₆alkyl is linear or branched,

(4) phenyl which is unsubstituted or substituted with 1-5 substitutents independently selected from halogen, CN, OH, R¹³⁰², OR¹³⁰², NHSO₂R¹³⁰², N(C₁₋₆alkyl)SO₂R¹³⁰², SO₂R¹³⁰² SO₂NR¹³⁰⁵R¹³⁰⁶, NR³⁰⁵R³⁰⁶, CONR¹³⁰⁵R¹³⁰⁶, C₂H, and CO₂C₁₋₆alkyl, wherein the C₁₋₆alkyl is linear or branched,

(5) a 5- or 6-membered heterocyclic which may be saturated or unsaturated comprising 1-4 heteroatoms independently selected from N, S and O, the heterocycle being unsubstituted or substituted with 1-3 substituents independently selected from oxo, halogen, NO₂, CN, OH, R¹³⁰², OR¹³⁰², NHSO₂R¹³⁰², N(C₁₋₆alkyl)SO₂R¹³⁰², SO O₂R¹³²SO₂NR¹³⁰⁵R¹³⁰⁶, N¹³⁰⁵R¹³⁰⁶, CONR¹³⁰⁵R¹³⁰⁶ CO₂H, and CO₂C₁₋₆alkyl, wherein the C₁₋₆alkyl is linear or branched,

(6) C₃-6cycloalkyl, which is optionally substituted with 1-5 substituents independently selected from halogen, OH, C₁₋₆alkyl, and OC₁₋₆alkyl, wherein the C₁₋₆alkyl and OC₁₋₆ alkyl are linear or branched and optionally substituted with 1-5 halogens,

(7) OH,

(8) OR¹³⁰², and

(9) NR¹³⁰⁵R¹³⁰⁶;

R¹³⁰² is C₁₋₆alkyl, which is linear or branched and which is unsubstituted or substituted with 1-5 groups independently selected from halogen, CO₂H, and CO₂C₁₋₆alkyl, wherein the C₁₋₆alkyl is linear or branched;

R¹³⁰³, R¹³⁰⁴ and R¹³⁰⁷ are independently selected from the group consisting of:

(1) hydrogen,

(2) Cl-l₀alkyl, which is linear or branched and which is unsubstituted or substituted with one or more substituted selected from:

a) halogen,

b) hydroxy,

c) phenyl, which is unsubstituted or substituted with 1-5 substituted independently selected from halogen, OH, C₁₋₆alkyl, and OC₁₋₆alkyl, wherein the C₁₋₆alkyl is linear or branched and optionally substituted with 1-5 halogens,

d) naphthyl, wherein the naphthyl is optionally substituted with 1-5 substituents independently selected from halogen, OH, C₁₋₆alkyl, and OC₁₋₆alkyl, wherein the C₁₋₆alkyl is linear or branched and optionally substituted with 1-5 halogens,

e) CO₂H,

f) CO₂C₁₋₆alkyl,

g) CoNR¹³⁰⁵R³⁰⁶,

(3) CN,

(4) phenyl which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens,

(5) naphthyl which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens,

(6) CO₂H,

(7) CO₂C₁₋₆alkyl,

(8) CONR¹³⁰⁵R¹³⁰⁶, and

(9) C₃₋₆cycloalkyl, which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens;

R¹³⁰⁵ and R¹³⁰⁶ are independently selelcted from the group consisting of:

(1) hydrogen,

(2) phenyl, which is unsubstituted or substituted with substituents independently selected from halogen, OH, C₁₋₆alkyl, and OC₁₋₆alkyl, wherein the C₁₋₆alkyl is linear or branched and optionally substituted with 1-5 halogens,

(3) C₃₋₆cycloalkyl, which is unsubstituted or substituted with 1-5 substituents independently selected from C₁₋₆alkyl, and OC₁₋₆alkyl, wherein the C₁₋₆alkyl is linear or branched and optionally substituted with 1-5 halogens,

(4) C₁₋₆alkyl, which is linear or branched and which is unsubstituted or substituted with:

a) halogen, or

b) phenyl, which is unsubstituted or substituted with 1-5 substituents independently selected from halogen, OH, C₁₋₆alkyl, and OC₁₋₆alkyl, wherein the C₁₋₆alkyl is linear or branched and optionally substituted with 1-5 halogens,

or wherein R¹³⁰⁵ and R¹³⁰⁶ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆alkyl, and C₁₋₆alkoxy, wherein alkyl and alkoxy are unsubstituted with one to five halogens;

or wherein the group PM

has the formula XIV:

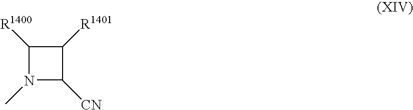

wherein R¹⁴⁰⁰ and R¹⁴⁰¹, independently of each other, are

a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R¹⁴⁰²), a boronic acid group (—B(OH)₂), a cyano group (—C≡N), a carboxylic acid group (—COOH), a carboxylic acid ester group (—COOR⁴⁰³), a carboxylic acid anhydride group (—CO—O—CO—R¹⁴⁰⁴), a hydroxamic acid group (—CO—NH(OH)), a N-substituted hydroxamic acid group (—CO—NR¹⁴⁰⁵(OH)), a O-substituted hydroxamic acid group (—CO—NH(OR¹⁴⁰⁶)), a carboxamide group (—CO—NH₂), a N-substituted or N,N-disubstituted carboxylic acid amide group, (—CO—NHR¹⁴⁰⁷; —CO—NR¹⁴⁰⁸, R¹⁴⁰⁹), an amido group (—HN—CO—R¹⁴¹⁰), a sulfonic acid group (—SO₃H), a sulfonamide group (—SO₂—NH₂), a N-substituted or N,N-disubstituted sulfonamide group (—SO₂—NHR¹⁴¹¹; —SO₂—NR¹⁴¹²R¹⁴¹³), an amidosulfone group (—NH—SO₂—R¹⁴¹⁴), a sulfone group (—SO₂—R¹⁴¹⁵), a phosphoric acid group (—OP(═O)(OH)₂), a phosphoric acid ester group (—OP(═O)(OR¹⁴¹⁶)(OR¹⁴¹⁷)), a phosphonic acid group (—P(═O)(OH)₂), an phosphonic acid ester group (—P(═O)(OR¹⁴¹⁸)(OR¹⁴¹⁹)), a halogen atom, a trifluormethyl group (—CF₃), a thiol group (—SH); a thioether group (—S—R¹⁴²⁰), a hydroxy group (—OH); an alkoxy group (—O—R¹⁴²¹), a tetrazole group, an amino group (—NH₂), or a N-substituted or N,N-disubstituted amino group (—NHR¹⁴²²; —NR¹⁴²³R¹⁴²⁴); and

which, independently of each other, can be substituted with one or more substituents, which can be the same or different; and,

wherein optionally, the pairs R¹⁴⁰⁸/R¹⁴⁰⁹, R¹⁴¹²/R¹⁴¹³, R¹⁴¹⁶/R¹⁴¹⁷, R¹⁴¹⁸/R¹⁴¹⁹ and R¹⁴²³/R¹⁴²⁴, independenly of each other, may form a part of a ring; and

wherein the substituents R¹⁴⁰², R¹⁴⁰³, R¹⁴⁰⁴, R¹⁴⁰⁵, R¹⁴⁰⁶, R¹⁴⁰⁷, R¹⁴⁰⁸, R¹⁴⁰⁹, R¹⁴¹⁰, R¹⁴¹¹ R¹⁴¹², R¹⁴¹³, R¹⁴¹⁴, R¹⁴¹⁵, R¹⁴¹⁶, R¹⁴¹⁷, R¹⁴¹⁸, R¹⁴¹⁹, R¹⁴²⁰, R¹⁴²¹, R¹⁴²², R¹⁴²³, and R¹⁴²⁴ independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula XV:

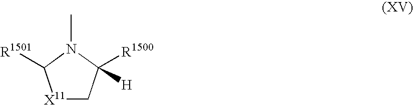

wherein X¹¹ is CH₂, CHF or CF₂;

wherein R¹⁵⁰⁰ is selected from the group consisting of alkylcarbonyl, arylcarbonyl, cyano, heterocyclecarbonyl, R¹⁵⁰²R¹⁵⁰³NC(O)—, B(OR¹⁵⁰⁴ o)₂, (1,2,3)-dioxoborolane and 4,4,5,5-tetramethyl(1,2,3)-dioxoborolane;

wherein R¹⁵⁰¹ is selected from the group consisting of alkoxyalkyl, alkyl, alkylcarbonyl, alkenyl, alkynyl, allenyl, arylalkyl, cycloalkyl, cycloalkylalkyl, cyano, haloalkyl, haloalkenyl, heterocyclealkyl, and hydroxyalkyl;

wherein R¹⁵⁰², R¹⁵⁰³ and R¹⁵⁰⁴ are each independently selected from the group consisting of hydrogen, alkyl, and arylalkyl;

with the proviso that the following compounds are excluded:

glutamin-thiazolidin (=Gln-Thia), glutamin-pyrrolidin (=Gln-Pyrr) (from WO 03/072556), glutamin-pyrrolidin-2-carboxylic acid (=Gln-Pro), glutamin-pyrrolidin-2-carboxamid (=Gln-Pro amid), and (S,S) 4-Amino-5-(2-cyano-2,5-dihydro-pyrrol-1-yl)-6-oxo-pentanoic acid amide (Gln-2-cyano-2,5-dihydro-pyrrolidin) (from WO 01/55105).

In a further embodiment, the present invention comprises a compound of the general formula (I)

NR¹R²—C(=EWG1)—(CR³R⁴)ₙ—CR⁵R⁶—CR⁷R⁸—CR⁹(NR¹⁰R¹¹)—C(=EWG2)-PM (I)

wherein n is 0 or 1;

wherein R¹R², R³, R⁴, R⁵, R⁶, R⁷, R⁸, R⁹, R¹⁰, and R¹¹ independently of each other are

a hydrogen atom; or

a substituted or unsubstituted alkyl group having 1 to 30 carbon atoms; or

a substituted or unsubstituted alkenyl group having 2 to 30 carbon atoms; or

a substituted or unsubstituted alkinyl group having 2 to 30 carbon atoms; or

a substituted or unsubstituted cycloalkyl group having 3 to 30 carbon atoms; or

a substituted or unsubstituted cycloalkenyl group having 3 to 30 carbon atoms;

or a substituted or unsubstituted cycloalkinyl group having 6 to 30 carbon atoms; or

a substituted or unsubstituted heteroalkyl group having 1 to 30 carbon atoms and 1 to 6 hetero atoms each independently selected from oxygen, nitrogen or sulfur; or

a substituted or unsubstituted heteroalkenyl group having 2 to 30 carbon atoms and 1 to 6 hetero atoms each independently selected from oxygen, nitrogen or sulfur; or

a substituted or unsubstituted heteroalkinyl group having 2 to 30 carbon atoms and 1 to 6 hetero atoms each independently selected from oxygen, nitrogen or sulfur; or

a substituted or unsubstituted heterocycloalkyl group having 1 to 30 carbon atoms, and 1 to 6 hetero atoms each independently selected from oxygen, nitrogen or sulfur; or

a substituted or unsubstituted heterocycloalkenyl group having 2 to 30 carbon atoms, and 1 to 6 hetero atoms each independently selected from oxygen, nitrogen or sulfur; or

a substituted or unsubstituted aryl group having 3 to 30 carbon atoms; or

a substituted or unsubstituted heteroaryl group having 1 to 30 carbon atoms, and 1 to 10 hetero atoms, each independently selected from oxygen, nitrogen or sulfur; or

a substituted or unsubstituted aryl-alkyl group having at least one substituted or unsubstituted aryl group each having 1 to 30 carbon atoms, and at least one substituted or unsubstituted alkyl group each having 1 to 30 carbon atoms; or

a substituted or unsubstituted heteroaryl-alkyl group having at least one substituted or unsubstituted heteroaryl group each having 1 to 30 carbon atoms, and 1 to 10 hetero atoms, each independently selected from oxygen, nitrogen or sulfur, and further, at least one substituted or unsubstituted alkyl group having having 1 to 30 carbon atoms; or

a substituted or unsubstituted aryl-heteroalkyl group having at least one substituted or unsubstituted aryl group each having 3 to 30 carbon atoms, and at least one substituted or unsubstituted heteroalkyl group each having 1 to 30 carbon atoms and 1 to 6 hetero atoms each independently selected from oxygen, nitrogen or sulfur; or

a substituted or unsubstituted heteroaryl-heteroalkyl group having at least one substituted or unsubstituted heteroaryl group each having 1 to 30 carbon atoms, and 1 to 10 hetero atoms, each independently selected from oxygen, nitrogen or sulfur, and further, at least one substituted or unsubstituted heteroalkyl group each having 1 to 30 carbon atoms and 1 to 6 hetero atoms each independently selected from oxygen, nitrogen or sulfur; or

a carbaldehyde (—CHO), a ketone group (—CO—R²⁰), a boronic acid group (—B(OH)₂), a cyano group (—C≡N), a carboxylic acid group (—COOH), a carboxylic acid ester group (—COOR²¹), a carboxylic acid anhydride group (—CO—O—CO—R²²), a hydroxamic acid group (—CO—NH(OH)), a N-substituted hydroxamic acid group (—CO—NR²³(OH)), a O-substituted hydroxamic acid group (—CO—NH(OR²⁴)), a carboxamide group (—CO—NH₂), a N-substituted or N,N-disubstituted carboxylic acid amide group, (—CO—NHR²⁵; —CO—NR²¹R²⁷), an amido group (—HN—CO—R²⁸), a sulfonic acid group (—SO₃H), a sulfonamide group (—SO₂—NH₂), a N-substituted or N,N-disubstituted sulfonamide group (—SO₂—NHR²⁹-SO₂—NR³⁰R³¹), an amidosulfone group (—NH—SO₂—R³²), a sulfone group (—SO₂—R³³), a phosphoric acid group (—OP(═O)(OH)₂), a phosphoric acid ester group (—OP(═O)(OR³⁴)(OR³⁵)), a phosphonic acid group (—P(═O)(OH)₂), an phosphonic acid ester group (—P(═O)(OR³⁶)(OR³⁷)), a halogen atom, a trifluormethyl group (—CF₃), a thiol group (—SH); a thioether group (—S—R³⁸), a hydroxy group (—OH); an alkoxy group (—O—R³⁹), a tetrazole group, an amino group (—NH₂), or a N-substituted or N,N-disubstituted amino group (—NHR⁴⁰; —NRR⁴²);

which each independently can be substituted with one or more substituents, which can be the same or different; and,

wherein optionally, any two of the groups R¹, R², R³, R⁴, R⁵, R⁶, R⁷, R⁸, R⁹, R¹⁰, and R¹¹, as well the pairs R²⁶/R²⁷, R³⁰/R³, R³⁴/R³⁵, R³⁶/R³⁷ and R⁴¹/R⁴², independenly of each other, may form a part of a ring; and

wherein the substituent, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, and R⁴² independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group.

In a further embodiment, the present invention comprises a compound of the general formula (I)

NR¹R²—C(=EWG1)—(CR³R⁴)ₙ—CR⁵R⁶—CR⁷R⁸—CR⁹(NR¹⁰R¹¹)—C(=EWG2)—PM (I)

wherein n is 0 or 1;

wherein R¹, R², R³, R⁴, R⁵, R⁶, R⁷, R⁸, R⁹, R¹⁰, and R¹¹ independently of each other are

a hydrogen atom; or

a substituted or unsubstituted alkyl group having 1 to 20 carbon atoms; or

a substituted or unsubstituted alkenyl group having 2 to 20 carbon atoms; or

a substituted or unsubstituted alkinyl group having 2 to 20 carbon atoms; or

a substituted or unsubstituted cycloalkyl group having 3 to 20 carbon atoms; or

a substituted or unsubstituted cycloalkenyl group having 3 to 20 carbon atoms;

or a substituted or unsubstituted cycloalkinyl group having 6 to 20 carbon atoms; or

a substituted or unsubstituted heteroalkyl group having 1 to 20 carbon atoms and 1 to 3 hetero atoms each independently selected from oxygen, nitrogen or sulfur; or

a substituted or unsubstituted heteroalkenyl group having 2 to 20 carbon atoms and 1 to 3 hetero atoms each independently selected from oxygen, nitrogen or sulfur; or

a substituted or unsubstituted heteroalkinyl group having 2 to 20 carbon atoms and 1 to 3 hetero atoms each independently selected from oxygen, nitrogen or sulfur; or

a substituted or unsubstituted heterocycloalkyl group having 1 to 20 carbon atoms, and 1 to 3 hetero atoms each independently selected from oxygen, nitrogen or sulfur; or

a substituted or unsubstituted heterocycloalkenyl group having 2 to 20 carbon atoms, and 1 to 3 hetero atoms each independently selected from oxygen, nitrogen or sulfur; or

a substituted or unsubstituted aryl group having 3 to 20 carbon atoms; or

a substituted or unsubstituted heteroaryl group having 1 to 20 carbon atoms, and 1 to 4 hetero atoms, each independently selected from oxygen, nitrogen or sulfur; or

a substituted or unsubstituted aryl-alkyl group having at least one substituted or unsubstituted aryl group each having 1 to 20 carbon atoms, and at least one substituted or unsubstituted alkyl group each having 1 to 20 carbon atoms; or

a substituted or unsubstituted heteroaryl-alkyl group having at least one substituted or unsubstituted heteroaryl group each having 1 to 20 carbon atoms, and 1 to 4 hetero atoms, each independently selected from oxygen, nitrogen or sulfur, and further, at least one substituted or unsubstituted alkyl group having having 1 to 20 carbon atoms; or

a substituted or unsubstituted aryl-heteroalkyl group having at least one substituted or unsubstituted aryl group each having 3 to 20 carbon atoms, and at least one substituted or unsubstituted heteroalkyl group each having 1 to 20 carbon atoms and 1 to 3 hetero atoms each independently selected from oxygen, nitrogen or sulfur; or

a substituted or unsubstituted heteroaryl-heteroalkyl group having at least one substituted or unsubstituted heteroaryl group each having 1 to 20 carbon atoms, and 1 to 4 hetero atoms, each independently selected from oxygen, nitrogen or sulfur, and further, at least one substituted or unsubstituted heteroalkyl group each having 1 to 20 carbon atoms and 1 to 4 hetero atoms each independently selected from oxygen, nitrogen or sulfur; or

a carbaldehyde (—CHO), a ketone group (—CO—R²⁰), a boronic acid group (—B(OH)₂), a cyano group (—C_N), a carboxylic acid group (—COOH), a carboxylic acid ester group (—COOR²¹), a carboxylic acid anhydride group (—CO—O—CO—R²²), a hydroxamic acid group (—CO—NH(OH)), a N-substituted hydroxamic acid group (—CO—NR²³(OH)), a O-substituted hydroxamic acid group (—CO—NH(OR²⁴)), a carboxamide group (—CO—NH₂), a N-substituted or N,N-disubstituted carboxylic acid amide group, (—CO—NHR²⁵; —CO—NR²⁶R²⁷), an amido group (—HN—CO—R²⁸), a sulfonic acid group (—SO₃H), a sulfonamide group (—SO₂—NH₂), a N-substituted or N,N-disubstituted sulfonamide group (—SO₂—NHR²⁹; —SO₂—NR³¹R³¹), an amidosulfone group (—NH—SO₂—R³²), a sulfone group (—SO₂—R³³), a phosphoric acid group (—OP(═O)(OH)₂), a phosphoric acid ester group (—OP(═O)(OR³⁴)(OR³⁵)), a phosphonic acid group (—P(═O)(OH)₂), an phosphonic acid ester group (—P(═O)(OR³⁶)(OR³⁷)), a halogen atom, a trifluormethyl group (—CF₃), a thiol group (—SH); a thioether group (—S—R³⁸), a hydroxy group (—OH); an alkoxy group (—O—R³⁹), a tetrazole group, an amino group (—NH₂), or a N-substituted or N,N-disubstituted amino group (—NHR⁴⁰; —NR⁴R⁴²);

which each independently can be substituted with one or more substituents, which can be the same or different; and,

wherein optionally, any two of the groups R¹, R², R³, R⁴, R⁵, R⁶, R⁷, R⁸, R⁹, R¹⁰, and R¹¹, as well the pairs R²⁶/R²⁷, R³⁰/R³¹, R³⁴/R³⁵, R³⁶/R³⁷ and R⁴¹/R⁴², independenly of each other, may form a part of a ring; and

wherein the substituents R²⁰, R²¹, R²², R²³, R²⁴, R²⁵, R²⁶, R²⁷, R²⁸, R²⁹, R³⁰, R³¹, R³², R³³, R³⁴R³⁵, R³⁶, R³⁷, R³⁸, R³⁹, R⁴⁰, R⁴¹, and R⁴² independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group.

In a preferred embodiment, the present invention comprises a compound of the general formula (I)

NR¹R²—C(=EWG1)-(CR³, R⁴)ₙ—CR⁵R⁶—CR⁷R⁸—CR⁹(NR¹⁰OR¹¹)—C(=EWG2)-PM (I)

wherein n is 0 or 1;

wherein R¹, R², R³, R⁴, R⁵, R⁶, R⁷, R⁸, R⁹, R¹⁰, and R¹¹ independently of each other are

a hydrogen atom; or

a straight or branched chain, substituted or unsubstituted alkyl group comprising methyl (—CH₃) and ethyl (—C₂H₅); or

a halogen comprising a fluoro, chloro, bromo or iodo atom; or

a cyano group; a thiol group; a hydroxy group; a carboxyl group, a tetrazole group, an amino group; an amido group;

and wherein EWG1 and EWG2 is a double bound oxygen (═O).

In a more preferred embodiment, the present invention comprises a compound of the general formula (I)

wherein n is 0;

wherein R¹, R², R³, R⁴, R⁵, R⁶, R⁷, R⁸, R⁹, R¹⁰, and R¹¹, is each a hydrogen atom; and

wherein EWG1 and EWG2 is a double bound oxygen (═O).

In a further more preferred embodiment, the present invention comprises a compound of the general formula (I)

wherein n is 1;

wherein R¹, R², R³, R⁴, R⁵, R⁶, R⁷, R⁸, R⁹, R¹⁰, and R¹¹ is each a hydrogen atom; and

wherein EWG1 and EWG2 is a double bound oxygen (═O).

Preferred are compounds as disclosed above

wherein the group PM

has the formula (II)

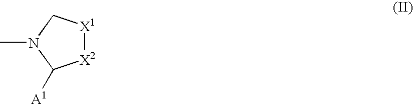

wherein X¹ is CR⁵¹R⁵², O, S, or NR⁵³; and wherein X² is CR⁵⁴R⁵⁵, O, S, or NR⁵⁶; and

wherein R⁵¹, R⁵², R⁵³, R⁵⁴, R⁵⁵, and R⁵⁶, independently of each other, are

a hydrogen atom (—H); or an C₁, C₂, C₃, C₄, C₅, C₆, C₇, C₈ and C₉branched or straight chain alkyl, C₂, C₃, C₄, C₅, C₆, C₇, C₈ and C₉branched or straight chain alkenyl, C₂, C₃, C₄, C₅, C₆, C₇, C₈ and C₉branched or straight chain alkinyl, C₃, C₄, C₅, C₆, C₇, C₈ and C₉cycloalkyl, C₅, C₆, C₇, CS and C₉cycloalkenyl, aryl, heteroaryl or amino (—NH₂), or a N-substituted or N,N-disubstituted amino group (—NHR⁸⁰; —NR⁸¹R⁸²); and

which, independently of each other, can be substituted with one or more substituents, which can be the same or different; and,

wherein optionally, any two of the groups R⁵¹, R⁵², R⁵³, R⁵⁴, R⁵⁵, and R⁵⁶, if present, as well as the pairs R⁶⁶/R⁶⁷R⁷⁰/R⁷¹, R⁷⁴/R⁷⁵, R⁷⁶/R⁷⁷ and R⁸¹/R⁸², independently of each other, may form a part of a ring; and

wherein the substituents R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³ R⁷⁴, R⁷⁵, R⁷⁶, R⁷⁷, R⁷⁸, R⁷⁹, R⁸⁰, R⁸¹, and R⁸², independently of each other, are a hydrogen atom (—H), or a C₁, C₂, C₃, C₄, C₅, C₆, C₇, C₈ and C₉branched or straight chain alkyl, aryl, heteroaryl, amino, halo, carbonyl, C₁, C₂, C₃, C₄, C₅, C₆, C₇, C₈ and C₉branched or straight chain alkoxy, C₂, C₃, C₄, C₅, C₆, C₇, C₈ and Cg branched or straight chain alkenoxy, phenyloxy, benzyloxy, C₃, C₄, C₅, C₆, C₇, C₈ and C₉ cycloalkyl, cyano, amido, thiol trifluoromethyl, or hydroxy group; and

wherein A¹ is

a hydrogen atom (—H) or a carbaldehyde (—CHO), a ketone group (—CO—R¹⁰⁰), a boronic acid group (—B(OH)₂), a cyano group (—C≡N), a carboxylic acid group (—COOH), a carboxylic acid ester group (—COOR¹⁰¹), a carboxylic acid anhydride group (—CO—O—CO—R¹⁰²), a hydroxamic acid group (—CO—NH(OH)), a N-substituted hydroxamic acid group (—CO—NR¹⁰³(OH)), a O-substituted hydroxamic acid group (—CO—NH(OR¹⁰⁴)), a carboxamide group (—CO—NH₂), a N-substituted or N,N-disubstituted carboxylic acid amide group, (—CO—NHR¹⁰⁵; —CO—NR¹⁰⁶R¹⁰⁷), an amido group (—HN—CO—R¹⁰⁸), a sulfonic acid group (—SO₃H), a sulfonamide group (—SO₂—NH₂), a N-substituted or N,N-disubstituted sulfonamide group (—SO₂—NHR¹⁰⁹; —SO₂—NR¹¹⁰R¹¹¹), an amidosulfone group (—NH—SO₂—R¹¹²), a sulfone group (—SO₂—R¹¹³), a phosphoric acid group (—OP(═O)(OH)₂), a phosphoric acid ester group (—OP(═O)(OR¹¹⁴)(OR¹¹⁵)), a phosphonic acid group (—P(═O)(OH)₂), an phosphonic acid ester group (—P(═O)(OR¹¹⁶)(OR¹¹⁷)), a halogen atom, a trifluormethyl group (—CF₃), a thiol group (—SH); a thioether group (—S—R¹¹⁸), a hydroxy group (—OH); an alkoxy group (—O—R¹¹⁹), a tetrazole group, an amino group (—NH₂), or a N-substituted or N,N-disubstituted amino group (—NHR¹²⁰; —NR¹²¹R¹²²); and

which, independently of each other, can be substituted with one or more substituents, which can be the same or different; and,

wherein optionally, the pairs R¹⁰⁶/R¹⁰⁷, R¹¹⁰I/R¹¹¹, R¹¹⁴/R¹¹⁵, R¹¹⁶/R¹¹⁷ and R¹²¹/R¹²², independently of each other, may form a part of a ring; and

wherein the substituents R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹, R^{112, R}¹¹³, R¹¹⁴, R¹¹⁵, R¹¹⁶, R¹¹⁷, R¹¹⁸, R¹¹⁹, R¹²⁰, R¹²¹, and R¹²², independently of each other, are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (IfI)

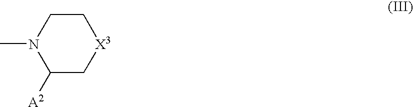

wherein X³ is CR¹³¹, R¹³², O. S, or NR¹³³; and

wherein R¹³¹, R¹³², and R¹³³, independently of each other, are

a hydrogen atom (—H); or an C₁, C₂, C₃, C₄, C₅, C₆, C₇, C₈ and C₉branched or straight chain alkyl, C₂, C₃, C₄, C₅, C₆, C₇, C₈ and Cg branched or straight chain alkenyl, C₂, C₃, C₄, C₅, C₆, C₇, C₈ and C₉branched or straight chain alkinyl, C₃, C₄, C₅, C₆, C₇, C₈ and C₉ cycloalkyl, C₅, C₆, C₇, C₈ and Cg cycloalkenyl, aryl, heteroaryl or an amino group (—NH₂), or a N-substituted or N,N-disubstituted amino group (—NHR⁶⁰; —NR⁶¹R⁶²); and

which, independently of each other, can be substituted with one or more substituents, which can be the same or different; and,

wherein optionally, the the pair R¹³¹/R¹³², if present, as well the pairs R¹⁴⁶/R¹⁴⁷, R¹⁵⁰/R¹⁵¹, R¹⁵⁴/R¹⁵⁵, R¹⁵⁶/R¹⁵⁷ and R¹⁶¹/R¹⁶², independenly of each other, may form a part of a ring; and

wherein the substituents R¹⁴⁰, R¹⁴¹, R¹⁴², R¹⁴³, R¹⁴⁴, R¹⁴⁵, R¹⁴⁶, R¹⁴⁷, R¹⁴⁸, R¹⁴⁹, R¹⁵⁰R¹⁵¹, R¹⁵², R¹⁵³, R¹⁵⁴, R¹⁵⁵, R¹⁵⁶, R¹⁵⁷, R¹⁵⁸, R¹⁵⁹, R¹⁶⁰, R¹⁶¹, and R¹⁶², independently of each other are a hydrogen atom (—H), or a C₁, C₂, C₃, C₄, Cs, C₆, C₇, C₈ and C₉branched or straight chain alkyl, aryl, heteroaryl, amino, halo, carbonyl, C₁, C₂, C₃, C₄, C₅, C₆, C₇, C₈ and Cg branched or straight chain alkoxy, C₂, C₃, C₄, C₅, C₆, C₇, C₈ and C₉ branched or straight chain alkenoxy, phenyloxy, benzyloxy, C₃, C₄, C₅, C₆, C₇, C₈ and C₉cycloalkyl, cyano, amido, thiol, trifluoromethyl, or hydroxy group; and

wherein A² is

a hydrogen atom (—H); or a carbaldehyde (—CHO), a ketone group (—CO—R¹⁸⁰), a boronic acid group (—B(OH)₂), a cyano group (—C≡N), a carboxylic acid group (—COOH), a carboxylic acid ester group (—COOR¹⁸¹), a carboxylic acid anhydride group (—CO—O—CO—R¹⁸²), a hydroxamic acid group (—CO—NH(OH)), a N-substituted hydroxamic acid group (—CO—NR¹⁸³(OH)), a O-substituted hydroxamic acid group (—CO—NH(OR¹⁸⁴)), a carboxamide group (—CO—NH₂), a N-substituted or N,N-disubstituted carboxylic acid amide group, (—CO—NHR¹⁸⁵; —CO—NR¹⁸⁶, R¹⁸⁷), an amido group (—HN—CO—R¹⁸⁸), a sulfonic acid group (—SO₃H), a sulfonamide group (—SO₂—NH₂), a N-substituted or N,N-disubstituted sulfonamide group (—SO₂—NHR¹⁸⁹; —SO₂—NR¹⁹⁰R¹⁹¹), an amidosulfone group (—NH—SO₂—R¹⁹²), a suffone group (—SO₂—R¹⁹³), a phosphoric acid group (—OP(═O)(OH)₂), a phosphoric acid ester group (—OP(═O)(OR¹⁹⁴)(OR¹⁹⁵)), a phosphonic acid group (—P(═O)(OH)₂), an phosphonic acid ester group (—P(═O)(OR¹⁹⁶)(OR¹⁹⁷)), a halogen atom, a trifluormethyl group (—CF₃), a thiol group (—SH); a thioether group (—S—R¹⁹⁸), a hydroxy group (—OH); an alkoxy group (—O—R¹⁹⁹), a tetrazole group, an amino group (—NH₂), or a N-substituted or N,N-disubstituted amino group (—NHR²⁰⁰; —NR²⁰¹R²⁰²); and

which, independently of each other, can be substituted with one or more substituents, which can be the same or different; and,

wherein optionally, the pairs R¹⁸⁶/R¹⁸⁷, R¹⁹⁰/R¹⁹¹, R⁹⁴/R¹⁹⁵, R⁶/R¹⁹⁷ and R²⁰¹/R²⁰² independenly of each other, may form a part of a ring; and

wherein the substituents R¹⁸⁰, R¹⁸¹, R¹⁸², R¹⁸³, R¹⁸⁴, R¹⁸⁵, R¹⁸⁶, R¹⁸⁷, R¹⁸⁸, R¹⁸⁹, R¹⁹⁰ R¹⁹¹, R¹⁹², R¹⁹³, R¹⁹⁴, R¹⁹⁵, R¹⁹⁶, R¹⁹⁷, R¹⁹⁸, R¹⁹⁹, R²⁰⁰, R²⁰¹ and R²⁰², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (IV)

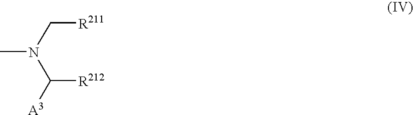

wherein R²¹¹ and R²¹², independently of each other, are

a hydrogen atom (—H); or an C₁, C₂, C₃, C₄, C₅, C₆, C₇, C₈ and Cg branched or straight chain alkyl, C₂, C₃, C₄, C₅, C₆, C₇, C₈ and Cs branched or straight chain alkenyl, C₂, C₃, C₄, C₅, C₆, C₇, C₈ and Cg branched or straight chain alkinyl, C₃, C₄, C₅, C₆, C₇, C₈ and Cg cycloalkyl, C₅, C₆, C₇, C₈ and Cs cycloalkenyl, aryl, heteroaryl or an amino group (—NH₂), or a N-substituted or N,N-disubstituted amino group (—NHR²⁴⁰; —NR²⁴¹R²⁴²); and

which, independently of each other, can be substituted with one or more substituents, which can be the same or different; and,

wherein optionally, the pairs R²²⁶/R²²⁷, R²³⁰/R²³¹, R²³⁴/R²³⁵, R²³⁶, R²³⁷ and R²⁴¹/R²⁴² independenly of each other, may form a part of a ring; and

wherein the substituents R²²⁰, R²²¹, R²²², R²²³, R²²⁴, R²²⁵, R²²⁶, R²²⁷, R²²⁸, R²²⁹, R²³⁰R²³¹, R²³², R²³³, R²³⁴, R²³⁵, R²³⁶, R²³⁷, R²³, R²³⁹, R²⁴⁰, R²⁴¹, and R²⁴², independently of each other, are a hydrogen atom (—H), or a C₁, C₂, C₃, C₄, C₅, C₆, C₇, C₈ and Cg branched or straight chain alkyl, aryl, heteroaryl, amino, halo, carbonyl, C₁, C₂, C₃, C₄, C₅, C₆, C₇, C₈ and Cg branched or straight chain alkoxy, C₂, C₃, C₄, C₅, C₆, C₇, C₈ and Cg branched or straight chain alkenoxy, phenyloxy, benzyloxy, C₃, C₄, C₅, C₆, C₇, C₈ and Cg cycloalkyl, cyano, amido, thiol, trifluoromethyl, or hydroxy group; and

wherein A³ is

a hydrogen atom (—H); or a carbaldehyde (—CHO), a ketone group (—CO—R²⁶⁰), a boronic acid group (—B(OH)₂), a cyano group (—C≡N), a carboxylic acid group (—COOH), a carboxylic acid ester group (—COOR²⁶¹), a carboxylic acid anhydride group (—CO—O—CO—R²⁶²), a hydroxamic acid group (—CO—NH(OH)), a N-substituted hydroxamic acid group (—CO—NR²⁶ (OH)), a O-substituted hydroxamic acid group (—CO—NH(OR²⁶⁴)), a carboxamide group (—CO—NH₂), a N-substituted or N,N-disubstituted carboxylic acid amide group, (—CO—NHR²⁶⁵; —CO—NR²⁶⁶, R²⁶⁷), an amido group (—HN—CO—R²⁶⁸), a sulfonic acid group (—SO₃H), a sulfonamide group (—SO₂—NH₂), a N-substituted or N,N-disubstituted sulfonamide group (—SO₂—NHR²⁶⁹; —SO₂—NR²⁷⁰R²⁷¹), an amidosulfone group (—NH—SO₂—R²⁷²), a sulfone group (—SO₂—R²⁷³), a phosphoric acid group (—OP(═O)(OH)₂), a phosphoric acid ester group (—OP(═O)(OR²⁷⁴)(OR²⁷⁵)), a phosphonic acid group (—P(═O)(OH)₂), an phosphonic acid ester group (—P(═O)(OR²⁷⁶)(OR²⁷⁷)), a halogen atom, a trifluormethyl group (—CF₃), a thiol group (—SH); a thioether group (—S—R²⁷⁸), a hydroxy group (—OH); an alkoxy group (—O—R²⁷⁹), a tetrazole group, an amino group (—NH₂), or a N-substituted or N,N-disubstituted amino group (—NHR²⁸⁰; —NR²⁸¹R²⁸²); and

which, independently of each other, can be substituted with one or more substituents, which can be the same or different; and,

wherein optionally, the pairs R²⁶⁶/R²⁶⁷, R²⁷⁰/R²⁷¹, R²⁷⁴/R²⁷⁵, R²⁷⁶/R²⁷⁷ and R²⁸¹/R²⁸² independenly of each other, may form a part of a ring; and

wherein the substituents R²⁶⁰, R²⁶¹, R²⁶², R²⁶³, R²⁶⁴, R²⁶⁵, R²⁶⁶, R²⁶⁷, R²⁶⁸, R²⁶⁹, R²⁷⁰R²⁷¹, R²⁷², R²⁷³, R²⁷⁴, R²⁷⁵, R²⁷⁶, R²⁷⁷, R²⁷⁸, R²⁷⁹, R²⁸⁰, R²⁸¹, and R²⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (V)

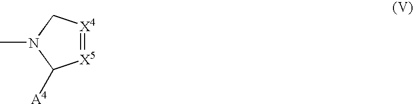

wherein X⁴ is CR²⁹¹ or N; and

wherein X⁵ is CR²⁹² or N; and

wherein R²⁹¹ and R²⁹², independently of each other, are

a hydrogen atom (—H); or an C₁, C₂, C₃, C₄, C₅, C₆, C₇, C₈ and C₉branched or straight chain alkyl, C₂, C₃, C₄, C₅, C₆, C₇, C₈ and C₉branched or straight chain alkenyl, C₂, C₃, C₄, C₅, C₆, C₇, C₈ and C₉branched or straight chain alkinyl, C₃, C₄, C₅, C₆, C₇, C₈ and C₉ cycloalkyl, C₅, C₆, C₇, C₈ and Cg cycloalkenyl, aryl, heteroaryl group, or an amino group (—NH₂), or a N-substituted or N,N-disubstituted aniino group (—NHR³²⁰; —NR³²¹R³²²); and

which, independently of each other, can be substituted with one or more substituents, which can be the same or different; and,

wherein optionally, the the pair R²⁹¹/R²⁹², if present, as well the pairs R³⁰⁶/R³⁰⁷, R³¹⁰/R³¹¹, R³¹⁴/R³¹⁵, R³¹⁶/R³¹⁷ and R³²¹/R³²², independenly of each other, may form a part of a ring; and

wherein the substituents R³⁰⁰, R³⁰¹, R³⁰², R³⁰³, R³⁰⁴, R³⁰⁵, R³⁰⁶, R³⁰⁷, R³⁰⁸, R³⁰⁹, R^{310 R}³¹¹, R³¹², R³¹³, R³, R³¹⁴, R³¹⁵, R³¹⁶, R³¹⁷, R³¹⁸, R³¹⁹, R³²⁰, R³²¹, and R³²², independently of each other are a hydrogen atom (—H), or a C₁, C₂, C₃, C₄, C₅, C₆, C₇, C₈ and Cg branched or straight chain alkyl, aryl, heteroaryl, amino, halo, carbonyl, C₁, C₂, C₃, C₄, C₅, C₆, C₇, C₈ and Cs branched or straight chain alkoxy, C₂, C₃, C₄, C₅, C₆, C₇, C₈ and Cg branched or straight chain alkenoxy, phenyloxy, benzyloxy, C₃, C₄, C₅, C₆, C₇, C₈ and Cg cycloalkyl, cyano, amido, thiol, trifluoromethyl, or hydroxy group; and

wherein A⁴ is

a hydrogen atom (—H); or a carbaldehyde (—CHO), a ketone group (—CO—R³⁴⁰), a boronic acid group (—B(OH)₂), a cyano group (—C≡N), a carboxylic acid group (—COOH), a carboxylic acid ester group (—COOR³⁴¹), a carboxylic acid anhydride group (—CO—O—CO—R³⁴²), a hydroxamic acid group (—CO—NH(OH)), a N-substituted hydroxamic acid group (—CO—NR³⁴³ (OH)), a O-substituted hydroxamic acid group (—CO—NH(OR³⁴⁴)), a carboxamide group (—CO—NH₂), a N-substituted or N,N-disubstituted carboxylic acid aamide group, (—CO—NHR³⁴⁵; —CO—NR³⁴⁶R³⁴⁷), an amido group (—HN—CO—R³⁴⁸), a sulfonic acid group (—SO₃H), a sulfonamide group (—SO₂—NH₂), a N-substituted or N,N-disubstituted sulfonamide group (—SO₂—NHR³⁴⁹; —SO₂—NR³⁵° R³⁵¹), an amidosulfone group (—NH—SO₂—R³²), a sulfone group (—SO₂—R³⁵³), a phosphoric acid group (—OP(═O)(OH)₂), a phosphoric acid ester group (—OP(═O)(OR³⁵⁴)(OR³⁵⁵)), a phosphonic acid group (—P(═O)(OH)₂), an phosphonic acid ester group (—P(═O)(OR³⁵⁶)(OR³⁵⁷)), a halogen atom, a trifluormethyl group (—CF₃), a thiol group (—SH); a thioether group (—S—R³⁵⁸), a hydroxy group (—OH); an alkoxy group (—O—R³⁵⁹), a tetrazole group, an amino group (—NH₂), or a N-substituted or N,N-disubstituted amino group (—NHR³⁶⁰; —NR³⁶R³⁶²); and

which, independently of each other, can be substituted with one or more substituents, which can be the same or different; and,

wherein optionally, the pairs R³⁴⁶/R³⁴⁷, R³⁵⁰/R³⁵¹, R³⁵⁴/R³⁵⁵, R³⁵⁶/R³⁵⁷ and R³⁶¹/R³⁶², independenly of each other, may form a part of a ring; and

wherein the substituents R³⁴⁰, R³⁴¹, R³⁴², R³⁴³, R³⁴⁴, R³⁴⁵, R³⁴⁶, R³⁴⁷, R³⁴⁸, R³⁴⁹, R³⁵⁰ R³⁵¹, R³⁵², R³⁵³, R³⁵⁴, R³⁵⁵, R³⁵⁶, R³⁵⁷, R³⁵⁸, R³⁵⁹, R³⁶⁰, R³⁶¹, and R³⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (VI)

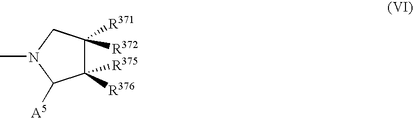

wherein R³⁷¹, R³⁷², R³⁷⁵ and R³⁷⁶ independently of each other, a hydrogen atom (—H); or a C₁, C₂, C₃, C₄, C₅, C₆, C₇, C₈ and Cg branched or straight chain alkyl, C₂, C₃, C₄, C₅, C₆, C₇, C₈ and Cg branched or straight chain alkenyl, C₂, C₃, C₄, C₅, C₆, C₇, C₈ and C₉ branched or straight chain alkinyl, C₃, C₄, C₅, C₆, C₇, C₈ and Cg cycloalkyl, C₅, C₆, C₇, C₈ and C₉ cycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R³⁸⁰), a boronic acid group (—B(OH)₂), a cyano group (—C≡N), a carboxylic acid group (—COOH), a carboxylic acid ester group (—COOR³⁸¹), a halogen atom, a trifluormethyl group (—CF₃), a thiol group (—SH); a thioether group (—S—R³⁹⁸), a hydroxy group (—OH); an alkoxy group (—O—R³⁹⁹), a tetrazole group, an amino group (—NH₂), or a N-substituted or N,N-disubstituted amino group (—NHR⁴⁰⁰; —NR⁴⁰¹R⁴⁰²); and

which, independently of each other, can be substituted with one or more substituents, which can be the same or different; and,

wherein optionally, any two of the groups R³⁷¹, R³⁷², R³⁷⁵, and R³⁷⁶, as well as the pairs R^{386/387}, R³⁹⁰/R³⁹¹, R³⁹⁴/R³⁹⁵, R³⁹⁶/R³⁹⁷, R⁴⁰¹/R⁴⁰², independenly of each other, may form a part of a ring; and

wherein the substituents R³⁸⁰, R³⁸¹, R³⁸², R³⁸³, R³⁸⁴, R³⁸⁵, R³⁸⁶, R³⁸⁷, R³⁸⁸, R³⁸⁹, R³⁹⁰, R³⁹¹, R³⁹², R³⁹³, R³⁹⁴, R³⁹⁵, R³⁹⁶, R³⁹⁷, R³⁹⁸, R³⁹⁹, R⁴⁰⁰, R⁴⁰¹, and R⁴⁰², independently each other are a hydrogen atom (—H), or a C₁, C₂, C₃, C₄, Cs, C₆, C₇, C₈ and C₉ branched or straight chain alkyl, aryl, heteroaryl, amino, halo, carbonyl, C₁, C₂, C₃, C₄, Cs, C₆, C₇, C₈ and Cg branched or straight chain alkoxy, C₂, C₃, C₄, Cs, C₆, C₇, C₈ and C₉branched or straight chain alkenoxy, phenyloxy, benzyloxy, C₃, C₄, C₅, C₆, C₇, C₈ and C₉cycloalkyl, cyano, amido, thiol, trifluoromethyl, or hydroxy group; and

alternatively; the two groups R³⁷¹ and R³⁷² can be together an oxo (═O) or hydroxyimino (═N—OH) group; and

alternatively; the two groups R³⁷⁵ and R³⁷⁶ can be together an oxo (═O) or hydroxyimino (═N—OH) group; and

wherein A⁵ is

a hydrogen atom (—H); or a carbaldehyde (—CHO), a ketone group (—CO—R⁴²⁰), a boronic acid group (—B(OH)₂), a cyano group (—C≡N), a carboxylic acid group (—COOH), a carboxylic acid ester group (—COOR⁴²¹), a carboxylic acid anhydride group (—CO—O—CO—R⁴²²), a hydroxamic acid group (—CO—NH(OH)), a N-substituted hydroxamic acid group (—CO—NR¹²¹(OH)), a O-substituted hydroxamic acid group (—CO—NH(OR⁴²)), a carboxamide group (—CO—NH₂), a N-substituted or N,N-disubstituted carboxylic acid amide group, (—CO—NHR⁴²⁵; —CO—NR⁴²⁶R⁴²⁷), an amido group (—HN—CO—R⁴²⁸), a sulfonic acid group (—SO₃H), a sulfonamide group (—SO₂—NH₂), a N-substituted or N,N-disubstituted sulfonamide group (—SO₂—NHR¹²¹; —SO₂—NR³⁰R¹³), an amidosulfone group (—NH—SO₂—R⁴³²), a sulfone group (—SO₂—R⁴³³), a phosphoric acid group (—OP(═O)(OH)₂), a phosphoric acid ester group (—OP(═O)(OR⁴³⁴)(OR⁴³⁵)), a phosphonic acid group (—P(═O)(OH)₂), an phosphonic acid ester group (—P(═O)(OR⁴³⁶)(OR⁴³⁷)), a halogen atom, a trifluormethyl group (—CF₃), a thiol group (—SH); a thioether group (—S—R³⁸), a hydroxy group (—OH); an alkoxy group (—O—R⁴³⁹), a tetrazole group, an amino group (—NH₂), or a N-substituted or N,N-disubstituted amino group (—NHR⁴⁴⁰; —NR⁴⁴¹R⁴⁴²); and

which, independently of each other, can be substituted with one or more substituents, which can be the same or different; and,

wherein optionally, the pairs R⁴²⁶/R⁴²⁷, R⁴³⁰/R⁴³¹R⁴³⁴/R⁴³⁵, R⁴³⁶/R⁴³⁷ and R⁴⁴¹/R⁴⁴², independenly of each other, may form a part of a ring; and

wherein the substituents R⁴²⁰, R⁴²¹, R⁴²², R⁴²³, R⁴²⁴, R⁴²⁵, R⁴²⁶, R⁴²⁷, R⁴²⁸, R⁴²⁹, R⁴³⁰, R⁴³¹, R⁴³², R⁴³³, R⁴³⁴, R⁴³⁵, R⁴³⁶, R⁴³⁷, R⁴³⁸, R⁴³⁹, R⁴⁴⁰, R⁴⁴¹, and R⁴⁴², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (VII)

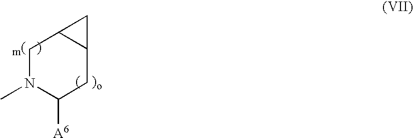

wherein m is equal to 1 or 2, and o is equal to 1 or 2, and m or o can be 0;

wherein A⁶ is a hydrogen atom (—H); or a carbaldehyde (—CHO), a ketone group (—CO—R⁴⁶⁰), a boronic acid group (—B(OH)₂), a cyano group (—C≡N), a carboxylic acid group (—COOH), a carboxylic acid ester group (—COOR⁴⁶¹), a carboxylic acid anhydride group (—CO—O—CO—R⁴⁶²), a hydroxamic acid group (—CO—NH(OH)), a N-substituted hydroxamic acid group (—CO—NR⁴⁶³(OH)), a O-substituted hydroxamic acid group (—CO—NH(OR⁴⁶⁴)), a carboxamide group (—CO—NH₂), a N-substituted or N,N-disubstituted carboxylic acid amide group, (—CO—NHR⁴⁶⁵; —CO—NR⁴⁶⁶R⁴⁶⁷), an amido group (—HN—CO—R⁴⁶⁸), a sulfonic acid group (—SO₃H), a sulfonamide group (—SO₂—NH₂), a N-substituted or N,N-disubstituted sulfonamide group (—SO₂—NHR⁴⁶⁹; —SO₂—NR⁴⁷⁰R⁴⁷¹), an amidosulfone group (—NH—SO₂—R⁴⁷²), a sulfone group (—SO₂—R⁴⁷³), a phosphoric acid group (—OP(═O)(OH)₂), a phosphoric acid ester group (—OP(═O)(OR⁴⁷⁴)(OR⁴⁷⁵)), a phosphonic acid group (—P(═O)(OH)₂), an phosphonic acid ester group (—P(═O)(OR⁴⁷⁶)(OR⁴⁷⁷)), a halogen atom, a trifluormethyl group (—CF₃), a thiol group (—SH); a thioether group (—S—R⁴⁷⁸), a hydroxy group (—OH); an alkoxy group (—O—R⁴⁷⁹), a tetrazole group, an amino group (—NH₂), or a N-substituted or N,N-disubstituted amino group (—NHR⁴⁸⁰; —NR⁴⁸¹R⁴⁸²);

which, independently of each other, can be substituted with one or more substituents, which can be the same or different; and,

wherein optionally, the pairs R⁴⁶⁶/R⁴⁶⁷, R⁴⁷⁰/R⁴⁷¹, R⁴⁷⁴/R⁴⁷⁵, R⁴⁷⁶/R⁴⁷⁷ and R⁴⁸¹/R⁴⁸² independenly of each other, may form a part of a ring; and

wherein the substituents R⁴⁶⁰, R⁴⁶¹, R⁴⁶², R⁴⁶³, R⁴⁶⁴, R⁴⁶⁵, R⁴⁶⁶, R⁴⁶⁷, R⁴⁶⁸, R⁴⁶⁹, R⁴⁷⁰ R⁴⁷¹, R⁴⁷², R⁴⁷³, R⁴⁷⁴, R⁴⁷⁵, R⁴⁷⁶, R⁴⁷⁷, R⁴⁷⁸, R⁴⁷⁹, R⁴⁸⁰, R⁴⁸¹, and R⁴⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (VIII)

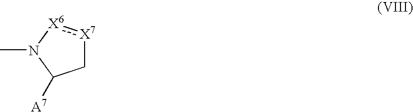

wherein X⁶ is selected from CR⁴⁹⁰R⁴⁹¹, O, S or NR⁴⁹², when the bond between X⁶ and X⁷ is a single bond; and

wherein X⁷ is selected from CR⁴⁹³R⁴⁹⁴, O, S, or NR⁴⁹⁵, when the bond between X⁶ and X⁷ is a single bond;

or alternatively,

wherein X⁶ is selected from CR⁴⁹⁶ or N, when the bond between X⁶ and X⁷ is a double bond; and

wherein X⁷ is selected from CR⁴⁹⁷ or N, when the bond between X⁶ and X⁷ is a double bond; and

wherein R⁴⁹⁰, R⁴⁹¹, R⁴⁹², R⁴⁹³, R⁴⁹⁴, R⁴⁹⁵, R⁴⁹⁶, and R⁴⁹⁷, independently of each other, are a hydrogen atom (—H); or a C₁, C₂, C₃, C₄, C₅, C₆, C₇, C₈ and Cg branched or straight chain alkyl, C₂, C₃, C₄, C₅, C₆, C₇, C₈ and Cg branched or straight chain alkenyl, C₂, C₃, C₄, C₅, C₆, C₇, C₈ and Cs branched or straight chain alkinyl, C₃, C₄, C₅, C₆, C₇, C₈ and Cg cycloalkyl, C₅, C₆, C₇, C₈ and Cg cycloalkenyl, heteroalkyl, aryl, heteroaryl, aryl-alkyl, heteroaryl-alkyl, aryl-heteroalkyl, heteroaryl-heteroalkyl group or, a tetrazole group, an amino group (—NH₂), or a N-substituted or N,N-disubstituted amino group (_NIHR⁵²⁰; —NR⁵²¹, R⁵²²); and

which, independently of each other, can be substituted with one or more substituents, which can be the same or different; and,

wherein optionally, any two the groups R⁴⁹⁰, R⁴⁹¹, R⁴⁹², R⁴⁹³, R⁴⁹⁴, R₄₉₅ R⁴⁹⁶ and R⁴⁹⁷ if present, as well as the pairs R⁵⁰⁶/R⁵⁰⁷, R⁵¹⁰/R⁵¹¹, R⁵¹⁴/R⁵¹⁵, R⁵¹⁶/R⁵¹⁷ and R⁵²¹/R⁵²², independenly of each other, may form a part of a ring; and

wherein the substituents R⁵⁰⁰, R⁵⁰¹, R⁵⁰², R⁵⁰³, R⁵⁰⁴, R⁵⁰⁵, R⁵⁰⁶, R⁵⁰⁷, R⁵⁰⁸, R⁵⁰⁹, R⁵¹⁰, R⁵¹¹, R⁵¹², R⁵¹³, R⁵¹⁴, R⁵¹⁵, R⁵¹⁶, R⁵¹⁷, R⁵¹⁸, R⁵¹⁹, R⁵²⁰, R⁵²¹, and R⁵²², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group; and

wherein A⁷ is

a hydrogen atom (—H); or a carbaldehyde (—CHO), a ketone group (—CO—R⁵⁴⁰), a boronic acid group (—B(OH)₂), a cyano group (—C≡N), a carboxylic acid group (—COOH), a carboxylic acid ester group (—COOR⁵⁴¹), a carboxylic acid anhydride group (—CO—O—CO—R⁵⁴²), a hydroxamic acid group (—CO—NH(OH)), a N-substituted hydroxamic acid group (—CO—NR⁵⁴³(OH)), a O-substituted hydroxamic acid group (—CO—NH(OR⁵⁴⁴)), a carboxamide group (—CO—NH₂), a N-substituted or N,N-disubstituted carboxylic acid amide group, (—CO—NHR⁵⁴⁵; —CO—NR⁵⁴⁶, R⁵⁴⁷), an amido group (—HN—CO—R⁵⁴⁸), a sulfonic acid group (—SO₃H), a sulfonamide group (—SO₂—NH₂), a N-substituted or N,N-disubstituted sulfonamide group (—SO₂—NHR⁵⁴⁹; —SO₂—NR⁵⁵⁰R⁵⁵¹), an amidosulfone group (—NH—SO₂—R⁵⁵²), a sulfone group (—SO₂—R⁵⁵³), a phosphoric acid group (—OP(═O)(OH)₂), a phosphoric acid ester group (—OP(═O)(OR⁵⁵⁴)(OR⁵⁵⁵)), a phosphonic acid group (—P(═O)(OH)₂), an phosphonic acid ester group (—P(═O)(OR⁵⁵⁶)(OR⁵⁵⁷)), a halogen atom, a trifluormethyl group (—CF₃), a thiol group (—SH); a thioether group (—S—R⁵⁵⁸), a hydroxy group (—OH); an alkoxy group (—O—R⁵⁵⁹), a tetrazole group, an amino group (—NH₂), or a N-substituted or N,N-disubstituted amino group (—NHR⁵⁶⁰; —NR⁵⁶¹R⁵⁶²); and

which, independently of each other, can be substituted with one or more substituents, which can be the same or different; and,

wherein optionally, the pairs R⁵⁴⁶/R⁵⁴⁷, R⁵⁵⁰/R⁵⁵¹, R⁵⁵⁴/R⁵⁵⁵, R⁵⁵⁶/R⁵⁵⁷ and R⁵⁶¹/R⁵⁶² independenly of each other, may form a part of a ring; and

wherein the substituents R⁵⁴⁰, R⁵⁴¹, R⁵⁴², R⁵⁴³, R⁵⁴⁴, R⁵⁴⁵, R⁵⁴⁶, R⁵⁴⁷, R⁵⁴⁸, R⁵⁴⁹, R⁵⁵⁰, R⁵⁵¹, R⁵⁵², R⁵⁵³, R⁵⁵⁴, R⁵⁵⁵, R⁵⁵⁶, R⁵⁵⁷, R⁵⁵⁸, R⁵⁵⁹, R⁵⁶⁰, R⁵⁶, and R⁵⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (IX) or (IXa)

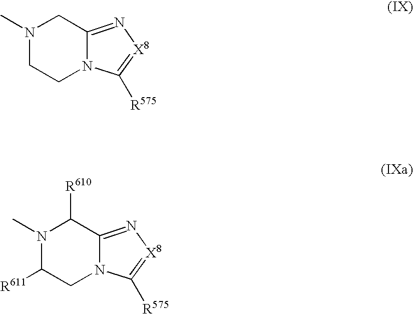

wherein X⁸ is N or CR⁵⁷⁰; and

wherein R⁵⁷⁰, R⁵⁷⁵, R⁶¹⁰ and R⁶¹¹ independently of each other, are a hydrogen atom (—H); or an C₁, C₂, C₃, C₄, C₅, C₆, C₇, C₈ and Cg branched or straight chain alkyl, C₂, C₃, C₄, C₅, C₆, C₇, C₈ and Cg branched or straight chain alkenyl, C₂, C₃, C₄, C₅, C₆, C₇, C₈ and Cg branched or straight chain alkinyl, C₃, C₄, C₅, C₆, C₇, C₈ and Cg cycloalkyl, C₅, C₆, C₇, C₈ and Cg cycloalkenyl, aryl, heteroaryl, aryl-alkyl, aryl-heteroalkyl group or, a carbaldehyde (—CHO), a ketone group (—CO—R⁵⁸⁰), a boronic acid group (—B(OH)₂), a cyano group (—C≡N), a carboxylic acid group (—COOH), a carboxylic acid ester group (—COOR⁵⁸¹), a carboxylic acid anhydride group (—CO—O—CO—R⁵⁸²), a hydroxamic acid group (—CO—NH(OH)), a N-substituted hydroxamic acid group (—CO—NR⁵⁸³(OH)), a O-substituted hydroxamic acid group (—CO—NH(OR⁵⁸⁴)), a carboxamide group (—CO—NH₂), a N-substituted or N,N-disubstituted carboxylic acid amide group, (—CO—NHR⁵⁸⁵; —CO—NR⁵⁸⁶R⁵⁸⁷), an amido group (—HN—CO—R⁵⁸⁸), a sulfonic acid group (—SO₃H), a sulfonamide group (—SO₂—NH₂), a N-substituted or N,N-disubstituted sulfonamide group (—SO₂—NHR⁵⁸⁹; —SO₂—NR⁵⁹⁰R⁵⁹¹), an amidosulfone group (—NH—SO₂—R⁵⁹²), a sulfone group (—SO₂—R⁵⁹³), a phosphoric acid group (—OP(═O)(OH)₂), a phosphoric acid ester group (—OP(═O)(OR⁵⁹⁴)(OR⁵⁹⁵)), a phosphonic acid group (—P(═O)(OH)₂), an phosphonic acid ester group (—P(═O)(OR⁵⁹⁶)(OR⁹⁷)), a halogen atom, a trifluormethyl group (—CF₃), a thiol group (—SH); a thioether group (—S—R⁵⁹⁸), a hydroxy group (—OH); an alkoxy group (—O—R⁵⁹⁹), a tetrazole group, an amino group (—NH₂), or a N-substituted or N,N-disubstituted amino group (—NHR⁶⁰⁰; —NR⁶⁰⁰R⁶⁰²);

which, independently of each other, can be substituted with one or more substituents, which can be the same or different; and,

wherein optionally, the pairs R⁵⁷⁰/R⁵⁷⁵, if present, as well as the pairs R⁵⁸⁶/R⁵⁸⁷, R⁵⁹⁰/R⁵⁹¹, R⁵⁹⁴/R⁵⁹⁵, R⁵⁹⁶/R⁵⁹⁷ and R⁶⁰¹/R⁶⁰², independenly of each other, may form a part of a ring; and

wherein the substituents R⁵⁸⁰, R⁵⁸¹, R⁵⁸², R⁵⁸³, R⁵⁸⁴, R⁵⁸⁵, R⁵⁸⁶, R⁵⁸⁷, R⁵⁸⁸, R⁵⁸⁹, R⁵⁹⁰ R⁵⁹¹, R⁵⁹², R⁵⁹³, R⁵⁹⁴, R⁵⁹⁵, R⁵⁹⁶, R⁵⁹⁷, R⁵⁹⁸, R⁵⁹⁹, R⁶⁰⁰, R⁶⁰¹, and R⁶⁰², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (X)

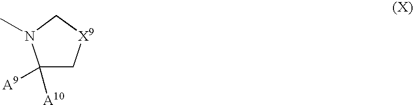

wherein the groups X⁹ is CR⁹⁰⁰R⁹⁰¹, S, SO, SO₂or NR⁹⁰²

wherein R⁹⁰⁰, R⁹⁰¹ and R⁹⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR⁹¹⁰R⁹¹¹.

wherein A⁹ and A¹⁰ are, independently of each other, selected from hydrogen, cyano, —C(═O)NR⁹¹²R⁹¹³, or C₁, C₂, C₃, C₄, C₅or C₆alkyl, which is linear or branched and is optionally substituted with 1, 2, 3, 4, or 5 halogens;

wherein

R⁹¹⁰ and R⁹¹², are, independently of each other, selected from hydrogen, or C₁, C₂, C₃, C₄, C₅or C₆alkyl, which is linear or branched and is optionally substituted with 1, 2, 3, 4, or 5 halogens; and

R⁹¹¹ and R⁹¹³, are, independently of each other, selected from the group consisting of

(1) phenyl, which is optionally substituted with 1, 2, 3, 4, or 5, substituents independently selected from halogen and R⁹²⁰;

(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of

(a) hydroxy,

(b) —COOH,

(c) —COO(C₁, C₂, C₃, C₄, C₅or C₆alkyl), i.e. ester,

(d) phenyl,

(e) naphthyl,

(f) C₃, C₄, C₅or C₆cycloalkyl,

(g) a 5- or 6 membered heterocycle which may be saturated or unsaturated comprising 1, 2, 3, or 4 heteroatoms independently selected from nitrogen, oxygen or sulfur;

(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;

wherein said C₃, C₄, C₅or C₆cycloalkyl, phenyl, naphthyl, are optionally substituted with 1, 2, 3, 4, or 5 substituents independently selected from halogen and R⁹²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R⁹²⁰; and

(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;

wherein R⁹²⁰ is selected from the group consisting of:

(1) hydroxy;

(2) cyano;

(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;

(4) C₁, C₂, C₃, C₄, C₅, C₆, C₇, C₈, Cg or C₁₀alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from

(a) hydroxy;

(b) —COOH;

(c) —COO(C₁, C₂, C₃, C₄, C₅or C₆alkyl) i.e. ester, which may linear or branched and is optionally substituted with 1, 2, 3, 4, or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR⁹²⁵, R⁹²⁵;

(g) —SO₂NR⁹²⁵R⁹²⁵;

(h) —NR⁹²⁵—C(═O)R⁹²⁵

(i) —NR⁹²⁵—C(═O)NR⁹²⁵R⁹²⁵

(j) —NR⁹²⁵COOR⁹³⁰

(k) —O—CO—R⁹³⁰

(l) —O—CO—NR⁹²⁵R⁹²⁵;

(m) —NR⁹²⁵SO R⁹³⁰;

(n) _NR⁹²⁵R⁹²⁵;

(o) phenyl which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(5) OC₁, OC₂, OC₃, OC₄, OC₅, OC₆, OC₇, OC₈, OC₉or OC₁₀alkyl, which is linear or branched and is optionally substituted with 0, 1, 2, 3, 4, or 5 halogen atoms and 0, 1, or 2 substitutents selected from

(a) hydroxy;

(b) —COOH;

(c) —COO(C₁, C₂, C₃, C₄, C₅or C₆alkyl) i.e. ester, which may be linear or branched and is optionally substituted with 1, 2, 3, 4 or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, Cs or C₆alkyl, and —OC₁, —OC₂, —OC₃, —OC₄, —OC₅or —OC₆alkyl, said C₁, C₂, C₃, C₄, C₅or C₆alkyl, and —OC I, —OC₂, —OC₃, —OC₄, —OC₅or —OC₆alkyl being linear or branched and optionally substituted with 1, 2, 3, 4, or 5 halogens;

(f) —CONR¹²⁵R⁹²⁵;

(g) —SO₂NR⁹²⁵R⁹²⁵;

(h) —NR⁹²⁵—C(═O)R⁹²⁵

(i) —NR⁹²⁵C(═O)NRR⁹²⁵R⁹²⁵

(j) —NR⁹²⁵ COOR⁹³⁰

(k) —O—CO—R⁹³⁰

(l) —O—CO—NR⁹²⁵R⁹²⁵;

(m) —NR⁹²⁵SO₂R⁹³⁰;

(n) NR⁹²⁵, R⁹²⁵;

(o) phenyl, which is optionally substituted with 1, 2, 3, 4, or 5 groups independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(6) —COOH;

(7) —COO(C₁, C₂, C₃, C₄, C₅or C₆alkyl) i.e. ester, which may be linear or branched and is optionally substituted with 1, 2, 3, 4, 5 halogens;

(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.

(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(10)—CONR⁹²⁵R⁹²⁵;

(11) —SO₂NR⁹²⁵R⁹²⁵;

(12) —NR²¹—C(═O)R⁹²¹

(13)—NR⁹²⁵—C(═O)NR⁹²⁵R⁹²⁵;

(14) NR⁹²⁵ COOR⁹³⁰ (15)—O—CO—R⁹³⁰

(16)—O—CO—NR⁹²⁵, R⁹²⁵;

(17) NR⁹²⁵SO₂R⁹³⁰;

(18) —NR⁹²⁵, R⁹²⁵;

(19) phenyl, which is optionally substituted with 1, 2, 3, 4, or 5 group independently selected from halogen, hydroxy, C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(Cl, C₂, C₃, C₄, C₅or C₆alkyl) i.e. ester being linear or branched and optionally substituted with 1, 2, 3, 4, or 5 halogens;

wherein R⁹³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.

wherein R⁹²⁵ is selected from R⁹³⁰ and hydrogen.

wherein the group PM

has the formula (XI)

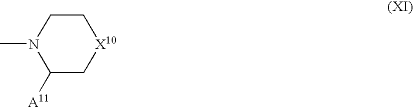

wherein the groups X¹⁰ is CR¹⁰⁰⁰R¹⁰⁰¹, S. SO, SO₂or NR¹⁰⁰²

wherein R¹⁰⁰⁰, R¹⁰⁰¹, and R¹⁰⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR¹⁰¹⁰R¹⁰¹¹.

and A¹¹ is selected from

hydrogen, cyano, —C(═O)NR¹⁰¹²R¹⁰¹³, or C₁, C₂, C₃, C₄, C₅or C₆alkyl, which is linear or branched and is optionally substituted with 1, 2, 3, 4, or 5 halogens;

wherein

R¹⁰¹⁰ and R¹⁰¹², are, independently of each other, selected from hydrogen, or C₁, C₂, C₃, C₄, C₅or C₆alkyl, which is linear or branched and is optionally substituted with 1, 2, 3, 4, or 5 halogens; and

R¹⁰¹¹ and R¹⁰¹³, are, independently of each other, selected from the group consisting of

(1) phenyl, which is optionally substituted with 1, 2, 3, 4, or 5, substituents independently selected from halogen and R¹⁰²⁰;

(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of

(a) hydroxy,

(b) —COOH,

(c) —COO(C₁, C₂, C₃, C₄, C₅or C₆alkyl), i.e. ester,

(d) phenyl,

(e) naphthyl,

(f) C₃, C₄, C₅or C₆cycloalkyl,

(g) a 5- or 6 membered htereocycle which may be saturated or unsaturated comprising 1, 2, 3, or 4 heteroatoms independently selected from nitrogen, oxygen or sulfur;

(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;

wherein said C₃, C₄, C₅or C₆cycloalkyl, phenyl, naphthyl, are optionally substituted with 1, 2, 3, 4, or 5 substituents independently selected from halogen and R¹⁰²⁰, and said 5 or 6 membered heterocycle and said 8, 9 or 10-membered 5 bicyclic ring system are each optionally substituted with 1, 2, 3, 4, or 5 substituents independently selected from from oxo, hodroxy, halogen, and R¹⁰²⁰; and

(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;

wherein R¹⁰²⁰ is selected from the group consisting of:

(1) hydroxy;

(2) cyano;

(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;

(4) C₁, C₂, C₃, C₄, C₅, C₆, C₇, C₈, Cg or C₁₀alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from

(a) hydroxy;

(b) —COOH;

(c) —COO(C₁, C₂, C₃, C₄, Cs or C₆alkyl) i.e. ester, which may linear or branched and is optionally substituted with 1, 2, 3, 4, or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR¹⁰²⁵R¹⁰²⁵;

(g) —SO₂NR¹⁰²⁵R¹⁰²⁵;

(h) —NR¹⁰²⁵—C(═O)R¹⁰²⁵

(i) —NR¹⁰²⁵—C(═O)NR¹⁰²⁵R¹⁰²⁵

(j) —NRo25 COOR¹⁰³⁰

(k) —O—CO—R¹⁰³⁰

(l) —O—CO—NR¹⁰²⁵R¹⁰²⁵;

(m) —NR¹⁰²⁵SO₂R¹⁰³⁰;

(n) —NR¹⁰²⁵R¹⁰²⁵;

(o) phenyl which is optionally substituted with 1, 2, 3, 4, or 5 group independently selected from halogen, hydroxy, C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(5) OC₁, OC₂, OC₃, OC₄, OC₅, OC₆, OC₇, OC₈, OC₉or OC₁₀alkyl, which is linear or branched and is optionally substituted with 0, 1, 2, 3, 4, or 5 halogen atoms and 0, 1, or 2 substitutents selected from

(a) hydroxy;

(b) —COOH;

(c) —COO(C₁, C₂, C₃, C₄, C₅or C₆alkyl) i.e. ester, which may be linear or branched and is optionally substituted with 1, 2, 3, 4 or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, Cs or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR¹⁰²⁵, R¹⁰²⁵;

(g) —SO₂NR¹⁰²⁵R¹⁰²⁵

(h) —NR¹⁰²⁵—C(═O)R¹⁰²⁵

(i) —NR¹⁰²⁵—C(═O)NR¹⁰²⁵, R¹⁰²⁵

(j) —NR¹⁰²⁵COOR¹⁰³⁰

(k) —O—CO—R¹⁰³⁰

(l) —O—CO—NR¹⁰²⁵R¹⁰²⁵;

(m) —NR¹⁰²⁵SO₂R¹⁰³⁰;

(n) _NR¹⁰²⁵, R¹⁰²⁵;

(o) phenyl, which is optionally substituted with 1, 2, 3, 4, or 5 groups independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(6) —COOH;

(7) —COO(C₁, C₂, C₃, C₄, C₅or C₆alkyl) i.e. ester, which may be linear or branched and is optionally substituted with 1, 2, 3, 4, 5 halogens;

(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.

(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(10)—CONR¹⁰²⁵R¹⁰²⁵;

(11) —SO₂NR¹⁰²⁵R¹⁰²⁵;

(12) —NR¹⁰²⁵—C(═O)R¹⁰²⁵

(13)—NR¹⁰²⁵—C(═O)NR¹²⁵R¹⁰²⁵

(14) —NR⁹²⁵COOR¹⁰³⁰

(15)—O—CO—R¹⁰³⁰

(16)—O—CO—NR¹⁰²⁵R¹⁰²⁵;

(17) NRSO¹⁰²⁵SO₂R¹⁰³⁰;

(18)—NR¹⁰²⁵, R¹⁰²⁵;

(19) phenyl, which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;

wherein R¹⁰³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.

wherein R¹⁰²⁵ is selected from R¹⁰³⁰ and hydrogen.

or wherein the group PM

has the formula (XII)

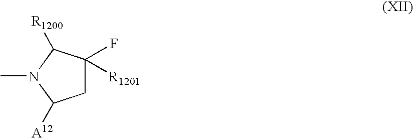

wherein the groups R¹²⁰¹ is hydrogen or fluoro.

wherein R¹²⁰⁰ und A¹² is selected from hydrogen and cyano, and the other is hydrogen.

or wherein the group PM

has the formula XIII:

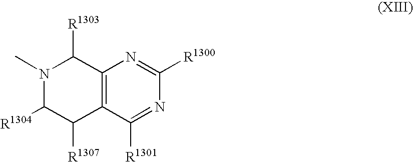

wherein:

R¹³⁰⁰ and R¹³⁰¹ are independently selected from the group consisting of:

(1) hydrogen,

(2) CN,

(3) C₁₋₁₀alkyl, which is linear or branched which is unsubstituted or substituted with:

a) halogen, or

b) phenyl, which is unsubstituted or substituted with 1-5 substitutents independently selected from halogen, CN, OH, R¹³⁰², OR¹³⁰², NHSO₂R¹³⁰², N(C₁₋₆alkyl)SO₂R¹³⁰², SO₂R¹³⁰², SO₂NR¹³⁰⁵R¹³⁰⁶, NR¹³⁰⁵R¹³⁰⁶, CONR¹³⁰⁵R¹³⁰⁶, CO₂H, and CO₂C₁₋₆alkyl, wherein the C₁₋₆alkyl is linear or branched,

(4) phenyl which is unsubstituted or substituted with 1-5 substitutents independently selected from halogen, CN, OH, R¹³⁰², OR¹³⁰², NHSO₂R¹³⁰² N(C₁₋₆alkyl)SO₂R¹³⁰², SO₂R¹³⁰², SO₂NR¹³⁰⁵R¹³⁰⁶ NR¹³⁰⁵R¹³⁰⁶ CONR¹³⁰⁵R¹³⁰⁶, CO₂H, and CO₂C₁₋₆alkyl, wherein the C₁₋₆alkyl is linear or branched,

(5) a 5- or 6-membered heterocyclic which may be saturated or unsaturated comprising 1-4 heteroatoms independently selected from N, S and O, the heterocycle being unsubstituted or substituted with 1-3 substituents independently selected from oxo, halogen, NO₂, CN, OH, R^{1302, OR}¹³⁰², NHSO₂R¹³⁰², N(C₁₋₆alkyl)SO₂R¹³⁰⁶, SO₂R¹³⁰², SO₂NR¹³⁰⁵R¹³⁰⁶, NR¹³⁰⁵R¹³⁰⁶, CONR¹³⁰⁵R¹³⁰⁶, CO₂H, and CO₂C₁₋₆alkyl, wherein the C₁₋₆alkyl is linear or branched,

(6) C₃₋₆cycloalkyl, which is optionally substituted with 1-5 substituents independently selected from halogen, OH, C₁₋₆alkyl, and OC₁₋₆alkyl, wherein the C₁₋₆alkyl and OC₁₋₆ alkyl are linear or branched and optionally substituted with 1-5 halogens,

(7) OH,

(8) OR³⁰², and

(9) NR¹³⁰⁵R¹³⁰⁶

R¹⁰² is C₁₋₆alkyl, which is linear or branched and which is unsubstituted or substituted with 1-5 groups independently selected from halogen, CO₂H, and CO2C₁₋₆alkyl, wherein the C₁₋₆alkyl is linear or branched;

R¹³⁰³ is selected from the group consisting of:

(1) hydrogen,

(2) C₁₋₁₀alkyl, which is linear or branched and which is unsubstituted or substituted with one or more substituted selected from:

a) halogen,

b) hydroxy,

c) phenyl, which is unsubstituted or substituted with 1-5 substitutents independently selected from halogen, OH, C₁₋₆alkyl, and OC₁₋₆alkyl, wherein the C₁₋₆alkyl is linear or branched and optionally substituted with 1-5 halogens,

d) naphthyl, wherein the naphthyl is optionally substituted with 1-5 substituents independently selected from halogen, OH, C₁₋₆alkyl, and OC₁₋₆alkyl, wherein the C₁₋₆alkyl is linear or branched and optionally substituted with 1-5 halogens,

h) CO₂H,

i) CO₂C₁₋₆alkyl,

j) CoNR¹³⁰⁵R¹³⁰⁶,

(3) CN,

(4) phenyl which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens (5) naphthyl which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens,

(6) CO₂H,

(7) CO₂C₁₋₆alkyl,

(8) CONR¹³⁰⁵R¹³⁰⁶, and

(9) C₃-6cycloalkyl, which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens

R¹³⁰⁵ and R¹³⁰⁶ are independently selected from the group consisting of:

(1) hydrogen,

(2) phenyl, which is unsubstituted or substituted with substituents independently selected from halogen, OH, C₁₋₆alkyl, and OC₁₋₆alkyl, wherein the C₁₋₆alkyl is linear or branched and optionally substituted with 1-5 halogens,

(3) C₃₋₆cycloalkyl, which is unsubstituted or substituted with 1-5 substituents independently selected from C₁₋₆alkyl, and OC₁₋₆alkyl, wherein the C₁₋₆alkyl is linear or branched and optionally substituted with 1-5 halogens,

(4) C₁₋₆alkyl, which is linear or branched and which is unsubstituted or substituted with:

a) halogen, or

b) phenyl, which is unsubstituted or substituted with 1-5 substituents independently selected from halogen, OH, C₁₋₆alkyl, and OC₁₋₆alkyl, wherein the C₁₋₆alkyl is linear or branched and optionally substituted with 1-5 halogens,

or wherein R¹³⁰⁵ and R¹³⁰⁶ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆alkyl, and C₁₋₆alkoxy, wherein alkyl and alkoxy are unsubstituted with one to five halogens;

R¹³⁰⁴ and R¹³⁰⁷ are hydrogen;

or wherein the group PM

has the formula XIV:

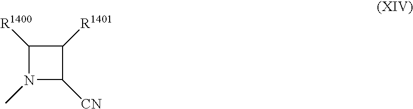

wherein R¹⁴⁰⁰ and R¹⁴⁰¹, independently of each other, are a hydrogen atom (—H); or halogen, cyano or ethynyl;

or wherein the group PM

has the formula XV:

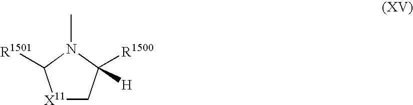

wherein X¹ is CH₂, CHF or CF²;

wherein R¹⁵⁰⁰ is cyano;

wherein R¹⁵⁰¹ is selected from the group consisting of alkoxyalkyl, alkyl, alkylcarbonyl, alkenyl, alkynyl, allenyl, arylalkyl, cycloalkyl, cycloalkylalkyl, cyano, haloalkyl, haloalkenyl, heterocyclealkyl, and hydroxyalkyl;

Preferred are compounds as disclosed above

wherein the group PM

has the formula (II)

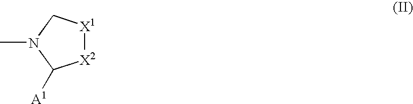

wherein X¹ is CR⁵¹R⁵², O, S, or NR⁵³; and

wherein X² is CR⁵⁴R⁵⁵, O, S, or NR⁵⁶; and

wherein R⁵¹, R⁵², R⁵³, R⁵⁴, R⁵⁵, and R⁵⁶, independently of each other, are

a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group or, an amino group (—NH₂), or a N-substituted or N,N-disubstituted amino group (—NHR⁸⁰; —NR⁸¹R⁸²); and

which, independently of each other, can be substituted with one or more substituents, which can be the same or different; and,

wherein optionally, any two of the groups R⁵¹, R⁵², R⁵³, R⁵⁴, R⁵⁵, and R⁵⁶, if present, as well as the pairs R⁶⁶/R⁶⁷, R⁷⁰/R⁷¹, R⁷⁴/R⁷⁵, R⁷⁶/R⁷⁷ and R⁸/R⁸², independently of each other, may form a part of a ring; and

wherein the substituents R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, and R⁸², independently of each other, are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅ branched or straight chain alkenoxy, phenyloxy, benzyloxy, C₃, C₄, C₅ cycloalkyl, cyano, amido, thiol trifluoromethyl, or hydroxy group; and

wherein A¹ is

a hydrogen atom (—H) or a carbaldehyde (—CHO), a ketone group (—CO—R¹⁰o), a boronic acid group (—B(OH)₂), a cyano group (—C≡N), a carboxylic acid group (—COOH), a carboxylic acid ester group (—COOR¹⁰¹), a carboxylic acid anhydride group (—CO—O—CO—R¹⁰²), a hydroxamic acid group (—CO—NH(OH)), a N-substituted hydroxamic acid group (—CO—NR¹⁰³(OH)), a O-substituted hydroxamic acid group (—CO—NH(OR¹⁰⁴)), a carboxamide group (—CO—NH₂), a N-substituted or N,N-disubstituted carboxylic acid amide group, (—CO—NHR¹⁰⁵; —CO—NR¹⁰⁶R¹⁰⁷), an amido group (—HN—CO—R¹⁰⁸), a sulfonic acid group (—SO₃H), a sulfonamide group (—SO₂—NH₂), a N-substituted or N,N-disubstituted sulfonamide group (—SO₂—NHR¹⁰⁹; —SO₂—NR¹¹⁰R¹¹¹), an amidosulfone group (—NH—SO₂—R¹¹²), a sulfone group (—SO₂—R¹¹³), a phosphoric acid group (—OP(═O)(OH)₂), a phosphoric acid ester group (—OP(═O)(OR¹¹⁴)(OR¹¹⁵)), a phosphonic acid group (—P(═O)(OH)₂), an phosphonic acid ester group (—P(═O)(OR¹¹⁶)(OR¹¹⁷)), a halogen atom, a trifluormethyl group (—CF₃), a thiol group (—SH); a thioether group (—S—R¹¹⁸), a hydroxy group (—OH); an alkoxy group (—O—R¹¹⁹), a tetrazole group, an amino group (—NH₂), or a N-substituted or N,N-disubstituted amino group (—NHR¹²⁰; —NR¹²¹R¹²²); and wherein optionally, the pairs R¹⁰⁶/R¹⁰⁷, R¹¹⁰/R¹¹¹, R¹¹⁴/R¹¹⁵, R¹¹⁶/R¹¹⁷ and R¹²¹/R¹²², independently of each other, may form a part of a ring; and

which, independently of each other, can be substituted with one or more substituents, which can be the same or different; and,

wherein the substituents R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹, R¹¹², R¹¹³, R¹¹⁴, R¹¹⁵, R¹¹⁶, R¹¹⁷, R¹¹⁸, R¹¹⁹, R¹²⁰, R¹²¹, and R¹²², independently of each other, are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (III)

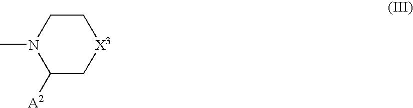

wherein X³ is CR¹³¹R¹³², O, S, or NR¹³³; and

wherein R¹³¹, R¹³², and R¹³³, independently of each other, are

a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, Cs, C₆, and C₇cycloalkyl, aryl, heteroaryl group or, an amino group (—NH₂), or a N-substituted or N,N-disubstituted amino group (—NHR¹⁶⁰; —NR¹⁶¹R¹⁶²); and

which, independently of each other, can be substituted with one or more substituents, which can be the same or different; and,

wherein optionally, the the pair R¹³¹/R¹³², if present, as well the pairs R¹⁴⁶/R¹⁴⁷, R¹⁵⁰/R¹⁵¹, R¹⁵⁴/R¹⁵⁵, R¹⁵⁶/R¹⁵⁷ and R¹⁶¹/R¹⁶², independenly of each other, may form a part of a ring; and

wherein the substituents R¹⁴⁰, R¹⁴¹, R¹⁴², R¹⁴³, R¹⁴⁴, R¹⁴⁵, R¹⁴⁶, R¹⁴⁷, R¹⁴⁸, R¹⁴⁹, R¹⁵⁰R¹⁵¹, R¹⁵², R¹⁵³, R¹⁵⁴, R¹⁵⁵, R¹⁵⁶, R¹⁵⁷, R¹⁵⁸, R¹⁵⁹, R¹⁶⁰, R¹⁶¹, and R¹⁶², independently of each other are a hydrogen atom (—H), or a C₁, C₂, C₃, C₄, and Cs branched or straight chain alkyl, aryl, heteroaryl, amino, halo, carbonyl, C₁, C₂, C₃, C₄, Cs, branched or straight chain alkoxy, C₂, C₃, C₄, Cs branched or straight chain alkenoxy, phenyloxy, benzyloxy, C₃, C₄, Cs cycloalkyl, cyano, amido, thiol trifluoromethyl, or hydroxy group; and

wherein A² is

a hydrogen atom (—H); or a carbaldehyde (—CHO), a ketone group (—CO—R¹⁸⁰), a boronic acid group (—B(OH)₂), a cyano group (—C≡N), a carboxylic acid group (—COOH), a carboxylic acid ester group (—COOR¹⁸¹), a carboxylic acid anhydride group (—CO—O—CO—R¹⁸²), a hydroxamic acid group (—CO—NH(OH)), a N-substituted hydroxamic acid group (—CO—NR¹⁸³(OH)), a O-substituted hydroxamic acid group (—CO—NH(OR¹⁸⁴)), a carboxamide group (—CO—NH₂), a N-substituted or N,N-disubstituted carboxylic acid amide group, (—CO—NHR¹⁸⁵; —CO—NR¹⁸⁶, R¹⁸⁷), an amido group (—HN—CO—R¹⁸⁸), a sulfonic acid group (—SO₃H), a sulfonamide group (—SO₂—NH₂), a N-substituted or N,N-disubstituted sulfonamide group (—SO₂—NHR¹⁸⁹; —SO₂—NR¹⁹⁰R¹⁹¹), an amidosulfone group (—NH—SO₂—R¹⁹²), a sulfone group (—SO₂—R¹⁹³), a phosphoric acid group (—OP(═O)(OH)₂), a phosphoric acid ester group (—OP(═O)(OR¹⁹⁴)(OR¹⁹⁵)), a phosphonic acid group (—P(═O)(OH)₂), an phosphonic acid ester group (—P(═O)(OR¹⁹⁶)(OR¹⁹⁷)), a halogen atom, a trifluormethyl group (—CF₃), a thiol group (—SH); a thioether group (—S—R¹¹⁹⁸), a hydroxy group (—OH); an alkoxy group (—O—R¹⁹⁹), a tetrazole group, an amino group (—NH₂), or a N-substituted or N,N-disubstituted amino group (—NHR²⁰⁰; —NR²⁰R²⁰²); and

which, independently of each other, can be substituted with one or more substituents, which can be the same or different; and,

wherein optionally, the pairs R¹⁸⁶/R¹⁸⁷, R¹⁹⁰/R¹⁹¹, R¹⁹⁴/R¹⁹⁵, R¹⁹⁶/R¹⁹⁷, and R²⁰¹/R²⁰²independenly of each other, may form a part of a ring; and

wherein the substituents R¹⁸⁰, R¹⁸¹, R¹⁸², R¹⁸³, R¹⁸⁴, R¹⁸⁵, R¹⁸⁶, R¹⁸⁷, R¹⁸⁸, R¹⁸⁹, R¹⁹⁰ R¹⁹¹, R¹⁹², R¹⁹³, R¹⁹⁴, R¹⁹⁵, R¹⁹⁶, R¹⁹⁷, R¹⁹⁸, R¹⁹⁹, R²⁰⁰, R²⁰¹, and R²⁰², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (IV)

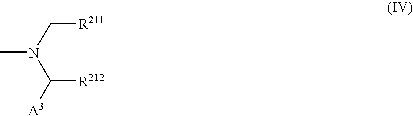

wherein R²¹¹ and R²¹², independently of each other, are

a hydrogen atom (—H); or a C₁, C₂, C₃, C₄, and C₅ branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group or, an amino group (—NH₂), or a N-substituted or N,N-disubstituted amino group (—NHR²⁴⁰; —NR²⁴¹R²⁴²); and

which, independently of each other, can be substituted with one or more substituents, which can be the same or different; and,

wherein optionally, the pair R²¹¹/R²¹², as well the pairs R²²⁶/R²²⁷, R²³⁰/R²³¹, R²³⁴/R²³⁵, R²³⁶/R²³⁷ and R²⁴¹/R²⁴², independenly of each other, may form a part of a ring; and

wherein the substituents R²²⁰, R²²¹, R²²², R²²³, R²²⁴, R²²⁵, R²²⁶, R²²⁷, R²²⁸, R²²⁹, R²³⁰ R²³¹, R²³², R²³⁴, R²³⁵, R²³⁶, R²³⁷, R²³⁸, R²³⁹, R²⁴⁰, R²⁴¹, and R²⁴², independently of each other, are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅cycloalkyl, cyano, amido, thiol trifluoromethyl, or hydroxy group; and

wherein A³ is

a hydrogen atom (—H); or a carbaldehyde (—CHO), a ketone group (—CO—R²⁶⁰), a boronic acid group (—B(OH)₂), a cyano group (—C≡N), a carboxylic acid group (—COOH), a carboxylic acid ester group (—COOR²⁶¹), a carboxylic acid anhydride group (—CO—O—CO—R²⁶²), a hydroxamic acid group (—CO—NH(OH)), a N-substituted hydroxamic acid group (—CO—NR²⁶³(OH)), a O-substituted hydroxamic acid group (—CO—NH(OR²⁶⁴)), a carboxamide group (—CO—NH₂), a N-substituted or N,N-disubstituted carboxylic acid amide group, (—CO—NHR²⁶⁵; —CO—NR²⁶⁶R²⁶⁷), an amido group (—HN—CO—R²⁶⁸), a sulfonic acid group (—SO₃H), a sulfonamide group (—SO₂—NH₂), a N-substituted or N,N-disubstituted sulfonamide group (—SO₂—NHR²⁶⁹; —SO₂—NR²⁷⁰R²⁷¹), an amidosulfone group (—NH—SO₂—R²⁷²), a sulfone group (—SO₂—R²⁷³), a phosphoric acid group (—OP(═O)(OH)₂), a phosphoric acid ester group (—OP(═O)(OR²⁷⁴)(OR²⁷⁵)), a phosphonic acid group (—P(═O)(OH)₂), an phosphonic acid ester group (—P(═O)(OR²⁷⁶)(OR²⁷⁷)), a halogen atom, a trifluormethyl group (—CF₃), a thiol group (—SH); a thioether group (—S—R²⁷⁸), a hydroxy group (—OH); an alkoxy group (—O—R²⁷⁹), a tetrazole group, an amino group (—NH₂), or a N-substituted or N,N-disubstituted amino group (—NHR²⁸⁰; —NR²⁸¹R²⁸²); and

which, independently of each other, can be substituted with one or more substituents, which can be the same or different; and,

wherein optionally, the pairs R²⁶⁶/R²⁶⁷, R²⁷⁰/R²⁷¹, R²⁷⁴/R²⁷⁵, R²⁷⁶/R²⁷⁷ and R²⁸¹/R²⁸², independenly of each other, may form a part of a ring; and

wherein the substituents R²⁶⁰, R²⁶¹, R²⁶², R²⁶³, R²⁶⁴, R²⁶⁵, R²⁶⁶, R²⁶⁷, R²⁶⁸, R²⁶⁹, R²⁷⁰ R²⁷¹, R²⁷², R²⁷³, R²⁷⁴, R²⁷⁵, R²⁷⁶, R²⁷⁷, R²⁷⁸, R²⁷⁹, R²⁸⁰, R²⁸¹, and R²⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (V)

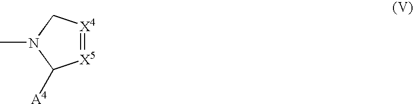

wherein X⁴ is CR²⁹¹ or N; and

wherein X⁵ is CR²⁹² or N; and

wherein R²⁹¹ and R²⁹², independently of each other, are

a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group or an amino group (—NH₂), or a N-substituted or N,N-disubstituted amino group (—NHR³²⁰; —NR³²¹R³²²); and

which, independently of each other, can be substituted with one or more substituents, which can be the same or different; and,

wherein optionally, the the pair R²⁹¹/R²⁹², if present, as well the pairs R³⁰⁶/R³⁰⁷, R³¹⁰/R³¹¹, R³¹⁴/R³¹⁵, R³¹⁶/R³¹⁷ and R³²¹/R³²², independenly of each other, may form a part of a ring; and

wherein the substituents R³⁰⁰, R³⁰¹, R³⁰², R³⁰³, R³⁰⁴, R³⁰⁵, R³⁰⁶, R³⁰⁷, R³⁰⁸, R³⁰⁹, R³¹⁰R³¹¹, R³¹², R³¹³, R³¹⁴, R³¹⁵, R³¹⁶, R³¹⁷, R³¹⁸, R³¹⁹, R³²⁰, R³²¹ and R³²² independently of each other are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅cycloalkyl, cyano, amido, thiol trifluoromethyl, or hydroxy group; and

wherein A⁴ is

a hydrogen atom (—H); or a carbaldehyde (—CHO), a ketone group (—CO—R³⁴⁰), a boronic acid group (—B(OH)₂), a cyano group (—C≡N), a carboxylic acid group (—COOH), a carboxylic acid ester group (—COOR³⁴¹), a carboxylic acid anhydride group (—CO—O—CO—R³⁴²), a hydroxamic acid group (—CO—NH(OH)), a N-substituted hydroxamic acid group (—CO—NR³⁴³ (OH)), a O-substituted hydroxamic acid group (—CO—NH(OR³⁴⁴)), a carboxamide group (—CO—NH₂), a N-substituted or N,N-disubstituted carboxylic acid amide group, (—CO—NHR³⁴⁵; —CO—NR³⁴⁶R³⁴⁷), an amido group (—HN—CO—R³⁴⁸), a sulfonic acid group (—SO₃H), a sulfonamide group (—SO₂—NH₂), a N-substituted or N,N-disubstituted sulfonamide group (—SO₂—NHR³⁴⁹; —SO₂—NR³⁵° R³⁵¹) an amidosulfone group (—NH—SO₂—R³²), a sulfone group (—SO₂—R³⁵³), a phosphoric acid group (—OP(═O)(OH)₂), a phosphoric acid ester group (—OP(═O)(OR³⁵⁴)(OR³⁵⁵)), a phosphonic acid group (—P(═O)(OH)₂), an phosphonic acid ester group (—P(═O)(OR³⁵⁶)(OR³⁵⁷)), a halogen atom, a trifluormethyl group (—CF₃), a thiol group (—SH); a thioether group (—S—R³⁵⁸), a hydroxy group (—OH); an alkoxy group (—O—R³⁵⁹), a tetrazole group, an amino group (—NH₂), or a N-substituted or N,N-disubstituted amino group (—NHR³⁶⁰; —NR³⁶¹R³⁶²); and

which, independently of each other, can be substituted with one or more substituents, which can be the same or different; and,

wherein optionally, the pairs R³⁴⁶/R³⁴⁷, R³⁵⁰/R³⁵¹, R³⁵⁴/R³⁵⁵, R³⁵⁶/R³⁵⁷ and R³⁶¹/R³⁶² independenly of each other, may form a part of a ring; and

wherein the substituents R³⁴⁰, R³⁴¹, R³⁴², R³⁴³, R³⁴⁴, R³⁴⁵, R³⁴⁶, R³⁴⁷, R³⁴⁸, R³⁴⁹, R³⁵⁰, R³⁵¹, R³⁵², R³⁵³, R³⁵⁴, R³⁵⁵, R³⁵⁶, R³⁵⁷, R³⁵⁸, R³⁵⁹, R³⁶⁰, R³⁶¹ and R³⁶²independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (VI)

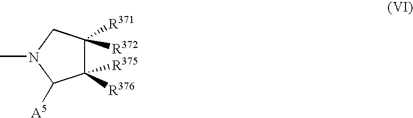

wherein R³⁷¹, R³⁷², R³⁷⁵ and R³⁷⁶, independently of each other, a hydrogen atom (—H); or a C₁, C₂, C₃, C₄, and C₅ branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, and aryl, heteroaryl, aryl-alkyl, heteroaryl-alkyl, aryl-heteroalkyl, heteroaryl-heteroalkyl group or, a carbaldehyde (—CHO), a ketone group (—CO—R³⁸⁰), a boronic acid group (—B(OH)₂), a cyano group (—C≡N), a carboxylic acid group (—COOH), a carboxylic acid ester group (—COOR³⁸¹), a halogen atom, a trifluormethyl group (—CF₃), a thiol group (—SH); a thioether group (—S—R³⁹⁸), a hydroxy group (—OH); an alkoxy group (—O—R³⁹⁹), a tetrazole group, an amino group (—NH₂), or a N-substituted or N,N-disubstituted amino group (—NHR⁴⁰⁰; —NR⁴⁰¹R⁴⁰²); and

which, independently of each other, can be substituted with one or more substituents, which can be the same or different; and,

wherein optionally, any two of the groups R³⁷¹, R³⁷², R³⁷⁵, and R³⁷⁶, as well as the pairs R¹³⁸⁶/R³⁸⁷, R³⁹⁰/R³⁹¹, R³⁹⁴/R³⁹⁵, R³⁹⁶/R³⁹⁷ and R⁴⁰¹/R⁴⁰², independenly of each other, may form a part of a ring; and

wherein the substituents R³⁸⁰, R³⁸¹, R³⁸², R³⁸³, R³⁸⁴, R³⁸⁵, R³⁸⁶, R³⁸⁷, R³⁸⁸, R³⁸⁹, R³⁹⁰, R³⁹¹, R³⁹², R³⁹³, R³⁹⁴, R³⁹⁵, R³⁹⁶, R³⁹⁷, R³⁹⁸, R³⁹⁹, R⁴⁰⁰, R⁴⁰¹, and R⁴⁰², independently of each other are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅cycloalkyl, cyano, amido, thiol trifluoromethyl, or hydroxy group; and

alternatively; the two groups R³⁷¹ and R³⁷² can be together an oxo (═O) or hydroxyimino (═N—OH) group; and

alternatively; the two groups R³⁷⁵ and R³⁷⁶ can be together an oxo (═O) or hydroxyimino (═N—OH) group; and

wherein A⁵ is

a hydrogen atom (—H); or a carbaldehyde (—CHO), a ketone group (—CO—R⁴²⁰), a boronic acid group (—B(OH)₂), a cyano group (—C≡N), a carboxylic acid group (—COOH), a carboxylic acid ester group (—COOR⁴²¹), a carboxylic acid anhydride group (—CO—O—CO—R⁴²²), a hydroxamic acid group (—CO—NH(OH)), a N-substituted hydroxamic acid group (—CO—NR⁴²³(OH)), a O-substituted hydroxamic acid group (—CO—NH(OR⁴²⁴)), a carboxamide group (—CO—NH₂), a N-substituted or N,N-disubstituted carboxylic acid amide group, (—CO—NHR⁴²⁵; —CO—NR⁴²⁶, R⁴²⁷), an amido group (—HN—CO—R⁴²⁸), a sulfonic acid group (—SO₃H), a sulfonamide group (—SO₂—NH₂), a N-substituted or N,N-disubstituted sulfonamide group (—SO₂—NHR⁴²⁹; —SO₂—NR⁴³⁰R⁴³¹), an amidosulfone group (—NH—SO₂—R⁴³²), a sulfone group (—SO₂—R⁴³³), a phosphoric acid group (—OP(═O)(OH)₂), a phosphoric acid ester group (—OP(═O)(OR⁴³⁴)(OR⁴³⁵)), a phosphonic acid group (—P(═O)(OH)₂), an phosphonic acid ester group (—P(═O)(OR⁴³⁶)(OR⁴³⁷)), a halogen atom, a trifluormethyl group (—CF₃), a thiol group (—SH); a thioether group (—S—R⁴³⁸), a hydroxy group (—OH); an alkoxy group (—O—R⁴³⁹), a tetrazole group, an amino group (—NH₂), or a N-substituted or N,N-disubstituted amino group (—NHR⁴⁴⁰; —NR⁴⁴¹R¹²); and

which, independently of each other, can be substituted with one or more substituents, which can be the same or different; and,

wherein optionally, the pairs R⁴²⁶¹R⁴²⁷, R⁴³⁰/R⁴³¹, R⁴³⁴/R⁴³⁵, R⁴³⁶/R⁴³⁷ and R⁴⁴¹/R⁴⁴², independenly of each other, may form a part of a ring; and

wherein the substituents R⁴²⁰, R⁴²¹, R⁴²², R⁴²³, R⁴²⁴, R⁴²⁵, R⁴²⁶, R⁴²⁷, R⁴²⁸, R⁴²⁹, R⁴³⁰R⁴³¹, R⁴³², R⁴³³, R⁴³⁴, R⁴³⁵, R⁴³⁶, R⁴³⁷, R⁴³⁸, R⁴³⁹, R⁴⁴⁰, R⁴⁴¹, and R⁴⁴², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (VII)

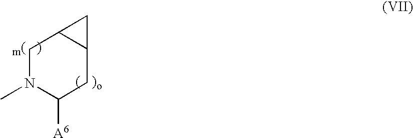

wherein m is equal to 1 or 2, and o is equal to 1 or 2, and m or o can be equal to 0;

wherein A⁶ is a hydrogen atom (—H); or a carbaldehyde (—CHO), a ketone group (—CO—R⁴⁶⁰), a boronic acid group (—B(OH)₂), a cyano group (—C≡N), a carboxylic acid group (—COOH), a carboxylic acid ester group (—COOR⁴⁶¹), a carboxylic acid anhydride group (—CO—O—CO—R⁴⁶²), a hydroxamic acid group (—CO—NH(OH)), a N-substituted hydroxamic acid group (—CO—NR⁴⁶³(OH)), a O-substituted hydroxamic acid group (—CO—NH(OR⁴⁶⁴)), a carboxamide group (—CO—NH₂), a N-substituted or N,N-disubstituted carboxylic acid aniide group, (—CO—NHR⁴⁶⁵; —CO—NR⁴R⁴⁶⁷), an amido group (—HN—CO—R⁴⁶⁸), a sulfonic acid group (—SO₃H), a sulfonamide group (—SO₂—NH₂), a N-substituted or N,N-disubstituted sulfonamide group (—SO₂—NHR⁴⁶⁹; —SO₂—NR⁴⁷⁰ an amidosulfone group (—NH—SO₂—R⁴⁷²), a sulfone group (—SO₂—R⁴⁷³), a phosphoric acid group (—OP(═O)(OH)₂), a phosphoric acid ester group (—OP(═O)(OR⁴⁷⁴)(OR⁴⁷⁵)), a phosphonic acid group (—P(═O)(OH)₂), an phosphonic acid ester group (—P(═O)(OR⁴⁷⁶)(OR⁴⁷⁷)), a halogen atom, a trifluormethyl group (—CF₃), a thiol group (—SH); a thioether group (—S—R⁴⁷⁸), a hydroxy group (—OH); an alkoxy group (—O—R⁴⁷⁹), a tetrazole group, an amino group (—NH₂), or a N-substituted or N,N-disubstituted amino group (—NHR⁴⁸⁰; —NR⁴⁸¹R⁴⁸²);

which, independently of each other, can be substituted with one or more substituents, which can be the same or different; and,

wherein optionally, the pairs R⁴⁶⁶/R⁴⁶⁷, R⁴⁷⁰/R⁷¹, R⁴⁷⁴/R⁴⁷⁵, R⁴⁷⁶/R⁴⁷⁷ and R⁴⁸/R⁴⁸² independenly of each other, may form a part of a ring; and

wherein the substituents R⁴⁶⁰, R⁴⁶¹, R⁴⁶², R⁴⁶³, R⁴⁶⁴, R⁴⁶⁵, R⁴⁶⁶, R⁴⁶⁷, R⁴⁶⁸, R⁴⁶⁹, R⁴⁷⁰, R⁴⁷¹, R⁴⁷², R⁴⁷³, R⁴⁴, R⁴⁷⁴, R⁴⁷⁵, R⁴⁷⁶, R⁴⁷⁷, R⁴⁷⁸, R⁴⁷⁹, R⁴⁸⁰, R⁴⁸¹, and R⁴⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (VIf)

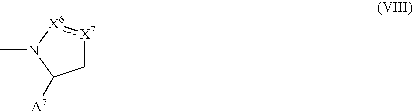

wherein x⁶ is selected from CR⁴⁹⁰R⁴⁹¹, O, S or NR⁴⁹², when the bond between x and X is a single bond; and

wherein X⁷ is selected from CR⁴⁹³R⁴⁹⁴, O, S, or NR⁴⁹⁵, when the bond between X⁶ and X⁷ is a single bond;

or alternatively,

wherein X⁶ is selected from CR⁴⁹⁶ or N, when the bond between X⁶ and X⁷ is a double bond; and

wherein X⁷ is selected from CR⁴⁹⁷ or N, when the bond between X⁶ and X⁷ is a double bond; and

wherein R⁴⁹⁰, R⁴⁹¹, R⁴⁹², R⁴⁹³, R⁴⁹⁴, R⁴⁹⁵, R⁴⁹⁶, and R⁴⁹⁷, independently of each other, are a hydrogen atom (—H); or a C₁, C₂, C₃, C₄, and C₅branched or straight chain alkyl, C₂, C₃, C₄, C₅, branched or straight chain alkenyl, C₂, C₃, C₄, Cs, branched or straight chain alkinyl, C₃, C₄, Cs, C₆, and C₇cycloalkyl, aryl, heteroaryl group, or an amino group (—NH₂), or a N-5 substituted or N,N-disubstituted amino group (—NHR⁵²⁰; —NR⁵²¹R⁵²²); and

which, independently of each other, can be substituted with one or more substituents, which can be the same or different; and,

wherein optionally, any two the groups R⁴⁹⁰, R⁴⁹¹, R⁴⁹², R⁴⁹³, R⁴⁹⁵, R⁴⁹⁶, and R⁴⁹⁷, if present, as well as the pairs R⁵⁰⁶/R⁵⁰⁷, R⁵¹⁰/R⁵¹¹, R⁵¹⁴/R⁵¹⁵, R⁵¹⁶/R⁵¹⁷ and R⁵²¹/R⁵²², independenly of each other, may form a part of a ring; and

wherein the substituents R⁵⁰⁰, R⁵⁰¹, R⁵⁰², R⁵⁰³, R⁵⁰⁴, R⁵⁰⁵, R⁵⁰⁶, R⁵⁰⁷, R⁵⁰⁸, R⁵⁰⁹, R⁵¹⁰ R⁵¹¹, R⁵¹², R⁵¹³, R⁵¹⁴, R⁵¹⁶, R⁵¹⁷, R⁵¹⁸, R⁵¹⁹, R⁵²⁰, R⁵²¹, and R⁵²², independently of each other are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅ branched or straight chain alkenoxy, phenyloxy, benzyloxy, C₃, C₄, C₅ cycloalkyl, cyano, amido, thiol trifluoromethyl, or hydroxy group; and

wherein A⁷ is

a hydrogen atom (—H); or a carbaldehyde (—CHO), a ketone group (—CO—R⁵⁴⁰), a boronic acid group (—B(OH)₂), a cyano group (—C≡N), a carboxylic acid group (—COOH), a carboxylic acid ester group (—COOR⁵⁴¹), a carboxylic acid anhydride group (—CO—O—CO—R⁵⁴²), a hydroxamic acid group (—CO—NH(OH)), a N-substituted hydroxamic acid group (—CO—NR⁵⁴³ (OH)), a O-substituted hydroxamic acid group (—CO—NH(OR⁵⁴⁴)), a carboxamide group (—CO—NH₂), a N-substituted or N,N-disubstituted carboxylic acid amide group, (—CO—NHR⁵⁴⁵; —CO—NR⁵⁴⁶R⁵⁴⁷), an amido group (—HN—CO—R⁵⁴⁸), a sulfonic acid group (—SO₃H), a sulfonamide group (—SO₂—NH₂), a N-substituted or N,N-disubstituted sulfonamide group (—SO₂—NHR⁵⁴⁹; —SO₂—NR⁵⁵⁰R⁵⁵¹), an amidosulfone group (—NH—SO₂—R⁵⁵²), a sulfone group (—SO₂—R⁵⁵³), a phosphoric acid group (—OP(═O)(OH)₂), a phosphoric acid ester group (—OP(═O)(OR⁵⁵⁴)(OR⁵⁵⁵)), a phosphonic acid group (—P(═O)(OH)₂), an phosphonic acid ester group (—P(═O)(OR⁵⁵⁶)(OR⁵⁵⁷)), a halogen atom, a trifluormethyl group (—CF₃), a thiol group (—SH); a thioether group (—S—R⁵⁵⁸), a hydroxy group (—OH); an alkoxy group (—O—R⁵⁵⁹), a tetrazole group, an amino group (—NH₂), or a N-substituted or N,N-disubstituted amino group (—NHR⁵⁶⁰; —NR⁵⁶¹R⁵⁶²); and

which, independently of each other, can be substituted with one or more substituents, which can be the same or different; and,

wherein optionally, the pairs R⁵⁴⁶/R⁵⁴⁷, R⁵⁵⁰/R⁵⁵¹, R⁵⁵⁴/R⁵⁵⁵, R⁵⁵⁶/R⁵⁵⁷ and R⁵⁶¹/R⁵⁶², independenly of each other, may form a part of a ring; and

wherein the substituents R⁵⁴⁰, R⁵⁴¹, R⁵⁴², R⁵⁴³, R⁵⁴⁴, R⁵⁴⁵, R⁵⁴⁶, R⁵⁴⁷, R⁵⁴⁸, R⁵⁴⁹, R⁵⁵⁰, R⁵⁵¹, R⁵⁵², R⁵⁵³, R⁵⁵⁴, R⁵⁵⁵, R⁵⁵⁶, R⁵⁵⁷, R⁵⁵⁸, R⁵⁵⁹, R⁵⁶⁰, R⁵⁶¹, and R⁵⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (IX) or (IXa)

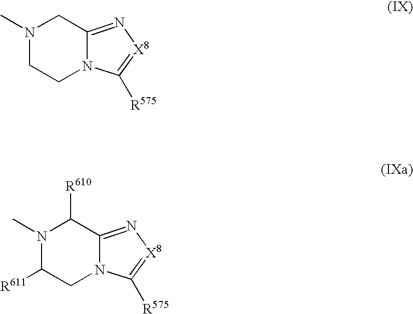

wherein X⁸ is N or CR⁵⁷⁰; and

wherein R⁵⁷⁰, R⁵⁷⁵, R⁶¹⁰ and R⁶¹¹ independently of each other, are a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group, or a carbaldehyde (—CHO), a ketone group (—CO—R⁵⁸⁰), a boronic acid group (—B(OH)₂), a cyano group (—C≡N), a carboxylic acid group (—COOH), a carboxylic acid ester group (—COOR⁵⁸¹), a carboxylic acid anhydride group (—CO—O—CO—R⁵⁸²), a hydroxamic acid group (—CO—NH(OH)), a N-substituted hydroxamic acid group (—CO—NR⁵⁸³ (OH)), a O-substituted hydroxamic acid group (—CO—NH(OR⁵⁸⁴)), a carboxamide group (—CO—NH₂), a N-substituted or N,N-disubstituted carboxylic acid amide group, (—CO—NHR⁵⁸⁵; —CO—NR⁵⁸⁶R⁵⁸⁷), an amido group (—HN—CO—R⁵⁸⁸), a sulfonic acid group (—SO₃H), a sulfonamide group (—SO₂—NH₂), a N-substituted or N,N-disubstituted sulfonamide group (—SO₂—NHR⁵⁸⁹; —SO₂—NR⁵⁹OR⁵⁹¹), an amidosulfone group (—NH—SO₂—R⁵⁹²), a sulfone group (—SO₂—R⁵⁹³), a phosphoric acid group (—OP(═O)(OH)₂), a phosphoric acid ester group (—OP(═O)(OR⁵⁹⁴)(OR⁵⁹⁵)), a phosphonic acid group (—P(═O)(OH)₂), an phosphonic acid ester group (—P(═O)(OR⁵⁹⁶)(OR⁵⁹⁷)), a halogen atom, a trifluormethyl group (—CF₃), a thiol group (—SH); a thioether group (—S—R⁵⁹⁸), a hydroxy group (—OH); an alkoxy group (—O—R⁵⁹⁹), a tetrazole group, an amino group (—NH₂), or a N-substituted or N,N-disubstituted amino group (—NHR⁶⁰⁰; —NR⁶⁰¹R⁶⁰²);

which, independently of each other, can be substituted with one or more substituents, which can be the same or different; and,

wherein optionally, the pairs R⁵⁷⁰/R⁵⁷⁵, if present, as well as the pairs R⁵⁸⁶/R⁵⁸⁷, R⁵⁹⁰/R⁵⁹¹, R⁵⁹⁴/R⁵⁹⁵, R⁵⁹⁶/R⁵⁹¹ and R⁶⁰¹/R⁶⁰², independenly of each other, may form a part of a ring; and

wherein the substituents R⁵⁸⁰, R⁵⁸¹, R⁵⁸², R⁵⁸³, R⁵⁸⁴, R⁵⁸⁵, R⁵⁸⁶, R⁵⁸⁷, R⁵⁸⁸, R⁵⁸⁹, R⁵⁹⁰, R⁵⁹¹, R⁵⁹², R⁵⁹³, R⁵⁹⁴, R⁵⁹⁵, R⁵⁹⁶, R⁵⁹⁷, R⁵⁹⁸, R⁵⁹⁹, R⁶⁰⁰, R⁶⁰¹, and R⁶⁰², independently of each other are a hydrogen atom (—H), or a C₁, C₂, C₃, C₄, and C₅ branched or straight chain alkyl, aryl, heteroaryl, amino, halo, carbonyl, C₁, C₂, C₃, C₄, C₅, branched or straight chain alkoxy, C₂, C₃, C₄, C₅ branched or straight chain alkenoxy, phenyloxy, benzyloxy, C₃, C₄, C₅ cycloalkyl, cyano, amido, thiol trifluoromethyl, or hydroxy group; and

or wherein the group PM

has the formula (X)

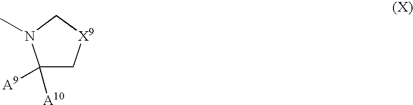

wherein the groups X⁹ is CR⁹⁰⁰R⁹⁰¹, S, SO, SO₂ or NR⁹⁰²

wherein R⁹⁰⁰, R⁹⁰¹ and R⁹⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR⁹¹⁰R⁹¹¹.

wherein A⁹ and A¹⁰ are, independently of each other, selected from hydrogen, cyano, —C(═O)NR⁹¹²R⁹¹³, or C₁, C₂, C₃, C₄, C₅or C₆alkyl, which is linear or branched and is optionally substituted with 1, 2, 3, 4, or 5 halogens;

wherein

R⁹¹⁰ and R⁹¹², are, independently of each other, selected from hydrogen, or C₁, C₂, C₃, C₄, C₅or C₆alkyl, which is linear or branched and is optionally substituted with 1, 2, 3, 4, or 5 halogens; and

R⁹¹¹ and R⁹¹³, are, independently of each other, selected from the group consisting of

(1) phenyl, which is optionally substituted with 1, 2, 3, 4, or 5, substituents independently selected from halogen and R⁹²⁰;

(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of

(a) hydroxy,

(b) —COOH,

(c) —COO(C₁, C₂, C₃, C₄, C₅or C₆alkyl), i.e. ester,

(d) phenyl,

(e) naphthyl,

(f) C₃, C₄, C₅or C₆cycloalkyl,

(g) a 5- or 6 membered heterocycle which may be saturated or unsaturated comprising 1, 2, 3, or 4 heteroatoms independently selected from nitrogen, oxygen or sulfur;

(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;

wherein said C₃, C₄, Cs or C₆cycloalkyl, phenyl, naphthyl, are optionally substituted with 1, 2, 3, 4, or 5 substituents independently selected from halogen 920 and R⁹²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R⁹²⁰; and

(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;

wherein R⁹²⁰ is selected from the group consisting of:

(1) hydroxy;

(2) cyano;

(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;

(4) C₁, C₂, C₃, C₄, C₅, C₆, C₇, C₈, C₉ or C₁₀ alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from

(a) hydroxy;

(b) —COOH;

(c) —COO(C₁, C₂, C₃, C₄, C₅or C₆alkyl) i.e. ester, which may linear or branched and is optionally substituted with 1, 2, 3, 4, or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR⁹²⁵R⁹²⁵;

(g) —SO₂NR⁹²⁵R⁹²⁵

(h) —NR⁹²⁵—C(═O)R⁹²⁵

(i) —NR⁹²⁵—C(═O)NR⁹²⁵R⁹²⁵;

(k) —O—CO—R⁹³⁰

(l) —O—CO—NR⁹²⁵R⁹²⁵;

(m) —NR⁹²⁵SO₂R⁹³⁰;

(n) NR⁹²⁵R⁹²⁵;

(o) phenyl which is optionally substituted with 1, 2, 3, 4, or 5 group independently selected from halogen, hydroxy, C₁, C₂, C₃, C₄, Cs or C₆alkyl, —OC₁, —OC₂, —OC₃, —OC₄, —OC₅or —OC₆alkyl, —COOH, —COO(C₁, C₂, C₃, C₄, Cs or C₆alkyl) i.e. ester, said C₁, C₂, C₃, C₄, Cs or C₆alkyl, —OC₁, —OC₂, —OC₃, —OC₄, —OC₅or —OC₆alkyl, —COOH, —COO(C₁, C₂, C₃, C₄, C₅ or C₆alkyl) i.e. ester being linear or branched and optionally substituted with 1, 2, 3, 4, 5, or 6 substitutents independently selected from 0 or 1 C₃, C₄Cs or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(5) OC₁, OC₂, OC₃, OC₄, OC₅, OC₆, OC₇, OC₈, OC₉or OC₁₀alkyl, which is linear or branched and is optionally substituted with 0, 1, 2, 3, 4, or 5 halogen atoms and 0, 1, or 2 substitutents selected from

(a) hydroxy;

(b) —COOH;

(c) —COO(C₁, C₂, C₃, C₄, C₅or C₆alkyl) i.e. ester, which may be linear or branched and is optionally substituted with 1, 2, 3, 4 or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅ or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR⁹²⁵R⁹²⁵;

(g) —SO₂NR⁹²⁵R⁹²⁵;

(h) —NR²⁵—C(═O)R⁹²¹

(i) —NR⁹²⁵—C(═O)NR⁹²⁵, R⁹²⁵

(j) —NR⁹²⁵ COOR⁹³⁰

(k) —O—CO—R⁹³⁰

(l) —O—CO—NR⁹²⁵ R⁹²⁵;

(m) —NR⁹²⁵SO₂R⁹³⁰;

(n) NR⁹²⁵R⁹²⁵;

(o) phenyl, which is optionally substituted with 1, 2, 3, 4, or 5 groups independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(6) —COOH;

(7) —COO(C₁, C₂, C₃, C₄, C₅or C₆alkyl) i.e. ester, which may be linear or branched and is optionally substituted with 1, 2, 3, 4, 5 halogens;

(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.

(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(10)—CONR⁹²⁵R⁹²⁵;

(11) —SO₂NR⁹²⁵R⁹²⁵;

(12) —NR⁹²—C(═O)R⁹²⁵

(13) —NR²⁵—C(═O)NR⁹²⁵R⁹²⁵;

(14) —NR⁹²⁵COOR⁹³⁰

(15)—O—CO—R⁹³⁰ (16)—O—CO—NR⁹²⁵, R⁹²⁵;

(17) —NR⁹²⁵SO₂R⁹³⁰;

(18) —NR⁹²⁵R⁹²⁵;

(19) phenyl, which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;

wherein R⁹³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.

wherein R⁹²⁵ is selected from R⁹³⁰ and hydrogen.

wherein the group PM

has the formula (XI)

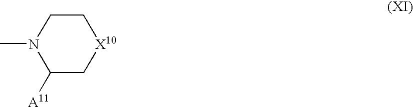

wherein the groups X¹⁰ is CR¹⁰⁰⁰R¹⁰⁰¹, S, SO, SO₂or NR¹⁰⁰²

wherein R¹⁰⁰⁰, R¹⁰⁰¹ and R¹⁰⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR¹⁰¹⁰R¹⁰¹¹.

and A¹¹ is selected from

hydrogen, cyano, —C(═O)NR¹⁰¹²R¹⁰¹³, or C₁, C₂, C₃, C₄, C₅or C₆alkyl, which is linear or branched and is optionally substituted with 1, 2, 3, 4, or 5 halogens;

wherein

R¹⁰¹⁰ and R¹⁰¹², are, independently of each other, selected from hydrogen, or C₁, C₂, C₃, C₄, C₅or C₆alkyl, which is linear or branched and is optionally substituted with 1, 2, 3, 4, or 5 halogens; and

R¹⁰¹¹ and R¹⁰¹³, are, independently of each other, selected from the group consisting of

(1) phenyl, which is optionally substituted with 1, 2, 3, 4, or 5, substituents independently selected from halogen and R¹⁰²⁰;

(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of

(a) hydroxy,

(b) —COOH,

(c) —COO(C₁, C₂, C₃, C₄, C₅or C₆alkyl), i.e. ester,

(d) phenyl,

(e) naphthyl,

(f) C₃, C₄, C₅or C₆cycloalkyl,

(g) a 5- or 6 membered htereocycle which may be saturated or unsaturated comprising 1, 2, 3, or 4 heteroatoms independently selected from nitrogen, oxygen or sulfur;

(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;

wherein said C₃, C₄, C₅or C₆cycloalkyl, phenyl, naphthyl, are optionally substituted with 1, 2, 3, 4, or 5 substituents independently selected from halogen and R¹⁰²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R¹⁰²⁰; and

(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;

wherein R¹⁰²⁰ is selected from the group consisting of:

(1) hydroxy;

(2) cyano;

(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;

(4) C₁, C₂, C₃, C₄, C₅, C₆, C₇, C₈, Cg or C₁₀alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from

(a) hydroxy;

(b) —COOH;

(c) —COO(C₁, C₂, C₃, C₄, C₅or C₆alkyl) i.e. ester, which may linear or branched and is optionally substituted with 1, 2, 3, 4, or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, Cs or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR¹⁰²⁵, R¹⁰²⁵;

(g) —SO₂NR¹⁰²⁵R¹⁰²⁵;

(h) —NR¹¹²⁵—C(═O)R¹¹²¹

(i) —NR¹⁰²⁵—C(═O)NR¹⁰²⁵R¹⁰²⁵R

(j) —NR¹⁰²⁵ COOR¹⁰³⁰

(k) —O—CO—R¹⁰³⁰

(l) —O—CO—NR¹⁰²⁵R¹⁰²⁵;

(m) —NR¹⁰²⁵SO₂R¹⁰³⁰;

(n) —NR¹⁰²⁵R¹⁰²⁵;

(o) phenyl which is optionally substituted with 1, 2, 3, 4, or 5 group independently selected from halogen, hydroxy, C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(5) OC₁, OC₂, OC₃, OC₄, OC₅, OC₆, OC₇, OC₈, OC₉or OC₁₀alkyl, which is linear or branched and is optionally substituted with 0, 1, 2, 3, 4, or 5 halogen atoms and 0, 1, or 2 substitutents selected from

(a) hydroxy;

(b) —COOH;

(c) —COO(C₁, C₂, C₃, C₄, C₅or C₆alkyl) i.e. ester, which may be linear or branched and is optionally substituted with 1, 2, 3, 4 or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR¹⁰²⁵R¹⁰²⁵;

(g) —SO₂NR¹⁰²⁵R¹⁰²⁵;

(h) —NR¹⁰²⁵—C(═O)R¹¹⁰²⁵

(i) —NR¹⁰²⁵C(═O)NR¹⁰²⁵R¹⁰²⁵;

(j) —NR¹¹¹²⁵COOR¹⁰³⁰ (k) —O—CO—R¹⁰³⁰

(l) —O—CO—NRO25, R¹⁰²⁵;

(m) —NR¹⁰²⁵SO₂R¹⁰³⁰;

(n) —NR¹⁰²⁵R¹⁰²⁵;

(o) phenyl, which is optionally substituted with 1, 2, 3, 4, or 5 groups independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(6) —COOH;

(7) —COO(C₁, C₂, C₃, C₄, C₅or C₆alkyl) i.e. ester, which may be linear or branched and is optionally substituted with 1, 2, 3, 4, 5 halogens;

(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.

(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(10) —CONR¹⁰²⁵, R¹⁰²⁵

(11)—SO₂NR¹⁰²⁵R¹⁰²⁵

(12)—NR¹⁰²⁵—C(═O)R¹¹²⁵

(13)—NR¹⁰²⁵—C(═O)NR¹⁰²⁵, R¹⁰²⁵

(14)—NR⁹²⁵COOR¹⁰³⁰

(15)—O—CO—R¹⁰³⁰

(16)—O—CO_NR¹⁰²⁵R¹⁰²⁵;

(17) —NR¹⁰²⁵ SO₂R¹⁰³⁰;

(18)—NR¹⁰²⁵R¹⁰²⁵;

(19) phenyl, which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;

wherein R¹⁰³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or Cs alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.

wherein R¹⁰²⁵ is selected from R¹⁰³⁰ and hydrogen.

or wherein the group PM

has the formula (XII)

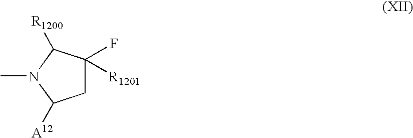

wherein the groups R¹²⁰¹ is hydrogen orfluoro.

wherein R¹²⁰⁰ und A¹² is selected from hydrogen and cyano, and the other is hydrogen.

or wherein the group PM

has the formula XM:

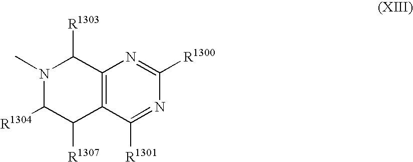

wherein:

—R¹³⁰⁰ is selected from the group consisting of:

(1) hydrogen,

(2) CN,

(3) C₁₋₁₀alkyl, which is linear or branched and which is unsubstituted or substituted with:

a) halogen, or

b) phenyl, which is unsubstituted or substituted with 1-5 substitutents independently selected from halogen, CN, OH, R¹³⁰², OR¹³⁰², NHSO₂R¹³⁰², N(C₁₋₆ alkyl)SO₂R¹³⁰², SO₂R¹³⁰², SO₂NR¹³⁰²R¹³⁰², NR¹³⁰⁵R¹³⁰⁶, CONR¹³⁰⁵R¹³⁰⁶ CO₂H, and CO₂C₁₋₆alkyl, wherein the C₁₋₆alkyl is linear or branched,

(4) phenyl which is unsubstituted or substituted with 1-5 substitutents independently selected from halogen, CN, OH, R¹³⁰², OR¹³⁰², NHSO₂R¹³⁰², N(C₁₋₆alkyl)SO₂R¹³⁰², SO₂R¹³⁰², SO₂NR¹³⁰⁵R¹³⁰⁶, NR¹³⁰⁵R¹³⁰⁶, CONR¹³⁰⁵R¹³⁰⁶, CO₂H, and CO₂C₁₋₆alkyl, wherein the C₁₋₆alkyl is linear or branched,

(5) a 5- or 6-membered heterocyclic which may be saturated or unsaturated comprising 1-4 heteroatoms independently selected from N, S and O, the heterocycle being unsubstituted or substituted with 1-3 substituents independently selected from oxo, halogen, NO₂, CN, OH, R¹³⁰², OR¹³⁰², NHSO₂R¹³⁰², N(C₁₋₆alkyl)SO₂R¹³⁰², SO₂R¹³⁰² SO₂NR¹³⁰⁵R¹³⁰⁶ NR¹³⁰⁵R¹³⁰⁶, CONR¹³⁰⁵R¹³⁰⁶, CO₂H, and CO₂C₁₋₆alkyl, wherein the C₁₋₆alkyl is linear or branched,

(6) C₃₋₆cycloalkyl, which is optionally substituted with 1-5 substituents independently selected from halogen, OH, C₁₋₆alkyl, and OC₁₋₆alkyl, wherein the C₁₋₆alkyl and OC₁₋₆ alkyl are linear or branched and optionally substituted with 1-5 halogens

(7) OH

(8) OR¹³⁰², and

(9) NR¹³⁰⁵R¹³⁰⁶;

and R¹³⁰¹ is hydrogen;

R¹³⁰² is C₁₋₆alkyl, which is linear or branched and which is unsubstituted or substituted with 1-5 groups independently selected from halogen, CO₂H, and CO₂C₁₋₆alkyl, wherein the C₁₋₆alkyl is linear or branched;

R¹³⁰³ is selected from the group consisting of:

(1) hydrogen,

(2) C₁₋₁₀alkyl, which is linear or branched and which is unsubstituted or substituted with one or more substituted selected from:

a) halogen,

b) hydroxy,

c) phenyl, which is unsubstituted or substituted with 1-5 substitutents independently selected from halogen, OH, C₁₋₆alkyl, and OC₁₋₆alkyl, wherein the C₁₋₆alkyl is linear or branched and optionally substituted with 1-5 halogens,

d) naphthyl, wherein the naphthyl is optionally substituted with 1-5 substituents independently selected from halogen, OH, C₁₋₆alkyl, and OC₁₋₆alkyl, wherein the C₁₋₆alkyl is linear or branched and optionally substituted with 1-5 halogens,

e) CO₂H,

f) CO₂C₁₋₆alkyl,

g) CoNR¹³⁰⁵R¹³⁰⁶

(3) CN,

(4) phenyl which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens,

(5) naphthyl which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens,

(6) CO₂H,

(7) CO₂C₁₋₆alkyl,

(8) CONR¹³⁰⁵R¹³⁰⁶, and

(9) C₃₋₆cycloalkyl, which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens

R¹³⁰⁵ and R¹³⁰⁶ are independently selected from the group consisting of:

(1) hydrogen,

(2) phenyl, which is unsubstituted or substituted with substituents independently selected from halogen, OH, C₁₋₆alkyl, and OC₁₋₆alkyl, wherein the C₁₋₆alkyl is linear or branched and optionally substituted with 1-5 halogens (3) C₃₋₆cycloalkyl, which is unsubstituted or substituted with 1-5 substituents independently selected from C₁₋₆alkyl, and OC₁₋₆alkyl, wherein the C₁₋₆alkyl is linear or branched and optionally substituted with 1-5 halogens

(4) C₁₋₆alkyl, which is linear or branched and which is unsubstituted or substituted with:

a) halogen, or

b) phenyl, which is unsubstituted or substituted with 1-5 substituents independently selected from halogen, OH, C₁₋₆alkyl, and OC₁₋₆alkyl, wherein the C₁₋₆alkyl is linear or branched and optionally substituted with 1-5 halogens,

or wherein R¹³⁰⁵ and R¹³⁰⁶ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆alkyl, and C₁₋₆alkoxy, wherein alkyl and alkoxy are unsubstituted with one to five halogens;

R¹³⁰⁴ and R¹³⁰⁷ are hydrogen;

or wherein the group PM

has the formula XIV:

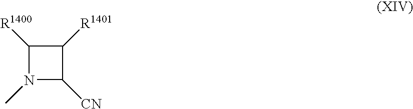

wherein R¹⁴⁰⁰ is H and R¹⁴⁰¹ is hydrogen atom (—H); or halogen, or cyano or ethynyl;

or wherein the group PM

has the formula (XV)

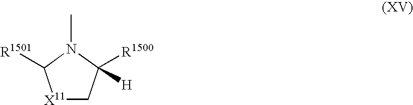

wherein X¹¹ is CH₂, CHF or CF²;

wherein R¹⁵⁰⁰ is cyano;

wherein R¹⁵⁰¹ is selected from the group consisting of alkyl, alkenyl and alkynyl;

Preferred are compounds as disclosed above

wherein the group PM

has the formula (II)

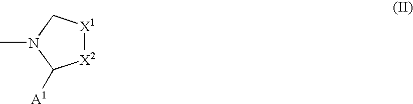

wherein X¹ is CR⁵¹R⁵² or S; and

wherein X² is CR⁵⁴R⁵⁵; and

wherein R⁵¹, R⁵², R⁵³, and R⁵⁵, independently of each other, are a hydrogen atom (—H);

wherein A¹ is

a hydrogen atom (—H), or a boronic acid group (—B(OH)₂), a cyano group (—C≡N), or a phosphonic acid ester group (—P(═O)(OR¹¹⁶)(OR¹¹⁷)),

which, independently of each other, can be substituted with one or more substituents, which can be the same or different; and,

wherein optionally, the pairs R¹¹⁶/R¹¹⁷ may form a part of a ring;

wherein the substituents R¹¹⁶ and R¹¹⁷ independently of each other, are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (III)

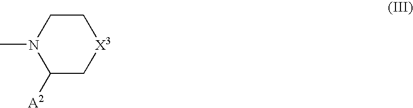

wherein X³ is CR¹³, R¹³² or S; and

wherein R¹³¹, R¹³², independently of each other, are a hydrogen atom (—H);

wherein A² is

a hydrogen atom (—H); a boronic acid group (—B(OH)₂), a cyano group (—C≡N), a phosphonic acid ester group (—P(═O)(OR¹⁹⁶)(OR¹⁹⁷));

which, independently of each other, can be substituted with one or more substituents, which can be the same or different; and,

wherein optionally, the pairs R¹⁹⁶/R¹⁹⁷ may form a part of a ring; and

wherein the substituents R¹⁹⁶ and R¹⁹⁷, independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (IV)

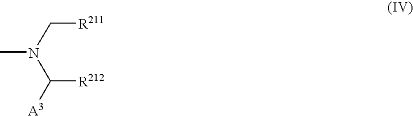

wherein R²¹¹ and R²¹², independently of each other, are

a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group or, an amino group (—NH₂), or a N-substituted or N,N-disubstituted amino group (—NHR²⁴⁰; —NR²⁴¹R²⁴²); and

which, independently of each other, can be substituted with one or more substituents, which can be the same or different; and,

wherein optionally, the pair R²¹¹/R²¹², as well the pairs R²²⁶/R²²⁷, R²³⁰/R²³¹, R²³⁴/R²³⁵, R²³⁶/R²³⁷ and R²⁴¹/R²⁴², independenly of each other, may form a part of a ring; and

wherein the substituents R²²⁰, R²²¹, R²²², R²²³, R²²⁴, R²²⁵, R²²⁶, R²²⁷, R²²⁸, R²²⁹, R²³⁰, R²³¹, R²³², R²³³, R²³⁴, R²³⁵, R²³⁶, R²³⁷, R²³⁸, R²³⁹, R²⁴⁰, R²⁴¹, and R²⁴², independently of each other, are a hydrogen atom (—H), or a C₁, C₂, C₃, C₄, and C₅ branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅cycloalkyl, cyano, amido, thiol trifluoromethyl, or hydroxy group; and

wherein A³ is

a hydrogen atom (—H); or a boronic acid group (—B(OH)₂), a cyano group (—C≡N), or a phosphonic acid ester group (—P(═O)(OR²⁷⁶)(OR²⁷⁷))

which, independently of each other, can be substituted with one or more substituents, which can be the same or different; and,

wherein optionally, the pair R²⁷⁶/R²⁷⁷ may form a part of a ring; and

wherein the substituents R²⁷⁶ and R²⁷⁷, independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (V)

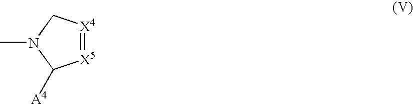

wherein X⁴ is CR²⁹¹ or N; and

wherein X⁵ is CR²⁹² or N; and

wherein R²⁹¹ and R²⁹², independently of each other, are

a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group or an amino group (—NH₂), or a N-substituted or N,N-disubstituted amino group (—NHR³²⁰; —NR³²¹R³²²); and

which, independently of each other, can be substituted with one or more substituents, which can be the same or different; and,

wherein optionally, the the pair R²⁹¹/R²⁹², if present, as well the pairs R³⁰⁶/R³⁰⁷, R³¹⁰/R³¹¹, R³¹⁴/R³¹⁵, R³¹⁶/R³¹⁷ and R³²¹/R³²², independenly of each other, may form a part of a ring; and

wherein the substituents R³⁰⁰, R³⁰¹, R³⁰², R³⁰³, R³⁰⁴, R³⁰⁵, R³⁰⁶, R³⁰⁷, R³⁰⁸, R³⁰⁹, R³¹⁰, R³¹¹, R³¹², R³¹³, R³¹⁴, R³¹⁵, R³¹⁶, R³¹⁷, R³¹⁸, R³¹⁹, R³²⁰, R²³¹, and R³²², independently of each other are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅cycloalkyl, cyano, amido, thiol trifluoromethyl, or hydroxy group; and

wherein A⁴ is

a hydrogen atom (—H); or a boronic acid group (—B(OH)₂), a cyano group (—C≡N), a phosphonic acid ester group (—P(═O)(OR³⁵⁶)(OR³⁵⁷)),

which, independently of each other, can be substituted with one or more substituents, which can be the same or different; and,

wherein optionally, the pairs R³⁵⁶/R³⁵⁷ may form a part of a ring; and

wherein the substituents R³⁵⁶ and R²⁵⁷, independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (VI)

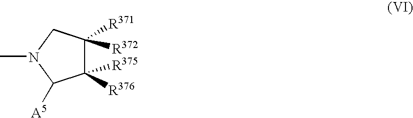

wherein R³⁷¹, R³⁷², R³⁷⁵ and R³⁷⁶, independently of each other, a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, and aryl, heteroaryl, aryl-alkyl, heteroaryl-alkyl, aryl-heteroalkyl, heteroaryl-heteroalkyl group or, a carbaldehyde (—CHO), a ketone group (—CO—R³⁸⁰), a boronic acid group (—B(OH)₂), a cyano group (—C≡N), a carboxylic acid group (—COOH), a carboxylic acid ester group (—COOR³⁸¹), a halogen atom, a trifluormethyl group (—CF₃), a thiol group (—SH); a thioether group (—S—R³⁹⁸), a hydroxy group (—OH); an alkoxy group (—O—R³⁹⁹), a tetrazole group, an amino group (—NH₂), or a N-substituted or N,N-disubstituted amino group (—NHR⁴⁰⁰; —NR⁴⁰¹R⁴⁰²); and

which, independently of each other, can be substituted with one or more substituents, which can be the same or different; and,

wherein optionally, any two of the groups R³⁷¹, R³⁷², R³⁷⁵, and R³⁷⁶, as well as the pairs R³⁸⁶/R³⁸⁷, R³⁹⁰/R³⁹¹, R³⁹⁴/R³⁹⁵, R³⁹⁶/R³⁹⁷ and R⁴⁰¹/R⁴⁰², independenly of each other, may form a part of a ring; and

wherein the substituents R³⁸⁰R³⁸¹, R³⁸², R³⁸³, R³⁸⁴, R³⁸⁵, R³⁸⁶, R³⁸⁷, R³⁸⁸, R³⁸⁹, R³⁹⁰, R³⁹¹, R³⁹², R³⁹³, R³⁹⁴, R³⁹⁵, R³⁹⁶, R³⁹⁷, R³⁹⁸, R³⁹⁹, R⁴⁰⁰, R⁴⁰¹, and R⁴⁰², independently of each other are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅ cycloalkyl, cyano, amido, thiol trifluoromethyl, or hydroxy group; and

alternatively; the two groups R³⁷¹ and R³⁷² can be together an oxo (═O) or hydroxyimino (═N—OH) group; and

alternatively; the two groups R³⁷⁵ and R³⁷⁶ can be together an oxo (═O) or hydroxyiniino (═N—OH) group; and

wherein A⁵ is

a hydrogen atom (—H); or a boronic acid group (—B(OH)₂), a cyano group (—C≡N), or a phosphonic acid ester group (—P(═O)(OR⁴³⁶)(OR⁴³⁷));

which, independently of each other, can be substituted with one or more substituents, which can be the same or different; and,

wherein optionally, the pairs R⁴³⁶/R⁴³⁷ may form a part of a ring; and

wherein the substituents R⁴³⁶ and R⁴³⁷, independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (VII)

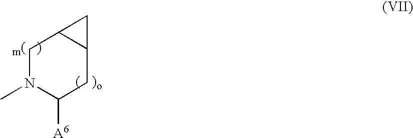

wherein m is equal to 0 and o is equal to 1, or m is equal to 1 and o is equal to 0, or m is equal to 1 and o is equal to 1, or m is equal to 2 and o is equal to 0;

wherein A⁶ is a hydrogen atom (—H); or a boronic acid group (—B(OH)₂), a cyano group (—C≡N), or a phosphonic acid ester group (—P(═O)(OR⁴⁷⁶)(OR⁴⁷⁷)),

which, independently of each other, can be substituted with one or more substituents, which can be the same or different; and,

wherein optionally, the pairs R⁴⁷⁶/R⁴⁷⁷ may form a part of a ring; and

wherein the substituents R⁴⁷⁶ and R⁴⁷⁷, independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (VIII)

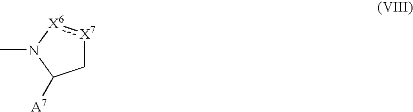

wherein X⁶ is selected from CR⁴⁹⁰R⁴⁹¹, O, S or NR⁴⁹², when the bond between X⁶ and X⁷ is a single bond; and

wherein X⁷ is selected from CR⁴⁹³R⁴⁹⁴, O, S, or NR⁴⁹⁵, when the bond between X⁶ and X⁷ is a single bond;

or alternatively,

wherein X⁶ is selected from CR⁴⁹⁶ or N, when the bond between X⁶ and X⁷ is a double bond; and

wherein X⁷ is selected from CR⁴⁹⁷ or N, when the bond between X⁶ and X⁷ is a double bond; and

wherein R⁴⁹⁰, R⁴⁹¹, R⁴⁹², R⁴⁹³, R⁴⁹⁴, R⁴⁹⁵, R⁴⁹⁶, and R⁴⁹⁷, independently of each other, are a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group, or an amino group (—NH₂), or a N-substituted or N,N-disubstituted amino group (—NHR⁵²⁰; —NR⁵²¹R⁵²²); and

which, independently of each other, can be substituted with one or more substituents, which can be the same or different; and,

wherein optionally, any two the groups R⁴⁹⁰, R⁴⁹¹, R⁴⁹², R⁴⁹³, R⁴⁹⁴, R⁴⁹⁵, R⁴⁹⁶ and R⁴⁹⁷, if present, as well as the pairs R⁵⁰⁶/R⁵⁰⁷, R⁵¹⁰/R⁵¹¹, R⁵¹⁴/R⁵¹⁵, R⁵¹⁶/R⁵¹⁷ and R⁵²¹/R⁵²², independenly of each other, may form a part of a ring; and

wherein the substituents R⁵⁰⁰, R⁵⁰¹, R⁵⁰², R⁵⁰³, R⁵⁰⁴, R⁵⁰⁵, R⁵⁰⁶, R⁵⁰⁷, R⁵⁰⁸, R⁵⁰⁹, R⁵¹⁰, R⁵¹¹, R⁵¹², R⁵¹³, R⁵¹⁴, R⁵¹⁵, R⁵¹⁶, R⁵¹⁷, R⁵¹⁸, R⁵¹⁹, R⁵²⁰, R⁵²¹, and R⁵²² independently of each other are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅cycloalkyl, cyano, amido, thiol trifluoromethyl, or hydroxy group; and

wherein A⁷ is

a hydrogen atom (—H); or a carbaldehyde (—CHO), a ketone group (—CO—R⁵⁴⁰), a boronic acid group (—B(OH)₂), a cyano group (—C≡N), a carboxylic acid group (—COOH), a carboxylic acid ester group (—COOR⁵⁴¹), a carboxylic acid anhydride group (—CO—O—CO—R⁵⁴²), a hydroxamic acid group (—CO—NH(OH)), a N-substituted hydroxamic acid group (—CO—NR⁵⁴³(OH)), a O-substituted hydroxamic acid group (—CO—NH(OR⁵⁴⁴)), a carboxamide group (—CO—NH₂), a N-substituted or N,N-disubstituted carboxylic acid amide group, (—CO—NHR⁵⁴⁵; —CO—NR⁵⁴⁶R⁵⁴⁷), an amido group (—HN—CO—R⁵⁴⁸), a sulfonic acid group (—SO₃H), a sulfonamide group (—SO₂—NH₂), a N-substituted or N,N-di substituted sulfonamide group (—SO₂—NHR⁵⁴⁹; —SO₂—NR⁵⁵⁰OR⁵⁵¹), an amidosulfone group (—NH—SO₂—R⁵⁵²), a sulfone group (—SO₂—R⁵⁵³), a phosphoric acid group (—OP(═O)(OH)₂), a phosphoric acid ester group (—OP(═O)(OR⁵⁵⁴)(OR⁵⁵⁵)), a phosphonic acid group (—P(═O)(OH)₂), an phosphonic acid ester group (—P(═O)(OR⁵⁵⁶)(OR⁵⁵⁷)), a halogen atom, a trifluormethyl group (—CF₃), a thiol group (—SH); a thioether group (—S—R⁵⁵⁸), a hydroxy group (—OH); an alkoxy group (—O—R⁵⁵⁹), a tetrazole group, an amino group (—NH₂), or a N-substituted or N,N-disubstituted amino group (—NHR⁵⁶⁰; —NR⁵⁶R⁵⁶²); and

which, independently of each other, can be substituted with one or more substituents, which can be the same or different; and,

wherein optionally, the pairs R⁵⁴⁶/R⁵⁴⁷, R⁵⁵⁰/R⁵⁵¹, R⁵⁵⁴/R⁵⁵⁵, R⁵⁵⁶/R⁵⁵⁷ and R⁵⁶¹/R⁵⁶² independenly of each other, may form a part of a ring; and

wherein the substituents R⁵⁴⁰, R⁵⁴¹, R⁵⁴², R⁵⁴³, R⁵⁴⁴, R⁵⁴⁵, R⁵⁴⁶, R⁵⁴⁷, R⁵⁴⁸, R⁵⁴⁹, R⁵⁵⁰, R⁵⁵¹, R⁵⁵², R⁵⁵³, R⁵⁵⁴, R⁵⁵⁵, R⁵⁵⁶, R⁵⁵⁷, R⁵⁵⁸, R⁵⁵⁹, R⁵⁶⁰, R⁵⁶¹, and R⁵⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;

or wherein the group PM

has the formula (IX) or (IXa)

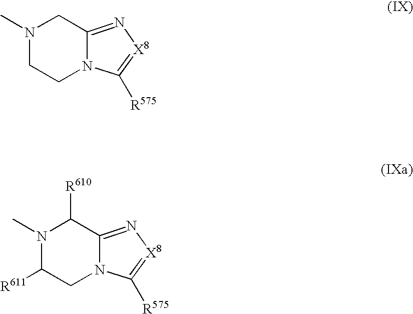

wherein X⁸ is N or CR⁵⁷⁰; and

wherein R⁵⁷⁰, R⁵⁷⁵, R⁶¹⁰ and R⁶¹¹ independently of each other, are

a hydrogen atom (—H), a methyl group (—CH₃), a trifluoromethyl group (—CF₃), an ethyl group (—C₂H₅), a 2,2,2-trifluoroethyl group (—CH₂CF₃), a pentafluoroethyl group (—CF₂CF₃), a phenyl group, (—C₆H₅), a benzyl group (—CH₂—C₆H₅), a benzyloxy group (—OCH₂—C₆H₅), a para-ethyl-phenyl group (—C₆H₄—C₂H₅), a para-fluorophenyl group (—C₆H₄-4-F), a 3,4-difluorophenyl group (—C₆H₃-3,4-F₂), a para-methoxyphenyl group (—C₆H₄-4-OCH₃), a para-trifluoromethoxyphenyl group (—C₆H₄-4-OCF₃), a boronic acid group (—B(OH)₂), a cyano group (—C≡N), a carboxylic acid group (—COOH), or a phosphonic acid ester group (—P(═O)(OR⁵⁹⁶)(OR⁵⁹⁷));

which, independently of each other, can be substituted with one or more substituents, which can be the same or different; and,

wherein optionally, the pairs R⁵⁷⁰/R⁵⁷⁵, if present, as well as the pair R⁵⁹⁶/R⁵⁹⁷, independenly of each other, may form a part of a ring; and

wherein the substituents R⁵⁹⁶ and R⁵⁹⁷, independently of each other are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅ cycloalkyl, cyano, amido, thiol trifluoromethyl, or hydroxy group; and

or wherein the group PM

has the formula (IX)

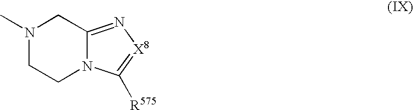

wherein X⁸ is N or CR⁵⁷⁰; and

wherein R⁵⁷⁰ and R⁵⁷⁵, independently of each other, are

(1) hydrogen,

(2) CN,

(3) C₁₋₁₀alkyl, which is linear or branched and which is unsubstituted or substituted with 1-5 halogens or phenyl, which is unsubstituted or substituted with 1-5 substituents independently selected from halogen, CN, OH, R⁶¹², OR⁶¹², NHSO₂R⁶¹², SO₂R⁶¹², SO₂R⁶¹², CO₂H, and CO₂C₁₋₆ alkyl, wherein the CO₂C₁₋₆ alkyl is linear or branched,

(4) phenyl which is unsubstituted or substituted with 1-5 substituents independently selected from halogen, CN, OH, R⁶¹², OR⁶¹², NHSO₂R⁶¹², SO₂R⁶¹², CO₂H, and CO₂C₁₋₆ alkyl, wherein the CO₂C₁₋₆ alkyl is linear or branched, and

(5) a 5- or 6-membered heterocycle which may be saturated or unsaturated comprising 1-4 heteroatoms independently selected from N, S, and O, the heterocycle being unsubstituted or substituted with 1-3 substituents independently selected from oxo, OH, halogen, C₁₋₆ alkyl, and OC₁₋₆ alkyl, wherein C₁₋₆alkyl and C₁₋₆ alkoxy are linear or branched and optionally substituted with 1-5 halogens, and

wherein R⁶¹² is C₁₋₆ alkyl, which is linear or branched and which is unsubstituted or substituted with 1-5 groups independently selected from halogen, CO₂H, and CO₂C₁₋₆ alkyl, wherein the CO₂C₁₋₆ alkyl is linear or branched.

or wherein the group PM

has the formula (IXa)

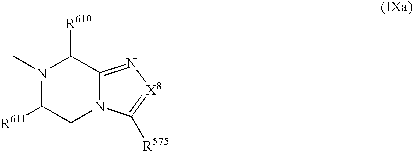

wherein X⁸ is N or CR⁵⁷⁰; and

wherein R⁵⁷⁰ and R⁵⁷⁵ independently of each other, are

(6) hydrogen,

(7) CN,

(8) C₁₋₁₀ alkyl, which is linear or branched and which is unsubstituted or substituted with 1-5 halogens or phenyl, which is unsubstituted or substituted with 1-5 substituents independently selected from halogen, CN, OH, R⁶¹², OR⁶¹², NHSO₂R⁶¹², SO₂R⁶¹², CO₂H, and CO₂C₁₋₆ alkyl, wherein the CO₂C₁₋₆ alkyl is linear or branched,

(9) phenyl which is unsubstituted or substituted with 1-5 substituents independently selected from halogen, CN, OH, R⁶¹², OR⁶¹², NHSO₂R⁶¹², SO₂R⁶¹², CO₂H, and CO₂C₁₋₆ alkyl, wherein the CO₂C₁₋₆ alkyl is linear or branched, and

(10)a 5- or 6-membered heterocycle which may be saturated or unsaturated comprising 14 heteroatoms independently selected from N, S, and O, the heterocycle being unsubstituted or substituted with 1-3 substituents independently selected from oxo, OH, halogen, C₁₋₆ alkyl, and OC₁₋₆ alkyl, wherein C₁₋₆ alkyl and C₁₋₆ alkoxy are linear or branched and optionally substituted with 1-5 halogens, and

wherein R⁶¹² is C₁₋₆ alkyl, which is linear or branched and which is unsubstituted or substituted with 1-5 groups independently selected from halogen, CO₂H, and CO₂C₁₋₆ alkyl, wherein the CO₂C₁₋₆ alkyl is linear or branched, and

wherein R⁶¹⁰ and R⁶¹¹ are each independently selected from the group consisting of

(1) hydrogen,

(2) C₁₋₁₀ alkyl, which is linear or branched and which is unsubstituted or substituted with one or more substituents selected from:

(a) halogen,

(b) hydroxy,

(c) phenyl, wherein the phenyl is unsubstituted or substituted with 1-5 substituents independently selected from halogen, OH, C₁₋₆ alkyl, and C₁₋₆ alkoxy, wherein the C₁₋₆ alkyl, and C₁₋₆ alkoxy are linear or branched and optionally substituted with 1-5 halogens,

(d) naphthyl, wherein the naphthyl is optionally substituted with 1-5 substituents independently selected from halogen, CN, OH, C₁₋₆ alkyl, and C₁₋₆ alkoxy, wherein the C₁₋₆ alkyl, and C₁₋₆ alkoxy are linear or branched and optionally substituted with 1-5 halogens,

(e) CO₂H,

(f) CO₂C₁₋₆ alkyl,

(g) CONR⁶¹³R⁶¹⁴, wherein R⁶¹³ and R⁶¹⁴ are independently selected from the group consisting of hydrogen, tetrazolyl, phenyl, C₃₋₆ cycloalkyl and C₁₋₆ alkyl, wherein the C₁₋₆ alkyl is linear or branched and is optionally substituted with 1-6 substituents independently selected from 0-5 halogen and 0-1 phenyl, wherein the phenyl or the C₃₋₆ cycloalkyl beeing R⁶¹³ and R⁶¹⁴ or the optional phenyl substituent on the C₁₋₆ alkyl are optionally substituted with 1-5 substituents independently selected from halogen, OH, C₁₋₆ alkyl, and OC₁₋₆ alkyl, said C₁₋₆ alkyl and OC₁₋₆alkyl being linear or branched and optionally substituted with 1-5 halogens, or wherein R⁶¹³ and R⁶¹⁴ are optionally joined to form a ring selected from pyrrolidine, piperidine or morpholine,

(3) CN,

(4) phenyl, wherein the phenyl is unsubstituted or substituted with 1-5 substituents independently selected from C₁₋₆ alkyl, and C₁₋₆ alkoxy, hydroxy and halogen, wherein the C₁₋₆ alkyl, and C₁₋₆ alkoxy are linear or branched and optionally substituted with 1-5 halogens,

(5) naphthyl, wherein the naphthyl is unsubstituted or substituted with 1-5 substituents independently selected from halogen, OH, C₁₋₆ alkyl, and C₁₋₆ alkoxy, wherein the C₁₋₆ alkyl, and C₁₋₆ alkoxy are linear or branched and optionally substituted with 1-5 halogens,

(6) CO₂H,

(7) CO₂C₁₋₆ alkyl,

(8) CONR⁶¹³R⁶¹⁴, and

(9) C₃₋₆ cycloalkyl, which is optionally substituted with 1-5 substituents independently selected from halogen, OH, C₁₋₆ alkyl, and C₁₋₆ alkoxy, wherein the C₁₋₆ alkyl, and C₁₋₆alkoxy are linear or branched and optionally substituted with 1-5 halogen, with the proviso that one of R⁶¹⁰ and R⁶¹¹ is other than hydrogen.

or wherein the group PM

has the formula (X)

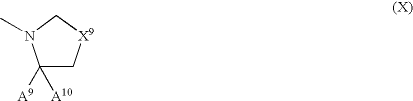

wherein the groups X⁹ is CR⁹⁰⁰R⁹⁰¹, S, SO, SO₂or NR⁹⁰²

wherein R⁹⁰⁰, R⁹⁰¹ and R⁹⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR⁹¹⁰R⁹¹¹.

wherein A⁹ and A¹⁰ are, independently of each other, selected from hydrogen, cyano, —C(═O)NR⁹¹²R⁹¹³ or C₁, C₂, C₃, C₄, C₅or C₆alkyl, which is linear or branched and is optionally substituted with 1, 2, 3, 4, or 5 halogens;

wherein

R⁹¹⁰ and R⁹¹², are, independently of each other, selected from hydrogen, or C₁, C₂, C₃, C₄, C₅or C₆alkyl, which is linear or branched and is optionally substituted with 1, 2, 3, 4, or 5 halogens; and

R⁹¹¹ and R⁹¹³, are, independently of each other, selected from the group consisting of

(1) phenyl, which is optionally substituted with 1, 2, 3, 4, or 5, substituents independently selected from halogen and R⁹²⁰;

(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of

(a) hydroxy,

(b) —COOH,

(c) —COO(C₁, C₂, C₃, C₄, C₅or C₆alkyl), i.e. ester,

(d) phenyl,

(e) naphthyl,

(f) C₃, C₄, C₅or C₆cycloalkyl,

(g) a 5- or 6 membered heterocycle which may be saturated or unsaturated comprising 1, 2, 3, or 4 heteroatoms independently selected from nitrogen, oxygen or sulfur;

(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;

wherein said C₃, C₄, C₅or C₆cycloalkyl, phenyl, naphthyl, are optionally substituted with 1, 2, 3, 4, or 5 substituents independently selected from halogen and R⁹²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R⁹²⁰; and

(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;

wherein R⁹²⁰ is selected from the group consisting of:

(1) hydroxy;

(2) cyano;

(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC I, —OC₂, —OC₃, —OC₄, —OC₅or —OC₆alkyl substituents being linear or branched and optionally substituted with 1, 2, 3, 4, or 5 halogens;

(4) C₁, C₂, C₃, C₄, C₅, C₆, C₇, C₈, C₉ or C₁₀ alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from

(a) hydroxy;

(b) —COOH;

(c) —COO(C₁, C₂, C₃, C₄, C₅or C₆alkyl) i.e. ester, which may linear or branched and is optionally substituted with 1, 2, 3, 4, or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR⁹²⁵R⁹²⁵;

(g) —SO₂NR⁹²⁵R⁹²⁵

(h) —NR⁹²⁵—C(═O)R⁹²⁵

(i) —NR⁹²⁵—C(═O)NR⁹²⁵R⁹²⁵;

o) —NR⁹²⁵COOR⁹³⁰

(k) —O—CO—R⁹³⁰

(l) —O—CO—NR⁹²⁵R⁹²¹;

(m) —NR⁹²⁵SO₂R⁹³⁰;

(n) NR⁹²⁵R⁹²⁵;

(o) phenyl which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(5) OC₁, OC₂, OC₃, OC₄, OC₅, OC₆, OC₇, OC₈, OC₉or OC₁₀alkyl, which is linear or branched and is optionally substituted with 0, 1, 2, 3, 4, or 5 halogen atoms and 0, 1, or 2 substitutents selected from

(a) hydroxy;

(b) —COOH;

(c) —COO(C₁, C₂, C₃, C₄, C₅or C₆alkyl) i.e. ester, which may be linear or branched and is optionally substituted with 1, 2, 3, 4 or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, Cs or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR⁹²⁵, R⁹²⁵;

(g) —SO₂NR⁹²⁵R⁹²⁵;

(h) —NR⁹²⁵—C(═O)R⁹²⁵

(i) —NR⁹²⁵C(═O)NR⁹²⁵R⁹²⁵

(j) —NR⁹²⁵ COOR⁹³⁰

(k) —O—CO—R⁹³⁰

(l) —O—CO—NR⁹²⁵R⁹²¹;

(m) NR⁹²⁵ SO₂R⁹³⁰;

(n) NR⁹²⁵R⁹²⁵;

(o) phenyl, which is optionally substituted with 1, 2, 3, 4, or 5 groups independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(6) —COOH;

(7) —COO(C₁, C₂, C₃, C₄, C₅or C₆alkyl) i.e. ester, which may be linear or branched and is optionally substituted with 1, 2, 3, 4, 5 halogens;

(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.

(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(10)—CONR⁹²⁵R⁹²⁵;

(11) —SO₂NR⁹²⁵R⁹²⁵;

(12) —NR⁹²⁵—C(═O)R⁹²⁵

(13) —NR⁹²⁵—C(═O)NR⁹²⁵R⁹²⁵;

(14) —NR⁹²⁵COOR⁹³⁰

(15) —O—CO—R⁹³⁰

(16) —O—CO—NR⁹²⁵R⁹²⁵;

(17) —NR⁹²⁵ SO₂R⁹³⁰;

(18)—NR⁹²⁵R⁹²⁵;

(19) phenyl, which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;

wherein R⁹³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.

wherein R⁹²⁵ is selected from R⁹³⁰ and hydrogen.

wherein the group PM

has the formula (XI)

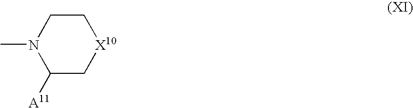

wherein the groups X¹⁰ is CR¹⁰⁰⁰R¹⁰⁰¹, S, SO, SO₂or NR¹⁰⁰²

wherein R¹⁰⁰⁰, R¹⁰⁰¹ and R¹⁰⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR¹⁰¹⁰R¹⁰¹¹.

and A¹¹ is selected from

hydrogen, cyano, —C(═O)NR¹⁰¹²R¹⁰¹³, or C₁, C₂, C₃, C₄, C₅or C₆alkyl, which is linear or branched and is optionally substituted with 1, 2, 3, 4, or 5 halogens;

wherein

R¹⁰¹⁰ and R¹⁰¹², are, independently of each other, selected from hydrogen, or C₁, C₂, C₃, C₄, C₅or C₆alkyl, which is linear or branched and is optionally substituted with 1, 2, 3, 4, or 5 halogens; and

R¹⁰¹¹ and R¹⁰¹³, are, independently of each other, selected from the group consisting of

(1) phenyl, which is optionally substituted with 1, 2, 3, 4, or 5, substituents independently selected from halogen and R¹⁰²⁰;

(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of

(a) hydroxy,

(b) —COOH,

(c) —COO(C₁, C₂, C₃, C₄, C₅or C₆alkyl), i.e. ester,

(d) phenyl,

(e) naphthyl,

(f) C₃, C₄, C₅or C₆cycloalkyl,

(g) a 5- or 6 membered htereocycle which may be saturated or unsaturated comprising 1, 2, 3, or 4 heteroatoms independently selected from nitrogen, oxygen or sulfur;

(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;

wherein said C₃, C₄, C₅or C₆cycloalkyl, phenyl, naphthyl, are optionally substituted with 1, 2, 3, 4, or 5 substituents independently selected from halogen and R¹⁰²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R¹⁰²⁰; and

(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;

wherein R¹⁰²⁰ is selected from the group consisting of:

(1) hydroxy;

(2) cyano;

(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;

(4) C₁, C₂, C₃, C₄, C₅, C₆, C₇, C₈, C₉or C₁₀alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from

(a) hydroxy;

(b) —COOH;

(c) —COO(C₁, C₂, C₃, C₄, C₅or C₆alkyl) i.e. ester, which may linear or branched and is optionally substituted with 1, 2, 3, 4, or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR¹⁰²⁵R¹⁰²⁵;

(g) —SO₂NR¹⁰²⁵R¹²⁵;

(h) —NR¹⁰²⁵—C(═O)R¹⁰²⁵

(i) —NR¹⁰²⁵—C(═O)NR¹⁰²⁵, R¹²⁵

(j) —NR¹⁰²⁵COOR¹⁰³⁰

(k) —O—CO—R¹⁰³⁰

(l) —O—CO—NR¹⁰²⁵R¹⁰²⁵;

(m) —NR¹⁰²⁵SO₂R¹⁰³⁰;

(n) _NR¹⁰²⁵R¹⁰²⁵;

(o) phenyl which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(5) OC₁, OC₂, OC₃, OC₄, OC₅, OC₆, OC₇, OC₈, OC₉or OC₁₀alkyl, which is linear or branched and is optionally substituted with 0, 1, 2, 3, 4, or 5 halogen atoms and 0, 1, or 2 substitutents selected from

(a) hydroxy;

(b) —COOH;

(c) —COO(C₁, C₂, C₃, C₄, C₅or C₆alkyl) i.e. ester, which may be linear or branched and is optionally substituted with 1, 2, 3, 4 or 5 halogens;

(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;

(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;

(f) —CONR¹⁰²⁵R¹⁰²⁵;

(g) —SO₂NR¹⁰²⁵R¹⁰²⁵

(h) —NR¹⁰²⁵—C(═O)R¹⁰²¹

(i) —NR¹⁰²⁵-c(═O)NR¹⁰²⁵, R¹²⁵

(j) —NR¹⁰²⁵COOR¹⁰³⁰

(k) —O—CO—R¹⁰³⁰

(l) —O—CO—NR¹⁰²⁵R¹⁰²⁵;

(m) —NR¹⁰²⁵SO₂R¹⁰³⁰;

(n) —NR¹⁰²⁵R¹⁰²⁵;

(o) phenyl, which is optionally substituted with 1, 2, 3, 4, or 5 groups independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and

(p) C₃, C₄C₅or C₆cycloalkyl, which is optionally substituted with 1, 2, 3, 4, 5, or 6 halogens;

(6) —COOH;

(7) —COO(C₁, C₂, C₃, C₄, C₅or C₆alkyl) i.e. ester, which may be linear or branched and is optionally substituted with 1, 2, 3, 4, 5 halogens;

(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.

(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, 5-OC₅or —OC₆alkyl being linear or branched and optionally substituted with 1, 2, 3, 4, or 5 halogens;

(10) —CONR¹⁰²⁵R¹⁰²⁵;

(11)—SO₂NR¹⁰²⁵R¹⁰²⁵;

(12) —NR¹⁰²⁵—C(═O)R¹⁰²⁵

(13)—NR¹⁰²⁵—C(═O)NR¹²⁵R¹⁰²⁵

(14)—NR⁹²⁵COOR¹⁰³⁰

(15)—O—CO—R¹⁰³⁰

(16) —O—CO—NR¹⁰²⁵R¹⁰²⁵;

(17) NR¹⁰²⁵SO₂R¹³⁰;

(18) NR¹⁰²⁵R¹⁰²⁵;

(19) phenyl, which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;

wherein R¹⁰³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is 25 linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, 30-OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens,

wherein R¹⁰²⁵ is selected from R¹⁰³⁰ and hydrogen.

or wherein the group PM

has the formula (XII)

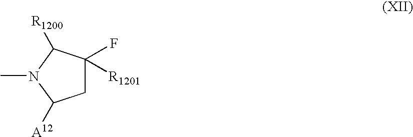

wherein the groups R¹²⁰¹ is hydrogen or fluoro.

wherein R¹²⁰⁰ und A¹² is selected from hydrogen and cyano, and the other is hydrogen.

or wherein the group PM

has the formula XIII:

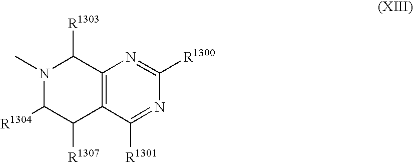

wherein:

R¹³⁰⁰ is selected from the group consisting of:

(1) hydrogen,

(2) CN,

(3) C₁₋₁₀alkyl, which is linear or branched which is unsubstituted or substituted with:

a) halogen, or

b) phenyl, which is unsubstituted or substituted with 1-5 substituents independently selected from halogen, CN, OH, R¹³⁰², OR¹³⁰², NHSO₂R¹³⁰², N(C₁₋₆alkyl)SO₂R¹³⁰², SO₂R¹³⁰², SO₂NR¹³⁰⁵R¹³⁰⁶, NR¹³⁰⁵R¹³⁰⁶ CONR¹³⁰⁵R¹³⁰⁶, CO₂H, and CO₂C₁₋₆alkyl, wherein the C₁₋₆alkyl is linear or branched,

(4) phenyl which is unsubstituted or substituted with 1-5 substituents independently selected from halogen, CN, OH, R¹³⁰², OR¹³⁰², NHSO₂R¹³⁰², N(C₁₋₆alkyl)SO₂R^{1302, SO}₂R¹³⁰², SO₂NR¹³⁰⁵R¹³⁰⁶, NR¹³⁰⁵R¹³⁰⁶, CONR¹³⁰⁵R¹³⁰⁶ CO₂H, and CO₂C₁₋₆alkyl, wherein the C₁₋₆alkyl is linear or branched,

(5) a 5- or 6-membered heterocyclic which may be saturated or unsaturated comprising 1-4 heteroatoms independently selected from N, S and O, the heterocycle being unsubstituted 20 or substituted with 1-3 substituents independently selected from oxo, halogen, NO₂, CN, OH, R¹³⁰², OR¹³⁰², NHSO₂R¹³⁰², N(C₁₋₆alkyl)SO₂R¹³⁰², SO₂R¹³⁰², SO₂NR¹³⁰⁵R¹³⁰⁶ NR¹³⁰⁵R¹³⁰⁶, CONR¹³⁰⁵R¹³⁰⁶, CO₂H, and CO₂C₁₋₆alkyl, wherein the C₁₋₆alkyl is linear or branched,

(6) C₃₋₆cycloalkyl, which is optionally substituted with 1-5 substituents independently 25 selected from halogen, OH, C₁₋₆alkyl, and OC₁₋₆alkyl, wherein the C₁₋₆alkyl and OC₁₋₆ alkyl are linear or branched and optionally substituted with 1-5 halogens,

(7) OH,

(8) OR³⁰² and

(9) NR¹³⁰⁵R¹³⁰⁶;

R¹³⁰¹ is hydrogen;

R¹³⁰² is C₁₋₆alkyl, which is linear or branched and which is unsubstituted or substituted with 1-5 groups independently selected from halogen, CO₂H, and CO₂C₁₋₆alkyl, wherein the C₁₋₆alkyl is linear or branched;

R¹³⁰³ is hydrogen;

R¹³⁰⁵ and R¹³⁰⁶ are independently selected from the group consisting of:

(1) hydrogen,

(2) phenyl, which is unsubstituted or substituted with substituents independently selected from halogen, OH, C₁₋₆alkyl, and OC₁₋₆alkyl, wherein the C₁₋₆alkyl is linear or branched and optionally substituted with 1-5 halogens

(3) C₃-cycloalkyl, which is unsubstituted or substituted with 1-5 substituents independently selected from C₁₋₆alkyl, and OC₁₋₆alkyl, wherein the C₁₋₆alkyl is linear or branched and optionally substituted with 1-5 halogens

(4) C₁₋₆alkyl, which is linear or branched and which is unsubstituted or substituted with:

a) halogen, or

b) phenyl, which is unsubstituted or substituted with 1-5 substituents independently selected from halogen, OH, C₁₋₆alkyl, and OC₁₋₆alkyl, wherein the C₁₋₆alkyl is linear or branched and optionally substituted with 1-5 halogens,

or wherein R¹³⁰⁵ and R¹³⁰⁶ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆alkyl, and C₁₋₆alkoxy, wherein alkyl and alkoxy are unsubstituted with one to five halogens;

R¹³⁰⁴ and R¹³⁰⁷ are hydrogen;

or wherein the group PM

has the formula XIV:

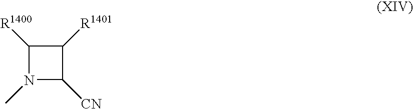

wherein R¹⁴⁰⁰ is H and R¹⁴⁰¹ is hydrogen atom (—H); or fluoro, or cyano.

Synthesis of the Compounds of the Present Invention

The compounds of formula (I) according to the present invention can be obtained by the general method, characterized in that the amino acid amide of the general formula

A—B

is synthesized, wherein

A is NR¹R²—C(=EWG1)—(CR³R⁴)ₙ—CR⁵R⁶—CR⁷R⁸—CR⁹(NR¹⁰R¹¹)—C(=EWG2) as defined above, and

B is a proline mimetic (PM) as defined above, and

wherein their production is performed by starting from X-A-Y or X-A(Z)-Y (in case of trifunctional amino acids for A) by substitution with B, wherein A and B are defined as described above, X stands for an α-amino-protecting group commonly used in peptide chemistry, preferably the t-butyloxycarbonyl residue, Z represents a common side chain-protecting group, preferably of the t-butyl-type (t-butyloxycarbonyl, t-butyl ester, O- or S-t-butyl) depending on the structure of the trifunctional amino acid, and Y means hydroxy, active ester, preferably pentafluorophenyl or N-hydroxsuccinimide ester, according the method common in the peptide chemistry for attachment of the amide bond, desirably via the anhydride mixture technique or the active ester method, then the protecting groups used for X and Z are removed with the deblocking method common in the peptide chemistry for the above-mentioned of the t-butyl type through acidolysis, and if necessary, the products are purified through re-crystallization or through column chromatography on Sephadex G10 or weakly acidic ion exchange resin.

Specific synthetic routes and synthetic schemes for the respective proline mimetics of the present invention are well known in the state of the art. References which disclose these synthetic routes and synthetic schemes of compounds which comprise the proline mimetics of the present invention, are listed in table 2. These references are incorporated herein in their entirety and are part of the present invention with regard to the synthesis of the compounds of the present invention comprising the respective proline mimetics.

TABLE 2
References disclosing the synthetic routes and synthesis schemes of proline
mimetics according to the present invention

| Reference for synthetic route and | |
|---|---|
| synthesis schemes | Proline mimetic (PM) |
| WO 01/34594 A1, pp. 21-22, International Publication Date: May 17, 2001 | 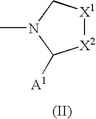 |
| WO 01/34594 A1, pp. 48-49, International Publication Date: May 17, 2001 | 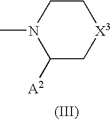 |
| WO 01/34594 A1, p. 57, International Publication Date: May 17, 2001 | 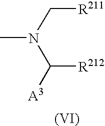 |
| WO 01/55105 A1, pp. 17-18, International Publication Date: Aug. 2, 2001 | 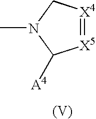 |
| 1. WO 02/38541, especially engl. version EP 1333025A1 thereof, pp. 8-14, Date of Publication: Aug. 6, 2003 2. when A⁵ = H and R³⁷¹, R³⁷⁵ and R³⁷⁶ =WO 03/101449A2, pp. 6-10, International Publication Date: Dec. 11, 2003 | 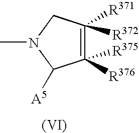 |
| WO 01/68603A2, pp. 8-11, International. Publication Date: Sep. 20, 2001 | 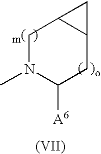 |
| WO 02/083128A1, pp.7-10, International. Publication Date: Oct. 24, 2002 | 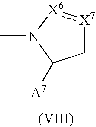 |
| 1. for PM (IX): WO 03/004498A1, pp. 24-28, International. Publication Date: Jan. 16, 2003 2. for PM (IXa): WO 03/082817A2, pp. 29-37, International. Publication Date: Oct. 9, 2003 | 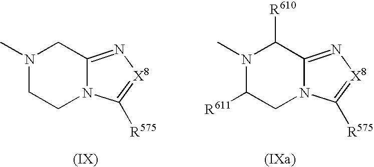 |
| WO 03/000180A2, pp 26-35, International Publication Date: Jan. 3, 2003 | 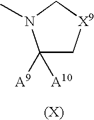 |
| WO 03/000181A2, pp. 25-32, International Publication Date: Jan. 3, 2003 | 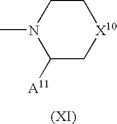 |
| WO 03/00250A1, pp. 11-14, International Publication Date: Jan. 3, 2003 | 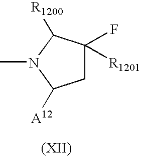 |
| WO 04/007468A1, pp. 28-39, International Publication Date: Jan. 22, 2004 | 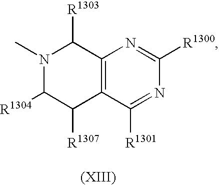 |
| WO 04/007446A1, pp. 12-16, International Publication Date: Jan. 22, 2004 | 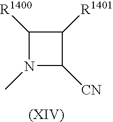 |
| WO 04/026822A2, pp. 32-40, International Publication Date: Apr. 1, 2004 | 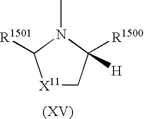 |

A further preferred embodiment of the present invention comprises the compound of the general formula (I) according to any one of the embodiments of the present invention

in combination with acarbose, or

in combination with metformin; or

in combination with acarbose and metformin.

In a further preferred embodiment the DP IV inihibitors of the general formula (I) of the present invention, optionally in combination with QC inhibitor, can be used in combination with

(a) other DP IV inhibitors

(b) insulin sensitizers selected from the group consisting of

(i) PPAR agonists,

(ii) biguanides, and

(iii) protein tyrosin phosphatase-1B (PTP-1B) inhibitors;

(c) insulin and insulin mimetics;

(d) sulfonylureas and other insulin secretagogues;

(e) α-glucosidase inhibitors;

(f) glucagon receptor agonists;

(g) GLP-1; GLP-1 mimetics, e.g. NN-2211 (liraglutide from Novo Nordisk), and GLP-1 receptor agonists;

(h) GLP-2; GLP-2 mimetics, e.g. ALX-0600 (teduglutide from NPS Allelix Corp.) and GLP-2 receptor agonists;

(i) exendin-4 and exendin-4 mimetics, e.g. exenatide (AC-2993, synthetic exendin-4 from Amylin/Eli Lilly);

(j) GIP, GIP mimetics, and GIP receptor agonists;

(k) PACAP, PACAP mimetics, and PACAP receptor 3 agonists;

(l) choletserol lowering agents selected from the group consisting of

(i) HMG-CoA reductase inhibitors,

(ii) sequestrants,

(iii) nicotinyl alkohol, nicotinic acid and salts thereof,

(iv) PPARoc agonists,

(v) PPARo/y dual agonists,

(vi) inhibitors of cholesterol absorption,

(vii) acyl CoA:cholesterol acyltransferase inhibitors, and

(viii) antioxidants;

(m) PPAR⁸ agonists;

(n) antiobesity compounds;

(o) an ileal bile acid transporter inhibitor; and

(p) anti-inflammatory agents.

A further preferred embodiment of the present invention comprises the compound of the general formula (I) according to any one of the embodiments of the present invention mentioned above

in combination with a gene therapeutic expression system for GLP-1 comprising a viral vector comprising

(a) a polynucleotide sequence encoding GLP-1 (gluacogen like peptide-1); and

(b) a polynucleotide sequence encoding a signal sequence upstream of (a); and

(c) a polyadenylation signal downstream of (a); and

(d) a polynucleotide sequence encoding a proteolytic cleavage site located between the polynucleotide sequence encoding GLP-1 and the polynucleotide sequence encoding the signal sequence; and

(e) wherein the expression of GLP-1 underlies a constitutive promoter or is controlled by a regulatable promotor;

(f) wherein, optionally, the viral vector comprises a polynucleotide sequence encoding GIP (glucose dependent insulinotropic peptide);

(g) wherein, optionally, the viral vector is encompassed by a mammalian cell.

and/or

in combination with a gene therapeutic expression system for GIP comprising a viral vector comprising

(a) a polynucleotide sequence encoding GIP (glucose dependent insulinotropic peptide); and

(b) a polynucleotide sequence encoding a signal sequence upstream of (a); and

(c) a polyadenylation signal downstream of (a); and

(d) a polynucleotide sequence encoding a proteolytic cleavage site located between the polynucleotide sequence encoding GIP and the polynucleotide sequence encoding the signal sequence; and

(e) wherein the expression of GIP underlies a constitutive promoter or is controlled by a regulatable promotor;

(f) wherein, optionally, the viral vector comprises a polynucleotide sequence encoding GLP-1 (glucagon like peptide 1);

(g) wherein, optionally, the viral vector is encompassed by a mammalian cell.

A further preferred embodiment of the present invention comprises the compound of the general formula (I) in combination with a gene therapeutic expression system for GLP-1 and/or GIP according to any one of the embodiments of the present invention mentioned above wherein

the signal sequence upstream of the gene of interest (GLP-1; GIP) is the murine immunoglobulin K signal sequence or the glia monster exendin signal sequence; and/or

the polyadenylation signal downstream of the gene of interest (GLP-1; GIP) is derived from simian viraus 40 (SV 40); and/or

the proteolytic cleavage site is cleaved by furin preotease; and/or

the gene delivery vector for expression the gene of interest is an adenoviral, retroviral, leniviral, adeno associated viral vector; and/or

the constitutive promoter is a cytomegalovirus (CMV) promotor, or a Rous sarcoma long-terminal repeat (LTR) sequence, and the SV 40 early gene gene promoter; and the inducible promoter is the Tet-On™/Tet-Off™ system available from Clontech; and/or

the mammalian cell is a primate or rodent cell, preferably a human cell, more preferably a human hepatocyte.

A further preferred embodiment of the present invention comprises the compound of the general formula (I) in combination with a glutaminyl cyclase (QC) inhibitor, and, additionally, a gene therapeutic expression system for GLP-1 and/or GIP according to any one of the embodiments of the present invention mentioned above.

In a preferred embodiment, the compound of the general formula (I) according to the present invention is used in the form of a pharmaceutical composition comprising a composition according to any one the embodiments mentioned, and optionally a pharmaceutical acceptable diluent and/or carrier.

In a preferred embodiment, the compound of the general formula (I) according to the present invention is used in the form of a composition or a pharmaceutical composition according to any one of the preceding embodiments for the preparation of a medicament for the inhibition of dipeptidyl peptidase IV.

In a preferred embodiment, the compound of the general formula (I) according to the present invention is used in the form of a composition or a pharmaceutical composition according to any one of the preceding embodiments for the preparation of a medicament for the treatment of disorders related to the inhibition of dipeptidyl peptidase IV. Examples for disorders related to the inhibition of DP IV which can be treated by DP IV inhibitors according to the present invention are listed under item “Indications”.

In a more preferred embodiment, the compound of the general formula (I) according to the present invention, which is an inhibitor of dipeptidyl peptidase (DPIV), may be used in combination with an inhibitor of glutaminyl cyclase (QC).

In a preferred embodiment, the compound of the general formula (I) according to the present invention may be used in the form of a composition or a pharmaceutical composition according to any one of the preceding embodiments for the preparation of a medicament for the treatment of diseases of mammals that can be treated by modulation of DPIV- and, optionally, QC activity, in a mammal, especially for the treatment of metabolic diseases in humans.

Inhibitors of glutaminyl cyclase are, e.g. compounds having the general formula 1, including the pharmaceutically acceptable salts and including all stereoisomers thereof:

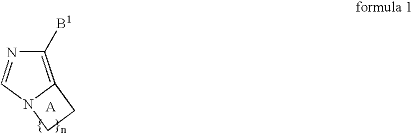

wherein n is 1, 2, 3 or 4, preferably 2 and 3, most preferred 2, and A can be any saturated or unsaturated heterocycle and wherein B¹ is H or a branched or unbranched alkyl chain, a branched or unbranched alkenyl chain, a branched or unbranched alkynyl chain, carbocyclic, aryl, heteroaryl, heterocyclic, aza-amino acid, amino acid or a mimetic thereof, aza-peptide, peptide or a mimetic thereof; all of the above residues optionally being substituted.

Further inhibitors of glutaminyl cyclase are, e.g. compounds which can be described generally by the formula 2, including the pharmaceutically acceptable salts and including all stereoisomers thereof:

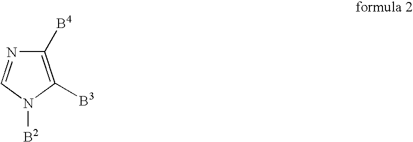

wherein B², B³ and B are independently H or a branched or unbranched alkyl chain, a branched or unbranched alkenyl chain, a branched or unbranched alkynyl chain, carbocyclic, aryl, heteroaryl, heterocyclic, aza-amino acid, amino acid or a mimetic thereof, aza-peptide, peptide or a mimetic thereof; all of the above residues optionally being substituted.

Furthermore, inhibitors of glutaminyl cyclase are compounds which can be described generally by the formula 3, including the pharmaceutically acceptable salts and including all stereoisomers thereof:

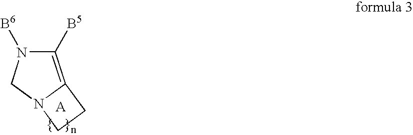

wherein n is 1, 2, 3 or 4, preferably 2 and 3, most preferred 2, and A can be any saturated or unsaturated heterocycle and wherein B⁵ and B⁶ are independently H or a branched or unbranched alkyl chain, a branched or unbranched alkenyl chain, a branched or unbranched alkynyl chain, carbocyclic, aryl, heteroaryl, heterocyclic, aza-amino acid, amino acid or a mimetic thereof, aza-peptide, peptide or a mimetic thereof; all of the above residues optionally being substituted.

Furthermore, inhibitors of glutaminyl cyclase are compounds which can be described generally by the formula 4 and the pharmaceutically acceptable salts thereof, including all stereoisomers:

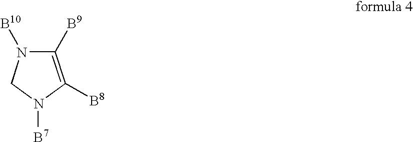

wherein B⁷, B⁸, B⁹ and B¹⁰ are independently H or a branched or unbranched alkyl chain, a branched or unbranched alkenyl chain, a branched or unbranched alkynyl chain, carbocyclic, aryl, heteroaryl, heterocyclic, aza-amino acid, amino acid or a mimetic thereof, aza-peptide, peptide or a mimetic thereof; all of the above residues optionally being substituted.

Furthermore, inhibitors of glutaminyl cyclase are compounds which can be described generally by the formula 5 and the pharmaceutically acceptable salts thereof, including all stereoisomers:

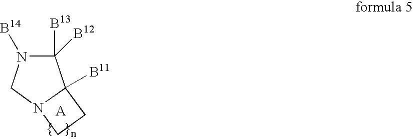

wherein n is 1, 2, 3 or 4, preferably 2 and 3, especially 2, and A can be any saturated or unsaturated heterocycle and wherein B¹¹, B¹¹, B¹³ and B¹⁴ are independently H or a branched or unbranched alkyl chain, a branched or unbranched alkenyl chain, a branched or unbranched alkynyl chain, carbocyclic, aryl, heteroaryl, heterocyclic, aza-amino acid, amino acid or a mimetic thereof, aza-peptide, peptide or a mimetic thereof; all of the above residues optionally being substituted.

Furthermore, inhibitors of glutaminyl cyclase are compounds which can be described generally by the formula 6 and the pharmaceutically acceptable salts thereof, including all stereoisomers:

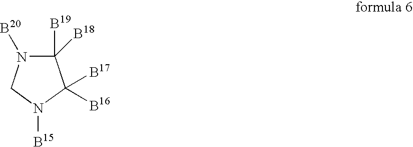

wherein B¹⁵, B¹⁶, B¹⁷, B¹⁸, B¹⁹ and B²⁰ are independently H or a branched or unbranched alkyl chain, a branched or unbranched alkenyl chain, a branched or unbranched alkynyl chain, carbocyclic, aryl, heteroaryl, heterocyclic, aza-amino acid, amino acid or a mimetic thereof, aza-peptide, peptide or a mimetic thereof; all of the above residues optionally being substituted.

In addition, inhibitors of glutaminyl cyclase are compounds which can be described generally by the formula 7, including the pharmaceutically acceptable salts and including all stereoisomers thereof:

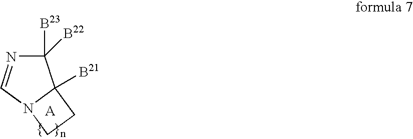

wherein n is 1, 2, 3 or 4, preferably 2 and 3, especially 2, and A can be any saturated or unsaturated heterocycle and wherein B², B²² and B²³ are independently H or a branched or unbranched alkyl chain, a branched or unbranched alkenyl chain, a branched or unbranched alkynyl chain, carbocyclic, aryl, heteroaryl, heterocyclic, aza-amino acid, amino acid or a mimetic thereof, aza-peptide, peptide or a mimetic thereof; all of the above residues optionally being substituted.

Furthermore, inhibitors of glutaminyl cyclase are compounds which can be described generally by the formula 8, including the pharmaceutically acceptable salts and including all stereoisomers thereof:

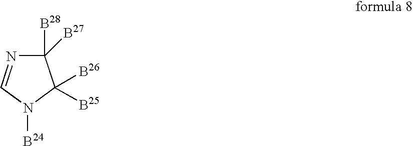

wherein B²⁴, B²⁵, B²⁶, B²⁷ and B²⁸ are independently H or a branched or unbranched alkyl chain, a branched or unbranched alkenyl chain, a branched or unbranched alkynyl chain, carbocyclic, aryl, heteroaryl, heterocyclic, aza-amino acid, amino acid or a mimetic thereof, aza-peptide, peptide or a mimetic thereof; all of the above residues optionally being substituted.

Furthermore, inhibitors of glutaminyl cyclase are compounds which can be described generally by the formula 9 or the pharmaceutically acceptable salts thereof, including all stereoisomers:

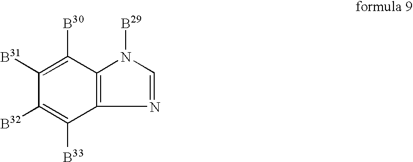

wherein B²⁹, B³⁰, B³¹, B³² and B³³ are independently H or a branched or unbranched alkyl chain, a branched or unbranched alkenyl chain, a branched or unbranched alkynyl chain, carbocyclic, aryl, heteroaryl, heterocyclic, aza-amino acid, amino acid or a mimetic thereof, aza-peptide, peptide or a mimetic thereof; all of the above residues optionally being substituted.

Examples of inhibitors of glutarninyl cyclase are imidazole and its derivatives and histidine and its derivatives. Structures and Ki-values for inhibition of glutaminyl cyclase activity are shown in tables 3 and 4. The results are described in detail in example 9.

TABLE 3
Inhibitory constants of imidazole derivatives in the human QC
catalyzed reaction. Determinations were performed at 30° C.in 0.05 M
Tris-HCl pH 8.0, containing 5 mM EDTA.

| Compound | Kᵢ-value (mM) | Structure |
|---|---|---|
| core structures | | |
| imidazole | 0.103 ± 0.004 | |
| benzimidazole | 0.138 ± 0.005 | |
| N-1 DERIVATIVES | | |
| 1-benzylimidazole | 0.0071 ± 0.0003 | |
| 1-methylimidazole | 0.030 ± 0.001 | |
| 1-vinylimidazole | 0.049 ± 0.002 | |
| oxalic acid dilmidazolidide | 0.078 ± 0.002 | |
| N-acetylimidazole | 0.107 ± 0.003 | |
| N-(trimethylsilyl)-imidazole | 0.167 ± 0.007 | |
| N-benzoylimidazole | 0.174 ± 0.007 | |
| 1 -(2-oxo-2-phenyl-ethyl)- | 0.184 ± 0.005 | |
| imidazole | | |
| 1-(3-aminopropyl)-imidazole | 0.41 ± 0.01 | |
| 1-phenylimidazole | no inhibition | |
| 1,1′-sulfonyldiimidazole | no inhibition | |
| C-4(5) DERIVATIVES | | |
| N-omega-acetylhistamine | 0.017 ± 0.001 | |
| L-histidinamide | 0.56 ± 0.04 | |
| H-His-Trp-OH | 0.60 ± 0.03 | |
| L-histidinol | 1.53 ± 0.12 | |
| L-histidine | 4.4 ± 0.2 | |
| 4-imidazole-carboxaldehyde | 7.6 ± 0.7 | |
| imidazole-4-carbonic acid | 14.5 ± 0.6 | |
| methylester | | |
| L-histamine | 0.85 ± 0.04 | |
| C-4,5 derivatives | | |
| 5-hydroxymethyl-4-methyl- | 0.129 ± 0.005 | |
| imidazole | | |
| 4-amino-imidazole-5-carbonic | 15.5 ± 0.5 | |
| acid amide | | |
| 4,5-diphenyl-imidazole | no inhibition | |
| 4,5-dicyanoimidazole | no inhibition | |
| C-2 DERIVATIVES | | |
| 2-methyl-benzylimidazole | 0.165 ± 0.004 | |
| 2-ethyl-4-methyl-imidazole | 0.58 ± 0.04 | |
| 2-aminobenzimidazole | 1.8 ± 0.1 | |
| 2-chioro-1H-benzimidazole | no inhibition | |
| Others | | |
| 3-(1H-imidazol-1-yl)-1-(3- methylbenzo[b]thiophene-2- yl)propan-1-one | 0.0025 ± 0.0001 | 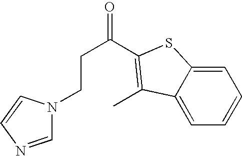 |
| 4-[(1-methyl-1H-imidazol-5- yl)methyl]-3- propyldihydrofuran-2-(3H)-one | 0.0067 ± 0.0003 | 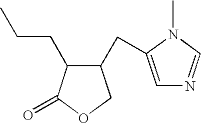 |
| 4-[2-(1H-imidazol-1-yl)- ethoxy]benzoic acid | 0.0034 ± 0.0001 | 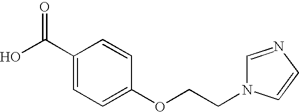 |
| 3-[3-(1H-imidazol-1-yl)propyl]- 2-thioxoimidazolidin-4-one | 0.00041 ± 0.00001 | 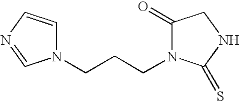 |
| 5-nitro-2-[2-([{3-(1H-imidazol- 1-yl-)propyl}amino]carbonyl)phenyl]furamide | 0.0066 ± 0.0004 | 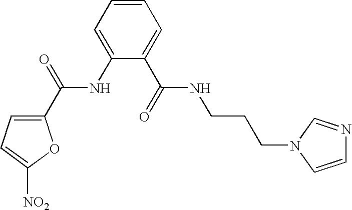 |
| N-(4-chlorophenyl)-N′-[2-(1H- imidazol-1-yl)ethyl]thiourea | 0.00165 ± 0.00007 | 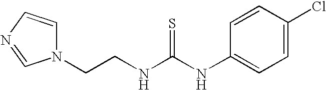 |
| 2-[(5-imidazol-1-ylmethyl- pyrrolidine-2-carbonyl)-amino]- propionic acid methyl ester | 0.0322 ± 0.0007 | 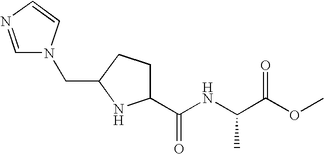 |
| 2-[(5-Imidazol-1-ylmethyl-2,3- dihydro-1H-pyrrole-2-carbonyl)- amino[-propionic acid methyl ester | n.d. | 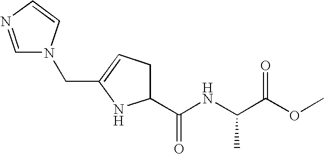 |
| Imidazo[1.5a]pyridine | 0.0356 ± 0.0005 | 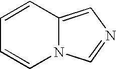 |
| Methyl (2S)-2-{[(28)-2-amino-5- (1H-imidazol-1-ylamino)-5- oxopentanoyl]amino}-3- methylbutanoate | 0.164 ± 0.004 | 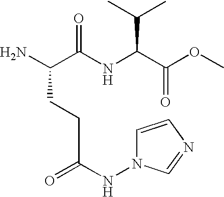 |

TABLE 4
QC inhibition by L-histamine and its two
biological metabolites (also
known as tele-methylhistamine).

| Compound | Kᵢ value (mM) | Structure |
|---|---|---|
| L-histamine | 0.85 ± 0.04 | 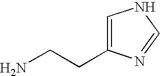 |
| 3-methyl-4-(β- aminoethyl)- imidazole | 0.120 ± 0.004 | 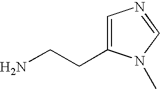 |
| 1-methyl-4-(β- aminoethyl)- imidazole | n.i | 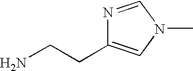 |

In a more preferred embodiment, the compound of the general formula (I) according to the present invention, optionally in combination with a glutaminyl cyclase inhibitor, is used in the form of a composition or a pharmaceutical composition according to any one of the preceding embodiments for the preparation of a medicament for the treatment of non-insulin dependent diabetes mellitus (type 2), for the improvement of impaired glucose tolerance (IGT), impaired fasting glucose (IFG) and impaired glucose metabolism (IGM) by lowering elevated blood glucose levels in response to an oral glucose challenge, for the treatment of glucosuria, and disturbances of signal action at the cells of the islets of Langerhans and insulin sensitivity in the peripheral tissue in the postprandial phase of mammals, especially in humans.

In a further preferred embodiment, the compound of the general formula (I) according to the present invention, optionally in combination with a glutaminyl cyclase inhibitor, is used in the form of a composition or a pharmaceutical composition according to any one of the preceding embodiments for the preparation of a medicament for the treatment of hyperlipidemia, metabilic acidosis, diabetic neurophaty and nephropohathy and of sequelae caused by diabetes mellitus in mammals, metabolism-related hypertension and cardiovascular sequelae caused by hypertension in mammals; for the prophylaxis or treatment of skin diseases and diseases of the mucosae, autoimmune diseases and inflammatory conditions, and for the prophylaxis or treatment of psychosomatic, neuropsychiatric and depressive illness, and neurodegenerative diseases such as anxiety, depression, sleep disorders, chronic fatigue, schizophrenia, epilepsy, nutritional disorders, spasm and chronic pain.

In a preferred embodiment, the compounds according to the invention and their corresponding pharmaceutically acceptable acid addition salt forms, are useful in treating conditions mediated by DPIV or DPIV-like enzymes, such as arthritis, obesity, immune and autoimmune disorders, allograft transplantation, cancer, neuronal disorders and dermal diseases.

Furthermore, an embodiment of the present invention comprises a simple method for the treatment of those disorders.

### EXAMPLES

The present invention can be carried out by the following examples, which are illustrating, but not limiting the scope of the invention.

Examples for Prolin Mimetics of Formula (II):

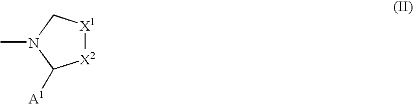

(100) Compound according to general formula (I) containing L-α-glutamine or L-α-homoglutamine, wherein X¹═CR⁵¹R⁵² and R⁵¹═H and R²═H and X²═CR⁵⁴R⁵⁵ and R⁵⁴═H and R⁵⁵═H and A¹=—C≡N, namely glutaminyl-1N-(2-cyano-pyrrolidine).

(101) Compound according to general formula (I) containing L-α-glutamine or L-α-homoglutamine, wherein X¹═S and X²═CR⁵⁴R⁵⁵ and R⁵⁴═H and R⁵⁵═H and A¹=—C≡N, namely glutaminyl-3N-(4-cyano-thiazolidine).

(102) Compound according to general formula (I) containing L-α-glutamine or L-α-homoglutamine, wherein X¹═SO and X²═CR⁵⁴R⁵⁵ and R⁵⁴═H and R⁵⁵═H and A¹=—C═N, namely glutaminyl-3N-(4-cyano-1-oxo-thiazolidine).

(103) Compound according to general formula (I) containing L-α-glutamine or L-α-homoglutamine, wherein X¹═SO₂ and X²═CR⁵⁴R⁵⁵ and R⁵⁴═H and R¹⁵═H and A¹=—C≡N, namely glutaminyl-3N-(4-cyano-1-dioxo-thiazolidine).

(104) Compound according to general formula (I) containing L-α-glutamine or L-α-homoglutamine, wherein X¹═SO and X²═CR⁵⁴R⁵⁵ and R⁵⁴═H and R⁵⁵═H and A¹=H, namely glutaminyl-3N-(1-oxo-thiazolidine).

(105) Compound according to general formula (I) containing L-α-glutamine or L-α-homoglutamine, wherein X¹═SO₂ and X²═CR⁵⁴R⁵⁵ and R⁵⁴═H and R⁵⁵═H and A¹=—H, namely glutaminyl-3N-(1-dioxo-thiazolidine).

(106) Compound according to general formula (I) containing L-α-glutamine or L-α-homoglutamine, wherein X¹═NR⁵¹ and R⁵³═H and X²═CR⁵⁴R⁵⁵ and R⁵⁴═H and R⁵⁵═H and A¹=—H, namely glutaminyl-1N-(-imidazolidine).

(107) Compound according to general formula (I) containing L-α-glutamine or L-α-homoglutamine, wherein X¹═NR⁵³ and R⁵³═H and X²═CR⁵⁴, R⁵⁵ and R⁵⁴═H and R⁵⁵═H and A¹=—C≡N, namely glutaminyl-1N-(5-cyano-imidazolidine).

(108) Compound according to general formula (I) containing L-α-glutamine or L-α-homoglutamine, wherein X¹═NR⁵¹ and R⁵³═CH₃ and X²═CR⁵⁴R⁵⁵ and R⁵⁴═H and R⁵⁵═H and A¹=—H, namely glutaminyl-1N-(3N-methyl-imidazolidine).

(109) Compound according to general formula (I) containing L-α-glutamine or L-α-homoglutamine, wherein X¹═NR⁵³ and R⁵³═CH₃ and X²═CR⁵⁴R⁵⁵ and R¹⁴═H and R⁵⁵═H and A¹=—C≡N, namely glutaminyl-1N-(3N-methyl-5-cyano-imidazolidine).

(110) Compound according to general formula (I) containing L-α-glutamine or L-α-homoglutamine, wherein X¹═NR⁵³ and R⁵³═C₆H₅ and X²═CR⁵R⁵⁵ and R⁵⁴═H and R⁵⁵═H and A¹=—H, namely glutaminyl-1N-(3N-phenyl-imidazolidine).

(111) Compound according to general formula (I) containing L-α-glutamine or L-α-homoglutamine, wherein X¹═NR⁵³ and R⁵³═C₆H₅ and X²═CR⁵⁴R⁵⁵ and R⁵⁴═H and R⁵⁵═H and A¹=—C≡N, namely glutaminyl-1N-(3N-phenyl-5-cyano-imidazolidine).

(112) Compound according to general formula (I) containing L-α-glutamine or L-α-homoglutamine, wherein X¹═O and X═CR⁵⁴R⁵⁵ and R⁵⁵═H and R⁵⁵═H and A¹=—H, namely glutaminyl-3N-(oxazolidine).

(113) Compound according to general formula (I) containing L-α-glutamine or L-α-homoglutamine, wherein X¹═O and X²═CR⁵⁴R⁵⁵ and R⁵⁴═H and R⁵⁵═H and A¹=—C≡N, namely glutaminyl-3N-(4-cyano-oxazolidine).

(114) Compound according to general formula (I) containing L-α-glutamine or L-α-homoglutamine, wherein X¹═CR⁵¹R⁵² and R⁵¹═H and R⁵²═CH₃ and X²═CR⁵⁴R⁵⁵ and R⁵⁴=H and R⁵⁵═H and A¹=—C≡N, namely glutaminyl-N-(2-cyano-4-methyl-pyrrolidine).

(115) Compound according to general formula (I) containing L-α-glutamine or L-α-homoglutamine, wherein X¹═CR⁵¹R⁵² and R⁵²═CH₃ and R⁵²═CH₃ and X²═CR⁵⁴R⁵⁵ and R⁵⁴ ═H and R⁵⁵═H and A¹=—C≡N, namely glutaminyl-N-(2-cyano-4,4-dimethyl-pyrrolidine).

(116) Compound according to general formula (I) containing L-α-glutamine or L-α-homoglutamine, wherein X¹═CR⁵¹R⁵² and R⁵¹═H and R⁵²═H and X²═CR⁵¹R⁵⁵ and R⁵⁴=CH₃ and R⁵⁵═H and A¹=—C≡N, namely glutaminyl-N-(2-cyano-3-methyl-pyrrolidine).

(117) Compound according to general formula (I) containing L-α-glutamine or L-α-homoglutamine, wherein X═CR⁵¹R⁵² and R⁵¹═H and R⁵²═H and X²═CR⁴R⁵⁵ and R⁵⁴=CH₃ and R⁵⁵═CH₃ and A¹=—C≡N, namely glutaminyl-N-(2-cyano-3,3-dimethyl-pyrrolidine).

(118) Compound according to general formula (I) containing L-α-glutamine or L-α-homoglutamine, wherein X¹═CR⁵¹R⁵²R⁵² and R⁵⁵═CH₃ and R⁵²═H and X²═CR⁵⁴R⁵⁵ and R⁵⁴=H and R⁵⁵═CH₃ and A¹=—C≡N, namely glutaminyl-N-(2-cyano-3,4-dimethyl-pyrrolidine).

(119) Compound according to general formula (I) containing L-α-glutamine or L-α-homoglutamine, wherein X¹═CR⁵¹R⁵² and R⁵¹═CH₃ and R⁵²═H and X²═CR⁵⁴R⁵⁵ and R⁵⁴═H and R⁵⁵═H and A¹=—H, namely glutaminyl-N-(3-methyl-pyrrolidine).

(120) Compound according to general formula (I) containing L-α-glutamine or L-α-homoglutamine, wherein X¹═CR⁵¹R⁵² and R⁵¹═CH₃ and R⁵²═CH₃ and X²═CR⁵⁴R⁵⁵ and R⁵⁴ ═H and R⁵⁵═H and A¹=—H, namely glutaminyl-N-(3,3-dimethyl-pyrrolidine).

(121) Compound according to general formula (I) containing L-α-glutamine or L-α-homoglutamine, wherein X¹═CR⁵¹R⁵² and R⁵¹═CH₃ and R⁵²═H and X²═CR⁵⁴R⁵⁵ and R⁵⁴=CH₃ and R⁵⁵═H and A¹=—C≡N, namely glutaminyl-N-(3,4-dimethyl-pyrrolidine).

(122) Compound according to general formula (I) containing L-α-glutamine or L-α-homoglutamine, wherein X═CRR¹² and R⁵¹═CF₃ and R⁵²═H and X²═CR⁵⁴R⁵⁵ and R⁵⁴=H and R⁵⁵═H and A¹=—C≡N, namely glutaminyl-N-(2-cyano-4-trifluormethyl-pyrrolidine).

(123) Compound according to general formula (I) containing L-α-glutamine or L-α-homoglutamine, wherein X¹═CR⁵¹R⁵² and R⁵¹═H and R⁵²═H and X²═CR⁵⁴R⁵⁵ and R⁵⁴=CF₃ and R⁵⁵═H and A¹=—C≡N, namely glutaminyl-N-(2-cyano-3-trifluormethyl-pyrrolidine).

(124) Compound according to general formula (I) containing L-α-glutamine or L-α-homoglutamine, wherein X¹═CR⁵¹R⁵² and R⁵¹═CF₃ and R⁵²═H and X²═CR¹⁴R⁵⁵ and R⁵⁴═CF₃ and R⁵⁵═H and A¹=—C≡N, namely glutaminyl-N-(2-cyano-3,4-bis(trifluormethyl)-pyrrolidine).

(125) Compound according to general formula (I) containing L-α-glutamine or L-α-homoglutamine, wherein X¹═CR⁵¹R⁵² and R⁵¹═CF₃ and R⁵²═H and X²═CR⁵⁴R⁵⁵ and R⁵⁴=H and R⁵⁵═H and A¹=H, namely glutaminyl-N-(3-trifluormethyl-pyrrolidine).

(126) Compound according to general formula (I) containing L-α-glutamine or L-α-homoglutamine, wherein X═CRR¹² and R⁵¹═CF₃ and R⁵²═H and X²═CR⁵⁴R⁵⁵ and R⁵⁴═CF₃ and R⁵⁵═H and A¹=—H, namely glutaminyl-N-(3,4-bis(trifluormethyl)-pyrrolidine).

(127) Compound according to general formula (I) containing L-α-glutamine or L-α-homoglutamine, wherein X¹═CR⁵¹R⁵² and R⁵¹═H and R⁵²═H and X²=O and A¹=—C≡N, namely glutaminyl-3N-(2-cyano-oxazolidine).

(128) Compound according to general formula (I) containing L-α-glutamine or L-α-homoglutamine, wherein X¹═CR⁵¹R⁵² and R⁵²═H and R⁵²═H and X²═S and A¹=—C≡N, namely glutaminyl-3N-(2-cyano-thiazolidine).

(129) Compound according to general formula (I) containing L-α-glutamine or L-α-homoglutamine, wherein X¹═CR⁵¹R⁵² and R⁵¹═H and R⁵²═H and X²═SO and A¹=—C≡N, namely glutaminyl-3N-(2-cyano-1-oxo-thiazolidine).

(130) Compound according to general formula (I) containing L-α-glutamine or L-α-homoglutamine, wherein X¹═CR⁵¹R⁵² and R⁵¹═H and R⁵²═H and X²═SO₂ and A¹=—C≡N, namely glutaminyl-3N-(2-cyano-1,1-dioxo-thiazolidine).

(131) Compound according to general formula (I) containing L-α-glutamine or L-α-homoglutamine, wherein X¹═CR⁵¹R⁵² and R⁵¹═H and R⁵²═H and X²═NR⁵⁶ and R⁵⁶═H and A¹=—C≡N, namely glutaminyl-1N-(2-cyano-imidazolidine).

(132) Compound according to general formula (I) containing L-α-glutamine or L-α-homoglutamine, wherein X¹═CR⁵¹R⁵² and R⁵¹═H and R⁵²═H and X²═NR⁵⁶ and R⁵⁶═CH_{3 and A}¹=—C≡N, namely glutaminyl-1N-(2-cyano-3N-methyl-imidazolidine).

(133) Compound according to general formula (I) containing L-α-glutamine or L-α-homoglutamine, wherein X¹═CR⁵¹R⁵² and R⁵¹═H and R⁵²═H and X²═NR⁵⁶ and R⁵⁶═C₆H₅ and A¹=—C≡N, namely glutaminyl-1N-(2-cyano-3N-phenyl-imidazolidine).

(134) Compound according to general formula (I) containing L-α-glutamine or L-α-homoglutamine, wherein X¹═NR⁵³ and R⁵³═H and X²═NR⁵⁶ and R⁵⁶═H and A¹=—H, namely glutaminyl-4N-(1,2,4-triazolidine).

(135) Compound according to general formula (I) containing L-α-glutamine or L-α-homoglutamine, wherein X¹═NR⁵³ and R⁵³═H and X²═NR⁵⁶ and R⁵⁶═H and A¹=—C≡N, namely glutaminyl-4N-(3-cyano-1,2,4-triazolidine).

(136) Compound according to general formula (I) containing L-α-glutamine or L-α-homoglutamine, wherein X¹═NR⁵³ and R⁵³═CH₃ and X²═NR⁵⁶ and R⁵⁶═H and A¹=—H, namely glutaminyl-4N-(1N-methyl-1,2,4-triazolidine).

(137) Compound according to general formula (I) containing L-α-glutamine or L-α-homoglutamine, wherein X¹═NR⁵³ and R⁵³═CH₃ and X═NR⁵⁶ and R⁵⁶═H and A¹=—C≡N, namely glutaminyl-4N-(1N-methyl-3-cyano-1,2,4-triazolidine).

(138) Compound according to general formula (I) containing L-α-glutamine or L-α-homoglutamine, wherein X¹═NR⁵³ and R⁵³═H and X²═NR⁵⁶ and R⁵⁶═CH₃ and Al═—H, namely glutaminyl-4N-(2N-methyl-1,2,4-triazolidine).

(139) Compound according to general formula (I) containing L-α-glutamine or L-α-homoglutamine, wherein X¹═NR⁵³ and R⁵³═H and X²═NR⁵⁶ and R⁵⁶═CH₃ and A¹=—C≡N, namely glutaminyl-4N-(2N-methyl-3-cyano-1,2,4-triazolidine).

(140) Compound according to general formula (I) containing L-α-glutamine or L-α-homoglutamine, wherein X¹═NR⁵³ and R⁵³═CH₃ and X²═NR⁵⁶ and R⁵⁶═CH₃ and A¹=—C—N, namely glutaminyl-4N-(1N,2N-dimethyl-3-cyano-1,2,4-triazolidine).

(141) Compound according to general formula (I) containing L-α-glutamine or L-α-homoglutamine, wherein X¹═CR⁵¹R⁵² and R⁵¹═H and R¹²═H and X²═CR⁵⁴R⁵⁵ and R⁵⁴═H and R⁵⁵═H and A¹=—CHO, namely glutaminyl-1N-(pyrrolidine-2-carbaldehyde).

(142) Compound according to general formula (I) containing L-α-glutamine or L-α-homoglutamine, wherein X¹=S and X²═CR⁵⁴R⁵⁵ and R⁵⁴═H and R⁵⁵═H and A¹=—CHO, namely glutaminyl-3N-(thiazolidine-4-carbaldehyde).

(143) Compound according to general formula (I) containing L-α-glutamine or L-α-homoglutamine, wherein X¹═O and X²═CR⁵⁴R⁵⁵ and R⁵⁴═H and R¹⁵═H and A¹=—CHO, namely glutaminyl-3N-(oxazolidine-4-carbaldehyde).

(144) Compound according to general formula (I) containing L-α-glutamine or L-α-homoglutamine, wherein X¹═NR¹³ and R⁵³═H and X²═CR⁴R⁵ and R⁵⁴═H and R⁵⁵═H and A¹=—CHO, namely glutaminyl-1N-(imidazolidine-5-carbaldehyde).

(145) Compound according to general formula (I) containing L-α-glutamine or L-α-homoglutamine, wherein X¹═NR⁵³ and R⁵³═CH₃ and X²═CR⁵⁴R⁵⁵ and R⁵⁴═H and R⁵⁵═H and A¹=—CHO, namely glutaminyl-1N-(3N-methyl-imidazolidine-5-carbaldehyde).

(146) Compound according to general formula (I) containing L-α-glutamine or L-α-homoglutamine, wherein X¹═CR⁵¹R⁵² and R⁵¹═H and R⁵²═H and X²═CR⁵⁴R⁵⁵ and R⁵⁴═H and R⁵⁵═H and A¹=—SO₃H, namely glutaminyl-1N-(pyrrolidine-2-sulphonic acid).

(147) Compound according to general formula (I) containing L-α-glutamine or L-α-homoglutamine, wherein X¹═S and X²═CR⁵⁴R⁵⁵ and R⁵⁴═H and R⁵⁵═H and A¹=—SO₃H, namely glutaminyl-3N-(thiazolidine-4-sulphonic acid).

(148) Compound according to general formula (I) containing L-α-glutamine or L-α-homoglutamine, wherein X¹═O and X²═CR⁵⁴R⁵⁵ and R⁵⁴═H and R⁵⁵═H and A¹=—SO₃H, namely glutaminyl-3N-(oxazolidine-4-sulphonic acid).

(149) Compound according to general formula (I) containing L-α-glutamine or L-α-homoglutamine, wherein X¹═NR⁵³ and R⁵³═H and X²═CR⁵⁴R⁵⁵ and R⁵⁴═H and R⁵⁵═Hand A¹=—SO₃H, namely glutaminyl-1N-(imidazolidine-5-sulphonic acid).

(150) Compound according to general formula (I) containing L-α-glutamine or L-α-homoglutaine, wherein X¹═CR⁵¹R⁵² and R⁵³═H and R⁵²═H and X²═CR⁵⁴R⁵⁵ and R⁵⁴═H and R⁵⁵═H and A¹=—SO₂NH₂, namely glutaminyl-1N-(pyrrolidine-2-sulphonamide).

(151) Compound according to general formula (I) containing L-α-glutamine or L-α-homoglutamine, wherein X¹=S and X²═CR⁵⁴, R⁵⁴ and R⁵⁴═H and R⁵═H and A¹=—SO₂NH₂, namely glutaminyl-3N-(thiazolidine-4-sulphonamide).

(152) Compound according to general formula (I) containing L-α-glutamine or L-α-homoglutamine, wherein X¹═O and X²═CR⁵⁴R⁵⁵ and R⁵⁴═H and R⁵⁴═H and A¹=—SO₂NH₂, namely glutaminyl-3N-(oxazolidine-4-sulphonamide).

(153) Compound according to general formula (I) containing L-α-glutamine or L-α-homoglutamine, wherein X¹═NR⁵³ and R⁵³═H and X²═CR⁵⁴R⁵⁵ and R⁵⁴═H and R¹⁵═H and A¹=—SO₂NH₂, namely glutaminyl-1N-(imidazolidine-5-sulphonamide).

(154) Compound according to general formula (I) containing L-α-glutamine or L-α-homoglutamine, wherein X¹═S and X²═CR⁵⁴R⁵⁵ and R⁵⁴═H and R⁵⁵═H and A¹=—CO—NH₂, namely glutaminyl-3N-(thiazolidine-4-carboxamide).

(155) Compound according to general formula (I) containing L-α-glutamine or L-α-homoglutamine, wherein X¹═O and X²═CR⁵⁴R⁵⁵ and R⁵⁴═H and R⁵⁵═H and A¹=—CO—NH₂, namely glutaminyl-3N-(oxazolidine-4-carboxamide).

(156) Compound according to general formula (I) containing L-α-glutamine or L-α-homoglutamine, wherein X¹═NR⁵³ and R⁵³═H and X²═CR⁵⁴R⁵⁵ and R⁵⁴═H and R⁵═H and A¹=—CO—NH₂, namely glutaminyl-1N-(imidazolidine-5-carboxamide).

(157) Compound according to general formula (I) containing L-α-glutamine or L-α-homoglutamine, wherein X¹═S and X²═CR⁵⁴R⁵⁵ and R⁵⁴═H and R⁵⁵═H and A¹=—COOH, namely glutaminyl-3N-(thiazolidine-4-carboxylic acid).

(158) Compound according to general formula (I) containing L-α-glutamine or L-α-homoglutamine, wherein X¹═O and X²═CR⁵⁴R⁵⁵ and R⁵⁴═H and R⁵⁵═H and A¹=—COOH, namely glutaminyl-3N-(oxazolidine-4-carboxylic acid).

(159) Compound according to general formula (I) containing L-(X-glutamine or L-α-homoglutamine, wherein X¹═NR⁵³ and R⁵³═H and X²═CR⁵⁴R⁵⁵ and R⁵⁴═H and R⁵⁵═H and A¹=—COOH, namely glutaminyl-1N-(imidazolidine-5-carboxylic acid).

(160) Compound according to general formula (I) containing L-α-glutamine or L-α-homoglutamine, wherein X¹═CR⁵¹R⁵² and R⁵¹═H and R⁵²═H and X²═CR⁵⁴R⁵⁵ and R⁵¹═H and R⁵⁵═H and A¹=—OP(═O)(OH)₂, namely glutaminyl-1N-(pyrrolidine-2-phosphoric acid).

(161) Compound according to general formula (I) containing L-α-glutamine or L-α-homoglutamine, wherein X¹=S and X²═CR⁵⁴R⁵⁵ and R⁵⁴═H and R⁵⁵═H and A¹═—OP(═O)(OH)₂, namely glutaminyl-3N-(thiazolidine-4-phosphoric acid).

(162) Compound according to general formula (I) containing L-α-glutamine or L-α-homoglutamine, wherein X¹═O and X²═CR⁵⁴R⁵⁵ and R⁵⁴═H and R⁵⁵═H and A¹=—OP(═O)(OH)₂, namely glutaminyl-3N-(oxazolidine-4-phosphoric acid).

(163) Compound according to general formula (I) containing L-α-glutamine or L-α-homoglutamine, wherein X¹═NR⁵³ and R⁵³═H and X²═CR⁵⁵R⁵⁵ and R⁵⁴═H and R⁵⁵═H and A¹=—OP(═O)(OH)₂, namely glutaminyl-1N-(imidazolidine-5-phosphoric acid).

(164) Compound according to general formula (I) containing L-α-glutamine or L-α-homoglutamine, wherein X¹═CR⁵¹R⁵² and R⁵¹═H and R⁵²═H and X²═CR⁵⁴R⁵⁵ and R⁵⁴═H and R⁵⁵═H and A¹=—P(═O)(OH)₂, namely glutaminyl-1N-(pyrrolidine-2-phosphonic acid).

(165) Compound according to general formula (I) containing L-α-glutamine or L-α-homoglutamine, wherein X¹═S and X²═CR⁵⁴R⁵⁵ and R⁵⁴═H and R⁵⁵═H and A═—P(═O)(OH)₂, namely glutaminyl-3N-(thiazolidine-4-phosphonic acid).

(166) Compound according to general formula (I) containing L-α-glutamine or L-α-homoglutamine, wherein X¹═O and X²═CR⁵⁴R⁵⁵ and R⁵⁴═H and R⁵⁵═H and A¹═—P(═O)(OH)₂, namely glutaminyl-3N-(oxazolidine-4-phosphonic acid).

(167) Compound according to general formula (I) containing L-α-glutamine or L-α-homoglutamine, wherein X¹═NR⁵³ and R⁵³═H and X²═CR⁵⁴R⁵⁵ and R⁵⁴═H and R⁵⁵ ═H and A¹=—P(═O)(OH)₂, namely glutaminyl-1N-(imidazolidine-5-phosphonic acid).

(168) Compound according to general formula (I) containing L-α-glutamine or L-α-homoglutamine, wherein X¹═CR⁵¹R⁵² and R^{5l} ═H and R⁵²═H and X²═CR⁵⁴R⁵⁵ and R⁵⁴═H and R⁵⁵═H and A¹=—P(═O)(OR⁷⁶)(OR⁷⁷) and R⁷⁶═—C₆H₅, and R⁷⁷═—C₆H₅, namely glutaminyl-(pyrrolidine-2-phosphonic acid diphenyl ester).

(169) Compound according to general formula (I) containing L-α-glutamine or L-α-homoglutamine, wherein X¹=S and X²═CR⁵⁴R⁵¹ and R⁵⁴═H and R⁵¹═H and A¹=—P(═O)(OR⁷⁶)(OR⁷⁷) and R⁷⁶═—C₆H₅, and R⁷⁷═—C₆H₅, namely glutaminyl-3N-(thiazolidine-4-phosphonic acid diphenyl ester).

(170) Compound according to general formula (I) containing L-α-glutamine or L-α-homoglutamine, wherein X]=0 and X²═CR⁴R⁵ and R⁵⁴═H and R⁵¹═H and A¹=—P(═O)(OR⁷⁶)(OR⁷⁷) and R⁷⁶═—C₆H₅, and R⁷⁷═—C₆H₅, namely glutaminyl-3N-(oxazolidine-4-phosphonic acid diphenyl ester).

(171) Compound according to general formula (I) containing L-α-glutamine or L-α-homoglutamine, wherein X¹═NR⁵³ and R⁵³═H and X²═CR⁵R⁵ and R⁵⁴═H and R⁵⁵═H and A¹=—P(═O)(OR⁷⁶)(OR⁷⁷) and R⁷⁶═—C₆H₅, and R⁷⁷═—C₆H₅, namely glutaminyl-1N-(imidazolidine-5-phosphonic acid diphenyl ester).

(172) Compound according to general formula (I) containing L-α-glutamine or L-α-homoglutamine, wherein X¹═CR⁵¹R⁵² and R⁵¹═H and R⁵²═H and X²═CR⁵⁴R⁵⁵ and R⁵⁴═H and R⁵⁵═H and A¹=2H-tetrazol-5-yl, namely glutaminyl-1N-(2-(2H-tetrazol-5-yl)-pyrrolidine).

(173) Compound according to general formula (I) containing L-α-glutamine or L-α-homoglutamine, wherein X¹═S and X²═CR⁵⁴R⁵⁵ and R⁵⁴═H and R⁵⁵═H and A¹=2H-tetrazol-5-yl, namely glutaminyl-3N-(4-(2H-tetrazol-5-7yl)-thiazolidine).

(174) Compound according to general formula (I) containing L-α-glutamine or L-α-homoglutamine, wherein X¹═O and X²═CR⁵⁴R⁵⁵ and R⁵⁴═H and R⁵⁵═H and A¹=2H-tetrazol-5-yl, namely glutaminyl-3N-(4-(2H-tetrazol-5-yl)-oxazolidine).

(175) Compound according to general formula (I) containing L-α-glutamine or L-α-homoglutamine, wherein X¹═NR⁵³ and R⁵³═H and X²═CR⁵⁴, R⁵⁵ and R⁵⁴═H and R⁵⁵═H and A¹=2H-tetrazol-5-yl, namely glutaminyl-1N-(5-(2H-tetrazol-5-yl)-imidazolidine).

(176) Compound according to general formula (I) containing L-α-glutamine or L-α-homoglutamine, wherein X¹═CR⁵¹R⁵² and R⁵¹═H and R⁵²═H and X²═CR⁵⁴R⁵⁵ and R⁵⁴═H and R⁵⁵═H and A¹=—B(OH)₂, namely glutaminyl-1N-(2-(boronic acid)-pyrrolidine).

(177) Compound according to general formula (I) containing L-α-glutamine or L-α-homoglutamine, wherein X¹=S and X²═CR⁵⁴R⁵⁵ and R⁵⁴ ═H and R⁵⁵ H and A¹=—B(OH)₂, namely glutaminyl-3N-(4-(boronic acid)-thiazolidine).

(178) Compound according to general formula (I) containing L-α-glutamine or L-α-homoglutamine, wherein X¹═O and X²═CR⁵⁴R⁵⁵ and R⁵⁴═H and R⁵⁵═H and A¹=—B(OH)₂, namely glutaminyl-3N-(4-(boronic acid)-oxazolidine).

(179) Compound according to general formula (I) containing L-α-glutamine or L-α-homoglutamine, wherein X¹═NR⁵³ and R⁵³ H and X²═CR⁵⁴R⁵⁴ and R⁵⁵H and A¹=—B(OH)₂, namely glutaminyl-1N-(5-(boronic acid)-imidazolidine).

(180) Compound according to general formula (I) containing L-α-glutamine or L-α-homoglutamine, wherein X¹═CR⁵¹R⁵² and R⁵¹═H and R⁵²═H and X²═S and A¹=—COOH, namely glutaminyl-3N-(thiazolidine-2-carboxylic acid).

(181) Compound according to general formula (I) containing L-α-glutamine or L-α-homoglutamine, wherein X¹═CR⁵¹R¹² and R⁵¹═H and R⁵²═H and X²═O and A¹=—COOH, namely glutaminyl-3N-(oxazolidine-2-carboxylic acid).

(182) Compound according to general formula (I) containing L-α-glutamine or L-α-homoglutamine, wherein X¹═CR⁵¹R⁵² and R⁵¹═H and R⁵²═H and X²═NR⁵⁶ and R⁵⁶═H and A¹=—COOH, namely glutaminyl-1N-(imidazolidine-2-carboxylic acid).

(183) Compound according to general formula (I) containing L-α-glutamine or L-α-homoglutamine, wherein X¹═CR⁵¹R⁵² and R⁵¹═H and R⁵²═H and X²=S and A¹=2H-tetrazol-5-yl, namely glutaminyl-3N-(2-(2H-tetrazol-5-yl)-thiazolidine).

(184) Compound according to general formula (I) containing L-α-glutamine or L-α-homoglutamine, wherein X¹═CR⁵¹R⁵² and R⁵¹═H and R⁵²═H and X²=O and A¹=2H-tetrazol-5-yl, namely glutaminyl-3N-(2-(2H-tetrazol-5-yl)-oxazolidine).

(185) Compound according to general formula (I) containing L-α-glutamine or L-α-homoglutamine, wherein X¹═CR⁵¹R⁵² and R⁵¹═H and R⁵²═H and X²═NR⁵⁵ and R⁵⁶═H and A¹=2H-tetrazol-5-yl, namely glutaminyl-1N-(2-(2H-tetrazol-5-yl)-imidazolidine).

Examples for Prolin Mimetics of Formula (III):

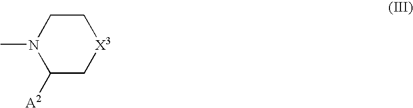

(300) Compound according to general formula (I) containing L-α-glutamine or L-α-homoglutamine, wherein X³═CR¹³¹R¹³² and R¹³¹═H and R¹³²═H and A²=—H, namely glutaminyl-1N-(piperidine).

(301) Compound according to general formula (I) containing L-α-glutamine or L-α-homoglutamine, wherein X³═O and A²=—H, namely glutaminyl-4N-(morpholine).

(302) Compound according to general formula (1) containing L-α-glutamine or L-α-homoglutamine, wherein X³═S and A²=—H, namely glutaminyl-4N-(thiomorpholine).

(303) Compound according to general formula (I) containing L-α-glutamine or L-α-homoglutamine, wherein X³═SO and A²=—H, namely glutaminyl-4N-(1-oxo-thiomorpholine).

(304) Compound according to general formula (I) containing L-α-glutamine or L-α-homoglutamine, wherein X³═SO₂ and A²=—H, namely glutaminyl-4N-(1,1-dioxo-thiomorpholine).

(305) Compound according to general formula (I) containing L-α-glutamine or L-α-homoglutamine, wherein X³═NR¹³³ and R¹³³═H and A²=—H, namely glutaminyl-1N-(piperazine).

(306) Compound according to general formula (I) containing L-α-glutamine or L-α-homoglutamine, wherein X³═NR¹³³ and R¹³³═CH₃ and A²=—H, namely glutaminyl-1N-(4-methyl-piperazine).

(307) Compound according to general formula (I) containing L-α-glutamine or L-α-homoglutamine, wherein X³═NR¹³³ and R¹³³═C₆H₅ and A²=—H, namely glutaminyl-1N-(4-phenyl-piperazine).

(308) Compound according to general formula (I) containing L-α-glutamine or L-α-homoglutamine, wherein X³═CR¹³¹R¹³² and R¹³¹═CH₃ and R¹³²═H and A²=—H, namely glutaminyl-1N-(4-methyl-piperidine).

(309) Compound according to general formula (I) containing L-α-glutamine or L-α-homoglutamine, wherein X³═CR¹³¹R¹³² and R¹³¹═CF₃ and R¹³²═H and A²=—H, namely glutaminyl-1 N-(4-trifluormethyl-piperidine).

(310) Compound according to general formula (I) containing L-α-glutamine or L-α-homoglutamine, wherein X³═CR¹³¹R¹³² and R¹³¹═C₆H⁵, and R¹³²═H and A═—H, namely glutaminyl-1N-(4-phenyl-piperidine).

(311) Compound according to general formula (I) containing L-α-glutamine or L-α-homoglutamine, wherein X³═CR¹³¹R¹³² and R¹³¹═NH₂ and R¹³²═H and A²=—H, namely glutaminyl-1N-(4-amino-piperidine).

(312) Compound according to general formula (I) containing L-α-glutamine or L-α-homoglutamine, wherein X³═CR¹³¹R¹³² and R¹³¹═H and R¹³²═H and A²=—C≡N, namely glutaminyl-1N-(2-cyano-piperidine).

(313) Compound according to general formula (I) containing L-α-glutamine or L-α-homoglutamine, wherein X³=O and A²=—C≡N, namely glutaminyl-4N-(3-cyano-morpholine).

(314) Compound according to general formula (I) containing L-α-glutamine or L-α-homoglutamine, wherein X³═S and A²=—C≡N, namely glutaminyl-4N-(3-cyano-4-thiomorpholine).

(315) Compound according to general formula (I) containing L-α-glutamine or L-α-homoglutamine, wherein X³═NR¹³³ and R¹³³═H and A²=—C═N, namely glutaminyl-1N-(2-cyano-piperazine).

(316) Compound according to general formula (I) containing L-α-glutamine or L-α-homoglutamine, wherein X³═NR¹³³ and R¹³³═CH₃ and A²=—C≡N, namely glutaminyl-1N-(2-cyano-4-methyl-piperazine).

(317) Compound according to general formula (I) containing L-α-glutamine or L-α-homoglutamine, wherein X³═NR¹³³ and R¹³³═C₆H₅ and A²=—C≡N, namely glutaminyl-1N-(2-cyano-4-phenyl-piperazine).

(318) Compound according to general formula (I) containing L-α-glutamine or L-α-homoglutamine, wherein X³═CR¹³¹R¹³² and R¹³¹═CH₃ and R¹³²═H and A²=—C≡N, namely glutaminyl-1N-(2-cyano-4-methyl-piperidine).

(319) Compound according to general formula (I) containing L-α-glutamine or L-α-homoglutamine, wherein X═CR¹³¹R¹³² and R¹³¹═CF₃ and R³²═H and A²=—C≡N, namely glutaminyl-1N-(2-cyano-4-trifluormethyl-piperidine).

(320) Compound according to general formula (I) containing L-α-glutamine or L-α-homoglutamine, wherein X³═CR¹³¹R¹³² and R¹³¹═C₆H, and R¹³²═H and A²=—C≡N, namely glutaminyl-1N-(2-cyano-4-phenyl-piperidine).

(321) Compound according to general formula (I) containing L-α-glutamine or L-α-homoglutamine, wherein X³═CR¹³¹R¹³² and R¹³¹═NH₂ and R¹³²═H and A²=—C═N, namely glutaminyl-1N-(2-cyano-4-amino-piperidine).

(322) Compound according to general formula (I) containing L-α-glutamine or L-α-homoglutamine, wherein X³═CR¹³‘R’³² and R¹³¹═H and R¹³²═H and A²=—COOH, namely glutaminyl-1 N-(piperidine-2-carboxylic acid).

(323) Compound according to general formula (I) containing L-α-glutamine or L-α-homoglutamine, wherein X³═O and A²=—COOH, namely glutaminyl-4N-(morpholine-3-carboxylic acid).

(324) Compound according to general formula (I) containing L-α-glutamine or L-α-homoglutamine, wherein X³═S and A²=—COOH, namely glutaminyl-4N-(thiomorpholine-3-carboxylic acid).

(325) Compound according to general formula (I) containing L-α-glutamine or L-α-homoglutamine, wherein X³═NR¹³³ and R¹³³═H and A²=—COOH, namely glutaminyl-1N-(piperazine-2-carboxylic acid).

(326) Compound according to general formula (I) containing L-α-glutamine or L-α-homoglutamine, wherein X³═NR¹³³ and R¹³³═CH₃ and A²=—COOH, namely glutaminyl-1N-(4-methyl-piperazine-2-carboxylic acid).

(327) Compound according to general formula (I) containing L-α-glutamine or L-α-homoglutamine, wherein X³═NR¹³³ and R¹³³═C₆H₅ and A¹=—COOH, namely glutaminyl-1N-(4-phenyl-piperazine-2-carboxylic acid).

(328) Compound according to general formula (I) containing L-α-glutamine or L-α-homoglutamine, wherein X³═CR¹³¹R¹³² and R¹³¹═CH₃ and R¹³²═H and A²=—COOH, namely glutaminyl-1 N-(4-methyl-piperidine-2-carboxylic acid).

(329) Compound according to general formula (I) containing L-α-glutamine or L-α-homoglutamine, wherein X³═CR¹¹R¹³²² ³² and R¹³¹═CF₃ and R¹³²═H and A²=—COOH, namely glutaminyl-1 N-(4-trifluormethyl-piperidine-2-carboxylic acid).

(330) Compound according to general formula (I) containing L-α-glutamine or L-α-homoglutamine, wherein X³═CR¹³¹R¹³² and R¹³¹═C₆H₅, and R¹³²═H and A²═—COOH, namely glutaminyl-1N-(4-phenyl-piperidine-2-carboxylic acid).

(331) Compound according to general formula (I) containing L-(X-glutamine or L-α-homoglutamine, wherein X³═CR¹³¹R¹³² and R¹³¹═NH₂ and R¹³²═H and A²=—COOH, namely glutarinyl-1N-(4-amino-piperidine-2-carboxylic acid).

(332) Compound according to general formula (I) containing L-α-glutamine or L-α-homoglutamine, wherein X³═CR¹¹R¹³² and R¹³¹═H and R¹³²═H and A²=—B(OH)₂, namely glutaminyl-1N-(piperidine-2-boronic acid).

(333) Compound according to general formula (I) containing L-α-glutamine or L-α-homoglutamine, wherein X³═O and A²=—B(OH)₂, namely glutaminyl-4N-(morpholine-3-boronic acid).

(334) Compound according to general formula (I) containing L-α-glutamine or L-α-homoglutamine, wherein X³═S and A²=—B(OH)₂, namely glutaminyl-4N-(thiomorpholine-3-boronic acid).

(335) Compound according to general formula (I) containing L-α-glutamine or L-α-homoglutamine, wherein X³═NR¹³³ and R¹³³═H and A²=B(OH)₂, namely glutaminyl-1N-(piperazine-2-boronic acid).

(336) Compound according to general formula (I) containing L-α-glutamine or L-α-homoglutamine, wherein X³═NR¹³³ and R¹³³═CH₃ and A²=—B(OH)₂, namely glutaminyl-1N-(4-methyl-piperazine-2-boronic acid).

(337) Compound according to general formula (I) containing L-α-glutamine or L-α-homoglutamine, wherein X³═NR¹³³ and R¹³³═C₆H₅ and A²=—B(OH)₂, namely glutaminyl-1N-(4-phenyl-piperazine-2-boronic acid).

(338) Compound according to general formula (I) containing L-α-glutamine or L-α-homoglutamine, wherein X³═CR¹³¹R¹³² and R¹³¹═CH₃ and R¹³²═H and A²=—B(OH)₂, namely glutaminyl-1N-(4-methyl-piperidine-2-boronic acid).

(339) Compound according to general formula (I) containing L-α-glutamine or L-α-homoglutamine, wherein X³═CR¹³¹R¹³² and R¹³¹═CF₃ and R¹³²═H and A²=—B(OH)₂, namely glutaminyl-1N-(4-trifluormethyl-piperidine-2-boronic acid).

(340) Compound according to general formula (I) containing L-α-glutamine or L-α-homoglutamine, wherein X³═CR¹³¹R¹³² and R¹³¹═C₆H₅ and R¹³²═H and A¹=—B(OH)₂, namely glutaminyl-1N-(4-phenyl-piperidine-2-boronic acid).

(341) Compound according to general formula (I) containing L-α-glutamine or L-α-homoglutamine, wherein X³═CR¹³¹R¹³² and R¹³¹═NH₂ and R¹³²═H and A²=—B(OH)₂, namely glutaminyl-1 N-(4-amino-piperidine-2-boronic acid).

(342) Compound according to general formula (I) containing L-α-glutamine or L-α-homoglutamine, wherein X³═CR¹³¹R¹³² and R¹³¹═H and R¹³²═H and A²=—P(═O)(OR¹⁹⁶)(OR¹⁹⁷) and R¹⁹⁶═—C₆H₅, and R¹⁹⁷═—C₆H₅, namely glutaminyl-1N-(piperidine-2-phosphonic acid diphenyl ester).

(343) Compound according to general formula (I) containing L-α-glutamine or L-α-homoglutamine, wherein X³═O and A²=—P(═O)(OR¹⁹⁶)(OR¹⁹⁷) and R¹⁹⁶═—C₆H₅, and R¹⁹⁷=—C₆H₅, namely glutaminyl-4N-(morpholine-3-phosphonic acid diphenyl ester).

(344) Compound according to general formula (I) containing L-α-glutamine or L-α-homoglutamine, wherein X³═S and A²=—P(═O)(OR¹⁹⁶)(OR¹⁹⁷) and R¹⁹⁶═—C₆H₅, and R¹⁹⁷=—C₆H₅, namely glutaminyl-4N-(thiomorpholine-3-phosphonic acid diphenyl ester).

(345) Compound according to general formula (I) containing L-α-glutamine or L-α-homoglutamine, wherein X³═NR¹³³ and R¹³³═H and A²=—P(═O)(OR¹⁹⁶)(OR¹⁹⁷) and R¹⁹⁶═—C₆H₅, and R¹⁹⁷═—C₆H₅, namely glutaminyl-1N-(piperazine-2-phosphonic acid diphenyl ester).

(346) Compound according to general formula (I) containing L-α-glutamine or L-α-homoglutamine, wherein X³═NR¹³³ and R¹³³═CH₃ and A²=—P(═O)(OR¹⁹⁶)(OR¹⁹⁷) and R⁹⁶═—C₆H₅, and R¹⁹⁷═—C₆H₅, namely glutaminyl-1N-(4-methyl-piperazine-2-phosphonic acid diphenyl ester).

(347) Compound according to general formula (I) containing L-α-glutamine or L-α-homoglutamine, wherein X³═CR³RR¹² and R¹³¹═NH₂ and R¹³²═H and A²═—P(═O)(OR¹⁹⁶)(OR¹⁹⁷) and R¹⁹⁶═—C₆H₅, and R¹⁹⁷═—C₆H₅, namely glutaminyl-1N-(4-amino-piperidine-2-phosphonic acid diphenyl ester).

(348) Compound according to general formula (I) containing L-α-glutamine or L-α-homoglutamine, wherein X³═CR¹³¹R¹³² and R¹³¹═H and R¹³²═H and A²=2H-tetrazol-5-yl, namely glutaminyl-1N-(2-(2H-tetrazol-5-yl)-piperidine)

(349) Compound according to general formula (I) containing L-α-glutamine or L-α--homoglutamine, wherein X³═O and A²=2H-tetrazol-5-yl, namely glutaminyl-4N-(3-(2H-tetrazol-5-yl)-morpholine).

(350) Compound according to general formula (I) containing L-α-glutamine or L-α-homoglutamine, wherein X³═S and A²=2H-tetrazol-5-yl, namely glutaminyl-4N-(3-(2H-tetrazol-5-yl)-thiomorpholine).

(351) Compound according to general formula (I) containing L-α-glutamine or L-α-homoglutamine, wherein X³═NR¹³³ and R¹³³═H and A²=2H-tetrazol-5-yl, namely glutaminyl-1N-(2-(2H-tetrazol-5-yl)-piperazine).

(352) Compound according to general formula (I) containing L-α-glutamine or L-α-homoglutamine, wherein X³═NR¹³³ and R³³═CH₃ and A²=2H-tetrazol-5-yl, namely glutaminyl-1N-(2-(2H-tetrazol-5-yl)-4-methyl-piperazine).

(353) Compound according to general formula (I) containing L-α-glutamine or L-α-homoglutamine, wherein X³═CR¹³¹R¹³² and R¹³¹═NH₂ and R¹³²═H and A²=2H-tetrazol-5-yl, namely glutaminyl-1N-(2-(2H-tetrazol-5-yl)-4-amino-piperidine).

Examples for Prolin Mimetics of Formula (IV):

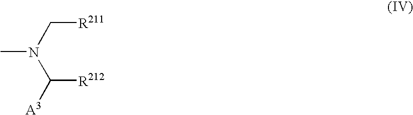

(400) Compound according to general formula (I) containing L-α-glutamine or L-α-homoglutamine, wherein R²¹¹═H and R²¹²═H and A³=—H, namely glutaminyl-(N,N-dimethylamid).

(401) Compound according to general formula (I) containing L-α-glutamine or L-α-homoglutamine, wherein R²¹¹═—CH₃ and R²¹²═H and A³=—H, namely glutaminyl-(N-ethyl-N-methylamid).

(402) Compound according to general formula (I) containing L-α-glutamine or L-α-homoglutamine, wherein R²¹¹═—C₂H₅ and R²¹²═H and A³=—H, namely glutaminyl-(N-propyl-N-methylamid).

(403) Compound according to general formula (I) containing L-α-glutamine or L-αhomoglutamine, wherein R²¹¹═—C₆H₅ and R²¹²═H and A³═—H, namely glutaminyl-(N-benzyl-N-methylamid).

(404) Compound according to general formula (I) containing L-α-glutamine or L-α-homoglutamine, wherein R²¹¹═—CH₂C₆H₅ and R²¹²═H and A³=—H, namely glutaminyl-(N-15 phenethyl-N-methylamid).

(405) Compound according to general formula (I) containing L-α-glutamine or L-αhomoglutamine, wherein R²¹¹═—CH₃ and R²¹²═CH₃ and A³═—H, namely glutaminyl-(N,N-diethylamid).

(406) Compound according to general formula (I) containing L-α-glutamine or L-α-homoglutamine, wherein R²¹¹═—C₂H₅ and R²¹²═CH₃ and A³=—H, namely glutaminyl-(N-propyl-N-ethylamid).

(407) Compound according to general formula (I) containing L-α-glutamine or L-α-homoglutamine, wherein R²¹¹═—C₆H₅ and R²¹²═CH₃ and A³=—H, namely glutaminyl-(N-benzyl-N-ethylamid).

(408) Compound according to general formula (I) containing L-α-glutamine or L-αhomoglutamine, wherein R²¹¹═—CH₂C₆H₅ and R²¹²═CH₃ and A³═—H, namely glutaminyl-(N-phenethyl-N-ethylamid).

(409) Compound according to general formula (I) containing L-α-glutamine or L-α-homoglutamine wherein R²¹¹═—C₂H₅ and R²¹²═—C₂H₅ and A³=—H, namely glutaminyl-(N,N-dipropylamid).

(410) Compound according to general formula (I) containing L-α-glutamine or L-α-homoglutamine wherein R²¹¹═—C₆H₅ and R²¹²═—C₂H₅ and A³=—H, namely glutaminyl-(N-benzy-N-propylamid).

(411) Compound according to general formula (I) containing L-α-glutamine or L-α-homoglutamine wherein R²¹¹═-CH₂C₆H₅ and R²¹²═—C₂H, and A³═—H, namely glutaminyl-(N-phenethyl-N-propylamid).

(412) Compound according to general formula (I) containing L-(X-glutamine or L-α-homoglutamine wherein R²¹¹═-C₆H₅ and R²¹²═—C₆H₅ and A═—H, namely glutaminyl-(N,N-dibenzylamid).

(413) Compound according to general formula (I) containing L-α-glutamine or L-α-homoglutamine wherein R²¹¹═-CH₂C₆H₅ and R²¹²═—C₆H₅ and A³=—H, namely glutaminyl-(N-phenyethyl-N-benylamid).

(414) Compound according to general formula (I) containing L-α-glutamine or L-α-homoglutamine wherein R²¹¹═-CH₂C₆H₅ and R²¹²═—CH₂C₆H₅ and A═—H, namely glutaminyl-(N,N-di(ohenethyl)amid).

(415) Compound according to general formula (I) containing L-α-glutamine or L-α-homoglutamine wherein R²¹¹═-H and R²¹²═H and A³=2H-tetrazol-5-yl, namely glutaminyl-(N-methyl-N-((2H-tetarzol-5-yl)methyl)amid).

(416) Compound according to general formula (I) containing L-α-glutamine or L-α-homoglutamine wherein R²¹¹═-CH₃ and R²¹²═H and A³=2H-tetrazol-5-yl, namely glutaminyl-(N-ethyl-N-((2H-tetrazol-5-yl)methyl)amid).

(417) Compound according to general formula (I) containing L-α-glutamine or L-α-homoglutamine wherein R²¹¹═-C₂H₅ and R²¹²═H and A³=2H-tetrazol-5-yl, namely glutaminyl-(N-propyl-N-((2H-tetrazol-5-yl)methyl)amid).

(418) Compound according to general formula (I) containing L-α-glutamine or L-α-homoglutamine wherein R²¹¹═-C₆H₅ and R²¹²═H and A³=2H-tetrazol-5-yl, namely glutaminyl-(N-benzyl-N-((2H-tetrazol-5-yl)methyl)amid).

(419) Compound according to general formula (I) containing L-α-glutamine or L-α-homoglutamine wherein R²¹¹═-CH₂C₆H₅ and R²¹²═H and A³=2H-tetrazol-5-yl, namely glutaminyl-(N-benzyl-N-((2H-tetrazol-5-yl)methyl)amid).

(420) Compound according to general formula (I) containing L-α-glutamine or L-α-homoglutamine wherein R²¹¹═H and R²¹²═—CH₃ and A³=2H-tetrazol-5-yl, namely glutaminyl-(N-methyl-N-(1-(2H-tetrazol-5-yl)eth-1-yl)amid).

(421) Compound according to general formula (I) containing L-α-glutamine or L-α-homoglutamine wherein R²¹¹═-H and R²¹²═—C₂H₅ and A¹=2H-tetrazol-5-yl, namely glutaminyl-(N-methyl-N-(1-(2H-tetrazol-5-yl)propyl)amid).

(422) Compound according to general formula (I) containing L-α-glutamine or L-α-homoglutamine wherein R²¹¹═H and R²¹²═—C₆H, and A³=2H-tetrazol-5-yl, namely glutaminyl-(N-methyl-N-(α-(2H-tetrazol-5-yl)benzyl)amid).

(422) Compound according to general formula (I) containing L-α-glutamine or L-α-homoglutamine wherein R²¹¹═H and R²¹²═—CH₂C₆H₅ and A³=2H-tetrazol-5-yl, namely glutaminyl-(N-methyl-N-(1-(2H-tetrazol-5-yl)-2-phenyl-eth 1-yl)amid).

(423) Compound according to general formula (I) containing L-α-glutamine or L-α-homoglutamine wherein R²¹¹═-CH₃ and R²¹²═CH₃ and A³=2H-tetrazol-5-yl, namely glutaminyl-(N-methyl-N-(1-(2H-tetrazol-5-yl)eth-1-yl)amid).

(424) Compound according to general formula (I) containing L-α-glutamine or L-α-homoglutamine wherein R²¹¹═-C₂H₅ and R²¹²═CH₃ and A³=2H-tetrazol-5-yl, namely glutaminyl-(N-methyl-N-(1-(2H-tetrazol-5-yl)eth-1-yl)amid).

(425) Compound according to general formula (I) containing L-α-glutamine or L-α-homoglutamine wherein R²¹¹═-C₆H₅ and R²¹²═CH₃ and A³=2H-tetrazol-5-yl, namely glutaminyl-(N-methyl-N-(1-(2H-tetrazol-5-yl)eth-1-yl)amid).

(426) Compound according to general formula (I) containing L-α-glutamine or L-α-homoglutamine, wherein R²¹¹═—CH₂C₆H₅ and R²¹²═CH₃ and A³=2H-tetrazol-5-yl, namely glutaminyl-(N-phenethyl-N-(1-(2H-tetrazol-5-yl)eth-1-yl)amid).

(427) Compound according to general formula (I) containing L-α-glutamine or L-α-homoglutamine, wherein R²¹¹═—CH₃ and R²¹²═—C₂H₅ and A³=2H-tetrazol-5-yl, namely glutaminyl-(N-ethyl-N-(1-(2H-tetrazol-5-yl)prop-1-yl)amid).

(428) Compound according to general formula (I) containing L-α-glutamine or L-α-homoglutamine, wherein R²¹¹═—CH₃ and R²¹²═—C₆H₅ and A³=2H-tetrazol-5-yl, namely glutaminyl-(N-ethyl-N-(ac-(2H-tetrazol-5-yl)benzyl)amid).

(429) Compound according to general formula (I) containing L-α-glutamine or L-α-homoglutamine, wherein R²¹¹═—CH₃ and R²¹²═—CH₂C₆H₅ and A³=2H-tetrazol-5-yl, namely glutaminyl-(N-ethyl-N-(1-(2H-tetrazol-5-yl)-2-phenyl-eth-1-yl)amid).

(430) Compound according to general formula (I) containing L-α-glutamine or L-α-homoglutamine, wherein R²¹¹═—C₂H₅ and R²¹²═—C₂H₅ and A³=2H-tetrazol-5-yl, namely glutaminyl-(N-propyl-N-(1-(2H-tetrazol-5-yl)prop-1-yl)amid).

(431) Compound according to general formula (I) containing L-α-glutamine or L-αhomoglutamine, wherein R²¹¹═—C₆H₅ and R²¹²═—C₂H₅ and A³=2H-tetrazol-5-yl, namely glutaminyl-(N-benzyl-N-(1-(2H-tetrazol-5-yl)prop-1-yl)amid).

(432) Compound according to general formula (I) containing L-α-glutamine or L-α-homoglutamine, wherein R²¹¹═—CH₂C₆H₅ and R²¹²═—C₂H₅ and A³=2H-tetrazol-5-yl, namely glutaminyl-(N-phenethyl-N-(1-(2H-tetrazol-5-yl)prop-1-yl)amid).

(433) Compound according to general formula (I) containing L-α-glutamine or L-α-homoglutamine, wherein R²¹¹═—C₂H₅ and R²¹²═—C₆H₅ and A³=2H-tetrazol-5-yl, namely glutaminyl-(N-propyl-N-(α-(2H-tetrazol-5-yl)benzyl)amid).

(434) Compound according to general formula (I) containing L-α-glutamine or L-α-homoglutamine, wherein R²¹¹═—C₂H₅ and R²¹²═—CH₂C₆H₅ and A³=2H-tetrazol-5-yl, namely glutaminyl-(N-propyl-N-(1-(2H-tetrazol-5-yl)₂-phenyl-eth-1-yl)amid).

(435) Compound according to general formula (I) containing L-α-glutamine or L-α-homoglutamine, wherein R²¹¹═——C₆H₅, and R²¹²═—C₆H₅ and A³=2H-tetrazol-5-yl, namely glutaminyl-(N-propyl-N-(α-(2H-tetrazol-5-yl)benzyl)amid).

(436) Compound according to general formula (I) containing L-α-glutamine or L-α-homoglutamine, wherein R²¹¹═—-CH₂C₆H₅ and R²¹²═—C₆H₅ and A³=2H-tetrazol-5-yl, namely glutaminyl-(N-propyl-N-(α(2H-tetrazol-5-yl)benzyl)amid).

(437) Compound according to general formula (I) containing L-α-glutamine or L-α-homoglutamine, wherein R²¹¹═—-C₆H₅ and R²¹²═—CH₂C₆H₅ and A³=2H-tetrazol-5-yl, namely glutaminyl-(N-propyl-N-(1-(2H-tetrazol-5-yl)-2-phenyl-eth-1-yl)amid).

(438) Compound according to general formula (I) containing L-α-glutamine or L-α-homoglutamine, wherein R²¹¹═—-CH₂C₆H₅ and R²¹²═—CH₂C₆H₅ and A³=2H-tetrazol-5-yl, namely glutaminyl-(N-propyl-N-(1-N-(1-(2H-tetrazol-5-yl)-2-phenyl-eth-1-yl)amid).

(439) Compound according to general formula (I) containing L-α-glutamine or L-α-homoglutamine, wherein R²¹¹═—H and R²¹²═H and A³═—C═N, namely glutaminyl-(N-methyl-N-(cyanomethyl)amid).

(440) Compound according to general formula (I) containing L-α-glutamine or L-α-homoglutamine, wherein R²¹¹═—-CH₃ and R²¹²═H and A³=—C═N, namely glutaminyl-(N-ethyl-N-(cyanomethyl)amid).

(441) Compound according to general formula (I) containing L-α-glutamine or L-α-homoglutamine, wherein R²¹¹═—-C₂H₅ and R²¹²═H and A³═—C═N, namely glutaminyl-(N-propyl-N-(cyanomethyl)amid).

(442) Compound according to general formula (I) containing L-α-glutamine or L-α-homoglutamine, wherein R²¹¹═—-C₆H₅ and R²¹²═H and A³=—C═N, namely glutaminyl-(N-benzyl-N-(cyanomethyl)amid).

(443) Compound according to general formula (I) containing L-α-glutamine or L-α-homoglutamine, wherein R²¹¹═—-CH₂C₆H₅ and R²¹²═H and A³═—C═N, namely glutaminyl-(N-phenethyl-N-(cyanomethyl)amid).

(444) Compound according to general formula (I) containing L-α-glutamine or L-α-homoglutamine, wherein R²¹¹═—-H and R²¹²═—CH₃ and A³=—C≡N, namely glutaminyl-(N-methyl-N-(1-cyano-eth-1-yl)amid).

(445) Compound according to general formula (I) containing L-α-glutamine or L-α-homoglutamine, wherein R²¹¹═—-H and R²¹²═—C₂H₅ and A³=—C≡N, namely glutaminyl-(N-methyl-N-(1-cyano-propyl)amid).

(446) Compound according to general formula (I) containing L-α-glutamine or L-α-homoglutamine, wherein R²¹¹═—-H and R²¹²═—C₆H₅ and A³=—C≡N, namely glutaminyl-(N-methyl-N-(α-cyano-benzyl)amid).

(447) Compound according to general formula (I) containing L-α-glutamine or L-α-homoglutamine, wherein R²¹¹═—H and R²¹²═—CH₂C₆H₅ and A³═—C≡N, namely glutaminyl-(N-methyl-N-(1-cyano-2-phenyl-eth-1-yl)amid).

(448) Compound according to general formula (I) containing L-α-glutamine or L-α-homoglutamine, wherein R²¹¹═—-CH₃ and R²¹²═CH₃ and A³═—C≡N, namely glutaminyl-(N-ethyl-N-(1-cyano-eth-1-yl)amid).

(449) Compound according to general formula (I) containing L-α-glutamine or L-α-homoglutamine, wherein R²¹¹═—-C₂H₅ and R²¹²═CH₃ and A³═—C≡N, namely glutaminyl-(N-propyl-N-(1-cyano-eth-1-yl)amid).

(450) Compound according to general formula (I) containing L-α-glutamine or L-α-homoglutamine, wherein R²¹¹═—-C₆H₅ and R²¹²═CH₃ and A³═—C≡N, namely glutaminyl-(N-benzyl-N-(1-cyano-eth-1-yl)amid).

(451) Compound according to general formula (I) containing L-α-glutamine or L-α-homoglutamine, wherein R²¹¹═—-CH₂C₆H₅ and R²¹²═CH₃ and A³=—C≡N, namely glutaminyl-(N-phenethyl-N-(1-cyano-eth-1-yl)amid).

(452) Compound according to general formula (I) containing L-α-glutamine or L-α-homoglutamine, wherein R²¹¹═—-CH₃ and R²¹²═—C₂H₅ and A³═—C≡N, namely glutaminyl-(N-ethyl-N-(1-cyano-eth-1-yl)amid).

(453) Compound according to general formula (I) containing L-α-glutamine or L-α-homoglutamine, wherein R²¹¹═—CH₃ and R²¹²═—C₆H₅ and A³=—C≡N, namely glutaminyl-(N-ethyl-N-(α-cyano-benzyl)amid).

(454) Compound according to general formula (I) containing L-α-glutamine or L-α-homoglutamine, wherein R²¹¹═—CH₃ and R²¹²═—CH₂C₆H₅ and A³=—C≡N, namely glutaminyl-(N-ethyl-N-(1-cyano-2-phenyl-eth-1-yl)amid).

(455) Compound according to general formula (I) containing L-α-glutamine or L-αhomoglutamine, wherein R²¹¹═—C₂H₅ and R²¹²═—C₂H₅ and A³═—C≡N, namely glutaminyl-(N-propyl-N-(1-cyano-prop-1-yl)amid).

(456) Compound according to general formula (I) containing L-α-glutamine or L-αhomoglutamine, wherein R²¹¹═—C₆H₅ and R²¹²═—C₂H₅ and A³═—C≡N, namely glutaminyl-(N-benzyl-N-(1-cyano-prop-1-yl)amid).

(457) Compound according to general formula (I) containing L-α-glutamine or L-αhomoglutamine, wherein R²¹¹═—CH₂C₆H₅ and R²¹²═—C₂H₅ and A³═—C≡N, namely glutaminyl-(N-phenethyl-N-(1-cyano-prop-1-yl)amid).

(458) Compound according to general formula (I) containing L-α-glutamine or L-αhomoglutamine, wherein R²¹¹═—C₂H₅ and R²¹²═—C₆H₅ and A³═—C≡N, namely glutaminyl-(N-propyl-N-(α-cyano-benzyl)amid).

(459) Compound according to general formula (I) containing L-α-glutamine or L-α-homoglutamine, wherein R²¹¹═—C₂H₅ and R²¹²═—CH₂C₆H₅ and A³═—C≡N, namely glutaminyl-(N-propyl-N-(1-cyano-2-phenyl-eth-1-yl)amid).

(460) Compound according to general formula (I) containing L-α-glutamine or L-α-homoglutamine, wherein R²¹¹═—C₆H₅ and R²¹²═—C₆H₅ and A³═—C≡N, namely glutaminyl-(N-benzyl-N-(α-cyano-benzyl)amid).

(461) Compound according to general formula (I) containing L-α-glutamine or L-αhomoglutamine, wherein R²¹¹═—CH₂C₆H₅ and R²¹²═—C₆H₅ and A³=—C≡N, namely glutaminyl-(N-phenethyl-N-(α-cyano-benzyl)amid).

(462) Compound according to general formula (I) containing L-α-glutamine or L-α-homoglutamine, wherein R²¹¹═—C₆H₅ and R²¹²═—CH₂C₆H₅ and A³=—C≡N, namely glutaminyl-(N-benzyl-N-(1-cyano-2-phenyl-eth-1-yl)amid).

(463) Compound according to general formula (I) containing L-α-glutamine or L-α-homoglutamine, wherein R²¹¹═—CH₂C₆H₅ and R²¹²═—CH₂C₆H₅ and A³=—C≡N, namely glutaminyl-(N-phenethyl-N-(1-cyano-2-phenyl-eth-1-yl)amid).

(464) Compound according to general formula (I) containing L-α-glutamine or L-α-homoglutamine, wherein R²¹¹═CF₃ and R²¹²═H and A³=—H, namely glutaminyl-(N-(2,2,2-trifluorethyl)-N-methylamid).

(465) Compound according to general formula (I) containing L-α-glutamine or L-α-homoglutamine, wherein R²¹¹═CF₃ and R²¹²═CF₃ and A³=—H, namely glutaminyl-(N,N-bis(2,2,2-trifluorethyl)amid).

(466) Compound according to general formula (I) containing L-α-glutamine or L-α-homoglutamine, wherein R²¹¹═—CH═CH₂ and R²¹²═H and A³═—H, namely glutaminyl-(N-allyl-N-methylamid).

(467) Compound according to general formula (I) containing L-α-glutamine or L-α-homoglutamine, wherein R²¹¹═—CH═CH₂ and R²¹²═CF₃ and A³=—H, namely glutaminyl-(N-allyl-N-(2,2,2-trifluorethyl)-amid).

(468) Compound according to general formula (I) containing L-α-glutamine or L-α-homoglutamine, wherein R²¹¹═H and R²¹²═—CH₃ and A³=-tetrazol-5-yl, namely glutaminyl-(N-(1-(tetrazol-5-yl)-eth-1-yl-N-methylamid).

(469) Compound according to general formula (I) containing L-α-glutamine or L-α-homoglutamine, wherein R²¹¹═—C₂H₅ and R²¹²═H and A³=-tetrazol-5-yl, namely glutaminyl-(N-(1-(tetrazol-5-yl)-methyl-N-propylamid).

(470) Compound according to general formula (I) containing L-α-glutamine or L-αhomoglutamine, wherein R²¹¹═—C₂H₅ and R²¹²═H and A³=—COOH, namely glutaminyl-(N-(carboxymethyl)-N-propylamid).

(471) Compound according to general formula (I) containing L-α-glutamine or L-α-homoglutamine, wherein R²¹═—C₆H₅ and R²¹²═H and A³=—COOH, namely glutaminyl-(N-(carboxymethyl)-N-benzylamid).

(472) Compound according to general formula (I) containing L-α-glutamine or L-α-homoglutamine, wherein R²¹¹═—C₆H₅ and R²¹²═—CH₂C₆H₅ and A³=—COOH, namely glutaminyl-(N-(1-carboxy-2-phenyl-eth-1-yl)-N-benzylamid).

(473) Compound according to general formula (I) containing L-α-glutamine or L-α-homoglutamine, wherein R²¹¹═—C₆H₅ and R²¹²═—H and A¹=—P(═O)(OR²⁹)(OR³⁰) and R²⁹=—C₆H₅ and R³⁰═—C₆H₅namely glutaminyl-(N-(methyl(O,O-diphenyl phosphonic acid ester))-N-benzylamid).

(474) Compound according to general formula (I) containing L-α-glutamine or L-α-homoglutamine, wherein R²¹¹═—H and R²¹²═—H and A³=—P(═O)(OR²⁹)(OR³⁰) and R²⁹=—C₆H₅ and R³⁰═—C₆H₅namely glutaminyl-(N-(methyl(O,O-diphenyl phosphonic acid ester))-N-methylamid).

(475) Compound according to general formula (I) containing L-α-glutamine or L-α-homoglutamine, wherein R²¹¹═—H and R²¹²═—H and A³=—C≡N namely glutaminyl-(N-(cyanomethyl)-N-methylamid).

(476) Compound according to general formula (I) containing L-α-glutamine or L-αhomoglutamine, wherein R²¹¹═—CH₃ and R²¹²═—H and A³═—C≡N namely glutaminyl-(N-(cyanomethyl)-N-ethylamid).

(477) Compound according to general formula (I) containing L-α-glutamine or L-α-homoglutamine, wherein R²¹¹═—CF₃ and R²¹²═—H and A³=—C≡N namely glutaminyl-(N-(cyanomethyl)-N-(2,2,2-trifluoroethyl)amid).

(478) Compound according to general formula (I) containing L-α-glutamine or L-α-homoglutamine, wherein R²¹¹═—C₆H₅ and R²¹²═—H and A³=—C≡N namely glutaminyl-(N-(cyanomethyl)-N-benzylamid).

(479) Compound according to general formula (I) containing L-α-glutamine or L-α-homoglutamine, wherein R²¹¹═—C₆F₅ and R²¹²═—H and A³=—C≡N namely glutaminyl-(N-(cyanomethyl)-N-(pentafluorophenylmethyl)amid).

(480) Compound according to general formula (I) containing L-α-glutamine or L-α-homoglutamine, wherein R²¹¹═—H and R²¹²═—CH₃ and A³=—C≡N namely glutaminyl-(N-(1(cyano-eth-1-yl)-N-methylamid).

(481) Compound according to general formula (I) containing L-α-glutamine or L-α-homoglutamine, wherein R²¹¹═—CH₃ and R²¹²═—CH₃ and A³═—C≡N namely glutaminyl-(N-(1(cyano-eth1-yl)-N-ethylamid).

(482) Compound according to general formula (I) containing L-α-glutamine or L-α-homoglutamine, wherein R²¹¹═—C₆H₅ and R²¹²═—CH₃ and A³═—C≡N namely glutaminyl-(N-(1-cyano-eth-1-yl)-N-benzylamid).

(483) Compound according to general formula (I) containing L-α-glutamine or L-α-homoglutamine, wherein R²¹¹═—H and R²¹²═—C₆H₅, and A³=—C≡N namely glutaminyl-(N-(α-(cyano-benzyl)-N-methylamid).

(484) Compound according to general formula (I) containing L-α-glutamine or L-α-homoglutamine, wherein R²¹¹═—H and R²¹²═—CF₃ and A³═—C≡N namely glutaminyl-(N-(1-cyano-2,2,2-trifluoreth-1-yl)-N-methylamid).

(485) Compound according to general formula (I) containing L-α-glutamine or L-α-homoglutamine, wherein R²¹¹═—H and R²¹²═—H and A³=—B(OH)₂, namely glutaminyl-(N-(methyl boronic acid)-N-methylamid).

(486) Compound according to general formula (I) containing L-α-glutamine or L-α-homoglutamine, wherein R²¹¹═—CH₃ and R²¹²═—H and A³=—B(OH)₂, namely glutaminyl-(N-(methyl boronic acid)-N-ethylamid).

(487) Compound according to general formula (I) containing L-α-glutamine or L-α-homoglutamine, wherein R²¹¹═—CF₃ and R²¹²═—H and A³=—B(OH)₂, namely glutaminyl-(N-(methyl boronic acid)-N-(2,2,2-trifluoroethyl)-amid).

(488) Compound according to general formula (I) containing L-α-glutamine or L-α-homoglutamine, wherein R²¹¹═—C₆H₅ and R²¹²═—H and A³=—B(OH)₂, namely glutaminyl-(N-(methyl boronic acid)-N-benzylamid).

(489) Compound according to general formula (I) containing L-α-glutamine or L-α-homoglutamine, wherein R²¹═—CrF₅ and R²¹²═—H and A³=—B(OH)₂, namely glutaminyl-(N-(methyl boronic acid)-N-(pentafluorophenylmethyl)amid).

(490) Compound according to general formula (I) containing L-α-glutamine or L-α-homoglutamine, wherein R²¹¹═—H and R²¹²═—CH₃ and A³=—B(OH)₂, namely glutaminyl-(N-(1-boronic acid-eth-1-yl)-N-methylamid).

(491) Compound according to general formula (I) containing L-α-glutamine or L-αhomoglutamine, wherein R²¹¹═—H and R²¹²═—C₄H₅ and A³=—B(OH)₂, namely glutaminyl-(N-(α-boronic acid)-benzyl)-N-methylamid).

Examples for Prolin Mimetics of Formula (V):

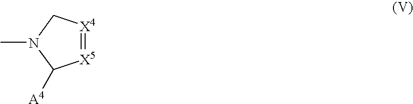

(500) Compound according to general formula (I) containing L-α-glutamine or L-α-homoglutamine, wherein X⁴═CR²⁹¹ and R²⁹¹═—H and X⁵═CR²⁹² and R²⁹²═—H and A⁴=—H, namely glutaminyl-(2,5-dihydro-1H-pyrrole).

(501) Compound according to general formula (I) containing L-α-glutamine or L-α-homoglutamine, wherein X⁴═CR²⁹¹ and R²⁹¹═—H and X⁵═CR²⁹² and R²⁹²═—H and A⁴=—COOH, namely glutaminyl-(2,5-dihydro-1H-pyrrole-2-carboxylic acid).

(502) Compound according to general formula (I) containing L-α-glutamine or L-α-homoglutamine, wherein X⁴═CR²⁹¹ and R²⁹¹═—H and X¹═CR²⁹² and R²⁹²═—H and A⁴=—CONH₂, namely glutaminyl-(2,5-dihydro-1H-pyrrole-2-carboxamide).

(503) Compound according to general formula (I) containing L-α-glutamine or L-αhomoglutamine, wherein X⁴═CR²⁹¹ and R²⁹¹═—H and X¹═CR²⁹² and R²⁹²═—H and A⁴=—B(OH)₂, namely glutaminyl-(2,5-dihydro-1H-pyrrole-2-boronic acid).

(504) Compound according to general formula (I) containing L-α-glutamine or L-αhomoglutamine, wherein X⁴═CR²⁹¹ and R²⁹¹═—H and X¹═CR²⁹² and R²⁹²═—H and A⁴=—SO₃H, namely glutaminyl-(2,5-dihydro-1H-pyrrole-2-sulphonic acid).

(505) Compound according to general formula (I) containing L-α-glutamine or L-α-homoglutamine, wherein X⁴═CR²⁹¹ and R²⁹¹═—H and X¹═CR²⁹² and R²⁹²═—H and A 4═—CF₃, namely glutaminyl-(2,5-dihydro-2-trifluoromethyl-1H-pyrrole).

(506) Compound according to general formula (I) containing L-α-glutamine or L-α-homoglutamine, wherein X⁴═CR²⁹¹ and R²⁹¹═—H and X⁵═CR²⁹² and R²⁹²═—H and A⁴=—OP(═O)(OH)₂, namely glutaminyl-(2,5-dihydro-1H-pyrrole-2-phosphoric acid).

(507) Compound according to general formula (I) containing L-α-glutamine or L-αhomoglutamine, wherein X⁴═CR²⁹¹ and R²⁹¹═—H and X⁵═CR^{292 and R}²⁹²═—H and A⁴=—P(═O)(OH)₂, namely glutaminyl-(2,5-dihydro-1H-pyrrole-2-phosphonic acid).

(508) Compound according to general formula (I) containing L-α-glutamine or L-α-homoglutamine, wherein X⁴═CR²⁹¹ and R²⁹¹═—H and X⁵═CR²⁹² and R²⁹²═—H and A⁴=—OP(═O)(OR³¹⁴)(OR³¹⁵) and R³¹⁴═—C₆H₅ and R³¹⁵═—C₆H₅, namely glutaminyl-(2,5-dihydro-1H-pyrrole-2-phosphoric acid diphenyl ester).

(509) Compound according to general formula (I) containing L-α-glutamine or L-α-homoglutamine, wherein X⁴═CR²⁹¹ and R²⁹¹═—H and X¹═CR²⁹² and R²⁹²═—H and A⁴=—P(═O) (OR³¹⁶)(OR³¹⁷) and R³¹⁶═—C₆H₅ and R³¹⁷═—C₆H₅, namely glutaminyl-(2,5-dihydro-1H-pyrrole-2-phosphonic acid diphenyl ester).

(510) Compound according to general formula (I) containing L-α-glutamine or L-α-homoglutamine, wherein X⁴═CR²⁹¹ and R²⁹¹═CH₃ and X⁵═CR²⁹² and R²⁹²═—H and A⁴=—C≡N, namely glutaminyl-(4-methyl-2,5-dihydro-1H-pyrrole-2-carbonitrile).

(511) Compound according to general formula (I) containing L-α-glutamine or L-α-homoglutamine, wherein X⁴═CR²⁹¹ and R²⁹¹═—C₆H₅ and X¹═CR²⁹² and R²⁹²═—H and A⁴═C≡N, namely glutaminyl-(4-phenyl-2,5-dihydro-1H-pyrrole-2-carbonitrile).

(512) Compound according to general formula (I) containing L-α-glutamine or L-α-homoglutamine, wherein X⁴═CR²⁹¹ and R²⁹¹═CF₃ and X⁵═CR²⁹² and R²⁹²═—H and A⁴=—C≡N, namely glutaminyl-(4-trifluoromethyl-2,5-dihydro-1H-pyrrole-2-carbonitrile).

(513) Compound according to general formula (I) containing L-α-glutamine or L-αhomoglutamine, wherein X⁴═CR²⁹¹ and R²⁹¹═—H and X⁵═CR²⁹² and R²⁹²═CH₃ and A⁴=—C≡N, namely glutaminyl-(3-methyl-2,5-dihydro-1H-pyrrole-2-carbonitrile).

(514) Compound according to general formula (I) containing L-α-glutamine or L-α-homoglutamine, wherein X⁴═CR²⁹¹ and R²⁹¹═—H and X⁵═CR²⁹² and R²⁹²═—C H₅ and A⁴=—C≡N, namely glutaminyl-(3-phenyl-2,5-dihydro-1H-pyrrole-2-carbonitrile).

(515) Compound according to general formula (I) containing L-α-glutamine or L-αhomoglutamine, wherein X⁴═CR²⁹¹ and R²⁹¹═—H and X⁵═CR^{292 and R}²⁹²═CF₃ and A⁴=—C—N, namely glutaminyl-(3-trifluoromethyl-2,5-dihydro-1H-pyrrole-2-carbonitrile).

(516) Compound according to general formula (I) containing L-α-glutamine or L-α-homoglutamine, wherein X⁴═CR²⁹¹ and R²⁹¹═CH₃ and X⁵═CR²⁹² and R²⁹²═CH₃ and A⁴═C—N, namely glutaminyl-(3,4-dimethyl-2,5-dihydro-1H-pyrrole-2-carbonitrile).

(517) Compound according to general formula (I) containing L-α-glutamine or L-αhomoglutamine, wherein X⁴═CR²⁹¹ and R²⁹¹═CH₃ and X⁵═CR^{292 and R}²⁹²═—H and A⁴=COOH, namely glutaminyl-(4-methyl-2,5-dihydro-1H-pyrrole-2-carboxylic acid).

(518) Compound according to general formula (I) containing L-α-glutamine or L-αhomoglutamine, wherein X⁴═CR²⁹¹ and R²⁹¹═—CrH and X¹═CR²⁹² and R²⁹²═—H and A⁴═COOH, namely glutaminyl-(4-phenyl-2,5-dihydro-1H-pyrrole-2-carboxylic acid).

(519) Compound according to general formula (I) containing L-α-glutamine or L-α-homoglutamine, wherein X⁴═CR²⁹¹ and R²⁹¹═CF₃ and X⁵═CR²⁹² and R²⁹²═—H and A⁴=—COOH, namely glutaminyl-(4-trifluoromethyl-2,5-dihydro-1H-pyrrole-2-carboxylic acid).

(520) Compound according to general formula (I) containing L-α-glutamine or L-α-homoglutamine, wherein X═CR²⁹¹ and R²⁹¹═—H and X═CR²⁹² and R²⁹²═CH₃ and A⁴=—COOH, namely glutaminyl-(3-methyl-2,5-dihydro-1H-pyrrole-2-carboxylic acid).

(521) Compound according to general formula (I) containing L-α-glutamine or L-αhomoglutamine, wherein X⁴═CR²⁹¹ and R²⁹¹═—H and X⁵═CR²⁹² and R²⁹²═—C₆H₅ and A⁴=—COOH, namely glutaminyl-(3-phenyl-2,5-dihydro-1H-pyrrole-2-carboxylic acid).

(522) Compound according to general formula (I) containing L-α-glutamine or L-αhomoglutamine, wherein X⁴═CR²⁹¹ and R²⁹¹═—H and X⁵═CR²⁹² and R²⁹²═CF₃ and A⁴=—COOH, namely glutaminyl-(3-tiifluoromethyl-2,5-dihydro-1H-pyrrole-2-carboxylic acid).

(523) Compound according to general formula (I) containing L-α-glutamine or L-αhomoglutamine, wherein X⁴═CR²⁹¹ and R²⁹¹═CH₃ and X⁵═CR²⁹² and R²⁹²═CH₃ and A⁴=—COOH, namely glutaminyl-(3,4-dimethyl-2,5-dihydro-1H-pyrrole-2-carboxylic acid).

(524) Compound according to general formula (I) containing L-α-glutamine or L-α-homoglutamine, wherein X⁴═CR²⁹¹ and R²⁹¹═CH₃ and X⁵═CR²⁹² and R²⁹²═—H and A⁴=—B(OH)₂, namely glutaminyl-(4-methyl-2,5-dihydro-1H-pyrrole-2-boronic acid).

(525) Compound according to general formula (I) containing L-α-glutamine or L-α-homoglutamine, wherein X⁴═CR²⁹¹ and R²⁹¹═—C₆H₅ and X⁵═CR²⁹² and R²⁹²═—H and A⁴=—B(OH)₂, namely glutaminyl-(4-phenyl-2,5-dihydro-1H-pyrrole-2-boronic acid).

(526) Compound according to general formula (I) containing L-α-glutamine or L-α-homoglutamine, wherein X⁴═CR²⁹¹ and R²⁹¹═CF₃ and X⁵═CR²⁹² and R²⁹²═—H and A⁴=—B(OH)₂, namely glutaminyl-(4-trifluoromethyl-2,5-dihydro-1H-pyrrole-2-boronic acid).

(527) Compound according to general formula (I) containing L-α-glutamine or L-α-homoglutamine, wherein X⁴═CR²⁹¹ and R²⁹¹═—H and X⁵═CR²⁹² and R²⁹²═CH₃ and A⁴=—B(OH)₂, namely glutaminyl-(3-methyl-2,5-dihydro-1H-pyrrole-2-boronic acid).

(528) Compound according to general formula (I) containing L-α-glutamine or L-α-homoglutamine, wherein X⁴═CR²⁹ and R²⁹¹═—H and X⁵═CR²¹² and R²¹²═—C₆H₅ and A⁴=B(OH)₂, namely glutaminyl-(3-phenyl-2,5-dihydro-1H-pyrrole-2-boronic acid).

(529) Compound according to general formula (I) containing L-α-glutamine or L-α-homoglutamine, wherein X⁴═CR²⁹¹ and R²⁹¹═—H and X⁵═CR²⁹² and R²⁹²═CF₃ and A⁴=—B(OH)₂, namely glutaminyl-(3-trifluoromethyl-2,5-dihydro-1H-pyrrole-2-boronic acid).

(530) Compound according to general formula (I) containing L-α--glutanine or L-α-homoglutamine, wherein X⁴═CR²¹ and R²¹¹═CH₃ and X¹═CR²⁹² and R²⁹²═CH₃ and A⁴=B(OH)₂, namely glutaminyl-(3,4-dimethyl-2,5-dihydro-1H-pyrrole-2-boronic acid).

(531) Compound according to general formula (I) containing L-α-glutamine or L-α-homoglutamine, wherein X⁴═CR²⁹ and R²⁹¹═CH₃ and X⁵═CR²⁹² and R²¹²═—H and A⁴=—P(═O) (OR³¹⁶)(OR³¹⁷) and R³¹⁶═—C₆H₅ and R³¹⁷═—C₆H₅, namely glutaminyl-(4-methyl-2,5-dihydro-1H-pyrrole-2-phosphonic acid diphenyl ester).

(532) Compound according to general formula (I) containing L-α-glutamine or L-α-homoglutamine, wherein X⁴═CR²⁹¹ and R²⁹¹═—C₆H₅ and X⁵═CR²⁹² and R²⁹²═—H and A⁴=—P(═O) (OR³¹⁶)(OR³¹⁷) and R³¹⁶═—C₆H₅ and R³¹⁷═—C₆H₅, namely glutaminyl-(4-phenyl-2,5-dihydro-1H-pyrrole-2-phosphonic acid diphenyl ester).

(533) Compound according to general formula (I) containing L-α-glutamine or L-αhomoglutamine, wherein X═CR²⁹¹ and R²⁹¹═CF₃ and X═CR²⁹² and R²⁹²═—H and A⁴=—P(═O) (OR³¹⁶)(OR³¹⁷) and R³¹⁶═—C₆H₅ and R³¹⁷═—C₆H₅, namely glutaminyl-(4-trifluoromethyl-2,5-dihydro-1H-pyrrole-2-phosphonic acid diphenyl ester).

(534) Compound according to general formula (I) containing L-α-glutamine or L-α-homoglutamine, wherein X⁴═CR²⁹¹ and R²⁹¹═—H and X⁵═CR²⁹² and R²⁹²═CH₃ and A⁴=—P(═O)(OR³¹⁶)(OR³¹⁷) and R³¹⁶═—C₆H₅ and R³¹⁷═—C₆H₅, namely glutaminyl-(3-methyl-2,5-dihydro-1H-pyrrole-2-phosphonic acid diphenyl ester).

(535) Compound according to general formula (I) containing L-α-glutamine or L-α-homoglutamine, wherein X⁴═CR²⁹¹ and R²⁹¹═—H and X⁵═CR²⁹² and R²⁹²═—C₆H₅ and A⁴=—P(═O) (OR³¹⁶)(OR³¹⁷) and R³¹⁶═—C₆H₅ and R³¹⁷═—C₆H₅, namely glutaminyl-(3-phenyl-2,5-dihydro-1H-pyrrole-2-phosphonic acid diphenyl ester).

(536) Compound according to general formula (I) containing L-α-glutamine or L-α-homoglutamine, wherein X⁴═CR²⁹⁰ and R²⁹¹═—H and X¹═CR²⁹² and R²⁹²═CF₃ and A⁴=—P(═O) (OR³¹⁶)(OR³¹⁷) and R³¹¹═—C₆H₅ and R³¹⁷═—C₆H₅, namely glutaminyl-(3-trifluoromethyl-2,5-dihydro-1H-pyrrole-2-phosphonic acid diphenyl ester).

(537) Compound according to general formula (I) containing L-α-glutamine or L-α-homoglutamine, wherein X⁴═CR²⁹¹ and R²⁹¹═CH₃ and X⁵═CR²⁹² and R²⁹²═CH₃ and A⁴=—P(═O) (OR³¹⁶)(OR³¹⁷) and R³¹⁶═—C₆H₅ and R³¹⁷═—C₆H₅, namely glutaminyl-(3,4-dimethyl-2,5-dihydro-1H-pyrrole-2-phosphonic acid diphenyl ester).

(538) Compound according to general formula (I) containing L-α-glutamine or L-α-homoglutamine, wherein X⁴═N and X⁵═CR²⁹² and R²⁹²═—H and A⁴=—H, namely glutaminyl-(1N-2,5-dihydro-1H-imidazole).

(539) Compound according to general formula (I) containing L-α-glutamine or L-α-homoglutamine, wherein X⁴═N and X⁵═CR^{292 and R}²⁹²═—H and A⁴═—CN, namely glutaminyl-(1 N-2,5-dihydro-1H-imidazole-5-carbonitrile).

(540) Compound according to general formula (I) containing L-α-glutamine or L-α-homoglutamine, wherein X⁴=N and X⁵═CR²⁹² and R²⁹²═—H and A⁴=—COOH, namely glutaminyl-(1N-2,5-dihydro-1H-imidazole-5-carboxylic acid).

(541) Compound according to general formula (I) containing L-α-glutamine or L-α-homoglutamine, wherein X⁴=N and X⁵═CR²⁹² and R²⁹²═—H and A⁴=—CONH₂, namely glutaminyl-(1N-2,5-dihydro-1H-imidazole-5-carboxamide).

(542) Compound according to general formula (I) containing L-α-glutamine or L-α-homoglutamine, wherein X⁴=N and X⁵═CR²⁹² and R²⁹²═—H and A⁴=—B(OH)₂, namely glutaminyl-(1N-2,5-dihydro-1H-imidazole-5-boronic acid).

(543) Compound according to general formula (I) containing L-α-glutamine or L-α-homoglutamine, wherein X⁴=N and X⁵═CR²⁹² and R²⁹²═—H and A⁴=—SO₃H, namely glutaminyl-(1N-2,5-dihydro-1H-imidazole-5-sulfonic acid).

(544) Compound according to general formula (I) containing L-α-glutamine or L-α-homoglutamine, wherein X⁴═N and X⁵═CR²⁹² and R²⁹²═—H and A⁴=—P(═O) (OR³¹⁶)(OR³¹⁷) and R³¹⁶═—C₆H₅ and R³¹⁷═—C₆H₅, namely glutaminyl-(1N-2,5-dihydro-1H-imidazole-5-phosphonic acid diphenyl ester).

(545) Compound according to general formula (I) containing L-α-glutamine or L-α-homoglutamine, wherein X⁴═CR²⁹ and R²⁹¹═—H and X⁵═N and A⁴=—C≡N, namely glutaminyl-(1N-2,5-dihydro-1H-imidazole-2-carbonitrile).

(546) Compound according to general formula (I) containing L-α-glutamine or L-α-homoglutamine, wherein X⁴═CR²⁹¹ and R²⁹¹═—H and X⁵═N and A⁴=—COOH, namely glutaminyl-(1N-2,5-dihydro-1H-imidazole-2-carboxylic acid).

(547) Compound according to general formula (I) containing L-α-glutamine or L-α-homoglutamine, wherein X⁴═CR²⁹¹ and R²⁹¹═—H and X⁵═N and A⁴=—CONH₂, namely glutaminyl-(1N-2,5-dihydro-1H-imidazole-2-carboxamide).

(548) Compound according to general formula (I) containing L-α-glutamine or L-α-homoglutamine, wherein X⁴═CR²⁹ and R²⁹¹═—H and X⁵=N and A⁴=—B(OH)₂, namely glutaminyl-(1N-2,5-dihydro-1H-imidazole-2-boronic acid).

(549) Compound according to general formula (I) containing L-α-glutamine or L-α-homoglutamine, wherein X⁴═CR²⁹ and R²⁹¹═—H and X⁵=N and A⁴=—SO₃H, namely glutaminyl-(1N-2,5-dihydro-1H-imidazole-2-sulfonic acid).

(550) Compound according to general formula (I) containing L-α-glutamine or L-α-homoglutamine, wherein X⁴═CR²⁹¹ and R²⁹¹═—H and X⁵=N and A⁴=—P(═O) (OR³¹⁶)(OR⁷) and R¹¹⁶═—C₆H₅ and R³¹⁷═—C₆H₅, namely glutaminyl-(1N-2,5-dihydro-1H-imidazole-2-phosphonic acid diphenyl ester).

(551) Compound according to general formula (I) containing L-α-glutamine or L-α-homoglutamine, wherein X⁴═CR²⁹ and R²⁹¹═—CF₃ and X⁵=N and A⁴=—H, namely glutaminyl-(4-trifluoromethyl-2,5-dihydro-1H-imidazole).

(552) Compound according to general formula (I) containing L-α-glutamine or L-α-homoglutamine, wherein X⁴═CR²⁹¹ and R²⁹¹═—CF₃ and X⁵=N and A⁴=—C≡N, namely glutaminyl-(4-trifluoromethyl-2,5-dihydro-1H-imidazole-2-carbonitrile).

(553) Compound according to general formula (I) containing L-α-glutamine or L-α-homoglutamine, wherein X⁴═CR⁹¹ and R²⁹¹═—CF₃ and X⁵=N and A⁴=—B(OH)₂, namely glutaminyl-(4-trifluoromethyl-2,5-dihydro-1H-imidazole-2-boronic acid).

(554) Compound according to general formula (I) containing L-α-glutamine or L-α-homoglutamine, wherein X⁴═CR⁹¹ and R²⁹¹═—CF₃ and X⁵=N and A⁴=—P(═O) (OR³⁶)(OR³⁷) and R³¹⁶═—C₆H₅ and R³¹⁷═—C₆H₅, namely glutaminyl-(4-trifluoromethyl-2,5-dihydro-1H-imidazole-2-phosphonic acid diphenyl ester).

(555) Compound according to general formula (I) containing L-α-glutamine or L-α-homoglutamine, wherein X⁴=N and X⁵═CR²⁹² and R²⁹²═—CF₃ and A 4═—C≡N, namely glutaminyl-(4-trifluoromethyl-2,5-dihydro-1H-imidazole-5-carbonitrile).

(556) Compound according to general formula (I) containing L-α-glutamine or L-α-homoglutamine, wherein X⁴=N and X⁵═CR²⁹² and R²⁹²═—CF₃ and A⁴=—B(OH)₂, namely glutaminyl-(4-trifluoromethyl-2,5-dihydro-1H-imidazole-5-boronic acid).

(557) Compound according to general formula (I) containing L-α-glutamine or L-α-homoglutamine, wherein X⁴=N and X⁵═CR²⁹² and R²⁹²═—CF₃ and A⁴=—P(═O) (OR³¹⁶)(OR³¹⁷) and R³¹⁶═—C₆H₅ and R³¹⁷═—C₆H₅, namely glutaminyl-(4-trifluoromethyl-2,5-dihydro-1H-imidazole-5-phosphonic acid diphenyl ester).

(558) Compound according to general formula (I) containing L-α-glutamine or L-α-homoglutamine, wherein X⁴═N and X⁵═N and A⁴=—H, namely glutaminyl-(4N-3,5-dihydro-4H-1,2,4-triazole).

(559) Compound according to general formula (I) containing L-α-glutamine or L-α-homoglutamine, wherein X⁴═N and X⁵═N and A⁴=—C≡N, namely glutaminyl-(4N-3,5-dihydro-4H-1,2,4-triazole-3-carbonitrile).

(560) Compound according to general formula (I) containing L-α-glutamine or L-α-homoglutamine, wherein X⁴═N and X⁵═N and A⁴=—COOH, namely glutaminyl-(4N-3,5-dihydro-4H-1,2,4-triazole-3-carboxylic acid).

(561) Compound according to general formula (I) containing L-α-glutarine or L-α-homoglutamine, wherein X⁴═N and X⁵═N and A⁴=—CO—NH₂, namely glutaminyl-(4N-3,5-dihydro-4H-1,2,4-triazole-3-carboxamide).

(562) Compound according to general formula (I) containing L-α-glutamine or L-α-homoglutamine, wherein X⁴═N and X⁵═N and A⁴=—B(OH)₂, namely glutaminyl-(4N-3,5-dihydro-4H-1,2,4-triazole-3-boronic acid).

(563) Compound according to general formula (I) containing L-α-glutamine or L-α-homoglutamine, wherein X⁴═N and X⁵═N and A⁴=—P(═O) (OH)₂, namely glutaminyl-(4N-3,5-dihydro-4H-1,2,4-triazole-3-phosphonic acid).

(564) Compound according to general formula (I) containing L-α-glutamine or L-α-homoglutamine, wherein X⁴═N and X⁵═N and A⁴=—P(═O) (OR³¹⁶)(OR³¹⁷) and R³¹⁶═—C₆H₅ and R³¹⁷═—C₆H₅, namely glutaminyl-(4N-3,5-dihydro-4H-1,2,4-triazole-3-phosphonic acid diphenyl ester).

Examples for Prolin Mimetics of Formula (VI):

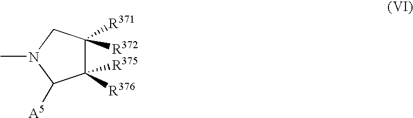

(600) Compound according to general formula (I) containing L-α-glutamine or L-α-homoglutamine, wherein R³⁷¹═F and R³⁷²═H and R³⁷⁵═H and R³⁷⁶═H and A═—H, namely glutaminyl-(3R-fluoro-pyrrolidine).

(601) Compound according to general formula (I) containing L-α-glutamine or L-α-homoglutamine, wherein R³⁷¹═H and R³⁷²═F and R³⁷¹═H and R³⁷⁶═H and A⁵=—H, namely glutaminyl-(3S-fluoro-pyrrolidine).

(602) Compound according to general formula (I) containing L-α-glutamine or L-α-homoglutamine, wherein R³⁷¹═F and R³⁷²═F and R³⁷═H and R³⁷⁶═H and A═—H, namely glutaminyl-(3,3-difluoro-pyrrolidine).

(603) Compound according to general formula (I) containing L-α-glutamine or L-α-homoglutamine, wherein R³⁷¹═F and R³⁷²═H and R³⁷⁵═F and R³⁷⁶═H and A⁵=—H, namely glutaminyl-(meso-3,4-difluoro-pyrrolidine).

(604) Compound according to general formula (I) containing L-α-glutamine or L-α-homoglutamine, wherein R³¹¹═F and R³⁷²═H and R³⁷⁵═H and R¹⁷⁶═F and A⁵=—H, namely glutaminyl-(3S,4S-difluoro-pyrrolidine).

(605) Compound according to general formula (I) containing L-α-glutamine or L-α-homoglutamine, wherein R³⁷═H and R³⁷²═F and R³⁷═F and R¹⁷⁶═H and A⁵=—H, namely glutaminyl-(3R,4R-difluoro-pyrrolidine).

(606) Compound according to general formula (I) containing L-α-glutamine or L-α-homoglutamine, wherein R³⁷¹═—OH and R³⁷²═H and R³⁷⁵═H and R³⁷⁶═H and A⁵=—H, namely glutaminyl-(3R-hydroxy-pyrrolidine).

(607) Compound according to general formula (I) containing L-α-glutamine or L-α-homoglutamine, wherein R³⁷¹═H and R³⁷²═—OH and R³⁷⁵═H and R³⁷⁶═H and A⁵=—H, namely glutaminyl-(3S-hydroxy-pyrrolidine).

(608) Compound according to general formula (I) containing L-α-glutamine or L-α-homoglutamine, wherein R³⁷¹+R³⁷²═(═O) and R³⁷═H and R³⁷⁶═H and A═—H, namely glutaminyl-(3-oxo-pyrrolidine).

(609) Compound according to general formula (I) containing L-α-glutamine or L-α-homoglutamine, wherein R³⁷═F and R³⁷²═H and R³⁷═H and R³⁷¹═H and A═—C_N, namely glutaminyl-(4R-fluoro-pyrrolidine-2S-carbonitrile).

(610) Compound according to general formula (I) containing L-α-glutamine or L-α-homoglutamine, wherein R³⁷═H and R³⁷²═F and R³⁷═H and R¹⁷¹═H and A¹=—C≡N, namely glutaminyl-(4S-fluoro-pyrrolidine-2S-carbonitrile).

(611) Compound according to general formula (I) containing L-α-glutamine or L-α-homoglutamine, wherein R³⁷═F and R³⁷²═F and R³⁷═H and R³⁷¹═H and A¹=—C≡N namely glutaminyl-(4,4-difluoro-pyrrolidine-2-carbonitrile).

(612) Compound according to general formula (I) containing L-α-glutamine or L-α-homoglutamine, wherein R³⁷¹═H and R³⁷²═H and R³⁷⁵═F and R³⁷⁶═H and A⁵=—C≡N, namely glutaminyl-(3S-fluoro-pyrrolidine-2S-carbonitrile).

(613) Compound according to general formula (I) containing L-α-glutamine or L-α-homoglutamine, wherein R³⁷¹═F and R³⁷²═H and R³⁷⁵═F and R³⁷⁶═H and A⁵=—C≡N, namely glutaminyl-(3S,4R-difluoro-pyrrolidine-2S-carbonitrile).

(614) Compound according to general formula (I) containing L-α-glutamine or L-α-homoglutamine, wherein R³⁷═H and R³⁷²═F and R³⁷⁵═F and R³⁷⁶═H and A⁵=—C≡N, namely glutaminyl-(3S,4S-difluoro-pyrrolidine-2S-carbonitrile).

(615) Compound according to general formula (I) containing L-α-glutamine or L-α-homoglutamine, wherein R³⁷¹═H and R³⁷²═H and R³⁷⁵═H and R³⁷⁶═F and A¹=—C≡N, namely glutaminyl-(3R-fluoro-pyrrolidine-2S-carbonitrile) (Epimer zu 197).

(616) Compound according to general formula (I) containing L-α-glutamine or L-α-homoglutamine, wherein R³⁷¹═H and R³⁷²═F and R³⁷¹═H and R³⁷⁶═F and A⁵=—C≡N namely glutaminyl-(3R,4R-fluoro-pyrrolidine-2S-carbonitrile) (Epimer zu 197).

(617) Compound according to general formula (I) containing L-α-glutamine or L-α-homoglutamine, wherein R³⁷═F and R³⁷²═H and R³⁷¹═H and R³⁷⁶═F and A¹=—C≡N, namely glutaminyl-(3R,4S-fluoro-pyrrolidine-2S-carbonitrile) (Epimer zu 197).

(618) Compound according to general formula (I) containing L-α-glutamine or L-α-homoglutamine, wherein R³⁷¹═—H and R³⁷²═—H and R³⁷⁵═—F and R³⁷⁶═—F and A¹=—C≡N, namely glutaminyl-(3,3-difluoro-pyrrolidine-2S-carbonitrile).

(619) Compound according to general formula (I) containing L-α-glutamine or L-αhomoglutamine, wherein R³⁷¹=—F and R³⁷²=—F and R³⁷⁵=—F and R³⁷⁶=—F and A═—C≡N, namely glutaminyl-(3,3,4,4,-tetrafluoro-pyrrolidine-2S-carbonitrile).

(620) Compound according to general formula (I) containing L-α-glutamine or L-α-homoglutamine, wherein R³⁷¹═F and R³⁷²═H and R³⁷⁵═H and R³⁷⁶═H and A¹=—COOH, namely glutaminyl-(4R-fluoro-pyrrolidine-2S-carboxylic acid).

(621) Compound according to general formula (I) containing L-α-glutamine or L-α-homoglutamine, wherein R³⁷¹═H and R³⁷²═F and R³⁷⁵═H and R³⁷⁶═H and A⁵=—COOH, namely glutaminyl-(4S-fluoro-pyrrolidine-2S-carboxylic acid).

(622) Compound according to general formula (I) containing L-α-glutamine or L-α-homoglutamine, wherein R³⁷¹═F and R³⁷²═F and R³⁷⁵═H and R³⁷⁶═H and A⁵=—COOH, namely glutaminyl-(4,4-difluoro-pyrrolidine-2-carboxylic acid).

(623) Compound according to general formula (I) containing L-α-glutamine or L-α-homoglutamine, wherein R³⁷¹═H and R³⁷²═H and R³⁷⁵═F and R³⁷⁶═H and A⁵=—COOH, namely glutaminyl-(3S-fluoro-pyrrolidine-2S-carboxylic acid).

(624) Compound according to general formula (I) containing L-α-glutamine or L-α-homoglutamine, wherein R³⁷¹═F and R³⁷²═H and R³⁷¹═F and R³⁷⁶═H and A⁵=—COOH, namely glutaminyl-(3S,4R-difluoro-pyrrolidine-2S-carboxylic acid).

(625) Compound according to general formula (I) containing L-α-glutamine or L-α-homoglutamine, wherein R³⁷¹═H and R³⁷²═F and R³⁷⁵═F and R³⁷⁶═H and A⁵=—COOH, namely glutaminyl-(3S,4S-difluoro-pyrrolidine-2S-carboxylic acid).

(626) Compound according to general formula (I) containing L-α-glutamine or L-α-homoglutamine, wherein R³⁷¹═H and R³⁷²═H and R³⁷⁵═H and R³⁷⁶═F and A⁵=—COOH, namely glutaminyl-(3R-fluoro-pyrrolidine-2S-carboxylic acid).

(627) Compound according to general formula (I) containing L-α-glutamine or L-α-homoglutamine, wherein R³⁷¹═H and R³⁷²═F and R³⁷⁵═H and R³⁷⁶═F and A⁵=—COOH, namely glutaminyl-(3R,4R-fluoro-pyrrolidine-2S-carboxylic acid).

(628) Compound according to general formula (I) containing L-α-glutamine or L-α-homoglutamine, wherein R³⁷¹═F and R³⁷²═H and R³⁷⁵═H and R³⁷⁶═F and A⁵=—COOH, namely glutaminyl-(3R,4S-fluoro-pyrrolidine-2S-carboxylic acid).

(629) Compound according to general formula (I) containing L-α-glutamine or L-α-homoglutamine, wherein R³¹ ═—H and R³⁷²═—H and R³¹═—F and R¹⁷⁶═—F and A¹=—COOH, namely glutaminyl-(3,3-difluoro-pyrrolidine-2S-carboxylic acid).

(630) Compound according to general formula (I) containing L-α-glutamine or L-α-homoglutamine, wherein R³⁷¹═—F and R³⁷²═—F and R³⁷⁵═—F and R³⁷⁶═—F and A⁵=—COOH, namely glutaminyl-(3,3,4,4,-tetrafluoro-pyrrolidine-2S-carboxylic acid).

(631) Compound according to general formula (I) containing L-α-glutamine or L-α-homoglutamine, wherein R³⁷¹═F and R³⁷²═H and R³⁷⁵═H and R³⁷¹═H and A¹=—B(OH)₂, namely glutaminyl-(4R-fluoro-pyrrolidine-2S-boronic acid).

(632) Compound according to general formula (I) containing L-α-glutamine or L-α-homoglutamine, wherein R³⁷¹═H and R³⁷²═F and R³⁷⁵═H and R³⁷⁶═H and A⁵=—B(OH)₂, namely glutaminyl-(4S-fluoro-pyrrolidine-2S-boronic acid).

(633) Compound according to general formula (I) containing L-α-glutamine or L-α-homoglutamine, wherein R³⁷¹═F and R³⁷²═F and R³⁷⁵═H and R³⁷⁶═H and A¹=—B(OH)₂, namely glutaminyl-(4,4-difluoro-pyrrolidine-2-boronic acid).

(634) Compound according to general formula (I) containing L-α-glutamine or L-α-homoglutamine, wherein R³⁷¹═H and R³⁷²═H and R³⁷⁵═F and R³⁷⁶═H and A⁵=—B(OH)₂, namely glutaminyl-(3S-fluoro-pyrrolidine-2S-boronic acid).

(635) Compound according to general formula (I) containing L-α-glutamine or L-α-homoglutamine, wherein R³⁷¹═F and R³⁷²═H and R³¹¹═F and R³⁷⁶═H and A¹=—B(OH)₂, namely glutaminyl-(3S,4R-difluoro-pyrrolidine-2S-boronic acid).

(636) Compound according to general formula (I) containing L-α-glutamine or L-α-homoglutamine, wherein R³⁷¹═H and R³⁷²═F and R³⁷¹═F and R³⁷⁶═H and As=—B(OH)₂, namely glutaminyl-(3S,4S-difluoro-pyrrolidine-2S-boronic acid).

(637) Compound according to general formula (I) containing L-(X-glutamine or L-α-homoglutamine, wherein R³⁷¹═H and R³⁷²═H and R³⁷⁵═H and R³⁷⁶═F and A⁵=—B(OH)₂, namely glutaminyl-(3R-fluoro-pyrrolidine-2S-boronic acid).

(638) Compound according to general formula (I) containing L-α-glutamine or L-α-homoglutamine, wherein R³⁷¹═H and R³⁷²═F and R³⁷¹═H and R³⁷⁶═F and A⁵=—B(OH)₂, namely glutaminyl-(3R,4R-fluoro-pyrrolidine-2S-boronic acid).

(639) Compound according to general formula (I) containing L-α-glutamine or L-α-homoglutamine, wherein R³⁷¹═F and R³⁷²═H and R³⁷⁵═H and R³⁷⁶═F and A¹=—B(OH)₂, namely glutaminyl-(3R,4S-fluoro-pyrrolidine-2S-boronic acid).

(640) Compound according to general formula (I) containing L-α-glutamine or L-αhomoglutamine, wherein R³⁷¹═—H and R³⁷²═—H and R³⁷⁵═—F and R³⁷⁶═—F and A¹=—B(OH)₂, namely glutaminyl-(3,3-difluoro-pyrrolidine-2S-boronic acid).

(641) Compound according to general formula (I) containing L-α-glutamine or L-α-homoglutamine, wherein R³⁷¹═—F and R³⁷²═—F and R³⁷s=—F and R³¹⁶═—F and A⁵=—B(OH)₂, namely glutaminyl-(3,3,4,4-tetrafluoro-pyrrolidine-2S-boronic acid).

(642) Compound according to general formula (I) containing L-α-glutamine or L-α-homoglutamine, wherein R³⁷¹═F and R³⁷²═H and R³⁷⁵═H and R³⁷⁶═H and A⁵=—P(═O) (OR³⁹⁶)(OR³⁹⁷) and R³⁹⁶═—C₆H₅ and R³⁹⁷═—C₆H₅, namely glutaminyl-(4R-fluoro-pyrrolidine-2S-phosphonic acid diphenyl ester).

(643) Compound according to general formula (I) containing L-α-glutamine or L-α-homoglutamine, wherein R³⁷¹═H and R³⁷²═F and R³⁷═H and R³⁷⁶═H and A¹=—P(═O) (OR³⁹⁶)(OR³⁹⁷) and R³⁹⁶═—C₆H₅ and R³⁹⁷═—C₆H₅, namely glutaminyl-(4S-fluoro-pyrrolidine-2S-phosphonic acid diphenyl ester).

(644) Compound according to general formula (I) containing L-α-glutamine or L-α-homoglutamine, wherein R³⁷¹═F and R³⁷²═F and R³⁷⁵═H and R³⁷⁶═H and A⁵=—P(═O) (OR³⁹⁶)(OR³⁹⁷) and R³⁹⁶═—C₆H₅ and R³⁹⁷═—C₆H₅, namely glutaminyl-(4,4-difluoro-pyrrolidine-2S-phosphonic acid diphenyl ester).

(645) Compound according to general formula (I) containing L-α-glutamine or L-α-homoglutamine, wherein R³⁷¹═H and R³⁷²═H and R³⁷═F and R³⁷⁶═H and A⁵=—P(═O) (OR³⁹⁶)(OR³⁹⁷) and R³⁹⁶═—C₆H₅ and R³⁹⁷═—C₆H₅, namely glutaminyl-(3S-fluoro-pyrrolidine-2S-phosphonic acid diphenyl ester).

(646) Compound according to general formula (I) containing L-α-glutamine or L-α-homoglutamine, wherein R³⁷¹═F and R³⁷²═H and R³⁷═F and R³⁷⁶═H and A⁵=—P(═O) (OR³⁹⁶)(OR³⁹⁷) and R³⁹⁶═—C₆H₅ and R³⁹⁷═—C₆H₅, namely glutaminyl-(3S,4R-difluoro-pyrrolidine-2S-phosphonic acid diphenyl ester).

(647) Compound according to general formula (I) containing L-α-glutamine or L-α-homoglutamine, wherein R³⁷¹═H and R³⁷²═F and R³⁷⁵=F and R³⁷⁶═H and A⁵=_p(═O) (OR³⁹⁶)(OR³⁹⁷) and R³⁹⁶═—C₆H₅ and R³⁹⁷═—C₆H₅, namely glutaminyl-(3S,4S-difluoro-pyrrolidine-2S-phosphonic acid diphenyl ester).

(648) Compound according to general formula (I) containing L-α-glutamine or L-α-homoglutamine, wherein R³⁷¹═H and R³⁷²═H and R³⁷⁵═H and R³⁷⁶═F and A⁵=—P(═O) (OR³⁹⁶)(OR³⁹⁷) and R³⁹⁶═—C₆H¹ and R³⁹⁷═—C₆H₅, namely glutaminyl-(3R-fluoro-pyrrolidine-2S-phosphonic acid diphenyl ester).

(649) Compound according to general formula (I) containing L-α-glutamine or L-α-homoglutamine, wherein R³⁷¹═H and R³⁷²═F and R³⁷⁵═H and R³⁷⁶═F and A⁵=—P(═O) (OR³⁹⁶)(OR³⁹⁷) and R³⁹⁶═—C₆H₅ and R³⁹⁷═—C₆H₅, namely glutaminyl-(3R,4R-fluoro-pyrrolidine-2S-phosphonic acid diphenyl ester).

(650) Compound according to general formula (I) containing L-α-glutamine or L-α-homoglutamine, wherein R³⁷¹═F and R³⁷²═H and R³⁷⁵═H and R³⁷⁶═F and A¹=—P(═O) (OR³⁹⁶)(OR³⁹⁷) and R³⁹⁶═—C₆H₅ and R³⁹⁷═—C₆H₅, namely glutaminyl-(3R,4S-fluoro-pyrrolidine-2S-phosphonic acid diphenyl ester).

(651) Compound according to general formula (I) containing L-α-glutamine or L-α-homoglutamine, wherein R³⁷¹═—H and R³⁷²═—H and R³⁷⁵═—F and R³⁷⁶═—F and A⁵=_p(═O) (OR³⁹⁶)(OR³⁹⁷) and R³⁹⁶═—C₆H₅ and R³⁹⁷═—C₆H₅, namely glutaminyl-(3,3-difluoro-pyrrolidine-2S-phosphonic acid diphenyl ester).

(652) Compound according to general formula (I) containing L-α-glutamine or L-αhomoglutamine, wherein R³⁷¹═—F and R³⁷²═—F and R³⁷⁵═—F and R³⁷⁶═—F and A⁵=—P(═O) (OR³⁹⁶)(OR³⁹⁷) and R³⁹⁶═—C₆H¹ and R³⁹⁷═—C₆H₅, namely glutaminyl-(3,3,4,4-tetrafluoro-pyrrolidine-2S-phosphonic acid diphenyl ester).

Examples for Prolin Mimetics of Formula (VII):

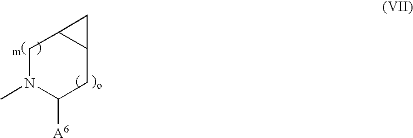

(700) Compound according to general formula (I) containing L-α-glutamine or L-α-homoglutamine, wherein m=0 and o=1 and A⁶=—H, namely 1-glutaminyl-(4,5-methano-25 pyrrolidine).

(701) Compound according to general formula (I) containing L-α-glutamine or L-α-homoglutamine, wherein m=1 and o=0 and A═—H, namely 1-glutaminyl-(3,4-methano-pyrrolidine).

(702) Compound according to general formula (I) containing L-α-glutamine or L-α-homoglutamine, wherein m=1 and o=1 and A═—H, namely 1-glutaminyl-(4,5-methano-piperidine).

(703) Compound according to general formula (I) containing L-α-glutamine or L-α-homoglutamine, wherein m=0 and o=2 and A═—H, namely 1-glutaminyl-(5,6-methano-piperidine).

(704) Compound according to general formula (I) containing L-α-glutamine or L-α-homoglutamine, wherein m=0 and o=1 and A⁶=—C≡N, namely 1-glutaminyl-(4,5-methano-pyrrolidin-2-carbonitrile).

(705) Compound according to general formula (I) containing L-α-glutamine or L-α-homoglutamine, wherein m=1 and o=0 and A⁶=—C≡N, namely 1-glutaminyl-(3,4-methano-pyrrolidin-2-carbonitrile).

(706) Compound according to general formula (I) containing L-α-glutamine or L-α-homoglutamine, wherein m=1 and o=1 and A═—C≡N, namely 1-glutaminyl-(4,5-methano-piperidin-2-carbonitrile).

(707) Compound according to general formula (I) containing L-α-glutamine or L-α-homoglutamine, wherein m=0 and o=2 and A═—C≡N, namely 1-glutaminyl-(5,6-methano-piperidin-2-carbonitrile).

(708) Compound according to general formula (I) containing L-α-glutamine or L-α-homoglutamine, wherein m=0 and o=1 and A⁶=—COOH, namely 1-glutaminyl-(4,5-methano-pyrrolidin-2-carboxylic acid).

(709) Compound according to general formula (I) containing L-α-glutamine or L-α-homoglutamine, wherein m=1 and o=0 and A⁶=—COOH, namely 1-glutaminyl-(3,4-methano-pyrrolidin-2-carboxylic acid).

(710) Compound according to general formula (I) containing L-α-glutamine or L-α-homoglutamine, wherein m=1 and o=1 and A⁶=—COOH, namely 1-glutaminyl-(4,5-methano-piperidin-2-carboxylic acid).

(711) Compound according to general formula (I) containing L-α-glutamine or L-α-homoglutamine, wherein m=0 and o=2 and A⁶=—COOH, namely 1-glutaminyl-(5,6-methano-piperidin-2-carboxylic acid).

(712) Compound according to general formula (I) containing L-α-glutamine or L-α-homoglutamine, wherein m=0 and o=1 and A⁶=—B(OH)₂, namely 1-glutaminyl-(4,5-methano-pyrrolidin-2-boronic acid).

(713) Compound according to general formula (I) containing L-α-glutamine or L-α-homoglutamine, wherein m=1 and o=0 and A⁶=—B(OH)₂, namely 1-glutaminyl-(3,4-methano-pyrrolidin-2-boronic acid).

(714) Compound according to general formula (I) containing L-α-glutamine or L-αhomoglutamine, wherein m=1 and o=1 and A⁶=—B(OH)₂, namely 1-glutaminyl-(4,5-methano-piperidin-2-boronic acid).

(715) Compound according to general formula (I) containing L-α-glutamine or L-α-homoglutamine, wherein m=0 and o=2 and A⁶=—B(OH)₂, namely 1-glutaminyl-(5,6-methano-piperidin-2-boronic acid).

(716) Compound according to general formula (I) containing L-α-glutamine or L-α-homoglutamine, wherein m=0 and o=1 and A⁶=—P(═O) (OR⁴⁷⁶)(OR⁴⁷⁷) and R⁴⁷⁷═—C₆H₅ and R⁴⁷⁷═—C₆H₅, namely 1-glutaminyl-(4,5-methano-pyrrolidin-2-phosphonic acid diphenyl ester).

(717) Compound according to general formula (I) containing L-α-glutamine or L-α-homoglutamine, wherein m=1 and o=0 and A⁶=_P(═O) (OR⁴⁷⁶)(OR⁴⁷⁷) and R⁴⁷⁶═—C₆H₅ and R⁴⁷⁷═—C₆H₅, namely 1-glutaminyl-(3,4-methano-pyrrolidin-2-phosphonic acid diphenyl ester).

(718) Compound according to general formula (I) containing L-α-glutamine or L-α-homoglutamine, wherein m=1 and o=1 and A⁶=—P(═O) (OR⁴⁷⁶)(OR⁴⁷⁷) and R⁴⁷⁶═—C₆H₅ and R⁴⁷⁷═—C₆H₅, namely 1-glutaminyl-(4,5-methano-piperidin-2-phosphonic acid diphenyl ester).

(715) Compound according to general formula (I) containing L-α-glutamine or L-α-homoglutamine, wherein m=0 and o=2 and A⁶=—P(═O) (OR⁴⁷⁶)(OR⁴⁷⁷) and R⁴⁷⁶═—C₆H₅ and R⁴⁷⁷═—C₆H₅, namely 1-glutaminyl-(5,6-methano-piperidin-2-phosphonic acid diphenyl ester).

Examples for Prolin Mimetics of Formula (VIII):

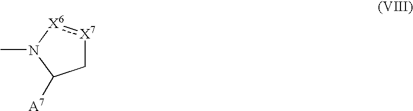

(800) Compound according to general formula (I) containing L-α-glutamine or L-α-homoglutamine, wherein X⁶═CR⁴⁹⁶ and R⁴⁹⁶═H and X⁷═CR⁴⁹⁷ and R⁴⁹⁷═H and X-X⁷=double bond and A⁷=—H, namely 1-glutaminyl-(2,3-dihydro-1H-pyrrole).

(801) Compound according to general formula (I) containing L-α-glutamine or L-α-homoglutamine, wherein X⁶=N and X⁷═CR⁴⁹⁷ and R⁴⁹⁷═H and X⁶-X⁷=double bond and A⁷ ═—H, namely 1-glutaminyl-(4,5-dihydro-1H-pyrazole).

(802) Compound according to general formula (I) containing L-α-glutamine or L-α-homoglutamine, wherein X⁶═CR⁴⁹⁶ and R⁴⁹⁶═H and X⁷=N and X⁶-X⁷=double bond and A⁷=—H, namely 1-glutaminyl-(4,5-dihydro-1H-imidazole).

(803) Compound according to general formula (I) containing L-α-glutamine or L-α-homoglutamine, wherein X⁶=N and X⁷═N and X⁶—X⁷=double bond and A⁷=—H, namely 1-glutaminyl-(4,5-dihydro-1H-1,2,3-triazole).

(804) Compound according to general formula (I) containing L-α-glutamine or L-α-20 homoglutamine, wherein x6=O and X⁷═CR⁴⁹³R⁴⁹⁴ and R⁴⁹³═H and R⁴⁹⁴═H and X⁶-X⁷=single bond and A⁷=—H, namely 2-glutaminyl-(isoxazolidine).

(805) Compound according to general formula (I) containing L-α-glutamine or L-α-homoglutamine, wherein X⁶═NR⁴⁹² and R⁴⁹²═H and X⁷═CR⁴⁹³R⁴⁹⁴ and R⁴⁹³═H and R⁴⁹⁴═H and X⁶—X⁷=single bond and A⁷=—H, namely 1-glutaminyl-(pyrazolidine).

(806) Compound according to general formula (I) containing L-α-glutamine or L-α-homoglutamine, wherein X⁶═CR⁴⁹⁶ and R⁴⁹⁶═H and X⁷═CR⁴⁹⁷ and R⁴═H and X⁶-X⁷=double bond and A⁷=—C≡N, namely 1-glutaminyl-(2,3-dihydro-1H-pyrrole-2-carbonitrile).

(807) Compound according to general formula (I) containing L-α-glutamine or L-α-homoglutamine, wherein X⁶═CR⁴⁹⁶ and R⁴⁹⁶═—C≡N and X⁷═CR⁴⁹⁷ and R⁴⁹⁷═H and X⁶—X⁷=double bond and A⁷=—H, namely 1-glutaminyl-(4,5-dihydro-1H-pyrrole-2-carbonitrile).

(808) Compound according to general formula (I) containing L-α-glutamine or L-α-homoglutamine, wherein X⁶═N and X⁷═CR⁴¹ and R⁴⁹⁷═H and X⁶-X⁷=double bond and A⁷ ═—C≡N, namely 1-glutaminyl-(4,5-dihydro-1H-pyrazole-5-carbonitrile).

(809) Compound according to general formula (I) containing L-α-glutamine or L-α-homoglutamine, wherein X⁶═CR⁴⁹⁶ and R⁴⁹⁶═H and X⁷═N and X⁶—X⁷=double bond and A⁷ ═—C≡N, namely 1-glutaminyl-(4,5-dihydro-1H-imidazole-5-carbonitrile).

(810) Compound according to general formula (I) containing L-α-glutamine or L-α-homoglutamine, wherein X⁶═CR⁴⁹⁶ and R⁴⁹⁶═—C≡N and X⁷═N and X⁶-X⁷=double bond and A⁷=—H, namely 1-glutaminyl-(4,5-dihydro-1H-imidazole-2-carbonitrile).

(811) Compound according to general formula (I) containing L-α-glutamine or L-α-homoglutamine, wherein X⁶=N and X⁷=N and X⁶-X⁷=double bond and A⁷=—C≡N, namely 1-glutaminyl-(4,5-dihydro-1H-1,2,3-triazole-5-carbonitrile).

(812) Compound according to general formula (I) containing L-α-glutamine or L-α-homoglutamine, wherein X⁶=O and X⁷═CR⁴⁹³R⁴⁹⁴ and R⁴⁹³═H and R⁴⁹⁴═H and X⁶-X⁷=single bond and A⁷=—C≡N, namely 2-glutaminyl-(isoxazolidine-3-carbonitrile).

(813) Compound according to general formula (I) containing L-α-glutamine or L-α-homoglutamine, wherein X⁶═NR⁴⁹² and R⁴⁹²═H and ═CR⁴⁹³R⁴⁹⁴ and R⁴⁹³═H and R⁴⁹⁴═H and X⁶-X⁷=single bond and A⁷=—C≡N, namely 1-glutaminyl-(pyrazolidine-5-carbonitrile).

(814) Compound according to general formula (I) containing L-α-glutamine or L-α-homoglutamine, wherein X⁶═NR⁴⁹² and R⁴⁹²═—C≡N and X⁷═CR⁴⁹³R⁴⁹⁴ and R⁴⁹³═H and R⁴⁹⁴ ═H and X^{6-X}⁷=single bond and A═H, namely 1-glutaminyl-(pyrazolidine-2N-carbonitrile).

(815) Compound according to general formula (I) containing L-α-glutamine or L-α-homoglutamine, wherein X⁶═CR¹⁶ and R⁴⁹⁶═H and X⁷═CR⁴⁹⁷ and R⁴⁹⁷═H and X⁶-X⁷=double bond and A⁷=—COOH, namely 1-glutaminyl-(2,3-dihydro-1H-pyrrole-2-carboxylic acid).

(816) Compound according to general formula (I) containing L-α-glutamine or L-α-homoglutamine, wherein X⁶═CR⁴⁹⁶ and R⁴⁹⁶═—COOH, and X⁷═CR⁴⁹⁷ and R⁴⁹⁷═H and X⁶—X⁷=double bond and A⁷=—H, namely 1-glutaminyl-(4,5-dihydro-1H-pyrrole-2-carboxylic acid).

(817) Compound according to general formula (1) containing L-α-glutamine or L-α-homoglutamine, wherein X⁶=N and X⁷═CR⁴⁹⁷ and R¹⁷═H and X⁶-X⁷=double bond and A⁷=—COOH, namely 1-glutaminyl-(4,5-dihydro-1H-pyrazole-5-carboxylic acid).

(818) Compound according to general formula (I) containing L-α-glutamine or L-α--homoglutamine, wherein X⁶═CR⁴⁹⁶ and R⁴⁹¹═H and X⁷=N and X⁶-X⁷=double bond and A⁷=—COOH, namely 1-glutaminyl-(4,5-dihydro-1H-imidazole-5-carboxylic acid).

(819) Compound according to general formula (I) containing L-α-glutamine or L-α-homoglutamine, wherein X⁶═CR⁴⁹⁶ and R⁴⁹⁶═—COOH and X⁷=N and X⁶—X⁷=double bond and A⁷=—H, namely 1-glutaminyl-(4,5-dihydro-1H-imidazole-2-carboxylic acid).

(820) Compound according to general formula (I) containing L-α-glutamine or L-α-homoglutamine, wherein X⁶=N and X⁷═N and X⁶—X⁷=double bond and A⁷=—COOH, namely 1-glutaminyl-(4,5-dihydro-1H-1,2,3-triazole-5-carboxylic acid).

(821) Compound according to general formula (I) containing L-α-glutamine or L-α-homoglutamine, wherein X⁶═O and X⁷═CR⁴⁹³R⁴⁹⁴ and R⁴⁹³═H and R⁴⁹⁴═H and X⁶—X⁷=single bond and A⁷=—COOH, namely 2-glutaminyl-(isoxazolidine-3-carboxylic acid).

(822) Compound according to general formula (I) containing L-α-glutamine or L-α-homoglutamine, wherein X⁶═NR⁴⁹² and R⁴⁹²═H and X⁷═CR⁴⁹³R⁴⁹⁴ and R⁴⁹³═H and R⁴⁹⁴═H and X⁶-X⁷=single bond and A⁷=—COOH, namely 1-glutaminyl-(pyrazolidine-5-carboxylic acid).

(823) Compound according to general formula (I) containing L-α-glutamine or L-α-homoglutamine, wherein X⁶═CR⁴⁹⁶ and R⁴⁹⁶═H and X⁷═CR⁴⁹⁷ and R⁴⁹⁷═H and X⁶-X⁷=double bond and A⁷=—B(OH)₂, namely 1-glutaminyl-(2,3-dihydro-1H-pyrrole-2-boronic acid).

(824) Compound according to general formula (I) containing L-α-glutamine or L-α-homoglutamine, wherein X⁶═CR⁴⁹⁶ and R⁴⁹⁶═—B(OH)₂ and X⁷═CR⁴⁹⁷ and R⁴⁹⁷═H and X⁶ —X⁷=double bond and A⁷=—H, namely 1-glutaminyl-(4,5-dihydro-1H-pyrrole-2-boronic acid).

(825) Compound according to general formula (I) containing L-α-glutamine or L-α-homoglutamine, wherein X⁶═N and X⁷═CR⁴⁹⁷ and R⁴⁹⁷═H and X⁶—X⁷=double bond and A⁷=—B(OH)₂, namely 1-glutaminyl-(4,5-dihydro-1H-pyrazole-5-boronic acid).

(826) Compound according to general formula (I) containing L-α-glutamine or L-α-homoglutamine, wherein X⁶═CR⁴⁹⁶ and R⁴⁹⁶═H and X⁷═N and X⁶-X⁷=double bond and A⁷=—B(OH)₂, namely 1-glutaminyl-(4,5-dihydro-1H-imidazole-5-boronic acid).

(827) Compound according to general formula (I) containing L-α-glutamine or L-α-homoglutamine, wherein X⁶═CR⁴⁹⁶ and R⁴⁹⁶═—B(OH)₂ and X⁷═N and X⁶—X⁷=double bond and A⁷=—H, namely 1-glutaminyl-(4,5-dihydro-1H-imidazole-2-boronic acid).

(828) Compound according to general formula (I) containing L-α-glutamine or L-α-homoglutamine, wherein X⁶=N and X⁷═N and X⁶—X⁷=double bond and A⁷=—B(OH)₂, namely 1-glutaminyl-(4,5-dihydro-1H-1,2,3-triazole-5-boronic acid).

(829) Compound according to general formula (I) containing L-α-glutamine or L-α-homoglutamine, wherein X⁶═O and X⁷═CR⁴⁹³R⁴⁹⁴ and R⁴⁹³═H and R⁴⁹⁴═H and X⁶—X⁷=single bond and A⁷=—B(OH)₂, namely 2-glutaminyl-(isoxazolidine-3-boronic acid).

(830) Compound according to general formula (I) containing L-α-glutamine or L-α-homoglutamine, wherein X⁶═NR¹⁹² and R¹⁹²═H and X⁷═CR⁴⁹³R⁴⁹⁴ and R⁴⁹³═H and R⁴⁹⁴═H and X⁶—X⁷=single bond and A⁷=—B(OH)₂, namely 1-glutaminyl-(pyrazolidine-5-boronic acid).

(831) Compound according to general formula (I) containing L-α-glutamine or L-α-homoglutamine, wherein X⁶=Ce6 and R⁴⁹⁶═H and X⁷═CR⁴⁹⁷ and R⁴⁹⁷═H and X⁶-X⁷=double bond and A⁷=—P(═O) (OR⁵⁵⁶)(OR⁵⁵⁷) and R⁵⁵⁶═—C₆H₅ and R⁵⁵⁷═—C₆H₅, namely 1-glutaminyl-(2,3-dihydro-1H-pyrrole-2-phosphonic acid diphenyl ester).

(832) Compound according to general formula (I) containing L-α-glutamine or L-α-homoglutamine, wherein X═CR⁴⁹⁶ and R⁴⁹⁶═-p(═O) (OR⁵¹⁶)(OR⁵¹⁷) and R⁵¹⁶═—C₆H₅ and R⁵¹⁷═—C₆H₅ and X⁷═CR⁴⁹⁷ and R⁴⁹⁷═H and X⁶-X⁷=double bond and A⁷=—H, namely 1-glutaminyl-(4,5-dihydro-1H-pyrrole-2-phosphonic acid diphenyl ester).

(833) Compound according to general formula (I) containing L-α-glutamine or L-α-homoglutamine, wherein X⁶=N and X⁷═CR⁴⁹⁷ and R⁴⁹⁷═H and X⁶-X⁷=double bond and A⁷=—P(═O) (OR⁵⁵⁶)(OR⁵⁵⁷) and R⁵⁵⁶═—C₆H₅ and R⁵⁵⁷═—C₆H₅, namely 1-glutaminyl-(4,5-dihydro-1H-pyrazole-5-phosphonic acid diphenyl ester).

(834) Compound according to general formula (I) containing L-α-glutamine or L-α-homoglutamine, wherein X⁶═CR⁴⁹⁶ and R⁴⁹⁶═H and X⁷═N and X⁶—X⁷=double bond and A⁷=—P(═O) (OR¹⁶)(OR¹⁷) and R⁵⁵⁶═—C₆H₅ and R⁵⁵⁷═—C₆H₅, namely 1-glutaminyl-(4,5-dihydro-1H-imidazole-5-phosphonic acid diphenyl ester).

(835) Compound according to general formula (I) containing L-α-glutamine or L-α-homoglutamine, wherein X⁶═CR⁴⁹⁶ and R⁴⁹⁶═—P(═O) (OR⁵¹⁶)(OR⁵¹⁷) and R⁵¹⁶═—C₆H₅ and R⁵¹⁷═—C₆H₅ and X⁷═N and X⁶-X⁷=double bond and A⁷=—H, namely 1-glutaminyl-(4,5-dihydro-1H-imidazole-2-phosphonic acid diphenyl ester).

(836) Compound according to general formula (I) containing L-α-glutamine or L-α-homoglutamine, wherein X⁶=N and X⁷═N and X⁶—X⁷=double bond and A⁷=—P(═O) (OR⁵⁵⁶)(OR⁵⁵⁷) and R⁵⁵⁶═—C₆H₅ and R⁵⁵⁷═—C₆H₅, namely 1-glutaminyl-(4,5-dihydro-1H-1,2,3-triazole-5-phosphonic acid diphenyl ester).

(837) Compound according to general formula (I) containing L-α-glutamine or L-α-homoglutamine, wherein X⁶═O and X⁷═CR⁴⁹³R⁴⁹⁴ and R⁴⁹³═H and R⁴⁹⁴═H and X⁶-X⁷=single bond and A⁷=—P(═O) (OR⁵⁵⁶)(OR⁵⁵⁷) and R⁵⁵⁶═—C₆H₅ and R⁵⁵⁷═—C₆H₅, namely 2-glutaminyl-(isoxazolidine-3-phosphonic acid diphenyl ester).

(838) Compound according to general formula (I) containing L-α-glutamine or L-α-homoglutamine, wherein X⁶═NR⁴⁹² and R⁴⁹²═H and X⁷═CR⁴⁹³R⁴⁹⁴ and R⁴⁹³═H and R⁴⁹⁴═H and X⁶-X⁷=single bond and A⁷=—P(═O) (OR⁵⁵⁶)(OR⁵⁵⁷) and R¹¹⁶═—C₆H₅ and R⁵⁵⁷═—C₆H₅, namely 1-glutaminyl-(pyrazolidine-5-phosphonic acid diphenyl ester).

Examples for Prolin Mimetics of Formula (IX):

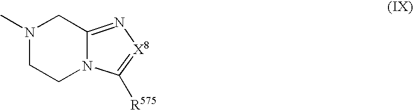

(900) Compound according to general formula (I) containing L-α-glutamine or L-αhomoglutamine, wherein X⁸═CR⁵⁷⁰ and R⁵⁷⁰═H and R⁵⁷⁵═H, namely 7-glutaminyl-(5,6,7,8-tetrahydro(imidazo[1,2-a]pyrazine).

(901) Compound according to general formula (I) containing L-α-glutamine or L-α-homoglutamine, wherein X⁸═CR⁵⁷⁰ and R⁵⁷⁰═—CH₃ and R⁵⁷⁵═H, namely 7-glutaminyl-(2-methyl-5,6,7,8-tetrahydro(imidazo[1,2-a]pyrazine).

(902) Compound according to general formula (I) containing L-α-glutamine or L-α-homoglutamine, wherein X⁸═CR⁵⁷⁰ and R⁵⁷⁰═—CF₃ and R⁵⁷⁵═H, namely 7-glutaminyl-(2-(trifluoromethyl)-5,6,7,8-tetrahydro(imidazo[1,2-a]pyrazine).

(903) Compound according to general formula (I) containing L-α-glutamine or L-α-homoglutamine, wherein X⁸═CR⁵⁷⁰ and R⁵⁷⁰═—CH₂CH₃ and R⁵⁷⁵═H, namely 7-glutaminyl-(2-ethyl-5,6,7,8-tetrahydro(imidazo[1,2-a]pyrazine).

(904) Compound according to general formula (I) containing L-α-glutamine or L-α-homoglutamine, wherein X⁸═CR⁵⁷⁰ and R⁵⁷¹═—C₆H₅ and R⁵⁷⁵═H, namely 7-glutaminyl-(2-phenyl-5,6,7,8-tetrahydro(imidazo[1,2-a]pyrazine).

(905) Compound according to general formula (I) containing L-α-glutamine or L-α-homoglutamine, wherein X⁸═CR⁵⁷⁰ and R⁵⁷⁰═-p-C₆H₅(CF₃) and R⁵⁷⁵═H, namely 7-glutaminyl-(2-(4-trifluoromethyl-phenyl)-5,6,7,8-tetrahydro(imidazo[1,2-a]pyrazine).

(906) Compound according to general formula (I) containing L-α-glutamine or L-α-homoglutamine, wherein X⁸═CR⁵⁷⁰ and R⁵⁷⁰═-p-C₆H₅(CF₂CF₃) and R⁵⁷⁵═H, namely 7-glutaminyl-(2-(4-pentafluoroethyl-phenyl)-5,6,7,8-tetrahydro(imidazo[1,2-a]pyrazine).

(907) Compound according to general formula (I) containing L-α-glutamine or L-α-homoglutamine, wherein X⁸═CR⁵⁷⁰ and R⁵⁷⁰═—C₆H₅(3-F)(4-CF₃) and R⁵⁷⁵═H, namely 7-glutaminyl-(2-(3-fluoro-4-trifluoromethyl-phenyl)-5,6,7,8-tetrahydro(imidazo[1,2-a]pyrazine).

(908) Compound according to general formula (I) containing L-α-glutamine or L-α-homoglutamine, wherein X⁸═CR⁵⁷⁰ and R⁵⁷⁰═-p-C₆H₄F and R⁵⁷⁵═H, namely 7-glutaminyl-(2-(4-fluorophenyl)-5,6,7,8-tetrahydro(imidazo[1,2-a]pyrazine).

(909) Compound according to general formula (I) containing L-α-glutamine or L-α-homoglutamine, wherein X⁸═CR⁵⁷⁰ and R⁵⁷⁰═-p-C₆H₄(OCH₃) and R⁵⁷⁵═H, namely 7-glutaminyl-(2-(4-methoxyphenyl)-5,6,7,8-tetrahydro(imidazo[1,2-a]pyrazine).

(910) Compound according to general formula (I) containing L-α-glutamine or L-α-homoglutamine, wherein X⁸═CR⁵⁷⁰ and R⁵⁷⁰═-p-C₆H₄(OCF₃) and R⁵⁷⁵═H, namely 7-glutaminyl-(2-(4-(trifluoro-methoxy)-phenyl)-5,6,7,8-tetrahydro(imidazo[1,2-a]pyrazine).

(911) Compound according to general formula (I) containing L-α-glutamine or L-α-homoglutamine, wherein X⁸═CR⁵⁷⁰ and R⁵⁷⁰═-p-C₆H₄(OCF₂CF₃) and R⁵⁷⁵═H, namely 1-glutaminyl-(2-(4-(pentafluoroethoxy)-phenyl)-5,6,7,8-tetrahydro(imidazo[1,2-a]pyrazine).

(912) Compound according to general formula (I) containing L-α-glutamine or L-αhomoglutamine, wherein X⁸═CR⁵⁷⁰ and R⁵⁷⁰=-3,4-C₆H₃F₂ and R⁵⁷⁵═H, namely 7-glutaminyl-(2-(3,4-difluoro-phenyl)-5,6,7,8-tetrahydro(imidazo[1,2-a]pyrazine).

(913) Compound according to general formula (I) containing L-α-glutamine or L-α-homoglutamine, wherein X⁸═CR⁵⁷⁰ and R⁵⁷⁰═—CF₂CF₃ and R⁵⁷⁵═H, namely 7-glutaminyl-(2-(pentafluoro-ethyl)-5,6,7,8-tetrahydro(imidazo[1,2-a]pyrazine).

(914) Compound according to general formula (I) containing L-α-glutamine or L-α-homoglutamine, wherein X⁸═CR⁵⁷⁰ and R⁵⁷⁰═H and R⁵⁷⁵═—C≡N, namely 7-glutaminyl-(3-cyano-5,6,7,8-tetrahydro(imidazo[1,2-a]pyrazine).

(915) Compound according to general formula (I) containing L-α-glutamine or L-α-homoglutamine, wherein X⁸═CR⁵⁷⁰ and R⁵⁷⁰═—CH₃ and R⁵⁷⁵═—C≡N, namely 7-glutaminyl-(2-methyl-3-cyano-5,6,7,8-tetrahydro(imidazo[1,2-a]pyrazine).

(916) Compound according to general formula (I) containing L-α-glutamine or L-α-homoglutamine, wherein X⁸═CR⁵⁷⁰ and R⁵⁷⁰═—CF₃ and R⁵⁷⁵═—C≡N, namely 7-glutaminyl-(2-(trifluoromethyl)-3-cyano-5,6,7,8-tetrahydro(imidazo[1,2-a]pyrazine).

(917) Compound according to general formula (I) containing L-α-glutamine or L-α-homoglutamine, wherein X⁸═CR⁵⁷⁰ and R⁵⁷⁰═—CH₂CH₃ and R⁵⁷⁵═—C═N, namely 7-glutaminyl-(2-ethyl-3-cyano-5,6,7,8-tetrahydro(imidazo[1,2-a]pyrazine).

(918) Compound according to general formula (I) containing L-α-glutamine or L-α-homoglutamine, wherein X⁸═CR⁵⁷⁰ and R⁵⁷⁰═—C₆H₅ and R⁵⁷⁵═—C≡N, namely 7-glutaminyl-(2-phenyl-3-cyano-5,6,7,8-tetrahydro(imidazo[1,2-a]pyrazine).

(919) Compound according to general formula (I) containing L-α-glutamine or L-α-homoglutamine, wherein X⁸═CR⁵⁷⁰ and R⁵⁷⁰═-p-C₆H₅(CF₃) and R⁵⁷⁵═—C≡N, namely 7-glutaminyl-(2-(4-trifluoromethyl-phenyl)-3-cyano-5,6,7,8-tetrahydro(imidazo[1,2-a]pyrazine).

(920) Compound according to general formula (I) containing L-α-glutamine or L-α-homoglutamine, wherein X⁸═CR⁵⁷⁰ and R⁵⁷⁰═-p-C₆H₅(CF₂CF₃) and R⁵⁷⁵═—C═N, namely 7-glutaminyl-(2-(4-pentafluoroethyl-phenyl)-3-cyano-5,6,7,8-tetrahydro(imidazo[1,2-a]pyrazine).

(921) Compound according to general formula (I) containing L-α-glutamine or L-α-homoglutamine, wherein X⁸═CR⁵⁷⁰ and R⁵⁷⁰═—C₆H₅(3-F)(4-CF₃) and R⁵⁷⁵═—CEN, namely 7-glutaminyl-(2-(3-fluoro-4-trifluoromethyl-phenyl)-3-cyano-5,6,7,8-tetrahydro(imidazo[1,2-a]pyrazine).

(922) Compound according to general formula (I) containing L-α-glutamine or L-α-homoglutamine, wherein X⁸═CR⁵⁷⁰ and R⁵⁷⁰═-p-C₆H₄F and R⁵⁷⁵═—C═N, namely 7-glutaminyl-(2-(4-fluorophenyl)-3-cyano-5,6,7,8-tetrahydro(imidazo[1,2-a]pyrazine).

(923) Compound according to general formula (I) containing L-α-glutamine or L-α-homoglutamine, wherein X⁸═CR⁵⁷⁰ and R⁵⁷⁰═-p-C₆H₄(OCH₃) and R⁵⁷⁵═—C═N, namely 7-glutaminyl-(2-(4-methoxyphenyl)-3-cyano-5,6,7,8-tetrahydro(imidazo[1,2-a]pyrazine).

(924) Compound according to general formula (I) containing L-α-glutamine or L-α-homoglutamine, wherein X⁸═CR⁵⁷⁰ and R⁵⁷⁰═-p-C₆H₄(OCF₃) and R⁵⁷⁵═—C≡N, namely 7-glutaminyl-(2-(4-(trifluoro-methoxy)-phenyl)-3-cyano-5,6,7,8-tetrahydro(imidazo[1,2-a]pyrazine).

(925) Compound according to general formula (I) containing L-α-glutamine or L-o-homoglutamine, wherein X⁸═CR⁵⁷⁰ and R⁵⁷⁰═-p-C₆H₄(OCF₂CF₃) and R⁵⁷⁵═—C≡N, namely 1-glutaminyl-(2-(4-(pentafluoroethoxy)-3-cyano-phenyl)-5,6,7,8-tetrahydro(imidazo[1,2-a]pyrazine).

(926) Compound according to general formula (I) containing L-α-glutamine or L-α-homoglutamine, wherein X⁸═CR⁵⁷⁰ and R⁵⁷⁰═-3,4-CIH₃F₂ and R⁵⁷⁵═—CmN, namely 7-glutaminyl-(2-(3,4-difluoro-phenyl)-3-cyano-5,6,7,8-tetrahydro(imidazo[1,2-a]pyrazine).

(927) Compound according to general formula (I) containing L-α-glutamine or L-α-homoglutamine, wherein X⁸═CR⁵⁷⁰ and R⁵⁷⁰═—CF₂CF₃ and R⁵⁷⁵═—C═N, namely 7-glutaminyl-(2-(pentafluoro-ethyl)-3-cyano-5,6,7,8-tetrahydro(imidazo[1,2-a]pyrazine).

(928) Compound according to general formula (I) containing L-α-glutamine or L-α-homoglutamine, wherein X⁸═CR⁵⁷⁰ and R⁵⁷⁰═H and R⁵⁷⁵═—COOH, namely 7-glutaminyl-(5,6,7,8-tetrahydro(imidazo[1,2-a]pyrazine-3-carboxylic acid).

(929) Compound according to general formula (I) containing L-α-glutamine or L-α-homoglutamine, wherein X⁸═CR⁵⁷⁰ and R⁵⁷⁰═—CH₃ and R⁵⁷⁵═—COOH, namely 7-glutaminyl-(2-methyl-5,6,7,8-tetrahydro(imidazo[1,2-a]pyrazine-3-carboxylic acid).

(930) Compound according to general formula (I) containing L-α-glutamine or L-α-homoglutamine, wherein X⁸═CR⁵⁷⁰ and R⁵⁷⁰═—CF₃ and R⁵⁷⁵═—COOH, namely 7-glutaminyl-(2-(trifluoromethyl)-5,6,7,8-tetrahydro(imidazo[1,2-a]pyrazine-3-carboxylic acid).

(931) Compound according to general formula (I) containing L-α-glutamine or L-α-homoglutamine, wherein X⁸═CR⁵⁷⁰ and R⁵⁷⁰═—CH₂CH₃ and R⁵⁷⁵═—COOH, namely 7-glutaminyl-(2-ethyl-5,6,7,8-tetrahydro(imidazo[1,2-a]pyrazine-3-carboxylic acid).

(932) Compound according to general formula (I) containing L-α-glutamine or L-α-homoglutamine, wherein X⁸═CR⁵⁷⁰ and R⁵⁷⁰═—C₆H₅ and R⁵⁷⁵═—COOH, namely 7-glutaminyl-(2-phenyl-5,6,7,8-tetrahydro(imidazo[1,2-a]pyrazine-3-carboxylic acid).

(933) Compound according to general formula (I) containing L-α-glutamine or L-α-homoglutamine, wherein X⁸═CR⁵⁷⁰ and R⁵⁷⁰═-p-C₆H₅(CF₃) and R⁵⁷⁵═—COOH, namely 7-glutaminyl-(2-(4-trifluoromethyl-phenyl)-5,6,7,8-tetrahydro(imidazo[1,2-a]pyrazine-3-carboxylic acid).

(934) Compound according to general formula (I) containing L-α-glutamine or L-α-homoglutamine, wherein X⁸═CR⁵⁷⁰ and R⁵⁷⁰═-p-C₆H₅(CF₂CF₃) and R⁵⁷⁵═—COOH, namely 7-glutaminyl-(2-(4-pentafluoroethyl-phenyl)-5,6,7,8-tetrahydro(imidazo[1,2-a]pyrazine-3-carboxylic acid).

(935) Compound according to general formula (I) containing L-α-glutamine or L-α-homoglutamine, wherein X⁸═CR⁵⁷⁰ and R⁵⁷⁰═—C₆H₅(3-F)(4-CF₃) and R⁵⁷⁵═—COOH, namely 7-glutaminyl-(2-(3-fluoro-4-trifluoromethyl-phenyl)-5,6,7,8-tetrahydro(imidazo[1,2-a]pyrazine-3-carboxylic acid).

(936) Compound according to general formula (I) containing L-α-glutamine or L-α-homoglutamine, wherein X⁸═CR⁵⁷⁰ and R⁵⁷⁰═-p-C₆H₄F and R⁵⁷⁵═—COOH, namely 7-glutaminyl-(2-(4-fluorophenyl)-5,6,7,8-tetrahydro(imidazo[1,2-a]pyrazine-3-carboxylic acid).

(937) Compound according to general formula (I) containing L-α-glutamine or L-α-homoglutamine, wherein X⁸═CR⁵⁷⁰ and R⁵⁷⁰═-p-C₆H₄(OCH₃) and R⁵⁷⁵═—COOH, namely 7-glutaminyl-(2-(4-methoxyphenyl)-5,6,7,8-tetrahydro(imidazo[1,2-a]pyrazine-3-carboxylic acid).

(938) Compound according to general formula (I) containing L-α-glutamine or L-α-homoglutamine, wherein X⁸═CR⁵⁷⁰ and R⁵⁷⁰═-p-C₆H₄(OCF₃) and R⁵⁷⁵═—COOH, namely 7-glutaminyl-(2-(4-(trifluoro-methoxy)-phenyl)-5,6,7,8-tetrahydro(imidazo[1,2-a]pyrazine-3-carboxylic acid).

(939) Compound according to general formula (I) containing L-α-glutamine or L-α-homoglutamine, wherein X⁸═CR⁵⁷⁰ and R⁵⁷⁰═-p-C₆H₄(OCF₂CF₃) and R⁵⁷⁵═—COOH, namely 1-glutaminyl-(2-(4-(pentafluoroethoxy)-phenyl)-5,6,7,8-tetrahydro(imidazo[1,2-a]pyrazine-3-carboxylic acid).

(940) Compound according to general formula (I) containing L-α-glutamine or L-α-homoglutamine, wherein X⁸═CR⁵⁷⁰ and R⁵⁷⁰═-3,4-C₆H₃F₂ and R⁵⁷⁵═—COOH, namely 7-glutaminyl-(2-(3,4-difluoro-phenyl)-5,6,7,8-tetrahydro(imidazo[1,2-a]pyrazine-3-carboxylic acid).

(941) Compound according to general formula (I) containing L-α-glutamine or L-α-homoglutamine, wherein X⁸═CR⁵⁷⁰ and R⁵⁷⁰═—CF₂CF₃ and R⁵⁷⁵═—COOH, namely 7-glutaminyl-(2-(pentafluoro-ethyl)-5,6,7,8-tetrahydro(imidazo[1,2-a]pyrazine-3-carboxylic acid).

(942) Compound according to general formula (I) containing L-α-glutamine or L-α-homoglutamine, wherein X⁸═CR⁵⁷⁰ and R⁵⁷⁰═H and R⁵⁷⁵═—B(OH)₂, namely 7-glutaminyl-(5,6,7,8-tetrahydro(imidazo[1,2-a]pyrazine-3-boronic acid).

(943) Compound according to general formula (I) containing L-(X-glutamine or L-α-homoglutamine, wherein X⁸═CR⁵⁷⁰ and R⁵⁷⁰═—CH₃ and R⁵⁷⁵═—B(OH)₂, namely 7-glutaminyl-(2-methyl-5,6,7,8-tetrahydro(imidazo[1,2-a]pyrazine-3-boronic acid).

(944) Compound according to general formula (I) containing L-α-glutamine or L-α-homoglutamine, wherein X⁸═CR⁵⁷⁰ and R⁵⁷⁰═—CF₃ and R⁵⁷⁵═—B(OH)₂, namely 7-glutaminyl-(2-(trifluoromethyl)-5,6,7,8-tetrahydro(imidazo[1,2-a]pyrazine-3-boronic acid).

(945) Compound according to general formula (I) containing L-α-glutamine or L-α-homoglutamine, wherein X⁸═CR⁵⁷⁰ and R⁵⁷⁰═—CH₂CH₃ and R⁵⁷⁵═—B(OH)₂, namely 7-glutaminyl-(2-ethyl-5,6,7,8-tetrahydro(imidazo[1,2-a]pyrazine-3-boronic acid).

(946) Compound according to general formula (I) containing L-α-glutamine or L-α-homoglutamine, wherein X⁸═CR⁵⁷⁰ and R⁵⁷⁰═—C₆H₅ and R⁵⁷⁵═—B(OH)₂, namely 7-glutaminyl-(2-phenyl-5,6,7,8-tetrahydro(imidazo[1,2-a]pyrazine-3-boronic acid).

(947) Compound according to general formula (I) containing L-α-glutamine or L-α-homoglutamine, wherein X⁸═CR⁵⁷⁰ and R⁵⁷⁰═p-C₆H₅(CF₃) and R⁵⁷⁵═—B(OH)₂, namely 7-glutaminyl-(2-(4-trifluoromethyl-phenyl)-5,6,7,8-tetrahydro(imidazo[1,2-a]pyrazine-3-boronic acid).

(948) Compound according to general formula (I) containing L-α-glutamine or L-α-homoglutamine, wherein X⁸═CR⁵⁷⁰ and R⁵⁷⁰═-p-C₆H₅(CF₂CF₃) and R⁵⁷⁵═—B(OH)₂, namely 7-glutaminyl-(2-(4-pentafluoroethyl-phenyl)-5,6,7,8-tetrahydro(imidazo[1,2-a]pyrazine-3-boronic acid).

(949) Compound according to general formula (I) containing L-α-glutamine or L-α-homoglutarine, wherein X⁸═CR⁵⁷⁰ and R⁵⁷⁰═—C₆H₅(3-F)(4-CF₃) and R⁵⁷⁵═—B(OH)₂, namely 7-glutaminyl-(2-(3-fluoro-4-trifluoromethyl-phenyl)-5,6,7,8-tetrahydro(imidazo[1,2-a]pyrazine-3-boronic acid).

(950) Compound according to general formula (I) containing L-α-glutamine or L-α-homoglutamine, wherein X⁸═CR⁵⁷⁰ and R⁵⁷⁰═-p-C₆H₄F and R⁵⁷⁵═—B(OH)₂, namely 7-glutaminyl-(2-(4-fluorophenyl)-5,6,7,8-tetrahydro(imidazo[1,2-a]pyrazine-3-boronic acid).

(951) Compound according to general formula (I) containing L-α-glutamine or L-α-homoglutamine, wherein X⁸═CR⁵⁷⁰ and R⁵⁷⁰═-p-C₆H₄(OCH₃) and R⁵⁷⁵═—B(OH)₂, namely 7-glutaminyl-(2-(4-methoxyphenyl)-5,6,7,8-tetrahydro(imidazo[1,2-a]pyrazine-3-boronic acid).

(952) Compound according to general formula (I) containing L-α-glutamine or L-α-homoglutamine, wherein X⁸═CR⁵⁷⁰ and R⁵⁷⁰═-p-C₆H₄(OCF₃) and R⁵⁷⁵═—B(OH)₂, namely 7-glutaminyl-(2-(4-(trifluoro-methoxy)-phenyl)-5,6,7,8-tetrahydro(imidazo[1,2-a]pyrazine-3-boronic acid).

(953) Compound according to general formula (I) containing L-α-glutamine or L-α-homoglutamine, wherein X⁸═CR⁵⁷⁰ and R⁵⁷⁰═-p-C₆H₄(OCF₂CF₃) and R⁵⁷⁵═—B(OH)₂, namely 1-glutaminyl-(2-(4-(pentafluoroethoxy)-phenyl)-5,6,7,8-tetrahydro(imidazo[1,2-a]pyrazine-3-boronic acid).

(954) Compound according to general formula (I) containing L-α-glutamine or L-αhomoglutamine, wherein X⁸═CR⁵⁷⁰ and R⁵⁷⁰═-3,4-C₆H₃F₂ and R⁵⁷⁵═—B(OH)₂, namely 7-glutaminyl-(2-(3,4-difluoro-phenyl)-5,6,7,8-tetrahydro(imidazo[1,2-a]pyrazine-3-boronic acid).

(955) Compound according to general formula (I) containing L-α-glutamine or L-α-homoglutamine, wherein X⁸═CR⁵⁷⁰ and R⁵⁷⁰═—CF₂CF₃ and R⁵⁷⁵═—B(OH)₂, namely 7-glutaminyl-(2-(pentafluoro-ethyl)-5,6,7,8-tetrahydro(imidazo[1,2-a]pyrazine-3-boronic acid).

(956) Compound according to general formula (I) containing L-α-glutamine or L-α-homoglutamine, wherein X⁸═CR⁵⁷⁰ and R⁵⁷⁰═H and R⁵⁷⁵═—P(═O) (OR⁵⁹⁶)(597) and R⁵=—C₆H₅ and R⁵⁹⁷═—C₆H₅, namely 7-glutaminyl-(5,6,7,8-tetrahydro(imidazo[1,2-a]pyrazine-3-phosphonic acid diphenyl ester).

(957) Compound according to general formula (I) containing L-α-glutamine or L-α-homoglutamine, wherein X⁸═CR⁵⁷⁰ and R⁵⁷⁰═—CH₃ and R⁵⁷⁵═—P(═O) (OR⁵⁹⁶)(OR⁵⁹⁷) and R⁵⁹⁶═—C₆H₅ and R⁵⁹⁷═—C₆H₅, namely 7-glutaminyl-(2-methyl-5,6,7,8-tetrahydro(imidazo[1,2-a]pyrazine-3-phosphonic acid diphenyl ester).

(958) Compound according to general formula (I) containing L-α-glutamine or L-α-homoglutamine, wherein X⁸═CR⁵⁷⁰ and R⁵⁷⁰═—CF₃ and R⁵⁷═P(═O) (OR⁵⁹⁶)(OR⁵⁹⁷) and R⁵⁹⁶═—C₆H₅ and R⁵⁹⁷═—C₆H₅, namely 7-glutaminyl-(2-(trifluoromethyl)-5,6,7,8-tetrahydro(imidazo[1,2-a]pyrazine-3-phosphonic acid diphenyl ester).

(959) Compound according to general formula (I) containing L-α-glutamine or L-α-homoglutamine, wherein X⁸═CR⁵⁷⁰ and R⁵⁷⁰═—CH₂CH₃ and R⁵⁷⁵═—P(═O) (OR⁵⁹⁶)(OR⁵⁹⁷) and R⁵═—C₆H₅ and R⁵⁹⁷═—C₆H₅, namely 7-glutaminyl-(2-ethyl-5,6,7,8-tetrahydro(imidazo[1,2-a]pyrazine-3-phosphonic acid diphenyl ester).

(960) Compound according to general formula (I) containing L-α-glutamine or L-α-homoglutamine, wherein X⁸═CR⁵⁷⁰ and R⁵⁷⁰═—C₆H₅ and R⁵⁷⁵═—P(═O) (OR⁵⁹⁶)(OR⁵⁹⁷) and R⁵⁹⁶═—C₆H₅ and R⁵⁹⁷═—C₆H₅, namely 7-glutaminyl-(2-phenyl-5,6,7,8-tetrahydro(imidazo[1,2-a]pyrazine-3-phosphonic acid diphenyl ester).

(961) Compound according to general formula (I) containing L-α-glutamine or L-α-homoglutamine, wherein X⁸═CR⁵⁷⁰ and R⁵⁷⁰═-p-C₆H₅(CF₃) and R⁵⁷⁵═—P(═O) (OR⁵⁹⁶)(OR⁵⁹⁷) and R⁵⁹⁶═—C₆H₅ and R⁵⁹⁷═—C₆H₅, namely 7-glutaminyl-(2-(4-trifluoromethyl-phenyl)-5,6,7,8-tetrahydro(imidazo[1,2-a]pyrazine-3-phosphonic acid diphenyl ester).

(962) Compound according to general formula (I) containing L-α-glutamine or L-α-homoglutamine, wherein X⁸═CR⁵⁷⁰ and R⁵⁷⁰═-p-C₆Hs(CF₂CF₃) and R⁵⁷⁵═_P(═O) (OR⁵⁹⁶)(OR⁵⁹⁷) and R⁵⁹⁶═—C₆H₅ and R⁵⁹⁷═—C₆H₅, namely 7-glutaminyl-(2-(4-pentafluoroethyl-phenyl)-5,6,7,8-tetrahydro(imidazo[1,2-a]pyrazine-3-phosphonic acid diphenyl ester).

(963) Compound according to general formula (I) containing L-α-glutamine or L-α-homoglutamine, wherein X⁸═CR⁵⁷⁰ and R⁵⁷⁰═—C₆H₅(3-F)(4-CF₃) and R⁵⁷⁵═—P(═O) (OR⁵⁹⁶)(OR⁵⁹⁷) and R⁵⁹⁶═—C₆H⁵ and R⁵⁹⁷═—C₆H₅, namely 7-glutaminyl-(2-(3-fluoro-4-trifluoromethyl-phenyl)-5,6,7,8-tetrahydro(imidazo[1,2-a]pyrazine-3-phosphonic acid diphenyl ester).

(964) Compound according to general formula (I) containing L-α-glutamine or L-α-homoglutamine, wherein X⁸═CR⁵⁷⁰ and R⁵⁷⁰═-p-C₆H₄F and R⁵⁷-=—P(═O) (OR⁵⁹⁶)(OR⁵⁹⁷) and R⁵⁹⁶═—C₆H, and R⁵⁹⁷═—C₆H₅, namely 7-glutaminyl-(2-(4-fluorophenyl)-5,6,7,8-tetrahydro(imidazo[1,2-a]pyrazine-3-phosphonic acid diphenyl ester).

(965) Compound according to general formula (I) containing L-α-glutamine or L-α-homoglutamine, wherein X⁸═CR⁵⁷⁰ and R⁵⁷⁰═-p-C₆H₄(OCH₃) and R⁵⁷⁵═_P(═O) (OR⁵⁹⁶)(OR⁵⁹⁷) and R⁵⁹═—C₆H₅ and R⁵⁹⁷═—C₆H₅, namely 7-glutaminyl-(2-(4-methoxyphenyl)-5,6,7,8-tetrahydro(imidazo[1,2-a]pyrazine-3-phosphonic acid diphenyl ester).

(966) Compound according to general formula (I) containing L-α-glutamine or L-α-homoglutamine, wherein X⁸═CR⁵⁷⁰ and R⁵⁷⁰═-p-C₆H₅(OCF₃) and R⁵⁷⁵═—P(═O) (OR⁵⁹⁶)(OR⁵⁹⁷) and R⁵⁹⁶═—C₆H₅ and R⁵⁹⁷═—C₆H₅, namely 7-glutaminyl-(2-(4-(trifluoro-methoxy)-phenyl)-5,6,7,8-tetrahydro(imidazo[1,2-a]pyrazine-3-phosphonic acid diphenyl ester).

(967) Compound according to general formula (I) containing L-α-glutamine or L-α-homoglutamine, wherein X⁸═CR⁵⁷⁰ and R⁵⁷⁰═-p-C₆H₄(OCF₂CF₃) and R⁵⁷⁵═—P(═O) (OR⁵⁹⁶)(OR⁵⁹⁷) and R⁵⁹⁶═—C₆H₅ and R⁵⁹⁷═—C₆H₅, namely 1-glutaminyl-(2-(4-(pentafluoroethoxy)-phenyl)-5,6,7,8-tetrahydro(imidazo[1,2-a]pyrazine-3-phosphonic acid diphenyl ester).

(968) Compound according to general formula (I) containing L-α-glutamine or L-α-homoglutamine, wherein X⁸═CR⁵⁷⁰ and R⁵⁷⁰═-3,4-C₆H₃F₂ and R⁵⁷⁵═—P(═O) (OR⁵⁹⁶)(OR⁵⁹⁷) and R⁵⁹⁶═—C₆H₅ and R⁵⁹⁷═—C₆H₅, namely 7-glutaminyl-(2-(3,4-difluoro-phenyl)-5,6,7,8-tetrahydro(imidazo[1,2-a]pyrazine-3-phosphonic acid diphenyl ester).

(969) Compound according to general formula (I) containing L-α-glutamine or L-α-homoglutamine, wherein X⁸═CR⁵⁷⁰ and R⁵⁷⁰═—CF₂CF₃ and R⁵⁷⁵═_P(═O) (OR⁵⁹⁶)(OR⁵⁹⁷) and R⁵⁹⁶═—C₆H₅ and R⁵⁹⁷═—C₆H₅, namely 7-glutaminyl-(2-(pentafluoro-ethyl)-5,6,7,8-tetrahydro(imidazo[1,2-a]pyrazine-3-phosphonic acid diphenyl ester).

(970) Compound according to general formula (I) containing L-α-glutamine or L-α-homoglutamine, wherein X⁸═CR⁵⁷⁰ and R⁵⁷⁰═—H and R⁵⁷⁵═—C₆H₅, namely 7-glutaminyl-(3-phenyl-5,6,7,8-tetrahydro(imidazo[1,2-a]pyrazine).

(971) Compound according to general formula (I) containing L-α-glutamine or L-α-homoglutamine, wherein X⁸═CR⁵⁷⁰ and R⁵⁷⁰═—H and R⁵⁷⁵═—CH₂C₆H₅, namely 7-glutaminyl-(3-benzyl-5,6,7,8-tetrahydro(imidazo[1,2-a]pyrazine).

(972) Compound according to general formula (I) containing L-α-glutamine or L-α-homoglutamine, wherein X⁸═CR⁵⁷⁰ and R⁵⁷⁰═—H and R⁵⁷⁵=2H-tetrazol-5-yl, namely 7-glutaminyl-(3-(2H-tetrazol-5-yl)-5,6,7,8-tetrahydro(imidazo[1,2-a]pyrazine).

(973) Compound according to general formula (I) containing L-α-glutamine or L-α-homoglutamine, wherein x⁸═N and R⁵⁷⁵═—H, namely 7-glutaminyl-(5,6,7,8-tetrahydro-1,2,4-triazolo[4,3-a]pyrazine).

(974) Compound according to general formula (I) containing L-α-glutamine or L-α-homoglutamine, wherein X⁸═N and R⁵⁷⁵═—CH₃, namely 7-glutaminyl-(3-methyl-5,6,7,8-tetrahydro-1,2,4-triazolo[4,3-a]pyrazine).

(975) Compound according to general formula (I) containing L-(X-glutamine or L-α-homoglutamine, wherein X⁸═N and R⁵⁷⁵═—CH₂CH₃, namely 7-glutaminyl-(3-ethyl-5,6,7,8-tetrahydro-1,2,4-triazolo[4,3-a]pyrazine).

(976) Compound according to general formula (I) containing L-α-glutamine or L-α-homoglutamine, wherein X⁸═N and R⁵⁷⁵═—CF₃, namely 7-glutaminyl-(3-trifluoro-methyl-5,6,7,8-tetrahydro-1,2,4-triazolo [4,3-a]pyrazine).

(977) Compound according to general formula (I) containing L-α-glutamine or L-α-homoglutamine, wherein X⁸═N and R⁵⁷⁵═—CF₂CF₃, namely 7-glutaminyl-(3-pentafluoroethyl-5,6,7,8-tetrahydro-1,2,4-triazolo[4,3-a]pyrazine).

(978) Compound according to general formula (I) containing L-(X-glutamine or L-α-homoglutamine, wherein X⁸=N and R⁵⁷⁵═—C₆H₅, namely 7-glutaminyl-(3-phenyl-5,6,7,8-tetrahydro-1,2,4-triazolo[4,3-a]pyrazine).

(979) Compound according to general formula (I) containing L-α-glutamine or L-α-homoglutamine, wherein X⁸═N and R⁵⁷⁵═—C₆H₄(4-F), namely 7-glutaminyl-(3-(4-fluoro-phenyl)-5,6,7,8-tetrahydro-1,2,4-triazolo[4,3-a]pyrazine).

(980) Compound according to general formula (I) containing L-α-glutamine or L-α-homoglutamine, wherein X⁸═N and R⁵⁷¹═—C₆H₄—CF₃), namely 7-glutaminyl-(3-(4-trifluoromethyl-phenyl)-5,6,7,8-tetrahydro-1,2,4-triazolo[4,3-a]pyrazine).

(981) Compound according to general formula (I) containing L-α-glutamine or L-α-homoglutamine, wherein X⁸═N and R⁵⁷⁵═—C₆H₃(3-F)(4-CF₃), namely 7-glutaminyl-(3-(3-fluoro-4-trifluoromethyl-phenyl)-5,6,7,8-tetrahydro-1,2,4-triazolo[4,3-a]pyrazine).

(982) Compound according to general formula (I) containing L-α-glutamine or L-α-homoglutamine, wherein X⁸=N and R⁵⁷⁵═—CH₂CF₃, namely 7-glutaminyl-(3-(2,2,2-trifluoro-eth-1-yl)-5,6,7,8-tetrahydro-1,2,4-triazolo[4,3-a]pyrazine).

(983) Compound according to general formula (I) containing L-α-glutamine or L-α-homoglutamine, wherein X⁸=N and R⁵⁷⁵═—C≡N, namely 7-glutaminyl-(3-cyano-5,6,7,8-tetrahydro-1,2,4-triazolo[4,3-a]pyrazine).

(984) Compound according to general formula (I) containing L-α-glutamine or L-α-homoglutamine, wherein X⁸═N and R⁵⁷⁵═—COOH, namely 7-glutaminyl-(5,6,7,8-tetrahydro-1,2,4-triazolo[4,3-a]pyrazine-3-carboxylic acid).

(985) Compound according to general formula (I) containing L-α-glutamine or L-α-homoglutamine, wherein X⁸═N and R⁵⁷⁵═—B(OH)₂, namely 7-glutaminyl-(5,6,7,8-tetrahydro-1,2,4-triazolo[4,3-a]pyrazine-3-boronic acid).

(986) Compound according to general formula (I) containing L-α-glutamine or L-α--homoglutamine, wherein X⁸═N and R⁵⁷⁵═—P(═O) (OR⁵⁹⁶)(OR⁵⁹⁷) and R⁵⁹⁶═—C₆H₅ and R⁵⁹⁷=—C₆H₅, namely 7-glutaminyl-(5,6,7,8-tetrahydro-1,2,4-triazolo[4,3-a]pyrazine-3-phosphonic acid diphenyl ester).

Examples for Prolin Mimetics of Formula (IXa):

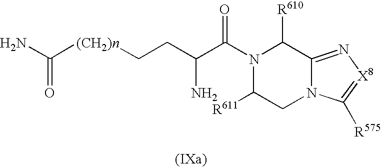

| Ex. | n | R⁶¹⁰ | R⁶¹¹ | X⁸ | R⁵⁷⁵ |
|---|---|---|---|---|---|
| 1000 | 0 | 4-F—C₆H₄ | H | C—CF₃ | H |
| 1001 | 1 | 4-F—C₆H₄ | H | C—CF₃ | H |
| 1002 | 0 | 3-(CF₃)C₆H₄ | H | C—CF₃ | H |
| 1003 | 1 | 3-(CF₃)C₆H₄ | H | C—CF₃ | H |
| 1005 | 0 | Me | H | C—CF₃ | H |
| 1006 | 1 | Me | H | C—CF₃ | H |
| 1007 | 0 | Et | H | C—CF₃ | H |
| 1008 | 1 | Et | H | C—CF₃ | H |
| 1009 | 0 | Isopropyl | H | C—CF₃ | H |
| 1010 | 1 | Isopropyl | H | C—CF₃ | H |
| 1011 | 0 | H | 4-F—C₆H₄ | C—CF₃ | H |
| 1012 | 1 | H | 4-F—C₆H₄ | C—CF₃ | H |
| 1013 | 0 | H | Me | C—CF₃ | H |
| 1014 | 1 | H | Me | C—CF₃ | H |
| 1015 | 0 | H | 3-(CF₃)C₆H₄ | C—CF₃ | H |
| 1016 | 1 | H | 3-(CF₃)C₆H₄ | C—CF₃ | H |
| 1017 | 0 | Et | H | N | CF₃ |
| 1018 | 1 | Et | H | N | CF₃ |
| 1019 | 0 | Me | H | N | CF₃ |
| 1020 | 1 | Me | H | N | CF₃ |
| 1021 | 0 | H | Et-C₆H₄ | CH | Me |
| 1022 | 1 | H | Et-C₆H₄ | CH | Me |
| 1023 | 0 | H | Et-C₆H₄ | N | Me |
| 1024 | 1 | H | Et-C₆H₄ | N | Me |
| 1025 | 0 | Me | H | N | Me |
| 1026 | 1 | Me | H | N | Me |
| 1027 | 0 | Me | H | C—CF₃ | H |
| 1028 | 1 | Me | H | C—CF₃ | H |
| 1029 | 0 | 4-F—C₆H₄ | H | C—CF₃ | H |
| 1030 | 1 | 4-F—C₆H₄ | H | C—CF₃ | H |
| 1031 | 0 | CO₂Me | H | C—CF₃ | H |
| 1032 | 1 | CO₂Me | H | C—CF₃ | H |
| 1033 | 0 | H | Me | N | Me |
| 1034 | 1 | H | Me | N | Me |
| 1035 | 0 | Me | Me | N | Me |
| 1036 | 1 | Me | Me | N | Me |

Examples for Prolin Mimetics of Formula (XIII):

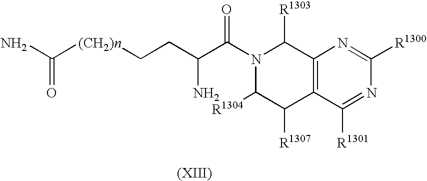

| Ex. | n | R¹³⁰⁰ | R¹³⁰¹ | R¹³⁰³ | R¹³⁰⁴ | R¹³⁰⁷ |
|---|---|---|---|---|---|---|
| 1200 | 0 | CF₃ | H | H | H | H |
| 1201 | 1 | CF₃ | H | H | H | H |
| 1203 | 0 | CF₃ | OCHMe₂ | H | H | H |
| 1204 | 1 | CF₃ | OCHMe₂ | H | H | H |
| 1205 | 0 | CF₃ | NHMe | H | H | H |
| 1206 | 1 | CF₃ | NHMe | H | H | H |
| 1207 | 0 | 4-(CF₃)C₆H₄ | OH | H | H | H |
| 1208 | 1 | 4-(CF₃)C₆H₄ | OH | H | H | H |
| 1209 | 0 | 4-F-C₆H₄ | H | H | H | H |
| 1210 | 1 | 4-F-C₆H₄ | H | H | H | H |
| 1211 | 0 | H | H | H | H | H |
| 1212 | 1 | H | H | H | H | H |
| 1213 | 0 | 3-Pyridyl | H | H | H | H |
| 1214 | 1 | 3-Pyridyl | H | H | H | H |
| 1215 | 0 | Me | H | H | H | H |
| 1216 | 1 | Me | H | H | H | H |
| 1217 | 0 | 3-F-C₆H₄ | H | H | H | H |
| 1218 | 1 | 3-F-C₆H₄ | H | H | H | H |
| 1219 | 0 | Ph | H | H | H | H |
| 1220 | 1 | Ph | H | H | H | H |
| 1221 | 0 | NMe₂ | H | H | H | H |
| 1222 | 1 | NMe₂ | H | H | H | H |
| 1223 | 0 | 4-morpholino | H | H | H | H |
| 1224 | 1 | 4-morpholino | H | H | H | H |
| 1225 | 0 | 4-(OCF₃)C₆H₄ | H | H | H | H |
| 1226 | 1 | 4-(OCF₃)C₆H₄ | H | H | H | H |
| 1227 | 0 | Cyclopropyl | H | H | H | H |
| 1228 | 1 | Cyclopropyl | H | H | H | H |
| 1229 | 0 | 4-(NMe₂)C₆H₄ | H | H | H | H |
| 1230 | 1 | 4-(NMe₂)C₆H₄ | H | H | H | H |
| 1231 | 0 | 4-pyridyl | H | H | H | H |
| 1232 | 1 | 4-pyridyl | H | H | H | H |
| 1233 | 0 | 4-(SO₂Me)C₆H₄ | H | H | H | H |
| 1234 | 1 | 4-(SO₂Me)C₆H₄ | H | H | H | H |
| 1235 | 0 | 3-Me-4-NO₂- | H | H | H | H |
| | | imidazol-2-yl | | | | |
| 1236 | 1 | 3-Me-4-NO₂- | H | H | H | H |
| | | imidazol-2-yl | | | | |
| 1237 | 0 | 4-(SO₂CF₃)C₆H₄ | H | H | H | H |
| 1238 | 1 | 4-(SO₂CF₃)C₆H₄ | H | H | H | H |
| 1239 | 0 | 4-(SO₂NH₂)C₆H₄ | H | H | H | H |
| 1240 | 1 | 4-(SO₂NH₂)C₆H₄ | H | H | H | H |
| 1241 | 0 | 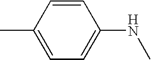 | H | H | H | H |
| 1242 | 1 | 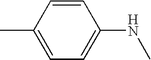 | H | H | H | H |
| 1243 | 0 | 2-pyrazinyl | H | H | H | H |
| 1244 | 1 | 2-pyrazinyl | H | H | H | H |
| 1245 | 0 | CF₃ | H | Me | H | H |
| 1246 | 1 | CF₃ | H | Me | H | H |
| 1247 | 0 | 4-Me-C₆H₄ | H | H | H | H |
| 1248 | 1 | 4-Me-C₆H₄ | H | H | H | H |
| 1249 | 0 | 3,4-(Cl)₂C₆H₄ | H | H | H | H |
| 1250 | 1 | 3,4-(Cl)₂C₆H₄ | H | H | H | H |
| 1251 | 0 | 4-Cl—C₆H₄ | H | H | H | H |
| 1252 | 1 | 4-Cl—C₆H₄ | H | H | H | H |
| 1253 | 0 | 2-Cl—C₆H₄ | H | H | H | H |
| 1254 | 1 | 2-Cl—C₆H₄ | H | H | H | H |
| 1255 | 0 | 2-F—C₆H₄ | H | H | H | H |
| 1256 | 1 | 2-F—C₆H₄ | H | H | H | H |
| 1257 | 0 | 2-pyridyl | H | H | H | H |
| 1258 | 1 | 2-pyridyl | H | H | H | H |
| 1259 | 0 | 4-(CONH₂)C₆H₄ | H | H | H | H |
| 1260 | 1 | 4-(CONH₂)C₆H₄ | H | H | H | H |
| 1261 | 0 | 2-pyrazinyl | CF₃ | H | H | H |
| 1262 | 1 | 2-pyrazinyl | CF₃ | H | H | H |
| 1263 | 0 | 4-(NH₂)C₆H₄ | H | H | H | H |
| 1264 | 1 | 4-(NH₂)C₆H₄ | H | H | H | H |
| 1265 | 0 | H | CF₃ | H | H | H |
| 1266 | 1 | H | CF₃ | H | H | H |
| 1267 | 0 | 4-(SO₂Me)C₆H₄ | CF₃ | H | H | H |
| 1268 | 1 | 4-(SO₂Me)C₆H₄ | CF₃ | H | H | H |
| 1269 | 0 | 4-(NHSO₂Me)C₆H₄ | H | H | H | H |
| 1270 | 1 | 4-(NHSO₂Me)C₆H₄ | H | H | H | H |

### Examples for Prolin Mimetics of Formula (XIV)

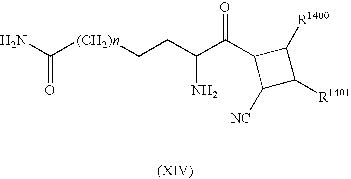

| Ex. | n | R¹⁴⁰⁰ | R¹⁴⁰¹ |
|---|---|---|---|
| 1300 | 0 | H | H |
| 1301 | 1 | H | H |
| 1302 | 0 | H | F |
| 1303 | 1 | H | F |

Examples for Prolin Mimetics of Formula (XV):

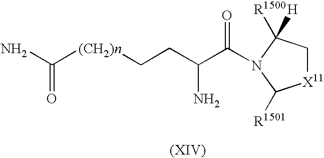

| Ex. | n | R¹⁵⁰⁰ | R¹⁵⁰¹ | X¹¹ |
|---|---|---|---|---|
| 1400 | 0 | CN | Me | CH₂ |
| 1401 | 1 | CN | Me | CH₂ |
| 1402 | 0 | CN | Me | CHF |
| 1403 | 1 | CN | Me | CHF |
| 1404 | 0 | CN | Me | CF₂ |
| 1405 | 1 | CN | Me | CF₂ |
| 1406 | 0 | CN | Et | CH₂ |
| 1407 | 1 | CN | Et | CH₂ |
| 1408 | 0 | CN | Et | CHF |
| 1409 | 1 | CN | Et | CHF |
| 1410 | 0 | CN | Et | CF₂ |
| 1411 | 1 | CN | Et | CF₂ |
| 1412 | 0 | CN | Ethynyl | CH₂ |
| 1413 | 1 | CN | Ethynyl | CH₂ |
| 1414 | 0 | CN | Ethynyl | CHF |
| 1415 | 1 | CN | Ethynyl | CHF |
| 1416 | 0 | CN | Ethynyl | CF₂ |
| 1417 | 1 | CN | Ethynyl | CF₂ |
| 1418 | 0 | CN | Vinyl | CH₂ |
| 1419 | 1 | CN | Vinyl | CH₂ |
| 1420 | 0 | CN | Vinyl | CHF |
| 1421 | 1 | CN | Vinyl | CHF |
| 1422 | 0 | CN | Vinyl | CF₂ |
| 1423 | 1 | CN | Vinyl | CF₂ |
| 1424 | 0 | CN | Prop-1-ynyl | CH₂ |
| 1425 | 1 | CN | Prop-1-ynyl | CH₂ |
| 1426 | 0 | CN | Prop-1-ynyl | CHF |
| 1427 | 1 | CN | Prop-1-ynyl | CHF |
| 1428 | 0 | CN | Prop-1-ynyl | CF₂ |
| 1429 | 1 | CN | Prop-1-ynyl | CF₂ |

Examples for Prolin Mimetics of Formula (X):

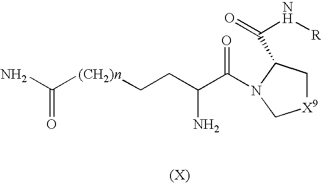

| Ex. | n | X⁹ | R |
|---|---|---|---|
| 1500 | 0 | S | 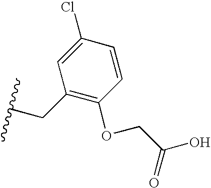 |
| 1501 | 1 | S | 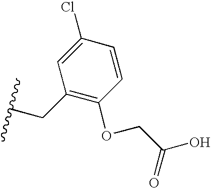 |
| 1502 | 0 | CH₂ | 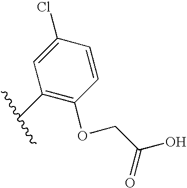 |
| 1503 | 1 | CH₂ | 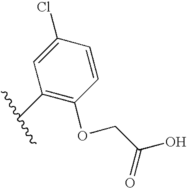 |
| 1504 | 0 | S | 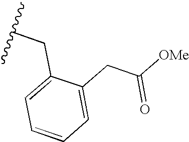 |
| 1505 | 1 | S | 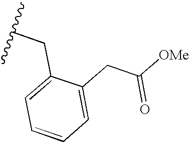 |
| 1506 | 0 | CH₂ | 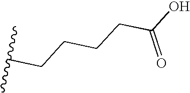 |
| 1507 | 1 | CH₂ | 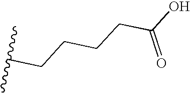 |
| 1508 | 0 | S | 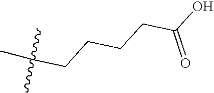 |
| 1509 | 1 | S | 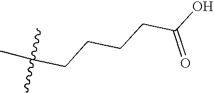 |
| 1510 | 0 | CH₂ | 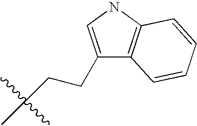 |
| 1511 | 1 | CH₂ | 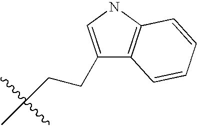 |
| 1512 | 0 | CH₂ | 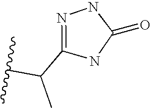 |
| 1513 | 1 | CH₂ | 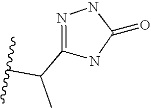 |
| 1514 | 0 | CH₂ | 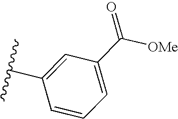 |
| 1515 | 1 | CH₂ | 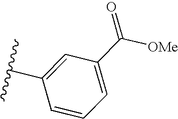 |
| 1516 | 0 | CH₂ | 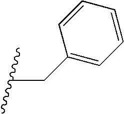 |
| 1517 | 1 | CH₂ | 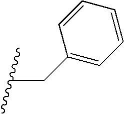 |
| 1518 | 0 | CH₂ | 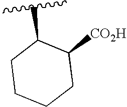 |
| 1519 | 1 | CH₂ | 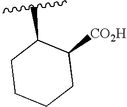 |
| 1520 | 0 | CH₂ | 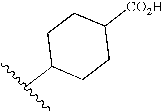 |
| 1521 | 1 | CH₂ | 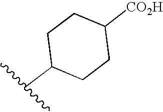 |
| 1522 | 0 | CH₂ | 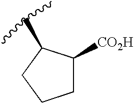 |
| 1523 | 1 | CH₂ | 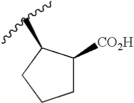 |
| 1524 | 0 | CH₂ | 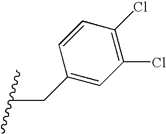 |
| 1525 | 1 | CH₂ | 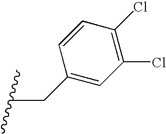 |
| 1526 | 0 | CH₂ | 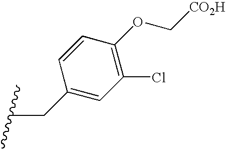 |
| 1527 | 1 | CH₂ | 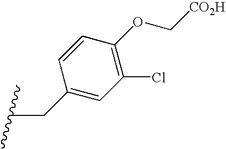 |
| 1528 | 0 | CH₂ | 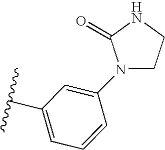 |
| 1529 | 1 | CH₂ | 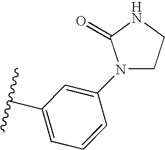 |
| 1530 | 0 | CH₂ | 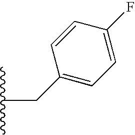 |
| 1531 | 1 | CH₂ | 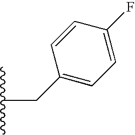 |
| 1532 | 0 | CH₂ | 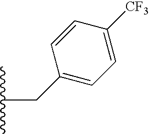 |
| 1533 | 1 | CH₂ | 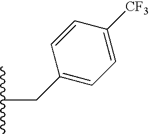 |
| 1534 | 0 | CH₂ | 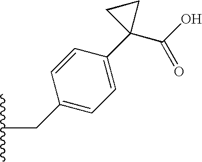 |
| 1535 | 1 | CH₂ | 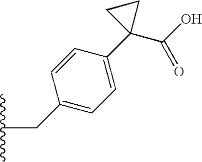 |
| 1536 | 0 | CH₂ | 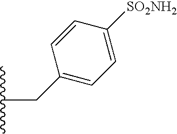 |
| 1537 | 1 | CH₂ | 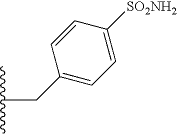 |
| 1538 | 0 | CH₂ | 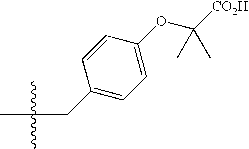 |
| 1539 | 1 | CH₂ | 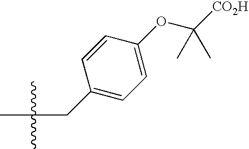 |
| 1540 | 0 | S | 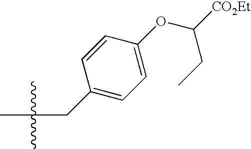 |
| 1541 | 1 | S | 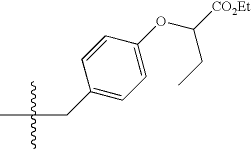 |
| 1542 | 0 | CH₂ | 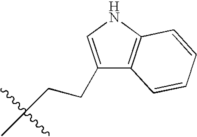 |
| 1543 | 1 | CH₂ | 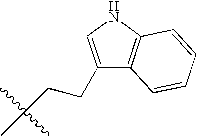 |
| 1544 | 0 | S | 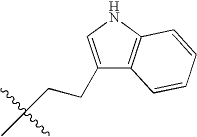 |
| 1545 | 1 | S | 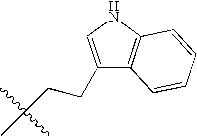 |

Examples for Prolin Mimetics of Formula (XII):

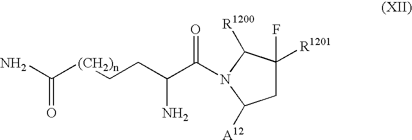

Examples for Prolin Mimetics of Formula (XII):

| Ex. | n | R¹²⁰⁰ | R¹²⁰¹ | A¹² |
|---|---|---|---|---|
| 1600 | 0 | H | F | CN |
| 1601 | 1 | H | F | CN |
| 1602 | 0 | H | F | H |
| 1603 | 1 | H | F | H |
| 1604 | 0 | H | H | H |
| 1605 | 1 | H | H | H |
| 1606 | 0 | CN | F | H |
| 1607 | 1 | CN | F | H |

The present invention provides a compound of the formula

NR¹R²—C(=EWG1)—(CR³R⁴), —CR⁵R⁶—CR⁷R⁸—CR⁹(NR¹⁰OR¹¹)—C(=EWG2)-PM (I)

and especially of the formula

H₂N—CO—CH₂—CH₂—CH(NH₂)—CO-PM

or a pharmaceutically acceptable salt thereof.

The present invention therefore provides a method of treating a condition mediated by modulation of the DPIV or DPIV-like enzyme activity in a subject in need thereof which comprises administering any of the compounds of the present invention or pharmaceutical compositions thereof in a quantity and dosing regimen therapeutically effective to treat the condition. Additionally, the present invention includes the use of the compounds of the present invention, and their corresponding pharmaceutically acceptable acid addition salt forms, for the preparation of a medicament for the prevention or treatment of a condition mediated by modulation of the DPIV activity in a subject.

Indications:

In view of their ability to inhibit DPIV and DPIV-like enzyme activity, the compounds of the present invention, especially the compounds of general formula (I)

NR¹R²—C(=EWG1)—(CR³R⁴)ₙ—CR⁵R⁶—CR⁷R⁸—CR⁹(NR¹⁰R¹¹)—C(=EWG2)-PM (I),

and their corresponding pharmaceutically acceptable acid addition salt forms, are useful for the preparation of a medicament for the treatment of conditions mediated respectively modulated by said enzyme activities in mammals.

Additionally, the capability of the glutaminyl cyclase to control the half life period of the DP IV inhibitor containing a N-terminal glutaminyl or homoglutaminyl residue, respectively, is useful for the preparation of a medicament to definitely control the time of action of the simultaneously administrated DPIV inhibitor. Therefore, the simultaneous administration of both the DPIV inhibitor and the QC inhibitor can be used for the treating conditions mediated respectively modulated by DP IV or DP IV like enzyme activities in mammals for a distinct period of time.

Therefore, the DP IV inhibitors, optionally combined with the QC inhibitors, both disclosed therein, are useful for the preparation of a medicament for the treatment in order to prevent or to alleviate pathological metabolic abnormalities of mammals, preferably of humans, which are related to DP IV or DP IV-like enzyme activity.

Especially, these diseases comprise

metabolic diseases like impaired glucose tolerance, impaired fasting glucose, impaired glucose metabolism, prediabetes, glucosuria, hyperlipidemia, metabolic acidosis, diabetes mellitus, non-insulin dependent diabetes mellitus, diabetic neuropathy and nephropathy and of sequelae caused by diabetes mellitus and obesity;

neurodegenerative diseases; high blood pressure and disturbance of signal action at the cells of the islets of Langerhans and insulin sensitivity in the peripheral tissue in the postprandial phase; the metabolism-related hypertension and cardiovascular sequelae caused by hypertension;

dermal diseases like skin diseases and diseases of the mucosae;

immune and autoimmune disorders, multiple sclerosis, and inflammatory conditions; arthritis;

obesity; allograft transplantation; cancer;,

neuronal disorders as well as psychosomatic, neuropsychiatric and depressive illnesses, such as anxiety, depression, sleep disorders, chronic fatigue, schizophrenia, epilepsy, nutritional disorders, spasm and chronic pain.

The indications above refer each to both acute and chronic form of the disease.

In a more preferred embodiment of this invention, the compounds of the present invention and their corresponding pharmaceutically acceptable acid addition salt forms, improve glucose tolerance by lowering elevated blood glucose levels in response to an oral glucose challenge and, therefore, are useful in treating non-insulin-dependent diabetes mellitus (type 2 diabetes mellitus). The DP IV inhibitors of the present invention are especially used for lowering the blood glucose levels below the glucose concentration characteristic of hyperglycemia in the serum of a mammal, especially of a human, in the case of non-insulin dependent diabetes mellitus.

The compounds and combinations of the present invention are especially useful for the treatment of pathological states, selected from the group consisting of IGT, IFG and IGM, which are characteristic for the prediabetic state.

Galenic Preparations and Formulations:

The compounds of the present invention can be converted into acid addition salts, especially pharmaceutically acceptable acid addition salts.

The method of treating conditions modulated by dipeptidyl peptidase IV and DPIV-like enzymes described in the present invention may also be carried out using a pharmaceutical composition comprising one or more of the compounds as defined herein and a pharmaceutically acceptable carrier. Therefore, the present invention provides, in an further embodiment, formulations for the compounds of the present invention, and their corresponding pharmaceutically acceptable acid addition salt forms, in pharmaceutical compositions.

Preferably these compositions are in unit dosage forms from such as tablets, pills, capsules, powders, granules, sterile parenteral solutions or suspensions, metered aerosol or liquid sprays, drops, ampoules, autoinjector devices or suppositories. The compound may be administered to a patient by any conventional route of administration, including, but not limited to, intravenous, oral, subcutaneous, intramuscular, intradermal, parenteral, intranasal, sublingual or rectal administration, or for administration by inhalation or insufflation.

Compounding techniques: To prepare the pharmaceutical compositions of this invention, one or more compounds of the present invention, especially the DP IV inhibitors according to general formula (I) of the present invention, as well as optionally, the inhibitors of glutaminyl cyclase, and their corresponding pharmaceutically acceptable acid addition salt forms, as the active ingredients, are intimately admixed with a pharmaceutical carrier according to conventional pharmaceutical compounding techniques, which carrier may take a wide variety of forms depending of the form of preparation desired for administration. Compounds of the present invention may also be coupled with soluble polymers as targetable drug carriers.

Homogeneous preparation: For preparing solid compositions such as tablets, the principal active ingredient is ideally mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is ideally dispersed evenly throughout the composition so that the composition may be readily subdivided into equally effective dosage forms such as tablets, pills and capsules. This solid preformulation composition may then be subdivided into unit dosage forms of the type described above containing from about 0.1 to about 1000 mg, preferably from about 5 to about 500 mg of the active ingredient of the present invention.

Concentration and content of active agent: The pharmaceutical compositions herein will contain, per dosage unit, e.g., tablet, capsule, powder, injection, suppository, teaspoonful and the like, of from about 0.01 mg to about 1000 mg (preferably about 5 to about 500 mg) and may be given at a dosage of from about 0.1 to about 300 mg/kg bodyweight per day (preferably 1 to 50 mg/kg per day).

Oral dosage forms: In preparing the compositions in oral dosage form, any of the usual pharmaceutical media may be employed. Compositions suitable for oral administration include solid forms, such as pills, tablets, caplets, capsules (each including immediate release, timed release and sustained release formulations), granules, and powders. For solid oral preparations such as, for example, powders, capsules, gelcaps and tablets, suitable carriers and additives may advantageously include starches, sugars, diluents, granulating agents, lubricants, binders, disintegrating agents and the like. More preferably, for oral administration in the form of a tablet or capsule, the active drug component can be combined with an oral, non-toxic pharmaceutically acceptable inert carrier such as ethanol, glycerol, water and the like.

Coating of tabletts, pills and capsules: Because of their ease in administration, tablets, pills and capsules represent the most advantageous oral dosage unit form, in which case solid pharmaceutical carriers are employed. If desired, the tablets, pills or capsules of the novel composition can be advantageously sugar coated or enteric coated by standard techniques or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids with such materials as shellac, cetyl alcohol and cellulose acetate.

The liquid forms in which the novel compositions of the present invention may be advantageously incorporated for administration orally or by injection include aqueous solutions, suitably flavoured syrups, elixirs, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin. The liquid forms are suitable in flavored suspending or dispersing agents such as the synthetic and natural gums, for example, tragacanth; acacia, methyl-cellulose and the like. Isotonic preparations which generally contain suitable preservatives are employed when intravenous administration is desired.

For liquid oral preparations, such as for example, suspensions, elixirs and solutions, suitable carriers and additives may advantageously include water, glycols, oils, alcohols, flavoring agents, preservatives, coloring agents and the like.

Forms useful for parenteral administration include sterile solutions, emulsions and suspensions.

For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. For parenteral administration, sterile suspensions and solutions are desired. The pharmaceutical compositions herein will contain, per dosage unit, e.g. solution, suspension, emulsion, injection, teaspoonful and the like, an amount of the active ingredient necessary to deliver an effective dose as described above.

Depot formulations for intramuscular injection: Alternatively, the composition may be presented in a form suitable for once-weekly or once-monthly administration; for example, an insoluble salt of the active compound, such as the decanoate salt, may be adapted to provide a depot preparation for intramuscular injection.

Furthermore, compounds for the present invention can be administered in intranasal form via topical use of suitable intranasal vehicles, or via transdermal skin patches well known to those of ordinary skill in that art. To be administered in the form of transdermal delivery system, the dosage administration will, of course, be continuous rather than intermittent throughout the dosage regimen and dosage strength will need to be accordingly modified to obtain the desired therapeutic effects.

The compound of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines using processes well described in the art.

Compounds of this invention may be administered in any of the foregoing compositions and according to dosage regimens established in the art whenever treatment of the addressed disorders is required.

Dosis Regimen and Strength:

Advantageously, compounds of the present invention may be administered in a single daily dose, or the total daily dosage may be administered in divided doses of two, three or four times daily.

The daily dosage of the products may be varied over a wide range from 0.01 to 1.000 mg per adult human per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.1 mg/kg to about 300 mg/kg of body weight per day. Preferably, the range is from about 1 to about 50 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, bioavailability due to the mode of administration, and the advancement of disease condition. In addition, factors associated with the particular patient being treated, including patient age, weight, diet and time of administration, should generally be considered in adjusting dosages.

The dosages, however, may be varied depending upon the requirement of the patients, the severity of the condition being treated and the compound being employed. The use of either daily administration or post-periodic dosing may be employed. Typically the dosage will be regulated by the physician based on the characteristics of the patient, his/her condition and the therapeutic effect desired.

The compounds or compositions of the present invention may be taken before a meal, while taking a meal or after a meal. When taken before a meal the compounds or composition of the present invention an be taken 1 hour, preferably 30 or even 15 or 5 minutes before eating. When taken while eating, the compounds or compositions of the present invention can be mixed into the meal or taken in a separate dosage form as described above. When taken after a meal, the compounds or compositions of the present invention can be taken 5, 15 or 30 minutes or even 1 hour after finishing a meal.

Biochemistry: Inhibition Constants for the DPIV Inhibitors In Vitro and In Vivo

As indicated above, the compounds of the present invention and especially the compounds of the general formula (I), and their corresponding pharmaceutically acceptable acid addition salt forms, are useful in inhibiting DPIV and DPIV-like enzyme activity. The ability of the compounds of the present invention, and their corresponding pharmaceutically acceptable acid addition salt forms to inhibit DPIV and DPIV-like enzyme activity may be demonstrated employing the DPIV activity assay for determination of the Kᵢ-values in vitro and in human plasma.

The ability of the compounds of the present invention, and their corresponding pharmaceutically acceptable acid addition salt forms to inhibit DPIV in vivo may be demonstrated by oral or intravasal administration to Wistar rats. The compounds of the present invention inhibit DPIV activity in vivo after both, oral and intravasal administration to Wistar rats.

Further, the control of the half life period of the DPIV-Inhibitors in vivo by simultaneous administration of DP IV inhibitors and QC inhibitors can be demonstrated, as described in the following example

[image]

Assay:

All assays were performed at 30° C. using the Sunrise reader for microplates (TECAN). Assay mixtures contained the following constituents: 0.4 mM H-Gly-Pro-pNA, 0.65 mU DPIV in 0.04 M Hepes, pH 7.6, containing 0.104 M KCl (FIG. 1, triangles). Additionally, samples contained either

a) 2.6*10⁻5 M glutaminyl thiazolidine (open circles), or

b) 2.6*10⁻⁵ M glutaminyl thiazolidine and 54 mU QC (squares) or

c) 2.6*10⁻⁵ M glutaminyl thiazolidine, 54 mU QC and 0.4 mM 1-benzylimidazole (filled circles).

Reactions were started by addition of H-Gly-Pro-pNA when QC was omitted from the assay.

Otherways, reactions were started by addition of a mixture of H-Gly-Pro-pNA and glutaminyl thiazolidine. Reactions were followed by monitoring the decrease in absorbance at 400 nm.

One unit of QC is defined as the amount of enzyme catalyzing the formation of 1 Amol pGlu-βNA from H-Gln-βNA per minute at 30° C. in samples consisting of 0.2 mM fluorogenic substrate, 0.25 U pyroglutamyl aminopeptidase in 0.2 M Tris/HCl, pH 8.0 containing 20 mM EDTA. One unit of DPIV is defined as the amount of enzyme catalyzing the hydrolysis of 1 μmol H-Gly-Pro-pNA per minute at 30° C. in samples consisting of 0.4 mM substrate in 0.04 M Hepes, pH 7.6 containing 0.104 M KCl.

As can be seen from the absorbance time diagram above, DPIV hydrolyzes H-Gly-Pro-pNA, which does not absorbe at 340 nm (═H-glycyl-prolyl-para-nitroanilide) into H-Gly-Pro-OH and para-nitroaniline, which absorbs radiation of 340 nm; this reaction type is relatively fast and is represented by triangels.

If glutaminyl thiazolidine is added to the mixture of DP IV and H-Gly-Pro-pNA as in case (a), the reaction rate for the hydrolysis reaction decreases due to the competitive inhibition of DPIV by the DP IV inhibitor glutaminyl thiazolidine this reaction demonstrates the inhibitory action of glutaminyl thiazolidine in DP IV and is represented by open circles.

If, additionally in case (b), glutaminyl cyclase is added the DP IV inhibitor glutaminyl thiazolidine is degraded to the pyro-glutaminyl-thiazolidine according to the reaction scheme mentioned above. The pyro-glutaminyl-thiazolidine is formed by the cyclisation reaction of glutaminyl thiazolidine through glutaminyl cyclase (QC) according to the reaction scheme. The cyclic product, pyro-glutaminyl-thiazolidine, is not active as an inhibitor for DP IV. Therefore the DP IV is only inhibited partially by glutaminyl thiazolidine, which is reduced in its concentration by the simultaneously present glutaminyl cyclase to the inactive cyclic pyro-derivate. Thus, the reaction rate for the hydrolysis reaction, represented by squares, is between the uninhibited reaction (triangles) and the strongly inhibited reaction (open circles, case (a)).

If, further additionally in case (c), benzimidazole is added to the reaction mixture, the reaction rate goes down as low as in case (a) where inhibition is only effected by the DP IV inhibitor glutaminyl thiazolidine. This effect can be explained as follows: benzimidazol is an inhibitor of glutaminyl cyclase which is therefore prevented to degrade the DP IV inhibitor glutaminyl thiazolidine to the cyclic pyro-glutamine thiazolidine beeing inactive as a DP IV inhibitor.

Therefore, the concentration of the DP IV inhibitor glutaminyl thiazolindine is maintained in the simiultaneous presence of gluatminyl cyclase (QC) and its inhibitor benzimidazole so as to glutaminyl thiazolidine is capable of inhibiting DP IV to hydrolyse the chromogenic substrate H-Gly-Pro-pNA. Thus, the reaction rate for the hydrolysis reaction in case (c) marked with filled circles is as nearly as low as in case (a).

To summarize, it can be taken from the experiment above, that glutaminyl thiazolidine is degraded to the cyclic pyro-gluatmine derivatie being inactive as a DP IV inhibitor. Thus, the half-life of glutaminyl thiazoldine is reduced in the presence of QC (case (b)) resulting in a higher hydrolyses rate in the substrate compared with case (a) where no QC was present.

Further, it can be concluded from the above experiment that the half life of the DP IV inhibitor glutaminyl thiazolidine—in the presence of the enzyme glutaminyl cyclase , which is naturally present in humans—can be controlled by the addition of the glutaminyl cyclase inhibitor benzimidazole (case (c)). Thus, the hydrolysis reaction rate is decreased in case (c) compared woth case (b), where no glutaminyl cyclase inhibitor such as benzimidazole was present.

Generally spoken, it means, that the addition of a QC inhibitor allows to control the half-life of action of a DP IV inhibitor according to the present invention to inhibit the DP IV enzyme by the mechanism described above. This is an essential aspect of this application.

DPIV is present in a wide variety of mammalian organs and tissues e.g. the intestinal brush-border (Gutschmidt S. et al., “In situ”—measurements of protein contents in the brush border region along rat jejunal villi and their correlations with four enzyme activities. Histochemistry 1981, 72 (3), 467-79), exocrine epithelia, hepatocytes, renal tubuli, endothelia, myofibroblasts (Feller A. C. et al., A monoclonal antibody detecting dipeptidylpeptidase IV in human tissue.

Virchows Arch. A. Pathol. Anat. Histopathol. 1986; 409 (2):263-73), nerve cells, lateral membranes of certain surface epithelia, e.g. Fallopian tube, uterus and vesicular gland, in the luminal cytoplasm of e.g., vesicular gland epithelium, and in mucous cells of Brunner's gland (Hartel S. et al., Dipeptidyl peptidase (DPP) IV in rat organs. Comparison of immunohistochemistry and activity histochemistry. Histochemistry 1988; 89 (2): 151-61), reproductive organs, e.g. cauda epididymis and ampulla, seminal vesicles and their secretions (Agrawal & Vanha-Perttula, Dipeptidyl peptidases in bovine reproductive organs and secretions.

Int. J. Androl. 1986, 9 (6): 435-52). In human serum, two molecular forms of dipeptidyl peptidase are present (Krepela E. et al., Demonstration of two molecular forms of dipeptidyl peptidase IV in normal human serum. Physiol. Bohemoslov. 1983, 32 (6): 486-96). The serum high molecular weight form of DPIV is expressed on the surface of activated T cells (Duke-Cohan J. S. et al., Serum high molecular weight dipeptidyl peptidase IV (CD26) is similar to a novel antigen DPPT-L released from activated T cells. J. Immunol. 1996, 156 (5): 1714-21).

The compounds of the present invention, and their corresponding pharmaceutically acceptable acid addition salt forms are able to inhibit DPIV in vivo. In one embodiment of the present invention, all molecular forms, homologues and epitopes of DPIV from all mammalian tissues and organs, also of those, which are undiscovered yet, are intended to be embraced by the scope of this invention.

Among the rare group of proline-specific proteases, DPIV was originally believed to be the only membrane-bound enzyme specific for proline as the penultimate residue at the amino-terminus of the polypeptide chain. However, other molecules, even structurally non-homologous with the DPIV but bearing corresponding enzyme activity, have been identified recently. DPIV-like enzymes, which are identified so far, are e.g. fibroblast activation protein ax, dipeptidyl peptidase IV β, dipeptidyl aminopeptidase-like protein, N-acetylated α-linked acidic dipeptidase, quiescent cell proline dipeptidase, dipeptidyl peptidase II, attractin and dipeptidyl peptidase IV related protein (DPP 8), and are described in the review article by Sedo & Malik (Sedo & Malik, Dipeptidyl peptidase IV-like molecules: homologous proteins or homologous activities? Biochimica et Biophysica Acta 2001, 36506: 1-10).

Further DPIV-like enzymes are disclosed in WO 01/19866, WO 02/04610, WO 02/34900 and WO02/31134. WO 01/19866 discloses novel human dipeptidyl aminopeptidase (DPP8) with structural und functional similarities to DPIV and fibroblast activation protein (FAP). WO 02/04610 provides reagents, which regulate human dipeptidyl peptidase IV-like enzyme and reagents which bind to human dipeptidyl peptidase IV-like enzyme gene product. These reagents can play a role in preventing, ameliorating, or correcting dysfunctions or diseases including, but not limited to, tumors and peripheral and central nervous system disorders including pain and neurodegenerative disorders. The dipeptidyl peptidase IV-like enzyme of WO 02/04610 is well known in the art. In the Gene Bank data base, this enzyme is registered as KIAA¹⁴⁹² (registration in February 2001, submitted on Apr. 4, 2000, AB040925). WO 02/34900 discloses a dipeptidyl peptidase 9 (DPP9) with significant homology with the amino acid sequences of DPIV and DPP8. WO 02/31134 discloses three DPIV-like enzymes, DPRP1, DPRP2 and DPRP3. Sequence analysis revealed, that DPRP1 is identical to DPP8, as disclosed in WO 01/19866, that DPRP2 is identical to DPP9 and that DPRP3 is identical to KIAA¹⁴⁹² as disclosed in WO 02/04610.

In another preferred embodiment of the present invention, all molecular forms, homologues and epitopes of proteins comprising DPIV-like enzyme activity, from all mammalian tissues and organs, also of those, which are undiscovered yet, are intended to be embraced by the scope of this invention.

In Vivo Tests with Diabetic Zucker Rats

The ability of the compounds of the present invention, and their corresponding pharmaceutically acceptable acid addition salt forms, to improve glucose tolerance in response to an oral glucose 25 challenge, may be measured in diabetic Zucker rats. The method is described in examples 6 and 7. Oral administration of 5 mg/kg b.w., 15 mg/kg and 50 mg/kg b.w. of compounds according to the general formula (I) resulted in a dose dependent lowering of elevated blood glucose levels and thereby in an improvement of glucose tolerance in diabetic Zucker rats.

### EXAMPLES

### Example 1

### Synthesis of Boc-Gln(Trt)-Pro-NH₂

Boc-Gln(Trt)-Pro-NH₂

Di-isopropylamine was added to a solution of H-ProNH₂*HCl in dry CH₂Cl₂until the pH was adjusted to 9. Boc-Gln(Trt)-OSu was added in one portion and the mixture stirred for 16h under an argon atmosphere. The solvent was evaporated and the residue treated in a standard way, i.e. the residue was partioned between ethylacetate and 0.3N KHSO₄solution. The organic layer was further washed with saturated NaHCO₃solution, water and brine. The solution was dried and evaporated at reduced pressure.

### Example 2

### Synthesis of Boc-Gln(Trt)-Pyrr-CN

Boc-Gln(Trt)-Pyrr-CN

Imidazole was added to a solution of Boc-Gln(Trt)-Pro-NH₂ in dry pyridine under an argon atmosphere. The solution was cooled to −35° C., before the dropwise addition of POCl₃. The reaction was stirred at −30° C.-to −20° C. for 60 min. The solution was then evaporated and the crude residue subjected to column chromatography (silica gel) to yield Boc-Gln(Trt)-Pyrr-CN of as a colurless oil.

### Example 3

### Synthesis of H-Gln-Pyrr-CN*TFA

H-Gln-Pyrr-CN*TFA

Deprotection was carried out by stirring with triflouro acetic acid for 60 min. Evaporation and lyophilisation from water afforded 2-(S)cyano-1-glutaminylpyrrolidine as a white solid.

### Example 4

### Kᵢ-Determination

For Ki determination of the compounds of the general formula (I), dipeptidyl peptidase IV from porcine kidney with a specific activity against glycylprolyl-4-nitroaniline of 37.5 U/mg and an enzyme concentration of 1.41 mg/ml in the stock solution was used.

Assay Mixture:

100 ul of a solution containing the compound of the general formula (I) in a concentration range of 1*10⁻5M-1*10⁻⁸M were admixed with 50 Al glycylprolyl-4-nitroaniline in different concentrations (0.4 mM, 0.2 mM, 0.1 mM, 0,05 mM) and 100 Al HEPES (40 mM, pH7.6; ion strength=0.125). The assay mixture was pre-incubated at 30° C. for 30 min After pre-incubation, 20 μl DPIV (1:600 diluted) were added and measurement of yellow color development due to 4-nitroaniline release was performed at 30° C. and 0=405 nm for 10 min using a plate reader (HTS7000 plus, Applied Biosystems, Weiterstadt, Germany).

The Kᵢ-values were calculated using Graphit 4.0.15 (Erithacus Software, Ltd, UK) based on a competitive inhibition of DPIV by the compound of the general formula (I).

### Example 5

### Kᵢ-Determination in Human Plasma

Human Plasma Contains N-Terminal Xaa-Pro Releasing Activity. (definition for Xaa: any Amino Acid, preferably an L-α-amino Acid)

70 μl of a soluation of the compound of the general formula (I) in an concentration range of 1*10⁻⁵M -1*10⁻⁸M were admixed with 50 Al glycylprolyl-4-nitroaniline in different concentrations (0.4 mM, 0.2 mM, 0.1 mM, 0,05 mM) and 100 μl HEPES (40 mM, pH 7.6). The assay mixture was pre-incubated at 30° C. for 5 min and 22 hours respectively. After pre-incubation, 50 Al human plasma were added and measurement of yellow color development due to 4-nitroaniline release was performed at 30° C. and □=405 nm for 10 min using a plate reader (HTS7000 plus, Applied Biosystems, Weiterstadt, Germany).

The Kᵢ-values were calculated using Graphit 4.0.15 (Erithacus Software, Ltd, UK) based on a competitive inhibition of DPIV by the compound of the general formula (I).

### Example 6

### Determination of DPIV Inhibiting Activity of a Compound of the General Formula (I) after Intravasal and Oral Administration to Wistar Rats

Animals

Male Wistar rats (Shoe: Wist(Sho)) with a body weight ranging between 250 and 350 g were purchased from Tierzucht Schbnwalde (Schonwalde, Germany).

Housing Conditions

Animals were single-caged under conventional conditions with controlled temperature (22±20C) on a 12/12hours light/dark cycle (light on at 06:00 AM). Standard pelleted chow (ssniff® Soest, Germany) and tap water acidified with HCl were allowed ad libitum.

Catheter Insertion into Carotid Artery

After ≧one week of adaptation at the housing conditions, catheters were implanted into the carotid artery of Wistar rats under general anaesthesia (i.p. injection of 0.25 ml/kg b.w. Rompun® [2%], BayerVital, Germany and 0.5 ml/kg b.w. Ketamin 10, Atarost GmbH & Co., Twistringen, Germany). The animals were allowed to recover for one week. The catheters were flushed with heparin-saline (100 IU/ml) three times per week.

In case of catheter dysfunction, a second catheter was inserted into the contra-lateral carotid artery of the respective rat. After one week of recovery from surgery, this animal was reintegrated into the study. In case of dysfunction of the second catheter, the animal was withdrawn from the study. A new animal was recruited and the experiments were continued in the planned sequence, beginning at least 7 days after catheter implantation.

Experimental Design

To rats with intact catheter function were administered placebo (1 ml saline, 0.154 mol/l) or 100 mg/kg b.w. of the compound of the general formula (I) via the oral and the intra-vasal (intra-arterial) route.

After overnight fasting, 100 μl samples of heparinised arterial blood were collected at −30, −5, and 0 min The test substance was dissolved freshly in 1.0 ml saline (0.154 mol/l) and was administered at 0 min either orally via a feeding tube (75 mm; Fine Science Tools, Heidelberg, Germany) or via the intra-vasal route. In the case of oral administration, an additional volume of 1 ml saline was injected into the arterial catheter. In the case of intra-arterial administration, the catheter was immediately flushed with 30 Al saline and an additional 1 ml of saline was given orally via the feeding tube.

After application of placebo or the test substances, arterial blood samples were taken at 2.5, 5, 7.5, 10, 15, 20, 40, 60 and 120 min from the carotid catheter of the conscious unrestrained rats.

All blood samples were collected into ice cooled Eppendorf tubes (Eppendorf-Netheler-Hinz, Hamburg, Germany) filled with 10 μl 1M sodium citrate buffer (pH 3.0) for plasma DPIV activity measurement. Eppendorf tubes were centrifuged immediately (12000 rpm for 2 min, Hettich Zentrifuge EBA 12, Tuttlingen; Germany): The plasma fractions were stored on ice until analysis or were frozen at −20° C. until analysis. All plasma samples were labelled with the following data:

Code number

Animal Number

Date of sampling

Time of sampling

Analytical Methods

The assay mixture for determination of plasma DPIV activity consisted of 80 μl reagent and 20 μl plasma sample. Kinetic measurement of the formation of the yellow product 4-nitroaniline from the substrate glycylprolyl-4-nitroaniline was performed at 390 nm for 1 min at 300C after 2 min pre-incubation at the same temperature. The DPIV activity was expressed in mU/ml.

Statistical Methods

Statistical evaluations and graphics were performed with PRISM® 3.02 (GraphPad Software, Inc.). All parameters were analysed in a descriptive manner including mean and SD.

Results

The compounds of the general formula (I) in a dose of 100 mg/kg b.w. vs. placebo inhibited 30 plasma DPIV activity after oral and intra-vasal administration.

### Example 7

### Dose Escalation Study in Fatty Zucker Rats After Oral Administration of a Compound of the General Formula (I)

Animals

N=30 male Zucker rats (fa/fa), mean age 11 weeks (5-12 weeks), mean body weight 350 g (150-400 g), were purchased from Charles River (Sulzfeld, Germany).

After delivery they were kept for >12 weeks until nearly all fatty Zucker rats had the characteristics of manifest diabetes mellitus. A group of N=8 animals were recruited for testing three escalating doses of a compound of the general formula (I) vs. placebo (saline).

Housing Conditions

Animals were single-caged under standardized conditions with controlled temperature (22±20C) on a 12/12hours light/dark cycle (light on at 06:00 AM). Sterile standard pelleted chow (ssniff® Soest, Germany) and tap water acidified with HCl were allowed ad libitum.

Catheterization of Carotid Artery

Fatty Zucker rats of 24-31 weeks (mean: 25 weeks) age, adapted to the housing conditions, were well prepared for the study.

Catheters were implanted into the carotid artery of fatty Zucker rats under general anaesthesia (i.p. injection of 0.25 ml/kg b.w. Rompun® [2%], BayerVital, Germany and 0.5 ml/kg b.w.

Ketamin 10, Atarost GmbH & Co., Twistringen, Germany). The animals were allowed to recover for one week. The catheters were flushed with heparin-saline (100 IU/ml) three times per week.

Experimental Design

Placebo (1 ml saline, 0.154 mol/l) or escalating doses of a compound of the general formula (I) (5, 15 and 50 mg/kg b.w.) were administered to groups of N=8 fatty Zucker rats. 2 mmol of a compound of the general formula (I) were dissolved in 1000 Al DMSO (E. Merck, Darmstadt; Germany [Dimethyl sulfoxide p.a.]).10 ml saline were added and 1 ml aliquots, each containing 0,17 mmol of a compound of the general formula (I), were stored at −20° C. For preparation of the test substance, dose dependent aliquots were diluted in saline.

After overnight fasting, placebo or test substance were administered to the fatty Zucker rats via feeding tube orally (15 G, 75 mm; Fine Science Tools, Heidelberg, Germany) at −10 min An oral glucose tolerance test (OGTT) with 2 g/kg b.w. glucose (40% solution, B. Braun Melsungen, Melsungen, Germany) was administered at ±0 min via a second feeding tube. Venous blood samples from the tail veins were collected at −30 min, -15 min, ±0 min and at 5, 10, 15, 20, 30, 40, 60, 90 and 120 min into 20 Al glass capillaries, which were placed in standard tubes filled with 1 ml solution for blood glucose measurement.

All blood samples were labelled with the following data:

Code number

Animal Number

Date of sampling

Time of sampling

Analytical Methods

Glucose levels were measured using the glucose oxidase procedure (Super G Glucose analyzer; Dr. Muiller Geraitebau, Freital, Germany).

Statistical Methods

Statistical evaluations and graphics were performed with PRISM® 3.02 (GraphPad Software, Inc.). All parameters were analysed in a descriptive manner including mean and SD.

Effect of Medication on Glucose Tolerance

The placebo treated diabetic Zucker rats showed a strongly elevated blood glucose excursion indicating glucose intolerance of manifest diabetes mellitus. Administration of 5 mg/kg b.w. of the compound of the general formula (I) resulted in a limited improvement of glucose tolerance in diabetic Zucker rats. Significant lowering of elevated blood glucose levels and improvement of glucose tolerance was achieved after administration of 15 mg/kg and 50 mg/kg b.w. of the compound according to general formula (I).

### Example 8

### In Vivo Inactivation of a Compound of the General Formula (I) After Oral Administration to Wistar Rats

Animals/Experimental Design

A compound of the general formula (I) was administered to Wistar rats orally as described in example 9. to determine the conversion to the corresponding cyclic inactive pyro-glutamine derivative compound.

Analytical methods

After application of placebo or a compound of the general formula (I), arterial blood samples were taken at 2.5, 5, 7.5, 10, 15, 20, 40, 60 and 120 min from the carotid catheter of the conscious unrestrained rats to determine the formation of degradation products of the compound of the general formula, the corresponding cyclic inactive pyro-glutamine derivative compound. For analysis, simple solid phase extraction procedure on C₁₈ cartridges was used to isolate the compounds of interest from the plasma. The extracts were analysed using reversed-phase liquid chromatography on Lichrospher 60, RP Select B column hyphenated with tandem mass spectrometry operating in the APCI positive mode. An internal standard method was used for quantification.

Results

After oral administration of a compound of the general formula (I) to Wistar rats, a degradation of the compound was found. Using LC/MS, the degradation product could be indentified as the corresponding pyroglutaminyl derivative of the compound of the general formula (I).

### Example 9

### Assays for Glutaminyl Cyclase Activity

Fluorometric Assays

All measurements were performed with a BioAssay Reader HTS-7000Plus for microplates (Perkin Elmer) at 30° C. QC activity was evaluated fluorometrically using H-Gln-,A. The samples consisted of 0.2 mM fluorogenic substrate, 0.25 U pyroglutamyl aminopeptidase (Unizyme, Horsholm, Denmark) in 0.2 M Tris/HCl, pH 8.0 containing 20 mM EDTA and an appropriately diluted aliquot of QC in a final volume of 250 μl. Excitation/emission wavelengths were 320/410 nm. The assay reactions were initiated by addition of glutaminyl cyclase. QC activity was determined from a standard curve of β-naphthylamine under assay conditions. One unit is defined as the amount of QC catalyzing the formation of 1 μmol pGlu-βNA from H-Gln-βNA per minute under the described conditions.

In a second fluorometric assay, QC was activity was determined using H-Gln-AMC as substrate. Reactions were carried out at 30° C. utilizing the NOVOStar reader for microplates (BMG labtechnologies). The samples consisted of varying concentrations of the fluorogenic substrate, 0.1 U pyroglutamyl aminopeptidase (Qiagen) in 0.05 M Tris/HCl, pH 8.0 containing 5 mM EDTA and an appropriately diluted aliquot of QC in a final volume of 250 μl. Excitation/emission wavelengths were 380/460 nm. The assay reactions were initiated by addition of glutaminyl cyclase. QC activity was determined from a standard curve of 7-amino-4-methylcoumarin under assay conditions. The kinetic data were evaluated using GraFit software.

Spectrophotometric Assay of QC

This novel assay was used to determine the kinetic parameters for most of the QC substrates. QC activity was analyzed spectrophotometrically using a continuous method, that was derived by adapting a previous discontinuous assay (Bateman, R. C. J. 1989 J Neurosci Methods 30, 23-28) utilizing glutamate dehydrogenase as auxiliary enzyme. Samples consisted of the respective QC substrate, 0.3 mM NADH, 14 mM x-Ketoglutaric acid and 30 U/ml glutamate dehydrogenase in a final volume of 250 μl. Reactions were started by addition of QC and perused by monitoring of the decrease in absorbance at 340 nm for 8-15 min. Typical time courses of product formation are presented in FIG. 1.

The initial velocities were evaluated and the enzymatic activity was determined from a standard curve of ammonia under assay conditions. All samples were measured at 30° C., using either the SPECTRAFluor Plus or the Sunrise (both from TECAN) reader for microplates. Kinetic data was evaluated using GraFit software.

Inhibitor Assay

For inhibitor testing, the sample composition was the same as described above, except of the putative inhibitory compound added. For a rapid test of QC-inhibition, samples contained 4 mM of the respective inhibitor and a substrate concentration at 1 KM. For detailed investigations of the inhibition and determination of Kᵢ-values, influence of the inhibitor on the auxiliary enzymes was investigated first. In every case, there was no influence on either enzyme detected, thus enabling the reliable determination of the QC inhibition. The inhibitory constant was evaluated by fitting the set of progress curves to the general equation for competitive inhibition using GraFit software.

1. A compound of the general formula (I)
NR¹R²—C(=EWG1)—(CR³R⁴)ₙ—CR⁵R⁶—CR⁷R⁸—CR⁹(NR¹⁰OR¹¹)—C(=EWG2)-PM (I)
wherein n is 0 or 1;
wherein R¹R², R³, R⁴, R⁵, R⁶, R⁷, R⁸, R⁹, R¹⁰, and R¹¹, independently of each other, are
a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R²⁰), a boronic acid group (—B(OH)₂), a cyano group (—C≡N), a carboxylic acid group (—COOH), a carboxylic acid ester group (—COOR²), a carboxylic acid anhydride group (—CO—O—CO—R²²), a hydroxamic acid group (—CO—NH(OH)), a N-substituted hydroxamic acid group (—CO—NR² (OH)), a O-substituted hydroxamic acid group (—CO—NH(OR²⁴)), a carboxamide group (—CO—NH₂), a N-substituted or N,N-disubstituted carboxylic acid amide group, (—CO—NHR²¹; —CO—NR²⁶R²⁷), an amido group (—HN—CO—R²⁸), a sulfonic acid group (—SO₃H), a sulfonamide group (—SO₂—NH₂), a N-substituted or N,N-disubstituted sulfonamide group (—SO₂—NHR²⁹; —SO₂—NR³⁰R³¹), an amidosulfone group (—NH—SO₂—R³²), a sulfone group (—SO₂—R³³), a phosphoric acid group (—OP(═O)(OH)₂), a phosphoric acid ester group (—OP(═O)(OR³⁴)(OR³⁵)), a phosphonic acid group (—P(═O)(OH)₂), an phosphonic acid ester group (—P(═O)(OR³⁶)(OR³⁷)), a halogen atom, a trifluormethyl group (—CF₃), a thiol group (—SH); a thioether group (—S—R³⁸), a hydroxy group (—OH); an alkoxy group (—O—R³⁹), a tetrazole group, an amino group (—NH₂), or a N-substituted or N,N-disubstituted amino group (—NHR⁴⁰; —NR⁴¹R⁴²);
which each independently can be substituted with one or more substituents, which can be the same or different; and,
wherein optionally, any two of the groups R¹, R², R³, R⁴, R⁵, R⁶, R⁷, R⁸, R⁹, R¹⁰, and R¹¹, as well the pairs R²⁶/R²⁷, R³⁰/R³¹, R³⁴/R³⁵, R³⁶/R³⁷ and R⁴¹/R⁴², independenly of each other, may form a part of a ring; and
wherein the substituents R²⁰, R²¹, R²², R²³, R²⁴, R²⁵, R²⁶, R²⁷, R²⁸, R²⁹, R³⁰, R³¹, R³², R³³, R³⁴R³⁵, R³⁶, R³⁷, R³⁸, R³⁹, R⁴⁰, R⁴¹, and R⁴² independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group; and
wherein EWG1 and EWG2 are each independently an electron withdrawing group and;
wherein the group PM
has the formula (II) 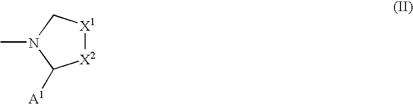
wherein X¹ is CR⁵¹R⁵², O, S, SO, SO₂or NR⁵³; and
wherein X² is CR⁵⁴R⁵⁵, O, S, SO, SO₂, or NR⁵⁶; and
wherein R⁵¹, R⁵², R⁵³, R⁵⁴, R⁵⁵, and R⁵⁶, independently of each other, are
a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁶⁰), a boronic acid group (—B(OH)₂), a cyano group (—C≡N), a carboxylic acid group (—COOH), a carboxylic acid ester group (—COOR⁶¹), a carboxylic acid anhydride group (—CO—O—CO—R⁶²), a hydroxamic acid group (—CO—NH(OH)), a N-substituted hydroxamic acid group (—CO—NR⁶³(OH)), a O-substituted hydroxamic acid group (—CO—NH(OR⁶⁴)), a carboxamide group (—CO—NH₂), a N-substituted or N,N-disubstituted carboxylic acid amide group, (—CO—NHR⁶⁵; —CO—NR⁶⁶R⁶⁷), an amido group (—HN—CO—R⁶⁸), a sulfonic acid group (—SO₃H), a sulfonamide group (—SO₂—NH₂), a N-substituted or N,N-disubstituted sulfonamide group (—SO₂—NHR⁶⁹; —SO₂—NR⁷OR⁷¹), an amidosulfone group (—NH—SO₂—R⁷²), a sulfone group (—SO₂—R⁷³), a phosphoric acid group (—OP(═O)(OH)₂), a phosphoric acid ester group (—OP(═O)(OR⁷⁴)(OR⁷⁵)), a phosphonic acid group (—P(═O)(OH)₂), an phosphonic acid ester group (—P(═O)(OR⁷⁶)(OR⁷⁷)), a halogen atom, a trifluormethyl group (—CF₃), a thiol group (—SH); a thioether group (—S—R⁷⁸), a hydroxy group (—OH); an alkoxy group (—O—R⁷⁹), a tetrazole group, an amino group (—NH₂), or a N-substituted or N,N-disubstituted amino group (—NHR⁸⁰; —NR⁸¹R⁸²); and
which, independently of each other, can be substituted with one or more substituents, which can be the same or different; and,
wherein optionally, any two of the groups R⁵¹, R⁵², R⁵³, R⁵⁴, R⁵⁵, and R⁵⁶, if present, as well as the pairs R⁶⁶/R⁶⁷, R⁷⁰/R⁷¹, R⁷⁴/R⁷⁵, R⁷⁶/R⁷⁷ and R⁸¹/R⁸², independently of each other, may form a part of a ring; and
wherein the substituents R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³R⁷⁴, R⁷⁵, R⁷⁶, R⁷⁷, R⁷⁸, R⁷⁹, R⁸⁰, R⁸¹, and R⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group; and
wherein A¹ is
a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R¹⁰⁰), a boronic acid group (—B(OH)₂), a cyano group (—C≡N), a carboxylic acid group (—COOH), a carboxylic acid ester group (—COOR¹⁰¹), a carboxylic acid anhydride group (—CO—O—CO—R¹⁰²), a hydroxamic acid group (—CO—NH(OH)), a N-substituted hydroxamic acid group (—CO—NR¹⁰³(OH)), a O-substituted hydroxamic acid group (—CO—NH(OR¹⁰⁴)), a carboxamide group (—CO—NH₂), a N-substituted or N,N-disubstituted carboxylic acid amide group, (—CO—NHR¹⁰⁵; —CO—NR⁰⁶, R⁰⁷), an amido group (—HN—CO—R¹⁰⁸), a sulfonic acid group (—SO₃H), a sulfonamide group (—SO₂—NH₂), a N-substituted or N,N-disubstituted sulfonamide group (—SO₂—NHR¹⁰⁹; —SO₂—NR¹¹⁰R¹¹¹), an amidosulfone group (—NH—SO₂—R¹¹²), a sulfone group (—SO₂—R¹¹³), a phosphoric acid group (—OP(═O)(OH)₂), a phosphoric acid ester group (—OP(═O)(OR¹¹⁴)(OR¹¹⁵)), a phosphonic acid group (—P(═O)(OH)₂), an phosphonic acid ester group (—P(═O)(OR¹¹⁶)(OR¹⁷)), a halogen atom, a trifluormethyl group (—CF₃), a thiol group (—SH); a thioether group (—S—R¹¹⁸), a hydroxy group (—OH); an alkoxy group (—O—R¹¹⁹), a tetrazole group, an amino group (—NH₂), or a N-substituted or N,N-disubstituted amino group (—NHR²⁰; —NR¹²¹R¹²²); and
which, independently of each other, can be substituted with one or more substituents, which can be the same or different; and,
wherein optionally, the pairs R¹⁰⁶/R⁰⁷, R¹¹⁰/R¹¹¹, R¹⁴/R¹⁵, R⁶/R¹¹⁷ and R¹²¹/R¹²², independently of each other, may form a part of a ring; and
wherein the substituents R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹, R¹¹², R¹¹³, R¹¹⁴, R¹¹⁵, R¹¹⁶, R¹¹⁷, R¹¹⁸, R¹¹⁹, R¹²⁰, R¹²¹, and R¹²², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (IfI) 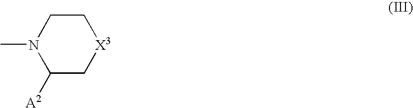
wherein X³ is CR¹³¹R¹³², O, S, SO, SO₂, or NR¹³³; and
wherein R¹³¹, R¹³², and R¹³³, independently of each other, are
a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R¹⁴⁰), a boronic acid group (—B(OH)₂), a cyano group (—C≡N), a carboxylic acid group (—COOH), a carboxylic acid ester group (—COOR¹⁴¹), a carboxylic acid anhydride group (—CO—O—CO—R¹⁴²), a hydroxamic acid group (—CO—NH(OH)), a N-substituted hydroxamic acid group (—CO—NR¹⁴³(OH)), a O-substituted hydroxamic acid group (—CO—NH(OR¹⁴⁴)), a carboxamide group (—CO—NH₂), a N-substituted or N,N-disubstituted carboxylic acid amide group, (—CO—NHR¹⁴⁵; —CO—NR¹⁴⁶, R¹⁴⁷), an amido group (—HN—CO—R¹⁴⁸), a sulfonic acid group (—SO₃H), a sulfonamide group (—SO₂—NH₂), a N-substituted or N,N-disubstituted sulfonamide group (—SO₂—NHR¹⁴⁹; —SO₂—NR¹⁵⁰R¹⁵¹), an amidosulfone group (—NH—SO₂—R¹⁵²), a sulfone group (—SO₂—R¹⁵³), a phosphoric acid group (—OP(═O)(OH)₂), a phosphoric acid ester group (—OP(═O)(OR¹⁵⁴)(OR¹⁵⁵)), a phosphonic acid group (—P(═O)(OH)₂), an phosphonic acid ester group (—P(═O)(OR¹⁵⁶)(OR¹⁵⁷)), a halogen atom, a trifluormethyl group (—CF₃), a thiol group (—SH); a thioether group (—S—R¹⁵⁸), a hydroxy group (—OH); an alkoxy group (—O—R¹⁵⁹), a tetrazole group, an amino group (—NH₂), or a N-substituted or N,N-disubstituted amino group (—NHR¹⁶⁰; —NR¹⁶¹R¹⁶²); and
which, independently of each other, can be substituted with one or more substituents, which can be the same or different; and,
wherein optionally, the the pair R¹³¹/R¹³², if present, as well the pairs R¹⁴⁶/R¹⁴⁷, R¹⁵⁰/R¹⁵¹, R¹⁵⁴/R¹⁵⁵, R¹⁵⁶/R¹⁵⁷ and R¹⁶¹/R¹⁶², independenly of each other, may form a part of a ring; and
wherein the substituents R¹⁴⁰, R¹⁴², R¹⁴³, R¹⁴⁴, R¹⁴⁵, R¹⁴⁶, R¹⁴⁷, R¹⁴⁸, R¹⁴⁹, R¹⁵⁰, R¹⁵¹, R¹⁵², R¹⁵³, R¹⁵⁴, R¹⁵⁵, R¹⁵⁶, R¹⁵⁷, R¹⁵⁸, R¹⁵⁹, R¹⁶⁰, R¹⁶¹, and R¹⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
wherein A² is
a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R¹⁸⁰), a boronic acid group (—B(OH)₂), a cyano group (—C≡N), a carboxylic acid group (—COOH), a carboxylic acid ester group (—COOR¹⁸¹), a carboxylic acid anhydride group (—CO—O—CO—R¹⁸²), a hydroxamic acid group (—CO—NH(OH)), a N-substituted hydroxamic acid group (—CO—NR¹⁸³(OH)), a O-substituted hydroxamic acid group (—CO—NH(OR¹⁸⁴)), a carboxamide group (—CO—NH₂), a N-substituted or N,N-disubstituted carboxylic acid amide group, (—CO—NHR¹⁸⁵; —CO—NR¹⁸⁶R¹⁸⁷), an amido group (—HN—CO—R¹⁸⁸), a sulfonic acid group (—SO₃H), a sulfonamide group (—SO₂—NH₂), a N-substituted or N,N-disubstituted sulfonamide group (—SO₂—NHR¹⁸⁹; —SO₂—NR¹⁹⁰R¹⁹¹), an amidosulfone group (—NH—SO₂—R¹¹²), a sulfone group (—SO₂—R¹⁹³), a phosphoric acid group (—OP(═O)(OH)₂), a phosphoric acid ester group (—OP(═O)(OR⁹⁴)(OR⁹⁵)), a phosphonic acid group (—P(═O)(OH)₂), an phosphonic acid ester group (—P(═O)(OR¹⁹⁶)(OR¹⁹⁷)), a halogen atom, a trifluormethyl group (—CF₃), a thiol group (—SH); a thioether group (—S—R¹⁹⁸), a hydroxy group (—OH); an alkoxy group (—O—R¹⁹⁹), a tetrazole group, an amino group (—NH₂), or a N-substituted or N,N-disubstituted amino group (—NHR²⁰⁰; —NR²⁰¹R²⁰²); and
which, independently of each other, can be substituted with one or more substituents, which can be the same or different; and,
wherein optionally, the pairs R¹⁸⁶/R⁸⁷, R¹⁹⁰/R¹⁹¹, R¹⁹⁴/R¹⁹⁵, R⁹⁶/R⁹⁷ and R²⁰¹/R²⁰² independenly of each other, may form a part of a ring; and
wherein the substituents R¹⁸⁰, R¹⁸¹, R¹⁸², R¹⁸³, R¹⁸⁴, R¹⁸⁵, R¹⁸⁶, R¹⁸⁷, R¹⁸⁸, R¹⁸⁹, R¹⁹⁰ R¹⁹¹, R¹⁹², R¹⁹³, R¹⁹⁴, R¹⁹⁵, R¹⁹⁶, R¹⁹⁷, R¹⁹⁸, R¹⁹⁹, R²⁰⁰, R²⁰¹, and R²⁰², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (IV) 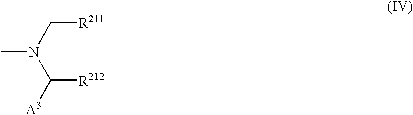
wherein R²¹¹ and R²¹², independently of each other, are
a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R²²⁰), a boronic acid group (—B(OH)₂), a cyano group (—C≡N), a carboxylic acid group (—COOH), a carboxylic acid ester group (—COOR²²¹), a carboxylic acid anhydride group (—CO—O—CO—R²²²), a hydroxamic acid group (—CO—NH(OH)), a N-substituted hydroxamic acid group (—CO—NR²²³(OH)), a O-substituted hydroxamic acid group (—CO—NH(OR²²⁴)), a carboxamide group (—CO—NH₂), a N-substituted or N,N-disubstituted carboxylic acid amide group, (—CO—NHR²²⁵; —CO_NR²²⁶R²²⁷), an amido group (—HN—CO—R²²¹), a sulfonic acid group (—SO₃H), a sulfonamide group (—SO₂—NH₂), a N-substituted or N,N-disubstituted sulfonamide group (—SO₂—NHR²²⁹; —SO₂—NR²³⁰R²³¹), an amidosulfone group (—NH—SO₂—R²³²), a sulfone group (—SO₂—R²³³), a phosphoric acid group (—OP(═O)(OH)₂), a phosphoric acid ester group (—OP(═O)(OR²³⁴)(OR²³⁵)), a phosphonic acid group (—P(═O)(OH)₂), an phosphonic acid ester group (—P(═O)(OR²³⁶)(OR²³⁷)), a halogen atom, a trifluormethyl group (—CF₃), a thiol group (—SH); a thioether group (—S—R²³⁸), a hydroxy group (—OH); an alkoxy group (—O—R²³⁹), a tetrazole group, an amino group (—NH₂), or a N-substituted or N,N-disubstituted amino group (—NHR²⁴⁰; —NR²⁴¹R²⁴²); and
which, independently of each other, can be substituted with one or more substituents, which can be the same or different; and,
wherein optionally, the pairs R²²⁶/R²²⁷, R²³⁰/R²³¹, R²³⁴/R²³⁵, R²³⁶/R²³⁷ and R²⁴¹/R²⁴² independenly of each other, may form a part of a ring; and
wherein the substituents R²²⁰, R²²¹, R²²², R²²³, R²²⁴, R²²⁵, R²²⁶, R²²⁷, R²²⁸, R²²⁹, R²³⁰, R²³¹, R²³², R²³³, R²³⁴, R²³⁵, R²³⁶, R²³⁷, R²³⁸, R²³⁹, R²⁴⁰, R²⁴¹, and R²⁴², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
wherein A³ is
a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R²⁶⁰), a boronic acid group (—B(OH)₂), a cyano group (—C≡N), a carboxylic acid group (—COOH), a carboxylic acid ester group (—COOR²⁶¹), a carboxylic acid anhydride group (—CO—O—CO—R²⁶²), a hydroxamic acid group (—CO—NH(OH)), a N-substituted hydroxamic acid group (—CO—NR²⁶³(OH)), a O-substituted hydroxamic acid group (—CO—NH(OR²⁶⁴)), a carboxamide group (—CO—NH₂), a N-substituted or N,N-disubstituted carboxylic acid amide group, (—CO—NHR²⁶⁵; —CO—NR²⁶⁶R²⁶⁷), an amido group (—HN—CO—R²⁶⁸), a sulfonic acid group (—SO₃H), a sulfonamide group (—SO₂—NH₂), a N-substituted or N,N-disubstituted sulfonamide group (—SO₂—NHR²⁶⁹; —SO₂—NR²⁷⁰R²⁷¹), an amidosulfone group (—NH—SO₂—R²⁷²), a sulfone group (—SO₂—R²⁷³), a phosphoric acid group (—OP(═O)(OH)₂), a phosphoric acid ester group (—OP(═O)(OR²⁷⁴)(OR²⁷⁵)), a phosphonic acid group (—P(═O)(OH)₂), an phosphonic acid ester group (—P(═O)(OR²⁷⁶)(OR²⁷⁷)), a halogen atom, a trifluormethyl group (—CF₃), a thiol group (—SH); a thioether group (—S—R²⁷⁸), a hydroxy group (—OH); an alkoxy group (—O—R²⁷⁹), a tetrazole group, an amino group (—NH₂), or a N-substituted or N,N-disubstituted amino group (—NHR²⁸⁰; —NR²⁸¹R²⁸²); and
which, independently of each other, can be substituted with one or more substituents, which can be the same or different; and,
wherein optionally, the pairs R²⁶⁶/R²⁶⁷, R²⁷⁰/R²⁷¹, R²⁷⁴/R²⁷⁵, R²⁷⁶/R²⁷⁷ and R²⁸¹/R²⁸² independenly of each other, may form a part of a ring; and
wherein the substituents R²⁶⁰, R²⁶¹, R²⁶², R²⁶³, R²⁶⁴, R²⁶⁵, R²⁶⁶, R²⁶⁷, R²⁶⁸, R²⁶⁹, R^{270 R}²⁷¹, R²⁷², R²⁷³, R²⁷⁴, R²⁷⁵, R²⁷⁶, R²⁷⁷, R²⁷⁸, R²⁷⁹, R²⁸⁰, R²⁸¹, and R²⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (V) 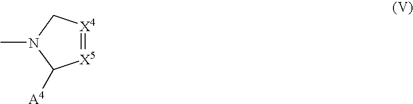
wherein X⁴ is CR²⁹¹ or N; and
wherein X⁵ is CR²⁹² or N; and
wherein R²⁹¹ and R²⁹², independently of each other, are
a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R³⁰⁰), a boronic acid group (—B(OH)₂), a cyano group (—C≡N), a carboxylic acid group (—COOH), a carboxylic acid ester group (—COOR³⁰¹), a carboxylic acid anhydride group (—CO—O—CO—R³⁰²), a hydroxamic acid group (—CO—NH(OH)), a N-substituted hydroxamic acid group (—CO—NR³⁰³(OH)), a O-substituted hydroxamic acid group (—CO—NH(OR³⁰⁴)), a carboxamide group (—CO—NH₂), a N-substituted or N,N-disubstituted carboxylic acid amide group, (—CO—NHR³⁰⁵; —CO—NR³⁰⁶, R³⁰⁷), an amido group (—HN—CO—R³⁰⁸), a sulfonic acid group (—SO₃H), a sulfonamide group (—SO₂—NH₂), a N-substituted or N,N-disubstituted sulfonamide group (—SO₂—NHR³⁰⁹; —SO₂—NR³¹⁰R³¹¹), an amidosulfone group (—NH—SO₂—R³¹²), a sulfone group (—SO₂—R³¹³), a phosphoric acid group (—OP(═O)(OH)₂), a phosphoric acid ester group (—OP(═O)(OR³¹⁴)(OR³¹⁵)), a phosphonic acid group (—P(═O)(OH)₂), an phosphonic acid ester group (—P(═O)(OR³¹⁶)(OR³¹⁷)), a halogen atom, a trifluormethyl group (—CF₃), a thiol group (—SH); a thioether group (—S—R³¹⁸), a hydroxy group (—OH); an alkoxy group (—O—R³¹⁹), a tetrazole group, an amino group (—NH₂), or a N-substituted or N,N-disubstituted amino group (—NHR³²⁰; —NR³²R³²²); and
which, independently of each other, can be substituted with one or more substituents, which can be the same or different; and,
wherein optionally, the the pair R²⁹¹/R²⁹², if present, as well the pairs R³⁰⁶/R³⁰⁷, R³¹⁰/R³¹¹, R³¹⁴/R³¹⁵, R³¹⁶/R³¹⁷ and R³²¹/R³²², independenly of each other, may form a part of a ring; and
wherein the substituents R³⁰⁰, R³⁰¹, R³⁰², R³⁰³, R³⁰⁴, R³⁰⁵, R³⁰⁶, R³⁰⁷, R³⁰⁸, R³⁰⁹, R³¹⁰, R³¹¹, R³¹², R³¹³, R³¹⁴, R³¹⁵, R³¹⁶, R³¹⁷, R³¹⁸, R³¹⁹, R³²⁰, R³²¹, and R³²², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
wherein A⁴ is
a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R³⁴⁰), a boronic acid group (—B(OH)₂), a cyano group (—C≡N), a carboxylic acid group (—COOH), a carboxylic acid ester group (—COOR³⁴¹), a carboxylic acid anhydride group (—CO—O—CO—R³⁴²), a hydroxamic acid group (—CO—NH(OH)), a N-substituted hydroxamic acid group (—CO—NR³⁴³(OH)), a O-substituted hydroxamic acid group (—CO—NH(OR³⁴⁴)), a carboxamide group (—CO—NH₂), a N-substituted or N,N-disubstituted carboxylic acid amide group, (—CO—NHR³⁴⁵; —CO—NR³⁴⁶R³⁴⁷), an amido group (—HN—CO—R³⁴⁸), a sulfonic acid group (—SO₃H), a sulfonamide group (—SO₂—NH₂), a N-substituted or N,N-disubstituted sulfonamide group (—SO₂—NHR³⁴⁹; —SO₂—NR³⁵OR³⁵¹), an amidosulfone group (—NH—SO₂—R³⁵²), a sulfone group (—SO₂—R³⁵³), a phosphoric acid group (—OP(═O)(OH)₂), a phosphoric acid ester group (—OP(═O)(OR³⁵⁴)(OR³⁵⁵)), a phosphonic acid group (—P(═O)(OH)₂), an phosphonic acid ester group (—P(═O)(OR³⁵⁶)(OR³⁵⁷)), a halogen atom, a trifluormethyl group (—CF₃), a thiol group (—SH); a thioether group (—S—R³⁵⁸), a hydroxy group (—OH); an alkoxy group (—O—R³⁵⁹), a tetrazole group, an amino group (—NH₂), or a N-substituted or N,N-disubstituted amino group (—NHR³⁶⁰; —NR³⁶¹R³⁶²); and
which, independently of each other, can be substituted with one or more substituents, which can be the same or different; and,
wherein optionally, the pairs R³⁴⁶/R³⁴⁷, R³⁵⁰/R³⁵, R³⁵⁴/R³⁵⁵, R³⁵⁶/R³⁵⁷ and R³⁶/R³⁶² independenly of each other, may form a part of a ring; and
wherein the substituents R³⁴⁰, R³⁴¹, R³⁴², R³⁴³, R³⁴⁴, R³⁴⁵, R³⁴⁶, R³⁴⁷, R³⁴⁸, R³⁴⁹, R³⁵⁰, R³⁵¹, R³⁵², R³⁵³, R³⁵⁴, R³⁵⁵, R³⁵⁶, R³⁵⁷, R³⁵⁸, R³⁵⁹, R³⁶⁰, R³⁶¹ and R³⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (VI) 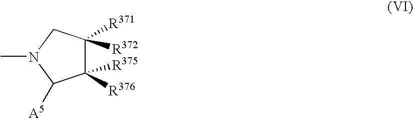
wherein R³⁷¹, R³⁷², R³⁷³ and R³⁷⁴ independently of each other, a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R³⁸⁰), a boronic acid group (—B(OH)₂), a cyano group (—C≡N), a carboxylic acid group (—COOH), a carboxylic acid ester group (—COOR³⁸¹), a carboxylic acid anhydride group (—CO—O—CO—R³⁸²), a hydroxamic acid group (—CO—NH(OH)), a N-substituted hydroxamic acid group (—CO—NR³⁸³(OH)), a O-substituted hydroxamic acid group (—CO—NH(OR³⁸⁴)), a carboxamide group (—CO—NH₂), a N-substituted or N,N-disubstituted carboxylic acid amide group, (—CO—NHR³⁸⁵; —CO—NR³⁸⁶R³⁸⁷), an amido group (—HN—CO—R³⁸⁸), a sulfonic acid group (—SO₃H), a sulfonamide group (—SO₂—NH₂), a N-substituted or N,N-disubstituted sulfonamide group (—SO₂—NHR³⁸⁹; —SO₂—NR³⁹OR³⁹¹), an amidosulfone group (—NH—SO₂—R³⁹²), a sulfone group (—SO₂—R³⁹³), a phosphoric acid group (—OP(═O)(OH)₂), a phosphoric acid ester group (—OP(═O)(OR³⁹⁴)(OR³⁹⁵)), a phosphonic acid group (—P(═O)(OH)₂), an phosphonic acid ester group (—P(═O)(OR³⁹⁶)(OR³⁹⁷)), a halogen atom, a trifluormethyl group (—CF₃), a thiol group (—SH); a thioether group (—S—R³⁹⁸), a hydroxy group (—OH); an alkoxy group (—O—R³⁹⁹), a tetrazole group, an amino group (—NH₂), or a N-substituted or N,N-disubstituted amino group (—NHR⁰⁰; —NR⁴⁰¹R⁴⁰²); and
which, independently of each other, can be substituted with one or more substituents, which can be the same or different; and,
wherein optionally, any two of the groups R³⁷¹, R³⁷², R³⁷⁵, and R³⁷⁶, as well as the pairs R³⁸⁶/R³⁸⁷, R³⁹⁰/R³⁹¹, R³⁹⁴/R³⁹⁵, R³⁹⁶/R³⁹⁷ and R⁴⁰¹/R⁴⁰², independenly of each other, may form a part of a ring; and
wherein the substituents R³⁸⁰, R³⁸¹, R³⁸², R³⁸³, R³⁸⁴, R³⁸⁵, R³⁸⁶, R³⁸⁷, R³⁸⁸, R³⁸⁹, R³⁹⁰, R³⁹¹, R³⁹², R³⁹³, R³⁹⁴, R³⁹⁵, R³⁹⁶, R³⁹⁷, R³⁹⁸, R³⁹⁹, R⁴⁰⁰, R⁴⁰¹, and R⁴⁰², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group; or
alternatively; the two groups R³⁷¹ and R³⁷² can be together an oxo (═O) or hydroxyimino (═N—OH) group; and
alternatively; the two groups R³⁷ and R³⁷⁶ can be together an oxo (═O) or hydroxyimino (═N—OH) group; and
wherein A⁵ is
a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁴²⁰), a boronic acid group (—B(OH)₂), a cyano group (—C≡N), a carboxylic acid group (—COOH), a carboxylic acid ester group (—COOR⁴²¹), a carboxylic acid anhydride group (—CO—O—CO—R⁴²²), a hydroxamic acid group (—CO—NH(OH)), a N-substituted hydroxamic acid group (—CO—NR⁴²³(OH)), a O-substituted hydroxamic acid group (—CO—NH(OR⁴²⁴)), a carboxamide group (—CO—NH₂), a N-substituted or N,N-disubstituted carboxylic acid amide group, (—CO—NHR⁴²⁵; —CO—NR⁴²⁶, R⁴²⁷), an amido group (—HN—CO—R⁴²⁸), a sulfonic acid group (—SO₃H), a sulfonamide group (—SO₂—NH₂), a N-substituted or N,N-disubstituted sulfonamide group (—SO₂—NHR⁴²⁹; —SO₂—NR⁴³OR⁴³¹), an amidosulfone group (—NH—SO₂—R⁴³²), a sulfone group (—SO₂—R⁴³³), a phosphoric acid group (—OP(═O)(OH)₂), a phosphoric acid ester group (—OP(═O)(OR⁴³⁴)(OR⁴³⁵)), a phosphonic acid group (—P(═O)(OH)₂), an phosphonic acid ester group (—P(═O)(OR⁴³⁶)(OR⁴³⁷)), a halogen atom, a trifluormethyl group (—CF₃), a thiol group (—SH); a thioether group (—S—R⁴³⁸), a hydroxy group (—OH); an alkoxy group (—O—R⁴³⁹), a tetrazole group, an amino group (—NH₂), or a N-substituted or N,N-disubstituted amino group (—NHR⁴⁴⁰; —NR⁴⁴¹R⁴⁴²); and
which, independently of each other, can be substituted with one or more substituents, which, can be the same or different; and,
wherein optionally, the pairs R⁴²⁶/R⁴²⁷, R⁴³⁰/R⁴³¹, R⁴³⁴/R⁴³⁵, R⁴³⁶/R⁴³⁷ and R⁴⁴I/R⁴⁴² independenly of each other, may form a part of a ring; and
wherein the substituents R⁴²⁰, R⁴²¹, R⁴²², R⁴²³, R⁴²⁴, R⁴²⁵, R⁴²⁶, R⁴²⁷, R⁴²⁸, R⁴²⁹, R⁴³⁰, R⁴³¹, R⁴³², R⁴³³, R⁴³⁴, R⁴³⁵, R⁴³⁶, R⁴³⁷, R⁴³⁸, R⁴³⁹, R⁴⁴⁰, R⁴⁴¹, and R⁴⁴², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (VII) 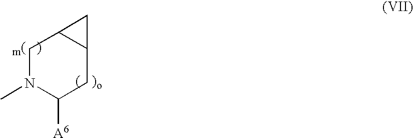
wherein m is equal to 1 or 2, and o is equal to 1 or 2, and m or o can be 0;
wherein A⁶ is a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁴⁶⁰), a boronic acid group (—B(OH)₂), a cyano group (—C≡N), a carboxylic acid group (—COOH), a carboxylic acid ester group (—COOR⁴⁶¹), a carboxylic acid anhydride group (—CO—O—CO—R⁴⁶²), a hydroxamic acid group (—CO—NH(OH)), a N-substituted hydroxamic acid group (—CO—NR⁴⁶ (OH)), a O-substituted hydroxamic acid group (—CO—NH(OR⁴⁶⁴)), a carboxamide group (—CO—NH₂), a N-substituted or N,N-disubstituted carboxylic acid amide group, (—CO—NHR⁴⁶⁵; —CO—NR⁴⁶⁶R⁴⁶⁷), an amido group (—HN—CO—R⁴⁶⁸), a sulfonic acid group (—SO₃H), a sulfonamide group (—SO₂—NH₂), a N-substituted or N,N-disubstituted sulfonamide group (—SO₂—NHR⁴⁶⁹; —SO₂—NR⁴⁷⁰R⁴⁷¹), an amidosulfone group (—NH—SO₂—R¹²), a sulfone group (—SO₂—R⁴⁷³), a phosphoric acid group (—OP(═O)(OH)₂), a phosphoric acid ester group (—OP(═O)(OR⁴⁷⁴)(oR⁴⁷⁵)), a phosphonic acid group (—P(═O)(OH)₂), an phosphonic acid ester group (—P(═O)(OR⁴⁷⁶)(OR⁴⁷⁷)), a halogen atom, a trifluormethyl group (—CF₃), a thiol group (—SH); a thioether group (—S—R⁴⁷⁸), a hydroxy group (—OH); an alkoxy group (—O—R⁴⁷⁹), a tetrazole group, an amino group (—NH₂), or a N-substituted or N,N-disubstituted amino group (—NHR⁴⁸⁰; —NR⁴⁸¹R⁴⁸²);
which, independently of each other, can be substituted with one or more substituents, which can be the same or different; and,
wherein optionally, the pairs R⁴⁶⁶/R⁴⁶⁷, R⁴⁷⁰/R⁴⁷¹, R⁴⁷⁴/R⁴⁷⁵, R⁴⁷⁶/R⁴⁷⁷ and R⁴⁸¹/R⁴⁸² independenly of each other, may form a part of a ring; and
wherein the substituents R⁴⁶⁰, R⁴⁶¹, R⁴⁶², R⁴⁶³, R⁴⁶⁴, R⁴⁶⁵, R⁴⁶⁶, R⁴⁶⁷, R⁴⁶⁸, R⁴⁶⁹, R⁴⁷⁰, R⁴⁷¹, R⁴⁷², R⁴⁷³, R⁴⁷⁴, R⁴⁷⁵, R⁴⁷⁶, R⁴⁷⁷, R⁴⁷⁸, R⁴⁷⁹, R⁴⁸⁰, R⁴⁸¹, and R⁴⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (VIII) 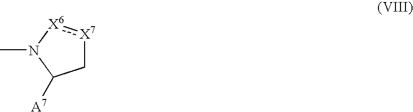
wherein X⁶ is selected from CR⁴⁹⁰R⁴⁹¹, O, S or NR⁴⁹², when the bond between X⁶ and X⁷ is a single bond; and
wherein X⁷ is selected from CR⁴⁹³R⁴⁹⁴, O, S, or NR⁴⁹⁵, when the bond between X⁶ and X⁷ is a single bond;
or alternatively,
wherein X⁶ is selected from CR⁴⁹⁶ or N, when the bond between X⁶ and X⁷ is a double bond; and
wherein X⁷ is selected from CR⁴⁹⁷ or N, when the bond between X⁶ and X⁷ is a double bond; and
wherein R⁴⁹⁰, R⁴⁹¹, R⁴⁹², R⁴⁹³, R⁴⁹⁴, R⁴⁹⁵, R⁴⁹⁶, and R⁴⁹⁷, independently of each other, are a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁵⁰⁰), a boronic acid group (—B(OH)₂), a cyano group (—C≡N), a carboxylic acid group (—COOH), a carboxylic acid ester group (—COOR⁵⁰¹), a carboxylic acid anhydride group (—CO—O—CO—R⁵⁰²), a hydroxamic acid group (—CO—NH(OH)), a N-substituted hydroxamic acid group (—CO—NR⁵⁰³(OH)), a O-substituted hydroxamic acid group (—CO—NH(OR⁵⁰⁴)), a carboxamide group (—CO—NH₂), a N-substituted or N,N-disubstituted carboxylic acid amide group, (—CO—NHR⁵⁰⁵; —CO—NR⁵⁰⁶R⁵⁰⁷), an amido group (—HN—CO—R⁵⁰⁸), a sulfonic acid group (—SO₃H), a sulfonamide group (—SO₂—NH₂), a N-substituted or N,N-disubstituted sulfonamide group (—SO₂—NHR⁵⁰⁹; —SO₂—NR⁵¹⁰R⁵¹¹), an amidosulfone group (—NH—SO₂—R⁵¹²), a sulfone group (—SO₂—R⁵¹³), a phosphoric acid group (—OP(═O)(OH)₂), a phosphoric acid ester group (—OP(═O)(OR⁵¹⁴)(OR⁵¹⁵)), a phosphonic acid group (—P(═O)(OH)₂), an phosphonic acid ester group (—P(═O)(OR⁵¹⁶)(OR⁵¹⁷)), a halogen atom, a trifluormethyl group (—CF₃), a thiol group (—SH); a thioether group (—S—R⁵¹⁸), a hydroxy group (—OH); an alkoxy group (—O—R⁵¹⁹), a tetrazole group, an amino group (—NH₂), or a N-substituted or N,N-disubstituted amino group (—NHR⁵²⁰; —NR⁵²R⁵²²); and
which, independently of each other, can be substituted with one or more substituents, which can be the same or different; and,
wherein optionally, any two the groups R⁴⁹⁰, R⁴⁹¹, R⁴⁹², R⁴⁹³, R⁴⁹⁴, R⁴⁹⁵, R⁴⁹⁶, and R⁴⁹⁷, if present, as well as the pairs R⁵⁰⁶/R⁵⁰⁷, R⁵¹⁰/R⁵¹¹, R⁵¹⁴/R⁵¹⁵, R⁵¹⁶/R⁵¹⁷ and R⁵²¹/R⁵²² independenly of each other, may form a part of a ring; and
wherein the substituents R⁵⁰⁰, R⁵⁰¹, R⁵⁰², R⁵⁰³, R⁵⁰⁴, R⁵⁰⁵, R⁵⁰⁶, R⁵⁰⁷, R⁵⁰⁸, R⁵⁰⁹, R⁵¹⁰, R⁵¹¹ R⁵¹², R⁵¹³, R⁵¹⁴, R⁵¹⁵, R⁵¹⁶, R⁵¹⁷, R⁵¹⁸, R⁵¹⁹, R⁵²⁰, R⁵²¹, and R⁵²², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group; and
wherein A⁷ is
a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁵⁴⁰), a boronic acid group (—B(OH)₂), a cyano group (—C≡N), a carboxylic acid group (—COOH), a carboxylic acid ester group (—COOR⁵⁴¹), a carboxylic acid anhydride group (—CO—O—CO—R⁵⁴²), a hydroxamic acid group (—CO—NH(OH)), a N-substituted hydroxamic acid group (—CO—NR⁵⁴³(OH)), a O-substituted hydroxamic acid group (—CO—NH(OR⁵)), a carboxamide group (—CO—NH₂), a N-substituted or N,N-disubstituted carboxylic acid amide group, (—CO—NHR⁵⁴⁵; —CO—NR⁵⁴R⁵⁴⁷), an amido group (—HN—CO—R⁵⁴⁸), a sulfonic acid group (—SO₃H), a sulfonamide group (—SO₂—NH₂), a N-substituted or N,N-disubstituted sulfonamide group (—SO₂—NHR⁵⁴⁹; —SO₂—NR⁵⁵⁰R⁵⁵¹), an amidosulfone group (—NH—SO₂—R⁵⁵²), a sulfone group (—SO₂—R⁵³), a phosphoric acid group (—OP(═O)(OH)₂), a phosphoric acid ester group (—OP(═O)(OR⁵⁵⁴)(OR⁵⁵⁵)), a phosphonic acid group (—P(═O)(OH)₂), an phosphonic acid ester group (—P(═O)(OR⁵⁶)(OR⁵⁵⁷)), a halogen atom, a trifluormethyl group (—CF₃), a thiol group (—SH); a thioether group (—S—R⁵⁵⁸), a hydroxy group (—OH); an alkoxy group (—O—R⁵⁵⁹), a tetrazole group, an amino group (—NH₂), or a N-substituted or N,N-disubstituted amino group (—NHR⁵⁶⁰; —NR⁵⁶R⁵⁶²); and
which, independently of each other, can be substituted with one or more substituents, which can be the same or different; and,
wherein optionally, the pairs R⁵⁴⁶/R⁵⁴⁷, R⁵⁵⁰/R⁵⁵¹, R⁵⁵⁴/R⁵⁵⁵, R⁵⁵⁶/R⁵⁵⁷ and R⁵⁶¹/R⁵⁶², independenly of each other, may form a part of a ring; and
wherein the substituents R⁵⁴⁰, R⁵⁴¹, R⁵⁴², R⁵⁴³, R⁵⁴⁴, R⁵⁴⁵, R⁵⁴⁶, R⁵⁴⁷, R⁵⁴⁸, R⁵⁴⁹, R⁵⁵⁰, R⁵⁵¹, R⁵⁵², R⁵⁵³, R⁵⁵⁴, R⁵⁵⁵, R⁵⁵⁶, R⁵⁵⁷, R⁵⁸⁸, R⁵⁵⁹, R⁵⁶⁰, R⁵⁶¹, and R⁵⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (IX) or (IXa) 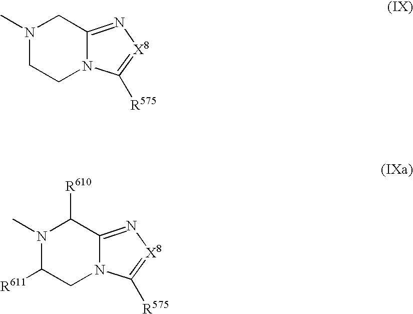
wherein x⁸ is N or CR⁵⁷⁰; and
wherein R⁵⁷⁰, R⁵⁷¹, R⁶¹⁰ and R⁶¹¹ independently of each other, are a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R⁵⁸⁰), a boronic acid group (—B(OH)₂), a cyano group (—C≡N), a carboxylic acid group (—COOH), a carboxylic acid ester group (—COOR⁵⁸¹), a carboxylic acid anhydride group (—CO—O—CO—R⁵⁸²), a hydroxamic acid group (—CO—NH(OH)), a N-substituted hydroxainic acid group (—CO—NR⁵⁸³(OH)), a O-substituted hydroxamic acid group (—CO—NH(OR⁵⁸⁴)), a carboxamide group (—CO—NH₂), a N-substituted or N,N-disubstituted carboxylic acid amide group, (—CO—NHR⁵⁸⁵; —CO—NR⁵⁸⁶, R⁵⁸⁷), an amido group (—HN—CO—R⁵⁸⁸), a sulfonic acid group (—SO₃H), a sulfonamide group (—SO₂—NH₂), a N-substituted or N,N-disubstituted sulfonamide group (—SO₂—NHR⁵⁸⁹; —SO₂—NR⁵⁹OR⁵⁹¹), an amidosulfone group (—NH—SO₂—R⁵⁹²), a sulfone group (—SO₂—R⁵⁹³), a phosphoric acid group (—OP(═O)(OH)₂), a phosphoric acid ester group (—OP(═O)(OR⁵⁹⁴)(OR⁵⁹⁵)), a phosphonic acid group (—P(═O)(OH)₂), an phosphonic acid ester group (—P(═O)(OR⁵⁹⁶)(OR⁵⁹⁷)), a halogen atom, a trifluormethyl group (—CF₃), a thiol group (—SH); a thioether group (—S—R⁵⁹⁸), a hydroxy group (—OH); an alkoxy group (—O—R⁵⁹⁹), a tetrazole group, an amino group (—NH₂), or a N-substituted or N,N-disubstituted amino group (—N600; —NR⁶⁰¹R⁶⁰²);
which, independently of each other, can be substituted with one or more substituents, which can be the same or different; and,
wherein optionally, the pairs R¹⁷⁰/R¹¹, if present, as well as the pairs R⁵⁸⁶/R⁵⁹⁷, R⁵⁹⁰/R⁵⁹¹, R⁵⁹⁴/R⁵⁹⁵, R⁵⁹⁶/R⁵⁹⁷ and R⁶⁰¹/R⁶⁰², independenly of each other, may form a part of a ring; and
wherein the substituents R⁵⁸⁰, R⁵⁸¹, R⁵⁸², R⁵⁸³, R⁵⁸⁴, R⁵⁸⁵, R⁵⁸⁶, R⁵⁸⁷, R⁵⁸⁸, R⁵⁸⁹, R⁵⁹⁰, R⁵⁹¹, R⁵⁹², R⁵⁹³, R⁵⁹⁴, R⁵⁹⁵, R⁵⁹⁶, R⁵⁹⁷, R⁵⁹⁸, R⁵⁹⁹, R⁶⁰⁰, R⁶⁰¹, and R⁶⁰², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (X) 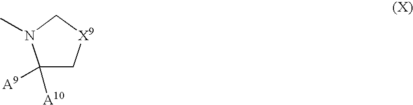
wherein the groups X⁹ is CR⁹⁰⁰R⁹⁰¹, S, SO, SO₂or NR⁹⁰²
wherein R⁹⁰⁰, R⁹⁰¹ and R⁹⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR⁹¹⁰R⁹¹¹.
wherein A⁹ and A¹⁰ are, independently of each other, selected from hydrogen, cyano, —C(═O)NR⁹¹², R⁹¹³, or C₁, C₂, C₃, C₄, C₅or C₆alkyl, which is linear or branched and is optionally substituted with 1, 2, 3, 4, or 5 halogens;
wherein
R⁹¹⁰ and R⁹¹², are, independently of each other, selected from hydrogen, or C₁, C₂, C₃, C₄, C₅or C₆alkyl, which is linear or branched and is optionally substituted with 1, 2, 3, 4, or 5 halogens; and
R⁹¹¹ and R⁹¹³, are, independently of each other, selected from the group consisting of
(1) phenyl, which is optionally substituted with 1, 2, 3, 4, or 5, substituents independently selected from halogen and R⁹²⁰;
(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of
(a) hydroxy,
(b) —COOH,
(c) —COO(C₁, C₂, C₃, C₄, C₅or C₆alkyl), i.e. ester,
(d) phenyl,
(e) naphthyl,
(f) C₃, C₄, C₅or C₆cycloalkyl,
(g) a 5- or 6 membered heterocycle which may be saturated or unsaturated comprising 1, 2, 3, or 4 heteroatoms independently selected from nitrogen , oxygen or sulfur;
(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;
wherein said C₃, C₄, C₅or C₆cycloalkyl, phenyl, naphthyl, are optionally substituted with 1, 2, 3, 4, or 5 substituents independently selected from halogen and R⁹²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R⁹²⁰; and
(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, CS or C₆ alkyl, and —OC₁, —OC₂, —OC₃, —OC₄, —OC₅or —OC₆alkyl being linear or branched and optionally substituted with 1, 2, 3, 4, or 5 halogens;
wherein R⁹²⁰ is selected from the group consisting of:
(1) hydroxy;
(2) cyano;
(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;
(4) C₁, C₂, C₃, C₄, C₅, C₆, C₇, C₈, C₉ or C₁₀ alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from
(a) hydroxy;
(b) —COOH;
(c) —COO(C₁, C₂, C₃, C₄, C₅or C₆alkyl) i.e. ester, which may linear or branched and is optionally substituted with 1, 2, 3, 4, or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(f) —CONR⁹²⁵, R⁹²⁵;
(g) —SO₂NR⁹²⁵R⁹²⁵;
(h) —NR⁹²⁵—C(═O)R⁹²⁵
(i) —NR⁹²⁵C(═O)NR⁹²¹R^{925;}
(j) —NR⁹²⁵COOR⁹³⁰
(k) —O—CO—R⁹³⁰
(l) —O—CO—NR⁹²⁵R⁹²⁵;
(m) —NR⁹²⁵SO₂R⁹³⁰;
(n) _NR⁹²⁵R⁹²⁵;
(o) phenyl which is optionally substituted with 1, 2, 3, 4, or 5 group independently selected from halogen, hydroxy, C₁, C₂, C₃, C₄, Cs or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and
(p) C₃, C₄C₅or C₆cycloalkyl, which is optionally substituted with 1, 2, 3, 4, 5, or 6 halogens;
(5) OC₁, OC₂, OC₃, OC₄, OC₅, OC₆, OC₇, OC₈, OC₉or OC₁₀alkyl, which is linear or branched and is optionally substituted with 0, 1, 2, 3, 4, or 5 halogen atoms and 0, 1, or 2 substitutents selected from
(a) hydroxy;
(b) —COOH;
(c) —COO(C₁, C₂, C₃, C₄, C₅or C₆alkyl) i.e. ester, which may be linear or branched and is optionally substituted with 1, 2, 3, 4 or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OCs or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, Cs or C₆alkyl, and —OC₁, —OC₂, —OC₃, —OC₄, —OC₅or —OC₆alkyl, said C₁, C₂, C₃, C₄, C₅ or C₆alkyl, and —OC₁, —OC₂, —OC₃, —OC₄, —OC₅or —OC₆alkyl being linear or branched and optionally substituted with 1, 2, 3, 4, or 5 halogens;
(f) —CONR⁹²⁵, R⁹²⁵;
(g) —SO₂NR⁹²⁵, R⁹²;
(h) —NR⁹²⁵—C(═O)R¹⁹²
(i) —NR⁹²⁵—C(═O)NR⁹²⁵R⁹²⁵;
(j) —NR⁹²⁵ COOR⁹³⁰
(k) —O—CO—R⁹³⁰
(l) —O—CO—NR⁹²⁵, R⁹²;
(m) —NR⁹²⁵SO₂R⁹³⁰;
(n) NR⁹²⁵, R⁹²⁵;
(o) phenyl, which is optionally substituted with 1, 2, 3, 4, or 5 groups independently selected from halogen, hydroxy, C₁, C₂, C₃, C₄, CS or C₆alkyl, —OC₁, —OC₂, —OC₃, —OC₄, —OC₅or —OC₆alkyl, —COOH, —COO(C₁, C₂, C₃, C₄, Cs or C₆alkyl) i.e. ester, said C₁, C₂, C₃, C₄, CS or C₆alkyl, —OC₁, —OC₂, —OC₃, —OC₄, —OC₅or —OC₆alkyl, —COOH, —COO(C₁, C₂, C₃, C₄, CS or C₆alkyl) i.e. ester being linear or branched and optionally substituted with 1, 2, 3, 4, 5, or 6 substitutents independently selected from 0 or 1 C₃, C₄CS or C₆cycloalkyl and 0, 1, 2, 3, 4, or 5 halogens, and
(p) C₃, C₄CS or C₆cycloalkyl, which is optionally substituted with 1, 2, 3, 4, 5, or 6 halogens;
(6) —COOH;
(7) —COO(C₁, C₂, C₃, C₄, C₅ or C₆alkyl) i.e. ester, which may be linear or branched and is optionally substituted with 1, 2, 3, 4, 5 halogens;
(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.
(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(10)—CONR⁹²⁵R⁹²⁵;
(11) —SO₂NR⁹²⁵R⁹²⁵;
(12) —NR²¹—C(═O)R⁹²¹
(13)—NR⁹²⁵—C(═O)NR⁹²⁵R⁹²⁵;
(14) —NR⁹²⁵COOR⁹³⁰
(15)—O—CO—R⁹³⁰
(16)—O—CO_NR⁹²⁵R⁹²⁵;
(17) —NR⁹²⁵SO₂R⁹³⁰;
(18) —NR⁹²⁵R⁹²⁵;
(19) phenyl , which is optionally substituted with 1, 2, 3, 4, or 5 group independently selected from halogen, hydroxy, C₁, C₂, C₃, C₄, Cs or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;
wherein R⁹³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.
wherein R⁹²⁵ is selected from R⁹³⁰ and hydrogen.
wherein the group PM
has the formula (XI) 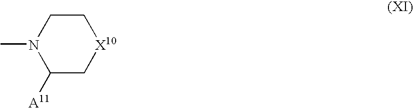
wherein the groups X¹⁰ is CR¹⁰⁰⁰R¹⁰⁰¹, S, SO, SO₂or NR¹⁰⁰²
wherein R¹⁰⁰⁰, R¹⁰⁰¹ and R¹⁰⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR⁹¹⁰R⁹¹¹.
and A¹¹ is selected from
hydrogen, cyano, —C(═O)NR¹⁰¹²R¹⁰¹³ or C₁, C₂, C₃, C₄, C₅or C₆alkyl, which is linear or branched and is optionally substituted with 1, 2, 3, 4, or 5 halogens;
wherein
R¹⁰¹⁰ and R¹⁰¹², are, independently of each other, selected from hydrogen, or C₁, C₂, C₃, C₄, C₅or C₆alkyl, which is linear or branched and is optionally substituted with 1, 2, 3, 4, or 5 halogens; and
R¹⁰¹¹ and R¹⁰¹³, are, independently of each other, selected from the group consisting of (1) phenyl, which is optionally substituted with 1, 2, 3, 4, or 5, substituents independently selected from halogen and R¹⁰²⁰;
(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of
(a) hydroxy,
(b) —COOH,
(c) —COO(C₁, C₂, C₃, C₄, C₅or C₆alkyl), i.e. ester,
(d) phenyl,
(e) naphthyl,
(f) C₃, C₄, C₅or C₆cycloalkyl,
(g) a 5- or 6 membered htereocycle which may be saturated or unsaturated comprising 1, 2, 3, or 4 heteroatoms independently selected from nitrogen , oxygen or sulfur;
(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;
wherein said C₃, C₄, C₅or C₆cycloalkyl, phenyl, naphthyl, are optionally substituted with 1, 2, 3, 4, or 5 substituents independently selected from halogen and R⁰²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R¹⁰²⁰; and
(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;
wherein R¹⁰²⁰ is selected from the group consisting of:
(1) hydroxy;
(2) cyano;
(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;
(4) C₁, C₂, C₃, C₄, Cs, C₆, C₇, C₈, C₉or Clo alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from
(a) hydroxy;
(b) —COOH;
(c) —COO(C₁, C₂, C₃, C₄, C₅or C₆alkyl) i.e. ester, which may linear or branched and is optionally substituted with 1, 2, 3, 4, or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, Cs or C₆alkyl, and —OC₁, —OC₂, —OC₃, —OC₄, —OC₅or —OC₆alkyl, said C₁, C₂, C₃, C₄, Cs or C₆alkyl, and —OC₁, —OC₂, —OC₃, —OC₄, —OC₅or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, Cs or C₆alkyl, and —OC₁, —OC₂, —OC₃, —OC₄, —OC₅or —OC₆alkyl, said C₁, C₂, C₃, C₄, Cs or C₆alkyl, and —OC₁, —OC₂, —OC₃, —OC₄, —OC₅or —OC₆alkyl being linear or branched and optionally substituted with 1, 2, 3, 4, or 5 halogens;
(e) —CONR¹⁰²⁵R¹⁰²⁵;
(g) —SO₂NR¹⁰²⁵R¹⁰²⁵;
(h) —NR¹⁰²⁵—C(═O)R¹⁰²⁵
(i) —NR¹⁰²⁵—C(═O)NR¹⁰²⁵, R¹⁰²⁵;
(j) —NR¹⁰²⁵COOR¹⁰³⁰
(k) —O—CO—R¹⁰³⁰
(l)—O—CO—NR¹⁰²⁵R¹⁰²⁵;
(m)—NR¹⁰²⁵SO₂R¹⁰³⁰
(n) —NR¹⁰²⁵R¹⁰²⁵;
(o) phenyl which is optionally substituted with 1, 2, 3, 4, or 5 group independently selected from halogen, hydroxy, C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅ or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and
(p) C₃, C₄C₅or C₆cycloalkyl, which is optionally substituted with 1, 2, 3, 4, 5, or 6 halogens;
(5) OC₁, OC₂, OC₃, OC₄, OC₅, OC₆, OC₇, OC₈, OC₉or OC₁₀alkyl, which is linear or branched and is optionally substituted with 0, 1, 2, 3, 4, or 5 halogen atoms and 0, 1, or 2 substitutents selected from
(a) hydroxy;
(b) —COOH;
(c) —COO(C₁, C₂, C₃, C₄, C₅or C₆alkyl) i.e. ester, which may be linear or branched and is optionally substituted with 1, 2, 3, 4 or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(f) —CONR¹⁰²⁵R¹⁰²⁵;
(g) —SO₂NR¹⁰²⁵R¹⁰²⁵;
(h) —NR¹⁰²⁵—C(═O)R¹⁰²⁵
(i) —NR¹⁰²⁵—C(═O)NR¹⁰²⁵, R¹⁰²⁵
(j)—NR¹⁰²⁵COOR¹⁰³⁰
(k) —O—CO—R¹⁰³⁰
(l)—O—CO—NR¹⁰²⁵, R¹⁰²⁵;
(m) —NR¹⁰²⁵, R¹⁰³⁰;
(n) —NR¹⁰²⁵R¹⁰²⁵;
(o) phenyl, which is optionally substituted with 1, 2, 3, 4, or 5 groups independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and
(p) C₃, C₄C₅or C₆cycloalkyl, which is optionally substituted with 1, 2, 3, 4, 5, or 6 halogens;
(6) —COOH;
(7) —COO(C₁, C₂, C₃, C₄, C₅or C₆alkyl) i.e. ester, which may be linear or branched and is optionally substituted with 1, 2, 3, 4, 5 halogens;
(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.
(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(10)—CONR¹⁰²⁵R¹⁰²⁵;
(11) —SO₂NR¹⁰²⁵, R¹⁰²⁵;
(12) —NR¹⁰²⁵—C(═O)R¹⁰²⁵
(13)—NR¹⁰²⁵—C(═O)NR¹⁰²⁵R¹⁰²⁵;
(14) —NR⁹²⁵COOR¹⁰³⁰
(15)—O—CO—R¹⁰³⁰
(16)—O—CO—NR¹¹²⁵, R²⁵;
(17)—NR¹⁰²⁵SO₂R¹⁰³⁰;
(18) —NR¹⁰²⁵R¹⁰²⁵, R¹⁰²⁵;
(19) phenyl , which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;
wherein R¹⁰³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.
wherein R¹⁰²⁵ is selected from R¹⁰³⁰ and hydrogen.
or wherein the group PM
has the formula (XII) 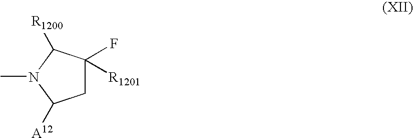
wherein the groups R¹²⁰¹ is hydrogen or fluoro.
wherein R¹²⁰⁰ und A¹² is selected from hydrogen and cyano, and the other is hydrogen.
or wherein the group PM
has the formula XIII: 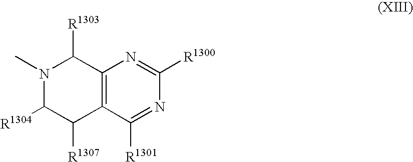
wherein:
R¹³⁰⁰ and R¹³⁰¹ are independently selected from the group consisting of:
(1) hydrogen,
(2) CN,
(3) C₁₋₁₀alkyl, which is linear or branched which is unsubstituted or substituted with:
a) halogen, or
b) phenyl, which is unsubstituted or substituted with 1-5 substitutents independently selected from halogen, CN, OH, R¹³⁰², OR¹³⁰², NHSO₂R¹³⁰², N(C₁₋₆ alkyl)SO₂R¹³⁰², SO₂R¹³⁰², SO₂NR³⁰⁵R¹³⁰⁶ NR¹¹³⁰⁵R¹³⁰⁶, CONR¹³⁰⁵R¹³⁰⁶ CO₂H, and CO₂C₁₋₆alkyl, wherein the C₁₋₆alkyl is linear or branched,
(4) phenyl which is unsubstituted or substituted with 1-5 substitutents independently selected from halogen, CN, OH, R¹³⁰², OR¹³⁰², NSO₂R¹³⁰² N(C₁₋₆alkyl)SO₂R¹³⁰², SO₂R¹³⁰² SONR¹³⁰⁵, R¹³⁰⁶, NR¹³⁰⁵R¹³⁰⁶, CONR¹³⁰⁵R¹³⁰⁶ CO₂H, and CO₂C₁₋₆alkyl, wherein the C₁₋₆alkyl is linear or branched,
(5) a 5- or 6-membered heterocyclic which may be saturated or unsaturated comprising 1-4 heteroatoms independently selected from N, S and O, the heterocycle being unsubstituted or substituted with 1-3 substituents independently selected from oxo, halogen, NO₂, CN, OH, R¹³⁰², OR¹³⁰², NHSO₂R¹³⁰², N(C₁₋₆alkyl)SO₂R¹³⁰² SO₂R¹³⁰²R¹³⁰² SO₂NR¹³⁰⁵, R¹³⁰⁶ NR¹³⁰⁵R¹³⁰⁶, CONR¹³⁰⁵, R¹³⁰⁶, CO₂H, and CO₂C₁₋₆alkyl, wherein the C₁₋₆alkyl is linear or branched,
(6) C₃₋₆cycloalkyl, which is optionally substituted with 1-5 substituents independently selected from halogen, OH, C₁₋₆alkyl, and OC₁₋₆alkyl, wherein the C₁₋₆alkyl and OC₁₋₆ alkyl are linear or branched and optionally substituted with 1-5 halogens,
(7) OH,
(8) OR¹³⁰² and
(9) NR¹³⁰⁵R¹³⁰⁶;
R¹³⁰² is C₁₋₆alkyl, which is linear or branched and which is unsubstituted or substituted with 1-5 groups independently selected from halogen, CO₂H, and CO₂C₁₋₆alkyl, wherein the C₁₋₆alkyl is linear or branched;
R¹³⁰³, R¹³⁰⁴ and R¹³⁰⁷ are independently selected from the group consisting of:
(1) hydrogen,
(2) C₁₋₁₀alkyl, which is linear or branched and which is unsubstituted or substituted with one or more substituted selected from:
a) halogen,
b) hydroxy,
c) phenyl, which is unsubstituted or substituted with 1-5 substituted independently selected from halogen, OH, C₁₋₆alkyl, and OC₁₋₆alkyl, wherein the C₁₋₆alkyl is linear or branched and optionally substituted with 1-5 halogens,
d) naphthyl, wherein the naphthyl is optionally substituted with 1-5 substituents independently selected from halogen, OH, C₁₋₆alkyl, and OC₁₋₆alkyl, wherein the C₁₋₆alkyl is linear or branched and optionally substituted with 1-5 halogens,
e) CO₂H,
f) CO₂C₁₋₆alkyl,
g) coNR¹³⁰⁵R¹³⁰⁶,
(3) CN,
(4) phenyl which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens,
(5) naphthyl which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens,
(6) CO₂H,
(7) CO₂C₁₋₆alkyl,
(8) CONR¹³⁰⁵, R³⁰⁶, and
(9) C₃₋₆cycloalkyl, which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens;
R¹³⁰⁵ and R¹³⁰⁶ are independently selelcted from the group consisting of:
(1) hydrogen,
(2) phenyl, which is unsubstituted or substituted with substituents independently selected from halogen, OH, C₁₋₆alkyl, and OC₁₋₆alkyl, wherein the C₁₋₆alkyl is linear or branched and optionally substituted with 1-5 halogens,
(3) C₃₋₆cycloalkyl, which is unsubstituted or substituted with 1-5 substituents independently selected from C₁₋₆alkyl, and OC₁₋₆alkyl, wherein the C₁₋₆alkyl is linear or branched and optionally substituted with 1-5 halogens,
(4) C₁₋₆alkyl, which is linear or branched and which is unsubstituted or substituted with:
a) halogen, or
b) phenyl, which is unsubstituted or substituted with 1-5 substituents independently selected from halogen, OH, C₁₋₆alkyl, and OC₁₋₆alkyl, wherein the C₁₋₆alkyl is linear or branched and optionally substituted with 1-5 halogens,
or wherein R¹³⁰⁵ and R¹³⁰⁶ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆alkyl, and C₁₋₆alkoxy, wherein alkyl and alkoxy are unsubstituted with one to five halogens;
or wherein the group PM
has the formula XIV: 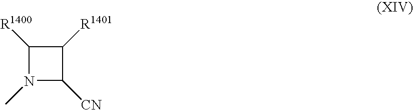
wherein R¹⁴⁰⁰ and R¹⁴⁰¹, independently of each other, are
a hydrogen atom (—H); or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R¹⁴⁰²), a boronic acid group (—B(OH)₂), a cyano group (—C≡N), a carboxylic acid group (—COOH), a carboxylic acid ester group (—COOR¹⁴⁰³), a carboxylic acid anhydride group (—CO—O—CO—R¹⁴⁰⁴), a hydroxamic acid group (—CO—NH(OH)), a N-substituted hydroxamic acid group (—CO—NR¹⁴⁰⁵(OH)), a O-substituted hydroxamic acid group (—CO—NH(OR¹⁶)), a carboxamide group (—CO—NH₂), a N-substituted or N,N-disubstituted carboxylic acid amide group, (—CO—NHR¹⁴⁰⁷; —CO—NR¹⁴⁰⁸R¹⁴⁰⁹), an amido group (—HN—CO—R¹⁴¹⁰), a sulfonic acid group (—SO₃H), a sulfonamide group (—SO₂—NH₂), a N-substituted or N,N-disubstituted sulfonamide group (—SO₂—NHR¹⁴¹¹; —SO₂—NR¹⁴¹²R¹⁴¹³), an amidosulfone group (—NH—SO₂—R¹⁴¹⁴), a sulfone group (—SO₂—R¹⁴¹⁵), a phosphoric acid group (—OP(═O)(OH)₂), a phosphoric acid ester group (—OP(═O)(OR¹⁴¹⁶)(OR¹⁴¹⁷)), a phosphonic acid group (—P(═O)(OH)₂), an phosphonic acid ester group (—P(═O)(OR¹⁴¹⁸)(OR¹⁴¹⁹)), a halogen atom, a trifluormethyl group (—CF₃), a thiol group (—SH); a thioether group (—S—R¹⁴²⁰), a hydroxy group (—OH); an alkoxy group (—O—R¹⁴²¹), a tetrazole group, an amino group (—NH₂), or a N-substituted or N,N-disubstituted amino group (—NHR¹⁴²²; —NR¹⁴²³R¹⁴²⁴); and
which, independently of each other, can be substituted with one or more substituents, which can be the same or different; and,
wherein optionally, the pairs R¹⁴⁰⁸/R¹⁴⁰⁹, R¹⁴¹²/R¹⁴¹³, R¹⁴¹⁶/R¹⁴¹⁷, R¹⁴¹⁸/R¹⁴¹⁹ and R¹⁴²³/R¹⁴²⁴, independenly of each other, may form a part of a ring; and
wherein the substituents R¹⁴⁰², R¹⁴⁰³, R¹⁴⁰⁴, R¹⁴⁰⁵, R¹⁴⁰⁶, R¹⁴⁰⁷, R¹⁴⁰⁸, R¹⁴⁰⁹, R¹⁴¹⁰, R^{1411 R}¹⁴¹², R¹⁴¹³, R¹⁴¹⁴, R¹⁴¹⁵, R¹⁴¹⁶, R¹⁴¹⁷, R¹⁴¹⁸, R¹⁴¹⁹, R¹⁴²⁰, R¹⁴²¹, and R¹⁴²², R¹⁴²³, and R¹⁴²⁴, independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula XV: 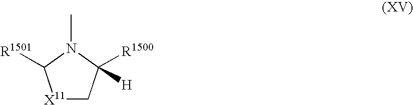
wherein X¹¹is CH₂, CHF or CF₂;
wherein R¹⁵⁰⁰ is selected from the group consisting of alkylcarbonyl, arylcarbonyl, cyano, heterocyclecarbonyl, R¹⁵⁰²R¹⁵⁰³NC(O)—, B(OR¹⁵⁰⁴)₂, (1,2,3)-dioxoborolane and 4,4,5,5-tetramethyl(1 ,2,3)-dioxoborolane;
wherein R¹⁵⁰¹ is selected from the group consisting of alkoxyalkyl, alkyl, alkylcarbonyl, alkenyl, alkynyl, allenyl, arylalkyl, cycloalkyl, cycloalkylalkyl, cyano, haloalkyl, haloalkenyl, heterocyclealkyl, and hydroxyalkyl;
wherein R¹⁵⁰², R¹⁵⁰³ and R¹⁵⁰⁴ are each independently selected from the group consisting of hydrogen, alkyl, and arylalkyl;
with the proviso that the following compounds are excluded:
glutamin-thiazolidin (=Gln-Thia), glutamin-pyrrolidin (=Gln-Pyrr) (from WO 03/072556), glutamin-pyrrolidin-2-carboxylic acid (=Gln-Pro), glutamin-pyrrolidin-2-carboxamid (=Gln-Pro amid), and (S,S) 4-Amino-5-(2-cyano-2,5-dihydro-pyrrol-1-yl)-6-oxo-pentanoic acid amide (Gln -2-cyano-2,5-dihydro-pyrrolidin) (from WO 01/55105).

2. The compound according to claim 1
wherein n is 0 or 1;
wherein R¹, R², R³, R⁴, R⁵, R⁶, R⁷, R⁸, R⁹, R¹⁰, and R¹¹ independently of each other are
a hydrogen atom; or
a substituted or unsubstituted alkyl group having 1 to 30 carbon atoms; or
a substituted or unsubstituted alkenyl group having 2 to 30 carbon atoms; or
a substituted or unsubstituted alkinyl group having 2 to 30 carbon atoms; or
a substituted or unsubstituted cycloalkyl group having 3 to 30 carbon atoms; or
a substituted or unsubstituted cycloalkenyl group having 3 to 30 carbon atoms;
or a substituted or unsubstituted cycloalkinyl group having 6 to 30 carbon atoms; or
a substituted or unsubstituted heteroalkyl group having 1 to 30 carbon atoms and 1 to 6 hetero atoms each independently selected from oxygen, nitrogen or sulfur; or
a substituted or unsubstituted heteroalkenyl group having 2 to 30 carbon atoms and 1 to 6 hetero atoms each independently selected from oxygen, nitrogen or sulfur; or
a substituted or unsubstituted heteroalkinyl group having 2 to 30 carbon atoms and 1 to 6 hetero atoms each independently selected from oxygen, nitrogen or sulfur; or
a substituted or unsubstituted heterocycloalkyl group having 1 to 30 carbon atoms, and 1 to 6 hetero atoms each independently selected from oxygen, nitrogen or sulfur; or
a substituted or unsubstituted heterocycloalkenyl group having 2 to 30 carbon atoms, and 1 to 6 hetero atoms each independently selected from oxygen, nitrogen or sulfur; or
a substituted or unsubstituted aryl group having 3 to 30 carbon atoms; or
a substituted or unsubstituted heteroaryl group having 1 to 30 carbon atoms, and 1 to 10 hetero atoms, each independently selected from oxygen, nitrogen or sulfur; or
a substituted or unsubstituted aryl-alkyl group having at least one substituted or unsubstituted aryl group each having 1 to 30 carbon atoms, and at least one substituted or unsubstituted alkyl group each having 1 to 30 carbon atoms; or
a substituted or unsubstituted heteroaryl-alkyl group having at least one substituted or unsubstituted heteroaryl group each having 1 to 30 carbon atoms, and 1 to 10 hetero atoms, each independently selected from oxygen, nitrogen or sulfur, and further, at least one substituted or unsubstituted alkyl group having having 1 to 30 carbon atoms; or
a substituted or unsubstituted aryl-heteroalkyl group having at least one substituted or unsubstituted aryl group each having 3 to 30 carbon atoms, and at least one substituted or unsubstituted heteroalkyl group each having 1 to 30 carbon atoms and 1 to 6 hetero atoms each independently selected from oxygen, nitrogen or sulfur; or
a substituted or unsubstituted heteroaryl-heteroalkyl group having at least one substituted or unsubstituted heteroaryl group each having 1 to 30 carbon atoms, and 1 to 10 hetero atoms, each independently selected from oxygen, nitrogen or sulfur, and further, at least one substituted or unsubstituted heteroalkyl group each having 1 to 30 carbon atoms and 1 to 6 hetero atoms each independently selected from oxygen, nitrogen or sulfur; or
a carbaldehyde (—CHO), a ketone group (—CO—R²⁰), a boronic acid group (—B(OH)₂), a cyano group (—C≡N), a carboxylic acid group (—COOH), a carboxylic acid ester group (—COOR²¹), a carboxylic acid anhydride group (—CO—O—CO—R²²), a hydroxamic acid group (—CO—NH(OH)), a N-substituted hydroxamic acid group (—CO—NR²³(OH)), a O-substituted hydroxamic acid group (—CO—NH(OR²⁴)), a carboxamide group (—CO—NH₂), a N-substituted or N,N-disubstituted carboxylic acid amide group, (—CO—NHR²⁵; —CO—NR²⁶R²⁷), an amido group (—HN—CO—R²⁸), a sulfonic acid group (—SO₃H), a sulfonamide group (—SO₂—NH₂), a N-substituted or N,N-disubstituted sulfonamide group (—SO₂—NHR²⁹; —SO₂—NR³⁰R³¹), an amidosulfone group (—NH—SO₂—R³²), a sulfone group (—SO₂—R³³), a phosphoric acid group (—OP(═O)(OH)₂), a phosphoric acid ester group (—OP(═O)(OR³⁴)(OR³⁵)), a phosphonic acid group (—P(═O)(OH)₂), an phosphonic acid ester group (—P(═O)(OR³⁶)(OR³⁷)), a halogen atom, a trifluormethyl group (—CF₃), a thiol group (—SH); a thioether group (—S—R³⁸), a hydroxy group (—OH); an alkoxy group (—O—R³⁹), a tetrazole group, an amino group (—NH₂), or a N-substituted or N,N-disubstituted amino group (—NHR⁴⁰; —NR⁴¹, R⁴²);
which each independently can be substituted with one or more substituents, which can be the same or different; and,
wherein optionally, any two of the groups R¹, R², R³, R⁴, R⁵, R⁶, R⁷, R⁸, R⁹, R¹⁰, and R¹¹, as well the pairs R²⁶/R²⁷, R³⁰/R³¹, R³⁴/R³⁵, R³⁶/R³⁷ and R⁴¹/R⁴², independenly of each other, may form a part of a ring; and
wherein the substituents R²⁰, R²¹, R²², R²³, R²⁴, R²⁵, R²⁶, R²⁷, R²⁸, R²⁹, R³⁰, R³¹, R³², R³³R³⁴, R³⁵, R³⁶, R³⁷, R³⁸, R³⁹, R⁴⁰, R⁴¹, and R⁴² independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group.

3. The compound according to claim 1
wherein n is 0 or 1;
wherein R¹, R², R³, R⁴, R⁵, R⁶, R⁷, R⁸, R⁹, R¹⁰, and R¹¹ independently of each other are
a hydrogen atom; or
a substituted or unsubstituted alkyl group having 1 to 20 carbon atoms; or
a substituted or unsubstituted alkenyl group having 2 to 20 carbon atoms; or
a substituted or unsubstituted alkinyl group having 2 to 20 carbon atoms; or
a substituted or unsubstituted cycloalkyl group having 3 to 20 carbon atoms; or
a substituted or unsubstituted cycloalkenyl group having 3 to 20 carbon atoms;
or a substituted or unsubstituted cycloalkinyl group having 6 to 20 carbon atoms; or
a substituted or unsubstituted heteroalkyl group having 1 to 20 carbon atoms and 1 to 3 hetero atoms each independently selected from oxygen, nitrogen or sulfur; or
a substituted or unsubstituted heteroalkenyl group having 2 to 20 carbon atoms and 1 to 3 hetero atoms each independently selected from oxygen, nitrogen or sulfur; or
a substituted or unsubstituted heteroalkinyl group having 2 to 20 carbon atoms and 1 to 3 hetero atoms each independently selected from oxygen, nitrogen or sulfur; or
a substituted or unsubstituted heterocycloalkyl group having 1 to 20 carbon atoms, and 1 to 3 hetero atoms each independently selected from oxygen, nitrogen or sulfur; or
a substituted or unsubstituted heterocycloalkenyl group having 2 to 20 carbon atoms, and 1 to 3 hetero atoms each independently selected from oxygen, nitrogen or sulfur; or
a substituted or unsubstituted aryl group having 3 to 20 carbon atoms; or
a substituted or unsubstituted heteroaryl group having 1 to 20 carbon atoms, and 1 to 4 hetero atoms, each independently selected from oxygen, nitrogen or sulfur; or
a substituted or unsubstituted aryl-alkyl group having at least one substituted or unsubstituted aryl group each having 1 to 20 carbon atoms, and at least one substituted or unsubstituted alkyl group each having 1 to 20 carbon atoms; or
a substituted or unsubstituted heteroaryl-alkyl group having at least one substituted or unsubstituted heteroaryl group each having 1 to 20 carbon atoms, and 1 to 4 hetero atoms, each independently selected from oxygen, nitrogen or sulfur, and further, at least one substituted or unsubstituted alkyl group having having 1 to 20 carbon atoms; or
a substituted or unsubstituted aryl-heteroalkyl group having at least one substituted or unsubstituted aryl group each having 3 to 20 carbon atoms, and at least one substituted or unsubstituted heteroalkyl group each having 1 to 20 carbon atoms and 1 to 3 hetero atoms each independently selected from oxygen, nitrogen or sulfur; or
a substituted or unsubstituted heteroaryl-heteroalkyl group having at least one substituted or unsubstituted heteroaryl group each having 1 to 20 carbon atoms, and 1 to 4 hetero atoms, each independently selected from oxygen, nitrogen or sulfur, and further, at least one substituted or unsubstituted heteroalkyl group each having 1 to 20 carbon atoms and 1 to 4 hetero atoms each independently selected from oxygen, nitrogen or sulfur; or
a carbaldehyde (—CHO), a ketone group (—CO—R²⁰), a boronic acid group (—B(OH)₂), a cyano group (—C≡N), a carboxylic acid group (—COOH), a carboxylic acid ester group (—COOR²¹), a carboxylic acid anhydride group (—CO—O—CO—R²²), a hydroxamic acid group (—CO—NH(OH)), a N-substituted hydroxamic acid group (—CO—NR²³(OH)), a O-substituted hydroxamic acid group (—CO—NH(OR²⁴)), a carboxamide group (—CO—NH₂), a N-substituted or N,N-disubstituted carboxylic acid amide group, (—CO—NHR²⁵; —CO—NR²⁶R²⁷), an amido group (—HN—CO—R²⁸), a sulfonic acid group (—SO₃H), a sulfonamide group (—SO₂—NH₂), a N-substituted or N,N-disubstituted sulfonamide group (—SO₂—NHR²⁹; —SO₂—NR³⁰R³¹), an amidosulfone group (—NH—SO₂—R³²), a sulfone group (—SO₂—R³³), a phosphoric acid group (—OP(═O)(OH)₂), a phosphoric acid ester group (—OP(═O)(OR³⁴)(OR³⁵)), a phosphonic acid group (—P(═O)(OH)₂), an phosphonic acid ester group (—P(═O)(OR³)(OR³)), a halogen atom, a trifluormethyl group (—CF₃), a thiol group (—SH); a thioether group (—S—R³⁸), a hydroxy group (—OH); an alkoxy group (—O—R³⁹), a tetrazole group, an amino group (—NH₂), or a N-substituted or N,N-disubstituted amino group (—NHR⁴⁰; —NR⁴¹R⁴²);
which each independently can be substituted with one or more substituents, which can be the same or different; and,
wherein optionally, any two of the groups R¹, R², R³, R⁴, R⁵, R⁶, R⁷, R⁸, R⁹, R¹⁰, and R¹¹, as well the pairs R²⁶/R²⁷, R³⁰/R³¹, R³⁴/R³⁵, R³⁶/R³⁷ and R⁴¹/R⁴², independenly of each other, may form a part of a ring; and
wherein the substituents R²⁰, R²¹, R²², R²³, R²⁴, R²⁵, R²⁶, R²⁷, R²⁸, R²⁹, R³⁰, R³¹, R³², R³³, R³⁴R³⁵, R³⁶, R³⁷, R³⁸, R³⁹, R⁴⁰, R⁴¹, and R⁴² independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group.

4. The compound according to claim 1
wherein n is 0 or 1;
wherein R¹, R², R³, R⁴, R⁵, R⁶, R⁷, R⁷, R⁸, R⁹, R¹⁰, and R¹¹ independently of each other are
a hydrogen atom; or
a straight or branched chain, substituted or unsubstituted alkyl group comprising methyl (—CH₃) and ethyl (—C₂H₅); or
a halogen comprising a fluoro, chloro, bromo or iodo atom; or
a cyano group ; a thiol group; a hydroxy group; a carboxyl group, a tetrazole group, an amino group; an amido group;
and wherein EWG1 and EWG2 is a double bound oxygen (═O).

5. The compound according to claim 1,
wherein n is 0;
wherein R¹, R², R⁵, R⁶, R⁷, R⁸, R⁹, R¹⁰, and R¹¹, is each a hydrogen atom; and
wherein EWG1 and EWG2 is a double bound oxygen (═O).

6. The compound according to claim 1,
wherein n is 1;
wherein R¹, R², R³, R⁴, R⁵, R⁶, R⁷, R⁸, R⁹, R¹⁰, and R¹¹ is each a hydrogen atom; and
wherein EWG1 and EWG2 is a double bound oxygen (═O).

7. The compound according to claim 1,
wherein the group PM
has the formula (II) 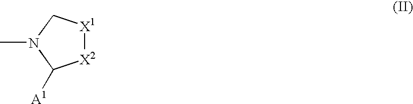
wherein X is CR⁵¹R⁵², O, S, or NR⁵; and
wherein X² is CR⁵⁴R⁵⁵, O, S, or NR⁵⁶; and
wherein R⁵¹, R⁵², R⁵³, R⁵⁴, R⁵⁵ and R⁵⁶, independently of each other, are
a hydrogen atom (—H); or an C₁, C₂, C₃, C₄, C₅, C₆, C₇, C₈ and Cg branched or straight chain alkyl, C₂, C₃, C₄, C₅, C₆, C₇, C₈ and Cg branched or straight chain alkenyl, C₂, C₃, C₄, C₅, C₆, C₇, C₈ and Cg branched or straight chain alkinyl, C₃, C₄, C₅, C₆, C₇, C₈ and Cg cycloalkyl, C₅, C₆, C₇, C₈ and Cg cycloalkenyl, aryl, heteroaryl or amino (—NH₂), or a N-substituted or N,N-disubstituted amino group (—NHR⁸⁰; —NR⁸R⁸²); and
which, independently of each other, can be substituted with one or more substituents, which can be the same or different; and,
wherein optionally, any two of the groups R⁵¹, R⁵², R⁵³, R⁵⁴, R⁵⁵, and R⁵⁶ if present, as well as the pairs R⁶⁶/R⁶⁷, R⁷⁰/R⁷¹, R⁷⁴/R⁷⁵, R⁷⁶/R⁷⁷ and R⁸¹/R⁸², independently of each other, may form a part of a ring; and
wherein the substituents R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, and R⁸², independently of each other, are a hydrogen atom (—H), or a C₁, C₂, C₃, C₄, C₅, C₆, C₇, C₈ and Cg branched or straight chain alkyl, aryl, heteroaryl, amino, halo, carbonyl, C₁, C₂, C₃, C₄, C₅, C₆, C₇, C₈ and Cg branched or straight chain alkoxy, C₂, C₃, C₄, C₅, C₆, C₇, C₈ and. C₉branched or straight chain alkenoxy, phenyloxy, benzyloxy, C₃, C₄, C₅, C₆, C₇, C₈ and C₉cycloalkyl, cyano, amido, thiol trifluoromethyl, or hydroxy group; and
wherein A¹ is
a hydrogen atom (—H) or a carbaldehyde (—CHO), a ketone group (—CO—R¹⁰⁰), a boronic acid group (—B(OH)₂), a cyano group (—C≡N), a carboxylic acid group (—COOH), a carboxylic acid ester group (—COOR¹⁰¹), a carboxylic acid anhydride group (—CO—O—CO—R¹⁰²), a hydroxamic acid group (—CO—NH(OH)), a N-substituted hydroxamic acid group (—CO—NR¹⁰³(OH)), a O-substituted hydroxamic acid group (—CO—NH(OR¹⁰⁴)), a carboxamide group (—CO—NH₂), a N-substituted or N,N-disubstituted carboxylic acid amide group, (—CO—NHR¹⁰⁵; —CO—NR¹⁰⁶R¹⁰⁷), an amido group (—HN—CO—R¹⁰⁸), a sulfonic acid group (—SO₃H), a sulfonamide group (—SO₂—NH₂), a N-substituted or N,N-disubstituted sulfonamide group (—SO₂—NHR¹⁰⁹; —SO₂—NR¹¹⁰OR¹¹¹), an amidosulfone group (—NH—SO₂—R¹¹²), a sulfone group (—SO₂—R¹¹³), a phosphoric acid group (—OP(═O)(OH)₂), a phosphoric acid ester group (—OP(═O)(OR¹¹⁴)(OR¹¹⁵)), a phosphonic acid group (—P(═O)(OH)₂), an phosphonic acid ester group (—P(═O)(OR¹¹⁶)(OR¹¹⁷)), a halogen atom, a trifluormethyl group (—CF₃), a thiol group (—SH); a thioether group (—S—R¹¹⁸), a hydroxy group (—OH); an alkoxy group (—O—R¹¹⁹), a tetrazole group, an amino group (—NH₂), or a N-substituted or N,N-disubstituted amino group (—NHR¹²⁰; —NR¹²¹, R¹²²); and
which, independently of each other, can be substituted with one or more substituents, which can be the same or different; and,
wherein optionally, the pairs R¹⁰⁶/R¹⁰⁷, R¹¹⁰/R¹¹¹, R¹¹⁴/R¹¹⁵, R¹¹⁶/R¹¹⁷ and R¹²¹/R¹²², independently of each other, may form a part of a ring; and
wherein the substituents R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R^{111 , R}¹¹², R¹¹³, R¹¹⁴, R¹¹⁵, R¹¹⁶, R¹¹⁷, R¹¹⁸, R¹¹⁹, R¹²⁰, R¹²¹, and R¹²², independently of each other, are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (III) 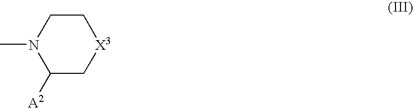
wherein X³ is CR¹³¹R¹³², O, S, or NR¹³³; and
wherein R¹³¹, R¹³², and R¹³³, independently of each other, are
a hydrogen atom (—H); or an C₁, C₂, C₃, C₄, C₅, C₆, C₇, C₈ and C₉branched or straight chain alkyl, C₂, C₃, C₄, C₅, C₆, C₇, C₈ and C₉branched or straight chain alkenyl, C₂, C₃, C₄, C₅, C₆, C₇, C₈ and Cg branched or straight chain alkinyl, C₃, C₄, C₅, C₆, C₇, C₈ and Cg cycloalkyl, C₅, C₆, C₇, C₈ and C₉ cycloalkenyl, aryl, heteroaryl or an amino group (—NH₂), or a N-substituted or N,N-disubstituted amino group (—N 160; —NR¹⁶¹R¹⁶²); and
which, independently of each other, can be substituted with one or more substituents, which can be the same or different; and,
wherein optionally, the the pair R¹³¹/R¹³², if present, as well the pairs R¹⁴⁶/R¹⁴⁷, R¹⁵⁰/R¹⁵¹, R¹⁵⁴/R¹⁵⁵, R¹⁵⁶/R¹ and R¹⁶¹/R¹⁶², independenly of each other, may form a part of a ring; and
wherein the substituents R¹⁴⁰, R¹⁴¹, R^{142 , R}¹⁴³, R¹⁴⁴, R¹⁴⁵, R¹⁴⁶, R¹⁴⁷, R¹⁴⁸, R¹⁴⁹, R¹⁵⁰, R¹⁵¹, R¹⁵², R¹⁵³, R¹⁵⁴, R¹⁵⁵, R¹⁵⁶, R¹⁵⁷, R¹⁵⁸, R¹⁵⁹, R¹⁶⁰, R¹⁶¹, and R¹⁶², independently of each other are a hydrogen atom (—H), or a C₁, C₂, C₃, C₄, Cs, C₆, C₇, C₈ and Cg branched or straight chain alkyl, aryl, heteroaryl, amino, halo, carbonyl, C₁, C₂, C₃, C₄, C₅, C₆, C₇, C₈ and Cg branched or straight chain alkoxy, C₂, C₃, C₄, Cs, C₆, C₇, C₈ and Cg branched or straight chain alkenoxy, phenyloxy, benzyloxy, C₃, C₄, C₅, C₆, C₇, C₈ and C₉ cycloalkyl, cyano, amido, thiol, trifluoromethyl, or hydroxy group; and
wherein A² is
a hydrogen atom (—H); or a carbaldehyde (—CHO), a ketone group (—CO—R¹⁸⁰), a boronic acid group (—B(OH)₂), a cyano group (—C≡N), a carboxylic acid group (—COOH), a carboxylic acid ester group (—COOR¹⁸¹), a carboxylic acid anhydride group (—CO—O—CO—R¹⁸²), a hydroxamic acid group (—CO—NH(OH)), a N-substituted hydroxamic acid group (—CO—NR¹⁸³ (OH)), a O-substituted hydroxainic acid group (—CO—NH(OR¹⁸⁴)), a carboxamide group (—CO—NH₂), a N-substituted or N,N-disubstituted carboxylic acid amide group, (—CO-NHR¹⁸⁵; —CO—NR¹⁸⁶R¹⁸⁷), an amido group (—HN—CO—R¹⁸⁸), a sulfonic acid group (—SO₃H), a sulfonamide group (—SO₂—NH₂), a N-substituted or N,N-disubstituted sulfonamide group (—SO₂—NHR⁸⁹; —SO₂—NR¹⁹⁰R¹⁹¹), an amidosulfone group (—NH—SO₂—R⁹²), a sulfone group (—SO₂—R¹⁹³), a phosphoric acid group (—OP(═O)(OH)₂), a phosphoric acid ester group (—OP(═O)(OR¹⁹⁴)(OR¹⁹⁵)), a phosphonic acid group (—P(═O)(OH)₂), an phosphonic acid ester group (—P(═O)(OR¹⁹⁶)(OR¹⁹⁷)), a halogen atom, a trifluormethyl group (—CF₃), a thiol group (—SH); a thioether group (—S—R¹⁹⁸), a hydroxy group (—OH); an alkoxy group (—O—R¹⁹⁹), a tetrazole group, an amino group (—NH₂), or a N-substituted or N,N-disubstituted amino group (—NHR²⁰⁰; —NR²⁰¹R²⁰²); and
which, independently of each other, can be substituted with one or more substituents, which can be the same or different; and,
wherein optionally, the pairs R¹⁸⁶/R¹⁸⁷, R¹⁹⁰/R¹⁹¹, R¹⁹⁴/R¹⁹⁵, R¹⁹⁶/R¹⁹⁷ and R²⁰¹/R²⁰² independenly of each other, may form a part of a ring; and
wherein the substituents R¹⁸⁰, R¹⁸¹, R¹⁸², R¹⁸³, R¹⁸⁴, R¹⁸⁵, R¹⁸⁶, R¹⁸⁷, R¹⁸⁸, R¹⁸⁹, R¹⁹⁰, R¹⁹¹, R¹⁹², R¹⁹³, R¹⁹⁴, R¹⁹⁵, R¹⁹⁶, R¹⁹⁹⁷, R¹⁹⁸, R¹⁹⁹, R²⁰⁰, R²⁰¹, and R²⁰², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (IV) 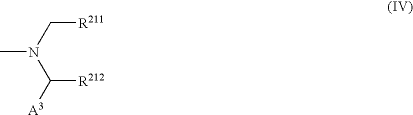
wherein R²¹¹ and R²¹², independently of each other, are
a hydrogen atom (—H); or an C₁, C₂, C₃, C₄, C₅, C₆, C₇, C₈ and C₉branched or straight chain alkyl, C₂, C₃, C₄, C₅, C₆, C₇, C₈ and C₉branched or straight chain alkenyl, C₂, C₃, C₄, C₅, C₆, C₇, C₈ and C₉branched or straight chain alkinyl, C₃, C₄, C₅, C₆, C₇, C₈ and Cg cycloalkyl, C₅, C₆, C₇, C₈ and Cs cycloalkenyl, aryl, heteroaryl or an amino group (—NH₂), or a N-substituted or N,N-disubstituted amino group (—NHR²⁴⁰; —NR²⁴¹R²⁴²); and
which, independently of each other, can be substituted with one or more substituents, which can be the same or different; and,
wherein optionally, the pairs R²²⁶/R²²⁷, R²³⁰/R²³¹, R²³⁴/R²³⁵, R²³⁶/R²³⁷ and R²⁴/R²⁴² independenly of each other, may form a part of a ring; and
wherein the substituents R²²⁰, R²²¹, R²²², R²²³, R²²⁴, R²²⁵, R²²⁶, R²²⁷, R²²⁸, R²²⁹, R²³⁰, R²³¹, R²³², R²³³, R²³⁴, R²³⁵, R²³⁶, R²³⁷, R²³⁸, R²³⁹, R²⁴⁰, R²⁴¹, and R²⁴², independently of each other, are a hydrogen atom (—H), or a C₁, C₂, C₃, C₄, C₅, C₆, C₇, C₈ and C₉branched or straight chain alkyl, aryl, heteroaryl, amino, halo, carbonyl, C₁, C₂, C₃, C₄, C₅, C₆, C₇, C₈ and C₉branched or straight chain alkoxy, C₂, C₃, C₄, C₅, C₆, C₇, C₈ and Cg branched or straight chain alkenoxy, phenyloxy, benzyloxy, C₃, C₄, C₅, C₆, C₇, C₈ and Cs cycloalkyl, cyano, amido, thiol, trifluoromethyl, or hydroxy group; and
wherein A³ is
a hydrogen atom (—H); or a carbaldehyde (—CHO), a ketone group (—CO—R²⁶⁰), a boronic acid group (—B(OH)₂), a cyano group (—C═N), a carboxylic acid group (—COOH), a carboxylic acid ester group (—COOR²⁶¹), a carboxylic acid anhydride group (—CO—O—CO—R²⁶²), a hydroxamic acid group (—CO—NH(OH)), a N-substituted hydroxamic acid group (—CO—NR⁶³ (OH)), a O-substituted hydroxamic acid group (—CO—NH(OR²⁶⁴)), a carboxamide group (—CO—NH₂), a N-substituted or N,N-disubstituted carboxylic acid amide group, (—CO—NHR²⁶⁵; —CO—NR²⁶⁶, R²⁶⁷), an amido group (—HN—CO—R²⁶⁸), a sulfonic acid group (—SO₃H), a sulfonamide group (—SO₂—NH₂), a N-substituted or N,N-disubstituted sulfonamide group (—SO₂—NHR²⁶¹; —SO₂—NR²⁷⁰R²⁷¹), an amidosulfone group (—NH—SO₂—R²⁷²), a sulfone group (—SO₂—R²⁷³), a phosphoric acid group (—OP(═O)(OH)₂), a phosphoric acid ester group (—OP(═O)(OR²⁷⁴)(OR²⁷⁵)), a phosphonic acid group (—P(═O)(OH)₂), an phosphonic acid ester group (—P(═O)(OR²⁷⁶)(OR²⁷⁷)), a halogen atom, a trifluormethyl group (—CF₃), a thiol group (—SH); a thioether group (—S—R²⁷⁸), a hydroxy group (—OH); an alkoxy group (—O—R²⁷⁹), a tetrazole group, an amino group (—NH₂), or a N-substituted or N,N-disubstituted amino group (—NHR²⁸⁰; —NR²⁸R²⁸²); and
which, independently of each other, can be substituted with one or more substituents, which can be the same or different; and,
wherein optionally, the pairs R²⁶⁶/R²⁶⁷, R²⁷⁰/R²⁷¹, R²⁷⁴/R²⁷⁵, R²⁷⁶/R²⁷⁷ and R²⁸/R²⁸² independenly of each other, may form a part of a ring; and
wherein the substituents R²⁶⁰, R²⁶¹, R²⁶², R²⁶³, R²⁶⁴, R²⁶⁵, R²⁶⁶, R²⁶⁷, R²⁶⁸, R²⁶⁹, R²⁷⁰, R²⁷¹, R²⁷², R²⁷³, R²⁷⁴, R²⁷⁵, R²⁷⁶, R²⁷⁷, R²⁷⁸, R²⁷⁹, R²⁸⁰, R²⁸¹, and R²⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (V) 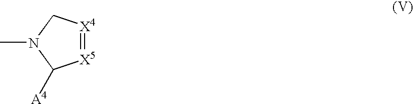
wherein X⁴ is CR²⁹¹ or N; and
wherein X⁵ is CR²⁹² or N; and
wherein R²⁹¹ and R²⁹², independently of each other, are
a hydrogen atom (—H); or an C₁, C₂, C₃, C₄, C₅, C₆, C₇, C₈ and Cg branched or straight chain alkyl, C₂, C₃, C₄, C₅, C₆, C₇, C₈ and Cg branched or straight chain alkenyl, C₂, C₃, C₄, C₅, C₆, C₇, C₈ and Cg branched or straight chain alkinyl, C₃, C₄, Cs, C₆, C₇, C₈ and Cg cycloalkyl, Cs, C₆, C₇, C₈ and Cs cycloalkenyl, aryl, heteroaryl group, or an amino group (—NH₂), or a N-substituted or N,N-disubstituted amino group (—NHR³²⁰; —NR³²¹R³²²); and
which, independently of each other, can be substituted with one or more substituents, which can be the same or different; and,
wherein optionally, the the pair R²⁹¹/R²⁹², if present, as well the pairs R³⁰⁶/R³⁰⁷, R³¹⁰/R³¹¹, R³¹⁴/R³¹⁵, R³¹⁶/R³¹⁷ and R³²¹/R³²², independenly of each other, may form a part of a ring; and
wherein the substituents R³⁰⁰, R³⁰¹, R³⁰², R³⁰³, R³⁰⁴, R³⁰⁵, R³⁰⁶, R³⁰⁷, R³⁰⁸, R³⁰⁹, R³¹⁰, R³¹¹, R³¹², R³¹³, R³¹⁴, R³¹⁵, R³¹⁶, R³¹⁷, R³¹⁸, R³¹⁹, R³²⁰, R³²¹, and R³²², independently of each other are a hydrogen atom (—H), or a C₁, C₂, C₃, C₄, Cs, C₆, C₇, C₈ and Cg branched or straight chain alkyl, aryl, heteroaryl, amino, halo, carbonyl, C₁, C₂, C₃, C₄, C₅, C₆, C₇, C₈ and C₉branched or straight chain alkoxy, C₂, C₃, C₄, C₅, C₆, C₇, C₈ and Cg branched or straight chain alkenoxy, phenyloxy, benzyloxy, C₃, C₄, Cs, C₆, C₇, C₈ and Cg cycloalkyl, cyano, amido, thiol, trifluoromethyl, or hydroxy group; and
wherein A⁴ is
a hydrogen atom (—H); or a carbaldehyde (—CHO), a ketone group (—CO—R³⁴⁰), a boronic acid group (—B(OH)₂), a cyano group (—C≡N), a carboxylic acid group (—COOH), a carboxylic acid ester group (—COOR³⁴¹), a carboxylic acid anhydride group (—CO—O—CO—R³⁴²), a hydroxamic acid group (—CO—NH(OH)), a N-substituted hydroxamic acid group (—CO—NR³⁴³(OH)), a O-substituted hydroxamic acid group (—CO—NH(OR³⁴⁴)), a carboxamide group (—CO—NH₂), a N-substituted or N,N-disubstituted carboxylic acid amide group, (—CO—NHR³⁴⁵; —CO—NR³⁴⁶R³⁴⁷), an amido group (—HN—CO—R³⁴⁸), a sulfonic acid group (—SO₃H), a sulfonamide group (—SO₂—NH₂), a N-substituted or N,N-disubstituted sulfonamide group (—SO₂—NHR³⁴⁹; —SO₂—NR³⁵° R³⁵¹), an amidosulfone group (—NH—SO₂—R⁵¹), a sulfone group (—SO₂—R⁵³), a phosphoric acid group (—OP(═O)(OH)₂), a phosphoric acid ester group (—OP(═O)(OR³⁵⁴)(OR³⁵⁵)), a phosphonic acid group (—P(═O)(OH)₂), an phosphonic acid ester group (—P(═O)(OR³⁵⁶)(OR³⁵⁷)), a halogen atom, a trifluormethyl group (—CF₃), a thiol group (—SH); a thioether group (—S—R³⁵⁸), a hydroxy group (—OH); an alkoxy group (—O—R³⁵⁹), a tetrazole group, an amino group (—NH₂), or a N-substituted or N,N-disubstituted amino group (—NHR³⁶⁰; —NR³⁶¹R³⁶²); and
which, independently of each other, can be substituted with one or more substituents, which can be the same or different; and,
wherein optionally, the pairs R³⁴⁶/R³⁴⁷, R³⁵⁰/R³⁵¹, R³⁵⁴/R³⁵⁵, R³⁵⁶/R³⁵⁷ and R³⁶/R³⁶² independenly of each other, may form a part of a ring; and
wherein the substituents R³⁴⁰, R³⁴¹, R³⁴², R³⁴³, R³⁴⁴, R³⁴⁵, R³⁴⁶, R³⁴⁷, R³⁴⁸, R³⁴⁹, R³⁵⁰, R³⁵¹, R³⁵², R³⁵³, R³⁵⁴, R³⁵⁵, R³⁵⁶, R³⁵⁷, R³⁵⁸, R³⁵⁹, R³⁶⁰, R³⁶¹, and R³⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (VI) 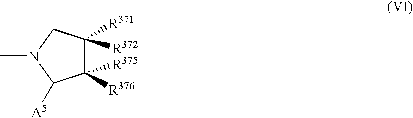
wherein R³⁷¹, R³⁷², R³⁷⁵ and R³⁷⁶, independently of each other, a hydrogen atom (—H); or a C₁, C₂, C₃, C₄, C₅, C₆, C₇, C₈ and C₉branched or straight chain alkyl, C₂, C₃, C₄, C₅, C₆, C₇, C₈ and Cg branched or straight chain alkenyl, C₂, C₃, C₄, C₅, C₆, C₇, C₈ and Cg branched or straight chain alkinyl, C₃, C₄, C₅, C₆, C₇, C₈ and Cg cycloalkyl, C₅, C₆, C₇, C₈ and Cg cycloalkenyl, aryl, heteroaryl, aryl-alkyl, heteroaryl-alkyl, aryl-heteroalkyl, heteroaryl-heteroalkyl group or, a carbaldehyde (—CHO), a ketone group (—CO—R³⁸⁰), a boronic acid group (—B(OH)₂), a cyano group (—C≡N), a carboxylic acid group (—COOH), a carboxylic acid ester group (—COOR³⁸¹), a halogen atom, a trifluormethyl group (—CF₃), a thiol group (—SH); a thioether group (—S—R³⁹⁸), a hydroxy group (—OH); an alkoxy group (—O—R³⁹⁹), a tetrazole group, an amino group (—NH₂), or a N-substituted or N,N-disubstituted amino group (—NR⁴⁰⁰; —NR⁴⁰R⁴⁰²); and
which, independently of each other, can be substituted with one or more substituents, which can be the same or different; and,
wherein optionally, any two of the groups R³⁷¹, R³⁷², R³⁷⁵, and R³⁷⁶, as well as the pairs R³⁸⁶/R³⁸⁷, R³⁹⁰/R³⁹¹, R³⁹⁴/R³⁹⁵, R³⁹⁶/R³⁹⁷ and R⁴⁰¹/R⁴⁰², independenly of each other, may form a part of a ring; and
wherein the substituents R³⁸⁰, R³⁸¹, R³⁸², R³⁸³, R³⁸⁴, R³⁸⁵, R³⁸⁶, R³⁸⁷, R³⁸⁸, R³⁸⁹, R³⁹⁰, R³⁹¹, R³⁹², R³⁹³, R³⁹⁴, R³⁹⁵, R³⁹⁶, R³⁹⁷, R³⁹⁸, R³⁹⁹, R⁴⁰⁰, R⁴⁰¹, and R⁴⁰², independently of each other are a hydrogen atom (—H), or a C₁, C₂, C₃, C₄, C₅, C₆, C₇, C₈ and Cg branched or straight chain alkyl, aryl, heteroaryl, amino, halo, carbonyl, C₁, C₂, C₃, C₄, C₅, C₆, C₇, C₈ and C₉ branched or straight chain alkoxy, C₂, C₃, C₄, C₅, C₆, C₇, C₈ and Cg branched or straight chain alkenoxy, phenyloxy, benzyloxy, C₃, C₄, C₅, C₆, C₇, C₈ and Cg cycloalkyl, cyano, amido, thiol, trifluoromethyl, or hydroxy group; and
alternatively; the two groups R³⁷¹ and R³⁷² can be together an oxo (═O) or hydroxyimino (═N—OH) group; and
alternatively; the two groups R³⁷⁵ and R³⁷⁶ can be together an oxo (═O) or hydroxyimino (═N—OH) group; and
wherein A⁵ is
a hydrogen atom (—H); or a carbaldehyde (—CHO), a ketone group (—CO—R⁴²⁰), a boronic acid group (—B(OH)₂), a cyano group (—C≡N), a carboxylic acid group (—COOH), a carboxylic acid ester group (—COOR⁴²¹), a carboxylic acid anhydride group (—CO—O—CO—R⁴²²), a hydroxamic acid group (—CO—NH(OH)), a N-substituted hydroxamic acid group (—CO—NR⁴²³(OH)), a O-substituted hydroxamic acid group (—CO—NH(OR⁴²⁴)), a carboxamide group (—CO—NH₂), a N-substituted or N,N-disubstituted carboxylic acid amide group, (—CO—NHR⁴²⁵; —CO—NR⁴²⁶R⁴²⁷), an amido group (—HN—CO—R⁴²⁸), a sulfonic acid group (—SO₃H), a sulfonamide group (—SO₂—NH₂), a N-substituted or N,N-disubstituted sulfonamide group (—SO₂—NHR⁴²⁹;
—SO₂—NR⁴³⁰R⁴³¹), an amidosulfone group (—NH—SO₂—R⁴³²), a sulfone group (—SO₂—R⁴³³), a phosphoric acid group (—OP(═O)(OH)₂), a phosphoric acid ester group (—OP(═O)(OR⁴³⁴)(OR⁴³⁵)), a phosphonic acid group (—P(═O)(OH)₂), an phosphonic acid ester group (—P(═O)(OR⁴³⁶)(OR⁴³⁷)), a halogen atom, a trifluormethyl group (—CF₃), a thiol group (—SH); a thioether group (—S—R⁴³⁸), a hydroxy group (—OH); an alkoxy group (—O—R⁴³⁹), a tetrazole group, an amino group (—NH₂), or a N-substituted or N,N-disubstituted amino group (—NHR⁴⁴⁰; —NR⁴⁴R⁴⁴²); and
which, independently of each other, can be substituted with one or more substituents, which can be the same or different; and,
wherein optionally, the pairs R⁴²⁶/R⁴²⁷, R⁴³⁰/R⁴³¹, R⁴³⁴/R⁴³⁵, R⁴³⁶/R⁴³⁷ and R⁴⁴¹/R⁴⁴² independenly of each other, may form a part of a ring; and
wherein the substituents R⁴²⁰, R⁴²¹, R⁴²², R⁴²³, R⁴²⁴, R⁴²⁵, R⁴²⁶, R⁴²⁷, R⁴²⁸, R⁴²⁹, R⁴³⁰R⁴³¹, R⁴³², R⁴³³, R⁴³⁴, R⁴³⁵, R⁴³⁶, R⁴³⁷, R⁴³⁸, R⁴³⁹, R⁴⁴⁰, R⁴⁴¹, and R⁴⁴², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (VII) 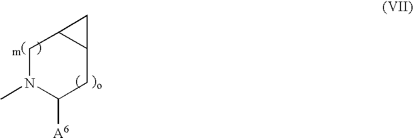
wherein m is equal to 1 or 2, and o is equal to 1 or 2, and m or o can be 0;
wherein A⁶ is a hydrogen atom (—H); or a carbaldehyde (—CHO), a ketone group (—CO—R⁴⁶⁰), a boronic acid group (—B(OH)₂), a cyano group (—C≡N), a carboxylic acid group (—COOH), a carboxylic acid ester group (—COOR⁴⁶¹), a carboxylic acid anhydride group (—CO—O—CO—R⁴⁶²), a hydroxamic acid group (—CO—NH(OH)), a N-substituted hydroxamic acid group (—CO—NR⁴⁶³(OH)), a O-substituted hydroxamic acid group (—CO—NH(OR⁴⁶⁴)), a carboxamide group (—CO—NH₂), a N-substituted or N,N-disubstituted carboxylic acid amide group, (—CO—NHR⁴⁶⁵; —CO—NR⁴⁶⁶R⁴⁶⁷), an amido group (—HN—CO—R⁴⁶⁸), a sulfonic acid group (—SO₃H), a sulfonamide group (—SO₂—NH₂), a N-substituted or N,N-disubstituted sulfonamide group (—SO₂—NHR⁴⁶⁹; —SO₂—NR⁷⁰R⁴⁷), an amidosulfone group (—NH—SO₂—R⁴⁷²), a sulfone group (—SO₂—R⁴⁷³), a phosphoric acid group (—OP(═O)(OH)₂), a phosphoric acid ester group (—OP(═O)(OR⁴⁷⁴)(OR⁴⁷⁵)), a phosphonic acid group (—P(═O)(OH)₂), an phosphonic acid ester group (—P(═O)(OR⁴⁷⁶)(OR⁴⁷⁷)), a halogen atom, a trifluormethyl group (—CF₃), a thiol group (—SH); a thioether group (—S—R⁴⁷⁸), a hydroxy group (—OH); an alkoxy group (—O—R⁴⁷⁹), a tetrazole group, an amino group (—NH₂), or a N-substituted or N,N-disubstituted amino group (—NHR⁴⁸⁰; —NR⁸R⁴⁸²);
which, independently of each other, can be substituted with one or more substituents, which can be the same or different; and,
wherein optionally, the pairs R⁴⁶⁶/R⁴⁶⁷, R⁴⁷⁰/R⁴⁷, R⁴⁷⁴/R⁴⁷⁵, R⁴⁷⁶/R⁴⁷⁷ and R⁴⁸¹/R⁴⁸² independenly of each other, may form a part of a ring; and
wherein the substituents R⁴⁶⁰, R⁴⁶¹, R⁴⁶², R⁴⁶³, R⁴⁶⁴, R⁴⁶⁵, R⁴⁶⁶, R⁴⁶⁷, R⁴⁶⁸, R⁴⁶⁹, R⁴⁷⁰, R⁴⁷¹, R⁴⁷², R⁴⁷³, R⁴⁷⁴, R⁴⁷⁵, R⁴⁷⁶, R⁴⁷⁷, R⁴⁷⁸, R⁴⁷⁹, R⁴⁸⁰, R⁴⁸¹, and R⁴⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (VIII) 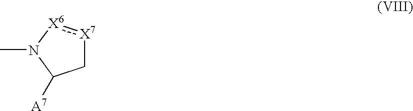
wherein X⁶ is selected from CR⁴⁹⁰R⁴⁹¹, O, S or NR⁴⁹², when the bond between X⁶ and X⁷ is a single bond; and
wherein X⁷ is selected from CR⁴⁹³R⁴⁹⁴, O, S, or NR⁴⁹⁵, when the bond between X⁶ and X⁷ is a single bond;
or alternatively,
wherein X⁶ is selected from CR⁴⁹⁶ or N, when the bond between X⁶ and X⁷ is a double bond; and
wherein X⁷ is selected from CR⁴⁹⁷ or N, when the bond between X⁶ and X⁷ is a double bond; and
wherein R⁴⁹⁰, R⁴⁹¹, R⁴⁹², R⁴⁹³, R⁴⁹⁴, R⁴⁹⁵, R⁴⁹⁶, and R⁴⁹⁷, independently of each other, are a hydrogen atom (—H); or a C₁, C₂, C₃, C₄, C₅, C₆, C₇, C₈ and C₉ branched or straight chain alkyl, C₂, C₃, C₄, C₅, C₆, C₇, C₈ and C₉ branched or straight chain alkenyl, C₂, C₃, C₄, C₅, C₆, C₇, C₈ and Cg branched or straight chain alkinyl, C₃, C₄, C₅, C₆, C₇, C₈ and C₉ cycloalkyl, C₅, C₆, C₇, C₈ and C₉ cycloalkenyl, heteroalkyl, aryl, heteroaryl, aryl-alkyl, heteroaryl-alkyl, aryl-heteroalkyl, heteroaryl-heteroalkyl group or, a tetrazole group, an amino group (—NH₂), or a N-substituted or N,N-disubstituted amino group (—NHR⁵²⁰; —NR⁵²¹R⁵²²); and
which, independently of each other, can be substituted with one or more substituents, which can be the same or different; and,
wherein optionally, any two the groups R⁴⁹⁰, R⁴⁹¹, R⁴⁹², R⁴⁹³, R⁴⁹⁴, R⁴⁹⁵, R⁴⁹⁶, and R⁴⁹⁷, if present, as well as the pairs R⁵⁰⁶/R⁵⁰⁷, R⁵¹⁰/R⁵¹¹, R⁵¹⁴/R⁵¹⁵, R⁵¹⁶/R⁵¹⁷ and R⁵²/R⁵²² independenly of each other, may form a part of a ring; and
wherein the substituents R⁵⁰⁰, R⁵⁰¹, R⁵⁰², R⁵⁰³, R⁵⁰⁴, R⁵⁰⁵, R¹⁶, R⁵⁰⁷, R⁵⁰⁸, R⁵⁰⁹, R⁵¹⁰R⁵¹¹, R⁵¹², R⁵¹³, R⁵¹⁴, R⁵¹⁵, R⁵¹⁶, R⁵¹⁷, R⁵¹⁸, R⁵¹⁹, R⁵²⁰, R⁵²¹, and R⁵²², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group; and
wherein A⁷ is
a hydrogen atom (—H); or a carbaldehyde (—CHO), a ketone group (—CO—R⁵⁴⁰), a boronic acid group (—B(OH)₂), a cyano group (—C≡N), a carboxylic acid group (—COOH), a carboxylic acid ester group (—COOR⁵⁴¹), a carboxylic acid anhydride group (—CO—O—CO—R⁵⁴²), a hydroxamic acid group (—CO—NH(OH)), a N-substituted hydroxamic acid group (—CO—NR⁵⁴³(OH)), a O-substituted hydroxamic acid group (—CO—NH(OR⁵⁴⁴)), a carboxamide group (—CO—NH₂), a N-substituted or N,N-disubstituted carboxylic acid amide group, (—CO—NHR⁵⁴⁵; —CO—NR⁵⁴⁶R⁵⁴⁷), an amido group (—HN—CO—R⁵⁴⁸), a sulfonic acid group (—SO₃H), a sulfonamide group (—SO₂—NH₂), a N-substituted or N,N-disubstituted sulfonamide group (—SO₂—NHR⁵⁴⁹;
—SO₂—NR⁵⁵, R⁵⁵¹), an amidosulfone group (—NH—SO₂—R⁵⁵²), a sulfone group (—SO₂—R⁵⁵³), a phosphoric acid group (—OP(═O)(OH)₂), a phosphoric acid ester group (—OP(═O)(OR⁵⁵⁴)(OR⁵⁵⁵)), a phosphonic acid group (—P(═O)(OH)₂), an phosphonic acid ester group (—P(═O)(OR⁵⁵⁶)(OR⁵⁵⁷)), a halogen atom, a trifluormethyl group (—CF₃), a thiol group (—SH); a thioether group (—S—R⁵⁵⁸), a hydroxy group (—OH); an alkoxy group (—O—R⁵⁵⁹), a tetrazole group, an amino group (—NH₂), or a N-substituted or N,N-disubstituted amino group (—NHR⁵⁶⁰; —NR⁵⁶¹R⁵⁶²); and
which, independently of each other, can be substituted with one or more substituents, which can be the same or different; and,
wherein optionally, the pairs R⁵⁴⁶/R⁵⁴⁷, R⁵⁵⁰/R⁵⁵¹, R⁵⁵⁴/R⁵⁵⁵, R⁵⁵⁶/R⁵⁵⁷ and R⁵⁶¹¹R⁵⁶², independenly of each other, may form a part of a ring; and
wherein the substituents R⁵⁴⁰, R⁵⁴¹, R⁵⁴², R⁵⁴³, R⁵⁴⁴, R⁵⁴⁵, R⁵⁴⁶, R⁵⁴⁷, R⁵⁴⁸, R⁵⁴⁹, R^{550 R}⁵⁵¹, R⁵⁵², R⁵⁵³, R⁵⁵⁴, R⁵⁵⁵, R⁵⁵⁶, R⁵⁵⁷, R⁵⁵⁸, R⁵⁵⁹, R⁵⁶⁰, R⁵⁶¹, and R⁵⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (IX) or (IXa) 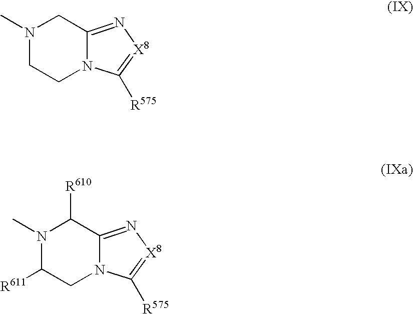
wherein X is N or CR⁵⁷⁰; and
wherein R⁵⁷⁰, R⁵⁷⁵, R⁶¹⁰ and R⁶¹¹ independently of each other, are a hydrogen atom (—H); or an C₁, C₂, C₃, C₄, C₅, C₆, C₇, C₈ and Cg branched or straight chain alkyl, C₂, C₃, C₄, C₅, C₆, C₇, C₈ and Cg branched or straight chain alkenyl, C₂, C₃, C₄, C₅, C₆, C₇, C₈ and C₉ branched or straight chain alkinyl, C₃, C₄, C₅, C₆, C₇, C₈ and C₉cycloalkyl, Cs, C₆, C₇, C₈ and C₉cycloalkenyl, aryl, heteroaryl, aryl-alkyl, aryl-heteroalkyl group or, a carbaldehyde (—CHO), a ketone group (—CO—R⁵⁸⁰), a boronic acid group (—B(OH)₂), a cyano group (—C≡N), a carboxylic acid group (—COOH), a carboxylic acid ester group (—COOR⁵⁸¹), a carboxylic acid anhydride group (—CO—O—CO—R⁵⁸²), a hydroxamic acid group (—CO—NH(OH)), a N-substituted hydroxamic acid group (—CO—NR⁵⁸³(OH)), a O-substituted hydroxamic acid group (—CO—NH(OR⁵¹¹)), a carboxamide group (—CO—NH₂), a N-substituted or N,N-disubstituted carboxylic acid amide group, (—CO—NHR⁵⁸⁵; —CO—NR⁵⁸⁶R⁵⁸⁷), an amido group (—HN—CO—R⁵⁸⁸), a sulfonic acid group (—SO₃H), a sulfonamide group (—SO₂—NH₂), a N-substituted or N,N-disubstituted sulfonamide group (—SO₂—NHR⁵⁸⁹; —SO₂—NR⁵⁹¹R⁵⁹¹), an amidosulfone group (—NH—SO₂—R⁵⁹²), a sulfone group (—SO₂—R⁵⁹³), a phosphoric acid group (—OP(═O)(OH)₂), a phosphoric acid ester group (—OP(═O)(OR⁵⁹⁴)(OR⁵⁹⁵)), a phosphonic acid group (—P(═O)(OH)₂), an phosphonic acid ester group (—P(═O)(OR⁵⁹⁶)(OR⁵⁹⁷)), a halogen atom, a trifluormethyl group (—CF₃), a thiol group (—SH); a thioether group (—S—R⁵⁹⁸), a hydroxy group (—OH); an alkoxy group (—O—R⁵⁹⁹), a tetrazole group, an amino group (—NH₂), or a N-substituted or N,N-disubstituted amino group (—NHR⁶⁰⁰; —NR⁶⁰¹R⁶⁰²);
which, independently of each other, can be substituted with one or more substituents, which can be the same or different; and,
wherein optionally, the pairs R⁵⁷⁰/R⁵⁷⁵, if present, as well as the pairs R⁵⁸⁶/R⁵⁸⁷, R⁵⁹⁰/R⁵⁹¹, R⁵⁹⁴/R⁵⁹⁵, R⁵⁹⁶/R⁵⁹⁷ and R⁶⁰/R⁶⁰², independenly of each other, may form a part of a ring; and
wherein the substituents R⁵⁸⁰, R⁵⁸¹, R⁵⁸², R⁵⁸³, R⁵⁸⁴, R⁵⁸⁵, R⁵⁸⁶, R⁵⁸⁷, R⁵⁸⁸, R⁵⁸⁹, R⁵⁹⁰R⁵⁹¹, R⁵⁹², R⁵⁹³, R⁵⁹⁴, R⁵⁹⁵, R⁵⁹⁶, R⁵⁹⁷, R⁵⁹⁸, R⁵⁹⁹, R⁶⁰⁰, R⁶⁰¹, and R⁶⁰², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (X) 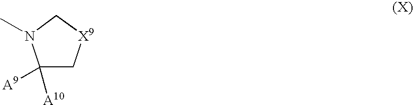
wherein the groups X⁹ is CR⁹⁰R⁹⁰¹, S, SO, SO_{2 or NR}⁹⁰²
wherein R⁹⁰⁰, R⁹⁰¹ and R⁹⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR⁹¹⁰R⁹¹¹.
wherein A⁹ and A¹⁰ are, independently of each other, selected from hydrogen, cyano, —C(═O)NR⁹¹², R⁹¹³, or C₁, C₂, C₃, C₄, C₅or C₆alkyl, which is linear or branched and is optionally substituted with 1, 2, 3, 4, or 5 halogens;
wherein
R⁹¹⁰ and R⁹¹², are, independently of each other, selected from hydrogen, or C₁, C₂, C₃, C₄, C₅or C₆alkyl, which is linear or branched and is optionally substituted with 1, 2, 3, 4, or 5 halogens; and
R⁹¹¹ and R⁹¹³, are, independently of each other, selected from the group consisting of
(1) phenyl, which is optionally substituted with 1, 2, 3, 4, or 5, substituents independently selected from halogen and R⁹²⁰;
(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of
(a) hydroxy,
(b) —COOH,
(c) —COO(C₁, C₂, C₃, C₄, C₅or C₆alkyl), i.e. ester,
(d) phenyl,
(e) naphthyl,
(f) C₃, C₄, C₅or C₆cycloalkyl,
(g) a 5- or 6 membered heterocycle which may be saturated or unsaturated comprising 1, 2, 3, or 4 heteroatoms independently selected from nitrogen , oxygen or sulfur;
(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;
wherein said C₃, C₄, C₅or C₆cycloalkyl, phenyl, naphthyl, are optionally substituted with 1, 2, 3, 4, or 5 substituents independently selected from halogen and R⁹²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R⁹²⁰; and
(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;
wherein R⁹²⁰ is selected from the group consisting of:
(1) hydroxy;
(2) cyano;
(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;
(4) C₁, C₂, C₃, C₄, C₅, C₆, C₇, C₈, C₉ or C₁₀ alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from
(a) hydroxy;
(b) —COOH;
(c) —COO(C₁, C₂, C₃, C₄, C₅or C₆alkyl) i.e. ester, which may linear or branched and is optionally substituted with 1, 2, 3, 4, or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅, or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(f) —CONR⁹²⁵, R⁹²⁵;
(g) —SO₂NR⁹²⁵, R⁹²⁵;
(h) —NR²⁵—C(═O)R⁹²⁵
(i) —NR⁹²⁵—C(═O)NR⁹²⁵R⁹²⁵;
(j) —NR⁹²⁵ COOR⁹³⁰
(k) —O—CO—R⁹³⁰
(l) —O—CO—NR⁹²⁵R⁹²⁵;
(m) —NR⁹²⁵SO₂R⁹³⁰;
(n) —NR⁹²⁵R⁹²⁵;
(o) phenyl which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and
(p) C₃, C₄C₅or C₆cycloalkyl, which is optionally substituted with 1, 2, 3, 4, 5, or 6 halogens;
(5) OC₁, OC₂, OC₃, OC₄, OC₅, OC₆, OC₇, OC₈, OC₉or OC₁₀alkyl, which is linear or branched and is optionally substituted with 0, 1, 2, 3, 4, or 5 halogen atoms and 0, 1, or 2 substitutents selected from
(a) hydroxy;
(b) —COOH;
(c) —COO(C₁, C₂, C₃, C₄, C₅or C₆alkyl) i.e. ester, which may be linear or branched and is optionally substituted with 1, 2, 3, 4 or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, Cs or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(f) —CONR⁹²⁵, R⁹²⁵;
(g) —SO₂NR⁹²⁵R⁹²;
(h) —NR⁹²⁵—C(═O)R⁹²⁵
(i) —NR⁹²⁵—C(═O)NR⁹²⁵R⁹²⁵
(j)—NR⁹²⁵COOR⁹³⁰
(k) —O—CO—R⁹³⁰
(l) —O—CO—NR⁹²⁵, R⁹²⁵;
(m) —NR⁹²⁵SO₂R⁹³⁰;
(n) NR⁹²⁵R⁹²⁵;
(o) phenyl, which is optionally substituted with 1, 2, 3, 4, or 5 groups independently selected from halogen, hydroxy, C₁, C₂, C₃, C₄, C₅ or C₆alkyl, —OC₁, —OC₂, —OC₃, -OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and
(p) C₃, C₄C₅or C₆cycloalkyl, which is optionally substituted with 1, 2, 3, 4, 5, or 6 halogens;
(6) —COOH;
(7) —COO(C₁, C₂, C₃, C₄, C₅or C₆alkyl) i.e. ester, which may be linear or branched and is optionally substituted with 1, 2, 3, 4, 5 halogens;
(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.
(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(10) —CONR⁹²⁵R⁹²⁵ (11)—SO₂NR⁹²⁵R⁹²⁵;
(12) —NR⁹²⁵—C(═O)R⁹²⁵
(13)—NR⁹²⁵—C(═O)NR⁹²⁵R⁹²⁵;
(14) —NR⁹²⁵COOR⁹³⁰
(15)—O—CO—R⁹³⁰
(16) —O—CO—NR⁹²⁵, R⁹²⁵;
(17) —NR⁹²⁵SO₂R⁹³⁰;
(18)—NR⁹²⁵R⁹²⁵;
(19) phenyl , which is optionally substituted with 1, 2, 3, 4, or 5 group independently selected from halogen, hydroxy, C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;
wherein R⁹³⁰ is selected from the group consisting of phenyl, C₃, C₄C₅or C₆ cycloalkyl, and C₃, C₄Cs or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.
wherein R⁹²⁵ is selected from R⁹³⁰ and hydrogen.
wherein the group PM
has the formula (XI) 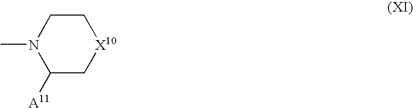
wherein the groups X¹⁰ is CR¹⁰⁰⁰R¹⁰⁰¹, S, SO, SO₂or NR¹⁰⁰²
wherein R¹⁰⁰⁰, R¹⁰⁰¹, and R¹⁰⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR¹⁰¹⁰R¹⁰¹¹.
and A¹¹ is selected from hydrogen, cyano, —C(═O)NR¹⁰¹²R¹⁰¹³, or C₁, C₂, C₃, C₄, C₅or C₆alkyl, which is linear or branched and is optionally substituted with 1, 2, 3, 4, or 5 halogens;
wherein
R¹⁰¹⁰ and R¹⁰¹², are, independently of each other, selected from hydrogen, or C₁, C₂, C₃, C₄, C₅or C₆alkyl, which is linear or branched and is optionally substituted with 1, 2, 3, 4, or 5 halogens; and
R¹⁰¹¹ and R¹⁰¹³, are, independently of each other, selected from the group consisting of
(1) phenyl, which is optionally substituted with 1, 2, 3, 4, or 5, substituents independently selected from halogen and R¹⁰²⁰;
(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of
(a) hydroxy,
(b) —COOH,
(c) —COO(C₁, C₂, C₃, C₄, C₅or C₆alkyl), i.e. ester,
(d) phenyl,
(e) naphthyl,
(f) C₃, C₄, C₅or C₆cycloalkyl,
(g) a 5- or 6 membered htereocycle which may be saturated or unsaturated comprising 1, 2, 3, or 4 heteroatoms independently selected from nitrogen , oxygen or sulfur;
(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;
wherein said C₃, C₄, C₅or C₆cycloalkyl, phenyl, naphthyl, are optionally substituted with 1, 2, 3, 4, or 5 substituents independently selected from halogen and R¹⁰²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R¹⁰²⁰; and
(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;
wherein R¹⁰²⁰ is selected from the group consisting of:
(1) hydroxy;
(2) cyano;
(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;
(4) C₁, C₂, C₃, C₄, C₅, C₆, C₇, C₈, C₉ or C₁₀ alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from
(a) hydroxy;
(b) —COOH;
(c) —COO(C₁, C₂, C₃, C₄, C₅or C₆alkyl) i.e. ester, which may linear or branched and is optionally substituted with 1, 2, 3, 4, or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(f) —CONR¹⁰²⁵R¹⁰²⁵;
(g) —SO₂NR¹²⁵R¹²⁵
(h) —NR¹⁰²⁵—C(═O)R¹¹⁰²
(i) —NR¹⁰²⁵—C(═O)NR¹⁰²⁵, R¹⁰²⁵
(j) —NRO25 COOR¹¹³⁰
(k) —O—CO—R¹⁰³⁰
(l) —O—CO—NR¹⁰²⁵, R¹⁰²⁵;
(m) —NR¹⁰²⁵SO₂R¹⁰³⁰;
(n) —NR¹⁰²⁵R¹⁰²⁵;
(o) phenyl which is optionally substituted with 1, 2, 3, 4, or 5 group independently selected from halogen, hydroxy, C₁, C₂, C₃, C₄, Cs or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and
(p) C₃, C₄C₅or C₆cycloalkyl, which is optionally substituted with 1, 2, 3, 4, 5, or 6 halogens;
(5) OC₁, OC₂, OC₃, OC₄, OC₅, OC₆, OC₇, OC₈, OC₉or OC₁₀alkyl, which is linear or branched and is optionally substituted with 0, 1, 2, 3, 4, or 5 halogen atoms and 0, 1, or 2 substitutents selected from
(a) hydroxy;
(b) —COOH;
(c)—COO(C₁, C₂, C₃, C₄, C₅or C₆alkyl) i.e. ester, which may be linear or branched and is optionally substituted with 1, 2, 3, 4 or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(f) —CONR¹⁰²⁵R¹⁰²⁵;
(g) —SO₂NR¹⁰²⁵, R²⁵;
(h) —NR¹⁰²⁵—C(═O)R¹⁰²⁵
(i) —NR¹⁰²⁵C(═O)NR¹⁰²⁵R¹⁰²⁵
(j)—NR¹⁰²⁵ COOR¹³
(k) —O—CO—R¹⁰³⁰
(l)—O—CO_NR¹⁰²⁵R¹⁰²⁵;
(m)—NR¹⁰²⁵SO₂R¹⁰³⁰;
(n) —NR¹⁰²⁵R¹⁰²⁵;
(o) phenyl, which is optionally substituted with 1, 2, 3, 4, or 5 groups independently selected from halogen, hydroxy, C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and
(p) C₃, C₄C₅or C₆cycloalkyl, which is optionally substituted with 1, 2, 3, 4, 5, or 6 halogens;
(6) —COOH;
(7) —COO(C₁, C₂, C₃, C₄, C₅or C₆alkyl) i.e. ester, which may be linear or branched and is optionally substituted with 1, 2, 3, 4, 5 halogens;
(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.
(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(10)—CONR¹⁰²⁵, R¹⁰²⁵;
(11) —SO₂NR¹⁰²⁵R¹⁰²⁵;
(12) —NR¹⁰²⁵—C(═O)R¹¹²⁵
(13)—NR¹¹ ²⁵—C(═O)NR¹⁰²⁵R¹⁰²⁵;
(14) —NR⁹²⁵COOR¹⁰³⁰
(15) —O—CO—R¹⁰³⁰
(16) —O—CO—NR¹R²⁵R¹⁰²⁵;
(17) —NR¹⁰²⁵SO₂R¹⁰³⁰;
(18) —NR¹⁰²⁵R¹⁰²⁵;
(19) phenyl , which is optionally substituted with 1, 2, 3, 4, or 5 group independently selected from halogen, hydroxy, C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;
wherein R¹⁰³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.
wherein R¹⁰²⁵ is selected from R¹⁰³⁰ and hydrogen.
or wherein the group PM
has the formula (XII) 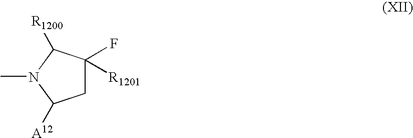
wherein the groups R¹²⁰¹ is hydrogen orfluoro.
wherein R¹²⁰⁰ und A¹² is selected from hydrogen and cyano, and the other is hydrogen.
or wherein the group PM
has the formula XIII: 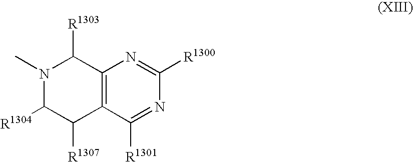
wherein:
R¹³⁰⁰ and RO¹³⁰¹ are independently selected from the group consisting of:
(1) hydrogen,
(2) CN,
(3) C₁₋₁₀alkyl, which is linear or branched which is unsubstituted or substituted with:
a) halogen, or
b) phenyl, which is unsubstituted or substituted with 1-5 substitutents independently selected from halogen, CN, OH, R¹³⁰², OR¹³⁰², NHSO₂R¹³⁰², N(C₁₋₆ alkyl)SOR¹³⁰², SO₂R¹³⁰², SO₂NR¹³⁰⁵R¹³⁰⁶, NR¹³⁰⁵R³⁰⁶, CONR¹¹³⁰⁵R¹³⁰⁶ CO₂H, and CO₂C₁₋₆alkyl, wherein the C₁₋₆alkyl is linear or branched,
(4) phenyl which is unsubstituted or substituted with 1-5 substitutents independently selected from halogen, CN, OH, R¹³⁰², OR¹³⁰², NHSO₂R¹³⁰², N(C₁₋₆alkyl)SO₂R¹³⁰², SO₂R¹³⁰², SO₂NR¹³⁰⁵R¹³⁰⁶, NR¹³⁰⁵R¹³⁰⁶, CONR¹³⁰⁵, R¹³⁰⁶ CO₂H, and CO₂C₁₋₆alkyl, wherein the C₁₋₆alkyl is linear or branched,
(5) a 5- or 6-membered heterocyclic which may be saturated or unsaturated comprising 1-4 heteroatoms independently selected from N, S and O, the heterocycle being unsubstituted or substituted with 1-3 substituents independently selected from oxo, halogen, NO₂, CN, OH, R¹³⁰² OR¹³⁰², NHSO₂R¹³⁰², N(C₁₋₆alkyl)SO₂R¹³⁰², SO₂R¹³⁰², SO₂NR¹³⁰⁵R¹³⁰⁶, NR¹³⁰⁵R¹³⁰⁶, CONR¹³⁰⁵R¹³⁰⁶, CO₂H, and CO₂C₁₋₆alkyl, wherein the C₁₋₆alkyl is linear or branched,
(6) C₃₋₆cycloalkyl, which is optionally substituted with 1-5 substituents independently selected from halogen, OH, C₁₋₆alkyl, and OC₁₋₆alkyl, wherein the C₁₋₆alkyl and OC₁₋₆ alkyl are linear or branched and optionally substituted with 1-5 halogens,
(7) OH,
(8) OR¹³⁰², and
(9) NR¹¹³⁰⁵R¹³⁰⁶
R¹³⁰² is C₁₋₆alkyl, which is linear or branched and which is unsubstituted or substituted with 1-5 groups independently selected from halogen, CO₂H, and CO₂C₁₋₆alkyl, wherein the C₁₋₆alkyl is linear or branched;
R¹³⁰³ is selected from the group consisting of:
(1) hydrogen,
(2) C₁₋₁₀alkyl, which is linear or branched and which is unsubstituted or substituted with one or more substituted selected from:
a) halogen,
b) hydroxy,
c) phenyl, which is unsubstituted or substituted with 1-5 substitutents independently selected from halogen, OH, C₁₋₆alkyl, and OC₁₋₆alkyl, wherein the C₁₋₆alkyl is linear or branched and optionally substituted with 1-5 halogens,
d) naphthyl, wherein the naphthyl is optionally substituted with 1-5 substituents independently selected from halogen, OH, C₁₋₆alkyl, and OC₁₋₆alkyl, wherein the C₁₋₆alkyl is linear or branched and optionally substituted with 1-5 halogens,
a) CO₂H,
b) CO₂C₁₋₆alkyl,
c) CONR¹³⁰⁵R¹³⁰⁶,
(3) CN,
(4) phenyl which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens
(5) naphthyl which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens,
(6) CO₂H,
(7) CO₂C₁₋₆alkyl,
(8) CONR¹³⁰⁵R¹³⁰⁶, and
(9) C₃₋₆cycloalkyl, which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens
—R¹³⁰⁵ and R¹³⁰⁶ are independently selected from the group consisting of:
(1) hydrogen,
(2) phenyl, which is unsubstituted or substituted with substituents independently selected from halogen, OH, C₁₋₆alkyl, and OC₁₋₆alkyl, wherein the C₁₋₆alkyl is linear or branched and optionally substituted with 1-5 halogens,
(3) C₃₋₆cycloalkyl, which is unsubstituted or substituted with 1-5 substituents independently selected from C₁₋₆alkyl, and OC₁₋₆alkyl, wherein the C₁₋₆alkyl is linear or branched and optionally substituted with 1-5 halogens,
(4) C₁₋₆alkyl, which is linear or branched and which is unsubstituted or substituted with:
a) halogen, or
b) phenyl, which is unsubstituted or substituted with 1-5 substituents independently selected from halogen, OH, C₁₋₆alkyl, and OC₁₋₆alkyl, wherein the Cil₆alkyl is linear or branched and optionally substituted with 1-5 halogens,
or wherein R¹³⁰⁵ and R¹³⁰⁶ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆alkyl, and C₁₋₆alkoxy, wherein alkyl and alkoxy are unsubstituted with one to five halogens;
R¹³⁰⁴ and R¹³⁰⁷ are hydrogen;
or wherein the group PM
has the formula XIV: 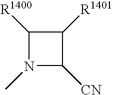
wherein R¹⁴⁰⁰ and R¹⁴⁰¹, independently of each other, are a hydrogen atom (—H); or halogen, cyano or ethynyl;
or wherein the group PM
has the formula XV: 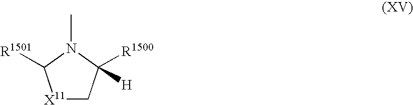
wherein X¹ is CH₂, CHF or CF²;
wherein R¹⁵⁰⁰ is cyano;
wherein R¹⁵⁰¹ is selected from the group consisting of alkoxyalkyl, alkyl, alkylcarbonyl, alkenyl, alkynyl, allenyl, arylalkyl, cycloalkyl, cycloalkylalkyl, cyano, haloalkyl, haloalkenyl, heterocyclealkyl, and hydroxyalkyl;

8. The compound according to claim 1,
wherein the group PM
has the formula (II) 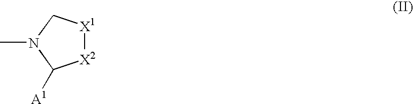
wherein Xi is CR⁵¹R⁵², O, S, or NR⁵³; and
wherein X² is CR⁵⁴R⁵⁵, O, S, or NR⁵⁶; and
wherein R⁵¹, R⁵², R⁵³, R⁵⁴, R⁵⁵, and R⁵⁶, independently of each other, are
a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group or, an amino group (—NH₂), or a N-substituted or N,N-disubstituted amino group (—NHR⁸⁰; —NR⁸¹R⁸²); and
which, independently of each other, can be substituted with one or more substituents, which can be the same or different; and,
wherein optionally, any two of the groups R⁵¹, R⁵², R⁵³, R⁵⁴, R⁵⁵, and R⁵⁶, if present, as well as the pairs R⁶⁶/R⁶⁷, R⁷⁰/R⁷¹, R⁷⁴/R⁷⁵, R⁷⁶/R⁷⁷ and R⁸¹/R⁸², independently of each other, may form a part of a ring; and
wherein the substituents R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³ R⁷⁴, R⁷⁵, R⁷⁶, R⁷⁷, R⁷⁸, R⁷⁹, R⁸⁰, R⁸¹, and R⁸², independently of each other, are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅ cycloalkyl, cyano, amido, thiol trifluoromethyl, or hydroxy group; and
wherein A¹ is
a hydrogen atom (—H) or a carbaldehyde (—CHO), a ketone group (—CO—R¹⁰o), a boronic acid group (—B(OH)₂), a cyano group (—C≡N), a carboxylic acid group (—COOH), a carboxylic acid ester group (—COOR¹⁰¹), a carboxylic acid anhydride group (—CO—O—CO—R¹⁰²), a hydroxamic acid group (—CO—NH(OH)), a N-substituted hydroxamic acid group (—CO—NR¹⁰³ (OH)), a O-substituted hydroxamic acid group (—CO—NH(OR¹⁰⁴)), a carboxamide group (—CO—NH₂), a N-substituted or N,N-disubstituted carboxylic acid amide group, (—CO—NHR¹⁰⁵; —CO—NR¹⁰⁶R¹⁰⁷), an amido group (—HN—CO—R¹⁰⁸), a sulfonic acid group (—SO₃H), a sulfonamide group (—SO₂—NH₂), a N-substituted or N,N-disubstituted sulfonamide group (—SO₂—NHR¹⁰⁹; —SO₂—NR¹¹⁰R¹¹¹.), an amidosulfone group (—NH—SO₂—R¹¹²), a sulfone group (—SO₂—R¹¹³), a phosphoric acid group (—OP(═O)(OH)₂), a phosphoric acid ester group (—OP(═O)(OR¹¹⁴)(OR¹¹⁵)), a phosphonic acid group (—P(═O)(OH)₂), an phosphonic acid ester group (—P(═O)(OR¹¹⁶)(OR¹¹⁷)), a halogen atom, a trifluormethyl group (—CF₃), a thiol group (—SH); a thioether group (—S—R¹¹⁸), a hydroxy group (—OH); an alkoxy group (—O—R¹⁹), a tetrazole group, an amino group (—NH₂), or a N-substituted or N,N-disubstituted amino group (—NHR¹²⁰; —NR¹²¹R¹²²); and wherein optionally, the pairs R¹⁰⁶/R¹⁰⁷, R¹¹⁰/R¹¹¹, R¹¹⁴/R¹¹⁵, R¹¹⁶/R¹¹⁷ and R¹²¹/R¹²², independently of each other, may form a part of a ring; and
which, independently of each other, can be substituted with one or more substituents, which can be the same or different; and,
wherein the substituents R¹⁰⁰, R¹⁰¹, R¹⁰², R¹⁰³, R¹⁰⁴, R¹⁰⁵, R¹⁰⁷, R¹⁰⁸, R¹⁰⁹, R¹¹⁰, R¹¹¹, R¹¹², R¹¹³, R¹¹⁴, R¹¹⁵, R¹¹⁶, R¹¹⁷, R¹¹⁸, R¹¹⁹, R¹²⁰, R¹²¹, and R¹²², independently of each other, are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (III) 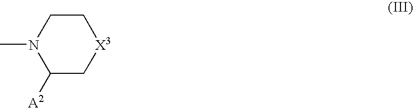
wherein X³ is CR¹³¹R¹³², O, S, or NR¹³³; and
wherein R¹³¹, R¹³², and R¹³³, independently of each other, are
a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group or, an amino group (—NH₂), or a N-substituted or N,N-disubstituted amino group (—NHR¹⁶⁰; —NR¹⁶¹R¹⁶²); and
which, independently of each other, can be substituted with one or more substituents, which can be the same or different; and,
wherein optionally, the the pair R¹³¹/R¹³², if present, as well the pairs R¹⁴⁶/R¹⁴⁷, R¹⁵⁰/R¹⁵¹, R¹⁵⁴/R¹⁵⁵, R¹⁵⁶/R¹⁵⁷ and R¹⁶¹/R¹⁶², independenly of each other, may form a part of a ring; and
wherein the substituents R¹⁴⁰, R¹⁴¹, R¹⁴², R¹⁴³, R¹⁴⁴, R¹⁴⁵, R¹⁴⁶, R¹⁴⁷, R¹⁴⁸, R¹⁴⁹, R¹⁵⁰, R¹⁵¹, R¹⁵², R¹⁵³, R¹⁵⁴, R¹⁵⁵, R¹⁵⁶, R¹⁵⁷, R¹⁵⁸, R¹⁵⁹, R¹⁶⁰, R¹⁶¹, and R¹⁶², independently of each other are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅ branched or straight chain alkenoxy, phenyloxy, benzyloxy, C₃, C₄, C₅cycloalkyl, cyano, amido, thiol trifluoromethyl, or hydroxy group; and
wherein A is
a hydrogen atom (—H); or a carbaldehyde (—CHO), a ketone group (—CO—R¹⁸⁰), a boronic acid group (—B(OH)₂), a cyano group (—C≡N), a carboxylic acid group (—COOH), a carboxylic acid ester group (—COOR⁸), a carboxylic acid anhydride group (—CO—O—CO—R⁸²), a hydroxamic acid group (—CO—NH(OH)), a N-substituted hydroxamic acid group (—CO—NR¹¹⁸³(OH)), a O-substituted hydroxamic acid group (—CO—NH(OR¹⁸⁴)), a carboxamide group (—CO—NH₂), a N-substituted or N,N-disubstituted carboxylic acid amide group, (—CO—NHR¹⁸⁵; —CO—NR¹⁸⁶R¹⁸⁷), an amido group (—HN—CO—R¹⁸⁸), a sulfonic acid group (—SO₃H), a sulfonamide group (—SO₂—NH₂), a N-substituted or N,N-disubstituted sulfonamide group (—SO₂—NHR¹⁸⁹; —SO₂—NR¹⁹° R¹⁹¹), an amidosulfone group (—NH—SO₂—R¹²), a sulfone group (—SO₂—R¹⁹³), a phosphoric acid group (—OP(═O)(OH)₂), a phosphoric acid ester group (—OP(═O)(OR¹⁹⁴)(OR¹⁹⁵)), a phosphonic acid group (—P(═O)(OH)₂), an phosphonic acid ester group (—P(═O)(OR¹⁹⁶)(OR¹⁹⁷)), a halogen atom, a trifluormethyl group (—CF₃), a thiol group (—SH); a thioether group (—S—R¹⁹⁸), a hydroxy group (—OH); an alkoxy group (—O—R¹⁹⁹), a tetrazole group, an amino group (—NH₂), or a N-substituted or N,N-disubstituted amino group (—NHR²⁰⁰; —NR²⁰¹R²⁰²); and
which, independently of each other, can be substituted with one or more substituents, which can be the same or different; and,
wherein optionally, the pairs R¹⁸⁶R¹⁸⁷, R¹⁹⁰/R¹⁹¹, R¹⁹⁴/R¹⁹⁵, R¹⁹⁶/R¹⁹⁷ and R²⁰¹/R²⁰² independenly of each other, may form a part of a ring; and
wherein the substituents R¹⁸⁰, R¹⁸¹, R¹⁸², R¹⁸³, R¹⁸⁴, R¹⁸⁵, R¹⁸⁶, R¹⁸⁷, R¹⁸⁸, R¹⁸⁹, R¹⁹⁰, R¹⁹¹, R¹⁹², R¹⁹³, R¹⁹⁴, R¹⁹⁵, R¹⁹⁶, R¹⁹⁷, R¹⁹⁸, R¹⁹⁹, R²⁰⁰, R²⁰¹, and R²⁰², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (IV) 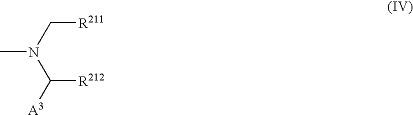
wherein R²¹¹ and R²¹², independently of each other, are
a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group or, an amino group (—NH₂), or a N-substituted or N,N-disubstituted amino group (—NHR²⁴⁰; —NR²⁴R²⁴²); and
which, independently of each other, can be substituted with one or more substituents, which can be the same or different; and,
wherein optionally, the pair R²¹¹/R²¹², as well the pairs R²²⁶/R²²⁷, R²³⁰/R²³¹, R²³⁴/R²³⁵, R²³⁶/R²³⁷ and R²⁴¹/R²⁴², independenly of each other, may form a part of a ring; and
wherein the substituents R²²⁰, R²²¹, R²²², R²²³, R²²⁴, R²²⁵, R²²⁶, R²²⁷, R²²⁸, R²²⁹, R²³⁰, R²³¹, R²³², R²³³, R²³⁴, R²³⁵, R²³⁶, R²³⁷, R²³⁸, R²³⁹, R²⁴⁰, R²⁴¹, and R²⁴², independently of each other, are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅cycloalkyl, cyano, amido, thiol trifluoromethyl, or hydroxy group; and
wherein A³ is
a hydrogen atom (—H); or a carbaldehyde (—CHO), a ketone group (—CO—R²⁶⁰), a boronic acid group (—B(OH)₂), a cyano group (—C≡N), a carboxylic acid group (—COOH), a carboxylic acid ester group (—COOR²⁶¹), a carboxylic acid anhydride group (—CO—O—CO—R²⁶²), a hydroxamic acid group (—CO—NH(OH)), a N-substituted hydroxamic acid group (—CO—NR²⁶³(OH)), a O-substituted hydroxamic acid group (—CO—NH(OR²⁶⁴)), a carboxamide group (—CO—NH₂), a N-substituted or N,N-disubstituted carboxylic acid amide group, (—CO—NHR²⁶⁵; —CO—NR²⁶⁶, R²⁶⁷), an amido group (—HN—CO—R²⁶⁸), a sulfonic acid group (—SO₃H), a sulfonamide group (—SO₂—NH₂), a N-substituted or N,N-disubstituted sulfonamide group (—SO₂—NHR²⁶; —SO₂—NR²⁷⁰R²⁷¹), an amidosulfone group (—NH—SO₂—R²⁷²), a sulfone group (—SO₂—R²⁷), a phosphoric acid group (—OP(═O)(OH)₂), a phosphoric acid ester group (—OP(═O)(OR²⁷⁴)(OR²⁷⁵)), a phosphonic acid group (—P(═O)(OH)₂), an phosphonic acid ester group (—P(═O)(OR²⁷⁶)(OR²⁷⁷)), a halogen atom, a trifluormethyl group (—CF₃), a thiol group (—SH); a thioether group (—S—R²⁷⁸), a hydroxy group (—OH); an alkoxy group (—O—R²⁷⁹), a tetrazole group, an amino group (—NH₂), or a N-substituted or N,N-disubstituted amino group (—NHR²⁸⁰; —NR²⁸¹R²⁸²); and
which, independently of each other, can be substituted with one or more substituents, which can be the same or different; and,
wherein optionally, the pairs R²⁶⁶/R²⁶⁷, R²⁷⁰/R²⁷¹, R²⁷⁴/R²⁷⁵, R²⁷⁶/R²⁷⁷ and R²⁸¹/R²⁸², independenly of each other, may form a part of a ring; and
wherein the substituents R²⁶⁰, R²⁶¹, R²⁶², R²⁶³, R²⁶⁴, R²⁶⁵, R²⁶⁶, R²⁶⁷, R²⁶⁸, R²⁶⁹, R²⁷⁰, R²⁷¹, R²⁷², R²⁷³, R²⁷⁴, R²⁷⁵, R²⁷⁶, R²⁷⁷, R²⁷⁸, R²⁷⁹, R²⁸⁰, R²⁸¹, and R²⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM (p) C₃, C₄C₅or C₆cycloalkyl, which is optionally substituted with 1, 2, 3, 4, 5, or 6 halogens;
(6) —COOH;
(7) —COO(C₁, C₂, C₃, C₄, C₅or C₆alkyl) i.e. ester, which may be linear or branched and is optionally substituted with 1, 2, 3, 4, 5 halogens;
(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.
(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(10) —CONR⁹²⁵R⁹²⁵;
(11) —SO₂NR⁹²⁵R⁹²⁵;
(12) —NR⁹²⁵—C(═O)R⁹²¹
(13) —NR⁹²⁵—C(═O)NR⁹²⁵R⁹²⁵;
(14) —NR⁹²⁵COOR⁹³⁰
(15)—O—CO—R⁹³⁰
(16) —O—CO—NR⁹²⁵, R⁹²;
(17) —NR⁹²⁵SO R⁹³⁰;
(18) —NR⁹²⁵R⁹²⁵;
(19) phenyl , which is optionally substituted with 1, 2, 3, 4, or 5 group independently selected from halogen, hydroxy, C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;
wherein R⁹³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.
wherein R⁹²⁵ is selected from R⁹³⁰ and hydrogen.
wherein the group PM
has the formula (XI) 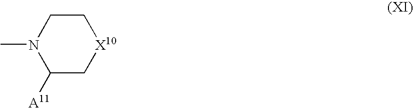
wherein the groups X¹⁰ is CR¹⁰⁰⁰ R¹⁰⁰¹, S, SO, SO₂or NR¹⁰⁰²
wherein R¹⁰⁰⁰, R¹⁰⁰¹ and R¹⁰⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR¹⁰¹⁰R¹⁰¹¹.
and A¹¹ is selected from hydrogen, cyano, —C(═O)NR¹⁰¹²R¹⁰¹³, or C₁, C₂, C₃, C₄, C₅or C₆alkyl, which is linear or branched and is optionally substituted with 1, 2, 3, 4, or 5 halogens;
wherein
—R¹⁰¹⁰ and R¹⁰¹², are, independently of each other, selected from hydrogen, or C₁, C₂, C₃, C₄, C₅or C₆alkyl, which is linear or branched and is optionally substituted with 1, 2, 3, 4, or 5 halogens; and
R¹⁰¹¹ and R¹⁰¹³, are, independently of each other, selected from the group consisting of
(1) phenyl, which is optionally substituted with 1, 2, 3, 4, or 5, substituents independently selected from halogen and R¹⁰²⁰;
(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of
(a) hydroxy,
(b) —COOH,
(c) —COO(C₁, C₂, C₃, C₄, C₅or C₆alkyl), i.e. ester,
(d) phenyl,
(e) naphthyl,
(f) C₃, C₄, C₅or C₆cycloalkyl,
(g) a 5- or 6 membered htereocycle which may be saturated or unsaturated comprising 1, 2, 3, or 4 heteroatoms independently selected from nitrogen , oxygen or sulfur;
(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;
wherein said C₃, C₄, C₅or C₆cycloalkyl, phenyl, naphthyl, are optionally substituted with 1, 2, 3, 4, or 5 substituents independently selected from halogen and R¹⁰²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R⁰²¹; and
(3) C₃, C₄CS or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;
wherein R¹⁰²⁰ is selected from the group consisting of:
(1) hydroxy;
(2) cyano;
(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;
(4) C₁, C₂, C₃, C₄, C₅, C₆, C₇, C₈, C₉or C₁₀alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from
(a) hydroxy;
(b) —COOH;
(c) —COO(C₁, C₂, C₃, C₄, C₅or C₆alkyl) i.e. ester, which may linear or branched and is optionally substituted with 1, 2, 3, 4, or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OCs or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(f) —CONR¹⁰²⁵, R¹²⁵;
(g) —SO₂NR¹⁰²⁵R¹⁰²⁵;
(h) —NR²¹—C(═O)R¹²⁵
(i) —NR¹⁰²⁵—C(═O)NR¹⁰²⁵, R¹²⁵
(j) —NR¹⁰²⁵ COOR¹⁰³⁰
(k) —O—CO—R¹⁰³⁰
(l) —O—CO—NR¹⁰²⁵R¹⁰²⁵;
(m) —NR¹⁰²⁵ SR¹⁰³⁰;
(n) —NR¹⁰²⁵R¹⁰²⁵;
(o) phenyl which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and
(p) C₃, C₄C₅or C₆cycloalkyl, which is optionally substituted with 1, 2, 3, 4, 5, or 6 halogens;
(5) OC₁, OC₂, OC₃, OC₄, OC₅, OC₆, OC₇, OC₈, OC₉or OC₁₀alkyl, which is linear or branched and is optionally substituted with 0, 1, 2, 3, 4, or 5 halogen atoms and 0, 1, or 2 substitutents selected from
(a) hydroxy;
(b) —COOH;
(c) —COO(C₁, C₂, C₃, C₄, C₅or C₆alkyl) i.e. ester, which may be linear or branched and is optionally substituted with 1, 2, 3, 4 or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(f) —CONR¹⁰²⁵, R¹⁰²⁵;
(g) —SO₂NR¹⁰²⁵R¹⁰²⁵;
(h) —NR¹⁰²⁵—C(═O)R¹⁰²⁵
(i) —NR¹⁰²⁵—C(═O)NR¹⁰²⁵, R¹²⁵
(j)—NR¹⁰²⁵COOR¹⁰³⁰
(k) —O—CO—R¹⁰³⁰
(l) —O—CO—NR¹⁰²⁵R¹⁰²⁵;
(m) —NR¹⁰²⁵SO₂R¹⁰³⁰;
(n) —NR¹⁰²⁵R¹⁰²⁵;
(o) phenyl, which is optionally substituted with 1, 2, 3, 4, or 5 groups independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and
(p) C₃, C₄C₅or C₆cycloalkyl, which is optionally substituted with 1, 2, 3, 4, 5, or 6 halogens;
(6) —COOH;
(7)—COO(C₁, C₂, C₃, C₄, C₅or C₆alkyl) i.e. ester, which may be linear or branched and is optionally substituted with 1, 2, 3, 4, 5 halogens;
(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.
(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(10) —CONR¹⁰²⁵R¹⁰²⁵;
(11) —SO₂NR¹⁰²⁵, R¹⁰²⁵;
(12) —NR¹⁰²⁵—C(═O)R¹¹²⁵
(13) —NR¹¹⁰²⁵—C(═O)NR¹⁰²⁵R¹⁰²⁵;
(14) —NR⁹²⁵COOR¹⁰³⁰
(15) —O—CO—R¹⁰³⁰
(16)—O—CO—NR¹⁰²⁵R¹⁰²⁵;
(17) —NR¹⁰²⁵SO₂R¹⁰³⁰;
(18)—NR¹R²⁵R¹⁰²⁵;
(19) phenyl , which is optionally substituted with 1, 2, 3, 4, or 5 group independently selected from halogen, hydroxy, C₁, C₂, C₃, C₄, Cs or C₆alkyl, —OC₁, —OC₂, —OC₃, —OC₄, —OC₅ or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;
wherein R¹⁰³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.
wherein R¹⁰²⁵ is selected from R¹⁰³⁰ and hydrogen.
or wherein the group PM
has the formula (XII) 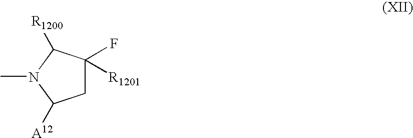
wherein the groups R¹²⁰¹ is hydrogen orfluoro.
wherein R¹²⁰⁰ und A¹² is selected from hydrogen and cyano, and the other is hydrogen.
or wherein the group PM
has the formula XIII: 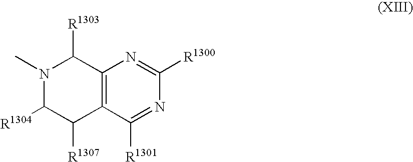
wherein:
R¹³⁰⁰ is selected from the group consisting of:
(1) hydrogen,
(2) CN,
(3) C₁₋₁₀alkyl, which is linear or branched and which is unsubstituted or substituted with:
a) halogen, or
b) phenyl, which is unsubstituted or substituted with 1-5 substitutents independently selected from halogen, CN, OH, R¹³⁰², OR¹³⁰², NHSO₂R¹³⁰², N(C₁₋₆alkyl)SO₂R¹³⁰²SO₂R¹³⁰², SO₂NR¹³⁰⁵R¹³⁰⁶ NR¹³⁰⁵R¹³⁰⁶, CONR¹³⁰⁵R¹³⁰⁶, CO₂H, and CO₂C₁₋₆alkyl, wherein the C₁₋₆alkyl is linear or branched,
(4) phenyl which is unsubstituted or substituted with 1-5 substitutents independently selected from halogen, CN, OH, R¹³⁰², OR¹³⁰², NHSO₂R¹³⁰², N(C₁₋₆alkyl)SO₂R¹³⁰², SO₂R¹³⁰², SO₂NR¹³⁰⁵R¹³⁰⁶, NR¹³⁰⁵R¹³⁰⁶, CONR¹³⁰⁵R¹³⁰⁶ CO₂H, and CO₂CO₁₋₆ and wherein the C₁₋₆alkyl is linear or branched,
(5) a 5- or 6-membered heterocyclic which may be saturated or unsaturated comprising 1-4 heteroatoms independently selected from N, S and O, the heterocycle being unsubstituted or substituted with 1-3 substituents independently selected from oxo, halogen, NO₂, CN, OH, R¹³⁰², OR¹³⁰², NHSO₂R¹³⁰², N(C₁₋₆alkyl)SO₂R¹³⁰², SO₂R¹³⁰², SO₂NR¹³⁰⁵R¹³⁰⁶, NR¹³⁰⁵R¹³⁰⁶, CONR¹³⁰⁵, R¹³⁰⁶, CO₂H, and CO₂C₁₋₆alkyl, wherein the C₁₋₆alkyl is linear or branched,
(6) C₃₋₆cycloalkyl, which is optionally substituted with 1-5 substituents independently selected from halogen, OH, C₁₋₆alkyl, and OC₁₋₆alkyl, wherein the C₁₋₆alkyl and OC₁₋₆ alkyl are linear or branched and optionally substituted with 1-5 halogens
(7) OH
(8) OR¹³⁰² and
(9) NR¹³⁰⁵R¹³⁰⁶;
and R¹³⁰¹ is hydrogen;
R¹³⁰² is C₁₋₆alkyl, which is linear or branched and which is unsubstituted or substituted with 1-5 groups independently selected from halogen, CO₂H, and CO₂C₁₋₆alkyl, wherein the C₁₋₆alkyl is linear or branched;
R¹³⁰³ is selected from the group consisting of:
(1) hydrogen,
(2) C₁₋₁₀alkyl, which is linear or branched and which is unsubstituted or substituted with one or more substituted selected from:
a) halogen,
b) hydroxy,
c) phenyl, which is unsubstituted or substituted with 1-5 substitutents independently selected from halogen, OH, C₁₋₆alkyl, and OC₁₋₆alkyl, wherein the C₁₋₆alkyl is linear or branched and optionally substituted with 1-5 halogens,
d) naphthyl, wherein the naphthyl is optionally substituted with 1-5 substituents independently selected from halogen, OH, C₁₋₆alkyl, and OC₁₋₆alkyl, wherein the C₁₋₆alkyl is linear or branched and optionally substituted with 1-5 halogens,
e) CO₂H,
f) CO₂C₁₋₆alkyl,
g) CONR¹³⁰⁵, R¹³⁰⁶
(3) CN,
(4) phenyl which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens,
(5) naphthyl which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens,
(6) CO₂H,
(7) CO₂C₁₋₆alkyl,
(8) CONR¹³⁰⁵, R¹³⁰⁶, and
(9) C₃₋₆cycloalkyl, which is unsubstituted or substituted with 1-5 substituents independently selected from C₁₋₆alkyl, and OC₁₋₆alkyl, hydroxy and halogen, wherein the C₁₋₆alkyl is linear or branched and optionally substituted with 1-5 halogens
R¹³⁰⁵ and R¹³⁰⁶ are independently selected from the group consisting of:
(1) hydrogen,
(2) phenyl, which is unsubstituted or substituted with substituents independently selected from halogen, OH, C₁₋₆alkyl, and OC₁₋₆alkyl, wherein the C₁₋₆alkyl is linear or branched and optionally substituted with 1-5 halogens
(3) C₃₋₆cycloalkyl, which is unsubstituted or substituted with 1-5 substituents independently selected from C₁₋₆alkyl, and OC₁₋₆alkyl, wherein the C₁₋₆alkyl is linear or branched and optionally substituted with 1-5 halogens
(4) C₁₋₆alkyl, which is linear or branched and which is unsubstituted or substituted with:
a) halogen, or
b) phenyl, which is unsubstituted or substituted with 1-5 substituents independently selected from halogen, OH, C₁₋₆alkyl, and OC₁₋₆alkyl, wherein the C₁₋₆alkyl is linear or branched and optionally substituted with 1-5 halogens,
or wherein R¹³⁰⁵ and R¹³⁰⁶ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆alkyl, and C₁₋₆alkoxy, wherein alkyl and alkoxy are unsubstituted with one to five halogens;
R¹³⁰⁴ and R¹³⁰⁷ are hydrogen;
or wherein the group PM
has the formula XIV: 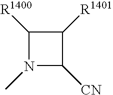
has the formula (V) 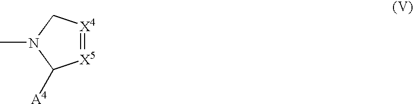
wherein X⁴ is CR²⁹¹ or N; and
wherein X is CR²⁹² or N; and
wherein R²⁹¹ and R²⁹², independently of each other, are
a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group or an amino group (—NH₂), or a N-substituted or N,N-disubstituted amino group (—NHR³²⁰; —NR³²¹R³²²); and
which, independently of each other, can be substituted with one or more substituents, which can be the same or different; and,
wherein optionally, the the pair R²⁹¹/R²⁹², if present, as well the pairs R³⁰⁶/R³⁰⁷, R³¹⁰/R³¹¹, R³¹⁴/R³¹⁵, R³¹⁶/R³¹⁷ and R³²¹/R³²², independenly of each other, may form a part of a ring; and
wherein the substituents R³⁰⁰, R³⁰¹, R³⁰², R³⁰³, R³⁰⁴, R³⁰⁵, R³⁰⁶, R³⁰⁷, R³⁰⁸, R³⁰⁹, R³¹⁰, R³¹¹, R³¹², R³¹³, R³¹⁴, R³¹⁵, R³¹⁶, R³¹⁷, R³¹⁷, R³¹⁸, R³¹⁹, R³²⁰ and R³²¹, independently of each other are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅cycloalkyl, cyano, amido, thiol trifluoromethyl, or hydroxy group; and
wherein A⁴ is
a hydrogen atom (—H); or a carbaldehyde (—CHO), a ketone group (—CO—R³⁴⁰), a boronic acid group (—B(OH)₂), a cyano group (—C≡N), a carboxylic acid group (—COOH), a carboxylic acid ester group (—COOR³⁴¹), a carboxylic acid anhydride group (—CO—O—CO—R³⁴²), a hydroxamic acid group (—CO—NH(OH)), a N-substituted hydroxamic acid group (—CO—NR³⁴³(OH)), a O-substituted hydroxamic acid group (—CO—NH(OR³⁴⁴)), a carboxamide group (—CO—NH₂), a N-substituted or N,N-disubstituted carboxylic acid amide group, (—CO—NHR³⁴⁵; —CO—NR³⁴⁶R³⁴⁷), an amido group (—HN—CO—R³⁴⁸), a sulfonic acid group (—SO₃H), a sulfonamide group (—SO₂—NH₂), a N-substituted or N,N-disubstituted sulfonamide group (—SO₂—NHR³⁴⁹; —SO₂—NR³⁵RR³⁵¹), an amidosulfone group (—NH—SO₂—R³⁵²), a sulfone group (—SO₂—R³³), a phosphoric acid group (—OP(═O)(OH)₂), a phosphoric acid ester group (—OP(═O)(OR³⁵⁴)(OR³⁵⁵)), a phosphonic acid group (—P(═O)(OH)₂), an phosphonic acid ester group (—P(═O)(OR³⁵⁶)(OR³⁵⁷.)), a halogen atom, a trifluormethyl group (—CF₃), a thiol group (—SH); a thioether group (—S—R³⁵⁸), a hydroxy group (—OH); an alkoxy group (—O—R³⁵⁹), a tetrazole group, an amino group (—NH₂), or a N-substituted or N,N-disubstituted amino group (—NHR³⁶⁰; —NR³⁶¹R³⁶²); and
which, independently of each other, can be substituted with one or more substituents, which can be the same or different; and,
wherein optionally, the pairs R³⁴⁶/R³⁴⁷, R³⁵⁰/R³⁵¹, R³⁵⁴/R³⁵⁵, R³⁵⁶/R³⁵⁷ and R³⁶¹/R³⁶² independenly of each other, may form a part of a ring; and
wherein the substituents R³⁴⁰, R³⁴¹, R³⁴², R³⁴³, R³⁴⁴, R³⁴⁵, R³⁴⁶, R³⁴⁷, R³⁴⁸, R³⁴⁹, R³⁵⁰, R³⁵¹, R³⁵², R³⁵³, R³⁵⁴, R³⁵⁵, R³⁵⁶, R³⁵⁷, R³⁵⁸, R³⁵⁹, R³⁶⁰, R³⁶¹ and R³⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (VI) 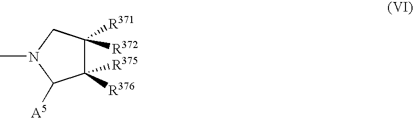
wherein R³⁷¹, R³⁷², R³⁷⁵ and R³⁷⁶, independently of each other, a hydrogen atom (—H); or a C₁, C₂, C₃, C₄, and C₅ branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, and aryl, heteroaryl, aryl-alkyl, heteroaryl-alkyl, aryl-heteroalkyl, heteroaryl-heteroalkyl group or, a carbaldehyde (—CHO), a ketone group (—CO—R³⁸⁰), a boronic acid group (—B(OH)₂), a cyano group (—C≡N), a carboxylic acid group (—COOH), a carboxylic acid ester group (—COOR³⁸⁰), a halogen atom, a trifluormethyl group (—CF₃), a thiol group (—SH); a thioether group (—S—R³⁹⁸), a hydroxy group (—OH); an alkoxy group (—O—R³⁹⁹), a tetrazole group, an amino group (—NH₂), or a N-substituted or N,N-disubstituted amino group (—NHR⁴⁰⁰; —NR⁴⁰¹R⁴⁰²); and
which, independently of each other, can be substituted with one or more substituents, which can be the same or different; and,
wherein optionally, any two of the groups R³⁷¹, R³⁷², R³⁷⁵, and R³⁷⁶, as well as the pairs R³⁸⁶/R³⁸⁷, R³⁹⁰/R³⁹¹, R³⁹⁴/R³⁹⁵, R³⁹⁶/R³⁹⁷ and R⁴⁰¹/R⁴⁰², independenly of each other, may form a part of a ring; and
wherein the substituents R³⁸⁰, R³, R³⁸², R³⁸³, R³⁸⁴, R³⁸⁵, R³⁸⁶, R³⁷, R³⁸, R³⁸⁹, R³⁹⁰ R³⁹¹, R³⁹², R³⁹³, R³⁹⁴, R³⁹⁵, R³⁹⁶, R³⁹⁷, R³⁹⁸, R³⁹⁹, R⁴⁰⁰, R⁴⁰¹, and R⁴⁰², independently of each other are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅cycloalkyl, cyano, amido, thiol trifluoromethyl, or hydroxy group; and
alternatively; the two groups R³⁷¹ and R³⁷² can be together an oxo (═O) or hydroxyimino (═N—OH) group; and
alternatively; the two groups R³⁷⁵ and R³⁷⁶ can be together an oxo (═O) or hydroxyimino (═N—OH) group; and
wherein A⁵ is
a hydrogen atom (—H); or a carbaldehyde (—CHO), a ketone group (—CO—R⁴²⁰), a boronic acid group (—B(OH)₂), a cyano group (—C≡N), a carboxylic acid group (—COOH), a carboxylic acid ester group (—COOR⁴²¹), a carboxylic acid anhydride group (—CO—O—CO—R⁴²²), a hydroxamic acid group (—CO—NH(OH)), a N-substituted hydroxamic acid group (—CO—NR⁴²³(OH)), a O-substituted hydroxamic acid group (—CO—NH(OR⁴²⁴)), a carboxamide group (—CO—NH₂), a N-substituted or N,N-disubstituted carboxylic acid amide group, (—CO—NHR⁴²⁵; —CO—NR⁴²⁶, R⁴²⁷), an amido group (—HN—CO—R⁴²⁸), a sulfonic acid group (—SO₃H), a sulfonamide group (—SO₂—NH₂), a N-substituted or N,N-disubstituted sulfonamide group (—SO₂—NHR⁴²⁹; —SO₂—NR⁴³⁰R⁴³¹), an amidosulfone group (—NH—SO₂—R⁴³²), a sulfone group (—SO₂—R⁴³³), a phosphoric acid group (—OP(═O)(OH)₂), a phosphoric acid ester group (—OP(═O)(OR⁴³⁴)(OR⁴³⁵)), a phosphonic acid group (—P(═O)(OH)₂), an phosphonic acid ester group (—P(═O)(OR⁴³⁶)(OR⁴³⁷)), a halogen atom, a trifluormethyl group (—CF₃), a thiol group (—SH); a thioether group (—S—R⁴³⁸), a hydroxy group (—OH); an alkoxy group (—O—R⁴³⁹), a tetrazole group, an amino group (—NH₂), or a N-substituted or N,N-disubstituted amino group (—NHR⁴⁴⁰; —NR⁴⁴R⁴⁴²); and
which, independently of each other, can be substituted with one or more substituents, which can be the same or different; and,
wherein optionally, the pairs R⁴²⁶/R⁴²⁷, R⁴³⁰/R⁴³¹, R⁴³⁴/R⁴³⁵, R⁴³⁶/R⁴³⁷ and R⁴⁴¹/R⁴⁴² independenly of each other, may form a part of a ring; and
wherein the substituents R⁴²⁰, R⁴²¹, R⁴²², R⁴²³, R⁴²⁴, R⁴², R⁴⁶, R⁴²⁷, R⁴²⁸, R⁴²⁹, R⁴³⁰ R⁴³¹, R⁴³², R⁴³³, R⁴³⁴, R⁴³⁵, R⁴³⁶, R⁴³⁷, R⁴³⁸, R⁴³⁹, R⁴⁴⁰, R⁴⁴¹, and R⁴⁴², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (VII) 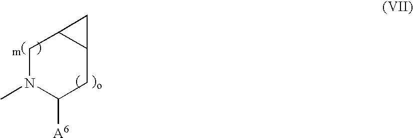
wherein m is equal to 1 or 2, and o is equal to 1 or 2, and m or o can be equal to 0;
wherein A⁶ is a hydrogen atom (—H); or a carbaldehyde (—CHO), a ketone group (—CO—R⁴⁶⁰), a boronic acid group (—B(OH)₂), a cyano group (—C≡N), a carboxylic acid group (—COOH), a carboxylic acid ester group (—COOR⁴⁶¹), a carboxylic acid anhydride group (—CO—O—CO—R⁴⁶²), a hydroxamic acid group (—CO—NH(OH)), a N-substituted hydroxamic acid group (—CO—NR⁴⁶³ (OH)), a O-substituted hydroxamic acid group (—CO—NH(OR⁴⁶⁴)), a carboxamiide group (—CO—NH₂), a N-substituted or N,N-disubstituted carboxylic acid amide group, (—CO—NHR⁴⁶⁵; —CO—NR⁴⁶⁶R⁴⁶⁷), an amido group (—HN—CO—R⁴⁶⁸), a sulfonic acid group (—SO₃H), a sulfonamide group (—SO₂—NH₂), a N-substituted or N,N-disubstituted sulfonamide group (—SO₂—NHR⁴⁶⁹; —SO₂—NR⁴⁷⁰R⁴⁷¹), an amidosulfone group (—NH—SO₂—R⁴⁷²), a sulfone group (—SO₂—R⁴⁷³), a phosphoric acid group (—OP(═O)(OH)₂), a phosphoric acid ester group (—OP(═O)(OR⁴⁷⁴)(OR⁴⁷⁵)), a phosphonic acid group (—P(═O)(OH)₂), an phosphonic acid ester group (—P(═O)(OR⁴⁷⁶)(OR⁴⁷⁷)), a halogen atom, a trifluormethyl group (—CF₃), a thiol group (—SH); a thioether group (—S—R⁴⁷⁸), a hydroxy group (—OH); an alkoxy group (—O—R⁴⁷⁹), a tetrazole group, an amino group (—NH₂), or a N-substituted or N,N-disubstituted amino group (—NHR⁴⁸⁰; —NR⁴⁸¹R⁴⁸²);
which, independently of each other, can be substituted with one or more substituents, which can be the same or different; and,
wherein optionally, the pairs R⁴⁶⁶/R⁴⁶⁷, R⁴⁷⁰/R⁴⁷¹, R⁴⁷⁴/R⁴⁷⁵, R⁴⁷⁶/R⁴⁷⁷ and R⁴⁸¹/R⁴⁸² independenly of each other, may form a part of a ring; and
wherein the substituents R⁴⁶⁰, R⁴⁶¹, R⁴⁶², R⁴⁶³, R⁴⁶⁴, R⁴⁶⁵, R⁴⁶⁶, R⁴⁶⁷, R⁴⁶⁸, R⁴⁶⁹, R⁴⁷⁰R⁴⁷¹, R⁴⁷², R⁴⁷³, R⁴⁷⁴, R⁴⁷⁵, R⁴⁷⁶, R⁴⁷⁷, R⁴⁷⁸, R⁴⁷⁹, R⁴⁸⁰, R⁴⁸¹, and R⁴⁸², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (VIII) 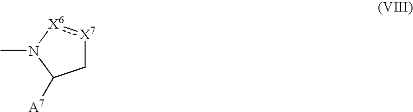
wherein X⁶ is selected from CR⁴⁹⁰R⁴⁹¹, O, S or NR⁴⁹², when the bond between X⁶ and X⁷ is a single bond; and
wherein X⁷ is selected from CR⁴⁹³R⁴⁹⁴, O, S, or NR⁴⁹⁵, when the bond between X⁶ and X⁷ is a single bond;
or alternatively,
wherein X⁶ is selected from CR⁴⁹⁶ or N, when the bond between X⁶ and X⁷ is a double bond; and
wherein X⁷ is selected from CR⁴⁹⁷ or N, when the bond between X⁶ and X⁷ is a double bond; and
wherein R⁴⁹⁰, R^{491 , R}⁴⁹², R⁴⁹³, R⁴⁹⁴, R⁴⁹⁵, R⁴⁹⁶ and R⁴⁹⁷, independently of each other, are a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group, or an amino group (—NH₂), or a N-substituted or N,N-disubstituted amino group (—NHR¹; —NR⁵²¹, R⁵²²); and
which, independently of each other, can be substituted with one or more substituents, which can be the same or different; and,
wherein optionally, any two the groups R⁴⁹⁰, R⁴⁹¹, R², R⁴⁹³, R⁴⁹⁴, R⁴⁹⁵ R⁴⁹⁶, and R⁴⁹⁷, if present, as well as the pairs R⁵⁰⁶/R⁵⁰⁷, R⁵¹⁰/R⁵¹¹, R⁵¹⁴/R⁵¹⁵, R⁵¹⁶/R⁵¹⁷ and R⁵²¹/R⁵²² independenly of each other, may form a part of a ring; and
wherein the substituents R⁵⁰⁰, R⁵⁰¹, R⁵⁰², R⁵⁰³, R⁵⁵⁰⁴, R⁵⁰⁵, R⁵⁰⁶, R⁵⁰⁷, R⁵⁰⁸, R⁵⁰⁹, R⁵¹⁰, R⁵¹¹, R⁵¹², R⁵¹³, R⁵¹⁴, R⁵¹⁵, R⁵¹⁶, R⁵¹⁷, R⁵¹⁸, R⁵¹⁹, R⁵²⁰, R⁵²¹, and R⁵²², independently of each other are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅ cycloalkyl, cyano, amido, thiol trifluoromethyl, or hydroxy group; and
wherein A⁷ is
a hydrogen atom (—H); or a carbaldehyde (—CHO), a ketone group (—CO—R⁵⁴⁰), a boronic acid group (—B(OH)₂), a cyano group (—C≡N), a carboxylic acid group (—COOH), a carboxylic acid ester group (—COOR⁵⁴¹), a carboxylic acid anhydride group (—CO—O—CO—R⁴²), a hydroxamic acid group (—CO—NH(OH)), a N-substituted hydroxamic acid group (—CO—NR⁵⁴³ (OH)), a O-substituted hydroxamic acid group (—CO—NH(OR⁵⁴⁴)), a carboxamide group (—CO—NH₂), a N-substituted or N,N-disubstituted carboxylic acid amide group, (—CO—NHR⁵⁴⁵; —CO—NR⁵⁴⁶R⁵⁴⁷), an amido group (—HN—CO—R⁵⁴⁸), a sulfonic acid group (—SO₃H), a sulfonamide group (—SO₂—NH₂), a N-substituted or N,N-disubstituted sulfonamide group (—SO₂—NHR⁵⁴⁹;
—SO₂—NR⁵¹R⁵⁵¹), an amidosulfone group (—NH—SO₂—R⁵⁵²), a sulfone group (—SO₂—R⁵⁵³), a phosphoric acid group (—OP(═O)(OH)₂), a phosphoric acid ester group (—OP(═O)(OR⁵⁵⁴)(OR⁵⁵⁵)), a phosphonic acid group (—P(═O)(OH)₂), an phosphonic acid ester group (—P(═O)(OR⁵⁵⁶)(OR⁵⁵⁷)), a halogen atom, a trifluormethyl group (—CF₃), a thiol group (—SH); a thioether group (—S—R⁵⁵⁸), a hydroxy group (—OH); an alkoxy group (—O—R⁵⁵⁹), a tetrazole group, an amino group (—NH₂), or a N-substituted or N,N-disubstituted amino group (—NHR⁵⁶⁰; —NR⁵⁶¹R⁵⁶²); and
which, independently of each other, can be substituted with one or more substituents, which can be the same or different; and,
wherein optionally, the pairs R⁵⁴⁶/R⁵⁴⁷, R⁵⁵⁰/R⁵⁵¹, R⁵⁵⁴/R⁵⁵⁵, R⁵⁵⁶/R⁵⁵⁷ and R⁵⁶¹/R⁵⁶² independenly of each other, may form a part of a ring; and
wherein the substituents R⁵⁴⁰, R⁵⁴¹, R⁵⁴², R⁵⁴³, R⁵⁴⁴, R⁵⁴⁵, R⁵⁴⁶⁵⁴⁷, R⁵⁴⁸, R⁵⁴⁹, R⁵⁵⁰ R⁵⁵¹, R⁵⁵², R⁵⁵³, R⁵⁵⁴, R⁵⁵⁵, R⁵⁵⁶, R⁵⁵⁷, R⁵⁵⁸, R⁵⁵⁹, R⁵⁶⁰, R⁵⁶¹, and R⁵⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (IX) or (IXa) 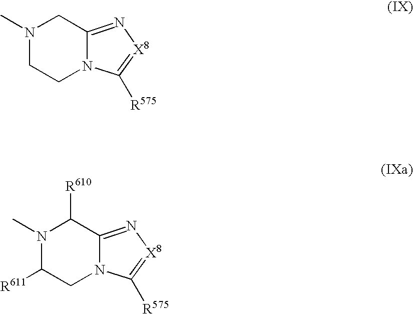
wherein X⁸ is N or CR⁵⁷⁰; and
wherein R⁵⁷⁰, R⁵⁷⁵, R⁶¹⁰ and R⁶¹⁰ independently of each other, are a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group, or a carbaldehyde (—CHO), a ketone group (—CO—R⁵⁸⁰), a boronic acid group (—B(OH)₂), a cyano group (—C≡N), a carboxylic acid group (—COOH), a carboxylic acid ester group (—COOR⁵⁸¹), a carboxylic acid anhydride group (—CO—O—CO—R⁵⁸²), a hydroxamic acid group (—CO—NH(OH)), a N-substituted hydroxamic acid group (—CO—NR⁵⁸³ (OH)), a O-substituted hydroxamic acid group (—CO—NH(OR⁵⁸⁴)), a carboxamide group (—CO—NH₂), a N-substituted or N,N-disubstituted carboxylic acid amide group, (—CO—NHR⁵⁸⁵; —CO—NR⁵⁸⁶R⁵⁸⁷), an amido group (—HN—CO—R⁵⁸⁸), a sulfonic acid group (—SO₃H), a sulfonamide group (—SO₂—NH₂), a N-substituted or N,N-disubstituted sulfonamide group (—SO₂—NHR⁵⁸⁹; —SO₂—NR⁵⁹OR⁵⁹¹), an amidosulfone group (—NH—SO₂—R⁵⁹²), a sulfone group (—SO₂—R⁵⁹³), a phosphoric acid group (—OP(═O)(OH)₂), a phosphoric acid ester group (—OP(═O)(OR⁵⁹⁴)(OR⁵⁹⁵)), a phosphonic acid group (—P(═O)(OH)₂), an phosphonic acid ester group (—P(═O)(OR⁵⁹⁶)(OR⁵⁹⁷)), a halogen atom, a trifluormethyl group (—CF₃), a thiol group (—SH); a thioether group (—S—R⁵⁹⁸), a hydroxy group (—OH); an alkoxy group (—O—R⁵⁹⁹), a tetrazole group, an amino group (—NH₂), or a N-substituted or N,N-disubstituted amino group (—NHR⁶⁰⁰; —NR⁶⁰¹R⁶⁰²);
which, independently of each other, can be substituted with one or more substituents, which can be the same or different; and,
wherein optionally, the pairs R⁵⁷⁰/R⁵⁷⁵, if present, as well as the pairs R⁵⁸⁶/R⁵⁸⁷, R⁵⁹⁰/R⁵⁹¹, R⁵⁹⁴/R^{595, 596}/R⁵⁹⁷ and R⁶⁰/R⁶⁰², independenly of each other, may form a part of a ring; and
wherein the substituents R⁵⁸⁰, R⁵⁸¹, R⁵⁸², R⁵⁸³, R⁵⁸⁴, R⁵⁸⁵, R⁵⁸⁶, R⁵⁸⁷, R⁵⁸⁸, R⁵⁸⁹, R⁵⁹⁰, R⁵⁹¹, R⁵⁹², R⁵⁹³, R⁵⁹⁴, R⁵⁹⁵, R⁵⁹⁶, R⁵⁹⁷, R⁵⁹⁸, R⁵⁹⁹, R⁶⁰⁰, R⁶⁰¹, and R⁶⁰², independentlyof each other are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅ branched or straight chain alkenoxy, phenyloxy, benzyloxy, C₃, C₄, C₅cycloalkyl, cyano, amido, thiol trifluoromethyl, or hydroxy group; and
or wherein the group PM
has the formula (X) 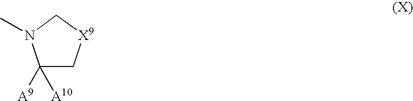
wherein the groups X⁹ is CR⁹⁰⁰R⁹⁰¹, S, SO, SO₂or NR⁹⁰²
wherein R⁹⁰⁰, R⁹⁰¹ and R⁹⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR⁹¹⁰R⁹¹¹.
wherein A⁹ and A¹⁰ are, independently of each other, selected from hydrogen, cyano, —C(═O)NR⁹¹²R⁹¹³, or C₁, C₂, C₃, C₄, C₅or C₆alkyl, which is linear or branched and is optionally substituted with 1, 2, 3, 4, or 5 halogens;
wherein
R⁹¹⁰ and R⁹¹², are, independently of each other, selected from hydrogen, or C₁, C₂, C₃, C₄, C₅or C₆alkyl, which is linear or branched and is optionally substituted with 1, 2, 3, 4, or 5 halogens; and
R⁹¹¹ and R⁹¹³, are, independently of each other, selected from the group consisting of
(1) phenyl, which is optionally substituted with 1, 2, 3, 4, or 5, substituents independently selected from halogen and R⁹²⁰;
(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of
(a) hydroxy,
(b) —COOH,
(c) —COO(C₁, C₂, C₃, C₄, C₅or C₆alkyl), i.e. ester,
(d) phenyl,
(e) naphthyl,
(f) C₃, C₄, C₅or C₆cycloalkyl,
(g) a 5- or 6 membered heterocycle which may be saturated or unsaturated comprising 1, 2, 3, or 4 heteroatoms independently selected from nitrogen , oxygen or sulfur;
(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;
wherein said C₃, C₄, C₅or C₆cycloalkyl, phenyl, naphthyl, are optionally substituted with 1, 2, 3, 4, or 5 substituents independently selected from halogen and R⁹²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R⁹²⁰; and
(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;
wherein R⁹²⁰ is selected from the group consisting of:
(1) hydroxy;
(2) cyano;
(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;
(4) C₁, C₂, C₃, C₄, C₅, C₆, C₇, C₈, C₉or CIO alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from
(a) hydroxy;
(b) —COOH;
(c) —COO(C₁, C₂, C₃, C₄, C₅or C₆alkyl) i.e. ester, which may linear or branched and is optionally substituted with 1, 2, 3, 4, or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(f) —CONR⁹²⁵R⁹²⁵;
(g) —SO₂NR⁹²⁵R⁹²⁵;
(h) —NR⁹²⁵—C(═O)R⁹²
(i) —NR⁹²⁵—C(═O)NR⁹²⁵R⁹²⁵;
)—NR⁹²⁵COOR⁹³⁰
(k) —O—CO—R⁹³⁰
(l) —O—CO—NR⁹²⁵, R⁹²;
(m) —NR⁹²⁵SO₂R⁹³⁰;
(n) —NR⁹²⁵R⁹²⁵;
(o) phenyl which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and
(p) C₃, C₄C₅or C₆cycloalkyl, which is optionally substituted with 1, 2, 3, 4, 5, or 6 halogens;
(5) OC₁, OC₂, OC₃, OC₄, OC₅, OC₆, OC₇, OC₈, OC₉or OC₁₀alkyl, which is linear or branched and is optionally substituted with 0, 1, 2, 3, 4, or 5 halogen atoms and 0, 1, or 2 substitutents selected from
(a) hydroxy;
(b) —COOH;
(c) —COO(C₁, C₂, C₃, C₄, C₅or C₆alkyl) i.e. ester, which may be linear or branched and is optionally substituted with 1, 2, 3, 4 or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, Cs or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(f) —CONR⁹²⁵, R⁹²⁵;
(g) —SO₂NR⁹²⁵, R⁹²;
(h) —NR⁹²—C(═O)R⁹²⁵
(i) —NR⁹²⁵—C(═O)NR⁹²⁵, R⁹²¹
(j) —NR⁹²⁵ COOR⁹³⁰
(k) —NRO -C(930
(l) —O—CO—NR⁹²⁵R⁹²⁵;
(m) —NR⁹²⁵SO₂R⁹³⁰;
(n) _NR⁹²⁵R⁹²⁵;
(o) phenyl, which is optionally substituted with 1, 2, 3, 4, or 5 groups independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and
wherein R¹⁴⁰⁰ is H and R¹⁴⁰¹ is hydrogen atom (—H); or halogen, or cyano or ethynyl;
or wherein the group PM
has the formula (XV) 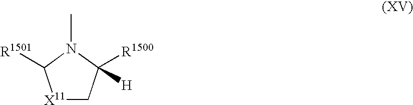
wherein X¹¹ is CH₂, CHF or CF²;
wherein R¹⁵⁰⁰ is cyano;
wherein R¹⁵⁰¹ is selected from the group consisting of alkyl, alkenyl and alkynyl;

9. The compound according to claim 1,
wherein the group PM
has the formula (II) 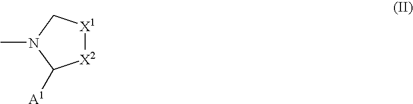
wherein X¹ is CR⁵¹R⁵² or S; and
wherein X² is CR⁵⁴R⁵⁵; and
wherein R⁵¹, R⁵², R⁵⁴, and R⁵⁵, independently of each other, are a hydrogen atom (—H);
wherein A¹ is
a hydrogen atom (—H), or a boronic acid group (—B(OH)₂), a cyano group (—C≡N), or a phosphonic acid ester group (—P(═O)(OR¹¹⁶)(OR¹¹⁷)),
which, independently of each other, can be substituted with one or more substituents, which can be the same or different; and,
wherein optionally, the pairs R¹¹⁶/R¹¹⁷ may form a part of a ring;
wherein the substituents R¹¹⁶ and R¹¹⁷ independently of each other, are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (III) 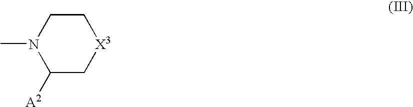
wherein X³ is CR¹³¹, R¹³² or S; and
wherein R¹³¹, R¹³², independently of each other, are a hydrogen atom (—H);
wherein A² is
a hydrogen atom (—H); a boronic acid group (—B(OH)₂), a cyano group (—C≡N), a phosphonic acid ester group (—P(═O)(OR¹⁹⁶)(OR¹⁹⁷));
which, independently of each other, can be substituted with one or more substituents, which can be the same or different; and,
wherein optionally, the pairs R¹⁹⁶/R¹⁹⁷ may form a part of a ring; and
wherein the substituents R¹⁹⁶ and R¹⁹⁷, independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (IV) 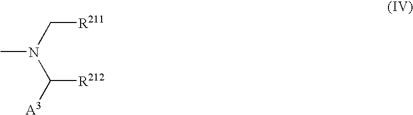
wherein R²¹¹ and R²¹², independently of each other, are
a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group or, an amino group (—NH₂), or a N-substituted or N,N-disubstituted amino group (—NHR²⁴⁰; —NR²⁴R²⁴²); and
which, independently of each other, can be substituted with one or more substituents, which can be the same or different; and,
wherein optionally, the pair R²¹¹I/R²¹², as well the pairs R²²⁶/R²²⁷, R²³⁰/R²³¹, R²³⁴/R²³⁵, R²³⁶/R²³⁷ and R²⁴¹/R²⁴², independenly of each other, may form a part of a ring; and
wherein the substituents R²²⁰, R²², R²²², R²²³, R²²⁴, R²²⁵, R²²⁶, R²²⁷, R²²⁸, R²²⁹, R²³⁰, R²³¹, R²³², R²³³, R²³⁴, R²³⁵, R²³⁶, R²³⁷, R²³⁸, R²³⁹, R²⁴⁰, R²⁴¹, and R²⁴², independently of each other, are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅cycloalkyl, cyano, amido, thiol trifluoromethyl, or hydroxy group; and
wherein A³ is
a hydrogen atom (—H); or a boronic acid group (—B(OH)₂), a cyano group (—C≡N), or a phosphonic acid ester group (—P(═O)(OR²⁷⁶)(OR²⁷⁷))
which, independently of each other, can be substituted with one or more substituents, which can be the same or different; and,
wherein optionally, the pair R²⁷⁶/R²⁷⁷ may form a part of a ring; and
wherein the substituents R²⁷⁶ and R²⁷⁷, independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (V) 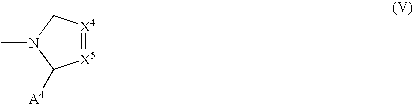
wherein X⁴ is CR²⁹¹ or N; and
wherein X⁵ is CR²⁹² or N; and
wherein R²⁹¹ and R²⁹², independently of each other, are
a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group or an amino group (—NH₂), or a N-substituted or N,N-disubstituted amino group (—NHR³²⁰; —NR³²¹R³²²); and
which, independently of each other, can be substituted with one or more substituents, which can be the same or different; and,
wherein optionally, the the pair R²⁹¹/R²⁹², if present, as well the pairs R³⁰⁶/R³⁰⁷, R³¹⁰/R³¹¹, R³¹⁴/R³¹⁵, R³¹⁶/R³¹⁷ and R³²¹/R³²², independenly of each other, may form a part of a ring; and
wherein the substituents R³⁰⁰, R³⁰¹, R³⁰², R³⁰³, R³⁰⁴, R³⁰⁵, R³⁰⁶, R³⁰⁷, R³⁰⁸, R³⁰⁹, R³¹⁰ R³¹¹, R³¹², R^{313, 4}, R³¹⁵, R³¹⁶, R³¹⁷, R³¹⁸, R³¹⁹, R³²⁰, R³²¹, and R³²², independently of each other are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅cycloalkyl, cyano, amido, thiol trifluoromethyl, or hydroxy group; and
wherein A⁴ is
a hydrogen atom (—H); or a boronic acid group (—B(OH)₂), a cyano group (—C≡N), a phosphonic acid ester group (—P(═O)(OR³⁵⁶)(OR³⁵⁷)),
which, independently of each other, can be substituted with one or more substituents, which can be the same or different; and,
wherein optionally, the pairs R³⁵⁶/R³⁵⁷ may form a part of a ring; and
wherein the substituents R³⁵⁶ and R³⁵⁷, independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (VI) 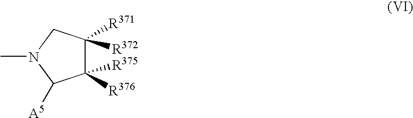
wherein R³⁷¹, R³⁷², R³⁷⁵ and R³⁷⁶, independently of each other, a hydrogen atom (—H); or a C₁, C₂, C₃, C₄, and C₅ branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, and aryl, heteroaryl, aryl-alkyl, heteroaryl-alkyl, aryl-heteroalkyl, heteroaryl-heteroalkyl group or, a carbaldehyde (—CHO), a ketone group (—CO—R³⁸⁰), a boronic acid group (—B(OH)₂), a cyano group (—C≡N), a carboxylic acid group (—COOH), a carboxylic acid ester group (—COOR³⁸¹), a halogen atom, a trifluormethyl group (—CF₃), a thiol group (—SH); a thioether group (—S—R³⁹⁸), a hydroxy group (—OH); an alkoxy group (—O—R³⁹⁹), a tetrazole group, an amino group (—NH₂), or a N-substituted or N,N-disubstituted amino group (—NHR⁴⁰⁰; —NR⁴⁰R⁴⁰²); and
which, independently of each other, can be substituted with one or more substituents, which can be the same or different; and,
wherein optionally, any two of the groups R³⁷¹, R³⁷², R³⁷⁵, and R³⁷⁶, as well as the pairs R³⁸⁶/R³⁸⁷, R³⁹⁰/R³⁹¹, R³⁹⁴/R³⁹⁵, R³⁹⁶/R³⁹⁷ and R⁴⁰¹R⁴⁰², independenly of each other, may form a part of a ring; and
wherein the substituents R³⁸⁰, R³⁸¹, R³⁸², R³⁸³, R³⁸⁴, R³⁸⁵, R³⁸⁶, R³⁸⁷, R³⁸⁸, R³⁸⁹, R³⁹⁰, R³⁹¹, R³⁹², R³⁹³, R³⁹⁴, R³⁹⁵, R³⁹⁶, R³⁹⁷, R³⁹⁸, R³⁹⁹, R⁴⁰⁰, R⁴⁰¹, and R⁴⁰², independently of each other are a hydrogen atom (—H), or a C₁, C₂, C₃, C₄, and C₅branched or straight chain alkyl, aryl, heteroaryl, amino, halo, carbonyl, C₁, C₂, C₃, C₄, C₅, branched or straight chain alkoxy, C₂, C₃, C₄, Cs branched or straight chain alkenoxy, phenyloxy, benzyloxy, C₃, C₄, C₅cycloalkyl, cyano, amido, thiol trifluoromethyl, or hydroxy group; and
alternatively; the two groups R³⁷¹ and R³⁷² can be together an oxo (═O) or hydroxyimino (═N—OH) group; and
alternatively; the two groups R³⁷⁵ and R³⁷⁶ can be together an oxo (═O) or hydroxyimino (═N—OH) group; and
wherein A⁵ is
a hydrogen atom (—H); or a boronic acid group (—B(OH)₂), a cyano group (—C≡N), or a phosphonic acid ester group (—P(═O)(OR⁴³⁶)(OR⁴³⁷));
which, independently of each other, can be substituted with one or more substituents, which can be the same or different; and,
wherein optionally, the pairs R⁴³⁶/R⁴³⁷ may form a part of a ring; and
wherein the substituents R⁴³⁶ and R⁴³⁷, independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (VII) 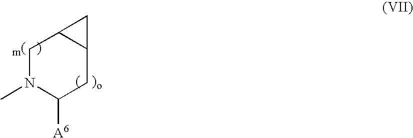
wherein m is equal to 0 and o is equal to 1, or m is equal to 1 and o is equal to 0, or m is equal to 1 and o is equal to 1, or m is equal to 2 and o is equal to 0;
wherein A⁶ is a hydrogen atom (—H); or a boronic acid group (—B(OH)₂), a cyano group (—C≡N), or a phosphonic acid ester group (—P(═O)(OR⁴⁷⁶)(OR⁴⁷⁷)),
which, independently of each other, can be substituted with one or more substituents, which can be the same or different; and,
wherein optionally, the pairs R⁴⁷⁶/R⁴⁷⁷ may form a part of a ring; and
wherein the substituents R⁴⁷⁶ and R⁴⁷⁷, independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (VIII) 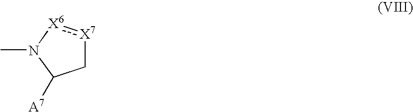
wherein X⁶ is selected from CR⁴⁹⁰R^{, 0} S or NR⁴¹², when the bond between X⁶ and X⁷ is a single bond; and
wherein X⁷ is selected from CR⁴⁹³R⁴⁹⁴, O, S, or NR⁴⁹⁵, when the bond between X⁶ and X⁷ is a single bond;
or alternatively,
wherein X⁶ is selected from CR⁴⁹⁶ or N, when the bond between X⁶ and X⁷ is a double bond; and
wherein X⁷ is selected from CR⁴⁹⁷ or N, when the bond between X⁶ and X⁷ is a double bond; and
wherein R⁴⁹⁰, R⁴⁹¹, R⁴⁹², R⁴⁹³, R⁴⁹⁴, R⁴⁹⁵, R⁴⁹⁶, and R⁴⁹⁷, independently of each other, are a hydrogen atom (—H); or a C₁, C₂, C₃, C₄, and C₅branched or straight chain alkyl, C₂, C₃, C₄, C₅, branched or straight chain alkenyl, C₂, C₃, C₄, C₅, branched or straight chain alkinyl, C₃, C₄, C₅, C₆, and C₇cycloalkyl, aryl, heteroaryl group, or an amino group (—NH₂), or a N-substituted or N,N-disubstituted amino group (—NHR⁵²⁰; —NR⁵²¹R⁵²²); and
which, independently of each other, can be substituted with one or more substituents, which can be the same or different; and,
wherein optionally, any two the groups R⁴⁹⁰, R⁴⁹¹, R⁴⁹², R⁴⁹³, R⁴⁹⁴, R⁴⁹⁵, R⁴⁹⁶, and R⁴⁹⁷, if present, as well as the pairs R⁵⁰⁶/R⁵⁰⁷, R⁵¹⁰/R⁵¹¹, R⁵¹⁴/R⁵¹⁵, R⁵¹⁶/R⁵¹⁷ and R⁵²¹/R⁵²², independenly of each other, may form a part of a ring; and
wherein the substituents R⁵⁰⁰, R⁵⁰¹, R⁵⁰², R⁵⁰³, R⁵⁰⁴, R⁵⁰⁵, R⁵⁰⁶, R⁵⁰⁷, R⁵⁰⁸, R⁵⁰⁹, R⁵¹⁰, R⁵¹¹, R⁵¹², R⁵¹³, R⁵¹⁴, R⁵¹⁵, R⁵¹⁶, R⁵¹⁷, R⁵¹⁸, R⁵¹⁹, R⁵²⁰, R⁵²¹, and R⁵²², independently of each other are a hydrogen atom (—H), or a C₁, C₂, C₃, C₄, and Cs branched or straight chain alkyl, aryl, heteroaryl, amino, halo, carbonyl, C₁, C₂, C₃, C₄, C₅, branched or straight chain alkoxy, C₂, C₃, C₄, C₅ branched or straight chain alkenoxy, phenyloxy, benzyloxy, C₃, C₄, C₅ cycloalkyl, cyano, amido, thiol trifluoromethyl, or hydroxy group; and
wherein A⁷ is
a hydrogen atom (—H); or a carbaldehyde (—CHO), a ketone group (—CO—R⁵⁴⁰), a boronic acid group (—B(OH)₂), a cyano group (—C≡N), a carboxylic acid group (—COOH), a carboxylic acid ester group (—COOR⁵⁴¹), a carboxylic acid anhydride group (—CO—O—CO—R⁵⁴²), a hydroxamic acid group (—CO—NH(OH)), a N-substituted hydroxamic acid group (—CO—NR⁵⁴³ (OH)), a O-substituted hydroxamic acid group (—CO—NH(OR⁵⁴⁴)), a carboxamide group (—CO—NH₂), a N-substituted or N,N-disubstituted carboxylic acid amide group, (—CO—NHR⁵⁴⁵; —CO—NR⁵⁴⁶R⁵⁴⁷), an amido group (—HN—CO—R⁵⁴⁸), a sulfonic acid group (—SO₃H), a sulfonamide group (—SO₂—NH₂), a N-substituted or N,N-disubstituted sulfonamide group (—SO₂—NHR⁵⁴⁹;
—SO₂—NR⁵⁵¹R⁵⁵¹), an amidosulfone group (—NH—SO₂—R⁵⁵²), a sulfone group (—SO₂—R⁵⁵³), a phosphoric acid group (—OP(═O)(OH)₂), a phosphoric acid ester group (—OP(═O)(OR⁵⁵⁴)(oR⁵⁵⁵)), a phosphonic acid group (—P(═O)(OH)₂), an phosphonic acid ester group (—P(═O)(OR⁵⁵⁶)(OR⁵⁵⁷)), a halogen atom, a trifluormethyl group (—CF₃), a thiol group (—SH); a thioether group (—S—R⁵⁵⁸), a hydroxy group (—OH); an alkoxy group (—O—R⁵⁵⁹), a tetrazole group, an amino group (—NH₂), or a N-substituted or N,N-disubstituted amino group (—NHR⁵⁶⁰; —NR⁵⁶, R⁵⁶²); and
which, independently of each other, can be substituted with one or more substituents, which can be the same or different; and,
wherein optionally, the pairs R⁵⁴⁶/R⁵⁴⁷, R⁵⁵⁰/R⁵⁵¹, R⁵⁵⁴/R⁵⁵⁵, R⁵⁵⁶/R⁵⁵⁷ and R⁵⁶¹/R⁵⁶², independenly of each other, may form a part of a ring; and
wherein the substituents R⁵⁴⁰, R⁵⁴, R⁵⁴², R⁵⁴³, R⁵⁴⁴, R⁵⁴⁵, R⁵⁴⁶, R⁵⁴⁷, R⁵⁴⁸, R⁵⁴⁹, R⁵⁵⁰, R⁵⁵¹, R⁵⁵², R⁵⁵³, R⁵⁵⁴, R⁵⁵⁵, R⁵⁵⁶, R⁵⁵⁷, R⁵⁵⁷, R⁵⁶⁰, R⁵⁶¹, and R⁵⁶², independently of each other are a hydrogen atom (—H), or an alkyl, alkenyl, alkinyl, cycloalkyl, cycloalkenyl, cycloalkinyl, heteroalkyl, heteroalkenyl, heteroalkinyl, heterocycloalkyl, heterocycloalkenyl, aryl, heteroaryl, aryl-alkyl, heteroaryl-alkyl, aryl-heteroalkyl, heteroaryl-heteroalkyl group;
or wherein the group PM
has the formula (IX) or (IXa) 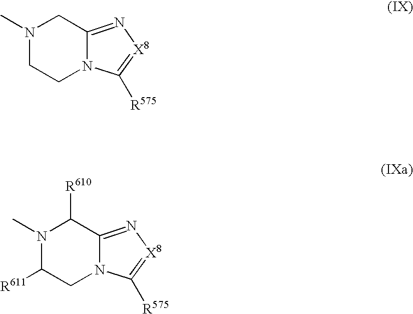
wherein X⁸ is N or CR⁵⁷⁰; and
wherein R⁵⁷⁰, R⁵⁷⁵, R⁶¹⁰ and R⁶¹¹ independently of each other, are
a hydrogen atom (—H), a methyl group (—CH₃), a trifluoromethyl group (—CF₃), an ethyl group (—C₂H₅), a 2,2,2-trifluoroethyl group (—CH₂CF₃), a pentafluoroethyl group (—CF₂CF₃), a phenyl group, (—C₆H₅), a benzyl group (—CH₂—C₆H₅), a benzyloxy group (—OCH₂—C₆H₅), a para-ethyl-phenyl group (—C₆H₄—C₂H₅), a para-fluorophenyl group (—C₆H₄-4-F), a 3,4-difluorophenyl group (—C₆H₃-3,4-F₂), a para-methoxyphenyl group (—C₆H₄-4-OCH₃), a para-trifluoromethoxyphenyl group (—C₆H₄-4-OCF₃), a boronic acid group (—B(OH)₂), a cyano group (—C≡N), a carboxylic acid group (—COOH), or a phosphonic acid ester group (—P(═O)(OR⁵⁹⁶)(OR⁵⁹⁷));
which, independently of each other, can be substituted with one or more substituents, which can be the same or different; and,
wherein optionally, the pairs R⁵⁷⁰/R⁵⁷⁵, if present, as well as the pair R⁵⁹⁶/R⁵⁹⁷, independenly of each other, may form a part of a ring; and
wherein the substituents R⁵⁹⁶ and R⁵⁹⁷, independently of each other are a hydrogen atom (—H), or a C₁, C₂, C₃, C₄, and C₅ branched or straight chain alkyl, aryl, heteroaryl, amino, halo, carbonyl, C₁, C₂, C₃, C₄, C₅, branched or straight chain alkoxy, C₂, C₃, C₄, C₅branched or straight chain alkenoxy, phenyloxy, benzyloxy, C₃, C₄, C₅cycloalkyl, cyano, amido, thiol trifluoromethyl, or hydroxy group; and
or wherein the group PM
has the formula (IX) 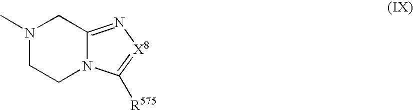
wherein X⁸ is N or CR⁵⁷⁰; and
wherein R⁵⁷⁰ and R⁵⁷⁵, independently of each other, are
(1) hydrogen,
(2) CN,
(3) C₁₋₁₀ alkyl, which is linear or branched and which is unsubstituted or substituted with 1-5 halogens or phenyl, which is unsubstituted or substituted with 1-5 substituents independently selected from halogen, CN, OH, R⁶¹², OR⁶¹², NHSO₂R⁶¹², SO₂R⁶¹², CO₂H, and CO₂C₁₋₆ alkyl, wherein the CO₂C₁₋₆ alkyl is linear or branched,
(4) phenyl which is unsubstituted or substituted with 1-5 substituents independently selected from halogen, CN, OH, R⁶¹², OR⁶¹², NHSO₂R⁶¹², SO₂R⁶¹², CO₂H, and CO₂C₁₋₆ alkyl, wherein the CO₂C₁₋₆ alkyl is linear or branched, and
(5) a 5- or 6-membered heterocycle which may be saturated or unsaturated comprising 1-4 heteroatoms independently selected from N, S, and O, the heterocycle being unsubstituted or substituted with 1-3 substituents independently selected from oxo, OH, halogen, C₁₋₆ alkyl, and OC₁₋₆ alkyl, wherein C₁₋₆ alkyl and C₁₋₆ alkoxy are linear or branched and optionally substituted with 1-5 halogens, and
wherein R⁶¹² is C₁₋₆ alkyl, which is linear or branched and which is unsubstituted or substituted with 1-5 groups independently selected from halogen, CO₂H, and CO₂C₁₋₆ alkyl, wherein the CO₂C₁₋₆ alkyl is linear or branched.
or wherein the group PM
has the formula (IXa) 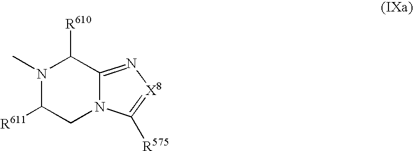
wherein X⁸ is N or CR⁵⁷⁰; and
wherein R⁵⁷⁰ and R⁵⁷⁵ independently of each other, are
(6) hydrogen,
(7) CN,
(8) C₁₋₁₀ alkyl, which is linear or branched and which is unsubstituted or substituted with 1-5 halogens or phenyl, which is unsubstituted or substituted with 1-5 substituents independently selected from halogen, CN, OH, R⁶¹², OR⁶¹², NHSO₂R⁶¹², SO₂R⁶¹², CO₂H, and CO₂C₁₋₆ alkyl, wherein the CO₂C₁₋₆ alkyl is linear or branched,
(9) phenyl which is unsubstituted or substituted with 1-5 substituents independently selected from halogen, CN, OH, R⁶¹², OR⁶¹², NHSO₂R⁶¹², SO₂R⁶¹², CO₂H, and CO₂C₁₋₆ alkyl, wherein the CO₂C₁₋₆ alkyl is linear or branched, and
(10)a 5- or 6-membered heterocycle which may be saturated or unsaturated comprising 1-4 heteroatoms independently selected from N, S, and O, the heterocycle being unsubstituted or substituted with 1-3 substituents independently selected from oxo, OH, halogen, C₁₋₆ alkyl, and OC₁₋₆ alkyl, wherein C₁₋₆ alkyl and C₁₋₆ alkoxy are linear or branched and optionally substituted with 1-5 halogens, and
wherein R⁶¹² is C₁₋₆ alkyl, which is linear or branched and which is unsubstituted or substituted with 1-5 groups independently selected from halogen, CO₂H, and CO₂C₁₋₆ alkyl, wherein the CO₂C₁₋₆ alkyl is linear or branched, and
wherein R⁶¹ and R⁶¹¹ are each independently selected from the group consisting of
(1) hydrogen,
(2) C₁₋₁₀ alkyl, which is linear or branched and which is unsubstituted or substituted with one or more substituents selected from:
(a) halogen,
(b) hydroxy,
(c) phenyl, wherein the phenyl is unsubstituted or substituted with 1-5 substituents independently selected from halogen, OH, C₁₋₆ alkyl, and C₁₋₆ alkoxy, wherein the C₁₋₆ alkyl, and C₁₋₆ alkoxy are linear or branched and optionally substituted with 1-5 halogens,
(d) naphthyl, wherein the naphthyl is optionally substituted with 1-5 substituents independently selected from halogen, CN, OH, C₁₋₆ alkyl, and C₁₋₆ alkoxy, wherein the C₁₋₆alkyl, and C₁₋₆alkoxy are linear or branched and optionally substituted with 1-5 halogens,
(e) CO₂H,
(f) CO₂C₁₋₆ alkyl,
(g) CONR⁶¹³R⁶¹¹, wherein R⁶¹³ and R⁶¹⁴ are independently selected from the group consisting of hydrogen, tetrazolyl, phenyl, C₃₋₆ cycloalkyl and C₁₋₆ alkyl, wherein the C₁₋₆alkyl is linear or branched and is optionally substituted with 1-6 substituents independently selected from 0-5 halogen and 0-1 phenyl, wherein the phenyl or the C₃₋₆ cycloalkyl beeing R⁶¹³ and R⁶¹⁴ or the optional phenyl substituent on the C₁₋₆ alkyl are optionally substituted with 1-5 substituents independently selected from halogen, OH, C₁₋₆alkyl, and OC₁₋₆ alkyl, said C₁₋₆alkyl and OC₁₋₆ alkyl being linear or branched and optionally substituted with 1-5 halogens, or wherein R⁶¹³ and R⁶¹⁴ are optionally joined to form a ring selected from pyrrolidine, piperidine or morpholine,
(3) CN,
(4) phenyl, wherein the phenyl is unsubstituted or substituted with 1-5 substituents independently selected from C₁₋₆ alkyl, and C₁₋₆ alkoxy, hydroxy and halogen, wherein the C₁₋₆ alkyl, and C₁₋₆ alkoxy are linear or branched and optionally substituted with 1-5 halogens,
(5) naphthyl, wherein the naphthyl is unsubstituted or substituted with 1-5 substituents independently selected from halogen, OH, C₁₋₆ alkyl, and C₁₋₆ alkoxy, wherein the C₁₋₆ alkyl, and C₁₋₆ alkoxy are linear or branched and optionally substituted with 1-5 halogens,
(6) CO₂H,
(7) CO₂C₁₋₆ alkyl,
(8) CONR⁶¹³, R⁶¹⁴, and
(9) C₃₋₆ cycloalkyl, which is optionally substituted with 1-5 substituents independently selected from halogen, OH, C₁₋₆ alkyl, and C₁₋₆alkoxy, wherein the C₁₋₆alkyl, and C₁₋₆ alkoxy are linear or branched and optionally substituted with 1-5 halogen, with the proviso that one of R⁶¹⁰ and R⁶¹¹ is other than hydrogen.
or wherein the group PM
has the formula (X) 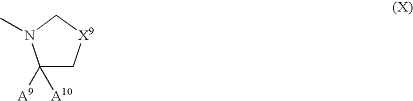
wherein the groups X⁹ is CR⁹⁰⁰R⁹⁰¹, S, SO, SO₂or NR⁹⁰²
wherein R⁹⁰⁰, R⁹⁰¹ and R⁹⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR⁹¹⁰R⁹¹¹.
wherein A⁹ and A¹⁰ are, independently of each other, selected from hydrogen, cyano, —C(═O)NR⁹¹²R⁹¹³, or C₁, C₂, C₃, C₄, C₅or C₆alkyl, which is linear or branched and is optionally substituted with 1, 2, 3, 4, or 5 halogens;
wherein
R⁹¹⁰ and R⁹¹², are, independently of each other, selected from hydrogen, or C₁, C₂, C₃, C₄, C₅or C₆alkyl, which is linear or branched and is optionally substituted with 1, 2, 3, 4, or 5 halogens; and
R⁹¹¹ and R⁹¹³, are, independently of each other, selected from the group consisting of
(1) phenyl, which is optionally substituted with 1, 2, 3, 4, or 5, substituents independently selected from halogen and R⁹²⁰;
(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of
(a) hydroxy,
(b) —COOH,
(c) —COO(C₁, C₂, C₃, C₄, C₅or C₆alkyl), i.e. ester,
(d) phenyl,
(e) naphthyl,
(f) C₃, C₄, C₅or C₆cycloalkyl,
(g) a 5- or 6 membered heterocycle which may be saturated or unsaturated comprising 1, 2, 3, or 4 heteroatoms independently selected from nitrogen , oxygen or sulfur;
(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;
wherein said C₃, C₄, C₅or C₆cycloalkyl, phenyl, naphthyl, are optionally substituted with 1, 2, 3, 4, or 5 substituents independently selected from halogen and R⁹²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R⁹²⁰; and
(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;
wherein R⁹²⁰ is selected from the group consisting of:
(1) hydroxy;
(2) cyano;
(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;
(4) C₁, C₂, C₃, C₄, C₅, C₆, C₇, C₈, C₉or C₁₀alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from
(a) hydroxy;
(b) —COOH;
(c) —COO(C₁, C₂, C₃, C₄, C₅or C₆alkyl) i.e. ester, which may linear or branched and is optionally substituted with 1, 2, 3, 4, or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(f) —CONR⁹²⁵R⁹²⁵;
(g) —SO₂NR⁹²⁵R⁹²⁵;
(h) —NR⁹²⁵—C(═O)R⁹²⁵
(i) —NR⁹²⁵C(═O)NR⁹²⁵R⁹²⁵
(j)—NR⁹²⁵COOR⁹³⁰
(k) —O—CO—R⁹³⁰
(l) —O—CO—NR⁹²⁵, R⁹²⁵;
(m) —NR⁹²⁵SO⁹³⁰;
(n) NR⁹²⁵R⁹²⁵;
(o) phenyl which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and
(p) C₃, C₄C₅or C₆cycloalkyl, which is optionally substituted with 1, 2, 3, 4, 5, or 6 halogens;
(5) OC₁, OC₂, OC₃, OC₄, OC₅, OC₆, OC₇, OC₈, OC₉or OC₁₀ alkyl, which is linear or branched and is optionally substituted with 0, 1, 2, 3, 4, or 5 halogen atoms and 0, 1, or 2 substitutents selected from
(a) hydroxy;
(b) —COOH;
(c) —COO(C₁, C₂, C₃, C₄, C₅or C₆alkyl) i.e. ester, which may be linear or branched and is optionally substituted with 1, 2, 3, 4 or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(f) —CONR⁹²⁵, R⁹²⁵;
(g) —SO₂NR⁹²⁵R⁹²⁵;
(h) —NR⁹²⁵—C(═O)R⁹²⁵
(i) —NR⁹²⁵C(═O)NR⁹²⁵, R⁹²⁵;
(j)—NR⁹²⁵COOR⁹³⁰
(k) —O—CO—R⁹³⁰
(l) —O—CO—NR⁹²⁵R⁹²⁵;
(m) —NR⁹²⁵SO₂R⁹³⁰;
(n) —NR⁹²⁵R⁹²⁵;
(o) phenyl, which is optionally substituted with 1, 2, 3, 4, or 5 groups independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and
(p) C₃, C₄C₅or C₆cycloalkyl, which is optionally substituted with 1, 2, 3, 4, 5, or 6 halogens;
(6) —COOH;
(7) —COO(C₁, C₂, C₃, C₄, C₅or C₆alkyl) i.e. ester, which may be linear or branched and is optionally substituted with 1, 2, 3, 4, 5 halogens;
(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.
(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, -OC₅or —OC₆alkyl being linear or branched and optionally substituted with 1, 2, 3, 4, or 5 halogens;
(10) —CONR⁹²⁵R⁹²⁵;
(11) —SO₂NR⁹²⁵R⁹²⁵;
(12) —NR⁹²⁵—C(═O)R⁹²⁵
(13)—NR⁹²⁵—C(═O)NR⁹²⁵R⁹²⁵;
(14) —NR⁹²⁵COOR⁹³⁰
(15)—O—CO—R⁹³⁰
(16)—O—CO—NR⁹²⁵, R⁹²⁵
(17) NR⁹²⁵SO₂R⁹³⁰;
(18)—NR⁹²⁵, R⁹²⁵;
(19) phenyl , which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;
wherein R⁹³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, —OC₃, —OC₄, or —OC₅alkyl, said C₁, C₂, C₃, C₄, or C₅alkyl, —OC₁, —OC₂, —OC₃, —OC₄, or —OC₅alkyl being linear or branched and optionally substituted with 1, 2, 3, 4, or 5 halogens.
wherein R⁹²⁵ is selected from R⁹³⁰ and hydrogen.
wherein the group PM
has the formula (XI) 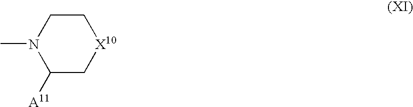
wherein the groups X¹⁰ is CR¹⁰⁰⁰R¹⁰⁰¹, S, SO, SO₂or NR¹⁰⁰²
wherein R¹⁰⁰⁰, R¹⁰⁰¹ and R¹⁰⁰², are, independently of each other, selected from hydrogen, fluorine, C₁, C₂, C₃, C₄, C₅or C₆alkyl, which is linear or branched and is optionally substituted with 1, 2, 3, 4, or 5 halogens, or —C(═O)NR¹⁰¹⁰OR¹⁰¹¹.
and A¹¹ is selected from
hydrogen, cyano, —C(═O)NR¹⁰¹²R¹⁰¹³, or C₁, C₂, C₃, C₄, C₅or C₆alkyl, which is linear or branched and is optionally substituted with 1, 2, 3, 4, or 5 halogens;
wherein
R¹⁰¹⁰ and R¹⁰¹², are, independently of each other, selected from hydrogen, or C₁, C₂, C₃, C₄, C₅or C₆alkyl, which is linear or branched and is optionally substituted with 1, 2, 3, 4, or 5 halogens; and
R¹⁰¹¹ and R¹⁰¹³, are, independently of each other, selected from the group consisting of
(1) phenyl, which is optionally substituted with 1, 2, 3, 4, or 5, substituents independently selected from halogen and R¹⁰²¹;
(2) C₁, C₂, C₃, C₄, C₅or C₆alkyl, which is linear or branched and is optionally substituted with 1, 2, 3, 4, 5, 6 or 7 substitutents independently selected from (a) 0, 1, 2, 3, 4, or 5 halogens, and (b) 0, 1, 2 substituents selected from the group consisting of
(a) hydroxy,
(b) —COOH,
(c) —COO(C₁, C₂, C₃, C₄, C₅or C₆alkyl), i.e. ester,
(d) phenyl,
(e) naphthyl,
(f) C₃, C₄, C₅or C₆cycloalkyl,
(g) a 5- or 6 membered htereocycle which may be saturated or unsaturated comprising 1, 2, 3, or 4 heteroatoms independently selected from nitrogen , oxygen or sulfur;
(h) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a benzene ring fused to a 5- or 6-membered heterocycle having 1, 2, or 3 hetero atoms;
wherein said C₃, C₄, C₅or C₆cycloalkyl, phenyl, naphthyl, are optionally substituted with 1, 2, 3, 4, or 5 substituents independently selected from halogen and R¹⁰²⁰, and said 5 or 6 membered heterocycle and said 8, 9 or 10-membered bicyclic ring system are each optionally substituted with 1, 2, 3, 4, or 5 substituents independently selected from from oxo, hodroxy, halogen, and R¹⁰²⁰; and
(3) C₃, C₄C₅or C₆cycloalkyl, which is optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, said —COO(C₁, C₂, C₃, C₄, C₅or C₆alkyl), i.e. ester, C₁, C₂, C₃, C₄, C₅or C₆ alkyl, and —OC₁, —OC₂, —OC₃, —OC₄, —OC₅or —OC₆alkyl being linear or branched and optionally substituted with 1, 2, 3, 4, or 5 halogens;
wherein R¹⁰²⁰ is selected from the group consisting of:
(1) hydroxy;
(2) cyano;
(3) C₃, C₄C₅or C₆cycloalkyl optionally substituted with 1, 2, or 3 groups independently selected from halogen, hydroxy, —COOH, —COO(C₁, C₂, C₃, C₄, C₅or C₆ alkyl), i.e. ester, C₁, C₂, C₃, C₄, C₅or C₆alkyl, and —OC₁, —OC₂, —OC₃, —OC₄, —OC₅or —OC₆alkyl, wherein said —COO(C₁, C₂, C₃, C₄, C₅or C₆alkyl) i.e. ester, C₁, C₂, C₃, C₄, C₅or C₆alkyl, and —OC₁, —OC₂, —OC₃, —OC₄, —OC₅or —OC₆alkyl are linear or branched and are optionally substituted with 1, 2, 3, 4, 5 or 6 substituents selected from 1, 2, 3, 4, or 5 halogens, and 0 or 1 substituents selected from —COO(C₁, C₂, C₃, C₄, C₅or C₆ alkyl) i.e. ester, —COOH, and —OC₁, —OC₂, —OC₃, —OC₄, —OC₅or —OC₆alkyl substituents being linear or branched and optionally substituted with 1, 2, 3, 4, or 5 halogens;
(4) C₁, C₂, C₃, C₄, C₅, C₆, C₇, C₈, C₉or Clo alkyl, which is linear or branched and is optionally substituted with 1, 2, 3, 4, 5, 6, or 7 substituents independently selected from 0, 1, 2, 3, 4, or 5 halogen atoms and 0, 1, or 2 groups selected from
(a) hydroxy;
(b) —COOH;
(c) —COO(C₁, C₂, C₃, C₄, C₅or C₆alkyl) i.e. ester, which may linear or branched and is optionally substituted with 1, 2, 3, 4, or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle havoing 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(f) —CONR¹⁰²⁵R¹⁰²⁵;
(g) —SO₂NR¹⁰²⁵R¹⁰²⁵
(h) —NR¹⁰²⁵—C(═O)R¹⁰²⁵
(i) —NR¹⁰²⁵—C(═O)NR¹⁰²⁵R¹⁰²⁵
(j)—NR¹⁰²⁵COOR¹⁰³⁰
(k) —O—CO—R¹⁰³⁰
(l) —O—CO—NR¹⁰²⁵R¹⁰²⁵;
(m) —NR¹⁰²⁵, R¹⁰³⁰;
(n) —NR¹⁰²⁵R¹⁰²⁵
(o) phenyl which is optionally substituted with 1, 2, 3, 4, or 5 group independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and
(p) C₃, C₄C₅or C₆cycloalkyl, which is optionally substituted with 1, 2, 3, 4, 5, or 6 halogens;
(5) OC₁, OC₂, OC₃, OC₄, OC₅, OC₆, OC₇, OC₈, OC₉or OC₁₋₁₀ alkyl, which is linear or branched and is optionally substituted with 0, 1, 2, 3, 4, or 5 halogen atoms and 0, 1, or 2 substitutents selected from
(a) hydroxy;
(b) —COOH;
(c) —COO(C₁, C₂, C₃, C₄, C₅or C₆alkyl) i.e. ester, which may be linear or branched and is optionally substituted with 1, 2, 3, 4 or 5 halogens;
(d) a 5- or 6-membered heterocycle which may be saturated or unsaturated comprising 1, 2, 3, or 4 hetero atoms independently selected from nitrogen, oxygen and sulfur, optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.;
(e) an 8, 9 or 10 membered bicyclic ring system which may be saturated or unsaturated comprising (i) two fused heterocyclic rings, each heterocyclic ring having 1, 2, 3, or 4 heteroatoms independently selected from nitrogen, oxygen or sulfur, or (ii) a 5- or 6-membered heterocycle having 1, 2, or 3 heteroatoms independently selected from nitrogen , oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅ or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(f) —CONR¹⁰²⁵R¹⁰²⁵;
(g) —SO₂NR¹⁰²⁵R¹⁰²⁵;
(h) —NR¹⁰²⁵—C(═O)R¹⁰²⁵
(i) —NR¹⁰²⁵—C(═O)NR¹⁰²⁵R¹⁰²⁵
(j)—NR¹⁰²⁵COOR¹⁰³⁰
(k) —O—CO—R¹⁰³⁰
(l) —O—CO—NR¹⁰²⁵R¹⁰²⁵;
(m) —NR⁰ SO₂R¹⁰³⁰;
(n) —NR¹⁰²⁵R¹⁰²⁵;
(o) phenyl, which is optionally substituted with 1, 2, 3, 4, or 5 groups independently selected from halogen, hydroxy, C₁, C₂, C₃, C₄, C₅ or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, 5, or 6 substitutents independently selected from 0 or 1 C₃, C₄C₅or C₆cycloalkyl and 0, 1, 2, 3, 4, or 5 halogens, and
(p) C₃, C₄C₅or C₆cycloalkyl, which is optionally substituted with 1, 2, 3, 4, 5, or 6 halogens;
(6) —COOH;
(7) —COO(C₁, C₂, C₃, C₄, C₅or C₆alkyl) i.e. ester, which may be linear or branched and is optionally substituted with 1, 2, 3, 4, 5 halogens;
(8) a 5- or 6-membered heterocycle which may be saturated or unsaturated comprising 1, 2, 3, or 4 hetero atoms independently selected from nitrogen, oxygen and sulfur, said heterocycle being optionally substituted with 1, 2, or 3 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens.
(9) an 8, 9 or 10 membered bicyclic ring system which may be saturated or unsaturated comprising (a) two fused heterocyclic rings, each heterocyclic ring having 1, 2, 3, or 4 heteroatoms independently selected from nitrogen, oxygen or sulfur, or (b) a 5- or 6-membered heterocycle having 1, 2, or 3 heteroatoms independently selected from nitrogen, oxygen and sulfur, fused to a benzene ring, wherein said bicyclic ring system is optionally substituted with 1, 2, 3, 4, or 5 substituents independently selected from oxo, hydroxy, halogen, C₁, C₂, C₃, C₄, C₅or C₆alkyl, and —OC₁, —OC₂, —OC₃, —OC₄, —OC₅or —OC₆alkyl, said C₁, C₂, C₃, C₄, C₅or C₆alkyl, and —OC₁, —OC₂, —OC₃, —OC₄, —OC₅or —OC₆alkyl being linear or branched and optionally substituted with 1, 2, 3, 4, or 5 halogens;
(10) —CONR¹⁰²⁵R¹⁰²⁵;
(11) —SO₂NR¹⁰²⁵R¹⁰²⁵;
(12) —NR¹⁰²⁵—C(═O)R¹⁰²⁵
(13)—NR¹⁰²⁵—C(═O)NR¹⁰²⁵, R¹⁰²⁵
(14)—NR⁹²⁵COOR¹⁰³⁰
(15)—O—CO—R¹⁰³⁰
(16) —O—CO—NR¹⁰²⁵R¹⁰²⁵;
(17) NR¹⁰²⁵ SO₂R¹⁰³⁰;
(18)—NR¹⁰²⁵R¹⁰²⁵;
(19) phenyl , which is optionally substituted with 1, 2, 3, 4, or 5 group independently selected from halogen, hydroxy, C₁, C₂, C₃, C₄, Cs or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester, said C₁, C₂, C₃, C₄, C₅or C₆alkyl, —OC₁, —OC₂, —OC₃, —OC₄, —OC₅or —OC₆alkyl, —COOH, —COO(C₁, C₂, C₃, C₄, C₅or C₆alkyl) i.e. ester being linear or branched and optionally substituted with 1, 2, 3, 4, or 5 halogens;
wherein R¹⁰³⁰ is selected from the group consisting of phenyl, C₃, C₄C₅or C₆ cycloalkyl, and C₃, C₄C₅or C₆cycloalkyl, wherein C₁, C₂, C₃, C₄, C₅or C₆alkyl is linear or branched anbd is optionally substituted with 1, 2, 3, 4, 5, 6, substitutents independently selected from 0, 1, 2, 3, 4, or 5 halogens, 0 or 1 phenyl, wherein said optional phenyl substituent and said R⁹³⁰, when R⁹³⁰ is phenyl or C₃, C₄C₅or C₆ cycloalkyl, are optionally substituted with 1, 2, 3, 4, or 5 substituents, independently selected from halogen, OH, C₁, C₂, C₃, C₄, or C₅alkyl, —OC₁, —OC₂, —OC₃, —OC₄, or —OC₅alkyl, said C₁, C₂, C₃, C₄, or C₅ alkyl, —OC₁, —OC₂, —OC₃, —OC₄, or —OC₅alkyl being linear or branched and optionally substituted with 1, 2, 3, 4, or 5 halogens,
wherein R¹⁰²⁵ is selected from R¹⁰³⁰ and hydrogen.
or wherein the group PM
has the formula (XII) 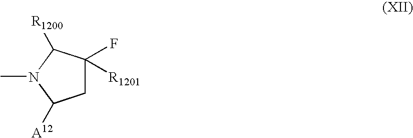
wherein the groups R¹²⁰¹ is hydrogen or fluoro.
wherein R¹²⁰⁰ und A¹² is selected from hydrogen and cyano, and the other is hydrogen.
or wherein the group PM
has the formula XIII: 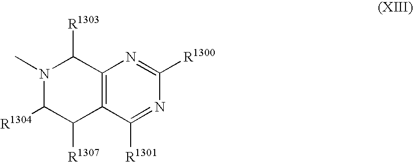
wherein:
R¹³⁰⁰ is selected from the group consisting of:
(1) hydrogen,
(2) CN,
(3) C₁₋₁₀alkyl, which is linear or branched which is unsubstituted or substituted with:
a) halogen, or
b) phenyl, which is unsubstituted or substituted with 1-5 substituents independently selected from halogen, CN, OH, R¹³⁰², OR¹³⁰² NHSO₂R¹³⁰², N(C₁₋₆alkyl)SO₂R¹³⁰², SO₂R¹³⁰², SO₂NR¹³⁰⁵R¹³⁰⁶, NR¹³⁰⁵R¹³⁰⁶CONR¹³⁰⁵R¹³⁰⁶CO₂H and CO₂C₁₋₆alkyl, wherein the C₁₋₆alkyl is linear or branched,
(4) phenyl which is unsubstituted or substituted with 1-5 substituents independently selected from halogen, CN, OH, R¹³⁰², OR¹³⁰², NHSO₂R¹³⁰², N(C₁₋₆alkyl)SO₂R¹³⁰², SO₂R¹³⁰², SO₂NR¹³⁰⁵R¹³⁰⁶, NR¹³⁰⁵R¹³⁰⁶, COR¹³⁰⁵R¹³⁰⁶, CO₂H, and CO₂C₁₋₆alkyl, wherein the C₁₋₆alkyl is linear or branched,
(5) a 5- or 6-membered heterocyclic which may be saturated or unsaturated comprising 1-4 heteroatoms independently selected from N, S and O, the heterocycle being unsubstituted or substituted with 1-3 substituents independently selected from oxo, halogen, NO₂, CN, OH, R¹³⁰², OR¹³⁰², NHSO₂R¹³⁰², N(C₁₋₆alkyl)SO₂R¹³⁰², SO₂R¹³⁰², SO₂NR¹³⁰⁵R¹³⁰⁶, NR¹³⁰⁵R¹³⁰⁶, CONR¹³⁰⁵, R¹³⁰⁶ CO₂H, and CO₂C₁₋₆alkyl, wherein the C₁₋₆alkyl is linear or branched,
(6) C₃₋₆cycloalkyl, which is optionally substituted with 1-5 substituents independently selected from halogen, OH, C₁₋₆alkyl, and OC₁₋₆alkyl, wherein the C₁₋₆alkyl and OC₁₋₆ alkyl are linear or branched and optionally substituted with 1-5 halogens,
(7) OH,
(8) OR¹³⁰², and
(9) NR¹³⁰⁵R¹³⁰⁶;
R³⁰¹ is hydrogen;
R¹³⁰²is C₁₋₆alkyl, which is linear or branched and which is unsubstituted or substituted with 1-groups independently selected from halogen, CO₂H, and CO₂C₁₋₆alkyl, wherein the C₁₋₆alkyl is linear or branched;
R¹³⁰³ is hydrogen;
R¹³⁰⁵ and R¹³⁰⁶ are independently selected from the group consisting of:
(1) hydrogen,
(2) phenyl, which is unsubstituted or substituted with substituents independently selected from halogen, OH, C₁₋₆alkyl, and OC₁₋₆alkyl, wherein the C₁₋₆alkyl is linear or branched and optionally substituted with 1-5 halogens
(3) C₃₋₆cycloalkyl, which is unsubstituted or substituted with 1-5 substituents independently selected from C₁₋₆alkyl, and OC₁₋₆alkyl, wherein the C₁₋₆alkyl is linear or branched and optionally substituted with 1-5 halogens
(4) C₁₋₆alkyl, which is linear or branched and which is unsubstituted or substituted with:
a) halogen, or
b) phenyl, which is unsubstituted or substituted with 1-5 substituents independently selected from halogen, OH, C₁₋₆alkyl, and OC₁₋₆alkyl, wherein the C₁₋₆alkyl is linear or branched and optionally substituted with 1-5 halogens,
or wherein R¹³⁰⁵ and R¹³⁰⁶ together with the nitrogen atom to which they are attached form a heterocyclic ring selected from azetidine, pyrrolidine, piperidine, piperazine, and morpholine wherein said heterocyclic ring is unsubstituted or substituted with one to five substituents independently selected from halogen, hydroxy, C₁₋₆alkyl, and C₁₋₆alkoxy, wherein alkyl and alkoxy are unsubstituted with one to five halogens;
R¹³⁰⁴ and R¹³⁰⁷ are hydrogen;
or wherein the group PM
has the formula XIV: 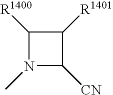
wherein R¹⁴⁰⁰ is H and R¹⁴⁰¹ is hydrogen atom (—H); or fluoro, or cyano.

10. A composition comprising a compound according to claim 1 in combination with acarbose.

11. A composition comprising a compound according to claim 1 in combination with metformin.

12. A composition comprising a compound according to claim 1 in combination with acarbose and metformin.

13. A composition comprising a compound according to claim 1 in combination with
(a) other DP IV inhibitors
(b) insulin sensitizers selected from the group consisting of
(i) PPAR agonists,
(ii) biguanides, and
(iii) protein tyrosin phosphatase-1B (PTP-1B) inhibitors;
(c) insulin and insulin mimetics;
(d) sulfonylureas and other insulin secretagogues;
(e) α-glucosidase inhibitors;
(f) glucagon receptor agonists;
(g) GLP-1; GLP-1 mimetics, e.g. NN-2211 (liraglutide from Novo Nordisk), and GLP-1 receptor agonists;
(h) GLP-2; GLP-2 mimetics, e.g. ALX-0600 (teduglutide from NPS Allelix Corp.) and GLP-2 receptor agonists;
(i) exendin-4 and exendin-4 mimetics, e.g. exenatide (AC-2993, synthetic exendin-4 from Amylin/Eli Lilly);
(j) GIP, GIP mimetics, and GIP receptor agonists;
(k) PACAP, PACAP mimetics, and PACAP receptor 3 agonists;
(l) choletserol lowering agents selected from the group consisting of
(i) HMG-CoA reductase inhibitors,
(ii) sequestrants,
(iii) nicotinyl alkohol, nicotinic acid and salts thereof,
(iv) PPARα agonists,
(v) PPARα/γ dual agonists,
(vi) inhibitors of cholesterol absorption,
(vii) acyl CoA:cholesterol acyltransferase inhibitors, and
(viii) antioxidants;
(m) PPARδ agonists;
(n) antiobesity compounds;
(o) an ileal bile acid transporter inhibitor; and
(p) anti-inflammatory agents.

14. A composition comprising a compound according to claim 1 in combination with a gene therapeutic expression system for GLP-1 comprising a viral vector comprising
(a) a polynucleotide sequence encoding GLP-1 (gluacogen like peptide -1); and
(b) a polynucleotide sequence encoding a signal sequence upstream of (a); and
(c) a polyadenylation signal downstream of (a); and
(d) a polynucleotide sequence encoding a proteolytic cleavage site located between the polynucleotide sequence encoding GLP-1 and the polynucleotide sequence encoding the signal sequence; and
(e) wherein the expression of GLP-1 underlies a constitutive promoter or is controlled by a regulatable promotor;
(f) wherein, optionally, the viral vector comprises a polynucleotide sequence encoding GIP (glucose dependent insulinotropic peptide);
(g) wherein, optionally, the viral vector is encompassed by a mammalian cell.

15. A composition comprising a compound according to claim 1 in combination with a gene therapeutic expression system for GIP comprising a viral vector comprising
(a) a polynucleotide sequence encoding GIP (glucose dependent insulinotropic peptide); and
(b) a polynucleotide sequence encoding a signal sequence upstream of (a); and
(c) a polyadenylation signal downstream of (a); and
(d) a polynucleotide sequence encoding a proteolytic cleavage site located between the polynucleotide sequence encoding GIP and the polynucleotide sequence encoding the signal sequence; and
(e) wherein the expression of GIP underlies a constitutive promoter or is controlled by a regulatable promotor;
(f) wherein, optionally, the viral vector comprises a polynucleotide sequence encoding GLP-1 (glucagon like peptide 1);
(g) wherein, optionally, the viral vector is encompassed by a mammalian cell.

16. A composition comprising a compound according to claim 1 in combination with a gene therapeutic expression system for GLP-1 and/or GIP
wherein
the signal sequence upstream of the gene of interest (GLP-1; GIP) is the murine immunoglobulin K signal sequence or the glia monster exendin signal sequence; and/or
the polyadenylation signal downstream of the gene of interest (GLP-1; GIP) is derived from simian viraus 40 (SV 40); and /or
the proteolytic cleavage site is cleaved by furin preotease; and/or
the gene delivery vector for expression the gene of interest is an adenoviral, retroviral, leniviral, adeno associated viral vector; and /or the constitutive promoter is a cytomegalovirus (CMV) promotor, or a Rous sarcoma long-terminal repeat (LTR) sequence, and the SV 40 early gene gene promoter; and the inducible promoter is the Tet-On™/Tet-Off™ system available from Clontech; and /or
the mammalian cell is a primate or rodent cell, preferably a human cell, more preferably a human hepatocyte.

17. A composition according to claim 11, which additionally comprises an inhibitor of glutaminyl cyclase.

18. A Pharmaceutical composition comprising a compound according to claim, 1 and a pharmaceutical acceptable diluent and/or carrier.

19-22. (Cancelled)

23. A method for treatment of disorders related to the inhibition of dipeptidyl peptidase IV and dipeptidyl peptidase IV-like enzyme activity in a mammal comprising the step of administering to a mammal a therapeutically effective amount of a composition according to claim 11.

24. (Cancelled)

25. The method according to claim 23 for the treatment of disorders selected from the group consisting of non-insulin dependent diabetes mellitus (type 2), impaired glucose tolerance, impaired fasting glucoase, impaired glucose metabolism, prediabetes, glucosuria, and disturbances of signal action at the cells of the islets of Langerhans and insulin sensitivity in the peripheral tissue in the postprandial phase of mammals, insulin resistance, lipid disorders, hyperlipidemia, metabolic acidosis, diabetic neuropathy and nephropathy and of sequelae caused by diabetes mellitus in mammals; obesity, metabolism-related hypertension and cardiovascular sequelae caused by hypertension in mammals; atherosclerosis and its sequelae, inflammatory bowel disease, Crohn's disease, ulcerative colitis, inflammatory conditions, pancreatitis, tumor metastasis, benign prostatic hypertrophy, gingivitis, osteoporosis, prohylaxis of treatment of skin diseases, diseases of the mucosae, autoimmune diseases, treatment of psychosomatic, neuropsychiatric depressive illness, anxiety, depression, sleep disorders, chronic fatigue, schizophrenia, epilepsy, nutritional disorders, spasm, and chronic pain.

26. The method according to claim 25 for the treatment of disorders selected from the group consisting of non-insulin dependent diabetes mellitus (type 2), prediabetes, impaired glucose tolerance, impaired fasting glucoase and impaired glucose metabolism.
